(19) United States
(12) Patent Application Publication
(10) Pub. No.: US 2026/0255775 A1
(43) Pub. Date: Aug. 27, 2026

(54) ORGANIC ELECTROLUMINESCENT ELEMENT AND ELECTRONIC DEVICE

(71) Applicant: IDEMITSU KOSAN CO.,LTD., Tokyo (JP)

(72) Inventors: Hiroaki TOYOSHIMA, Tokyo (JP); Tetsuya MASUDA, Tokyo (JP); Yusuke TAKAHASHI, Tokyo (JP); Hiroaki ITOI, Tokyo (JP)

(73) Assignee: IDEMITSU KOSAN CO.,LTD., Tokyo (JP)

(21) Appl. No.: 18/851,731

(22) PCT Filed: Mar. 28, 2023
(86) PCT No.: PCT/JP2023/012669

§ 371 Date: Sep. 27, 2024

(30) Foreign Application Priority Data
Mar. 29, 2022 (JP) 2022-054252
Dec. 27, 2022 (JP) 2022-210031

(51) Int. Cl. H10K 50/11; C09K 11/06; H10K 50/15; H10K 85/60; H10K 101/00; H10K 101/10

(52) U.S. Cl. CPC H10K 50/11; C09K 11/06; H10K 50/156; H10K 85/622; H10K 85/623; H10K 85/626; H10K 85/633; H10K 85/636; H10K 85/654; H10K 85/6572; H10K 85/6574; H10K 85/6576; H10K 85/658; C09K 2211/1088; H10K 2101/10; H10K 2101/90

(57) ABSTRACT

An organic electroluminescence device includes an emitting zone including a first emitting layer and a second emitting layer, in which a triplet energy of the first host material T₁(H1) in the first emitting layer and a triplet energy of the second host material T₁(H2) in the second emitting layer satisfy the Numerical Formula 1A below, and at least one organic layer included in a hole transporting zone contains a third compound represented by the formula (EB1) below:

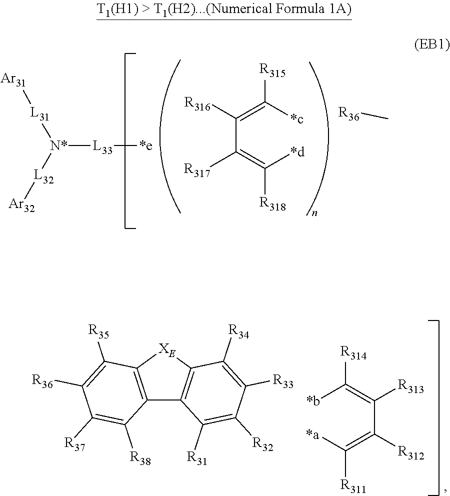

### TECHNICAL FIELD

The present invention relates to an organic electroluminescence device and an electronic device.

### BACKGROUND ART

An organic electroluminescence device (hereinafter, occasionally referred to as “organic EL device”) has found its application in a full-color display for mobile phones, televisions, and the like. When voltage is applied to an organic EL device, holes are injected from an anode and electrons are injected from a cathode into an emitting layer. The injected holes and electrons are recombined in the emitting layer to form excitons. Specifically, according to the electron spin statistics theory, singlet excitons and triplet excitons are generated at a ratio of 25%:75%.

The performance of the organic EL device is evaluable in terms of, for instance, luminance, emission wavelength, chromaticity, luminous efficiency, drive voltage, and lifetime. In Patent Literatures 1 and 2, for instance, studies have been made to improve the performance of an organic EL device.

### CITATION LIST

### Patent Literature(S)

Patent Literature 1 International Publication No. WO 2022/009999
Patent Literature 2 JP 2019-161218 A

### SUMMARY OF THE INVENTION

### Problem(s) to be Solved by the Invention

An object of the invention is to provide an organic electroluminescence device having improved luminous efficiency and a long lifetime, and an electronic device including the organic electroluminescence device.

### Means for Solving the Problem(s)

According to an aspect of the invention, there is provided an organic electroluminescence device, including: an anode; a cathode; an emitting zone disposed between the anode and the cathode; and a hole transporting zone disposed between the anode and the emitting zone, in which the emitting zone includes a first emitting layer and a second emitting layer, the first emitting layer contains a first host material and a first luminescent compound that emits light having a maximum peak wavelength of 500 nm or less, the second emitting layer contains a second host material and a second luminescent compound that emits light having a maximum peak wavelength of 500 nm or less, the first host material and the second host material are mutually different, a triplet energy of the first host material T₁(H1) and a triplet energy of the second host material T₁(H2) satisfy a relationship of a numerical formula (Numerical Formula 1A) below, the first luminescent compound and the second luminescent compound are mutually the same or different, the hole transporting zone includes one or more organic layers, and at least one of the organic layers contains a third compound represented by a formula (EB1) below.

T₁(H1)>T₁(H2) (Numerical Formula 1A)

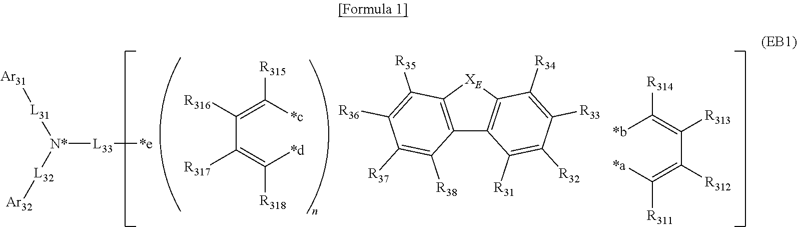

In the formula (EB1):
N* is a central nitrogen atom;
R₃₁ to R₃₈ and R₃₁₁ to R₃₁₈ are each independently a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms;
R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different;
when two or more R₉₀₇ are present, the two or more R₉₀₇ are mutually the same or different;
n is 0 or 1;
when n is 0, one of R₃₁ and R₃₂, one of R₃₂ and R₃₃, or one of R₃₃ and R₃₄ is a single bond with *a, and the other of R₃₁ and R₃₂, the other of R₃₂ and R₃₃, or the other of R₃₃ and R₃₄ is a single bond with *b; and one selected from R₃₁ to R₃₄ being neither the single bond with *a nor the single bond with *b, R₃₅ to R₃₈, and R₃₁₁ to R₃₁₄ is a single bond with *e;
when n is 1, one of R₃₁ and R₃₂, one of R₃₂ and R₃₃, or one of R₃₃ and R₃₄ is a single bond with *a, and the other of R₃₁ and R₃₂, the other of R₃₂ and R₃₃, or the other of R₃₃ and R₃₄ is a single bond with *b; one of R₃₅ and R₃₆, one of R₃₆ and R₃₇, or one of R₃₇ and R₃₈ is a single bond with *c, and the other of R₃₅ and R₃₆, the other of R₃₆ and R₃₇, or the other of R₃₇ and R₃₈ is a single bond with *d; and one selected from R₃₁ to R₃₄ being neither the single bond with *a nor the single bond with *b, R₃₅ to R₃₈ being neither the single bond with *c nor the single bond with *d, R₃₁₁ to R₃₁₄, and R₃₁₅ to R₃₁₈ is a single bond with *e;
X_{E} is an oxygen atom or a sulfur atom;
Ar₃₁ and Ar₃₂ are each independently a substituted or unsubstituted aryl group having 6 to 30 ring carbon atoms, or a substituted or unsubstituted heterocyclic group having 5 to 30 ring atoms; and
L₃₁ to L₃₃ are each independently a single bond, a substituted or unsubstituted arylene group having 6 to 30 ring carbon atoms, or a substituted or unsubstituted divalent heterocyclic group having 5 to 30 ring atoms.

According to another aspect of the invention, there is provided an electronic device including the organic electroluminescence device according to the aspect of the invention.

According to the aspects of the invention, there are provided an organic electroluminescence device having improved luminous efficiency and a long lifetime, and an electronic device including the organic electroluminescence device.

### BRIEF EXPLANATION OF DRAWINGS

FIG. 1 schematically illustrates an exemplary arrangement of an organic electroluminescence device according to an exemplary embodiment of the invention.

FIG. 2 schematically illustrates another exemplary arrangement of the organic electroluminescence device according to the exemplary embodiment of the invention.

### DESCRIPTION OF EMBODIMENT(S)

### Definitions

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Herein, the ring carbon atoms refer to the number of carbon atoms among atoms forming a ring of a compound (e.g., a monocyclic compound, fused-ring compound, cross-linking compound, carbon ring compound, and heterocyclic compound) in which the atoms are bonded to each other to form the ring.

When the ring is substituted by a substituent(s), carbon atom(s) contained in the substituent(s) is not counted in the ring carbon atoms. Unless otherwise specified, the same applies to the “ring carbon atoms” described later. For instance, a benzene ring has 6 ring carbon atoms, a naphthalene ring has 10 ring carbon atoms, a pyridine ring has 5 ring carbon atoms, and a furan ring has 4 ring carbon atoms. Further, for instance, 9,9-diphenylfluorenyl group has 13 ring carbon atoms and 9,9′-spirobifluorenyl group has 25 ring carbon atoms.

When a benzene ring is substituted by a substituent in a form of, for instance, an alkyl group, the number of carbon atoms of the alkyl group is not counted in the number of the ring carbon atoms of the benzene ring. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, cross-linking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent is not counted as the pyridine ring atoms. Accordingly, a pyridine ring bonded to a hydrogen atom(s) or a substituent(s) has 6 ring atoms. For instance, the hydrogen atom(s) bonded to carbon atom(s) of a quinazoline ring or the atoms forming a substituent are not counted as the quinazoline ring atoms. Accordingly, a quinazoline ring bonded to hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and does not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

Substituent Mentioned Herein Substituent mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B). (Herein, an unsubstituted aryl group refers to an “unsubstituted aryl group” in a “substituted or unsubstituted aryl group”, and a substituted aryl group refers to a “substituted aryl group” in a “substituted or unsubstituted aryl group.”) A simply termed “aryl group” herein includes both of an “unsubstituted aryl group” and a “substituted aryl group”.

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below, and a group derived by further substituting a hydrogen atom of a substituent of the “substituted aryl group” in the specific example group G1B below.

### Unsubstituted Aryl Group (Specific Example Group G1A):

a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, perylenyl group, and monovalent aryl group derived by removing one hydrogen atom from cyclic structures represented by formulae (TEMP-1) to (TEMP-15) below.

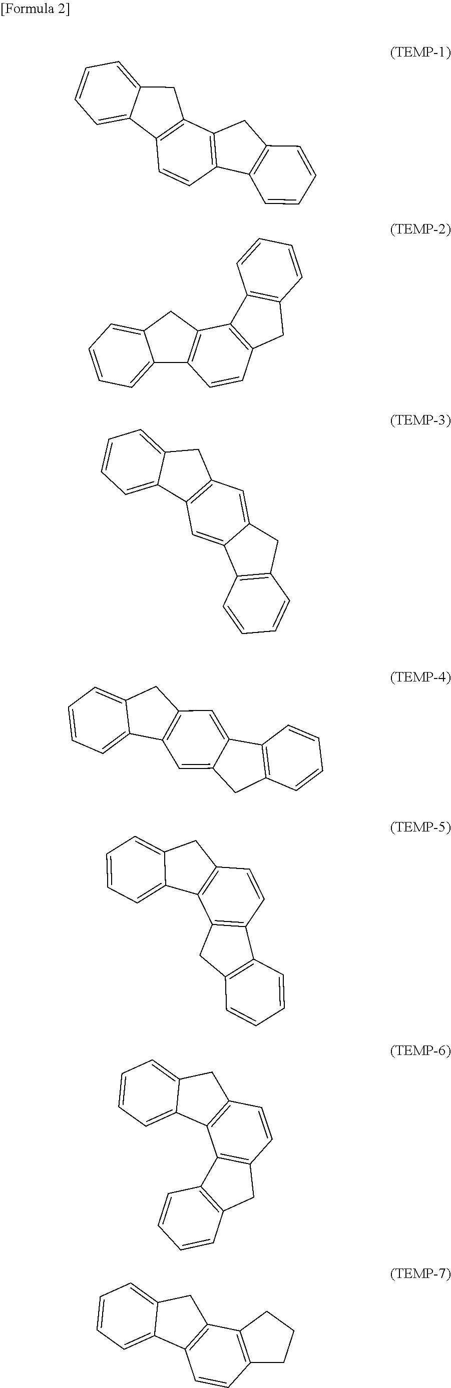

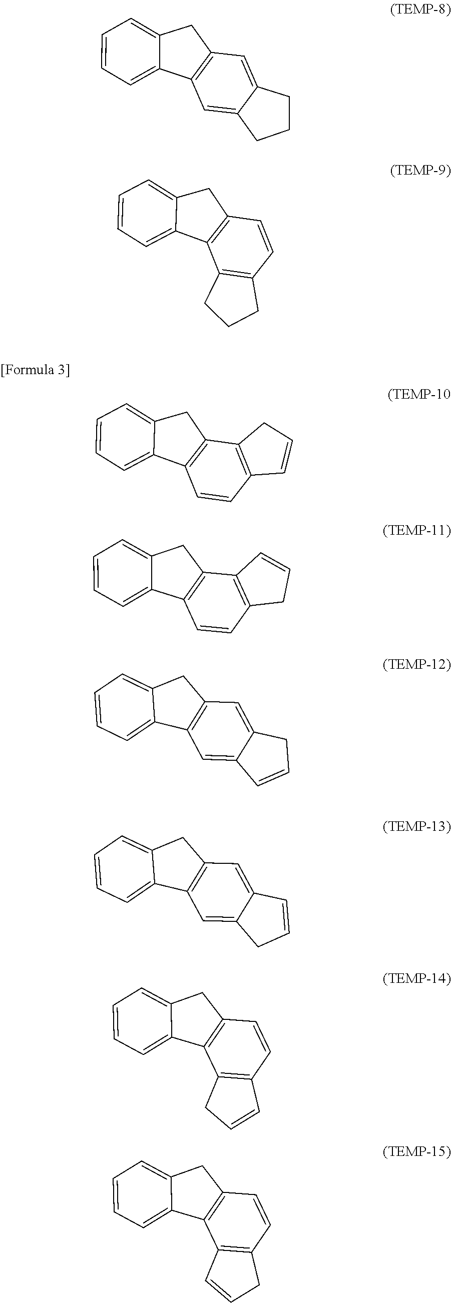

### Substituted Aryl Group (Specific Example Group G1B):

an o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethylfluorenyl group, 9,9-diphenylfluorenyl group, 9,9-bis(4-methylphenyl)fluorenyl group, 9,9-bis(4-isopropylphenyl)fluorenyl group, 9,9-bis(4-t-butylphenyl)fluorenyl group, cyanophenyl group, triphenylsilylphenyl group, trimethylsilylphenyl group, phenylnaphthyl group, naphthylphenyl group, and group derived by substituting at least one hydrogen atom of a monovalent group derived from one of the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one heteroatom in the ring atoms. Specific examples of the heteroatom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B) below. (Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of an “unsubstituted heterocyclic group” and a “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below, and a group derived by further substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

### Unsubstituted Heterocyclic Groups Including Nitrogen Atom (Specific Example Group G2A1):

a pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, pyridyl group, pyridazynyl group, pyrimidinyl group, pyrazinyl group, triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

### Unsubstituted Heterocyclic Groups Including Oxygen Atom (Specific Example Group G2A2):

a furyl group, oxazolyl group, isoxazolyl group, oxadiazolyl group, xanthenyl group, benzofuranyl group, isobenzofuranyl group, dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

### Unsubstituted Heterocyclic Groups Including Sulfur Atom (Specific Example Group G2A3):

a thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).

Monovalent Heterocyclic Groups Derived by Removing One Hydrogen Atom from Cyclic Structures Represented by Formulae (TEMP-16) to (TEMP-33) (Specific Example Group G2A4):

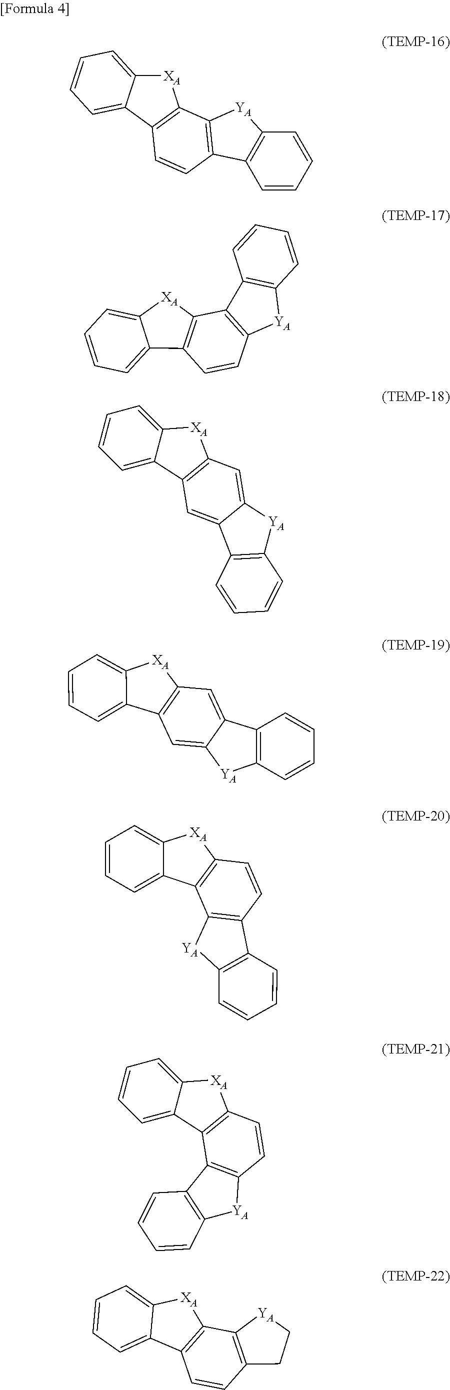

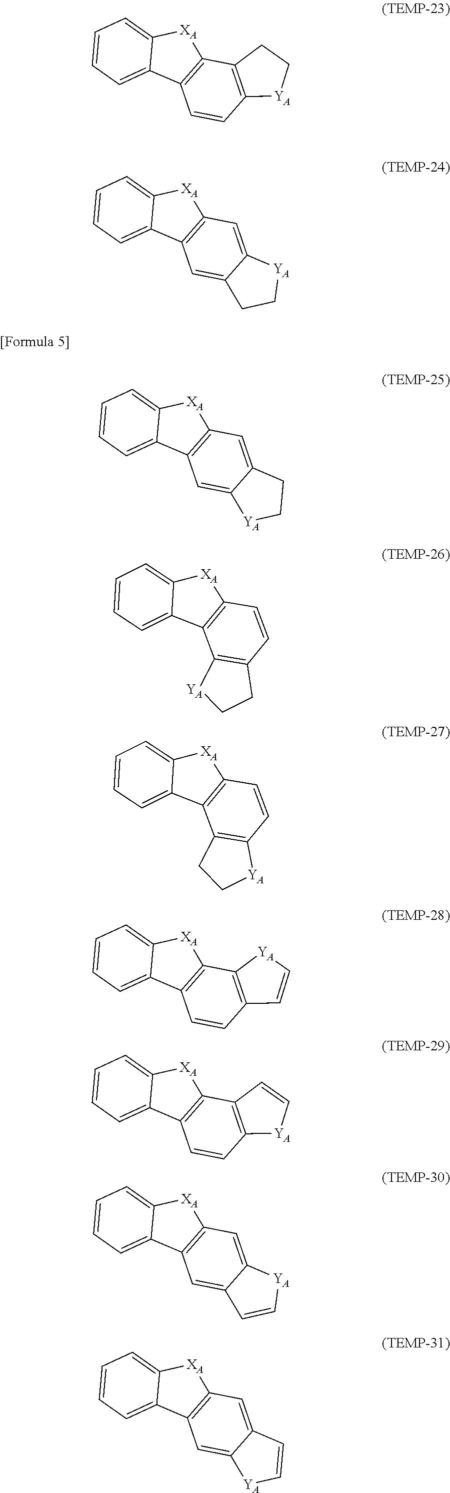

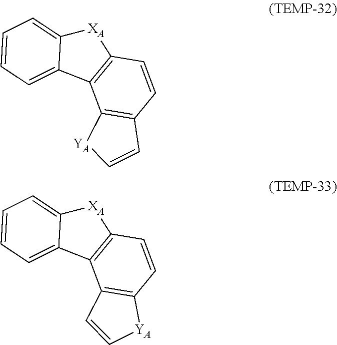

In the formulae (TEMP-16) to (TEMP-33), XA and YA are each independently an oxygen atom, a sulfur atom, NH or CH₂, with a proviso that at least one of XA or YA is an oxygen atom, a sulfur atom, or NH.

When at least one of XA or YA in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH or CH₂.

### Substituted Heterocyclic Groups Including Nitrogen Atom (Specific Example Group G2B1):

a (9-phenyl)carbazolyl group, (9-biphenylyl)carbazolyl group, (9-phenyl)phenylcarbazolyl group, (9-naphthyl)carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, phenylquinazolinyl group, and biphenylquinazolinyl group.

### Substituted Heterocyclic Groups Including Oxygen Atom (Specific Example Group G2B2):

a phenyldibenzofuranyl group, methyldibenzofuranyl group, t-butyldibenzofuranyl group, and monovalent residue of spiro[9H-xanthene-9,9′-[9H]fluorene].

### Substituted Heterocyclic Groups Including Sulfur Atom (Specific Example Group G2B3):

a phenyldibenzothiophenyl group, methyldibenzothiophenyl group, t-butyldibenzothiophenyl group, and monovalent residue of spiro[9H-thioxanthene-9,9′-[9H]fluorene].

Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of XA or YA in a form of NH, and a hydrogen atom of one of XA and YA in a form of a methylene group (CH₂).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B) below. (Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”) A simply termed “alkyl group” herein includes both of an “unsubstituted alkyl group” and a “substituted alkyl group”.

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B, and a group derived by further substituting a hydrogen atom of a substituent of the “substituted alkyl group” in the specific example group G3B.

### Unsubstituted Alkyl Group (Specific Example Group G3A):

a methyl group, ethyl group, n-propyl group, isopropyl group, n-butyl group, isobutyl group, s-butyl group, and t-butyl group.

### Substituted Alkyl Group (Specific Example Group G3B):

a heptafluoropropyl group (including isomer thereof), pentafluoroethyl group, 2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). (Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”) A simply termed “alkenyl group” herein includes both of an “unsubstituted alkenyl group” and a “substituted alkenyl group”.

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

### Unsubstituted Alkenyl Group (Specific Example Group G4A):

a vinyl group, allyl group, 1-butenyl group, 2-butenyl group, and 3-butenyl group.

### Substituted Alkenyl Group (Specific Example Group G4B):

a 1,3-butanedienyl group, 1-methylvinyl group, 1-methylallyl group, 1,1-dimethylallyl group, 2-methylallyl group, and 1,2-dimethylallyl group.

### Substituted or Unsubstituted Alkynyl Group

Specific examples (specific example group G5) of the “substituted or unsubstituted alkynyl group” mentioned herein include unsubstituted alkynyl groups (specific example group G5A) below. (Herein, an unsubstituted alkynyl group refers to an “unsubstituted alkynyl group” in a “substituted or unsubstituted alkynyl group.”) A simply termed “alkynyl group” herein includes both of “unsubstituted alkynyl group” and “substituted alkynyl group”.

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

Unsubstituted Alkynyl Group (Specific Example Group G5A): an ethynyl group.

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B) below. (Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.”) A simply termed “cycloalkyl group” herein includes both of “unsubstituted cycloalkyl group” and “substituted cycloalkyl group”.

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

### Unsubstituted Cycloalkyl Group (Specific Example Group G6A):

a cyclopropyl group, cyclobutyl group, cyclopentyl group, cyclohexyl group, 1-adamantyl group, 2-adamantyl group, 1-norbornyl group, and 2-norbornyl group.

### Substituted Cycloalkyl Group (Specific Example Group G6B):

a 4-methylcyclohexyl group.

Group Represented by —Si(R₉₀₁)(R₉₀₂)(R₉₀₃)

Specific examples (specific example group G7) of the group represented herein by —Si(R₉₀₁)(R₉₀₂)(R₉₀₃) include: —Si(G1)(G1)(G1); —Si(G1)(G2)(G2); —Si(G1)(G1)(G2); —Si(G2)(G2)(G2); —Si(G3)(G3)(G3); and —Si(G6)(G6)(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —Si(G1)(G1)(G1) are mutually the same or different;
a plurality of G2 in —Si(G1)(G2)(G2) are mutually the same or different;
a plurality of G1 in —Si(G1)(G1)(G2) are mutually the same or different;
a plurality of G2 in —Si(G2)(G2)(G2) are mutually the same or different;
a plurality of G3 in —Si(G3)(G3)(G3) are mutually the same or different; and
a plurality of G6 in —Si(G6)(G6)(G6) are mutually the same or different.

### Group Represented by —O—(R₉₀₄)

Specific examples (specific example group G8) of a group represented by —O—(R₉₀₄) herein include: —O(G1); —O(G2); —O(G3); and —O(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.

### Group Represented by —S—(R₉₀₅)

Specific examples (specific example group G9) of a group represented herein by —S—(R₉₀₅) include: —S(G1); —S(G2); —S(G3); and —S(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.
   Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6)(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —N(G1)(G1) are mutually the same or different;
a plurality of G2 in —N(G2)(G2) are mutually the same or different;
a plurality of G3 in —N(G3)(G3) are mutually the same or different; and
a plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent. Specific examples of the “unsubstituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50, preferably 1 to 30, and more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent. Specific examples of the “unsubstituted haloalkyl group” include a group derived by substituting at least one hydrogen atom of the “alkyl group” (specific example group G3) with a halogen atom. The haloalkyl group is occasionally referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “trialkylsilyl group” mentioned herein include a group represented by —Si(G3)(G3)(G3), G3 being the “substituted or unsubstituted alkyl group” in the specific example group G3. A plurality of G3 in —Si(G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by -(G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl)carbazolyl group ((9-phenyl)carbazole-1-yl group, (9-phenyl)carbazole-2-yl group, (9-phenyl)carbazole-3-yl group, or (9-phenyl)carbazole-4-yl group), (9-biphenylyl)carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

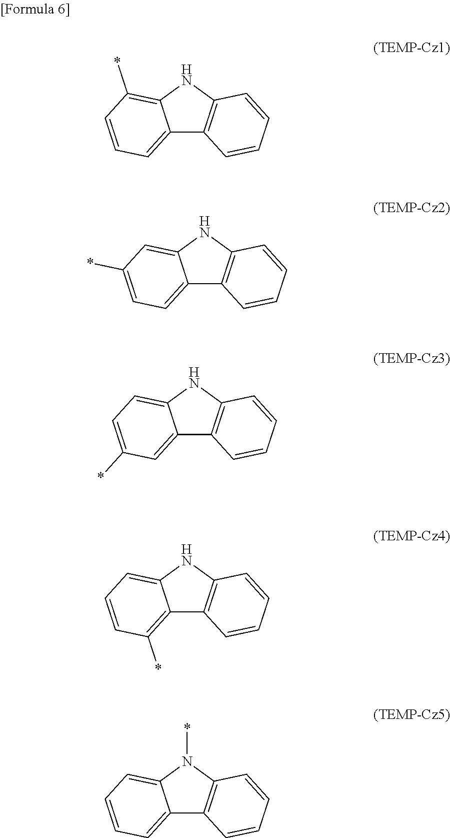 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

The (9-phenyl)carbazolyl group mentioned herein is, unless otherwise specified herein, specifically a group represented by one of formulae below.

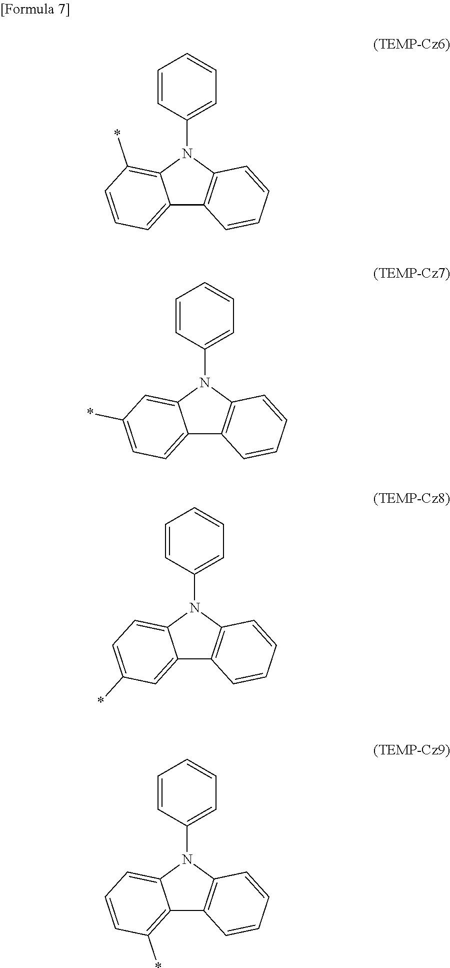 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

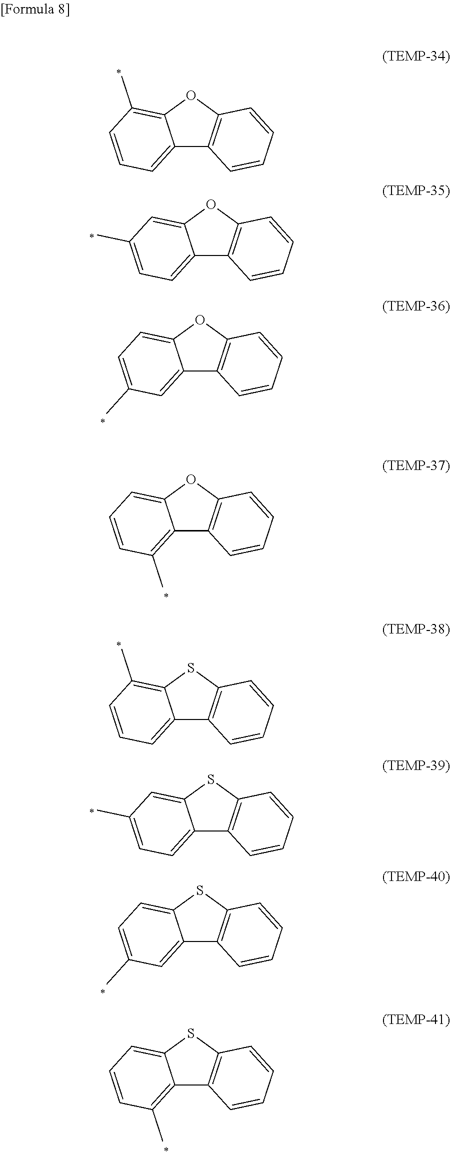

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocycle of the “substituted or unsubstituted heterocyclic group.” Specific examples of the “substituted or unsubstituted divalent heterocyclic group” (specific example group G13) include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl chain of the “substituted or unsubstituted alkyl group.” Specific examples of the “substituted or unsubstituted alkylene group” (specific example group G14) include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

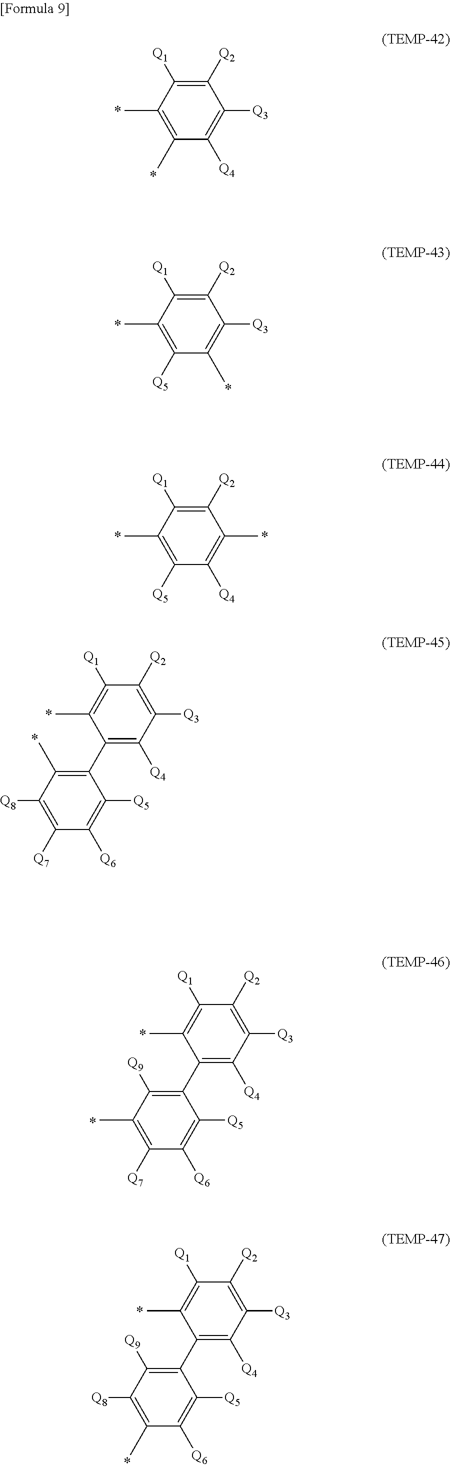

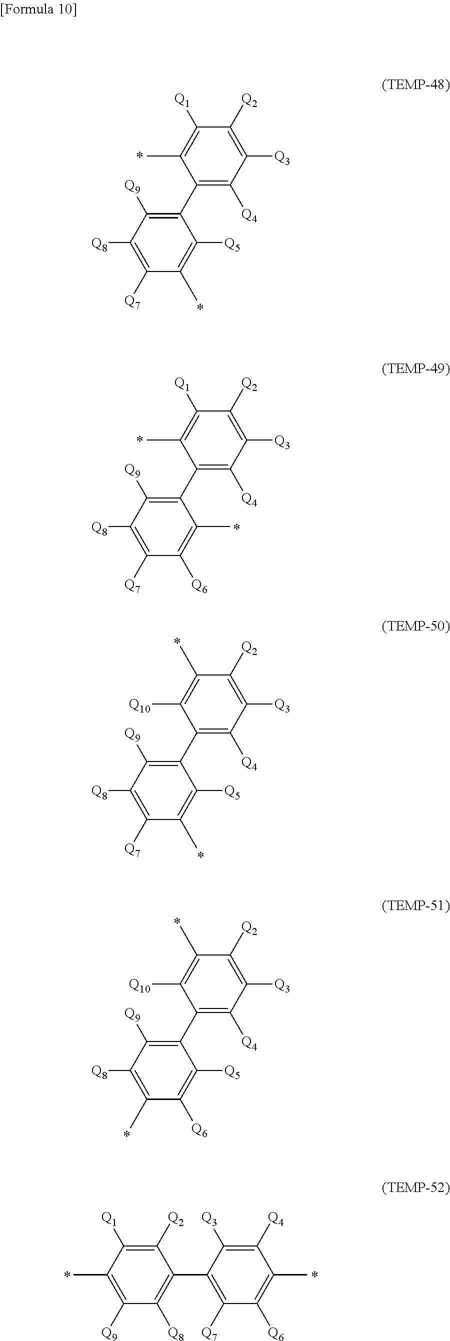

In the formulae (TEMP-42) to (TEMP-52), Q₁ to Q₁₀ are each independently a hydrogen atom or a substituent.

In the formulae (TEMP-42) to (TEMP-52), * represents a bonding position.

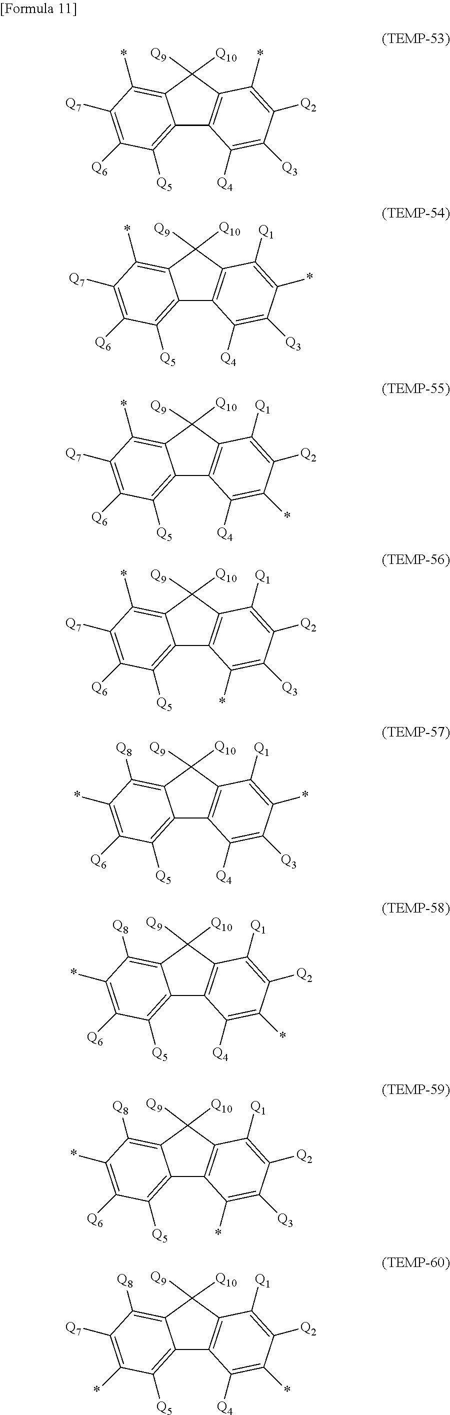

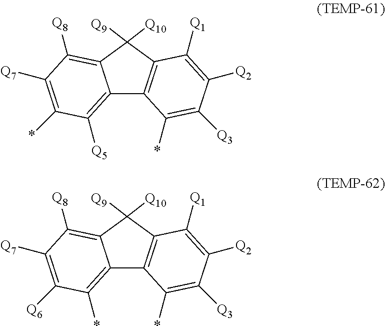

In the formulae (TEMP-53) to (TEMP-62), Q₁ to Q₁₀ are each independently a hydrogen atom or a substituent.

In the formulae, Q₉ and Q₁₀ may be mutually bonded through a single bond to form a ring.

In the formulae (TEMP-53) to (TEMP-62), * represents a bonding position.

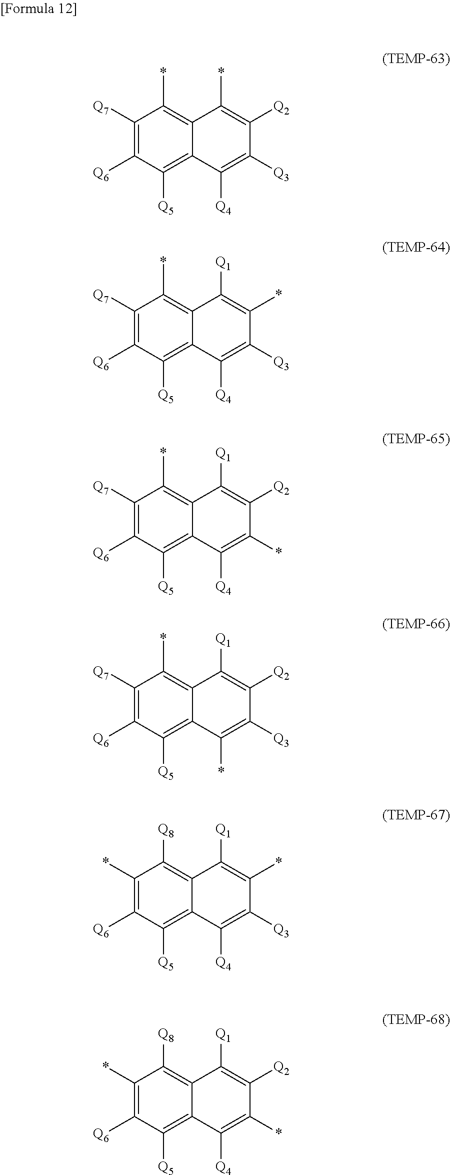

In the formulae (TEMP-63) to (TEMP-68), Q₁ to Q₈ are each independently a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

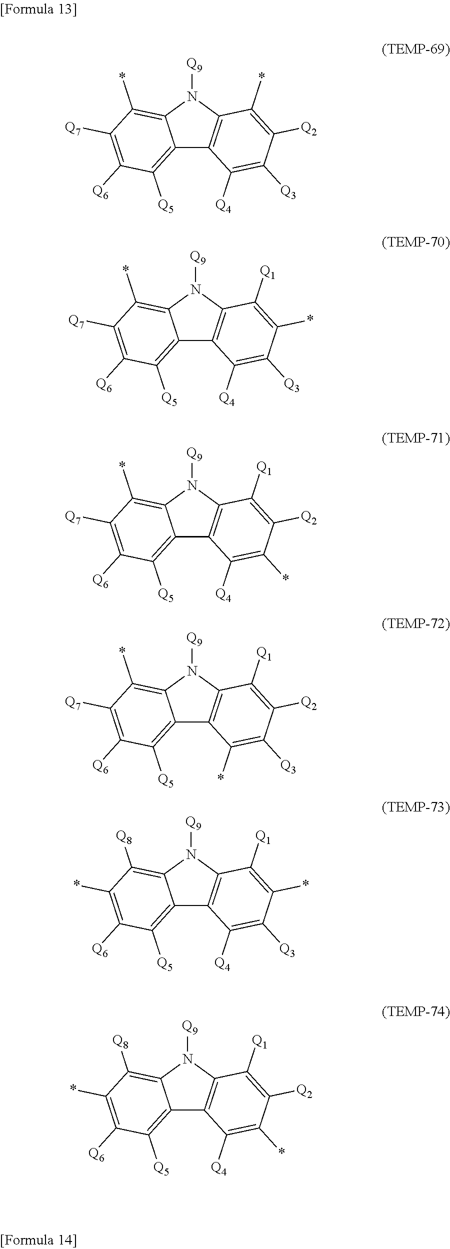

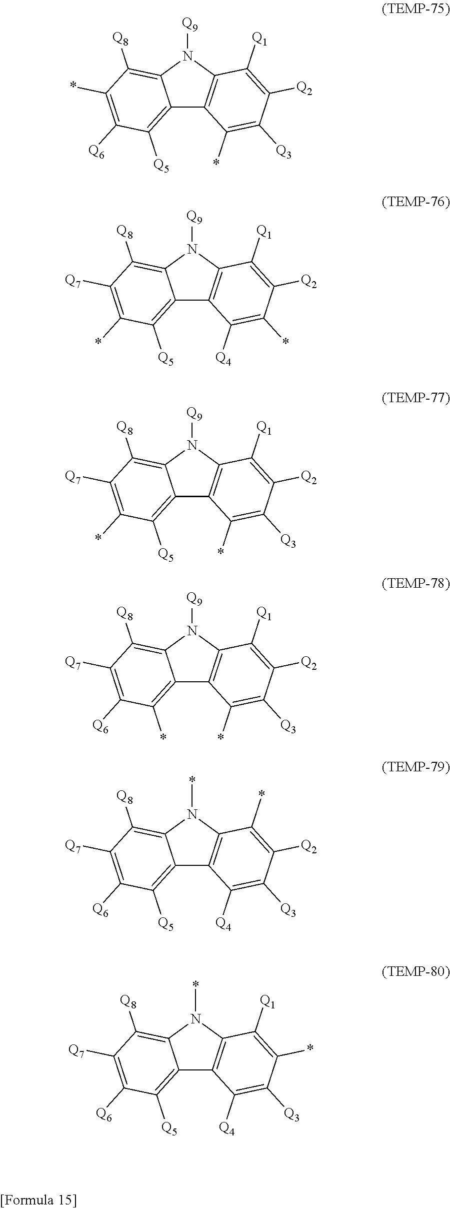

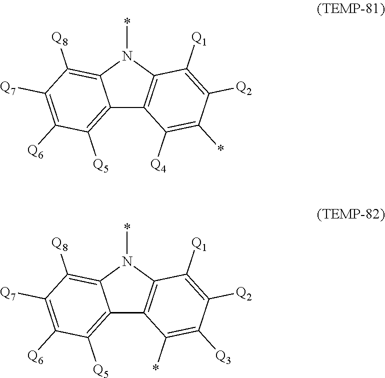

In the formulae (TEMP-69) to (TEMP-82), Q₁ to Q₉ are each independently a hydrogen atom or a substituent.

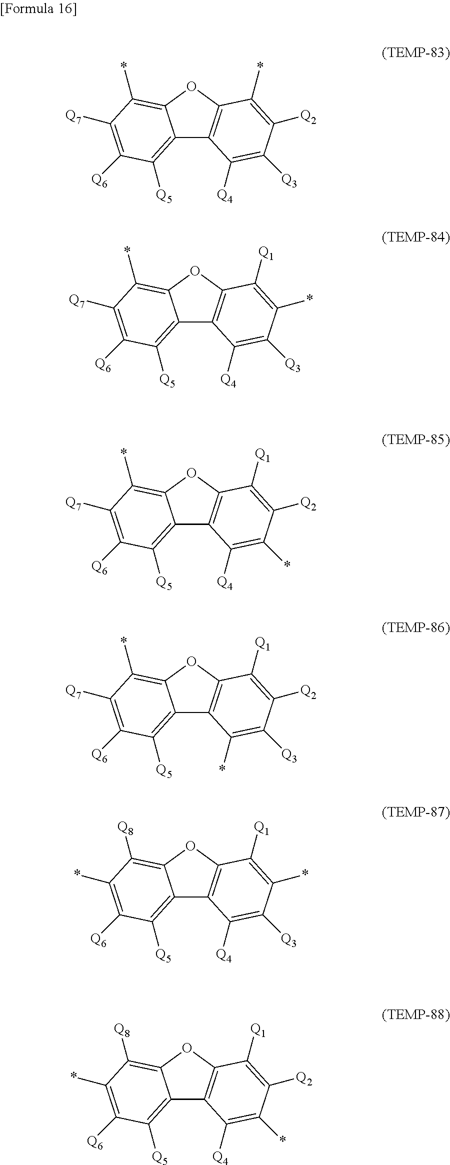

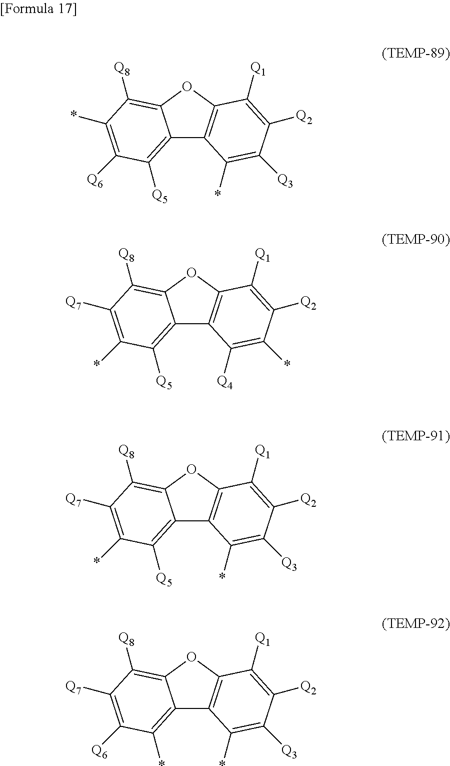

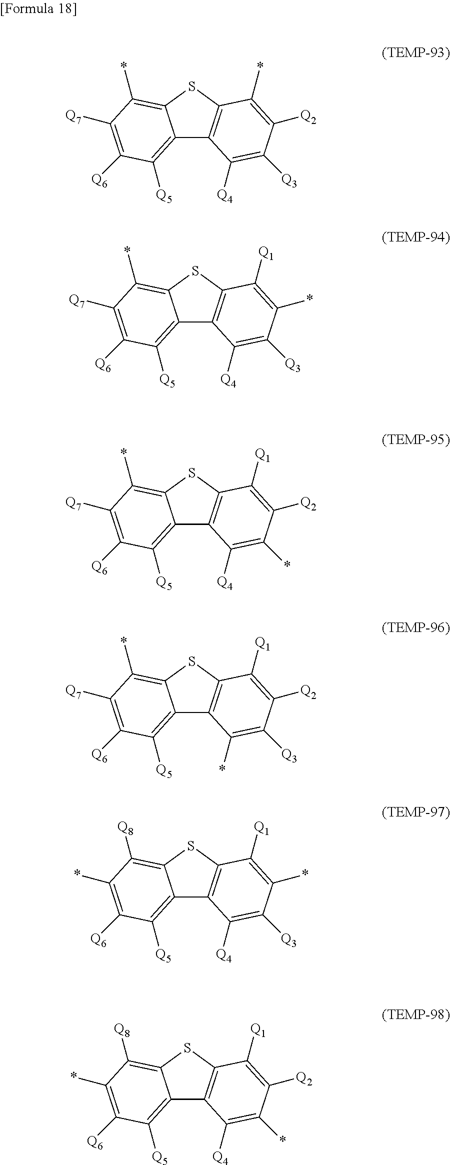

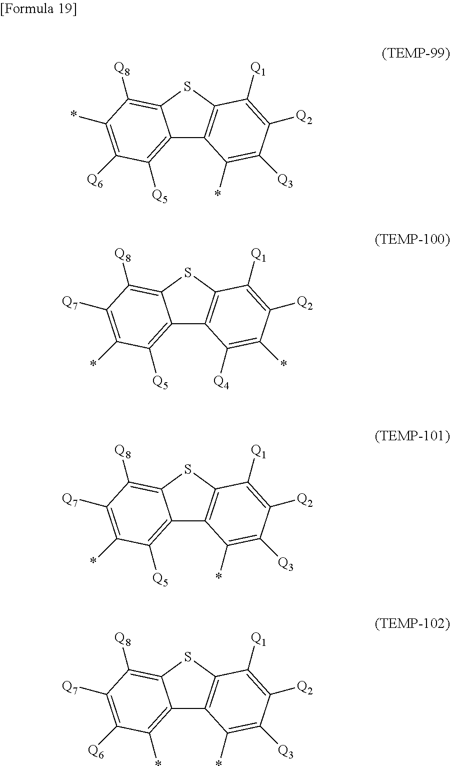

In the formulae (TEMP-83) to (TEMP-102), Q₁ to Q₈ are each independently a hydrogen atom or a substituent.

The substituent mentioned herein has been described above.

### Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring”, “at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

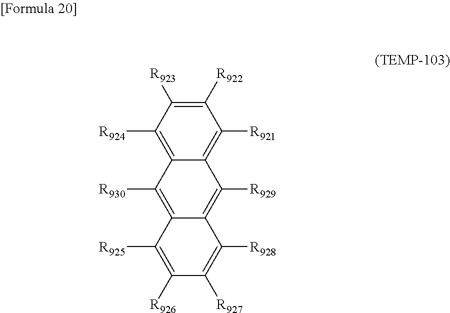

For instance, when “at least one combination of adjacent two or more of R₉₂₁ to R₉₃₀ are mutually bonded to form a ring,” the combination of adjacent ones of R₉₂₁ to R₉₃₀ (i.e. the combination at issue) is a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, or a combination of R₉₂₉ and R₉₂₁.

The term “at least one combination” means that two or more of the above combinations of adjacent two or more of R₉₂₁ to R₉₃₀ may simultaneously form rings. For instance, when R₉₂₁ and R₉₂₂ are mutually bonded to form a ring Q_{A} and R₉₂₅ and R₉₂₆ are simultaneously mutually bonded to form a ring Q_{B}, the anthracene compound represented by the formula (TEMP-103) is represented by a formula (TEMP-104) below.

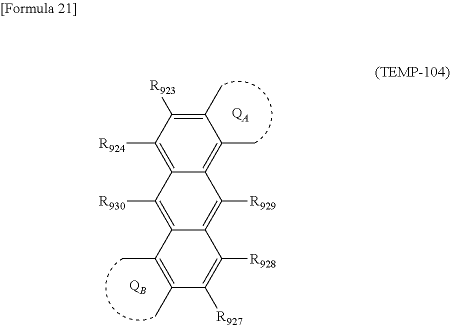

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂ and R₉₂₃ are mutually bonded to form a ring Q_{C}, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring Q_{C} share R₉₂₂.

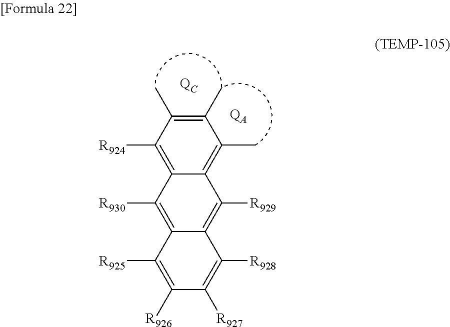

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring Q_{B} formed in the formula (TEMP-104) are each independently a “monocyclic ring” or a “fused ring.” Further, the ring Q_{A} and the ring Q_{C} formed in the formula (TEMP-105) are each a “fused ring.” The ring Q_{A} and the ring Q_{C} in the formula (TEMP-105) are fused to form a fused ring. When the ring Q_{A} in the formula (TEMP-104) is a benzene ring, the ring Q_{A} is a monocyclic ring. When the ring Q_{A} in the formula (TEMP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by terminating a bond of a group in the specific example of the specific example group G1 with a hydrogen atom.

Specific examples of the aromatic heterocycle include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded to R₉₂₁, a carbon atom of the anthracene skeleton bonded to R₉₂₂, and one or more optional atoms. Specifically, when the ring Q_{A} is a monocyclic unsaturated ring formed by R₉₂₁ and R₉₂₂, the ring formed by a carbon atom of the anthracene skeleton bonded to R₉₂₁, a carbon atom of the anthracene skeleton bonded to R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes any other optional element than the carbon atom, the resultant ring is a heterocycle.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

When the “saturated ring” or the “unsaturated ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance of “bonded to form a ring”).

### Substituent for Substituted or Unsubstituted Group

In an exemplary embodiment herein, the substituent for the substituted or unsubstituted group (hereinafter occasionally referred to as an “optional substituent”), is for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms;
R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different; and
when two or more R₉₀₇ are present, the two or more R₉₀₇ are mutually the same or different.

In an exemplary embodiment, the substituent for the substituted or unsubstituted group is a group selected from the group consisting of an alkyl group having 1 to 50 carbon atoms, an aryl group having 6 to 50 ring carbon atoms, and a heterocyclic group having 5 to 50 ring atoms.

In an exemplary embodiment, the substituent for the substituted or unsubstituted group is a group selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted unsaturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represent a range whose lower limit is the value (AA) recited before “to” and whose upper limit is the value (BB) recited after “to.”

Herein, a numerical formula represented by “A≥B” means that the value A is equal to the value B, or the value A is larger than the value B.

Herein, a numerical formula represented by “A≤B” means that the value A is equal to the value B, or the value A is smaller than the value B.

### First Exemplary Embodiment

An organic electroluminescence device according to a first exemplary embodiment includes: an anode; a cathode; an emitting zone disposed between the anode and the cathode; and a hole transporting zone disposed between the anode and the emitting zone, in which the emitting zone includes a first emitting layer and a second emitting layer, the first emitting layer contains a first host material and a first luminescent compound that emits light having a maximum peak wavelength of 500 nm or less, the second emitting layer contains a second host material and a second luminescent compound that emits light having a maximum peak wavelength of 500 nm or less, the first host material and the second host material are mutually different, a triplet energy of the first host material T₁(H1) and a triplet energy of the second host material T₁(H2) satisfy a relationship of a numerical formula (Numerical Formula 1A) below, the first luminescent compound and the second luminescent compound are mutually the same or different, the hole transporting zone includes one or more organic layers, and at least one of the organic layers contains a third compound represented by a formula (EB1) below.

$${T}_{1} ( H 1 ) > {T}_{1} ( H 2 ) \qquad ( Numerical Formula 1 A )$$

According to the exemplary embodiment, an organic EL device having improved luminous efficiency and a long lifetime can be provided.

In the organic EL device according to the exemplary embodiment, the first host material contained in the first emitting layer and the second host material contained in the second emitting layer satisfy the relationship of the numerical formula (Numerical Formula 1A), resulting in improved luminous efficiency compared to an emitting zone including a single emitting layer.

Conventionally, triplet-triplet-annihilation (occasionally referred to as TTA) has been known as a technique for improving the luminous efficiency of the organic electroluminescence device. TTA is a mechanism in which triplet excitons collide with one another to generate singlet excitons. The TTA mechanism is also referred to as a TTF mechanism. TTF is an abbreviation for Triplet-Triplet Fusion.

The TTF phenomenon will be described. Holes injected from an anode and electrons injected from a cathode are recombined in an emitting layer to generate excitons. As for the spin state, as is conventionally known, singlet excitons account for 25% and triplet excitons account for 75%. In a conventionally known fluorescent device, light is emitted when singlet excitons of 25% are relaxed to the ground state. The remaining triplet excitons of 75% are returned to the ground state without emitting light through a thermal deactivation process. Accordingly, the theoretical limit value of the internal quantum efficiency of the conventional fluorescent device is believed to be 25%.

The behavior of triplet excitons generated within an organic substance has been theoretically examined. According to S. M. Bachilo et al. (J. Phys. Chem. A, 104, 7711 (2000)), assuming that high-order excitons such as quintet excitons are quickly returned to triplet excitons, triplet excitons (hereinafter abbreviated as ³A*) collide with one another with an increase in density thereof, whereby a reaction shown by the following formula occurs. In the formula, ¹A represents the ground state and ¹A* represents the lowest singlet excitons.

$${}^{3} {A}^{⋆} + {}^{3} {A}^{⋆} \to {( 4 / 9 )}^{1} A + {( 1 / 9 )}^{1} {A}^{*} + {( 13 / 9 )}^{3} {A}^{*}$$

In other words, 5³A*→4¹A+1A* is satisfied, and it is expected that, among triplet excitons initially generated, which account for 75%, one fifth thereof (i.e., 20%) is changed to singlet excitons. Accordingly, the amount of singlet excitons contributing to emission is 40%, which is a value obtained by adding 15% (75%×(1/5)=15%) to 25%, which is the amount ratio of initially generated singlet excitons. At this time, a ratio of luminous intensity derived from TTF (TTF ratio) relative to the total luminous intensity is 15/40, i.e., 37.5%. Assuming that singlet excitons are generated by collision of initially generated triplet excitons accounting for 75% (i.e., one singlet exciton is generated from two triplet excitons), a significantly high internal quantum efficiency of 62.5% is obtained, which is a value obtained by adding 37.5% (75%×(1/2)=37.5%) to 25% (the amount ratio of initially generated singlet excitons). At this time, the TTF ratio is 37.5/62.5=60%.

In the organic electroluminescence device according to the exemplary embodiment, it is considered that triplet excitons generated by recombination of holes and electrons in the first emitting layer and present on an interface between the first emitting layer and organic layer(s) in direct contact therewith are not likely to be quenched even under the presence of excessive carriers on the interface between the first emitting layer and the organic layer(s). For instance, the presence of a recombination region locally on an interface between the first emitting layer and a hole transporting layer or an electron blocking layer is considered to cause quenching by excessive electrons. Meanwhile, the presence of a recombination region locally on an interface between the first emitting layer and an electron transporting layer or a hole blocking layer is considered to cause quenching by excessive holes.

The organic electroluminescence device according to the exemplary embodiment includes at least two emitting layers (i.e., the first emitting layer and the second emitting layer) satisfying a predetermined relationship. The triplet energy of the first host material T₁(H1) in the first emitting layer and the triplet energy of the second host material T₁(H2) in the second emitting layer satisfy the relationship of the numerical formula (Numerical Formula 1A).

By including the first emitting layer and the second emitting layer so as to satisfy the relationship of the numerical formula (Numerical Formula 1A), triplet excitons generated in the first emitting layer can transfer to the second emitting layer without being quenched by excessive carriers and be inhibited from back-transferring from the second emitting layer to the first emitting layer. Consequently, the second emitting layer exhibits the TTF mechanism to effectively generate singlet excitons, thereby improving the luminous efficiency.

Accordingly, the organic electroluminescence device includes, as different regions, the first emitting layer mainly generating triplet excitons and the second emitting layer mainly exhibiting the TTF mechanism using triplet excitons having transferred from the first emitting layer, and has a difference in triplet energy provided by using a compound having a smaller triplet energy than that of the first host material in the first emitting layer as the second host material in the second emitting layer. The luminous efficiency is thus improved.

In the organic EL device according to the exemplary embodiment, the triplet energy of the first host material T₁(H1) and the triplet energy of the second host material T₁(H2) preferably satisfy a relationship of a numerical formula (Numerical Formula 1B) below.

$${T}_{1} ( H 1 ) - {T}_{1} ( H 2 ) > 0.03 eV \qquad ( Numerical Formula 1 B )$$

The third compound represented by a formula (EB1) below contained in the organic EL device according to the exemplary embodiment effectively traps electrons. The organic layer included in the hole transporting zone located on the anode side with respect to the emitting zone contains the third compound represented by the formula (EB1) below, thus facilitating the recombination of carriers in the emitting layer located on the hole transporting zone side to allow the organic EL device to emit light with high efficiency for a long time.

In addition, the third compound represented by the formula (EB1) has high excitation resistance, which allows the organic EL device containing the third compound to have a long lifetime.

### Hole Transporting Zone

The hole transporting zone is provided between the anode and the emitting zone.

The hole transporting zone includes one or more organic layers. At least one of the organic layers included in the hole transporting zone contains the third compound represented by the formula (EB1).

In an exemplary arrangement of the organic EL device of the exemplary embodiment, the hole transporting zone includes two or more organic layers. In an exemplary arrangement of the organic EL device of the exemplary embodiment, at least one of two or more organic layers included in the hole transporting zone contains the third compound represented by the formula (EB1).

In an exemplary arrangement of the organic EL device according to the exemplary embodiment, the hole transporting zone includes a hole injecting layer and a hole transporting layer.

In an exemplary arrangement of the organic EL device according to the exemplary embodiment, when the hole transporting zone includes two organic layers that are the hole injecting layer and the hole transporting layer, the hole transporting layer contains the third compound represented by the formula (EB1).

In an exemplary arrangement of the organic EL device according to the exemplary embodiment, the hole transporting layer is in direct contact with the emitting layer in the emitting zone.

In an exemplary arrangement of the organic EL device of the exemplary embodiment, the hole transporting zone includes three or more organic layers. In an exemplary arrangement of the organic EL device of the exemplary embodiment, at least one of three or more organic layers included in the hole transporting zone contains the third compound represented by the formula (EB1).

In an exemplary arrangement of the organic EL device according to the exemplary embodiment, the hole transporting zone includes a hole injecting layer, a hole transporting layer, and an electron blocking layer.

In an exemplary arrangement of the organic EL device according to the exemplary embodiment, when the hole transporting zone includes three organic layers that are the hole injecting layer, the hole transporting layer, and the electron blocking layer, the electron blocking layer contains the third compound represented by the formula (EB1).

In an exemplary arrangement of the organic EL device according to the exemplary embodiment, the electron blocking layer is preferably in direct contact with the emitting layer in the emitting zone.

In an exemplary arrangement of the organic EL device of the exemplary embodiment, one of the organic layers included in the hole transporting zone and disposed closest to the cathode contains the third compound represented by the formula (EB1).

In an exemplary arrangement of the organic EL device according to the exemplary embodiment, one of the organic layers included in the hole transporting zone and being in direct contact with the emitting layer in the emitting zone contains the third compound represented by the formula (EB1).

### Third Compound

The third compound in the exemplary embodiment will be described below.

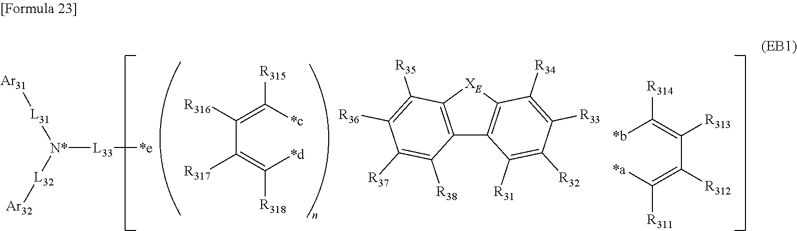

In the formula (EB1):
N* is a central nitrogen atom,
R₃₁ to R₃₈ and R₃₁₁ to R₃₁₈ are each independently a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms;
R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different;
when two or more R₉₀₇ are present, the two or more R₉₀₇ are mutually the same or different;
n is 0 or 1;
when n is 0, one of R₃₁ and R₃₂, one of R₃₂ and R₃₃, or one of R₃₃ and R₃₄ is a single bond with *a, and the other of R₃₁ and R₃₂, the other of R₃₂ and R₃₃, or the other of R₃₃ and R₃₄ is a single bond with *b; and one selected from R₃₁ to R₃₄ being neither the single bond with *a nor the single bond with *b, R₃₅ to R₃₈, and R₃₁₁ to R₃₁₄ is a single bond with *e;
when n is 1, one of R₃₁ and R₃₂, one of R₃₂ and R₃₃, or one of R₃₃ and R₃₄ is a single bond with *a, and the other of R₃₁ and R₃₂, the other of R₃₂ and R₃₃, or the other of R₃₃ and R₃₄ is a single bond with *b; one of R₃₅ and R₃₆, one of R₃₆ and R₃₇, or one of R₃₇ and R₃₈ is a single bond with *c, and the other of R₃₅ and R₃₆, the other of R₃₆ and R₃₇, or the other of R₃₇ and R₃₈ is a single bond with *d; and one selected from R₃₁ to R₃₄ being neither the single bond with *a nor the single bond with *b, R₃₅ to R₃₈ being neither the single bond with *c nor the single bond with *d, R₃₁₁ to R₃₁₄, and R₃₁₅ to R₃₁₈ is a single bond with *e;
X_{E} is an oxygen atom or a sulfur atom;
Ar₃₁ and Ar₃₂ are each independently a substituted or unsubstituted aryl group having 6 to 30 ring carbon atoms, or a substituted or unsubstituted heterocyclic group having 5 to 30 ring atoms; and
L₃₁ to L₃₃ are each independently a single bond, a substituted or unsubstituted arylene group having 6 to 30 ring carbon atoms, or a substituted or unsubstituted divalent heterocyclic group having 5 to 30 ring atoms.

In the compound according to the exemplary embodiment, “one of R₃₁ and R₃₂, one of R₃₂ and R₃₃, or one of R₃₃ and R₃₄ is a single bond with *a, and the other of R₃₁ and R₃₂, the other of R₃₂ and R₃₃, or the other of R₃₃ and R₃₄ is a single bond with *b” means that: one of R₃₁ and R₃₂ is a single bond with *a and the other of R₃₁ and R₃₂ is a single bond with *b; one of R₃₂ and R₃₃ is a single bond with *a and the other of R₃₂ and R₃₃ is a single bond with *b; or one of R₃₃ and R₃₄ is a single bond with *a and the other of R₃₃ and R₃₄ is a single bond with *b.

In the compound according to the exemplary embodiment, “one of R₃₅ and R₃₆, one of R₃₆ and R₃₇, or one of R₃₇ and R₃₈ is a single bond with *c, and the other of R₃₅ and R₃₆, the other of R₃₆ and R₃₇, or the other of R₃₇ and R₃₈ is a single bond with *d” means that: one of R₃₅ and R₃₆ is a single bond with *c and the other of R₃₅ and R₃₆ is a single bond with *d; one of R₃₆ and R₃₇ is a single bond with *c and the other of R₃₆ and R₃₇ is a single bond with *d; or one of R₃₇ and R₃₈ is a single bond with *c and the other of R₃₇ and R₃₈ is a single bond with *d.

In an exemplary arrangement of the organic EL device of the exemplary embodiment, R₃₈ is a single bond with *e.

In an exemplary arrangement of the organic EL device of the exemplary embodiment, the third compound represented by the formula (EB1) is a compound represented by any of formulae (EB11) to (EB13) below.

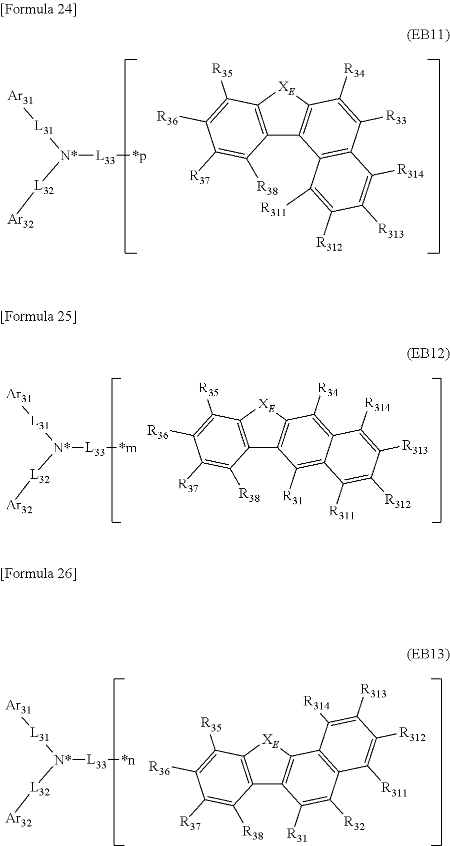

In the formulae (EB11) to (EB13):
N*, R₃₁ to R₃₈, R₃₁₁ to R₃₁₄, X_{E}, Ar₃₁, Ar₃₂, and L₃₁ to L₃₃ respectively represent the same as those defined in the formula (EB1);
when the compound represented by the formula (EB1) is a compound represented by the formula (EB11), one selected from R₃₃ to R₃₈ and R₃₁₁ to R₃₁₄ is a single bond with *p;
when the compound represented by the formula (EB1) is a compound represented by the formula (EB12), one selected from R₃₁, R₃₄ to R₃₈, and R₃₁₁ to R₃₁₄ is a single bond with *m; and
when the compound represented by the formula (EB1) is a compound represented by the formula (EB13), one selected from R₃₁, R₃₂, R₃₅ to R₃₈, and R₃₁₁ to R₃₁₄ is a single bond with *n.

In an exemplary arrangement of the organic EL device of the exemplary embodiment, the compound represented by the formula (EB1) is a compound represented by the formula (EB13).

In an exemplary arrangement of the organic EL device of the exemplary embodiment, R₃₁, R₃₅, R₃₇, R₃₈, or R₃₁₁ in the formula (EB11), (EB12), or (EB13) is a single bond with *p, *m, or *n.

In an exemplary arrangement of the organic EL device of the exemplary embodiment, R₃₈ in the formula (EB11), (EB12), or (EB13) is a single bond with *p, *m, or *n.

In an exemplary arrangement of the organic EL device of the exemplary embodiment, the compound represented by the formula (EB1) is a compound represented by a formula (EB131) below.

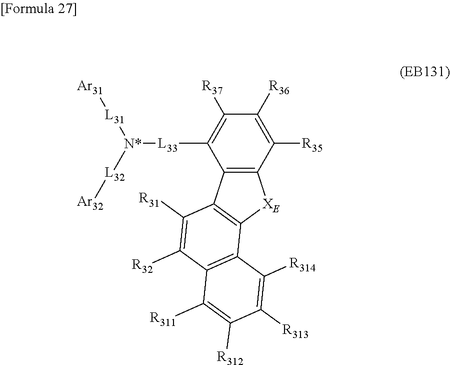

In the formula (EB131), N*, R₃₁, R₃₂, R₃₅ to R₃₇, R₃₁₁ to R₃₁₄, X_{E}, Ar₃₁, Ar₃₂, and L₃₁ to L₃₃ respectively represent the same as those defined in the formula (EB1).

In an exemplary arrangement of the organic EL device of the exemplary embodiment, L₃₃ is a substituted or unsubstituted phenylene group.

In an exemplary arrangement of the organic EL device of the exemplary embodiment, L₃₃ is an unsubstituted phenylene group.

In an exemplary arrangement of the organic EL device of the exemplary embodiment, L₃₃ is a substituted or unsubstituted o-phenylene group, a substituted or unsubstituted m-phenylene group, or a substituted or unsubstituted p-phenylene group.

In an exemplary arrangement of the organic EL device of the exemplary embodiment, L₃₃ is a substituted or unsubstituted o-phenylene group or a substituted or unsubstituted p-phenylene group.

In an exemplary arrangement of the organic EL device of the exemplary embodiment, also preferably, L₃₁ to L₃₃ are each independently a single bond or a group represented by a formula (L₁) below.

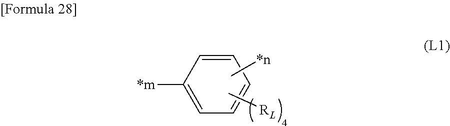

In the formula (L₁):
R_{L} is a hydrogen atom, a substituted or unsubstituted alkyl group having 1 to 6 carbon atoms, or a substituted or unsubstituted aryl group having 6 to 12 ring carbon atoms;
a plurality of R_{L} are mutually the same or different; and
*m and *n each represent a bonding position.

One of *m and *n represents a bonding position to the central nitrogen atom N*, and the other of *m and *n represents a bonding position to Ar₃₁, Ar₃₂, *e, *p, *m, or *n.

In an exemplary arrangement of the organic EL device of the exemplary embodiment, L₃₃ is also preferably a group represented by the formula (L₁).

In an exemplary arrangement of the organic EL device of the exemplary embodiment, the compound represented by the formula (EB1) is a compound represented by a formula (EB101) below.

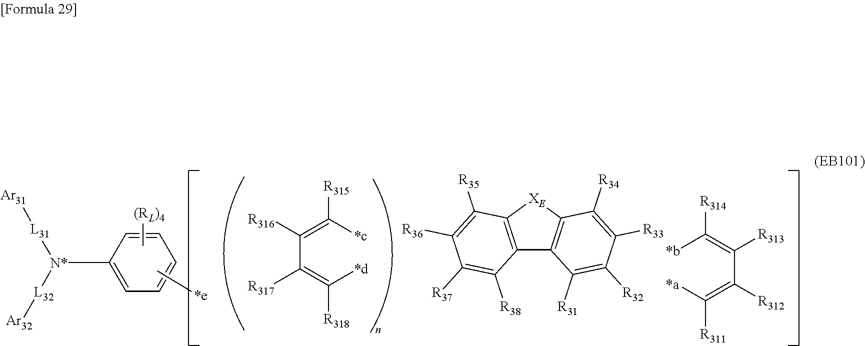

In the formula (EB101), N*, R₃₁ to R₃₈, R₃₁₁ to R₃₁₈, X_{E}, Ar₃₁, Ar₃₂, L₃₁, L₃₂, *a, *b, *c, *d, *e, and n respectively represent the same as those defined in the formula (EB1), and R_{L} represents the same as that defined in the formula (L₁).

In an exemplary arrangement of the organic EL device of the exemplary embodiment, the compound represented by the formula (EB1) is a compound represented by a formula (EB111), (EB112), or (EB113) below.

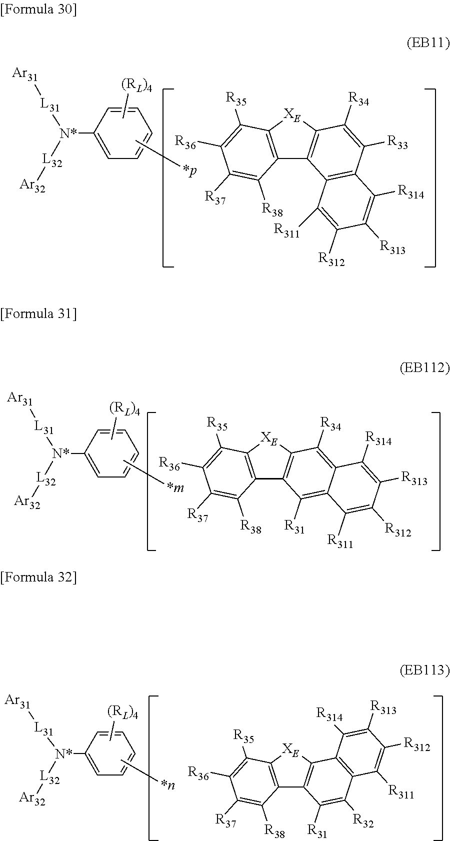

In the formulae (EB111), (EB112), and (EB113):
N*, R₃₁ to R₃₈, R₃₁₁ to R₃₁₄, X_{E}, Ar₃₁, Ar₃₂, L₃₁, and L₃₂ respectively represent the same as those defined in the formula (EB1);
R_{L} represents the same as that defined in the formula (L₁);
when the compound represented by the formula (EB1) is a compound represented by the formula (EB111), one selected from R₃₃ to R₃₈ and R₃₁₁ to R₃₁₄ is a single bond with *p;
when the compound represented by the formula (EB1) is a compound represented by the formula (EB112), one selected from R₃₁, R₃₄ to R₃₈, and R₃₁₁ to R₃₁₄ is a single bond with *m; and
when the compound represented by the formula (EB1) is a compound represented by the formula (EB113), one selected from R₃₁, R₃₂, R₃₅ to R₃₈, and R₃₁₁ to R₃₁₄ is a single bond with *n.

In an exemplary arrangement of the organic EL device of the exemplary embodiment, R₃₁, R₃₅, R₃₇, R₃₈, or R₃₁₁ in the formula (EB111), (EB112), or (EB113) is a single bond with *p, *m, or *n.

In an exemplary arrangement of the organic EL device of the exemplary embodiment, R₃₈ in the formula (EB111), (EB112), or (EB113) is a single bond with *p, *m, or *n.

In an exemplary arrangement of the organic EL device of the exemplary embodiment, when R₃₈ in the formula (EB113) is a single bond with *n, the compound represented by the formula (EB113) is a compound represented by a formula (EB114) below.

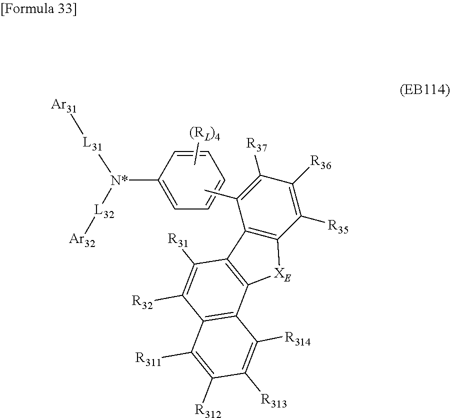

In the formula (EB114), N*, R₃₁, R₃₂, R₃₅ to R₃₇, R₃₁₁ to R₃₁₄, X_{E}, Ar₃₁, Ar₃₂, L₃₁, and L₃₂ respectively represent the same as those defined in the formula (EB1), and R_{L} represents the same as that defined in the formula (L₁).

In an exemplary arrangement of the organic EL device of the exemplary embodiment, the compound represented by the formula (EB1) is a compound represented by a formula (EB132) below.

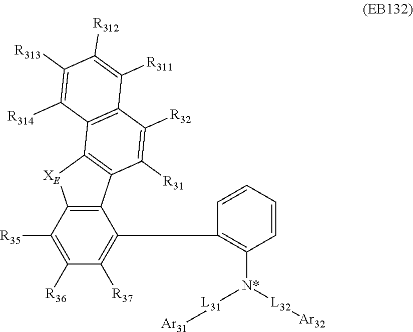

In the formula (EB132), N*, R₃₁, R₃₂, R₃₅ to R₃₇, R₃₁₁ to R₃₁₄, X_{E}, Ar₃₁, Ar₃₂, L₃₁, and L₃₂ respectively represent the same as those defined in the formula (EB1).

In an exemplary arrangement of the organic EL device of the exemplary embodiment, X_{E} is an oxygen atom.

In an exemplary arrangement of the organic EL device of the exemplary embodiment, Ar₃₁ and Ar₃₂ are each independently a group represented by any of formulae (1-a) to (1-f) below.

In an exemplary arrangement of the organic EL device of the exemplary embodiment, Ar₃₁ and Ar₃₂ in the formula (EB1) are each independently a group represented by any of the formulae (1-a) to (1-f);
when Ar₃₁ is a group represented by any of the formulae (1-a) to (1-f), L₃₁ is a single bond or an unsubstituted arylene group having 6 to 30 ring carbon atoms; and
when Ar₃₂ is a group represented by any of the formulae (1-a) to (1-f), L₃₂ is a single bond or an unsubstituted arylene group having 6 to 30 ring carbon atoms.

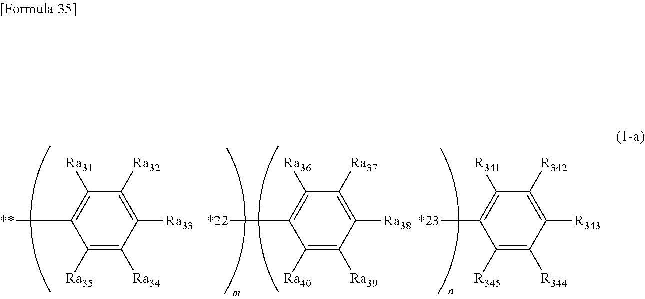

In the formula (1-a):
R₃₄₁ to R₃₄₅ are each independently a hydrogen atom, a substituted or unsubstituted alkyl group having 1 to 10 carbon atoms, or a substituted or unsubstituted aryl group having 6 to 12 ring carbon atoms;
adjacent two selected from R₃₄₁ to R₃₄₅ are not mutually bonded and thus form no ring;
one selected from Ra₃₁ to Ra₃₅ is a single bond with *22;
one selected from Ra₃₆ to Ra₄₀ is a single bond with *23;
Ra₃₁ to Ra₄₀ not being the single bond are each independently a hydrogen atom, an unsubstituted alkyl group having 1 to 10 carbon atoms, or an unsubstituted aryl group having 6 to 12 ring carbon atoms;
adjacent two selected from Ra₃₁ to Ra₃₅ not being the single bond are not mutually bonded and thus form no ring;
adjacent two selected from Ra₃₆ to Ra₄₀ not being the single bond are not mutually bonded and thus form no ring;
** represents a bonding position to L₃₁ or L₃₂;
m is 0 or 1, and n is 0 or 1;
when m and n are each 0, *23 represents a bonding position to L₃₁ or L₃₂;
when m is 0 and n is 1, *22 represents a bonding position to L₃₁ or L₃₂;
when m is 1 and n is 0, one selected from Ra₃₁ to Ra₃₅ is a single bond with *23;
when L₃₁ is a single bond, **, *22, or *23 of the group represented by the formula (1-a), which is Ar₃₁, represents a bonding position to the central nitrogen atom N*; and
when L₃₂ is a single bond, **, *22, or *23 of the group represented by the formula (1-a), which is Ar₃₂, represents a bonding position to the central nitrogen atom N*.

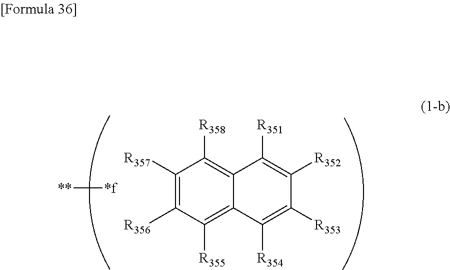

In the formula (1-b):
R₃₅₁ to R₃₅₈ are each independently a hydrogen atom, a substituted or unsubstituted alkyl group having 1 to 6 carbon atoms, or a substituted or unsubstituted aryl group having 6 to 12 ring carbon atoms;
one selected from R₃₅₁ to R₃₅₈ is a single bond with *f;
adjacent two selected from R₃₅₁ to R₃₅₈ not being the single bond are not mutually bonded and thus form no cyclic structure;
** represents a bonding position to L₃₁ or L₃₂;
when L₃₁ is a single bond, ** of the group represented by the formula (1-b), which is Ar₃₁, represents a bonding position to the central nitrogen atom N*; and
when L₃₂ is a single bond, ** of the group represented by the formula (1-b), which is Ar₃₂, represents a bonding position to the central nitrogen atom N*.

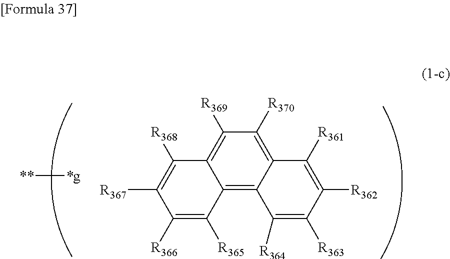

In the formula (1-c):
R₃₆₁ to R₃₇₀ are each independently a hydrogen atom, a substituted or unsubstituted alkyl group having 1 to 6 carbon atoms, or a substituted or unsubstituted aryl group having 6 to 12 ring carbon atoms;
one selected from R₃₆₁ to R₃₇₀ is a single bond with *g;
adjacent two selected from R₃₆₁ to R₃₇₀ not being the single bond are not mutually bonded and thus form no cyclic structure;
** represents a bonding position to L₃₁ or L₃₂;
when L₃₁ is a single bond, ** of the group represented by the formula (1-c), which is Ar₃₁, represents a bonding position to the central nitrogen atom N*; and
when L₃₂ is a single bond, ** of the group represented by the formula (1-c), which is Ar₃₂, represents a bonding position to the central nitrogen atom N*.

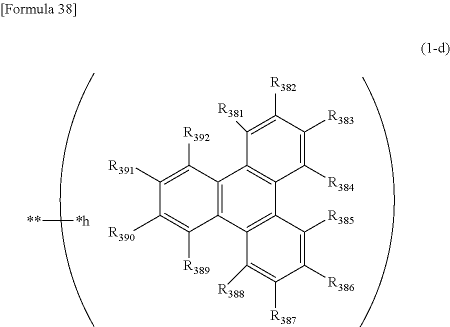

In the formula (1-d):
R₃₈₁ to R₃₉₂ are each independently a hydrogen atom, a substituted or unsubstituted alkyl group having 1 to 6 carbon atoms, or a substituted or unsubstituted aryl group having 6 to 12 ring carbon atoms;
one selected from R₃₈₁ to R₃₉₂ is a single bond with *h;
adjacent two selected from R₃₈₁ to R₃₉₂ not being the single bond are not mutually bonded and thus form no cyclic structure;
** represents a bonding position to L₃₁ or L₃₂;
when L₃₁ is a single bond, ** of the group represented by the formula (1-d), which is Ar₃₁, represents a bonding position to the central nitrogen atom N*; and
when L₃₂ is a single bond, ** of the group represented by the formula (1-d), which is Ar₃₂, represents a bonding position to the central nitrogen atom N*.

[Formula 39]

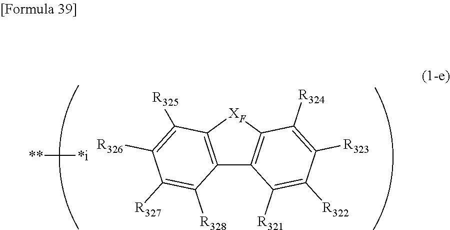

In the formula (1-e):
R₃₂₁ to R₃₂₈ are each independently a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
X_{F} is an oxygen atom, a sulfur atom, NR_{F1}, or CR_{F2}R_{F3};
R_{F1} is a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
R_{F2} and R_{F3} are each independently a hydrogen atom, a substituted or unsubstituted alkyl group having 1 to 50 carbon atoms, or a substituted or unsubstituted aryl group having 6 to 50 ring carbon atoms, and R_{F2} and R_{F3} are mutually bonded to form a substituted or unsubstituted monocyclic ring, mutually bonded to form a substituted or unsubstituted fused ring, or not mutually bonded;
one selected from R₃₂₁ to R₃₂₈, R_{F1}, R_{F2}, and R_{F3} is a single bond with *i; adjacent two selected from R₃₂₁ to R₃₂₈ not being the single bond are mutually bonded to form a substituted or unsubstituted benzene ring, or not mutually bonded;
** represents a bonding position to L₃₁ or L₃₂;
when L₃₁ is a single bond, ** of the group represented by the formula (1-e), which is Ar₃₁, represents a bonding position to the central nitrogen atom N*; and
when L₃₂ is a single bond, ** of the group represented by the formula (1-e), which is Ar₃₂, represents a bonding position to the central nitrogen atom N*.

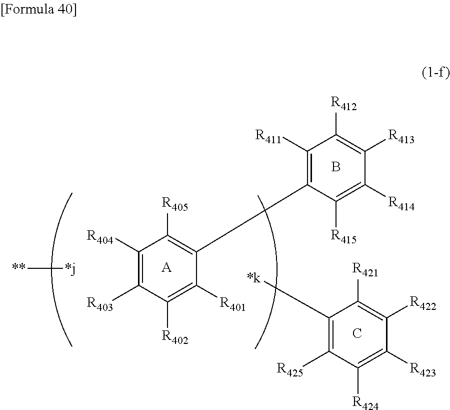

In the formula (1-f):
R₄₀₁ to R₄₀₅ are each independently a hydrogen atom, an unsubstituted alkyl group having 1 to 6 carbon atoms, or an unsubstituted phenyl group;
R₄₁₁ to R₄₁₅ and R₄₂₁ to R₄₂₅ are each independently a hydrogen atom or an unsubstituted alkyl group having 1 to 6 carbon atoms;
one selected from R₄₀₁ to R₄₀₅ is a single bond with *j;
another one selected from R₄₀₁ to R₄₀₅ is a single bond with *k;
adjacent two selected from R₄₀₁ to R₄₀₅ not being the single bond are not mutually bonded and thus form no cyclic structure;
adjacent two selected from R₄₁₁ to R₄₁₅ and R₄₂₁ to R₄₂₅ are mutually bonded to form a substituted or unsubstituted benzene ring, or not mutually bonded;
** represents a bonding position to L₃₁ or L₃₂;
when L₃₁ is a single bond, ** of the group represented by the formula (1-f), which is Ar₃₁, represents a bonding position to the central nitrogen atom N*; and
when L₃₂ is a single bond, ** of the group represented by the formula (1-f), which is Ar₃₂, represents a bonding position to the central nitrogen atom N*.

In an exemplary arrangement of the organic EL device of the exemplary embodiment, L₃₁ and L₃₂ are each independently a single bond or a substituted or unsubstituted phenylene group.

In an exemplary arrangement of the organic EL device of the exemplary embodiment, L₃₁ and L₃₂ are each independently a single bond or an unsubstituted phenylene group.

In an exemplary arrangement of the organic EL device of the exemplary embodiment, the third compound is a compound represented by any of the formulae (EB11) to (EB13), the first host material is a compound having, in a molecule, at least one group selected from the group consisting of groups represented by formulae (HX11), (HX12), and (HX13) below, and the second host material is a compound having, in a molecule, at least one group selected from the group consisting of groups represented by formulae (HY11), (HY12), and (HY13) below.

In an exemplary arrangement of the organic EL device of the exemplary embodiment, the third compound is a compound represented by the formula (EB131) or the formula (EB132), the first host material is a compound having, in a molecule, at least one group selected from the group consisting of groups represented by the formulae (HX11), (HX12), and (HX13) below, and the second host material is a compound having, in a molecule, at least one group selected from the group consisting of groups represented by the formulae (HY11), (HY12), and (HY13) below.

In an exemplary arrangement of the organic EL device of the exemplary embodiment, at least one hydrogen atom contained in the third compound is a deuterium atom.

In an exemplary arrangement of the organic EL device of the exemplary embodiment, at least one of hydrogen atoms for L₃₁ to L₃₃ of the third compound is a deuterium atom.

In an exemplary arrangement of the organic EL device of the exemplary embodiment, at least one of hydrogen atoms for L₃₁ and L₃₂ of the third compound is a deuterium atom.

In the third compound, also preferably, the groups specified to be “substituted or unsubstituted” are each an “unsubstituted” group.

### Method of Producing Third Compound

The third compound can be produced by a known method. Further, the third compound can be produced based on a known method through a known alternative reaction using a known material(s) tailored for the target compound.

### Specific Examples of Third Compound

Specific examples of the third compound include the following compounds. The invention, however, is not limited to the specific examples of the third compound.

In the specific examples of the compounds herein, D represents a deuterium atom, Me represents a methyl group, tBu represents a tert-butyl group, and Ph represents a phenyl group.

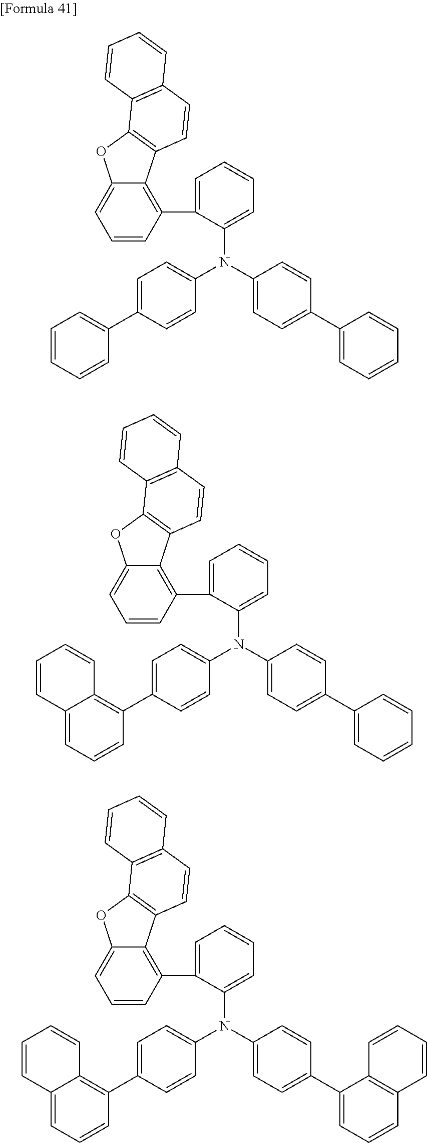 [SMILES: c1ccc(-c2ccc(N(c3ccc(-c4ccccc4)cc3)c3ccccc3-c3cccc4oc5c6ccccc6ccc5c34)cc2)cc1 ; c1ccc(-c2ccc(N(c3ccc(-c4cccc5ccccc45)cc3)c3ccccc3-c3cccc4oc5c6ccccc6ccc5c34)cc2)cc1 ; c1ccc(N(c2ccc(-c3cccc4ccccc34)cc2)c2ccc(-c3cccc4ccccc34)cc2)c(-c2cccc3oc4c5ccccc5ccc4c23)c1]

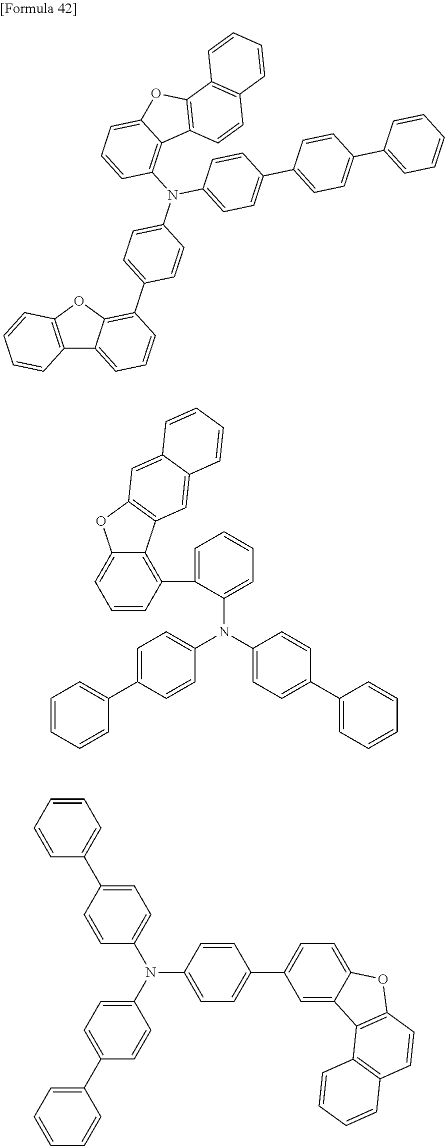 [SMILES: c1ccc(-c2ccc(-c3ccc(N(c4ccc(-c5cccc6c5oc5ccccc56)cc4)c4cccc5oc6c7ccccc7ccc6c45)cc3)cc2)cc1 ; c1ccc(-c2ccc(N(c3ccc(-c4ccccc4)cc3)c3ccccc3-c3cccc4oc5cc6ccccc6cc5c34)cc2)cc1 ; c1ccc(-c2ccc(N(c3ccc(-c4ccccc4)cc3)c3ccc(-c4ccc5oc6ccc7ccccc7c6c5c4)cc3)cc2)cc1]

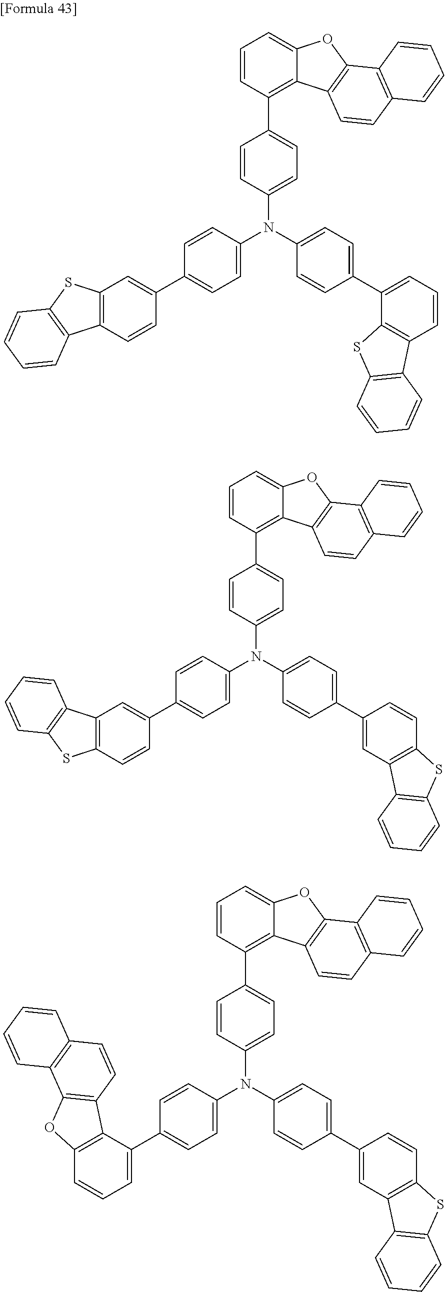 [SMILES: c1ccc2c(c1)ccc1c2oc2cccc(-c3ccc(N(c4ccc(-c5ccc6c(c5)sc5ccccc56)cc4)c4ccc(-c5cccc6c5sc5ccccc56)cc4)cc3)c21 ; c1ccc2c(c1)ccc1c2oc2cccc(-c3ccc(N(c4ccc(-c5ccc6sc7ccccc7c6c5)cc4)c4ccc(-c5ccc6sc7ccccc7c6c5)cc4)cc3)c21 ; c1ccc2c(c1)ccc1c2oc2cccc(-c3ccc(N(c4ccc(-c5ccc6sc7ccccc7c6c5)cc4)c4ccc(-c5cccc6oc7c8ccccc8ccc7c56)cc4)cc3)c21]

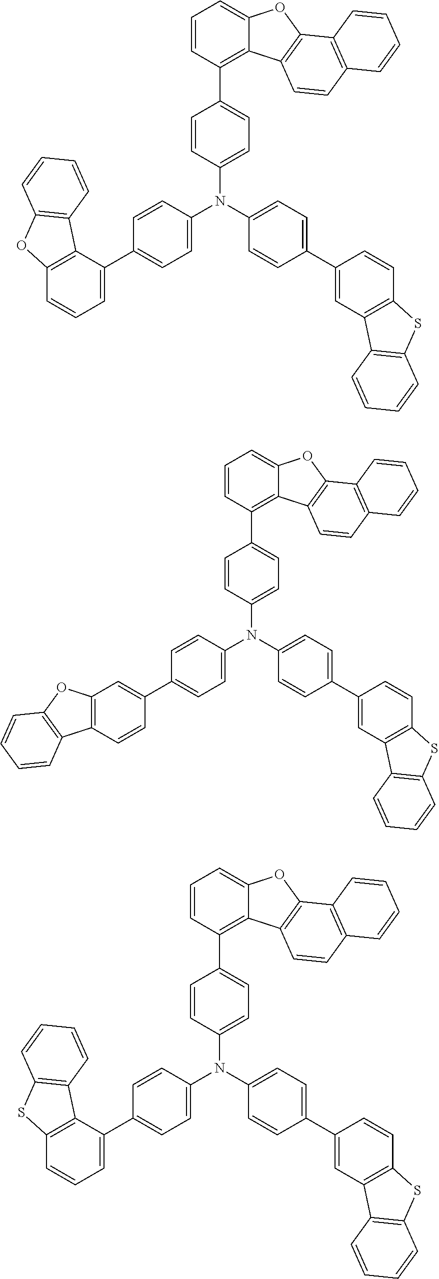 [SMILES: c1ccc2c(c1)ccc1c2oc2cccc(-c3ccc(N(c4ccc(-c5ccc6sc7ccccc7c6c5)cc4)c4ccc(-c5cccc6oc7ccccc7c56)cc4)cc3)c21 ; c1ccc2c(c1)ccc1c2oc2cccc(-c3ccc(N(c4ccc(-c5ccc6c(c5)oc5ccccc56)cc4)c4ccc(-c5ccc6sc7ccccc7c6c5)cc4)cc3)c21 ; c1ccc2c(c1)ccc1c2oc2cccc(-c3ccc(N(c4ccc(-c5ccc6sc7ccccc7c6c5)cc4)c4ccc(-c5cccc6sc7ccccc7c56)cc4)cc3)c21]

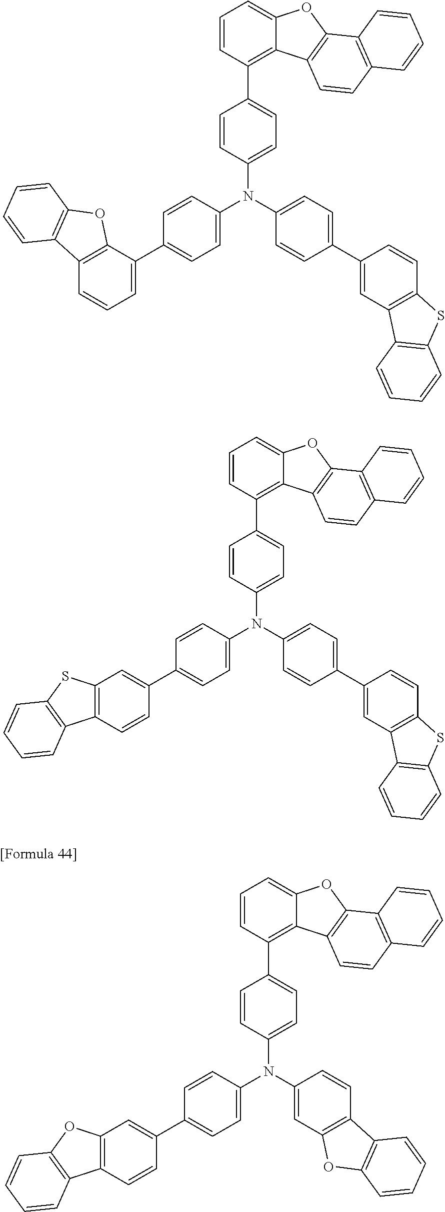 [SMILES: c1ccc2c(c1)ccc1c2oc2cccc(-c3ccc(N(c4ccc(-c5ccc6sc7ccccc7c6c5)cc4)c4ccc(-c5cccc6c5oc5ccccc56)cc4)cc3)c21 ; c1ccc2c(c1)ccc1c2oc2cccc(-c3ccc(N(c4ccc(-c5ccc6c(c5)sc5ccccc56)cc4)c4ccc(-c5ccc6sc7ccccc7c6c5)cc4)cc3)c21 ; c1ccc2c(c1)ccc1c2oc2cccc(-c3ccc(N(c4ccc(-c5ccc6c(c5)oc5ccccc56)cc4)c4ccc5c(c4)oc4ccccc45)cc3)c21]

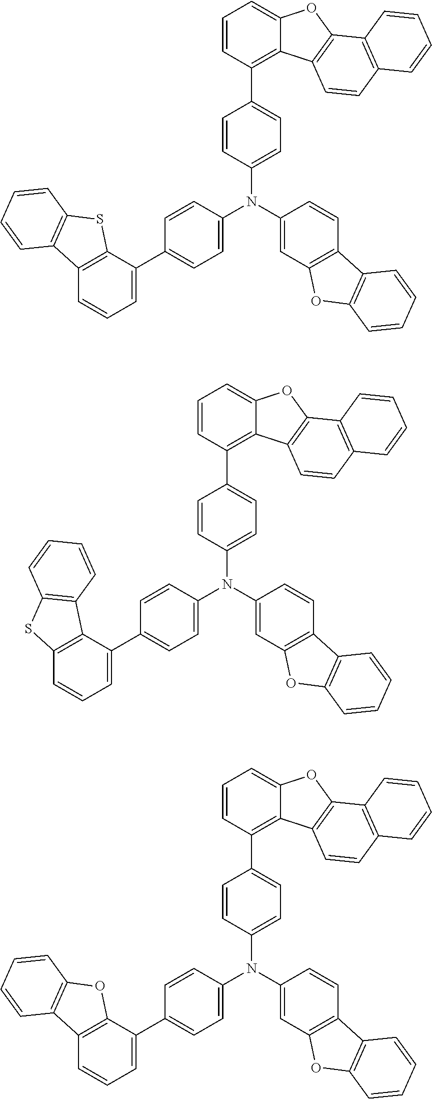 [SMILES: c1ccc2c(c1)ccc1c2oc2cccc(-c3ccc(N(c4ccc(-c5cccc6c5sc5ccccc56)cc4)c4ccc5c(c4)oc4ccccc45)cc3)c21 ; c1ccc2c(c1)ccc1c2oc2cccc(-c3ccc(N(c4ccc(-c5cccc6sc7ccccc7c56)cc4)c4ccc5c(c4)oc4ccccc45)cc3)c21 ; c1ccc2c(c1)ccc1c2oc2cccc(-c3ccc(N(c4ccc(-c5cccc6c5oc5ccccc56)cc4)c4ccc5c(c4)oc4ccccc45)cc3)c21]

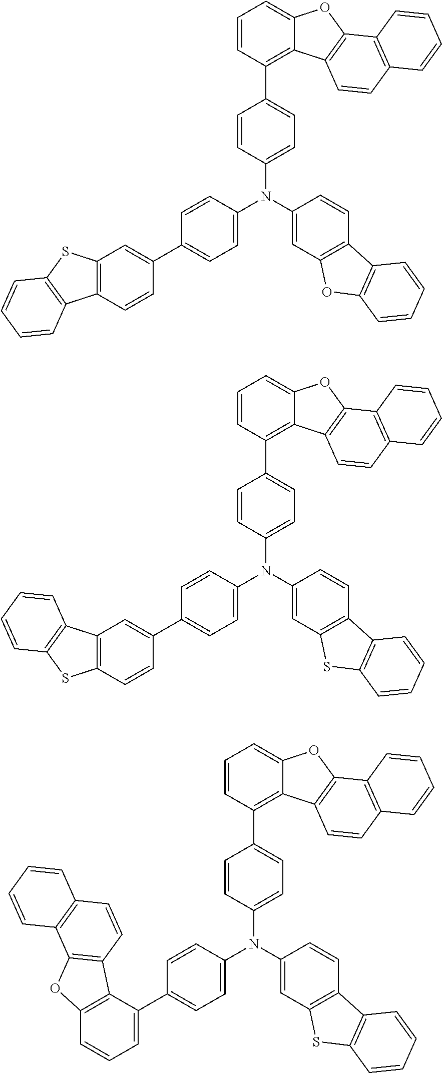 [SMILES: c1ccc2c(c1)ccc1c2oc2cccc(-c3ccc(N(c4ccc(-c5ccc6c(c5)sc5ccccc56)cc4)c4ccc5c(c4)oc4ccccc45)cc3)c21 ; c1ccc2c(c1)ccc1c2oc2cccc(-c3ccc(N(c4ccc(-c5ccc6sc7ccccc7c6c5)cc4)c4ccc5c(c4)sc4ccccc45)cc3)c21 ; c1ccc2c(c1)ccc1c2oc2cccc(-c3ccc(N(c4ccc(-c5cccc6oc7c8ccccc8ccc7c56)cc4)c4ccc5c(c4)sc4ccccc45)cc3)c21]

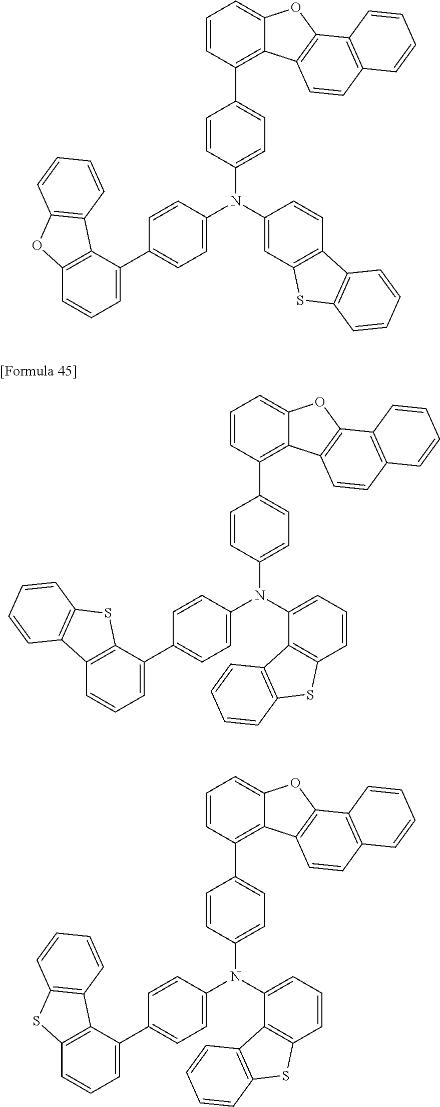 [SMILES: c1ccc2c(c1)ccc1c2oc2cccc(-c3ccc(N(c4ccc(-c5cccc6oc7ccccc7c56)cc4)c4ccc5c(c4)sc4ccccc45)cc3)c21 ; c1ccc2c(c1)ccc1c2oc2cccc(-c3ccc(N(c4ccc(-c5cccc6c5sc5ccccc56)cc4)c4cccc5sc6ccccc6c45)cc3)c21 ; c1ccc2c(c1)ccc1c2oc2cccc(-c3ccc(N(c4ccc(-c5cccc6sc7ccccc7c56)cc4)c4cccc5sc6ccccc6c45)cc3)c21]

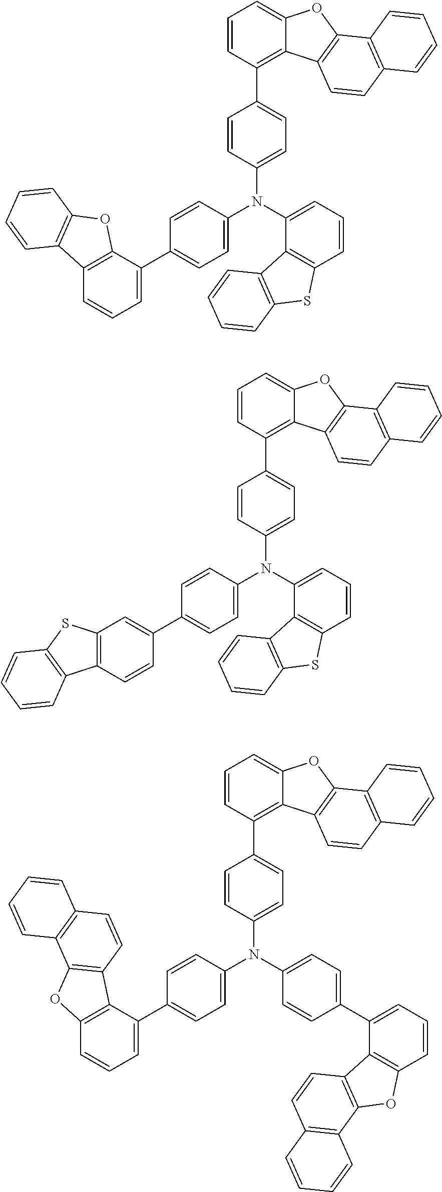

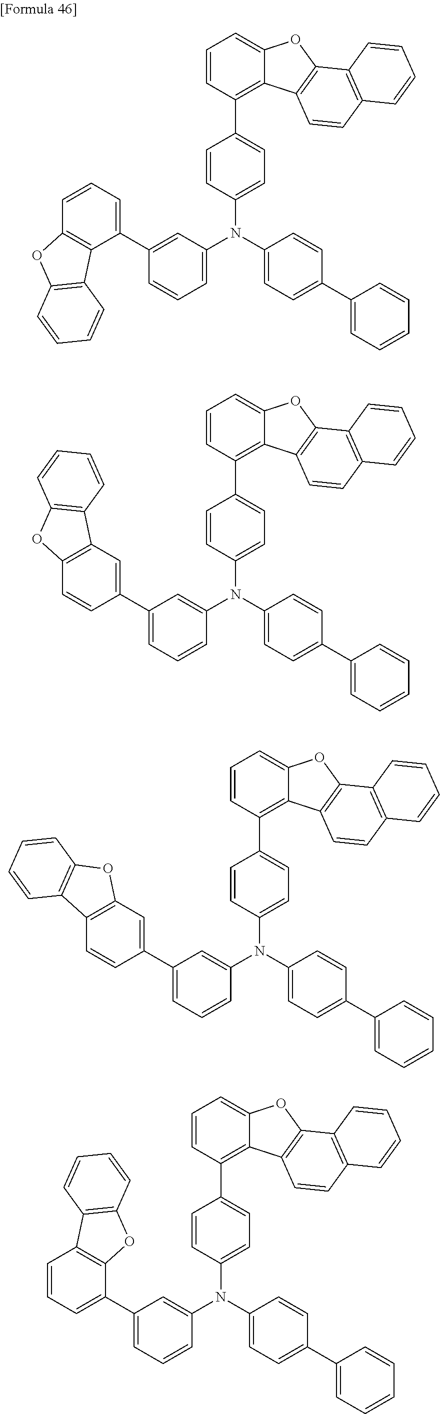

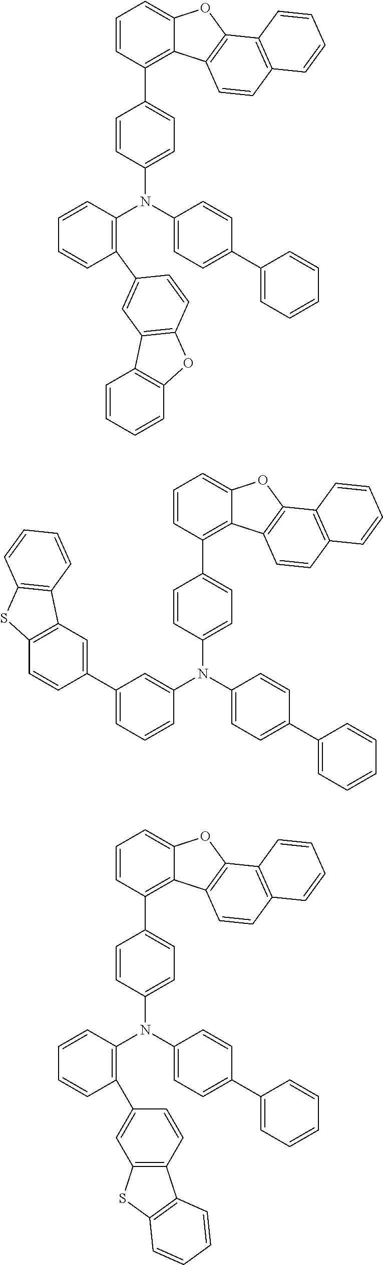 [SMILES: c1ccc(-c2ccc(N(c3ccc(-c4cccc5oc6c7ccccc7ccc6c45)cc3)c3ccccc3-c3ccc4oc5ccccc5c4c3)cc2)cc1 ; c1ccc(-c2ccc(N(c3ccc(-c4cccc5oc6c7ccccc7ccc6c45)cc3)c3cccc(-c4ccc5sc6ccccc6c5c4)c3)cc2)cc1 ; c1ccc(-c2ccc(N(c3ccc(-c4cccc5oc6c7ccccc7ccc6c45)cc3)c3ccccc3-c3ccc4c(c3)sc3ccccc34)cc2)cc1]

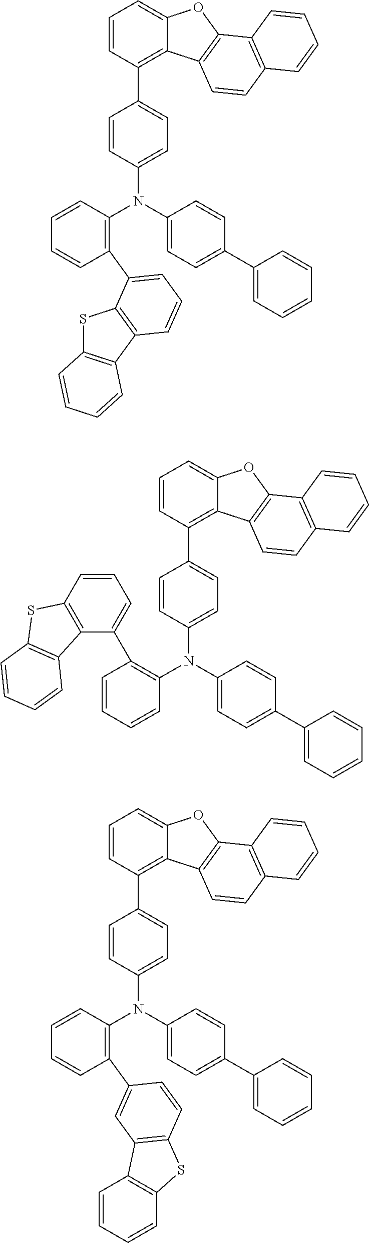 [SMILES: c1ccc(-c2ccc(N(c3ccc(-c4cccc5oc6c7ccccc7ccc6c45)cc3)c3ccccc3-c3cccc4c3sc3ccccc34)cc2)cc1 ; c1ccc(-c2ccc(N(c3ccc(-c4cccc5oc6c7ccccc7ccc6c45)cc3)c3ccccc3-c3cccc4sc5ccccc5c34)cc2)cc1 ; c1ccc(-c2ccc(N(c3ccc(-c4cccc5oc6c7ccccc7ccc6c45)cc3)c3ccccc3-c3ccc4sc5ccccc5c4c3)cc2)cc1]

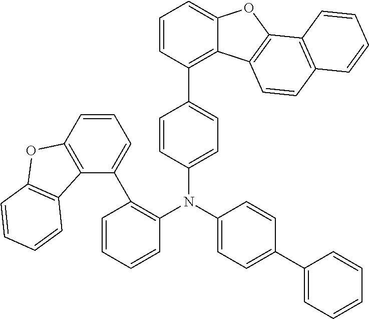 [SMILES: c1ccc(-c2ccc(N(c3ccc(-c4cccc5oc6c7ccccc7ccc6c45)cc3)c3ccccc3-c3cccc4oc5ccccc5c34)cc2)cc1]

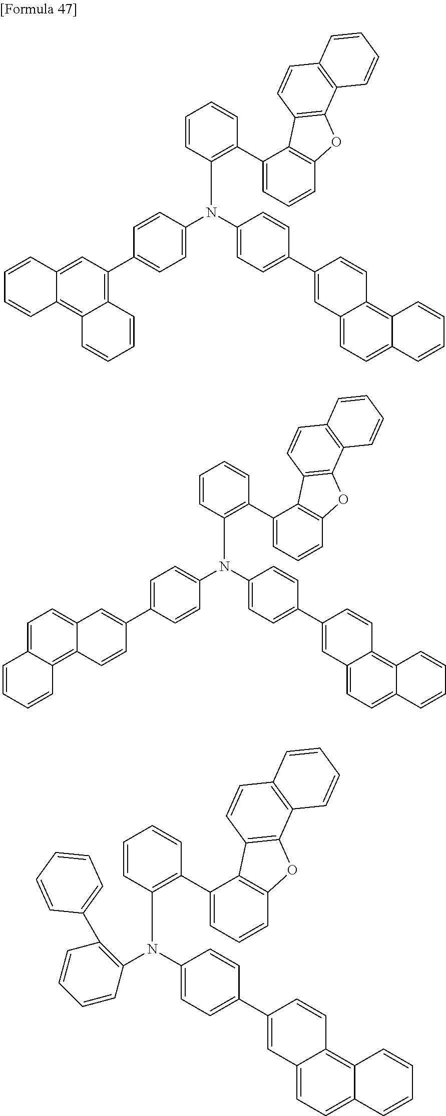

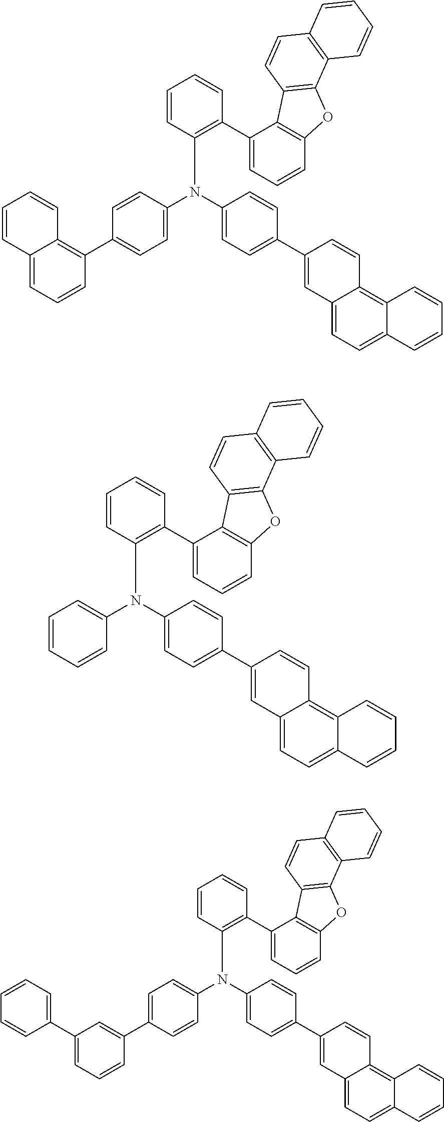

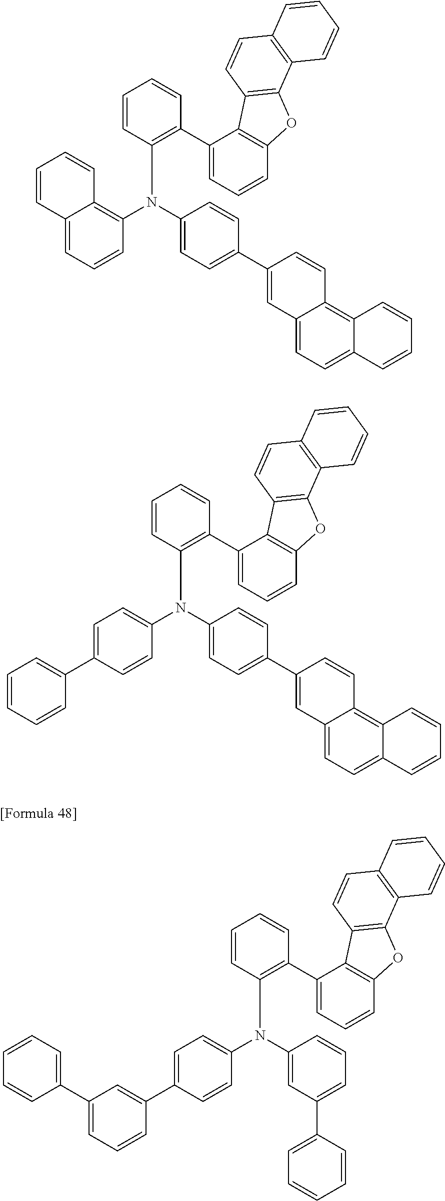

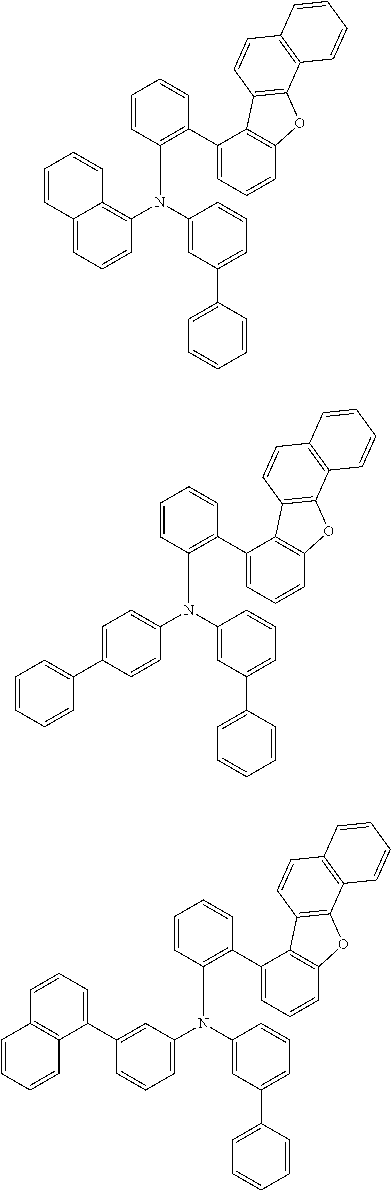 [SMILES: c1ccc(-c2cccc(N(c3ccccc3-c3cccc4oc5c6ccccc6ccc5c34)c3cccc4ccccc34)c2)cc1 ; c1ccc(-c2ccc(N(c3cccc(-c4ccccc4)c3)c3ccccc3-c3cccc4oc5c6ccccc6ccc5c34)cc2)cc1 ; c1ccc(-c2cccc(N(c3cccc(-c4cccc5ccccc45)c3)c3ccccc3-c3cccc4oc5c6ccccc6ccc5c34)c2)cc1]

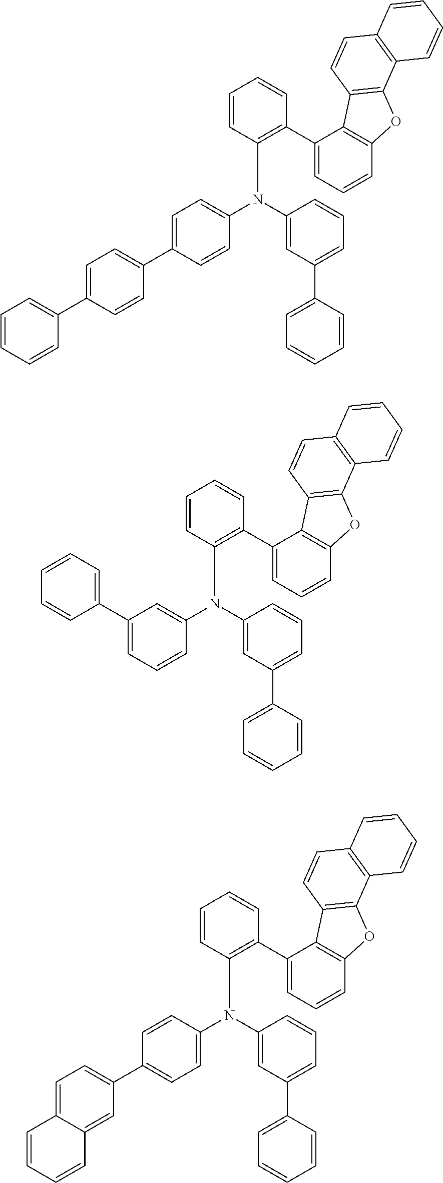 [SMILES: c1ccc(-c2ccc(-c3ccc(N(c4cccc(-c5ccccc5)c4)c4ccccc4-c4cccc5oc6c7ccccc7ccc6c45)cc3)cc2)cc1 ; c1ccc(-c2cccc(N(c3cccc(-c4ccccc4)c3)c3ccccc3-c3cccc4oc5c6ccccc6ccc5c34)c2)cc1 ; c1ccc(-c2cccc(N(c3ccc(-c4ccc5ccccc5c4)cc3)c3ccccc3-c3cccc4oc5c6ccccc6ccc5c34)c2)cc1]

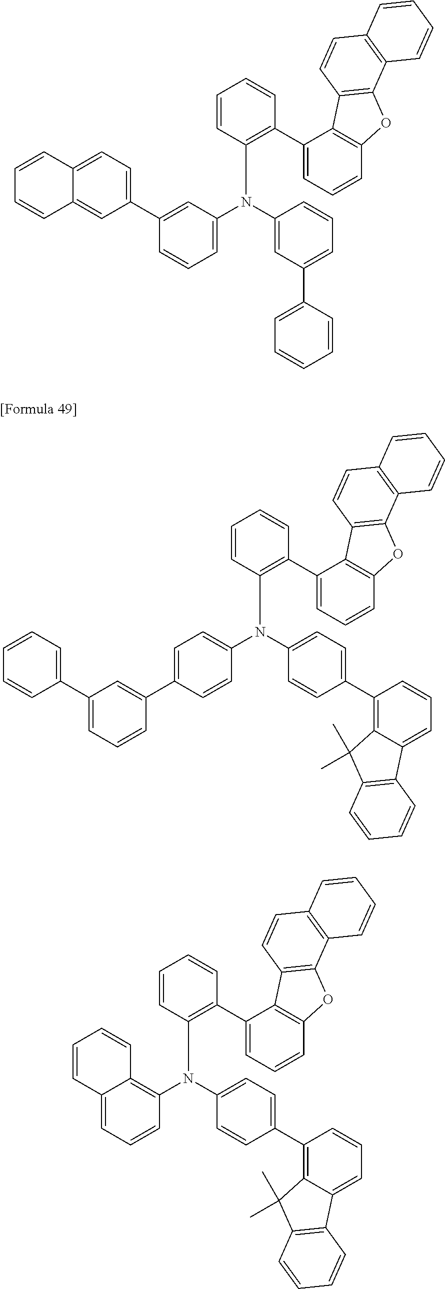

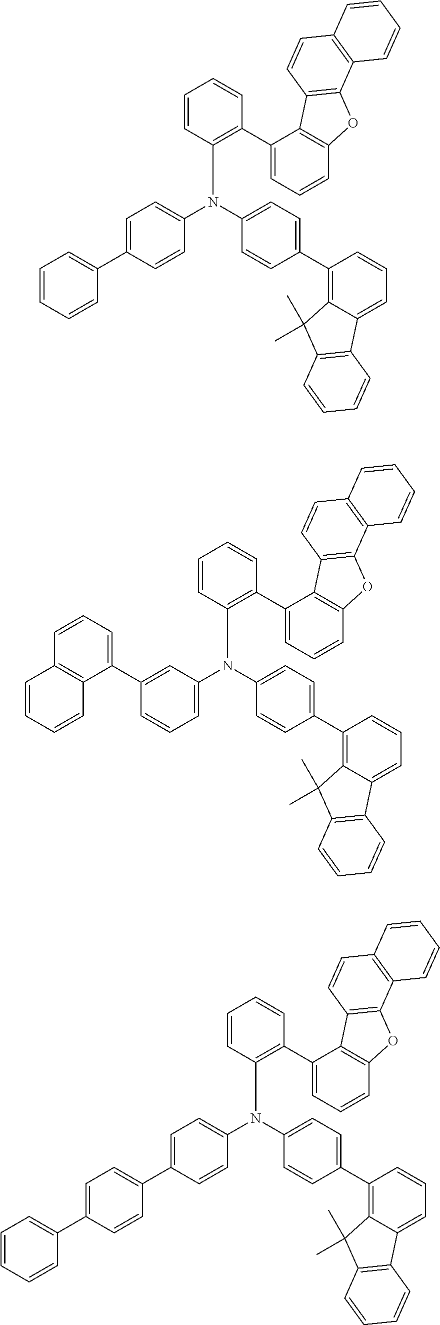 [SMILES: CC1(C)c2ccccc2-c2cccc(-c3ccc(N(c4ccc(-c5ccccc5)cc4)c4ccccc4-c4cccc5oc6c7ccccc7ccc6c45)cc3)c21 ; CC1(C)c2ccccc2-c2cccc(-c3ccc(N(c4cccc(-c5cccc6ccccc56)c4)c4ccccc4-c4cccc5oc6c7ccccc7ccc6c45)cc3)c21 ; CC1(C)c2ccccc2-c2cccc(-c3ccc(N(c4ccc(-c5ccc(-c6ccccc6)cc5)cc4)c4ccccc4-c4cccc5oc6c7ccccc7ccc6c45)cc3)c21]

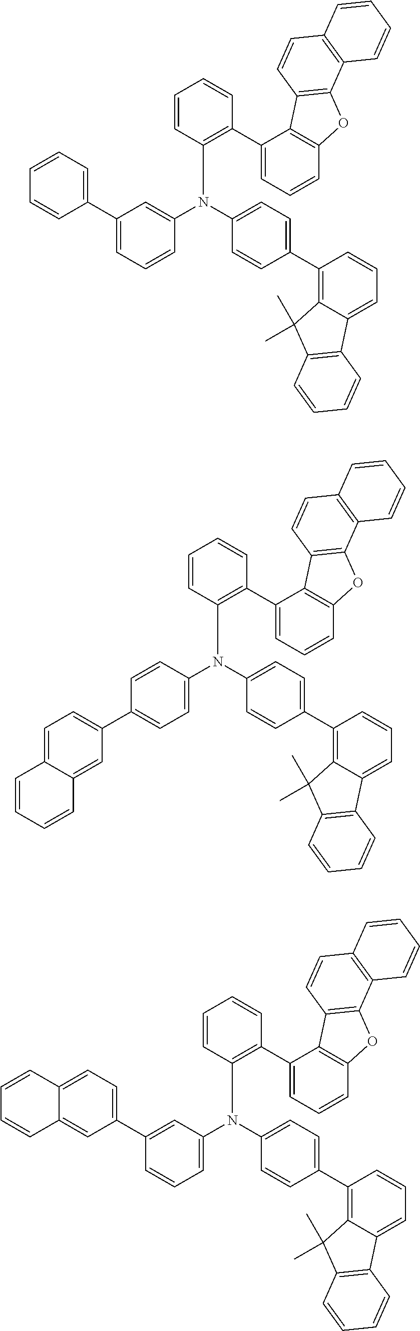 [SMILES: CC1(C)c2ccccc2-c2cccc(-c3ccc(N(c4cccc(-c5ccccc5)c4)c4ccccc4-c4cccc5oc6c7ccccc7ccc6c45)cc3)c21 ; CC1(C)c2ccccc2-c2cccc(-c3ccc(N(c4ccc(-c5ccc6ccccc6c5)cc4)c4ccccc4-c4cccc5oc6c7ccccc7ccc6c45)cc3)c21 ; CC1(C)c2ccccc2-c2cccc(-c3ccc(N(c4cccc(-c5ccc6ccccc6c5)c4)c4ccccc4-c4cccc5oc6c7ccccc7ccc6c45)cc3)c21]

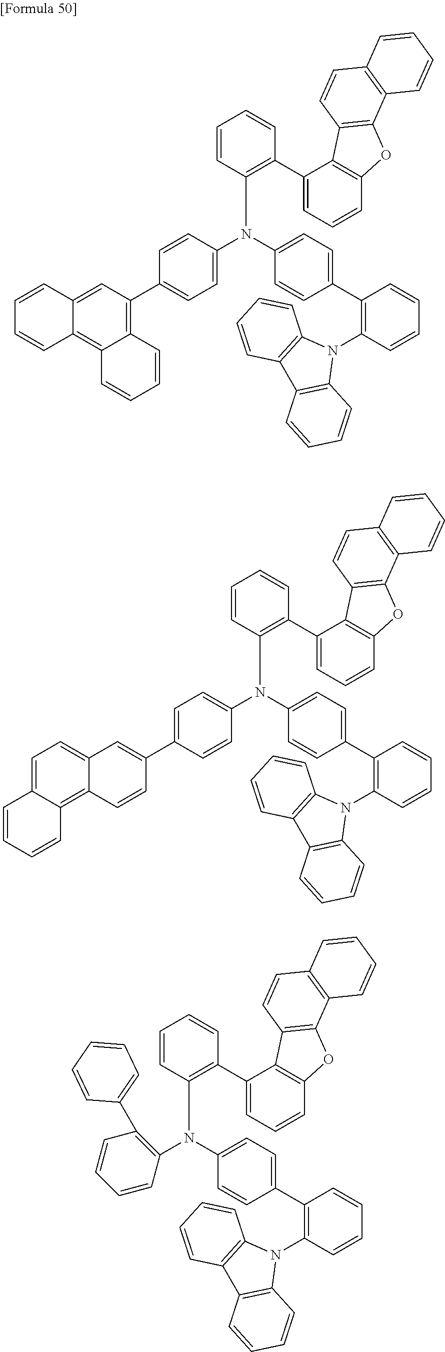

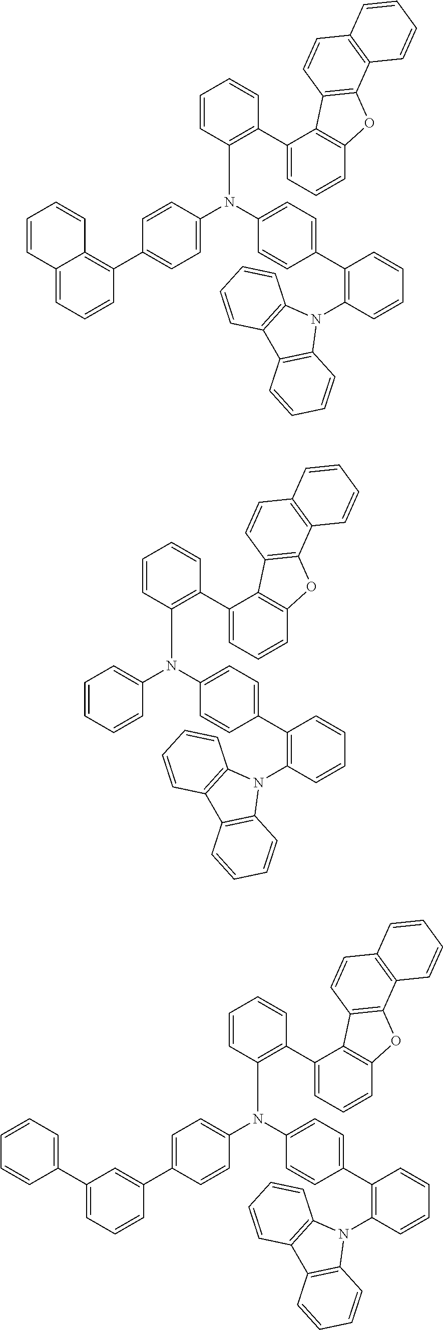 [SMILES: c1ccc(N(c2ccc(-c3ccccc3-n3c4ccccc4c4ccccc43)cc2)c2ccc(-c3cccc4ccccc34)cc2)c(-c2cccc3oc4c5ccccc5ccc4c23)c1 ; c1ccc(N(c2ccc(-c3ccccc3-n3c4ccccc4c4ccccc43)cc2)c2ccccc2-c2cccc3oc4c5ccccc5ccc4c23)cc1 ; c1ccc(-c2cccc(-c3ccc(N(c4ccc(-c5ccccc5-n5c6ccccc6c6ccccc65)cc4)c4ccccc4-c4cccc5oc6c7ccccc7ccc6c45)cc3)c2)cc1]

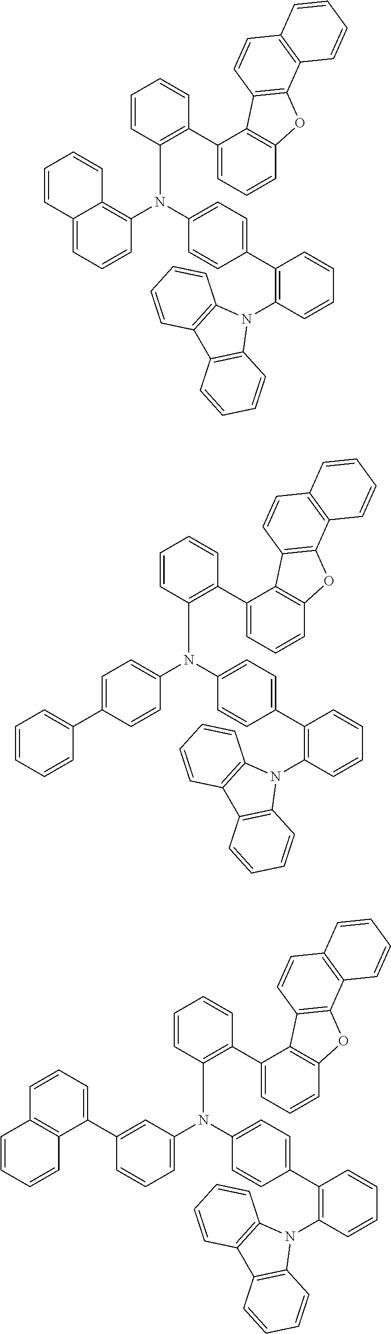 [SMILES: c1ccc(N(c2ccc(-c3ccccc3-n3c4ccccc4c4ccccc43)cc2)c2cccc3ccccc23)c(-c2cccc3oc4c5ccccc5ccc4c23)c1 ; c1ccc(-c2ccc(N(c3ccc(-c4ccccc4-n4c5ccccc5c5ccccc54)cc3)c3ccccc3-c3cccc4oc5c6ccccc6ccc5c34)cc2)cc1 ; c1cc(-c2cccc3ccccc23)cc(N(c2ccc(-c3ccccc3-n3c4ccccc4c4ccccc43)cc2)c2ccccc2-c2cccc3oc4c5ccccc5ccc4c23)c1]

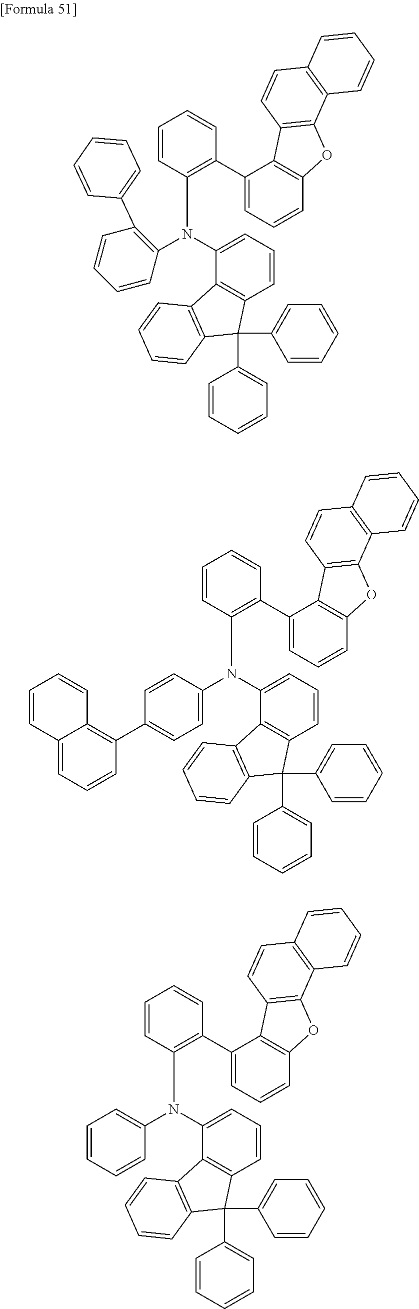

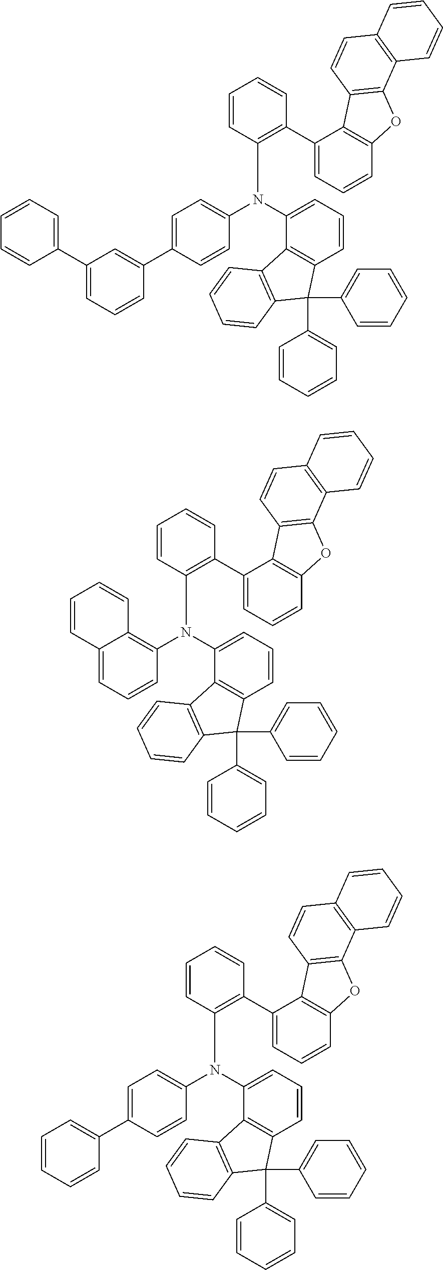

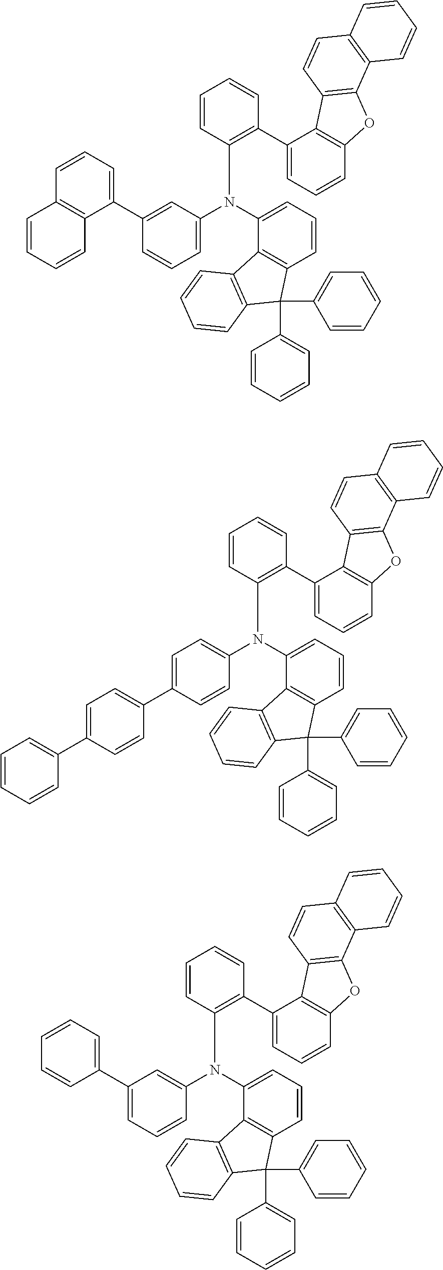 [SMILES: c1ccc(C2(c3ccccc3)c3ccccc3-c3c(N(c4cccc(-c5cccc6ccccc56)c4)c4ccccc4-c4cccc5oc6c7ccccc7ccc6c45)cccc32)cc1 ; c1ccc(-c2ccc(-c3ccc(N(c4ccccc4-c4cccc5oc6c7ccccc7ccc6c45)c4cccc5c4-c4ccccc4C5(c4ccccc4)c4ccccc4)cc3)cc2)cc1 ; c1ccc(-c2cccc(N(c3ccccc3-c3cccc4oc5c6ccccc6ccc5c34)c3cccc4c3-c3ccccc3C4(c3ccccc3)c3ccccc3)c2)cc1]

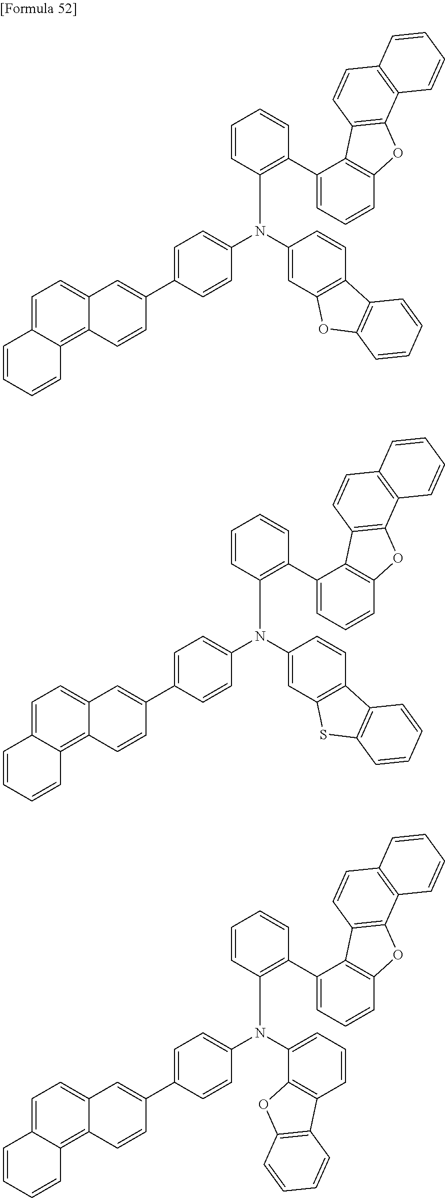 [SMILES: c1ccc(N(c2ccc(-c3ccc4c(ccc5ccccc54)c3)cc2)c2ccc3c(c2)oc2ccccc23)c(-c2cccc3oc4c5ccccc5ccc4c23)c1 ; c1ccc(N(c2ccc(-c3ccc4c(ccc5ccccc54)c3)cc2)c2ccc3c(c2)sc2ccccc23)c(-c2cccc3oc4c5ccccc5ccc4c23)c1 ; c1ccc(N(c2ccc(-c3ccc4c(ccc5ccccc54)c3)cc2)c2cccc3c2oc2ccccc23)c(-c2cccc3oc4c5ccccc5ccc4c23)c1]

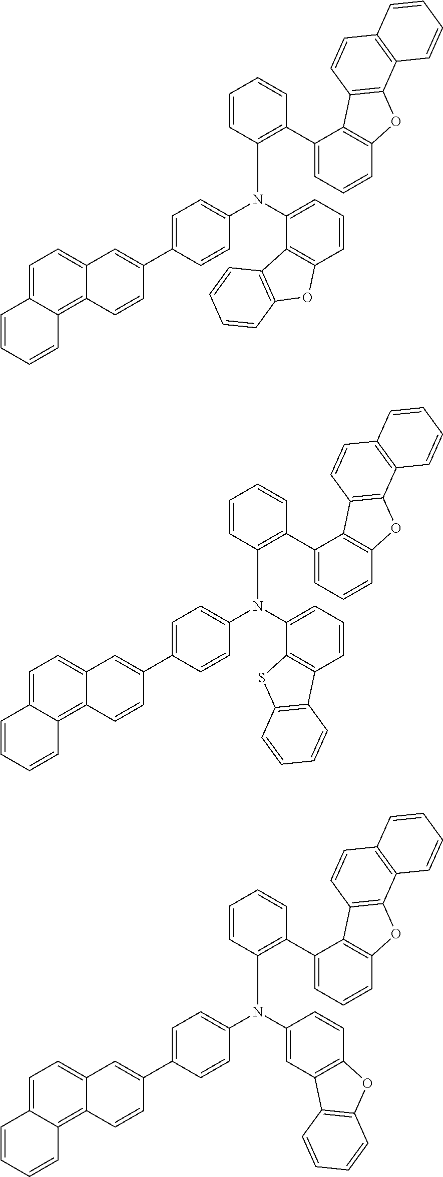 [SMILES: c1ccc(N(c2ccc(-c3ccc4c(ccc5ccccc54)c3)cc2)c2cccc3oc4ccccc4c23)c(-c2cccc3oc4c5ccccc5ccc4c23)c1 ; c1ccc(N(c2ccc(-c3ccc4c(ccc5ccccc54)c3)cc2)c2cccc3c2sc2ccccc23)c(-c2cccc3oc4c5ccccc5ccc4c23)c1 ; c1ccc(N(c2ccc(-c3ccc4c(ccc5ccccc54)c3)cc2)c2ccc3oc4ccccc4c3c2)c(-c2cccc3oc4c5ccccc5ccc4c23)c1]

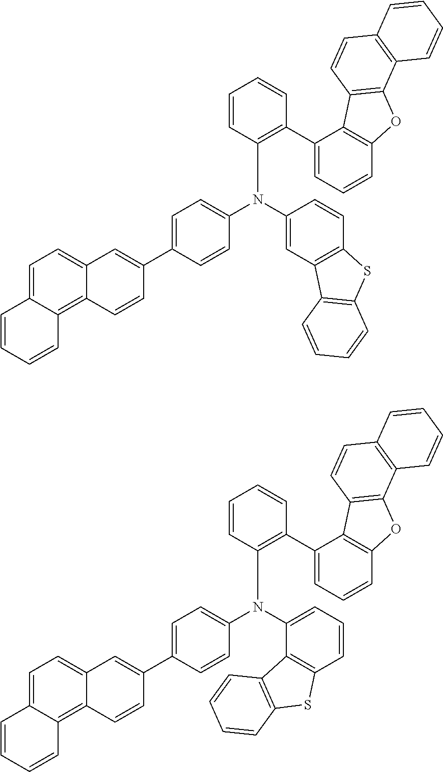 [SMILES: c1ccc(N(c2ccc(-c3ccc4c(ccc5ccccc54)c3)cc2)c2ccc3sc4ccccc4c3c2)c(-c2cccc3oc4c5ccccc5ccc4c23)c1 ; c1ccc(N(c2ccc(-c3ccc4c(ccc5ccccc54)c3)cc2)c2cccc3sc4ccccc4c23)c(-c2cccc3oc4c5ccccc5ccc4c23)c1]

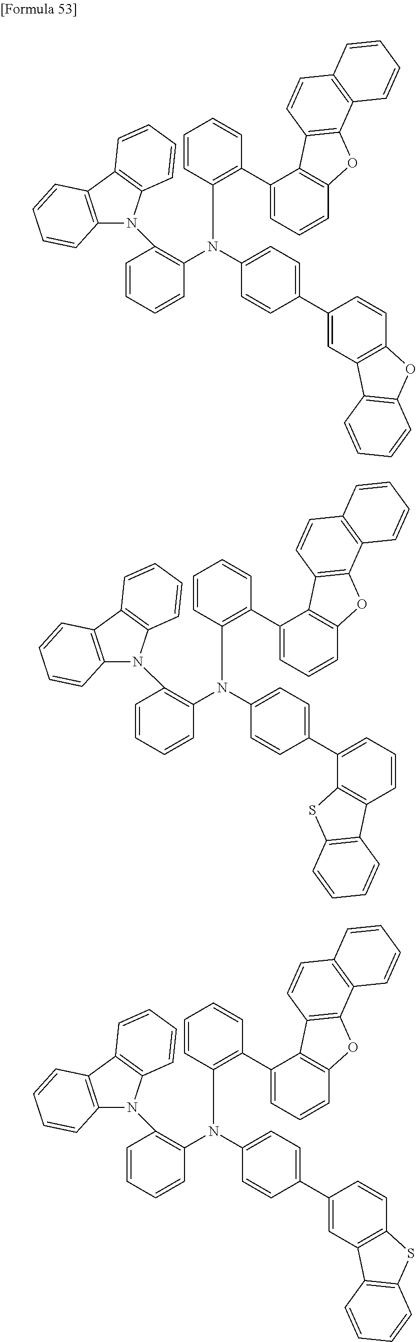

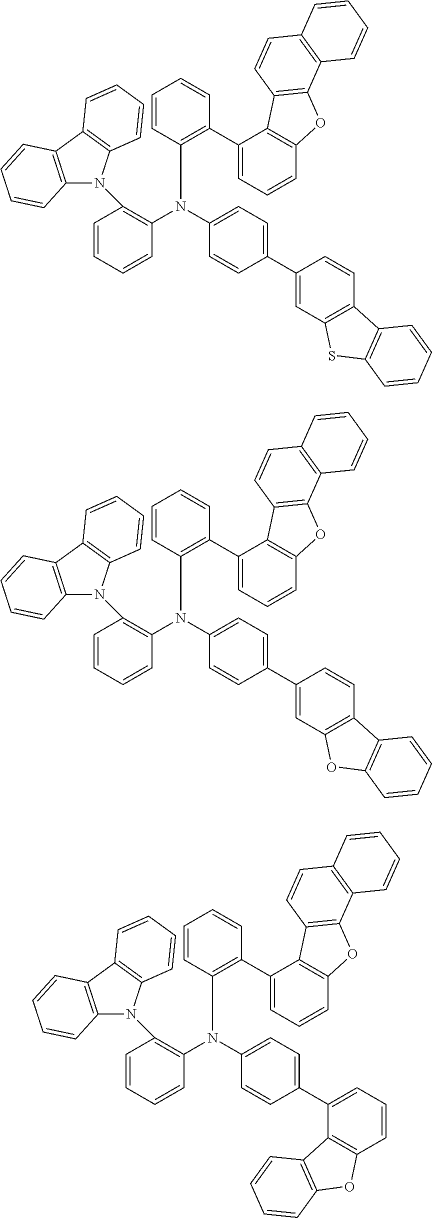

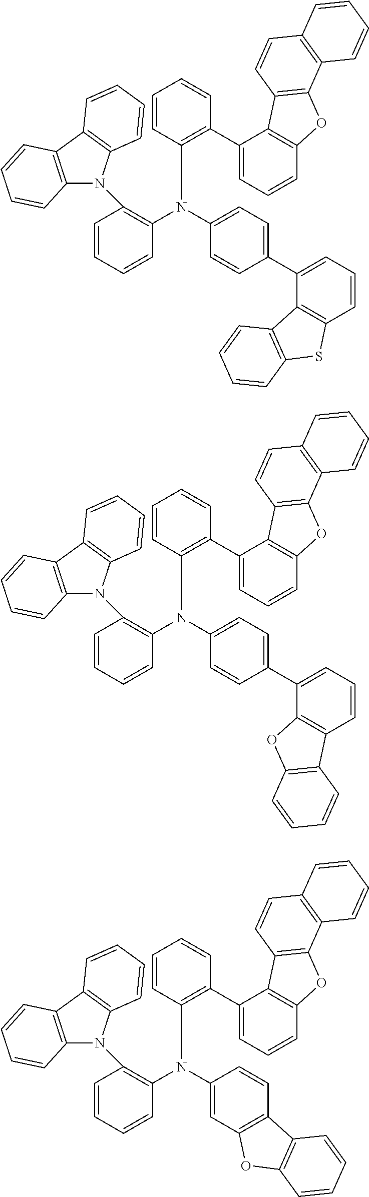

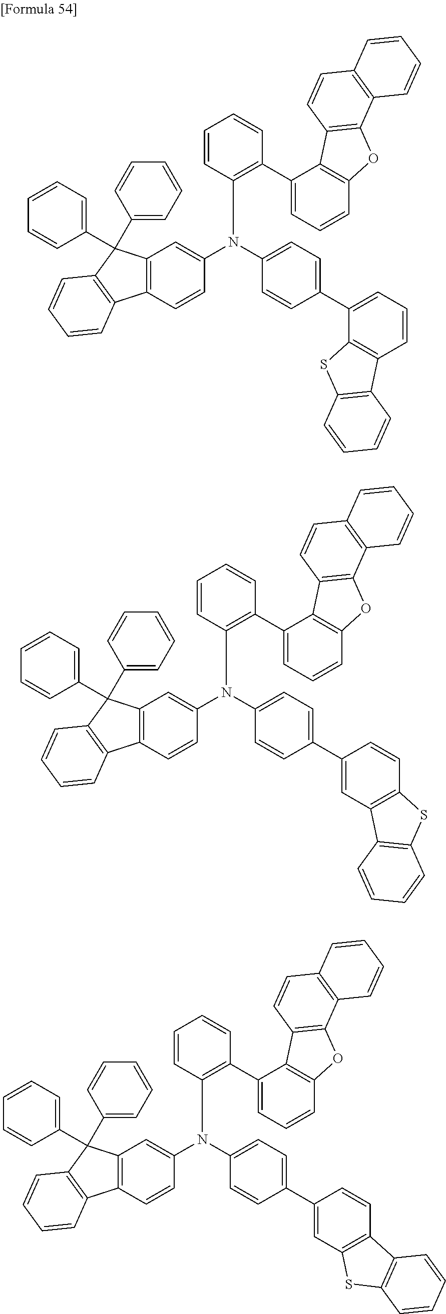

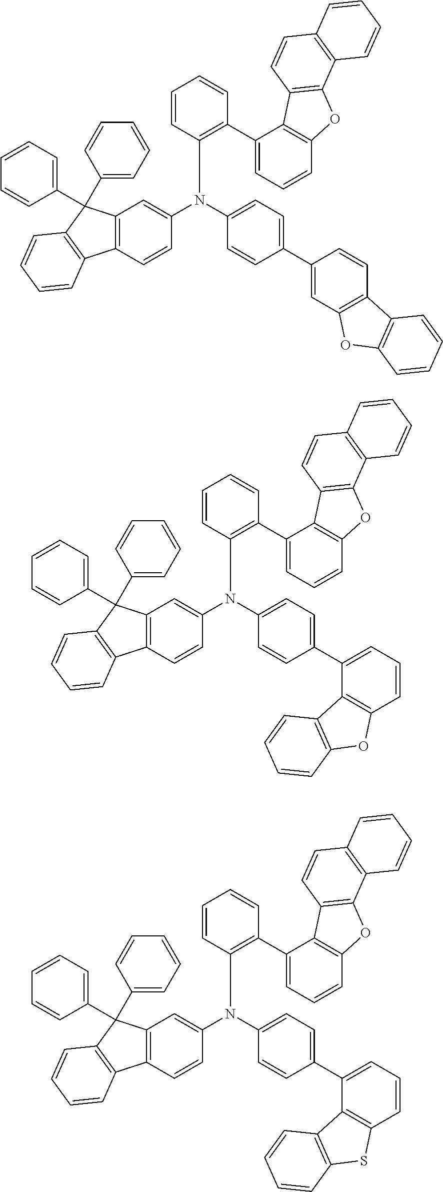

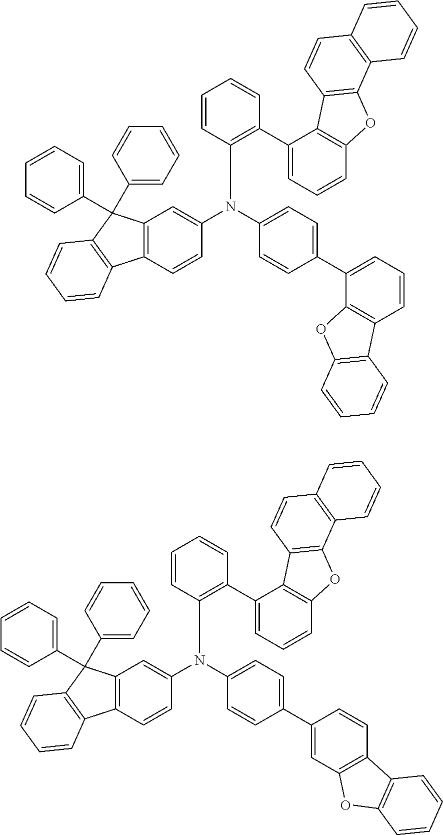 [SMILES: c1ccc(C2(c3ccccc3)c3ccccc3-c3ccc(N(c4ccc(-c5cccc6c5oc5ccccc56)cc4)c4ccccc4-c4cccc5oc6c7ccccc7ccc6c45)cc32)cc1 ; c1ccc(C2(c3ccccc3)c3ccccc3-c3ccc(N(c4ccc(-c5ccc6c(c5)oc5ccccc56)cc4)c4ccccc4-c4cccc5oc6c7ccccc7ccc6c45)cc32)cc1]

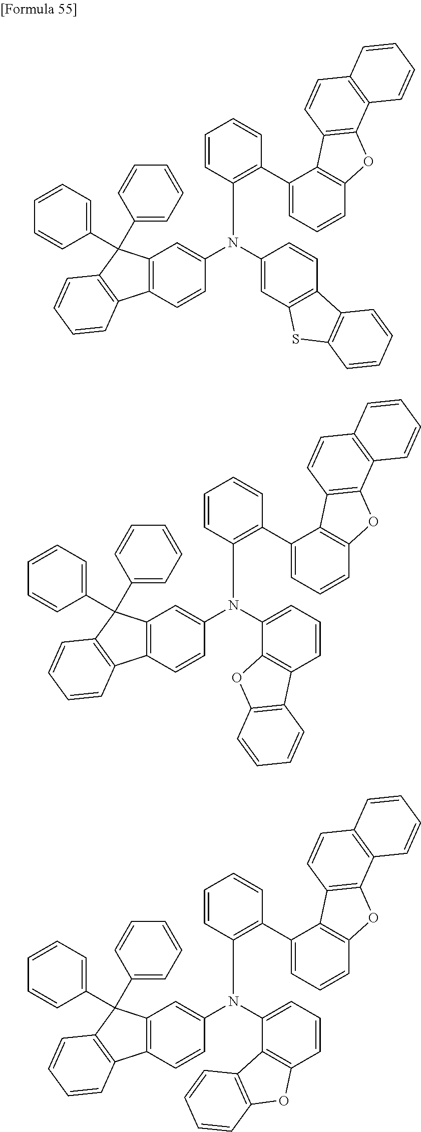

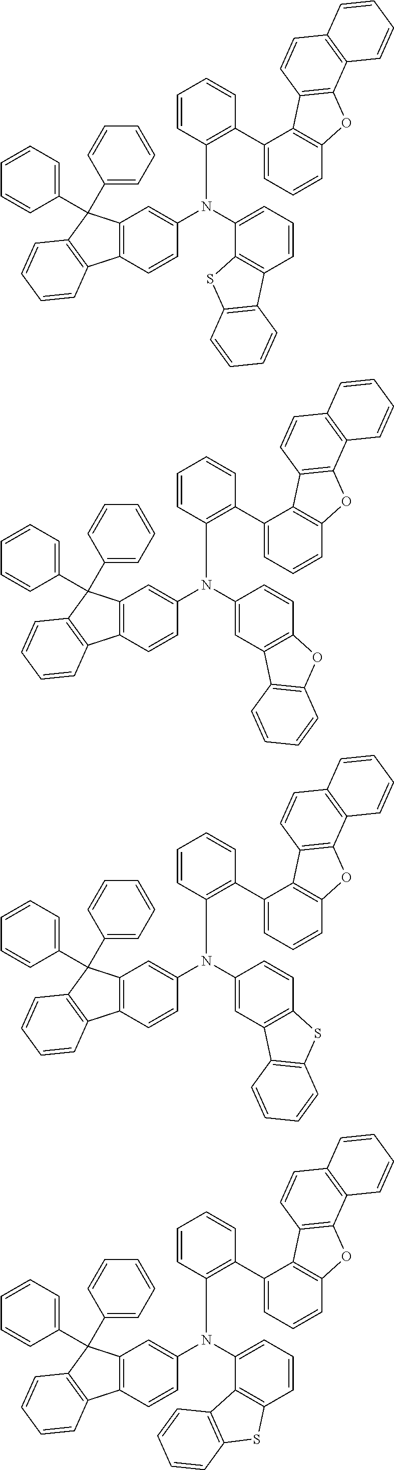

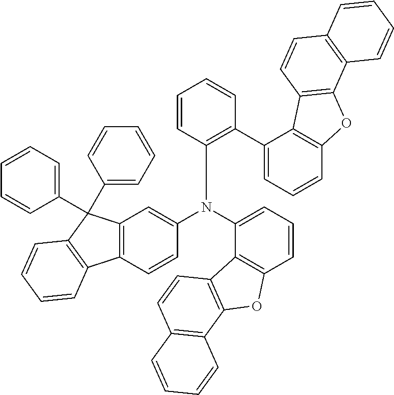 [SMILES: c1ccc(C2(c3ccccc3)c3ccccc3-c3ccc(N(c4ccccc4-c4cccc5oc6c7ccccc7ccc6c45)c4cccc5oc6c7ccccc7ccc6c45)cc32)cc1]

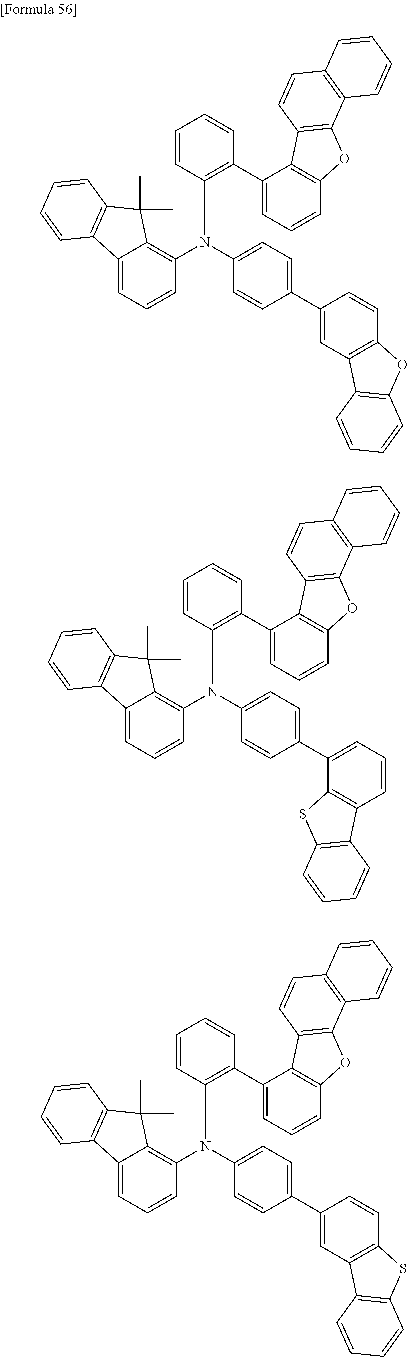

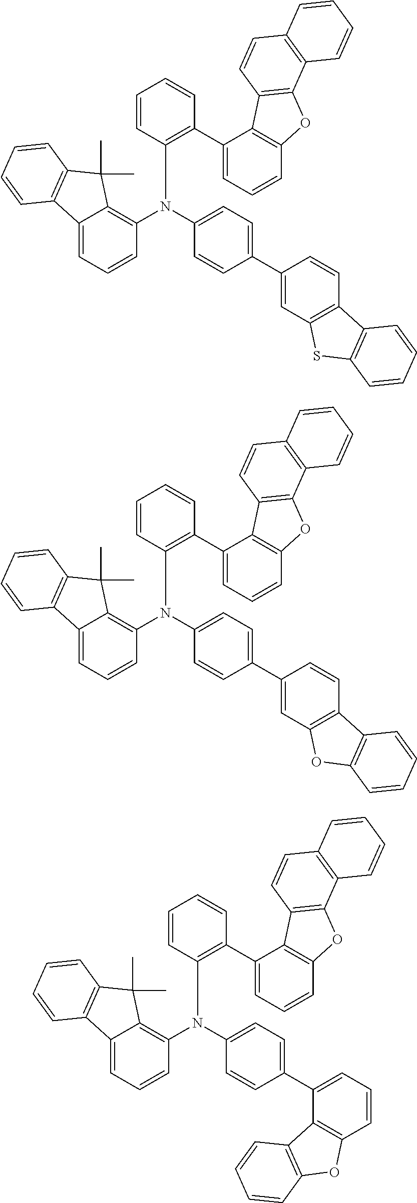

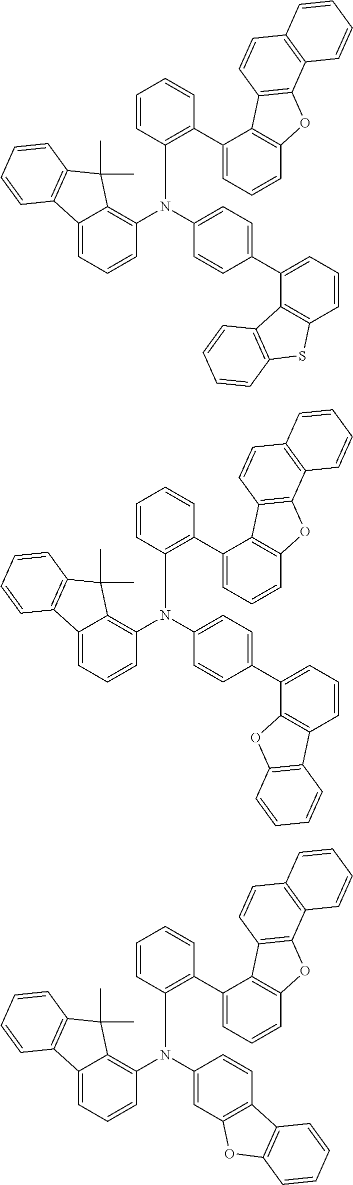

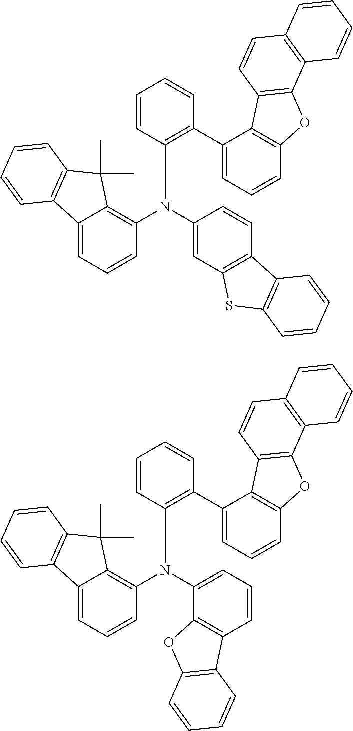

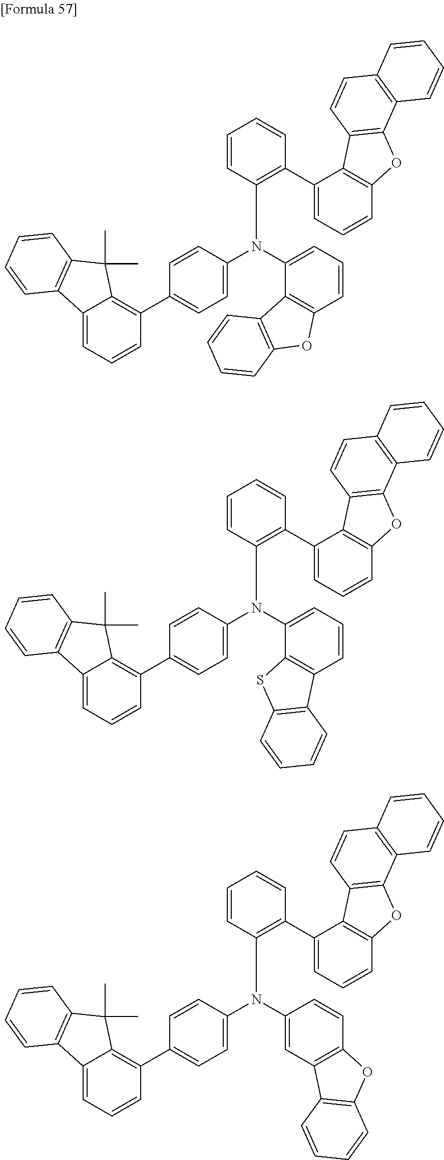 [SMILES: CC1(C)c2ccccc2-c2cccc(-c3ccc(N(c4ccccc4-c4cccc5oc6c7ccccc7ccc6c45)c4cccc5oc6ccccc6c45)cc3)c21 ; CC1(C)c2ccccc2-c2cccc(-c3ccc(N(c4ccccc4-c4cccc5oc6c7ccccc7ccc6c45)c4cccc5c4sc4ccccc45)cc3)c21 ; CC1(C)c2ccccc2-c2cccc(-c3ccc(N(c4ccc5oc6ccccc6c5c4)c4ccccc4-c4cccc5oc6c7ccccc7ccc6c45)cc3)c21]

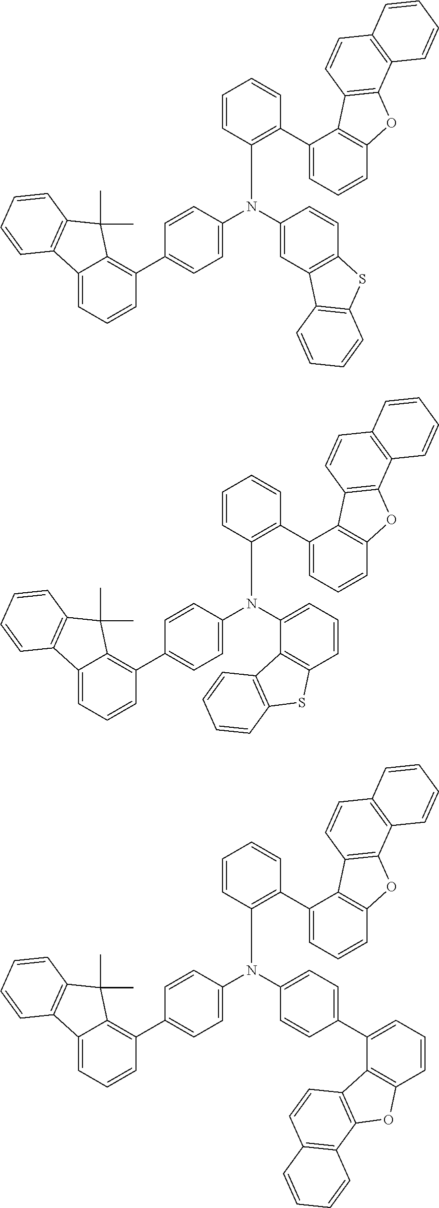 [SMILES: CC1(C)c2ccccc2-c2cccc(-c3ccc(N(c4ccc5sc6ccccc6c5c4)c4ccccc4-c4cccc5oc6c7ccccc7ccc6c45)cc3)c21 ; CC1(C)c2ccccc2-c2cccc(-c3ccc(N(c4ccccc4-c4cccc5oc6c7ccccc7ccc6c45)c4cccc5sc6ccccc6c45)cc3)c21 ; CC1(C)c2ccccc2-c2cccc(-c3ccc(N(c4ccc(-c5cccc6oc7c8ccccc8ccc7c56)cc4)c4ccccc4-c4cccc5oc6c7ccccc7ccc6c45)cc3)c21]

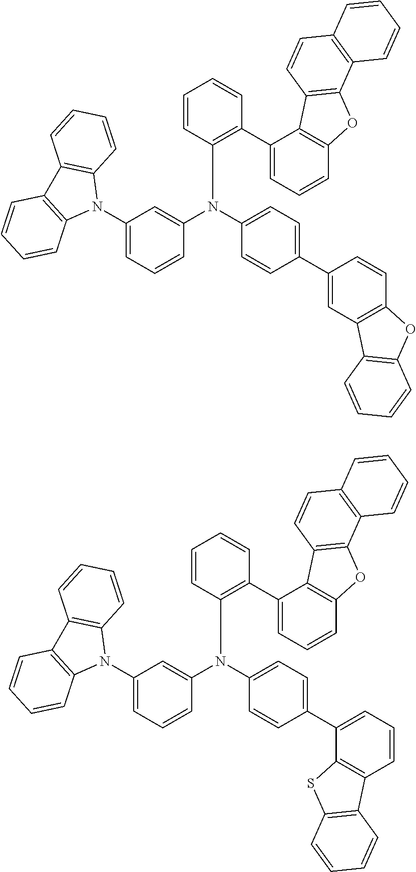

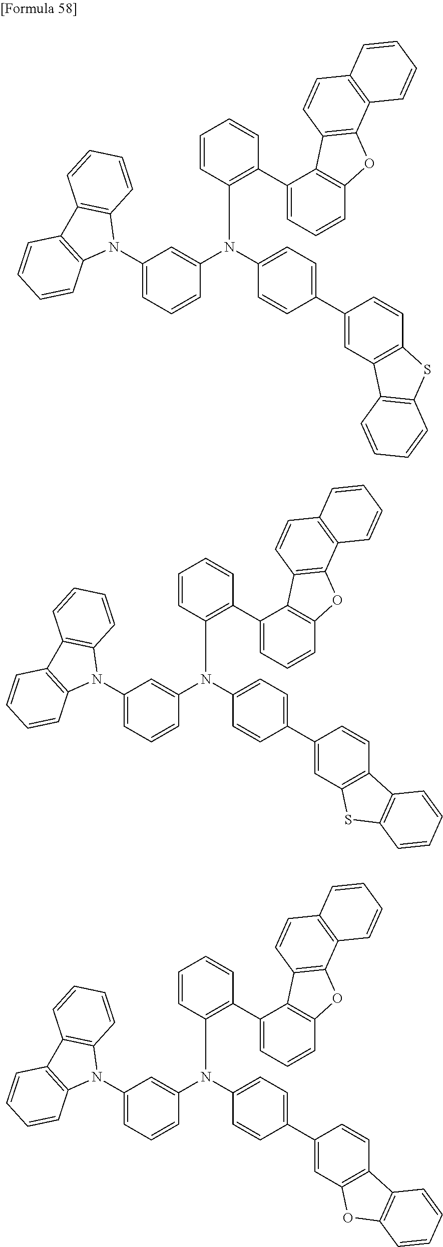

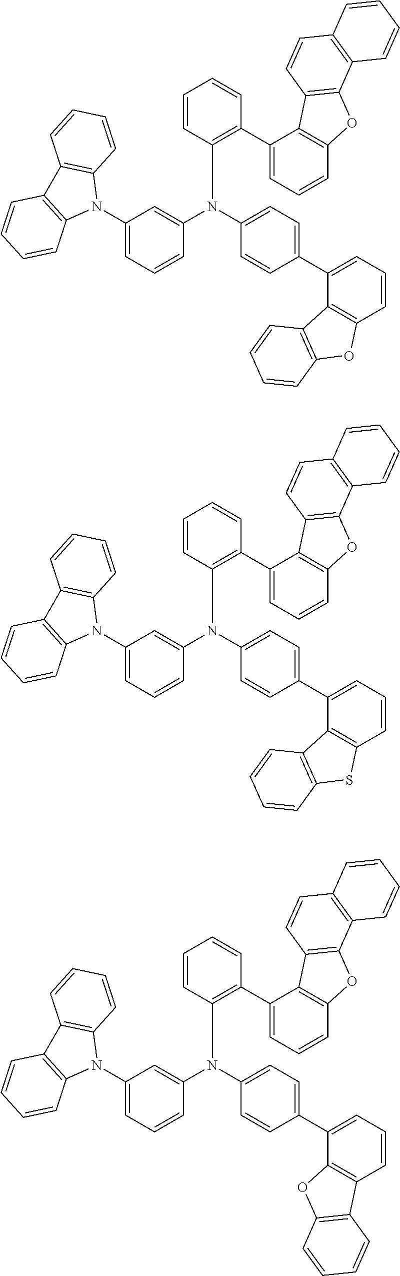 [SMILES: c1cc(N(c2ccc(-c3cccc4oc5ccccc5c34)cc2)c2ccccc2-c2cccc3oc4c5ccccc5ccc4c23)cc(-n2c3ccccc3c3ccccc32)c1 ; c1cc(N(c2ccc(-c3cccc4sc5ccccc5c34)cc2)c2ccccc2-c2cccc3oc4c5ccccc5ccc4c23)cc(-n2c3ccccc3c3ccccc32)c1 ; c1cc(N(c2ccc(-c3cccc4c3oc3ccccc34)cc2)c2ccccc2-c2cccc3oc4c5ccccc5ccc4c23)cc(-n2c3ccccc3c3ccccc32)c1]

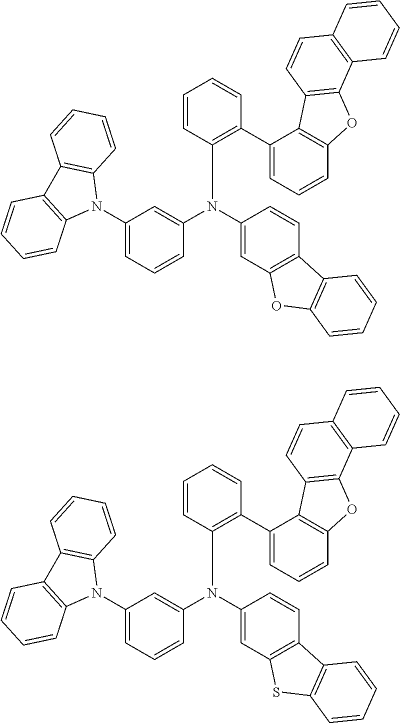 [SMILES: c1cc(N(c2ccc3c(c2)oc2ccccc23)c2ccccc2-c2cccc3oc4c5ccccc5ccc4c23)cc(-n2c3ccccc3c3ccccc32)c1 ; c1cc(N(c2ccc3c(c2)sc2ccccc23)c2ccccc2-c2cccc3oc4c5ccccc5ccc4c23)cc(-n2c3ccccc3c3ccccc32)c1]

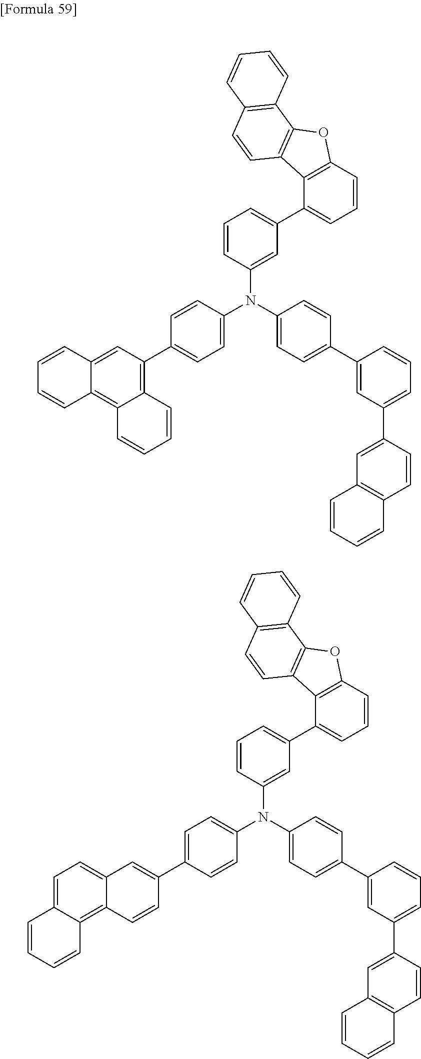 [SMILES: c1cc(-c2ccc(N(c3ccc(-c4cc5ccccc5c5ccccc45)cc3)c3cccc(-c4cccc5oc6c7ccccc7ccc6c45)c3)cc2)cc(-c2ccc3ccccc3c2)c1 ; c1cc(-c2ccc(N(c3ccc(-c4ccc5c(ccc6ccccc65)c4)cc3)c3cccc(-c4cccc5oc6c7ccccc7ccc6c45)c3)cc2)cc(-c2ccc3ccccc3c2)c1]

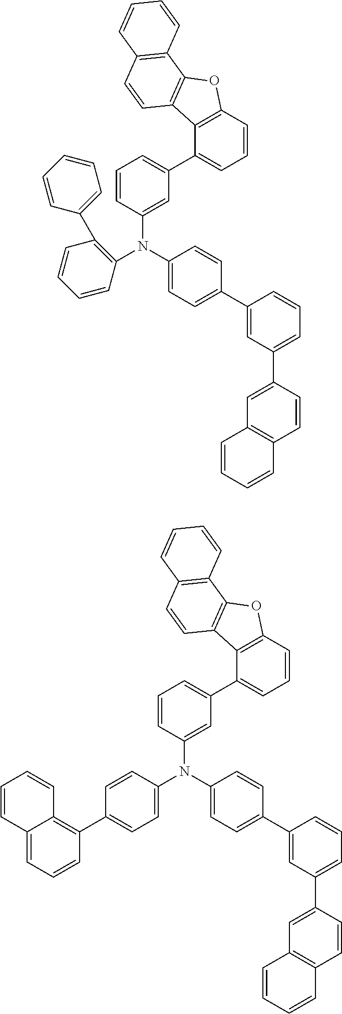 [SMILES: c1ccc(-c2ccccc2N(c2ccc(-c3cccc(-c4ccc5ccccc5c4)c3)cc2)c2cccc(-c3cccc4oc5c6ccccc6ccc5c34)c2)cc1 ; c1cc(-c2ccc(N(c3ccc(-c4cccc5ccccc45)cc3)c3cccc(-c4cccc5oc6c7ccccc7ccc6c45)c3)cc2)cc(-c2ccc3ccccc3c2)c1]

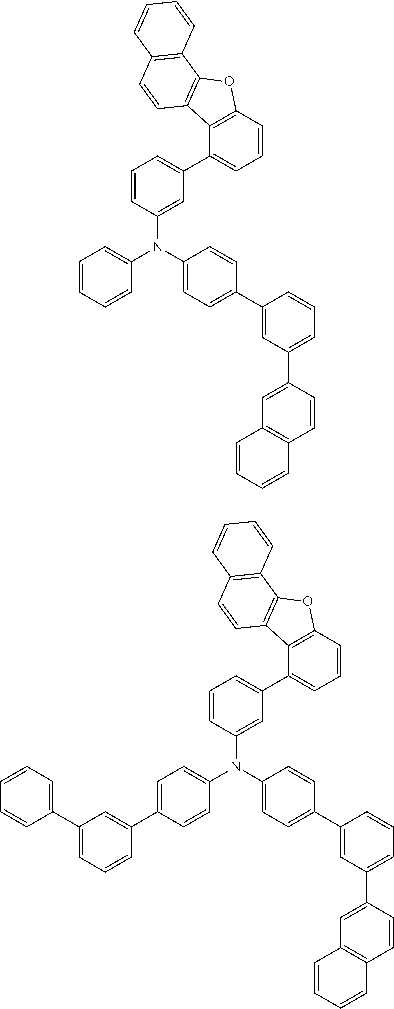

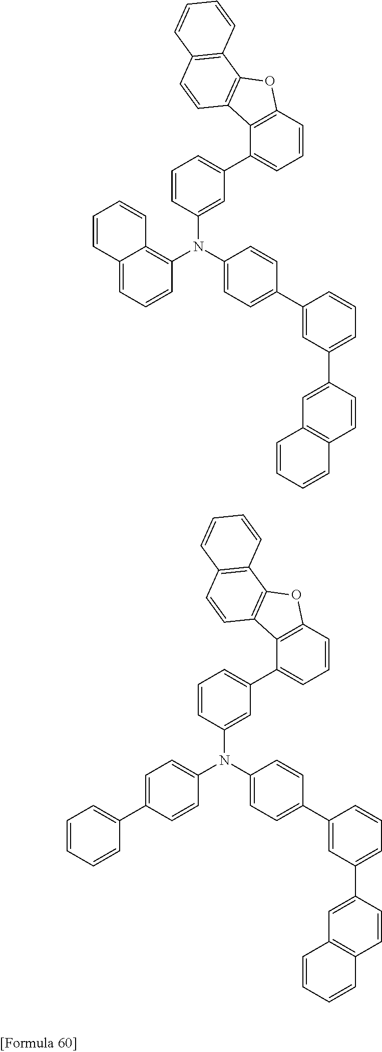 [SMILES: c1cc(-c2ccc(N(c3cccc(-c4cccc5oc6c7ccccc7ccc6c45)c3)c3cccc4ccccc34)cc2)cc(-c2ccc3ccccc3c2)c1 ; c1ccc(-c2ccc(N(c3ccc(-c4cccc(-c5ccc6ccccc6c5)c4)cc3)c3cccc(-c4cccc5oc6c7ccccc7ccc6c45)c3)cc2)cc1]

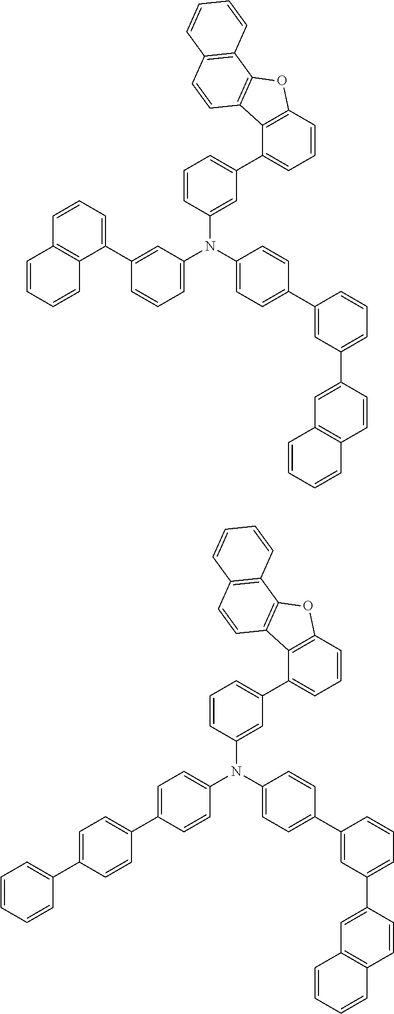 [SMILES: c1cc(-c2ccc(N(c3cccc(-c4cccc5ccccc45)c3)c3cccc(-c4cccc5oc6c7ccccc7ccc6c45)c3)cc2)cc(-c2ccc3ccccc3c2)c1 ; c1ccc(-c2ccc(-c3ccc(N(c4ccc(-c5cccc(-c6ccc7ccccc7c6)c5)cc4)c4cccc(-c5cccc6oc7c8ccccc8ccc7c56)c4)cc3)cc2)cc1]

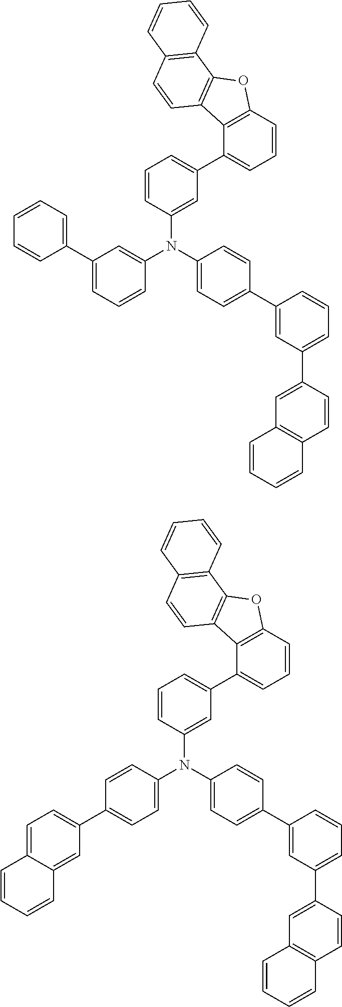 [SMILES: c1ccc(-c2cccc(N(c3ccc(-c4cccc(-c5ccc6ccccc6c5)c4)cc3)c3cccc(-c4cccc5oc6c7ccccc7ccc6c45)c3)c2)cc1 ; c1cc(-c2ccc(N(c3ccc(-c4ccc5ccccc5c4)cc3)c3cccc(-c4cccc5oc6c7ccccc7ccc6c45)c3)cc2)cc(-c2ccc3ccccc3c2)c1]

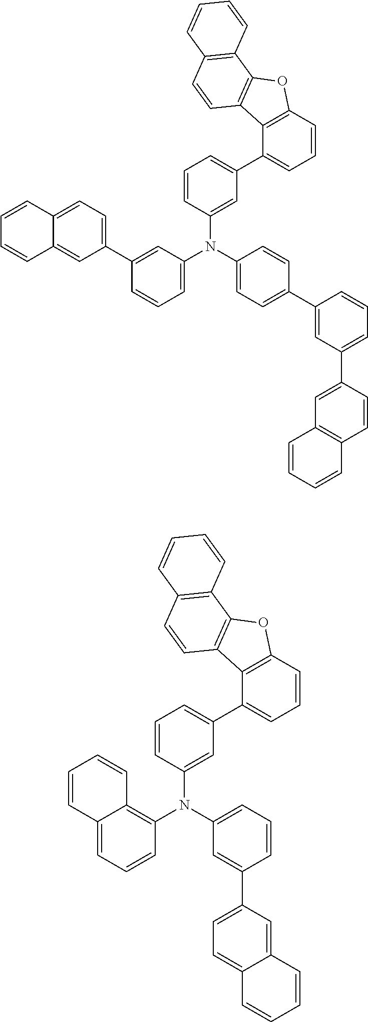 [SMILES: c1cc(-c2ccc(N(c3cccc(-c4ccc5ccccc5c4)c3)c3cccc(-c4cccc5oc6c7ccccc7ccc6c45)c3)cc2)cc(-c2ccc3ccccc3c2)c1 ; c1cc(-c2ccc3ccccc3c2)cc(N(c2cccc(-c3cccc4oc5c6ccccc6ccc5c34)c2)c2cccc3ccccc23)c1]

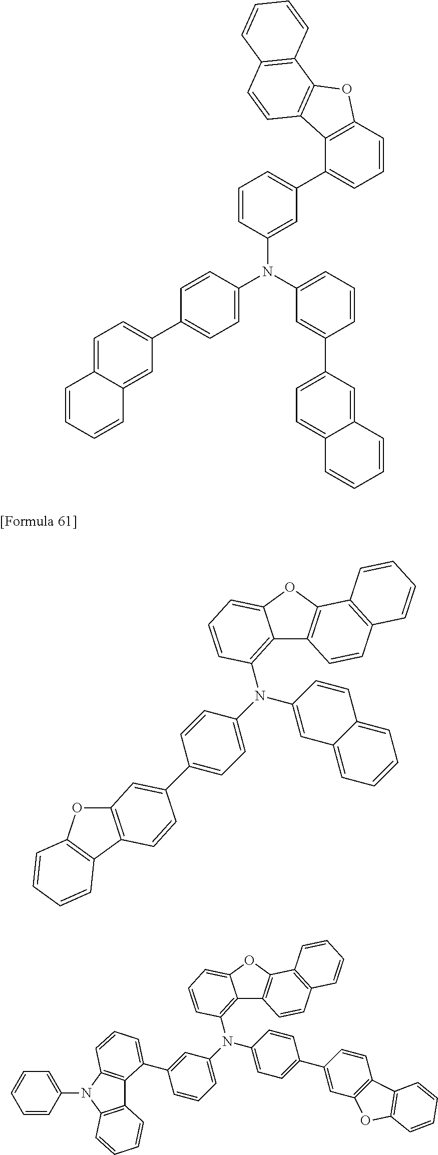 [SMILES: c1cc(-c2ccc3ccccc3c2)cc(N(c2ccc(-c3ccc4ccccc4c3)cc2)c2cccc(-c3cccc4oc5c6ccccc6ccc5c34)c2)c1 ; c1ccc2cc(N(c3ccc(-c4ccc5c(c4)oc4ccccc45)cc3)c3cccc4oc5c6ccccc6ccc5c34)ccc2c1 ; c1ccc(-n2c3ccccc3c3c(-c4cccc(N(c5ccc(-c6ccc7c(c6)oc6ccccc67)cc5)c5cccc6oc7c8ccccc8ccc7c56)c4)cccc32)cc1]

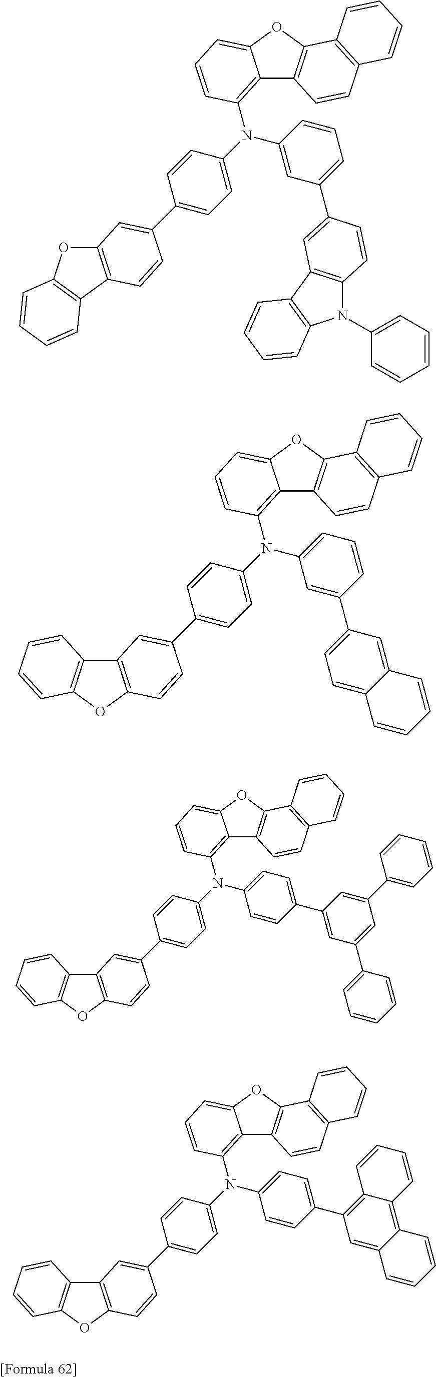

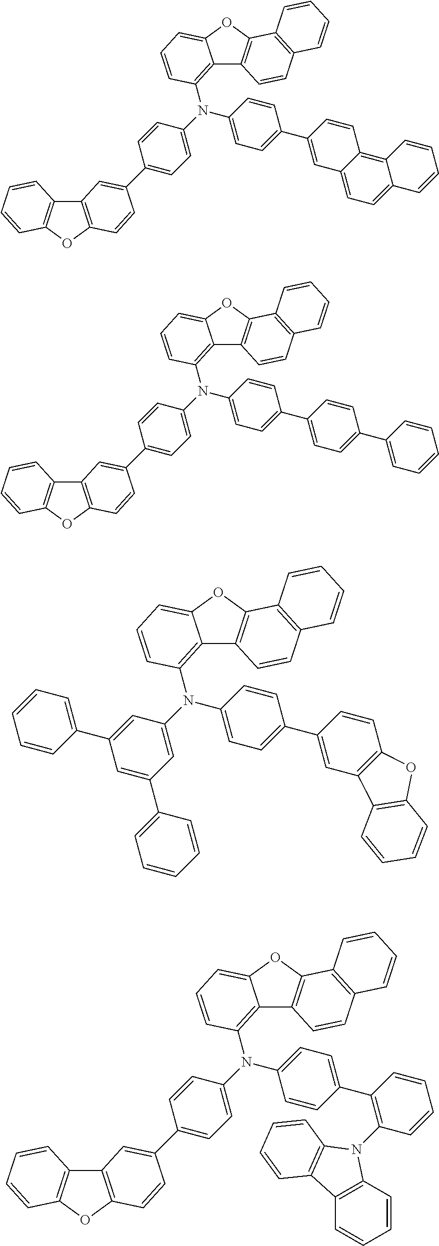 [SMILES: c1ccc2c(c1)ccc1cc(-c3ccc(N(c4ccc(-c5ccc6oc7ccccc7c6c5)cc4)c4cccc5oc6c7ccccc7ccc6c45)cc3)ccc12 ; c1ccc(-c2ccc(-c3ccc(N(c4ccc(-c5ccc6oc7ccccc7c6c5)cc4)c4cccc5oc6c7ccccc7ccc6c45)cc3)cc2)cc1 ; c1ccc(-c2cc(-c3ccccc3)cc(N(c3ccc(-c4ccc5oc6ccccc6c5c4)cc3)c3cccc4oc5c6ccccc6ccc5c34)c2)cc1 ; c1ccc(-n2c3ccccc3c3ccccc32)c(-c2ccc(N(c3ccc(-c4ccc5oc6ccccc6c5c4)cc3)c3cccc4oc5c6ccccc6ccc5c34)cc2)c1]

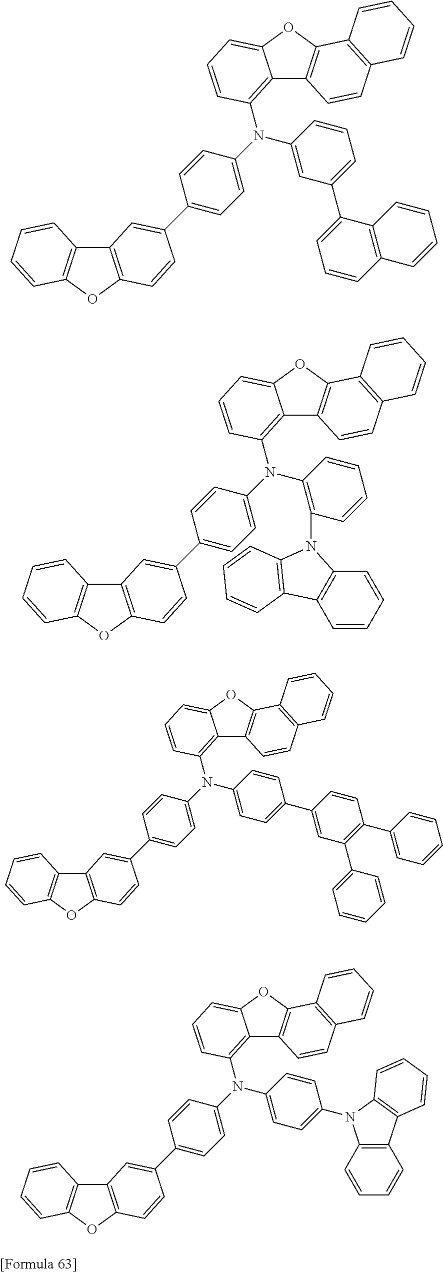 [SMILES: c1cc(-c2cccc3ccccc23)cc(N(c2ccc(-c3ccc4oc5ccccc5c4c3)cc2)c2cccc3oc4c5ccccc5ccc4c23)c1 ; c1ccc(-n2c3ccccc3c3ccccc32)c(N(c2ccc(-c3ccc4oc5ccccc5c4c3)cc2)c2cccc3oc4c5ccccc5ccc4c23)c1 ; c1ccc(-c2ccc(-c3ccc(N(c4ccc(-c5ccc6oc7ccccc7c6c5)cc4)c4cccc5oc6c7ccccc7ccc6c45)cc3)cc2-c2ccccc2)cc1 ; c1ccc2c(c1)ccc1c2oc2cccc(N(c3ccc(-c4ccc5oc6ccccc6c5c4)cc3)c3ccc(-n4c5ccccc5c5ccccc54)cc3)c21]

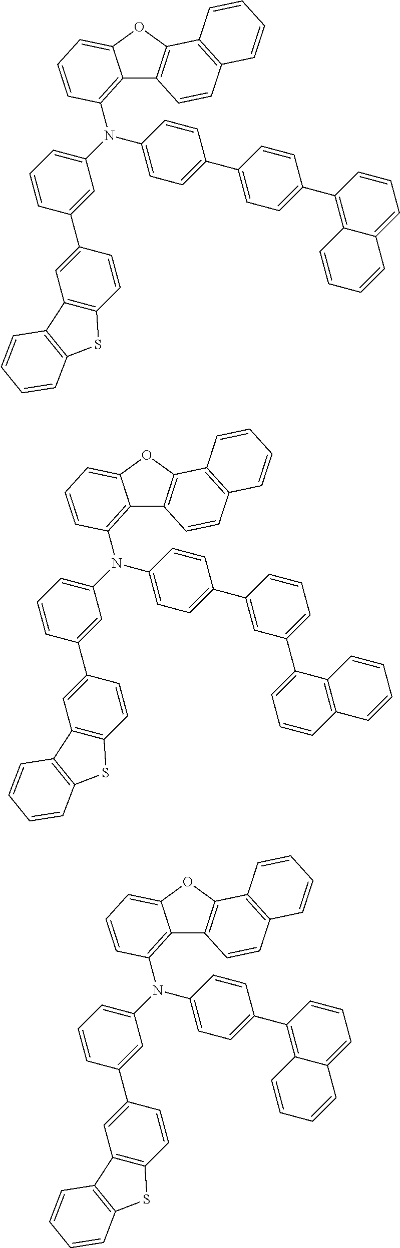 [SMILES: c1cc(-c2ccc3sc4ccccc4c3c2)cc(N(c2ccc(-c3ccc(-c4cccc5ccccc45)cc3)cc2)c2cccc3oc4c5ccccc5ccc4c23)c1 ; c1cc(-c2ccc(N(c3cccc(-c4ccc5sc6ccccc6c5c4)c3)c3cccc4oc5c6ccccc6ccc5c34)cc2)cc(-c2cccc3ccccc23)c1 ; c1cc(-c2ccc3sc4ccccc4c3c2)cc(N(c2ccc(-c3cccc4ccccc34)cc2)c2cccc3oc4c5ccccc5ccc4c23)c1]

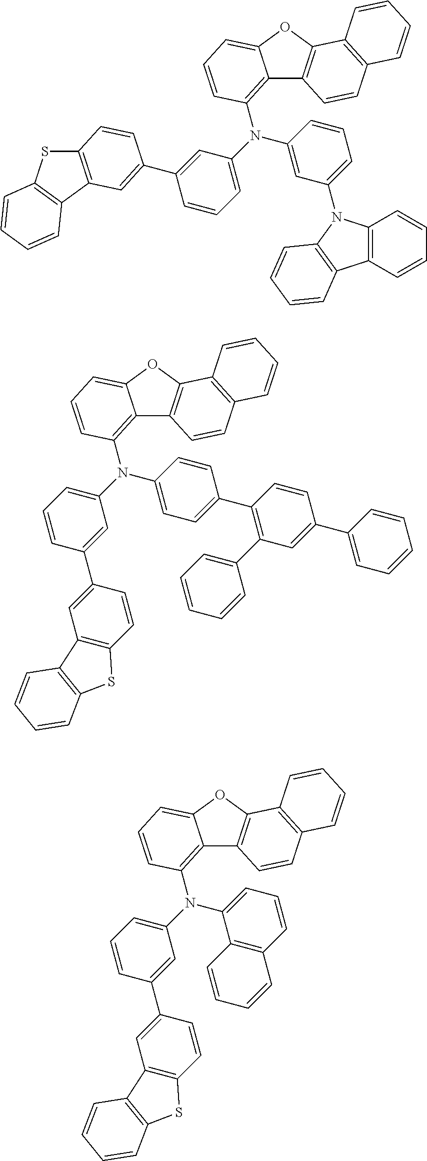 [SMILES: c1cc(-c2ccc3sc4ccccc4c3c2)cc(N(c2cccc(-n3c4ccccc4c4ccccc43)c2)c2cccc3oc4c5ccccc5ccc4c23)c1 ; c1ccc(-c2ccc(-c3ccc(N(c4cccc(-c5ccc6sc7ccccc7c6c5)c4)c4cccc5oc6c7ccccc7ccc6c45)cc3)c(-c3ccccc3)c2)cc1 ; c1cc(-c2ccc3sc4ccccc4c3c2)cc(N(c2cccc3ccccc23)c2cccc3oc4c5ccccc5ccc4c23)c1]

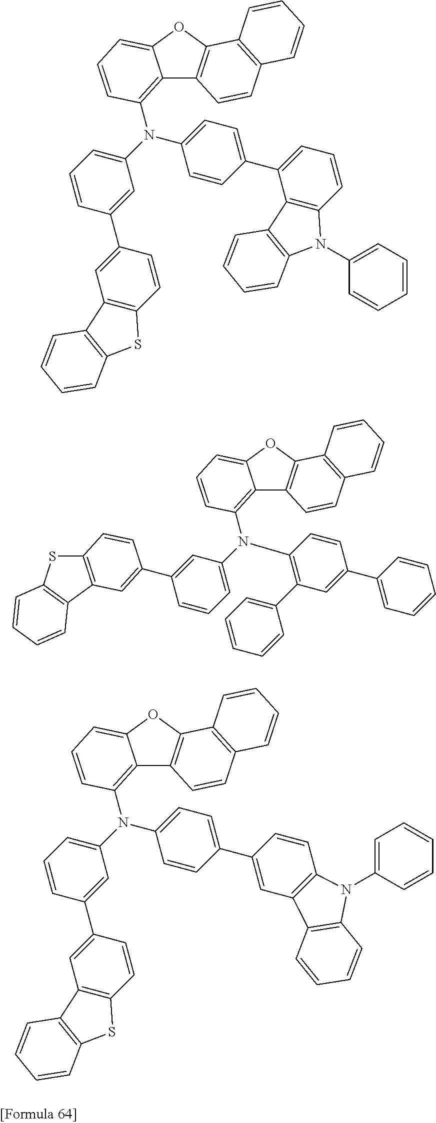

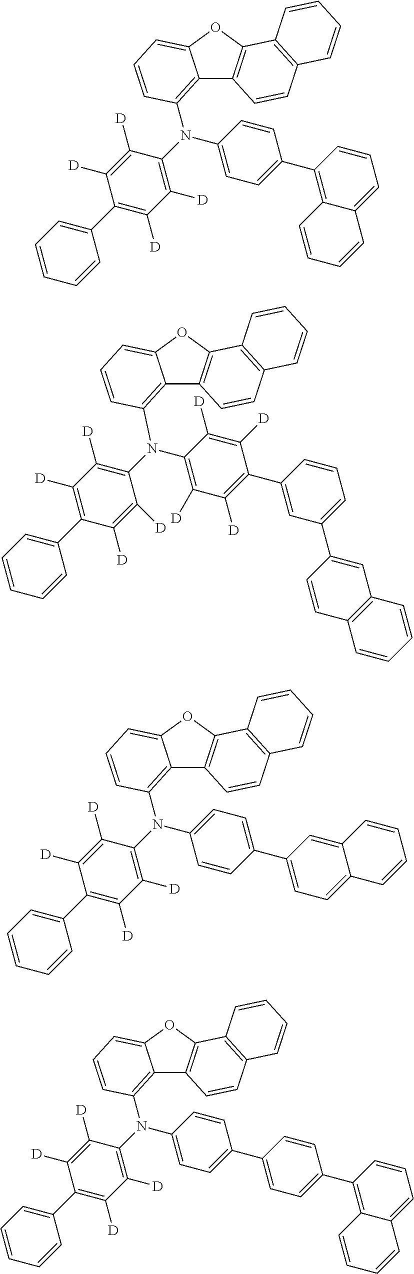 [SMILES: [2H]c1c([2H])c(N(c2ccc(-c3cccc4ccccc34)cc2)c2cccc3oc4c5ccccc5ccc4c23)c([2H])c([2H])c1-c1ccccc1 ; [2H]c1c([2H])c(N(c2c([2H])c([2H])c(-c3cccc(-c4ccc5ccccc5c4)c3)c([2H])c2[2H])c2cccc3oc4c5ccccc5ccc4c23)c([2H])c([2H])c1-c1ccccc1 ; [2H]c1c([2H])c(N(c2ccc(-c3ccc4ccccc4c3)cc2)c2cccc3oc4c5ccccc5ccc4c23)c([2H])c([2H])c1-c1ccccc1 ; [2H]c1c([2H])c(N(c2ccc(-c3ccc(-c4cccc5ccccc45)cc3)cc2)c2cccc3oc4c5ccccc5ccc4c23)c([2H])c([2H])c1-c1ccccc1]

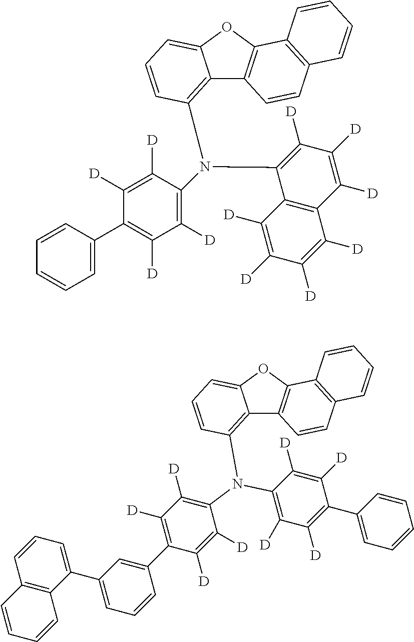 [SMILES: [2H]c1c([2H])c(N(c2c([2H])c([2H])c([2H])c3c([2H])c([2H])c([2H])c([2H])c23)c2cccc3oc4c5ccccc5ccc4c23)c([2H])c([2H])c1-c1ccccc1 ; [2H]c1c([2H])c(N(c2c([2H])c([2H])c(-c3cccc(-c4cccc5ccccc45)c3)c([2H])c2[2H])c2cccc3oc4c5ccccc5ccc4c23)c([2H])c([2H])c1-c1ccccc1]

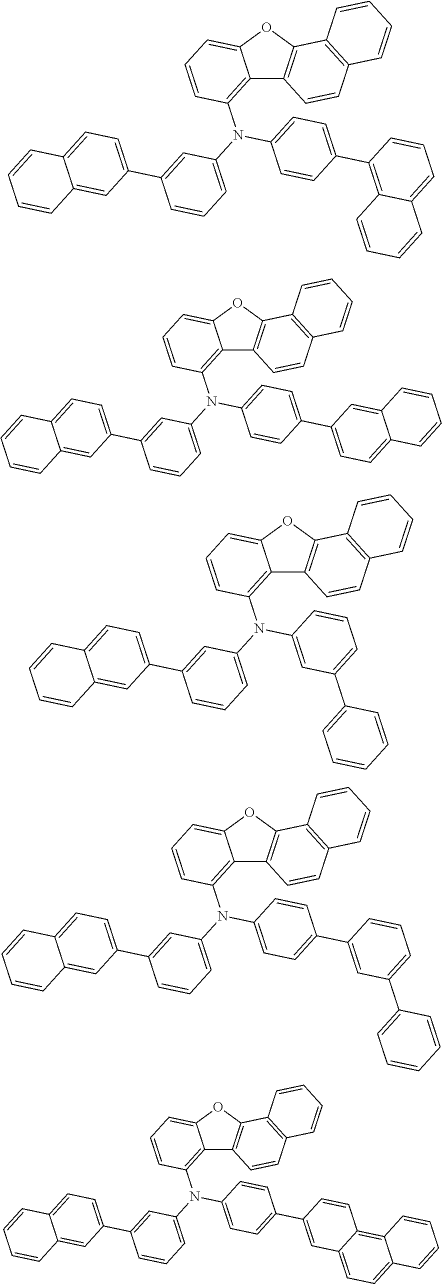

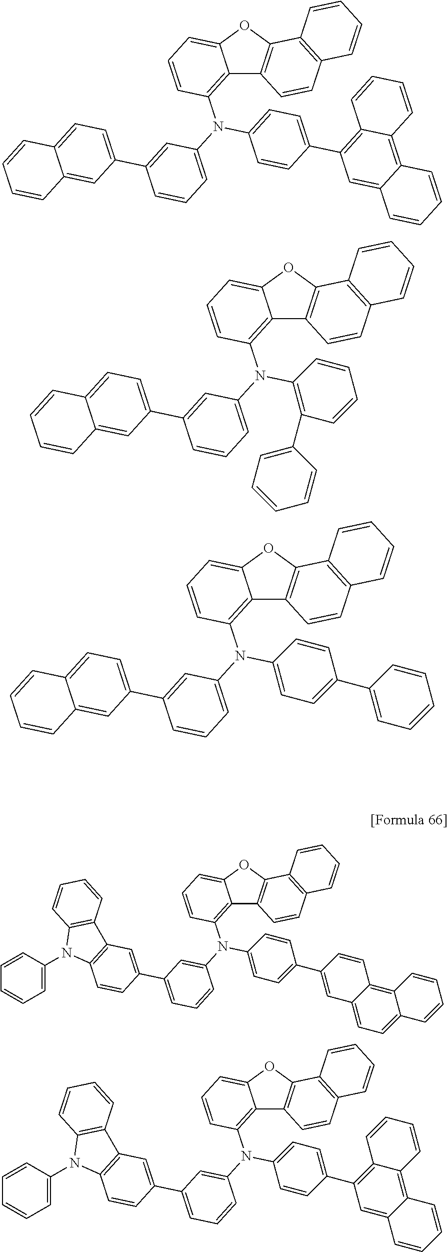

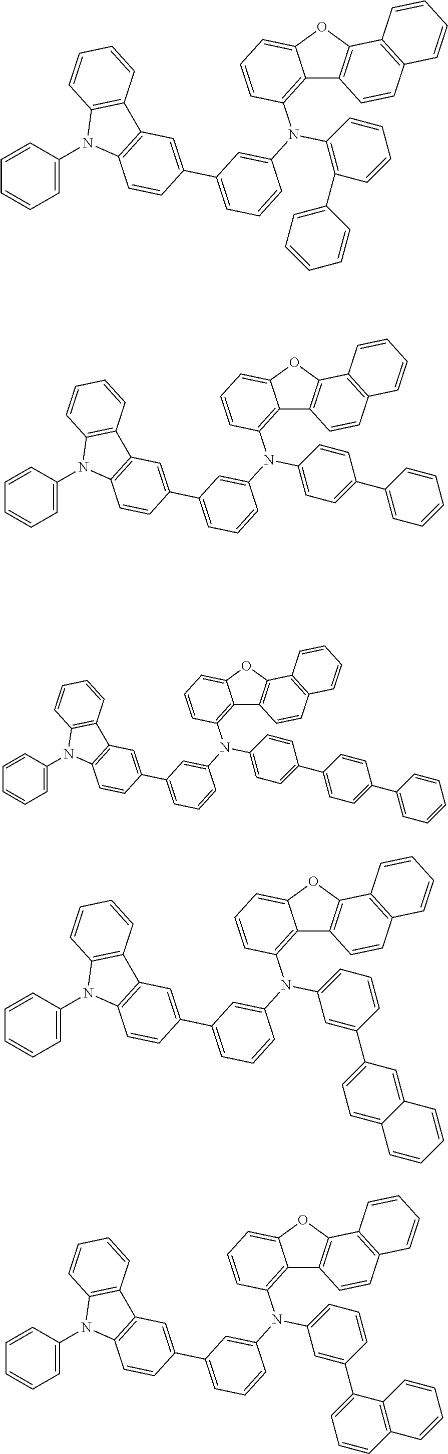

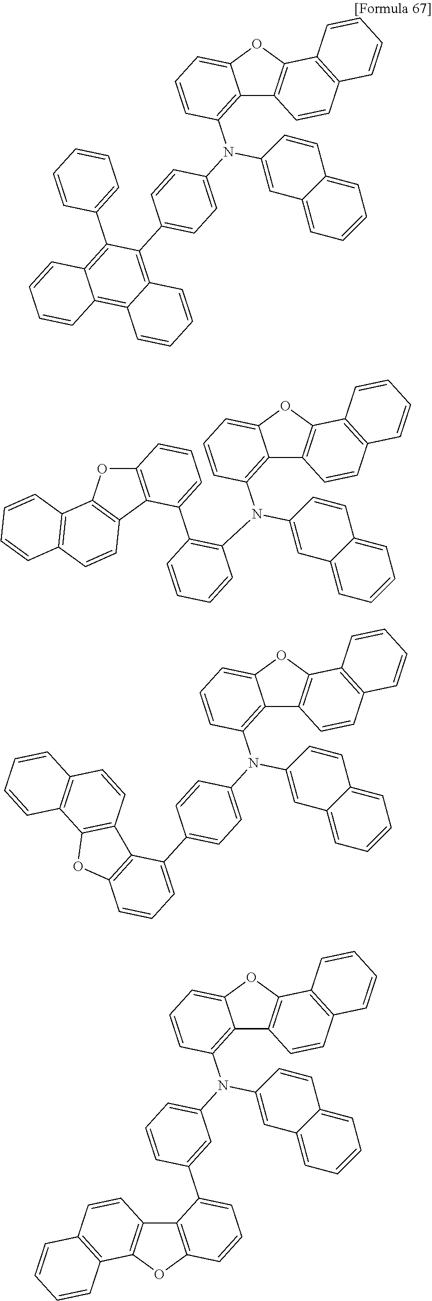

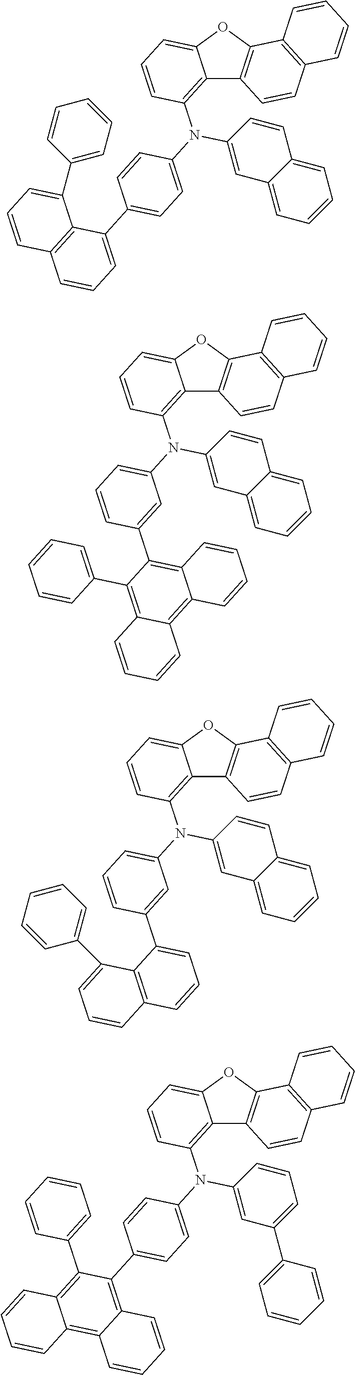 [SMILES: c1ccc(-c2cccc3cccc(-c4ccc(N(c5ccc6ccccc6c5)c5cccc6oc7c8ccccc8ccc7c56)cc4)c23)cc1 ; c1ccc(-c2c(-c3cccc(N(c4ccc5ccccc5c4)c4cccc5oc6c7ccccc7ccc6c45)c3)c3ccccc3c3ccccc23)cc1 ; c1ccc(-c2cccc3cccc(-c4cccc(N(c5ccc6ccccc6c5)c5cccc6oc7c8ccccc8ccc7c56)c4)c23)cc1 ; c1ccc(-c2cccc(N(c3ccc(-c4c(-c5ccccc5)c5ccccc5c5ccccc45)cc3)c3cccc4oc5c6ccccc6ccc5c34)c2)cc1]

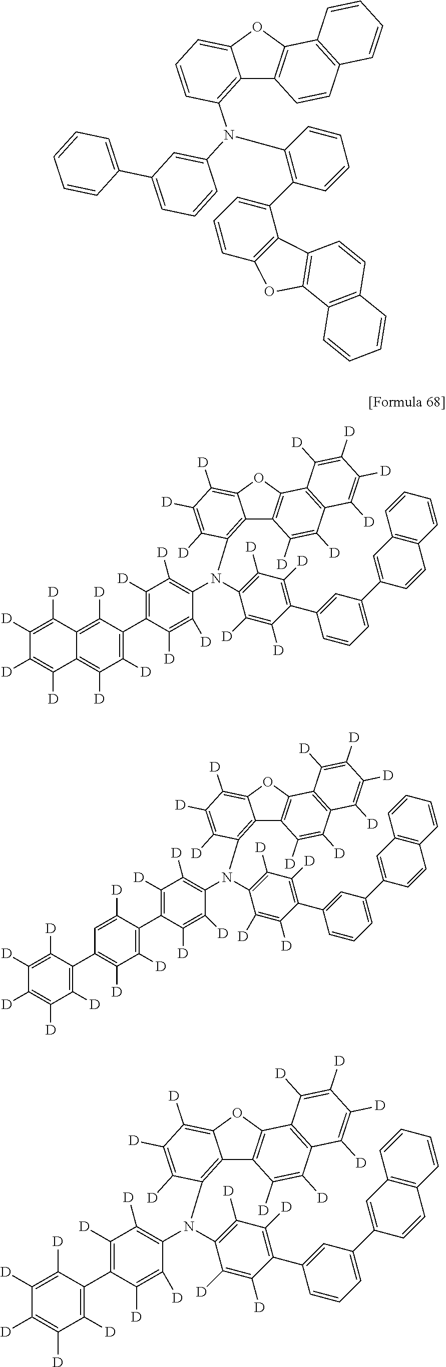

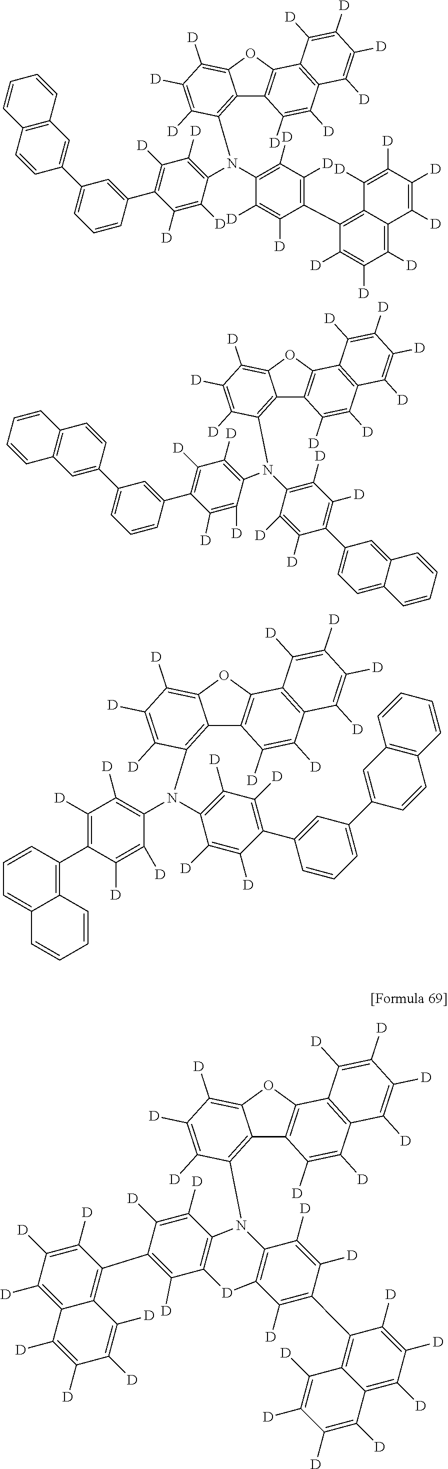

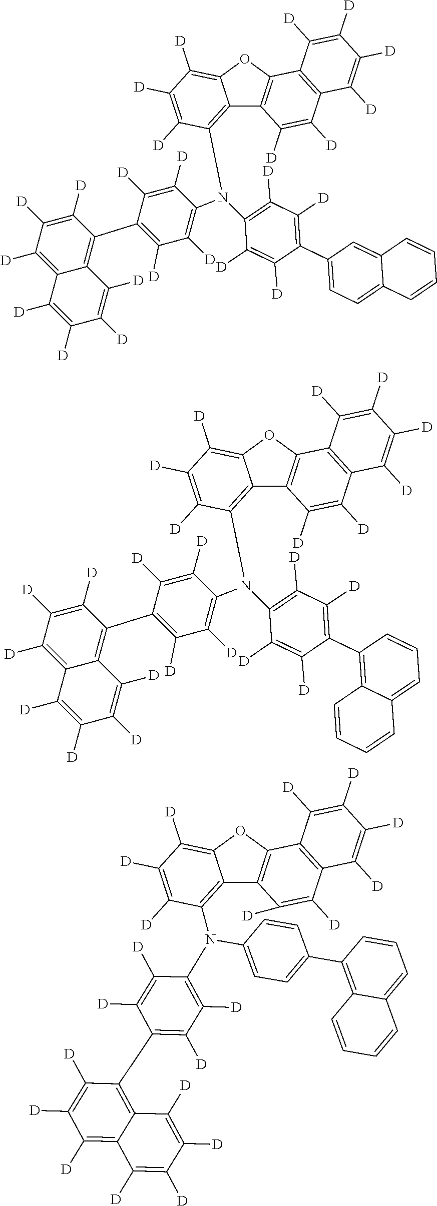

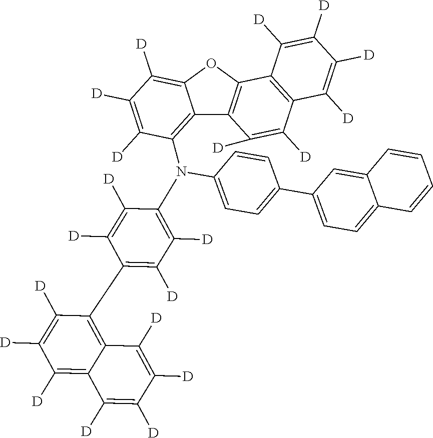 [SMILES: [2H]c1c([2H])c(N(c2ccc(-c3ccc4ccccc4c3)cc2)c2c([2H])c([2H])c([2H])c3oc4c5c([2H])c([2H])c([2H])c([2H])c5c([2H])c([2H])c4c23)c([2H])c([2H])c1-c1c([2H])c([2H])c([2H])c2c([2H])c([2H])c([2H])c([2H])c12]

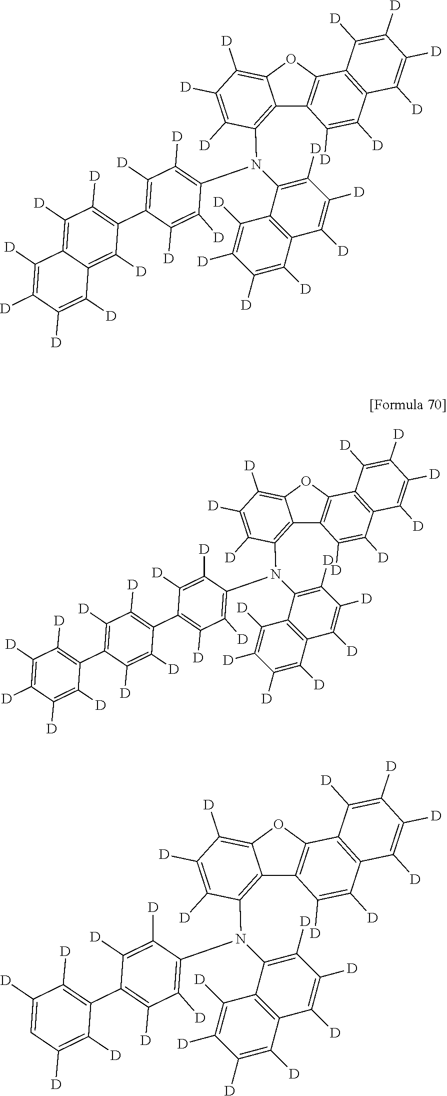

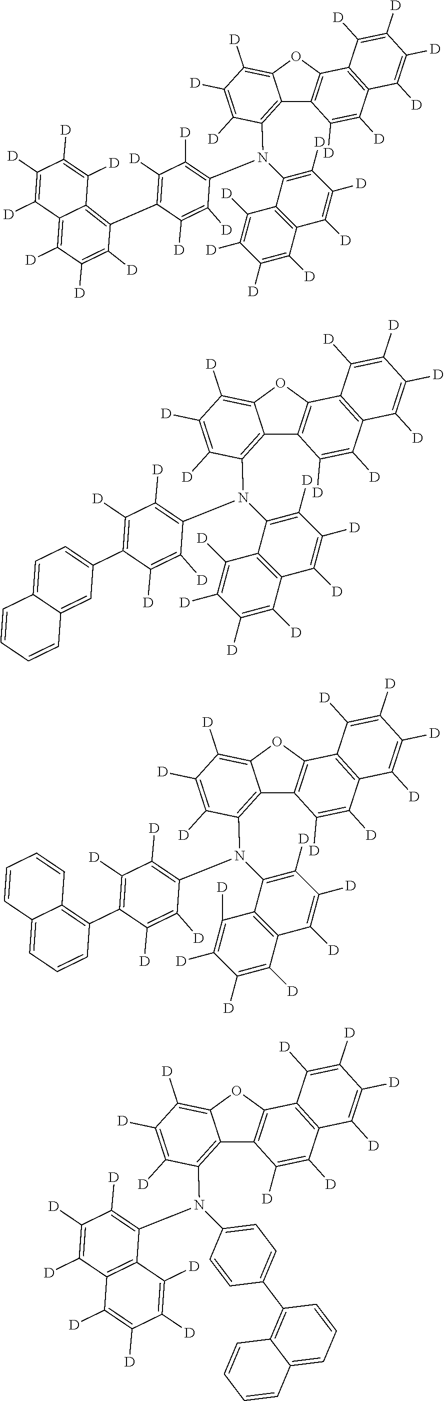

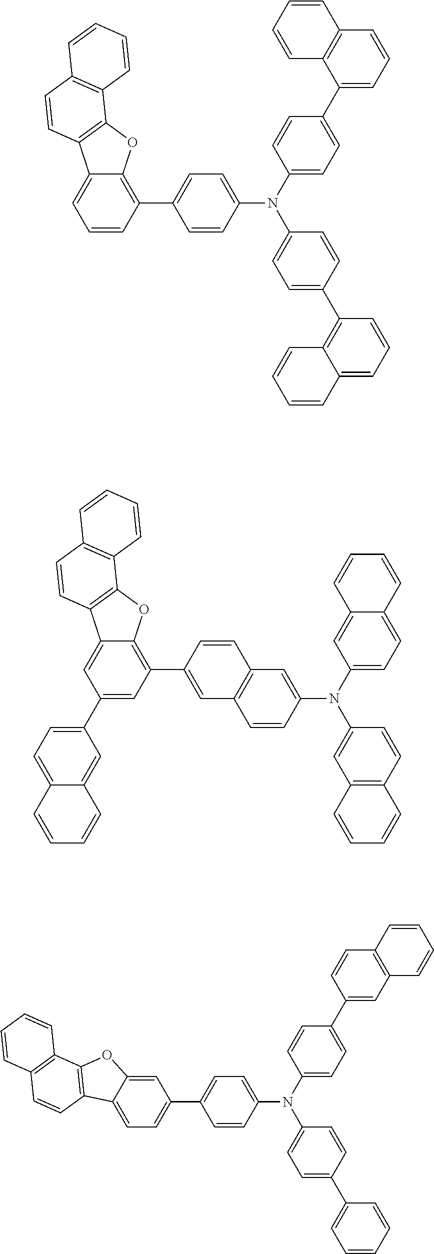 [SMILES: c1ccc2c(-c3ccc(N(c4ccc(-c5cccc6ccccc56)cc4)c4ccc(-c5cccc6c5oc5c7ccccc7ccc65)cc4)cc3)cccc2c1 ; c1ccc2cc(-c3cc(-c4ccc5cc(N(c6ccc7ccccc7c6)c6ccc7ccccc7c6)ccc5c4)c4oc5c6ccccc6ccc5c4c3)ccc2c1 ; c1ccc(-c2ccc(N(c3ccc(-c4ccc5ccccc5c4)cc3)c3ccc(-c4ccc5c(c4)oc4c6ccccc6ccc54)cc3)cc2)cc1]

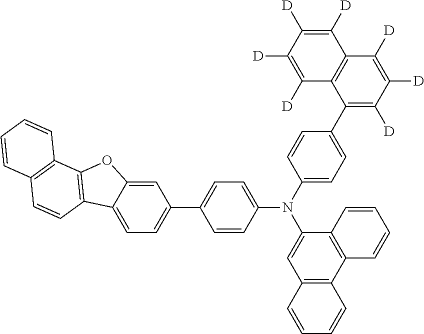 [SMILES: [2H]c1c([2H])c([2H])c2c(-c3ccc(N(c4ccc(-c5ccc6c(c5)oc5c7ccccc7ccc65)cc4)c4cc5ccccc5c5ccccc45)cc3)c([2H])c([2H])c([2H])c2c1[2H]]

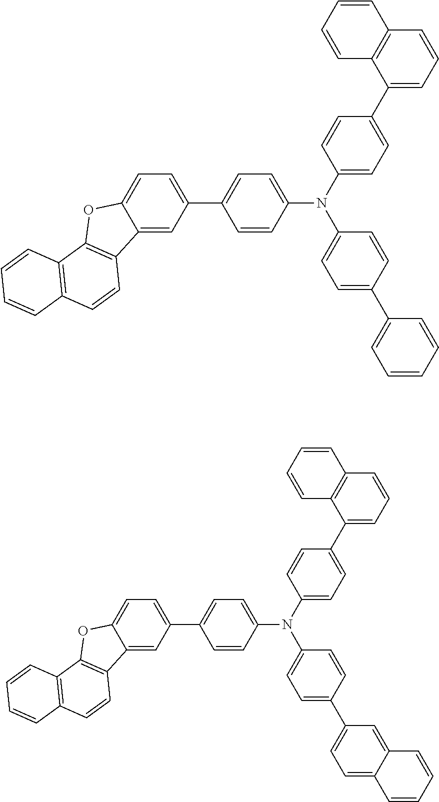 [SMILES: c1ccc(-c2ccc(N(c3ccc(-c4ccc5oc6c7ccccc7ccc6c5c4)cc3)c3ccc(-c4cccc5ccccc45)cc3)cc2)cc1 ; c1ccc2cc(-c3ccc(N(c4ccc(-c5ccc6oc7c8ccccc8ccc7c6c5)cc4)c4ccc(-c5cccc6ccccc56)cc4)cc3)ccc2c1]

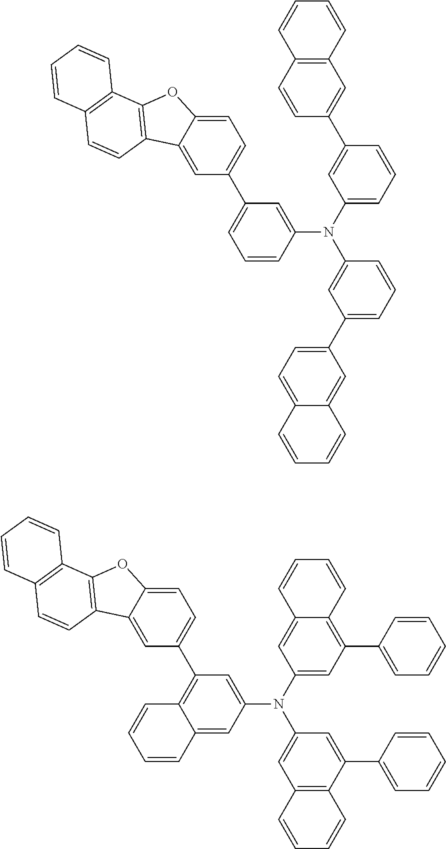 [SMILES: c1cc(-c2ccc3ccccc3c2)cc(N(c2cccc(-c3ccc4ccccc4c3)c2)c2cccc(-c3ccc4oc5c6ccccc6ccc5c4c3)c2)c1 ; c1ccc(-c2cc(N(c3cc(-c4ccccc4)c4ccccc4c3)c3cc(-c4ccc5oc6c7ccccc7ccc6c5c4)c4ccccc4c3)cc3ccccc23)cc1]

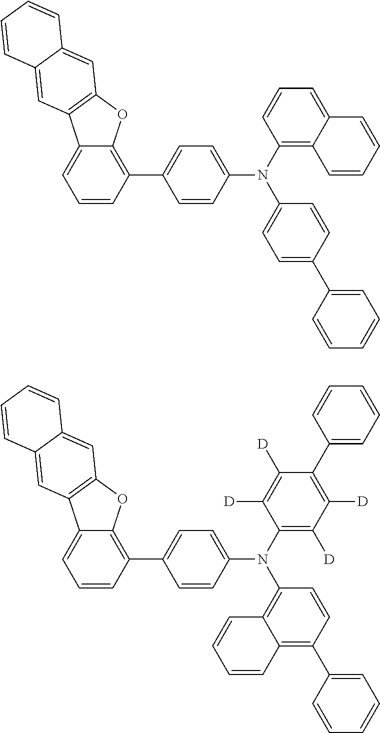

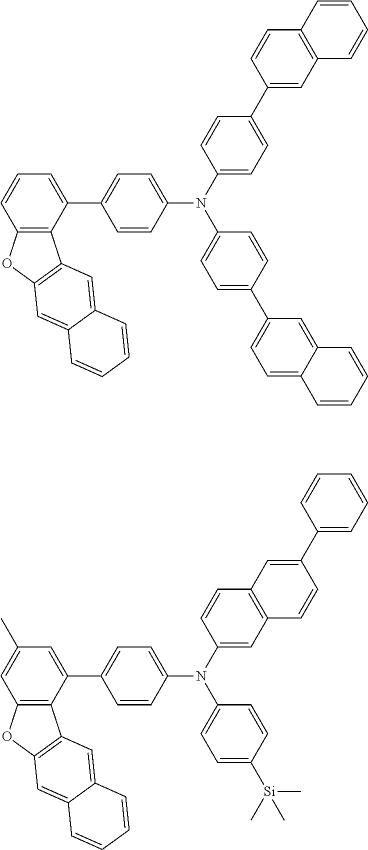 [SMILES: c1ccc2cc(-c3ccc(N(c4ccc(-c5ccc6ccccc6c5)cc4)c4ccc(-c5cccc6oc7cc8ccccc8cc7c56)cc4)cc3)ccc2c1 ; Cc1cc(-c2ccc(N(c3ccc([Si](C)(C)C)cc3)c3ccc4cc(-c5ccccc5)ccc4c3)cc2)c2c(c1)oc1cc3ccccc3cc12]

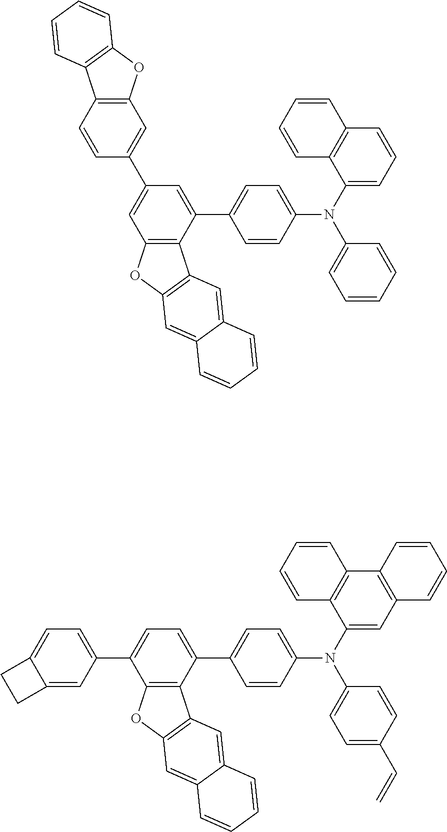 [SMILES: c1ccc(N(c2ccc(-c3cc(-c4ccc5c(c4)oc4ccccc45)cc4oc5cc6ccccc6cc5c34)cc2)c2cccc3ccccc23)cc1 ; C=Cc1ccc(N(c2ccc(-c3ccc(-c4ccc5c(c4)CC5)c4oc5cc6ccccc6cc5c34)cc2)c2cc3ccccc3c3ccccc23)cc1]

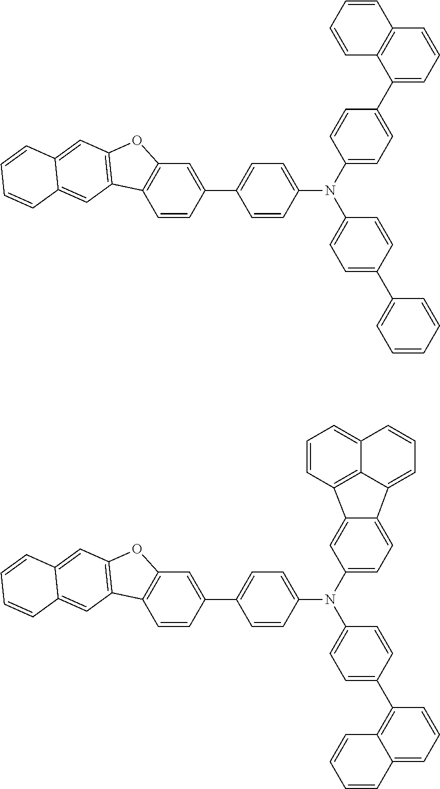 [SMILES: c1ccc(-c2ccc(N(c3ccc(-c4ccc5c(c4)oc4cc6ccccc6cc45)cc3)c3ccc(-c4cccc5ccccc45)cc3)cc2)cc1 ; c1ccc2cc3c(cc2c1)oc1cc(-c2ccc(N(c4ccc(-c5cccc6ccccc56)cc4)c4ccc5c(c4)-c4cccc6cccc-5c46)cc2)ccc13]

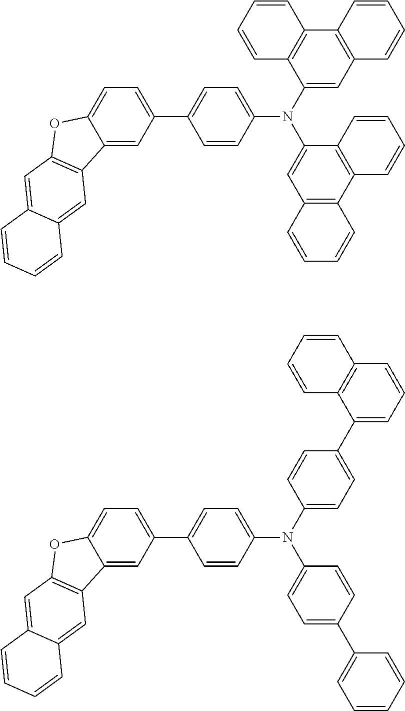 [SMILES: c1ccc2cc3c(cc2c1)oc1ccc(-c2ccc(N(c4cc5ccccc5c5ccccc45)c4cc5ccccc5c5ccccc45)cc2)cc13 ; c1ccc(-c2ccc(N(c3ccc(-c4ccc5oc6cc7ccccc7cc6c5c4)cc3)c3ccc(-c4cccc5ccccc45)cc3)cc2)cc1]

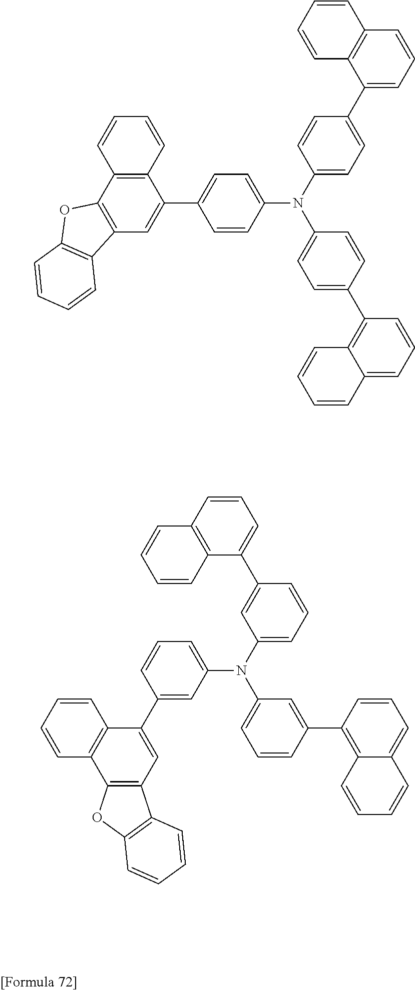 [SMILES: c1ccc2c(-c3ccc(N(c4ccc(-c5cccc6ccccc56)cc4)c4ccc(-c5cc6c7ccccc7oc6c6ccccc56)cc4)cc3)cccc2c1 ; c1cc(-c2cccc3ccccc23)cc(N(c2cccc(-c3cccc4ccccc34)c2)c2cccc(-c3cc4c5ccccc5oc4c4ccccc34)c2)c1]

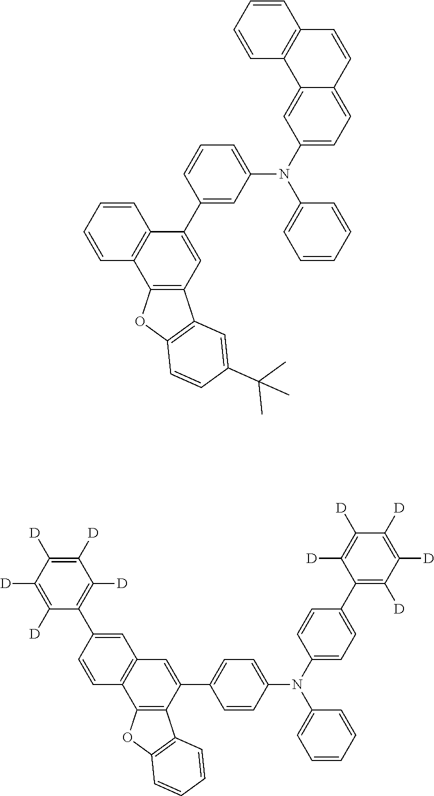 [SMILES: CC(C)(C)c1ccc2oc3c4ccccc4c(-c4cccc(N(c5ccccc5)c5ccc6ccc7ccccc7c6c5)c4)cc3c2c1 ; [2H]c1c([2H])c([2H])c(-c2ccc(N(c3ccccc3)c3ccc(-c4cc5cc(-c6c([2H])c([2H])c([2H])c([2H])c6[2H])ccc5c5oc6ccccc6c45)cc3)cc2)c([2H])c1[2H]]

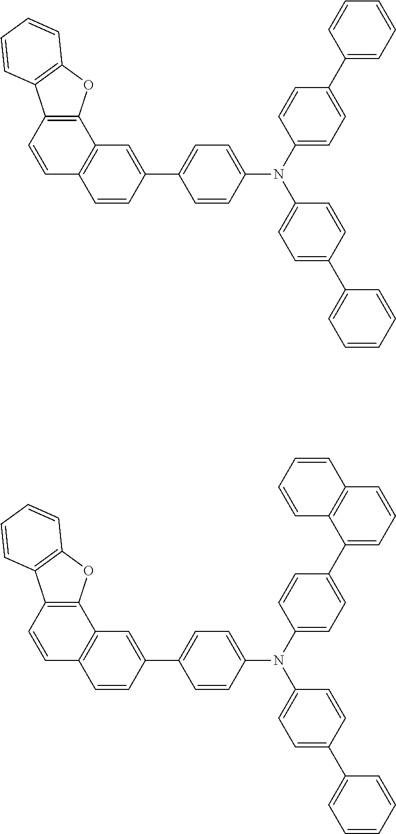 [SMILES: c1ccc(-c2ccc(N(c3ccc(-c4ccccc4)cc3)c3ccc(-c4ccc5ccc6c7ccccc7oc6c5c4)cc3)cc2)cc1 ; c1ccc(-c2ccc(N(c3ccc(-c4ccc5ccc6c7ccccc7oc6c5c4)cc3)c3ccc(-c4cccc5ccccc45)cc3)cc2)cc1]

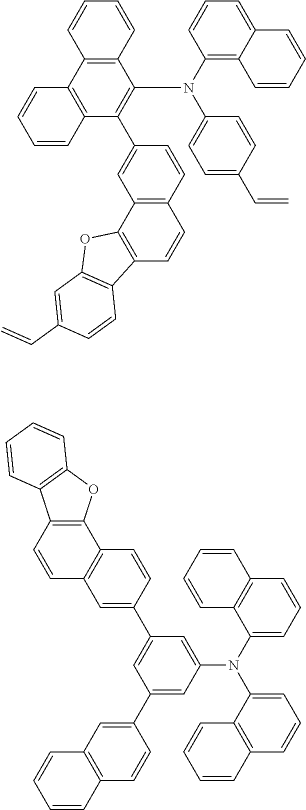 [SMILES: C=Cc1ccc(N(c2cccc3ccccc23)c2c(-c3ccc4ccc5c6ccc(C=C)cc6oc5c4c3)c3ccccc3c3ccccc23)cc1 ; c1ccc2cc(-c3cc(-c4ccc5c(ccc6c7ccccc7oc56)c4)cc(N(c4cccc5ccccc45)c4cccc5ccccc45)c3)ccc2c1]

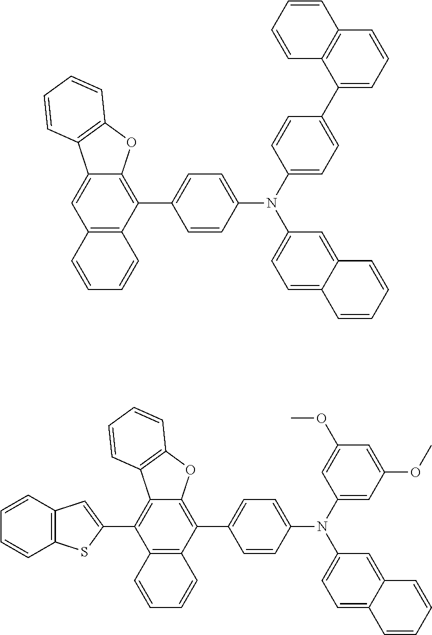 [SMILES: c1ccc2cc(N(c3ccc(-c4cccc5ccccc45)cc3)c3ccc(-c4c5ccccc5cc5c4oc4ccccc45)cc3)ccc2c1 ; COc1cc(OC)cc(N(c2ccc(-c3c4ccccc4c(-c4cc5ccccc5s4)c4c3oc3ccccc34)cc2)c2ccc3ccccc3c2)c1]

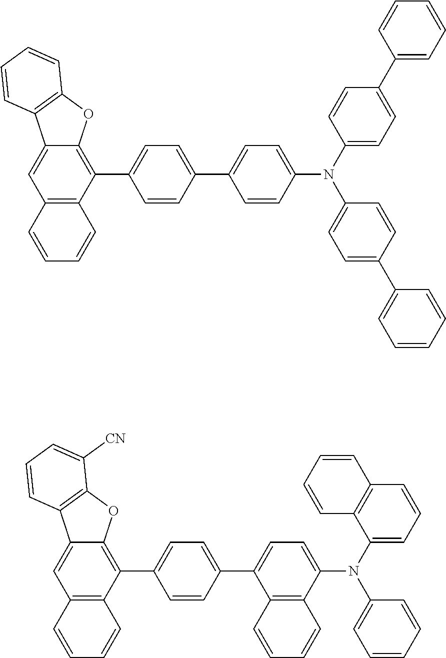 [SMILES: c1ccc(-c2ccc(N(c3ccc(-c4ccccc4)cc3)c3ccc(-c4ccc(-c5c6ccccc6cc6c5oc5ccccc56)cc4)cc3)cc2)cc1 ; N#Cc1cccc2c1oc1c(-c3ccc(-c4ccc(N(c5ccccc5)c5cccc6ccccc56)c5ccccc45)cc3)c3ccccc3cc12]

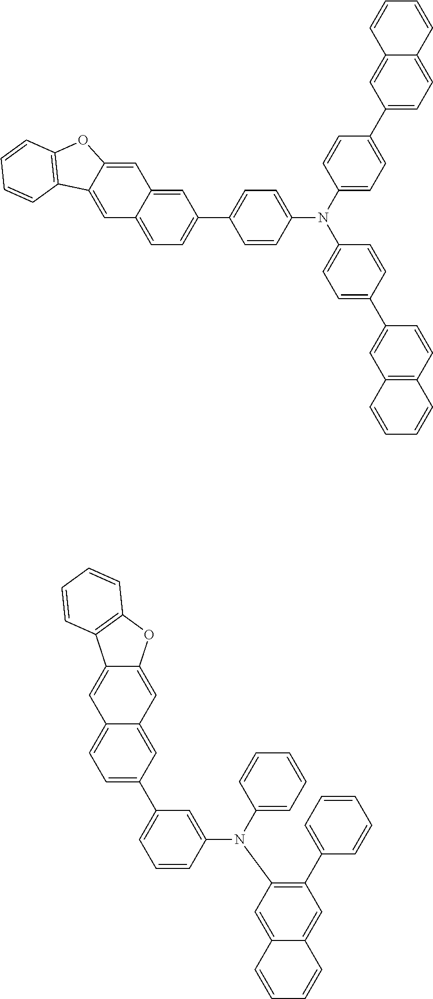 [SMILES: c1ccc2cc(-c3ccc(N(c4ccc(-c5ccc6ccccc6c5)cc4)c4ccc(-c5ccc6cc7c(cc6c5)oc5ccccc57)cc4)cc3)ccc2c1 ; c1ccc(-c2cc3ccccc3cc2N(c2ccccc2)c2cccc(-c3ccc4cc5c(cc4c3)oc3ccccc35)c2)cc1]

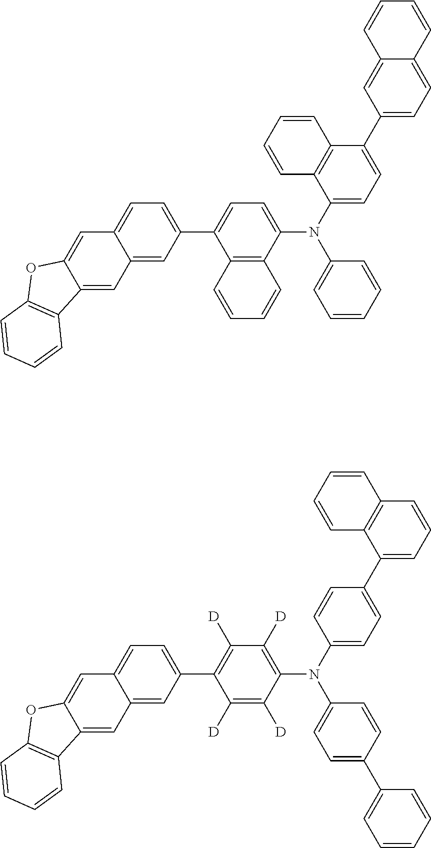 [SMILES: c1ccc(N(c2ccc(-c3ccc4ccccc4c3)c3ccccc23)c2ccc(-c3ccc4cc5oc6ccccc6c5cc4c3)c3ccccc23)cc1 ; [2H]c1c([2H])c(N(c2ccc(-c3ccccc3)cc2)c2ccc(-c3cccc4ccccc34)cc2)c([2H])c([2H])c1-c1ccc2cc3oc4ccccc4c3cc2c1]

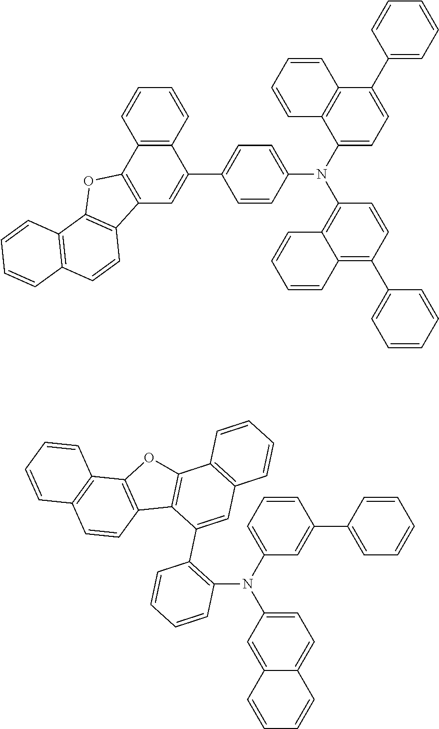 [SMILES: c1ccc(-c2ccc(N(c3ccc(-c4cc5c6ccc7ccccc7c6oc5c5ccccc45)cc3)c3ccc(-c4ccccc4)c4ccccc34)c3ccccc23)cc1 ; c1ccc(-c2cccc(N(c3ccc4ccccc4c3)c3ccccc3-c3cc4ccccc4c4oc5c6ccccc6ccc5c34)c2)cc1]

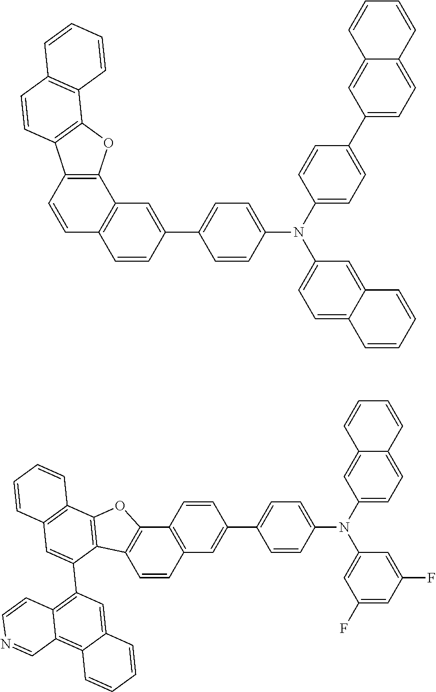 [SMILES: c1ccc2cc(-c3ccc(N(c4ccc(-c5ccc6ccc7c8ccc9ccccc9c8oc7c6c5)cc4)c4ccc5ccccc5c4)cc3)ccc2c1 ; Fc1cc(F)cc(N(c2ccc(-c3ccc4c(ccc5c4oc4c6ccccc6cc(-c6cc7ccccc7c7cnccc67)c54)c3)cc2)c2ccc3ccccc3c2)c1]

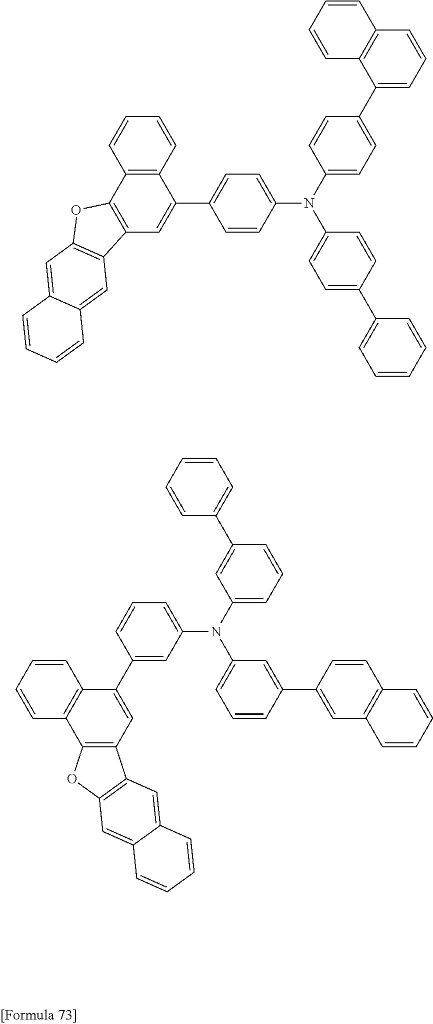 [SMILES: c1ccc(-c2ccc(N(c3ccc(-c4cccc5ccccc45)cc3)c3ccc(-c4cc5c6cc7ccccc7cc6oc5c5ccccc45)cc3)cc2)cc1 ; c1ccc(-c2cccc(N(c3cccc(-c4ccc5ccccc5c4)c3)c3cccc(-c4cc5c6cc7ccccc7cc6oc5c5ccccc45)c3)c2)cc1]

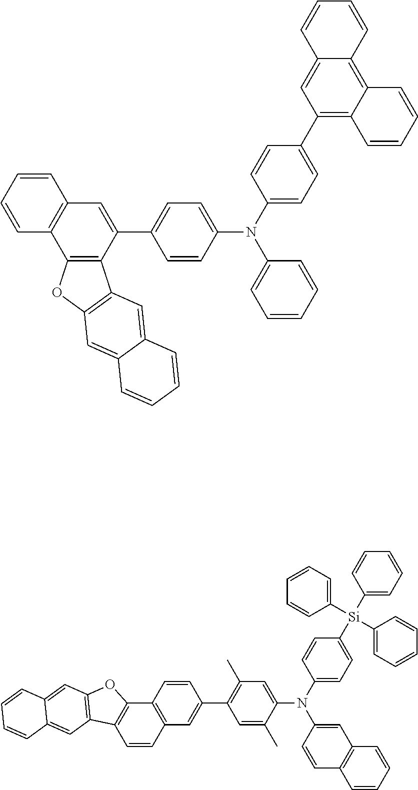 [SMILES: c1ccc(N(c2ccc(-c3cc4ccccc4c4ccccc34)cc2)c2ccc(-c3cc4ccccc4c4oc5cc6ccccc6cc5c34)cc2)cc1 ; Cc1cc(N(c2ccc([Si](c3ccccc3)(c3ccccc3)c3ccccc3)cc2)c2ccc3ccccc3c2)c(C)cc1-c1ccc2c(ccc3c4cc5ccccc5cc4oc23)c1]

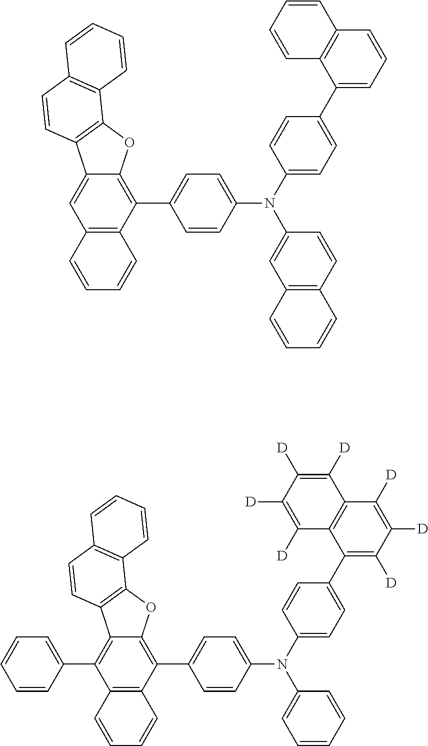 [SMILES: c1ccc2cc(N(c3ccc(-c4cccc5ccccc45)cc3)c3ccc(-c4c5ccccc5cc5c4oc4c6ccccc6ccc54)cc3)ccc2c1 ; [2H]c1c([2H])c([2H])c2c(-c3ccc(N(c4ccccc4)c4ccc(-c5c6ccccc6c(-c6ccccc6)c6c5oc5c7ccccc7ccc56)cc4)cc3)c([2H])c([2H])c([2H])c2c1[2H]]

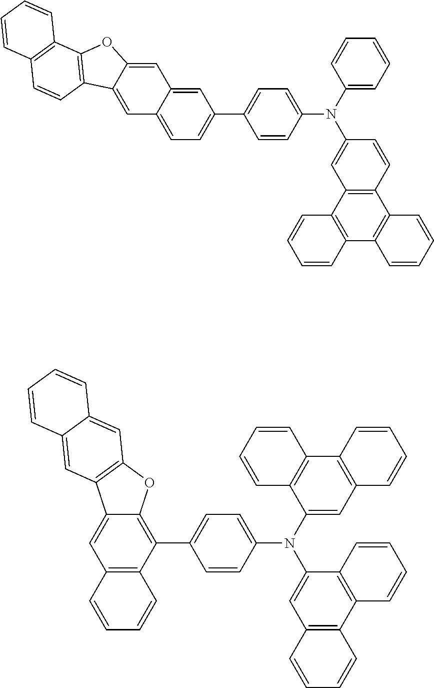 [SMILES: c1ccc(N(c2ccc(-c3ccc4cc5c(cc4c3)oc3c4ccccc4ccc53)cc2)c2ccc3c4ccccc4c4ccccc4c3c2)cc1 ; c1ccc2cc3c(cc2c1)oc1c(-c2ccc(N(c4cc5ccccc5c5ccccc45)c4cc5ccccc5c5ccccc45)cc2)c2ccccc2cc13]

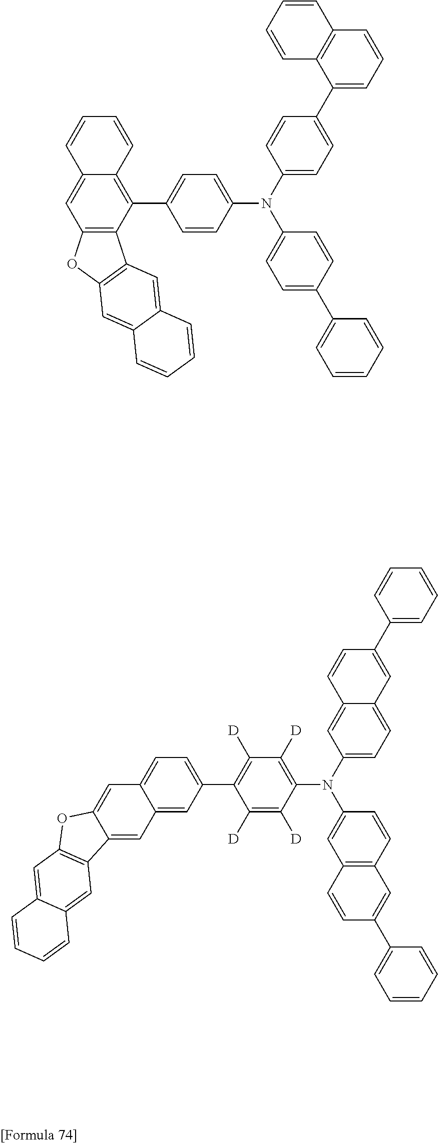 [SMILES: c1ccc(-c2ccc(N(c3ccc(-c4cccc5ccccc45)cc3)c3ccc(-c4c5ccccc5cc5oc6cc7ccccc7cc6c45)cc3)cc2)cc1 ; [2H]c1c([2H])c(N(c2ccc3cc(-c4ccccc4)ccc3c2)c2ccc3cc(-c4ccccc4)ccc3c2)c([2H])c([2H])c1-c1ccc2cc3oc4cc5ccccc5cc4c3cc2c1]

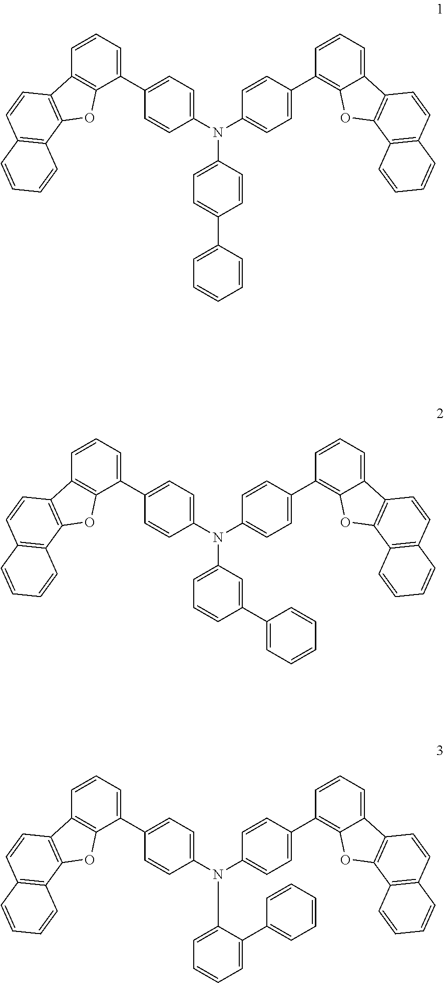 [SMILES: c1ccc(-c2ccc(N(c3ccc(-c4cccc5c4oc4c6ccccc6ccc54)cc3)c3ccc(-c4cccc5c4oc4c6ccccc6ccc54)cc3)cc2)cc1 ; c1ccc(-c2cccc(N(c3ccc(-c4cccc5c4oc4c6ccccc6ccc54)cc3)c3ccc(-c4cccc5c4oc4c6ccccc6ccc54)cc3)c2)cc1 ; c1ccc(-c2ccccc2N(c2ccc(-c3cccc4c3oc3c5ccccc5ccc43)cc2)c2ccc(-c3cccc4c3oc3c5ccccc5ccc43)cc2)cc1]

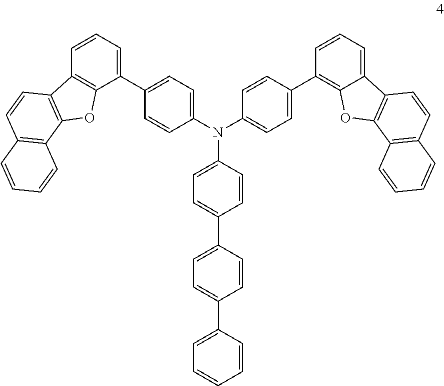 [SMILES: c1ccc(-c2ccc(-c3ccc(N(c4ccc(-c5cccc6c5oc5c7ccccc7ccc65)cc4)c4ccc(-c5cccc6c5oc5c7ccccc7ccc65)cc4)cc3)cc2)cc1]

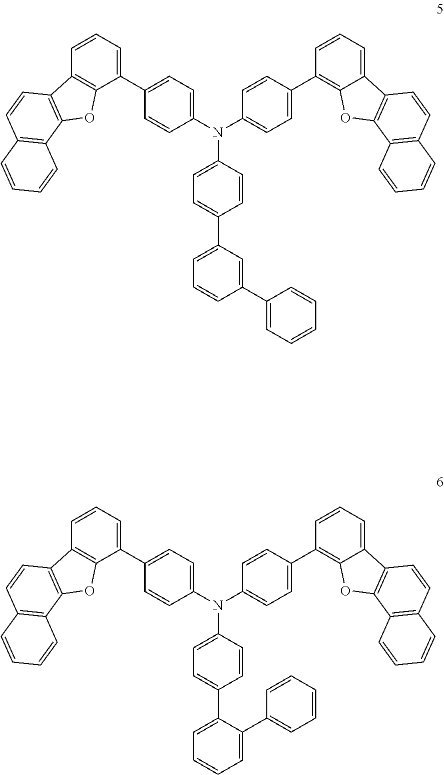 [SMILES: c1ccc(-c2cccc(-c3ccc(N(c4ccc(-c5cccc6c5oc5c7ccccc7ccc65)cc4)c4ccc(-c5cccc6c5oc5c7ccccc7ccc65)cc4)cc3)c2)cc1 ; c1ccc(-c2ccccc2-c2ccc(N(c3ccc(-c4cccc5c4oc4c6ccccc6ccc54)cc3)c3ccc(-c4cccc5c4oc4c6ccccc6ccc54)cc3)cc2)cc1]

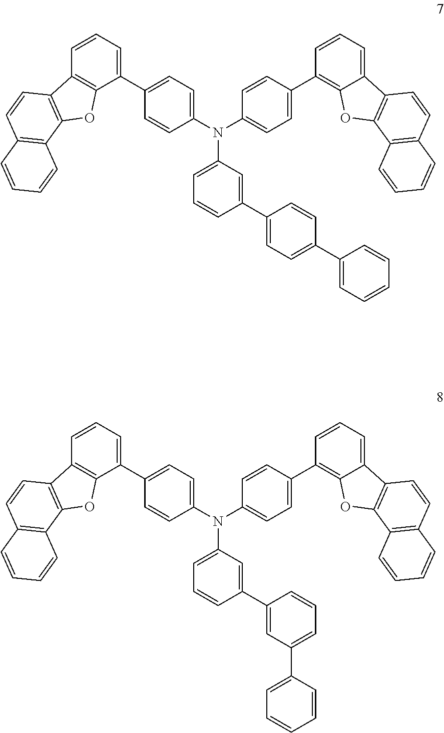 [SMILES: c1ccc(-c2ccc(-c3cccc(N(c4ccc(-c5cccc6c5oc5c7ccccc7ccc65)cc4)c4ccc(-c5cccc6c5oc5c7ccccc7ccc65)cc4)c3)cc2)cc1 ; c1ccc(-c2cccc(-c3cccc(N(c4ccc(-c5cccc6c5oc5c7ccccc7ccc65)cc4)c4ccc(-c5cccc6c5oc5c7ccccc7ccc65)cc4)c3)c2)cc1]

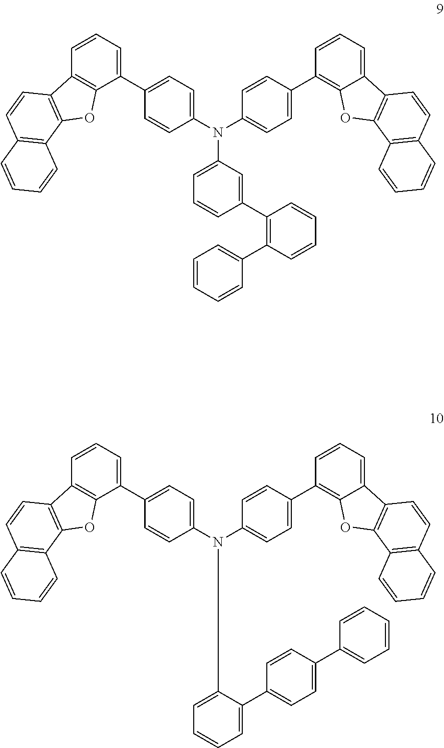 [SMILES: c1ccc(-c2ccccc2-c2cccc(N(c3ccc(-c4cccc5c4oc4c6ccccc6ccc54)cc3)c3ccc(-c4cccc5c4oc4c6ccccc6ccc54)cc3)c2)cc1 ; c1ccc(-c2ccc(-c3ccccc3N(c3ccc(-c4cccc5c4oc4c6ccccc6ccc54)cc3)c3ccc(-c4cccc5c4oc4c6ccccc6ccc54)cc3)cc2)cc1]

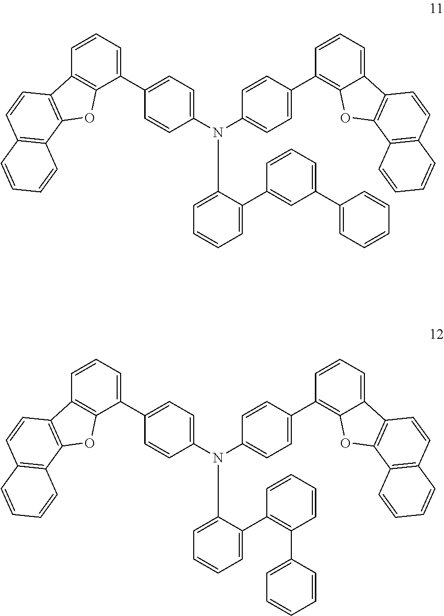 [SMILES: c1ccc(-c2cccc(-c3ccccc3N(c3ccc(-c4cccc5c4oc4c6ccccc6ccc54)cc3)c3ccc(-c4cccc5c4oc4c6ccccc6ccc54)cc3)c2)cc1 ; c1ccc(-c2ccccc2-c2ccccc2N(c2ccc(-c3cccc4c3oc3c5ccccc5ccc43)cc2)c2ccc(-c3cccc4c3oc3c5ccccc5ccc43)cc2)cc1]

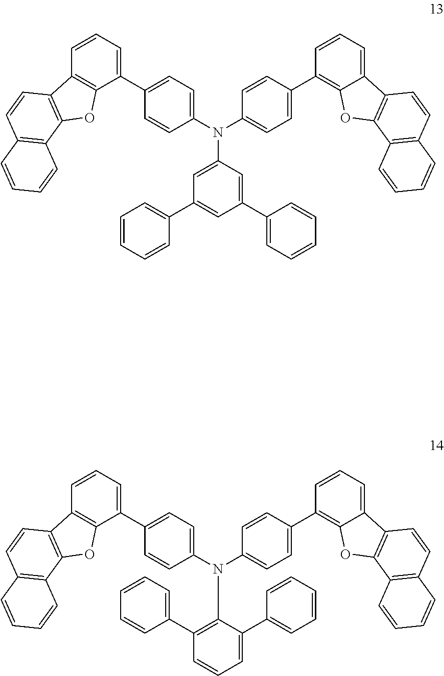 [SMILES: c1ccc(-c2cc(-c3ccccc3)cc(N(c3ccc(-c4cccc5c4oc4c6ccccc6ccc54)cc3)c3ccc(-c4cccc5c4oc4c6ccccc6ccc54)cc3)c2)cc1 ; c1ccc(-c2cccc(-c3ccccc3)c2N(c2ccc(-c3cccc4c3oc3c5ccccc5ccc43)cc2)c2ccc(-c3cccc4c3oc3c5ccccc5ccc43)cc2)cc1]

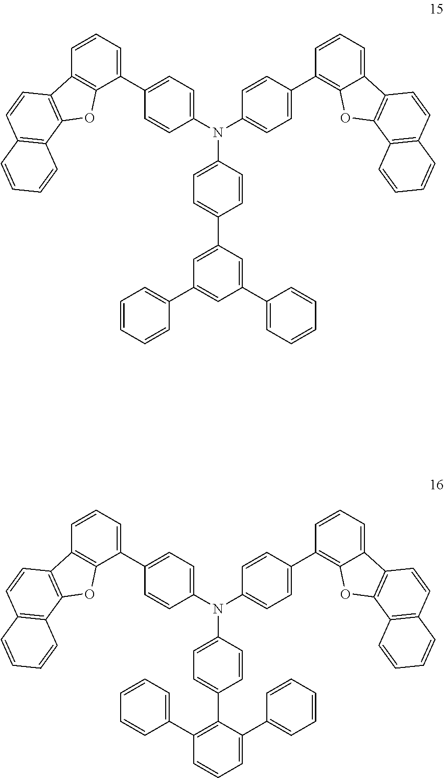 [SMILES: c1ccc(-c2cc(-c3ccccc3)cc(-c3ccc(N(c4ccc(-c5cccc6c5oc5c7ccccc7ccc65)cc4)c4ccc(-c5cccc6c5oc5c7ccccc7ccc65)cc4)cc3)c2)cc1 ; c1ccc(-c2cccc(-c3ccccc3)c2-c2ccc(N(c3ccc(-c4cccc5c4oc4c6ccccc6ccc54)cc3)c3ccc(-c4cccc5c4oc4c6ccccc6ccc54)cc3)cc2)cc1]

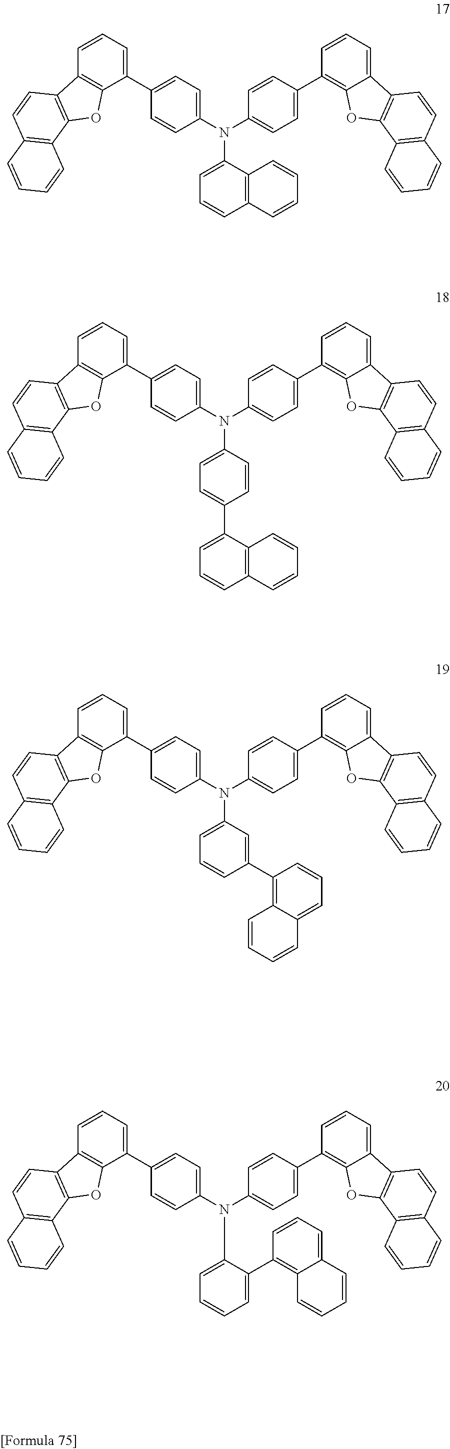 [SMILES: c1ccc2c(N(c3ccc(-c4cccc5c4oc4c6ccccc6ccc54)cc3)c3ccc(-c4cccc5c4oc4c6ccccc6ccc54)cc3)cccc2c1 ; c1ccc2c(-c3ccc(N(c4ccc(-c5cccc6c5oc5c7ccccc7ccc65)cc4)c4ccc(-c5cccc6c5oc5c7ccccc7ccc65)cc4)cc3)cccc2c1 ; c1cc(-c2cccc3ccccc23)cc(N(c2ccc(-c3cccc4c3oc3c5ccccc5ccc43)cc2)c2ccc(-c3cccc4c3oc3c5ccccc5ccc43)cc2)c1 ; c1ccc(N(c2ccc(-c3cccc4c3oc3c5ccccc5ccc43)cc2)c2ccc(-c3cccc4c3oc3c5ccccc5ccc43)cc2)c(-c2cccc3ccccc23)c1]

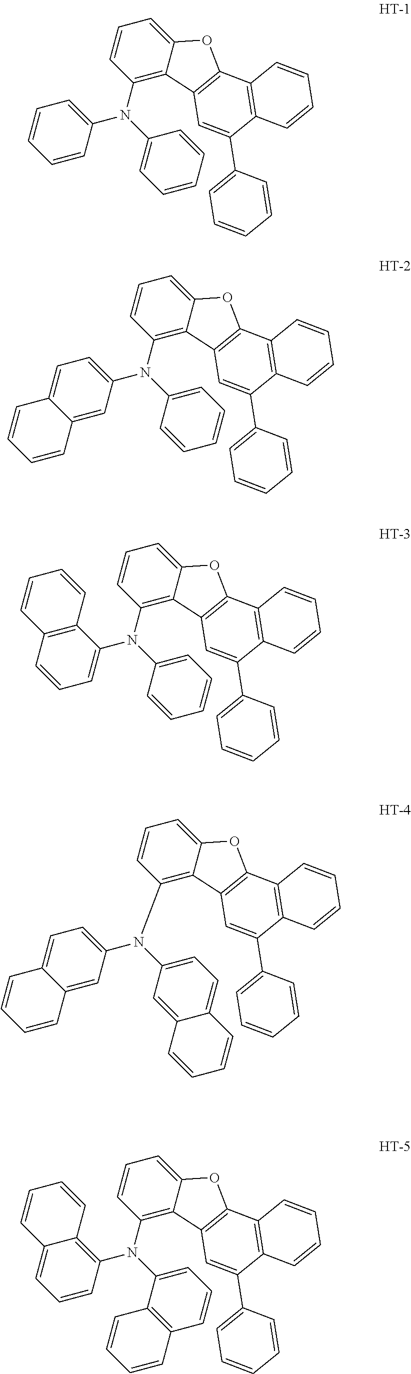 [SMILES: c1ccc(-c2cc3c(oc4cccc(N(c5ccccc5)c5ccccc5)c43)c3ccccc23)cc1 ; c1ccc(-c2cc3c(oc4cccc(N(c5ccccc5)c5ccc6ccccc6c5)c43)c3ccccc23)cc1 ; c1ccc(-c2cc3c(oc4cccc(N(c5ccccc5)c5cccc6ccccc56)c43)c3ccccc23)cc1 ; c1ccc(-c2cc3c(oc4cccc(N(c5ccc6ccccc6c5)c5ccc6ccccc6c5)c43)c3ccccc23)cc1 ; c1ccc(-c2cc3c(oc4cccc(N(c5cccc6ccccc56)c5cccc6ccccc56)c43)c3ccccc23)cc1]

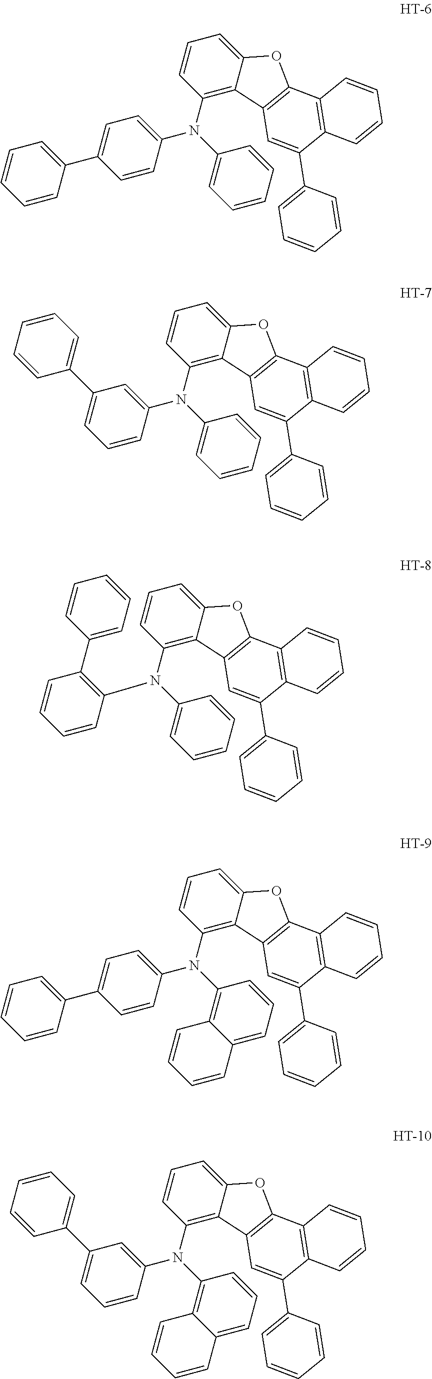 [SMILES: c1ccc(-c2ccc(N(c3ccccc3)c3cccc4oc5c6ccccc6c(-c6ccccc6)cc5c34)cc2)cc1 ; c1ccc(-c2cccc(N(c3ccccc3)c3cccc4oc5c6ccccc6c(-c6ccccc6)cc5c34)c2)cc1 ; c1ccc(-c2ccccc2N(c2ccccc2)c2cccc3oc4c5ccccc5c(-c5ccccc5)cc4c23)cc1 ; c1ccc(-c2ccc(N(c3cccc4ccccc34)c3cccc4oc5c6ccccc6c(-c6ccccc6)cc5c34)cc2)cc1 ; c1ccc(-c2cccc(N(c3cccc4ccccc34)c3cccc4oc5c6ccccc6c(-c6ccccc6)cc5c34)c2)cc1]

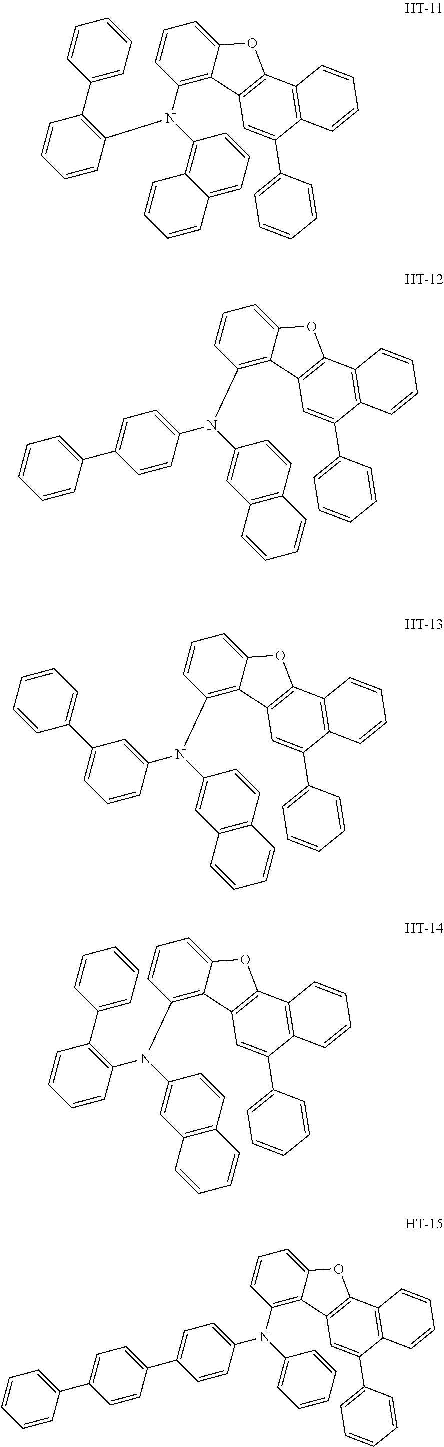 [SMILES: c1ccc(-c2ccccc2N(c2cccc3ccccc23)c2cccc3oc4c5ccccc5c(-c5ccccc5)cc4c23)cc1 ; c1ccc(-c2ccc(N(c3ccc4ccccc4c3)c3cccc4oc5c6ccccc6c(-c6ccccc6)cc5c34)cc2)cc1 ; c1ccc(-c2cccc(N(c3ccc4ccccc4c3)c3cccc4oc5c6ccccc6c(-c6ccccc6)cc5c34)c2)cc1 ; c1ccc(-c2ccccc2N(c2ccc3ccccc3c2)c2cccc3oc4c5ccccc5c(-c5ccccc5)cc4c23)cc1 ; c1ccc(-c2ccc(-c3ccc(N(c4ccccc4)c4cccc5oc6c7ccccc7c(-c7ccccc7)cc6c45)cc3)cc2)cc1]

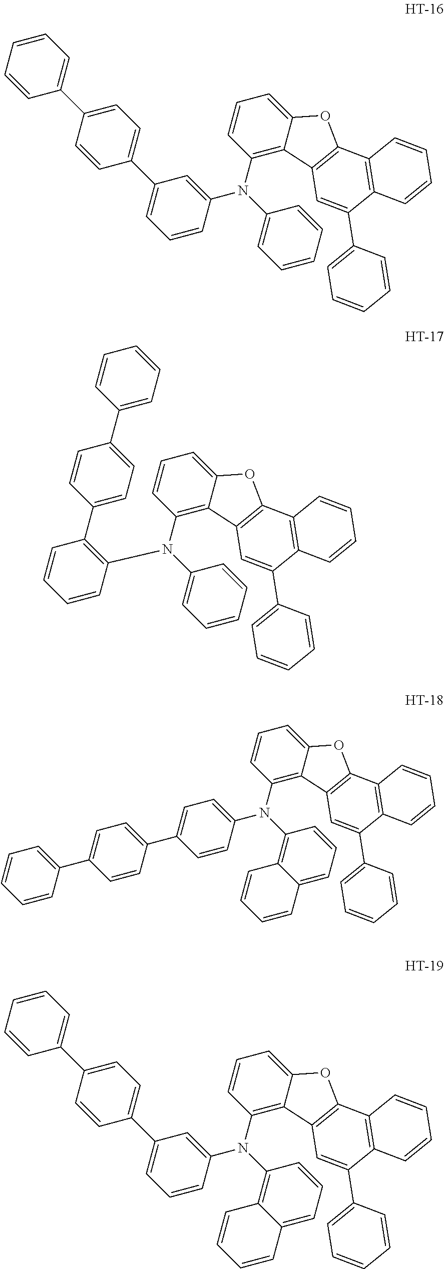 [SMILES: c1ccc(-c2ccc(-c3cccc(N(c4ccccc4)c4cccc5oc6c7ccccc7c(-c7ccccc7)cc6c45)c3)cc2)cc1 ; c1ccc(-c2ccc(-c3ccccc3N(c3ccccc3)c3cccc4oc5c6ccccc6c(-c6ccccc6)cc5c34)cc2)cc1 ; c1ccc(-c2ccc(-c3ccc(N(c4cccc5ccccc45)c4cccc5oc6c7ccccc7c(-c7ccccc7)cc6c45)cc3)cc2)cc1 ; c1ccc(-c2ccc(-c3cccc(N(c4cccc5ccccc45)c4cccc5oc6c7ccccc7c(-c7ccccc7)cc6c45)c3)cc2)cc1]

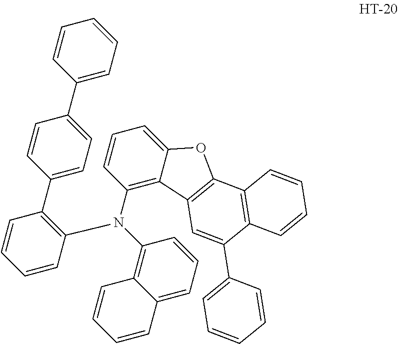 [SMILES: c1ccc(-c2ccc(-c3ccccc3N(c3cccc4ccccc34)c3cccc4oc5c6ccccc6c(-c6ccccc6)cc5c34)cc2)cc1]

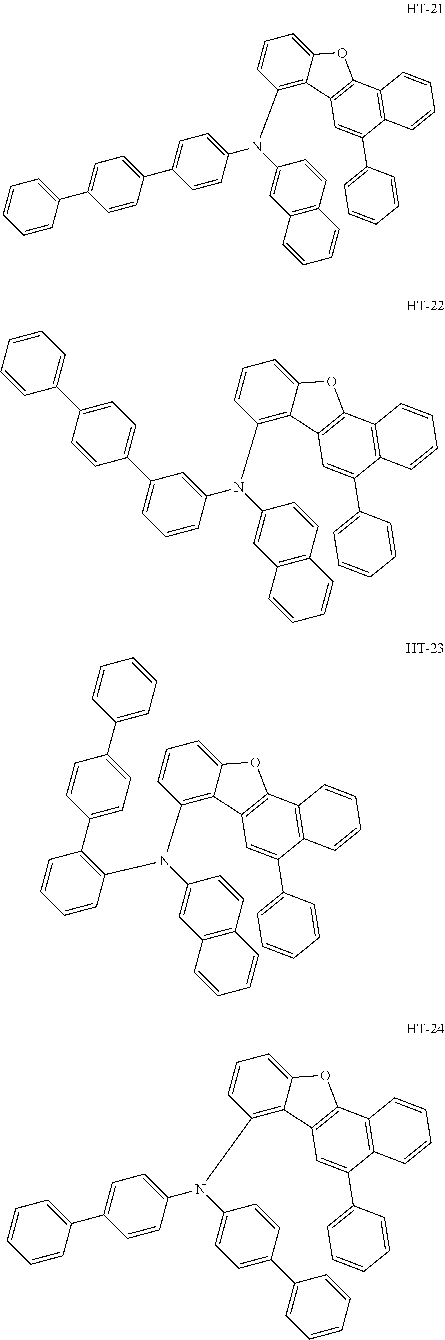 [SMILES: c1ccc(-c2ccc(-c3ccc(N(c4ccc5ccccc5c4)c4cccc5oc6c7ccccc7c(-c7ccccc7)cc6c45)cc3)cc2)cc1 ; c1ccc(-c2ccc(-c3cccc(N(c4ccc5ccccc5c4)c4cccc5oc6c7ccccc7c(-c7ccccc7)cc6c45)c3)cc2)cc1 ; c1ccc(-c2ccc(-c3ccccc3N(c3ccc4ccccc4c3)c3cccc4oc5c6ccccc6c(-c6ccccc6)cc5c34)cc2)cc1 ; c1ccc(-c2ccc(N(c3ccc(-c4ccccc4)cc3)c3cccc4oc5c6ccccc6c(-c6ccccc6)cc5c34)cc2)cc1]

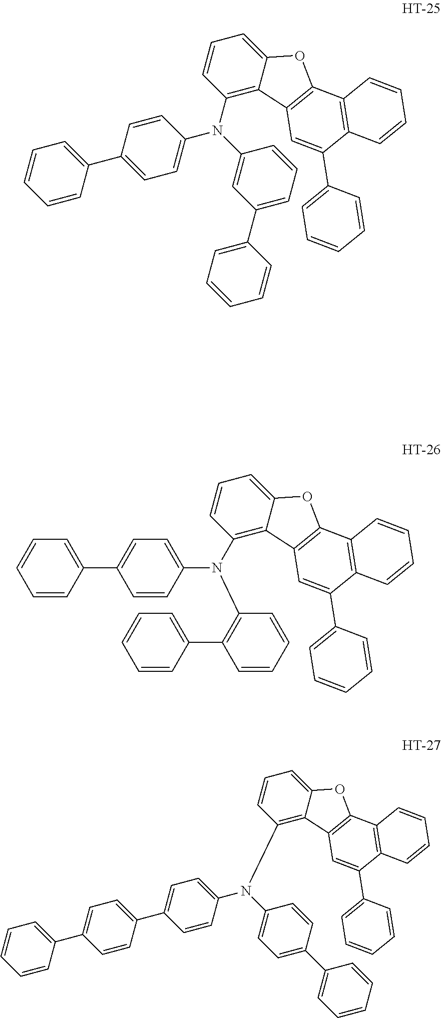 [SMILES: c1ccc(-c2ccc(N(c3cccc(-c4ccccc4)c3)c3cccc4oc5c6ccccc6c(-c6ccccc6)cc5c34)cc2)cc1 ; c1ccc(-c2ccc(N(c3ccccc3-c3ccccc3)c3cccc4oc5c6ccccc6c(-c6ccccc6)cc5c34)cc2)cc1 ; c1ccc(-c2ccc(-c3ccc(N(c4ccc(-c5ccccc5)cc4)c4cccc5oc6c7ccccc7c(-c7ccccc7)cc6c45)cc3)cc2)cc1]

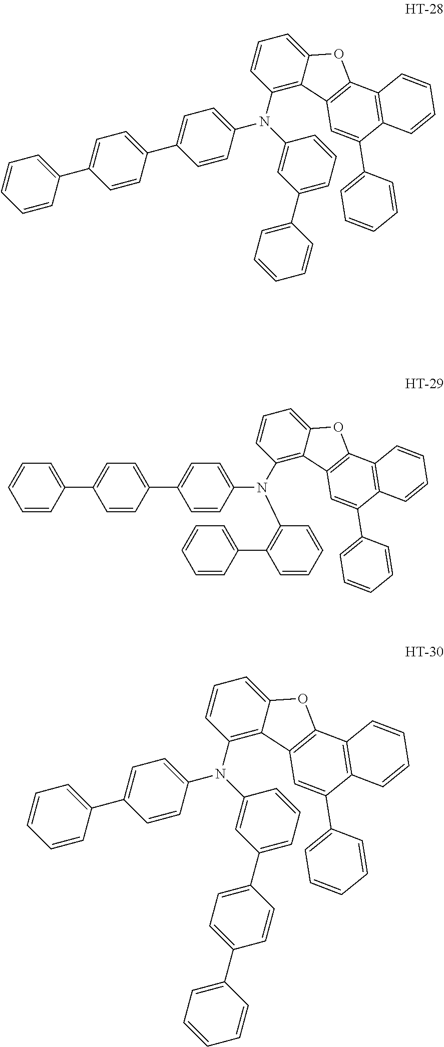 [SMILES: c1ccc(-c2ccc(-c3ccc(N(c4cccc(-c5ccccc5)c4)c4cccc5oc6c7ccccc7c(-c7ccccc7)cc6c45)cc3)cc2)cc1 ; c1ccc(-c2ccc(-c3ccc(N(c4ccccc4-c4ccccc4)c4cccc5oc6c7ccccc7c(-c7ccccc7)cc6c45)cc3)cc2)cc1 ; c1ccc(-c2ccc(-c3cccc(N(c4ccc(-c5ccccc5)cc4)c4cccc5oc6c7ccccc7c(-c7ccccc7)cc6c45)c3)cc2)cc1]

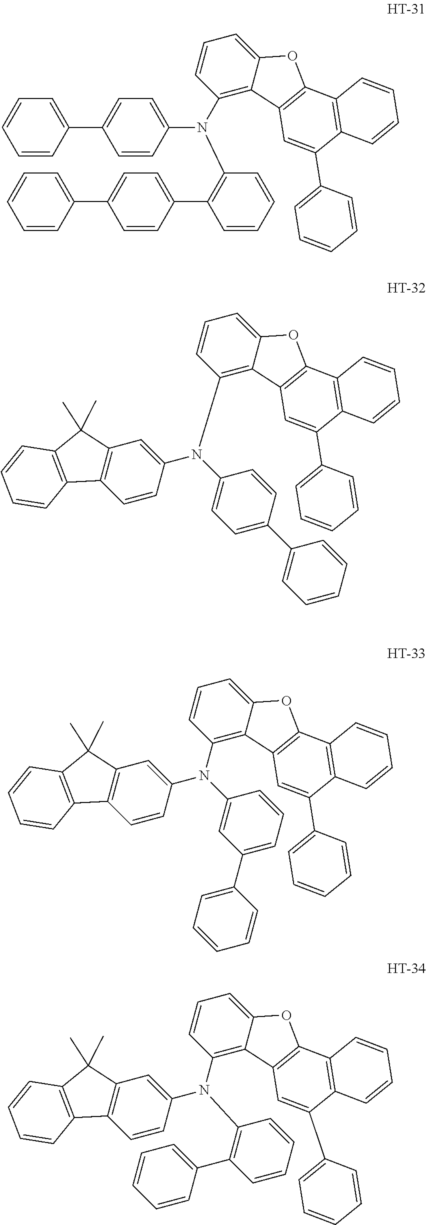

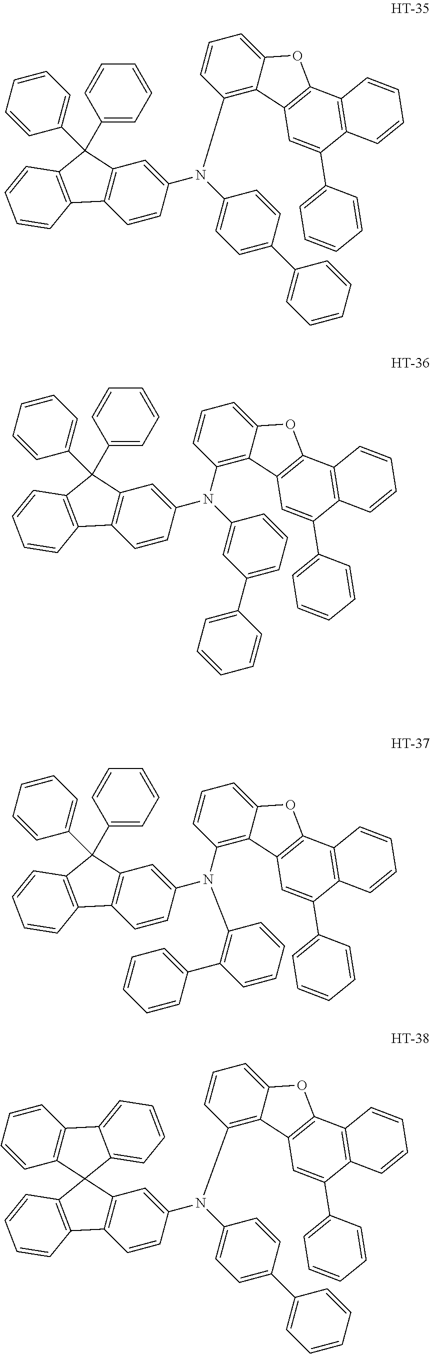 [SMILES: c1ccc(-c2ccc(N(c3ccc4c(c3)C(c3ccccc3)(c3ccccc3)c3ccccc3-4)c3cccc4oc5c6ccccc6c(-c6ccccc6)cc5c34)cc2)cc1 ; c1ccc(-c2cccc(N(c3ccc4c(c3)C(c3ccccc3)(c3ccccc3)c3ccccc3-4)c3cccc4oc5c6ccccc6c(-c6ccccc6)cc5c34)c2)cc1 ; c1ccc(-c2ccccc2N(c2ccc3c(c2)C(c2ccccc2)(c2ccccc2)c2ccccc2-3)c2cccc3oc4c5ccccc5c(-c5ccccc5)cc4c23)cc1 ; c1ccc(-c2ccc(N(c3ccc4c(c3)C3(c5ccccc5-c5ccccc53)c3ccccc3-4)c3cccc4oc5c6ccccc6c(-c6ccccc6)cc5c34)cc2)cc1]

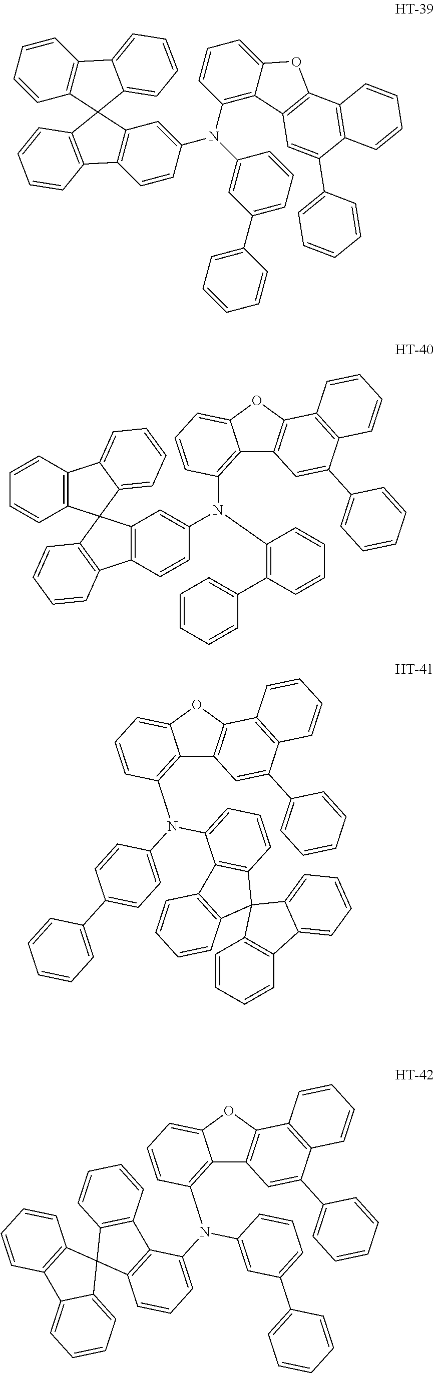 [SMILES: c1ccc(-c2cccc(N(c3ccc4c(c3)C3(c5ccccc5-c5ccccc53)c3ccccc3-4)c3cccc4oc5c6ccccc6c(-c6ccccc6)cc5c34)c2)cc1 ; c1ccc(-c2ccccc2N(c2ccc3c(c2)C2(c4ccccc4-c4ccccc42)c2ccccc2-3)c2cccc3oc4c5ccccc5c(-c5ccccc5)cc4c23)cc1 ; c1ccc(-c2ccc(N(c3cccc4c3-c3ccccc3C43c4ccccc4-c4ccccc43)c3cccc4oc5c6ccccc6c(-c6ccccc6)cc5c34)cc2)cc1 ; c1ccc(-c2cccc(N(c3cccc4c3-c3ccccc3C43c4ccccc4-c4ccccc43)c3cccc4oc5c6ccccc6c(-c6ccccc6)cc5c34)c2)cc1]

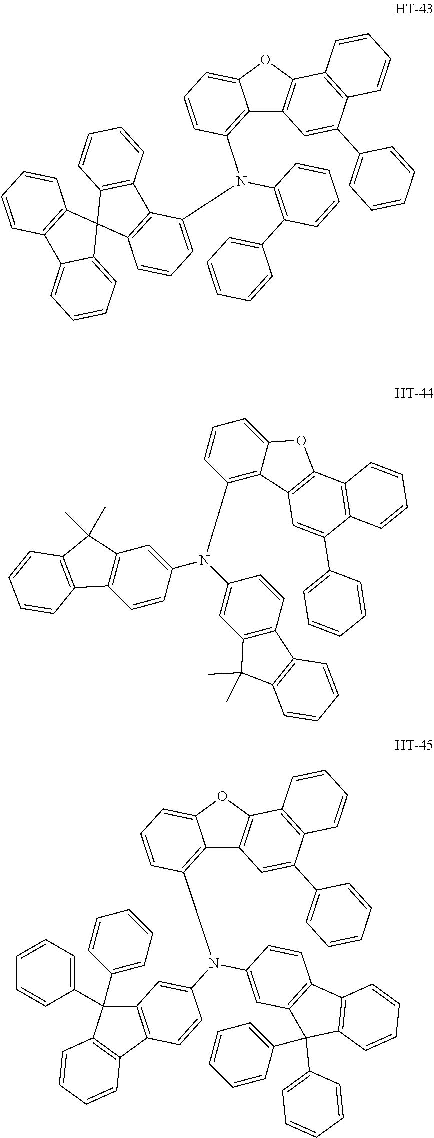 [SMILES: c1ccc(-c2ccccc2N(c2cccc3c2-c2ccccc2C32c3ccccc3-c3ccccc32)c2cccc3oc4c5ccccc5c(-c5ccccc5)cc4c23)cc1 ; CC1(C)c2ccccc2-c2ccc(N(c3ccc4c(c3)C(C)(C)c3ccccc3-4)c3cccc4oc5c6ccccc6c(-c6ccccc6)cc5c34)cc21 ; c1ccc(-c2cc3c(oc4cccc(N(c5ccc6c(c5)C(c5ccccc5)(c5ccccc5)c5ccccc5-6)c5ccc6c(c5)C(c5ccccc5)(c5ccccc5)c5ccccc5-6)c43)c3ccccc23)cc1]

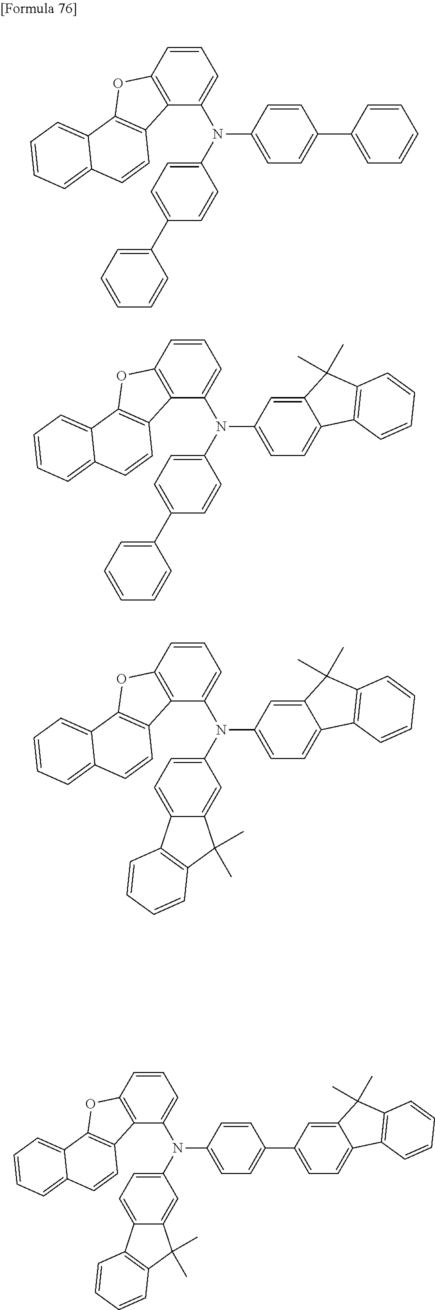

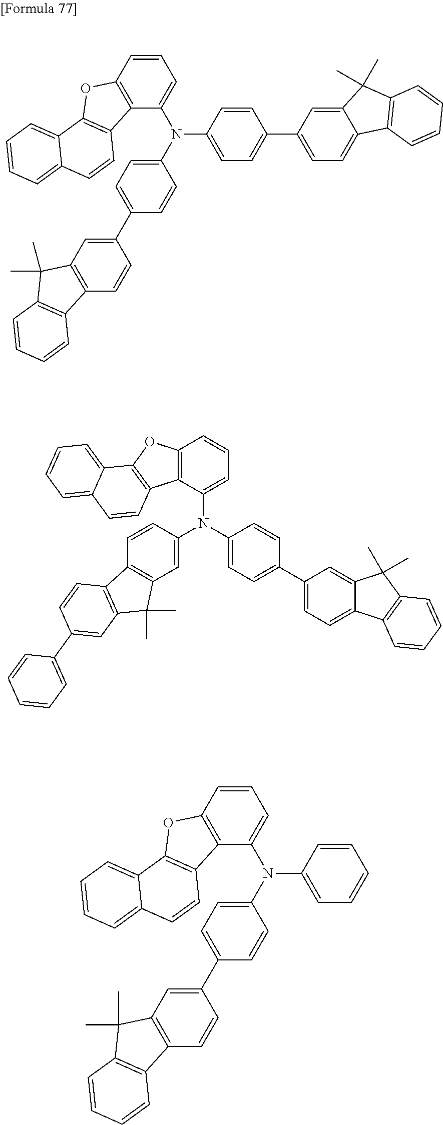 [SMILES: CC1(C)c2ccccc2-c2ccc(-c3ccc(N(c4ccc(-c5ccc6c(c5)C(C)(C)c5ccccc5-6)cc4)c4cccc5oc6c7ccccc7ccc6c45)cc3)cc21 ; CC1(C)c2ccccc2-c2ccc(-c3ccc(N(c4ccc5c(c4)C(C)(C)c4cc(-c6ccccc6)ccc4-5)c4cccc5oc6c7ccccc7ccc6c45)cc3)cc21 ; CC1(C)c2ccccc2-c2ccc(-c3ccc(N(c4ccccc4)c4cccc5oc6c7ccccc7ccc6c45)cc3)cc21]

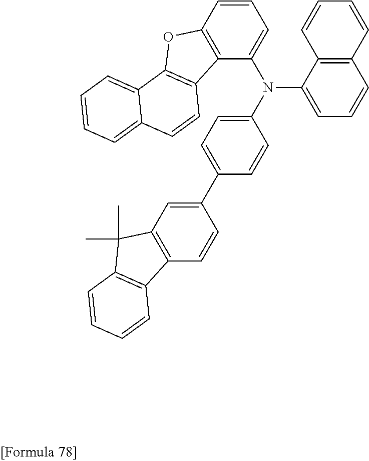 [SMILES: CC1(C)c2ccccc2-c2ccc(-c3ccc(N(c4cccc5ccccc45)c4cccc5oc6c7ccccc7ccc6c45)cc3)cc21]

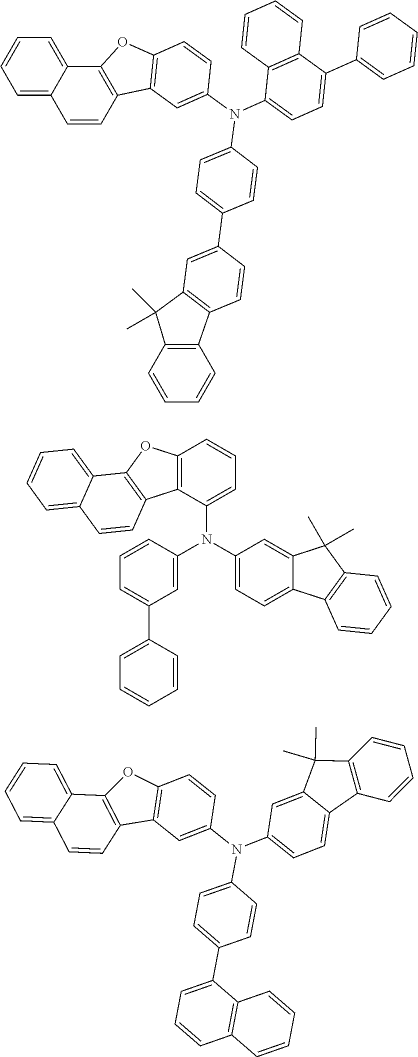

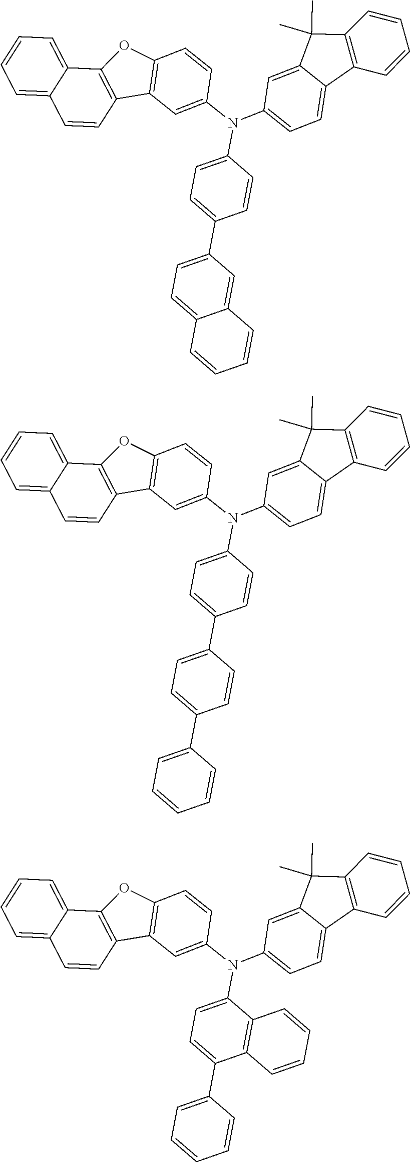 [SMILES: CC1(C)c2ccccc2-c2ccc(N(c3ccc(-c4ccc5ccccc5c4)cc3)c3ccc4oc5c6ccccc6ccc5c4c3)cc21 ; CC1(C)c2ccccc2-c2ccc(N(c3ccc(-c4ccc(-c5ccccc5)cc4)cc3)c3ccc4oc5c6ccccc6ccc5c4c3)cc21 ; CC1(C)c2ccccc2-c2ccc(N(c3ccc4oc5c6ccccc6ccc5c4c3)c3ccc(-c4ccccc4)c4ccccc34)cc21]

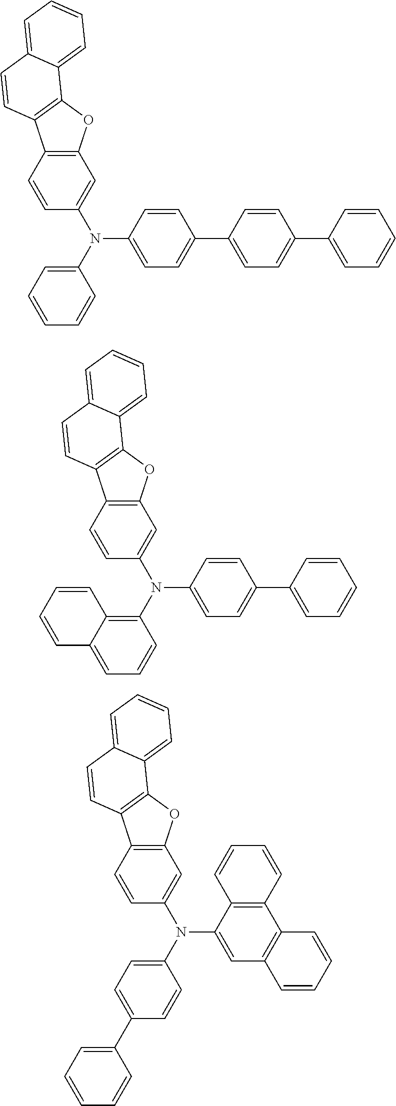

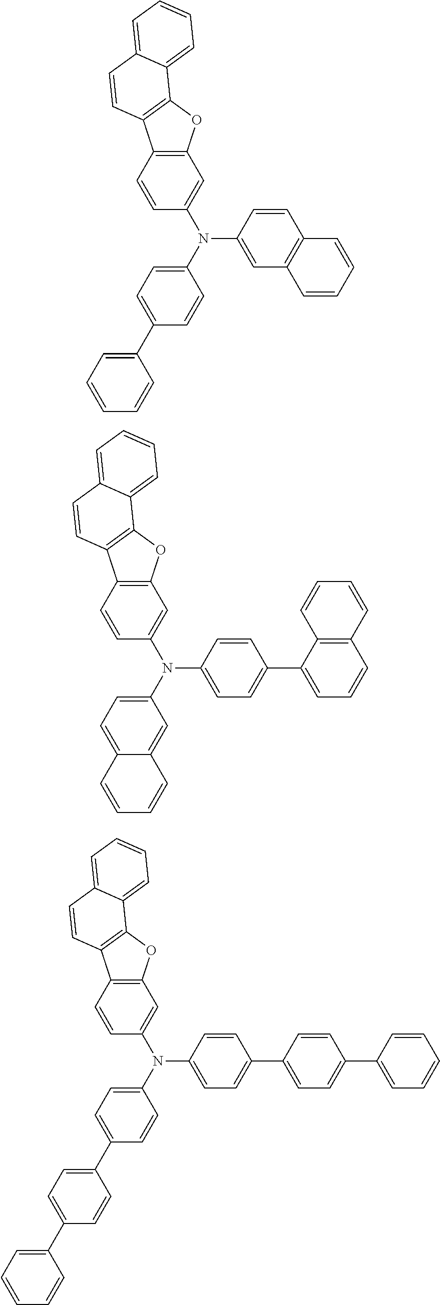

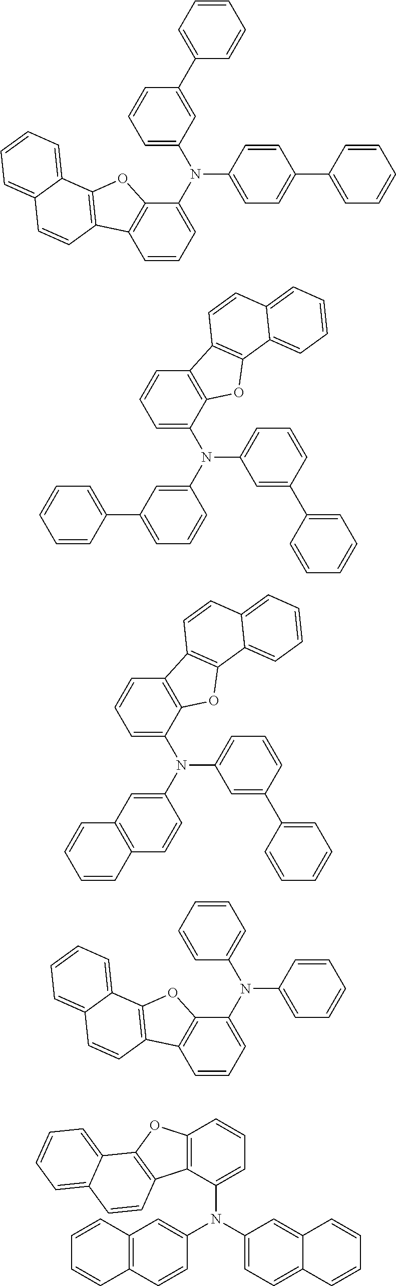

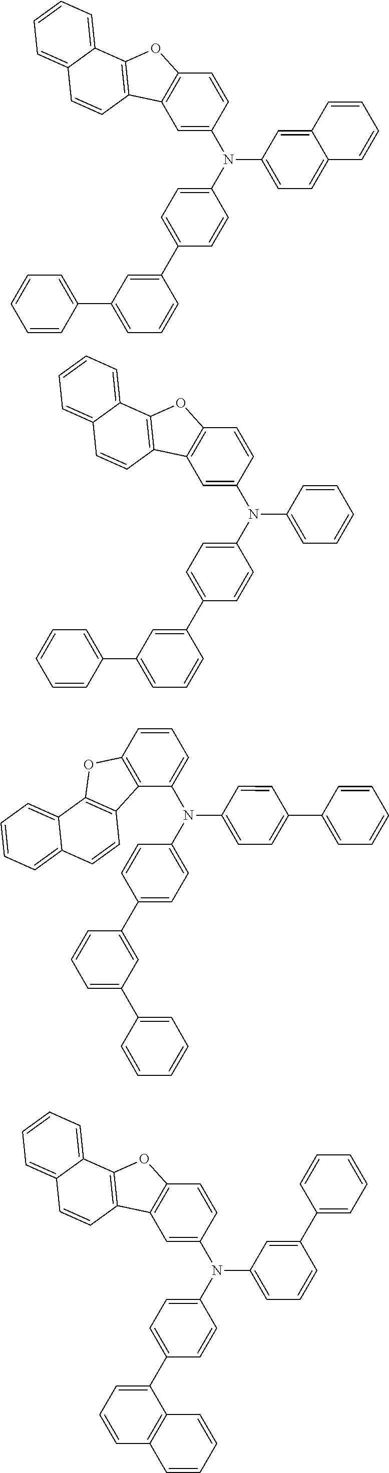

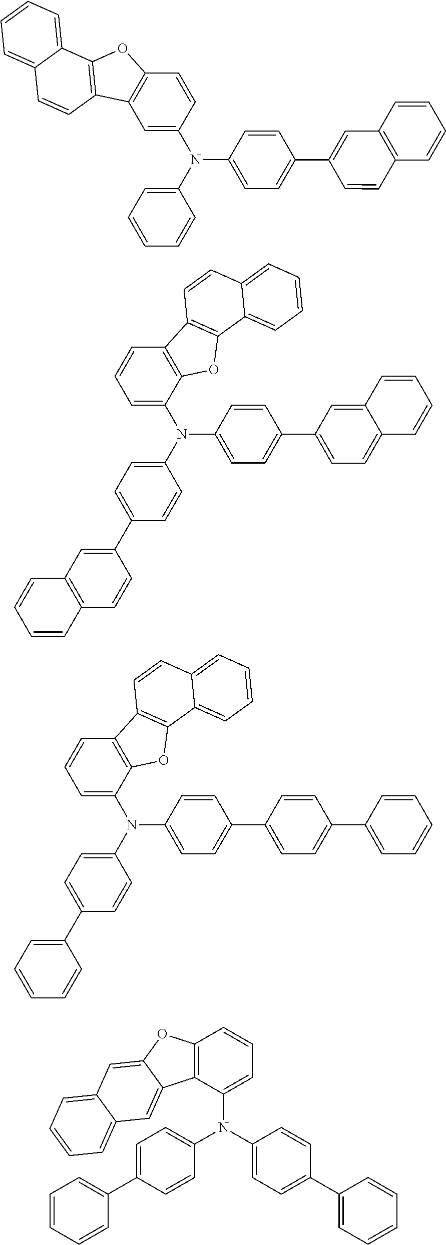

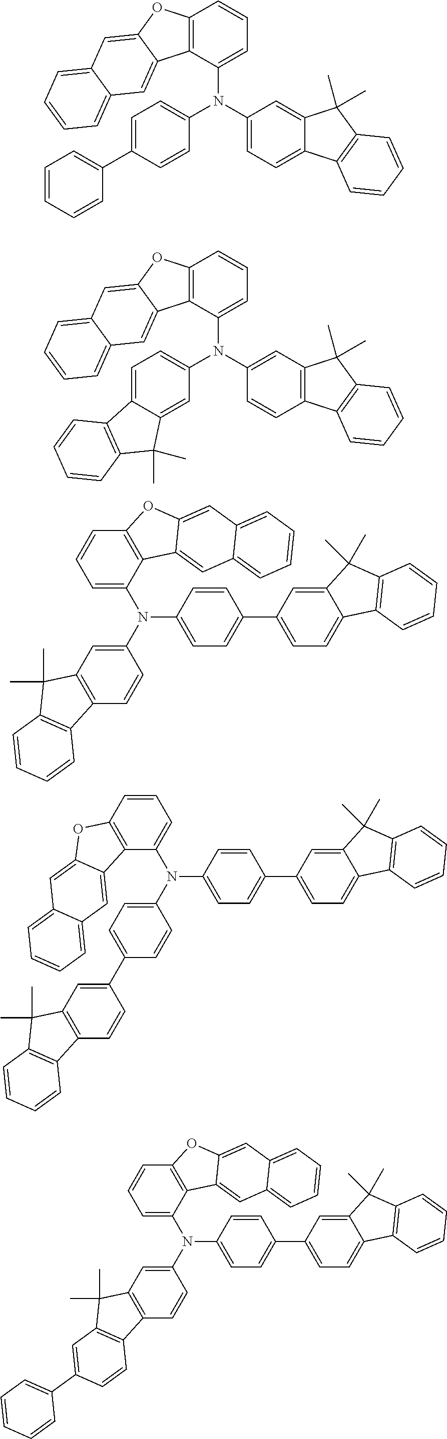

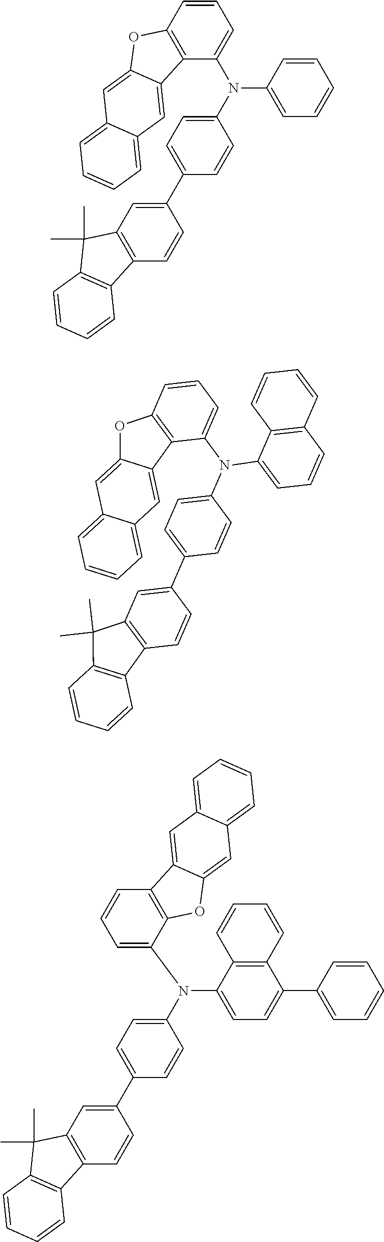 [SMILES: CC1(C)c2ccccc2-c2ccc(-c3ccc(N(c4ccccc4)c4cccc5oc6cc7ccccc7cc6c45)cc3)cc21 ; CC1(C)c2ccccc2-c2ccc(-c3ccc(N(c4cccc5ccccc45)c4cccc5oc6cc7ccccc7cc6c45)cc3)cc21 ; CC1(C)c2ccccc2-c2ccc(-c3ccc(N(c4ccc(-c5ccccc5)c5ccccc45)c4cccc5c4oc4cc6ccccc6cc45)cc3)cc21]

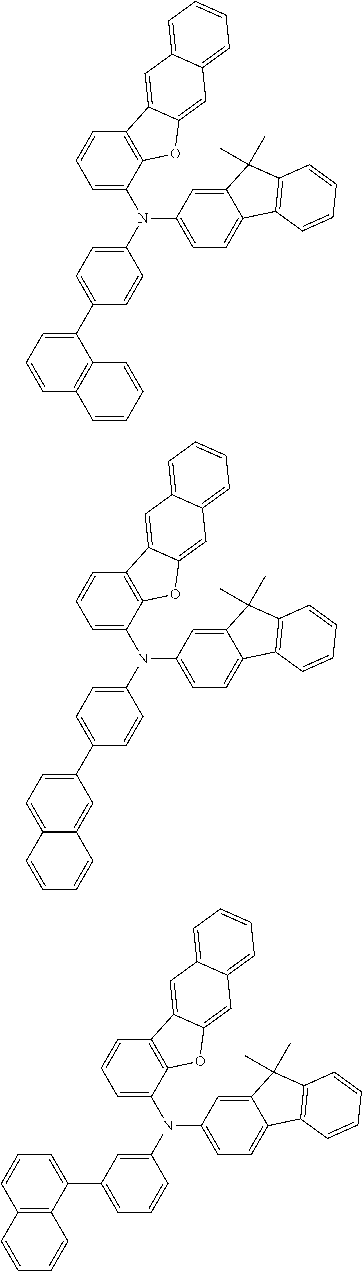 [SMILES: CC1(C)c2ccccc2-c2ccc(N(c3ccc(-c4cccc5ccccc45)cc3)c3cccc4c3oc3cc5ccccc5cc34)cc21 ; CC1(C)c2ccccc2-c2ccc(N(c3ccc(-c4ccc5ccccc5c4)cc3)c3cccc4c3oc3cc5ccccc5cc34)cc21 ; CC1(C)c2ccccc2-c2ccc(N(c3cccc(-c4cccc5ccccc45)c3)c3cccc4c3oc3cc5ccccc5cc34)cc21]

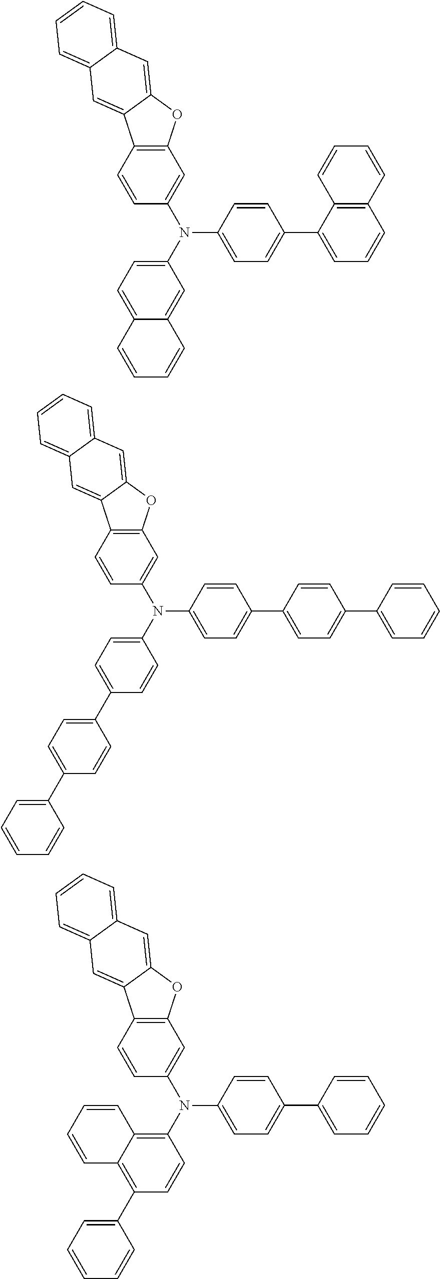

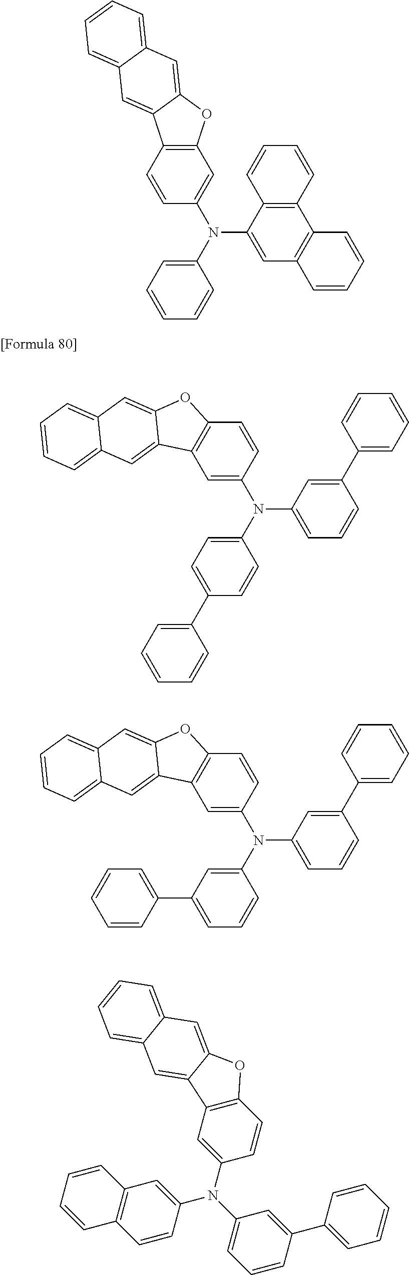 [SMILES: c1ccc(N(c2ccc3c(c2)oc2cc4ccccc4cc23)c2cc3ccccc3c3ccccc23)cc1 ; c1ccc(-c2ccc(N(c3cccc(-c4ccccc4)c3)c3ccc4oc5cc6ccccc6cc5c4c3)cc2)cc1 ; c1ccc(-c2cccc(N(c3cccc(-c4ccccc4)c3)c3ccc4oc5cc6ccccc6cc5c4c3)c2)cc1 ; c1ccc(-c2cccc(N(c3ccc4ccccc4c3)c3ccc4oc5cc6ccccc6cc5c4c3)c2)cc1]

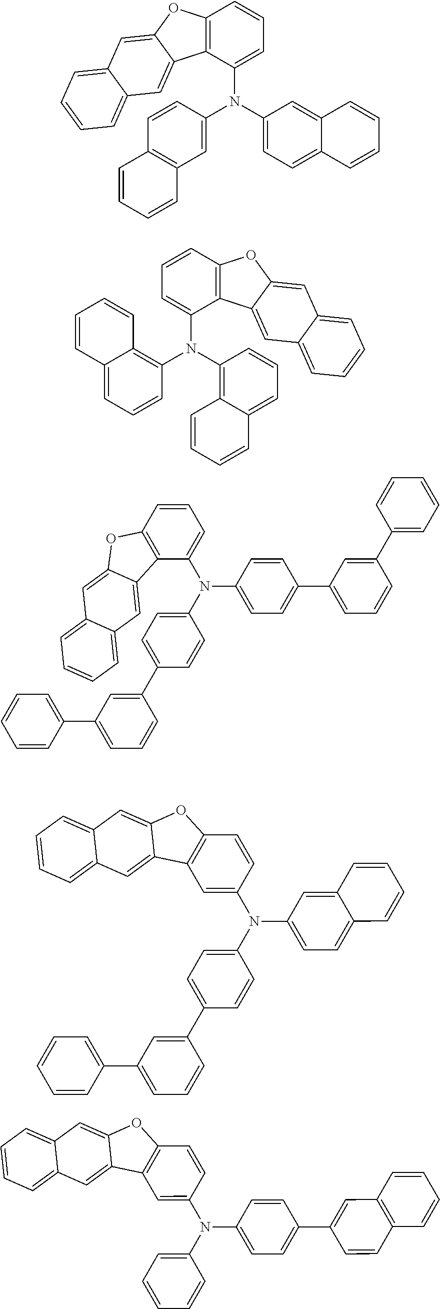

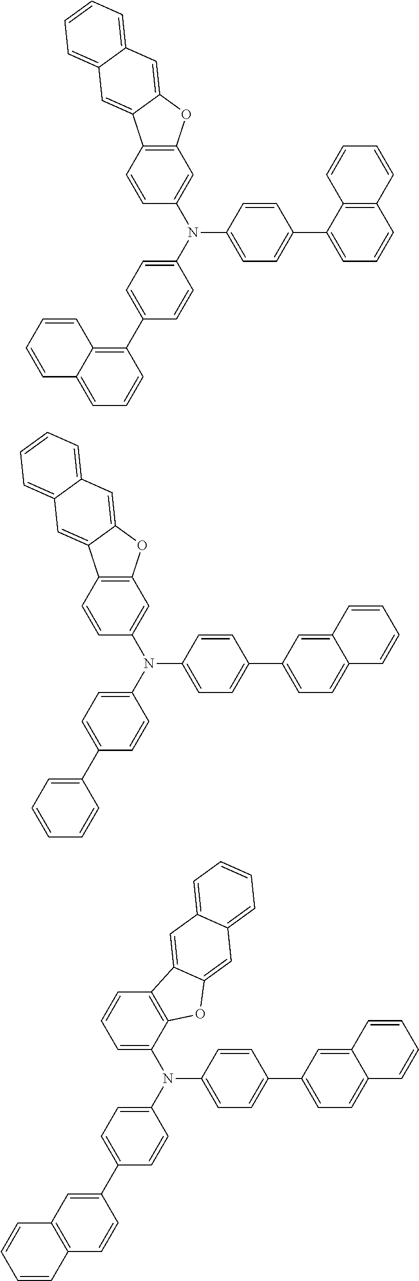

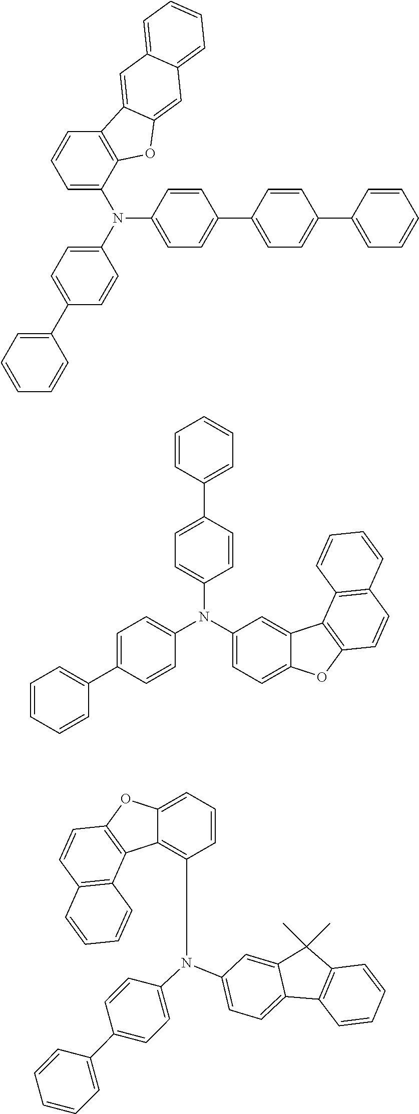 [SMILES: c1ccc(-c2ccc(-c3ccc(N(c4ccc(-c5ccccc5)cc4)c4cccc5c4oc4cc6ccccc6cc45)cc3)cc2)cc1 ; c1ccc(-c2ccc(N(c3ccc(-c4ccccc4)cc3)c3ccc4oc5ccc6ccccc6c5c4c3)cc2)cc1 ; CC1(C)c2ccccc2-c2ccc(N(c3ccc(-c4ccccc4)cc3)c3cccc4oc5ccc6ccccc6c5c34)cc21]

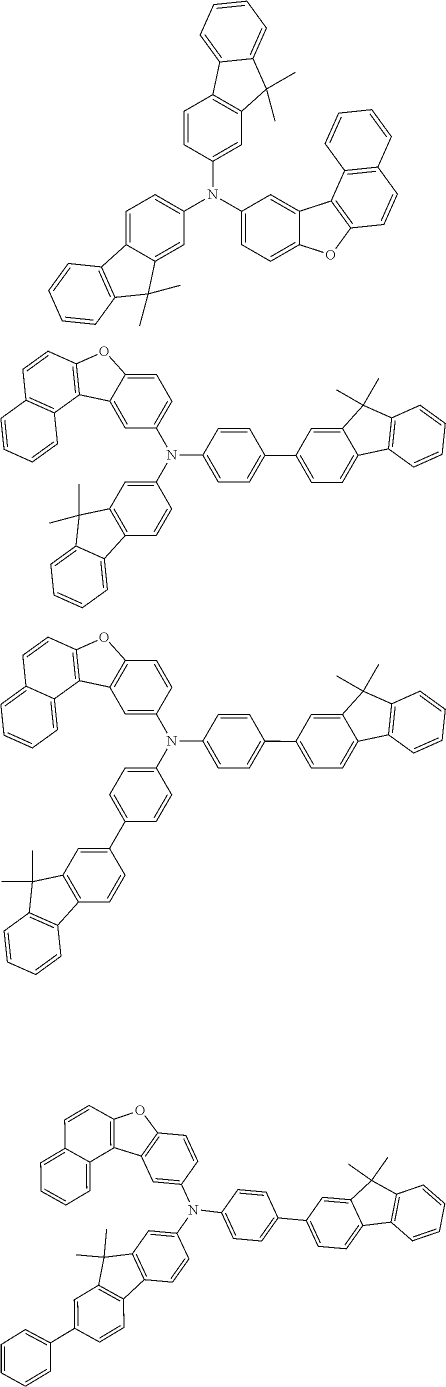

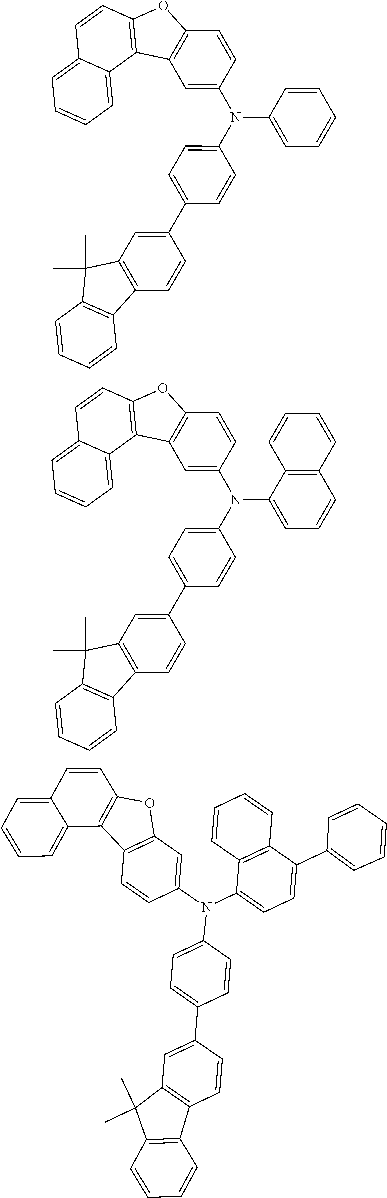

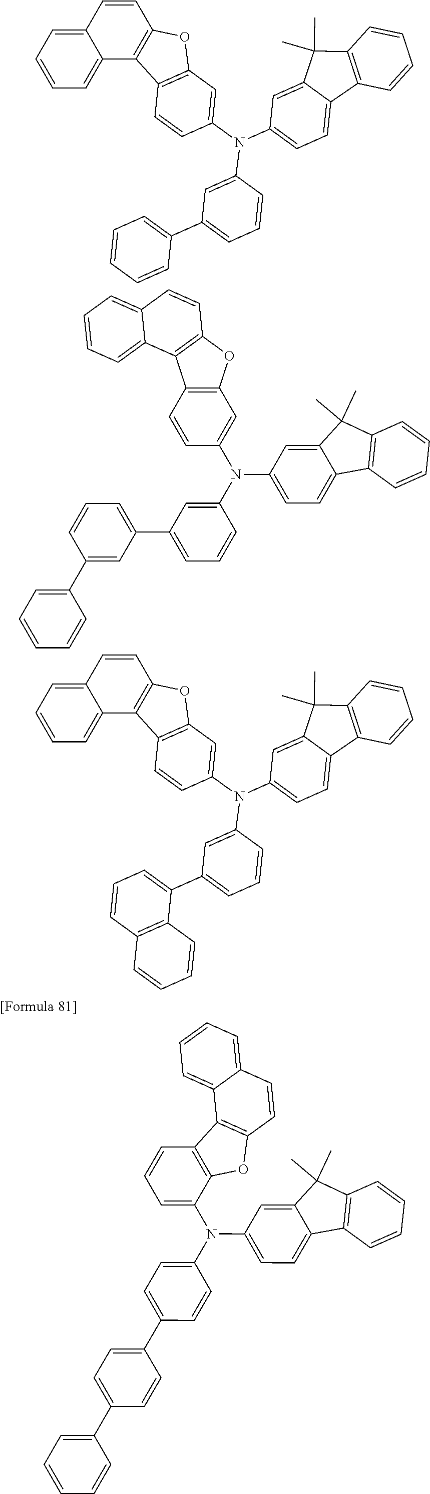

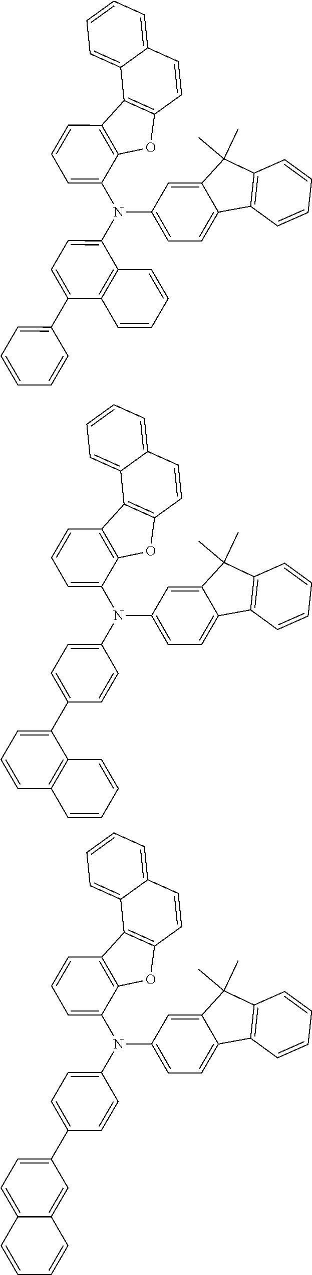

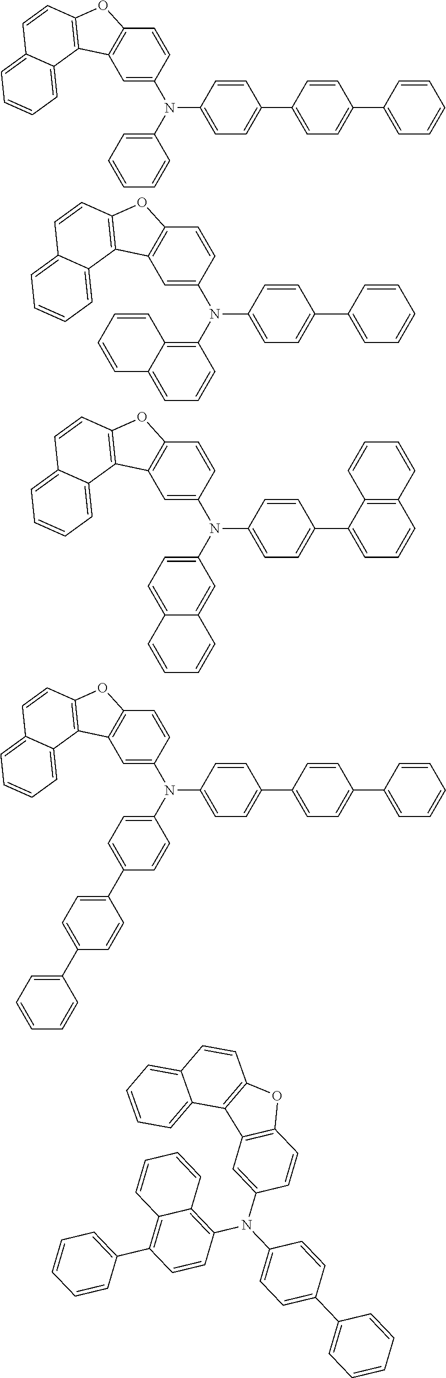

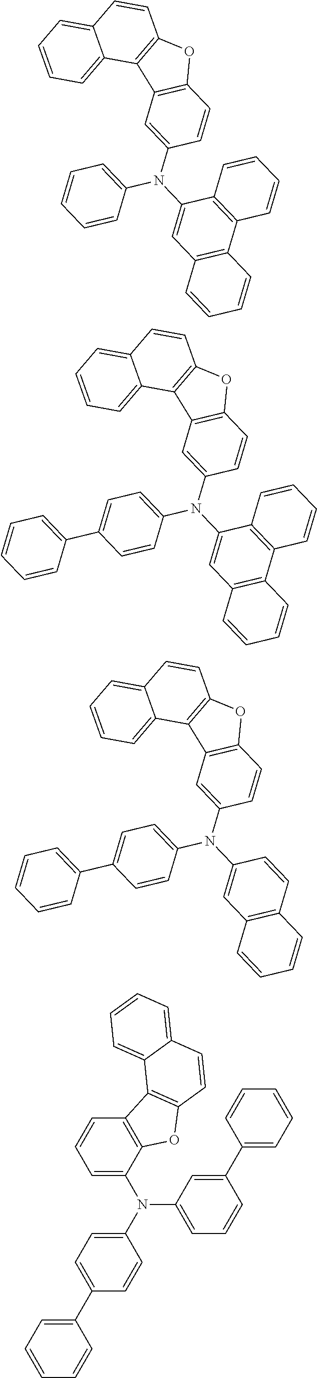

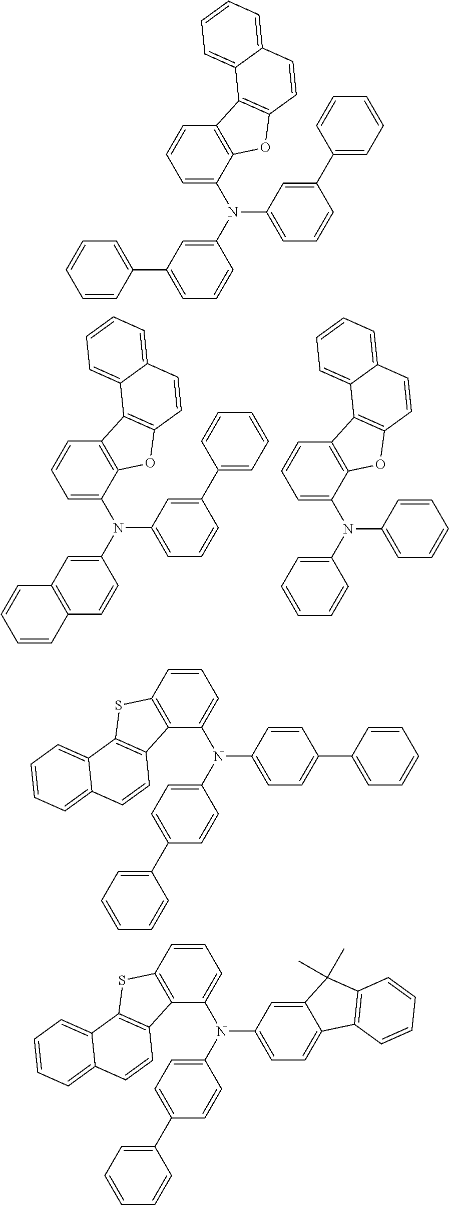

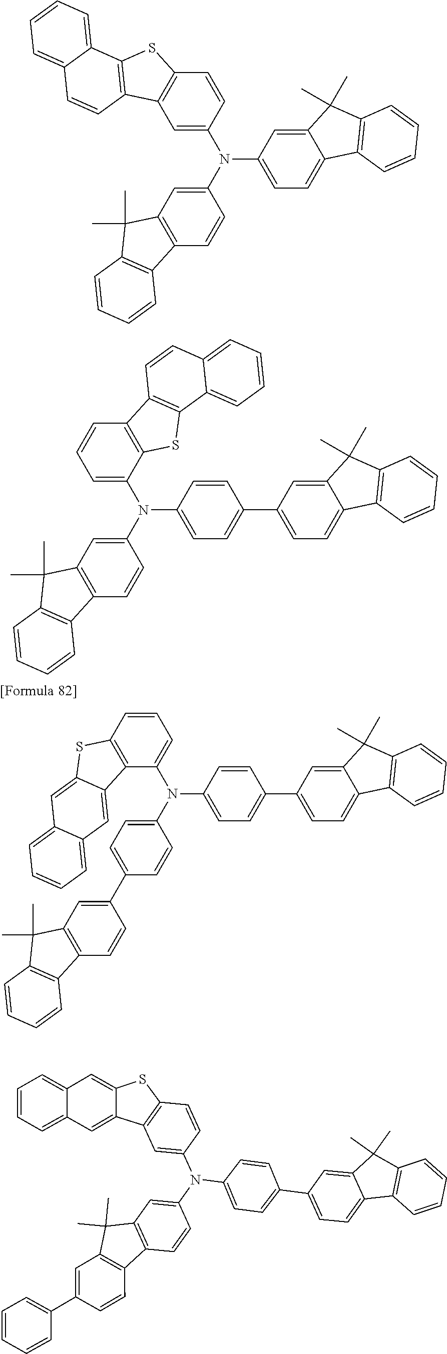 [SMILES: CC1(C)c2ccccc2-c2ccc(N(c3ccc4c(c3)C(C)(C)c3ccccc3-4)c3ccc4sc5c6ccccc6ccc5c4c3)cc21 ; CC1(C)c2ccccc2-c2ccc(-c3ccc(N(c4ccc5c(c4)C(C)(C)c4ccccc4-5)c4cccc5c4sc4c6ccccc6ccc54)cc3)cc21 ; CC1(C)c2ccccc2-c2ccc(-c3ccc(N(c4ccc(-c5ccc6c(c5)C(C)(C)c5ccccc5-6)cc4)c4cccc5sc6cc7ccccc7cc6c45)cc3)cc21 ; CC1(C)c2ccccc2-c2ccc(-c3ccc(N(c4ccc5c(c4)C(C)(C)c4cc(-c6ccccc6)ccc4-5)c4ccc5sc6cc7ccccc7cc6c5c4)cc3)cc21]

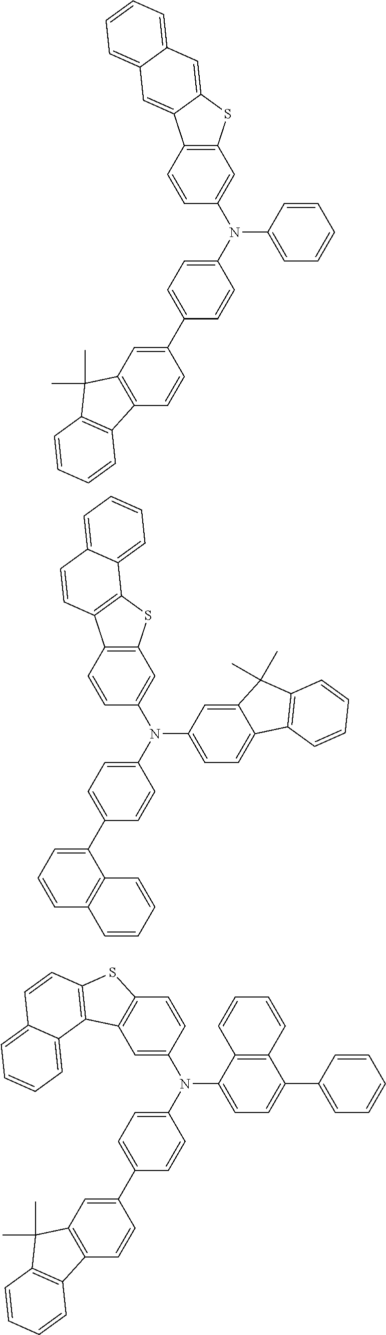

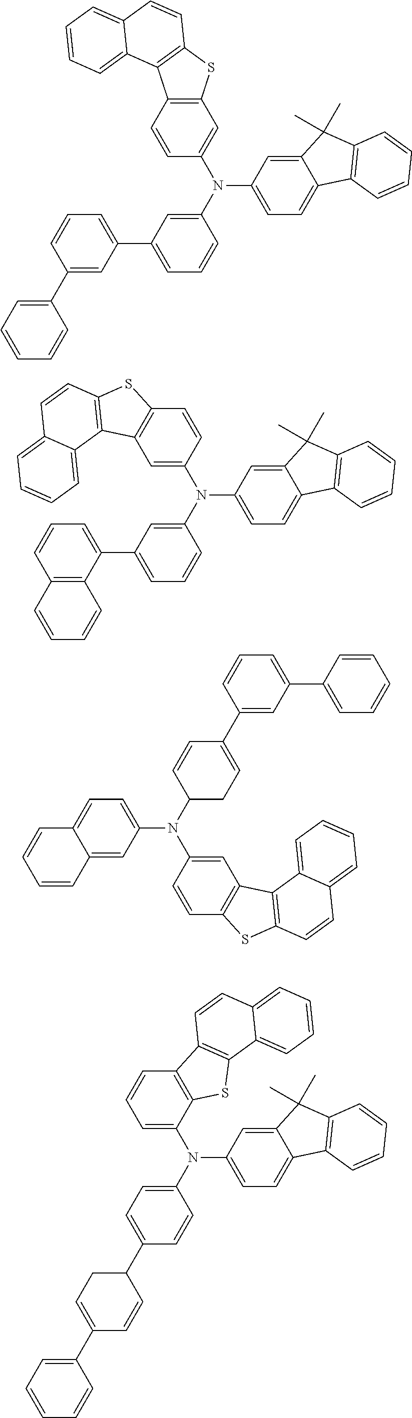

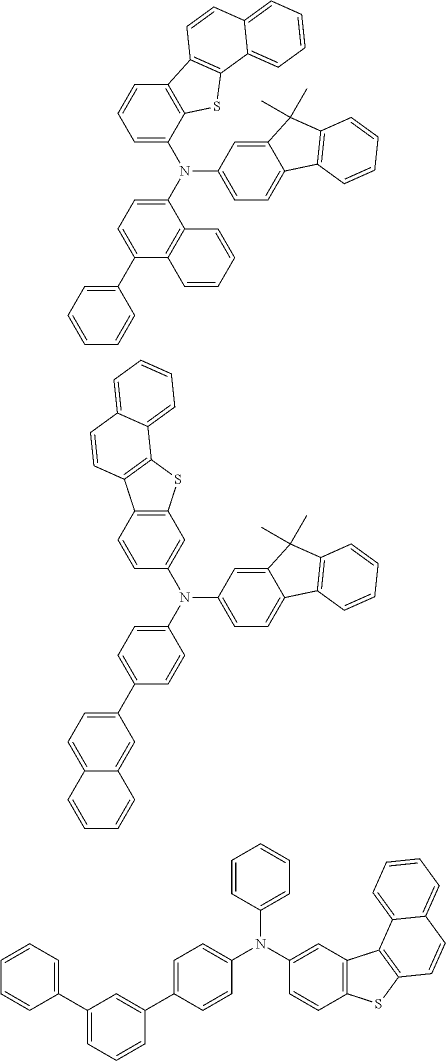

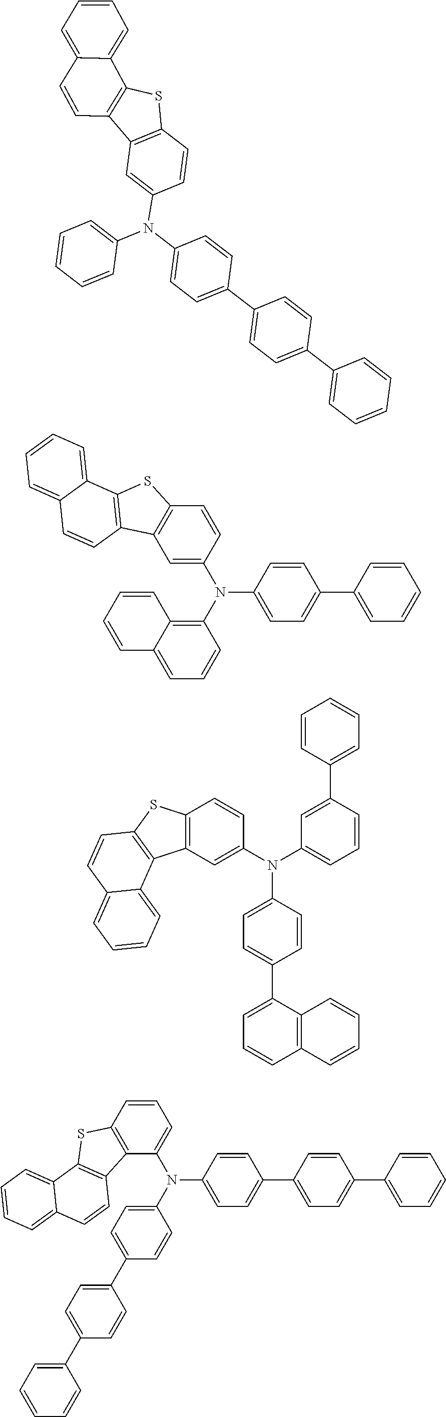 [SMILES: c1ccc(-c2ccc(-c3ccc(N(c4ccccc4)c4ccc5sc6c7ccccc7ccc6c5c4)cc3)cc2)cc1 ; c1ccc(-c2ccc(N(c3ccc4sc5c6ccccc6ccc5c4c3)c3cccc4ccccc34)cc2)cc1 ; c1ccc(-c2cccc(N(c3ccc(-c4cccc5ccccc45)cc3)c3ccc4sc5ccc6ccccc6c5c4c3)c2)cc1 ; c1ccc(-c2ccc(-c3ccc(N(c4ccc(-c5ccc(-c6ccccc6)cc5)cc4)c4cccc5sc6c7ccccc7ccc6c45)cc3)cc2)cc1]

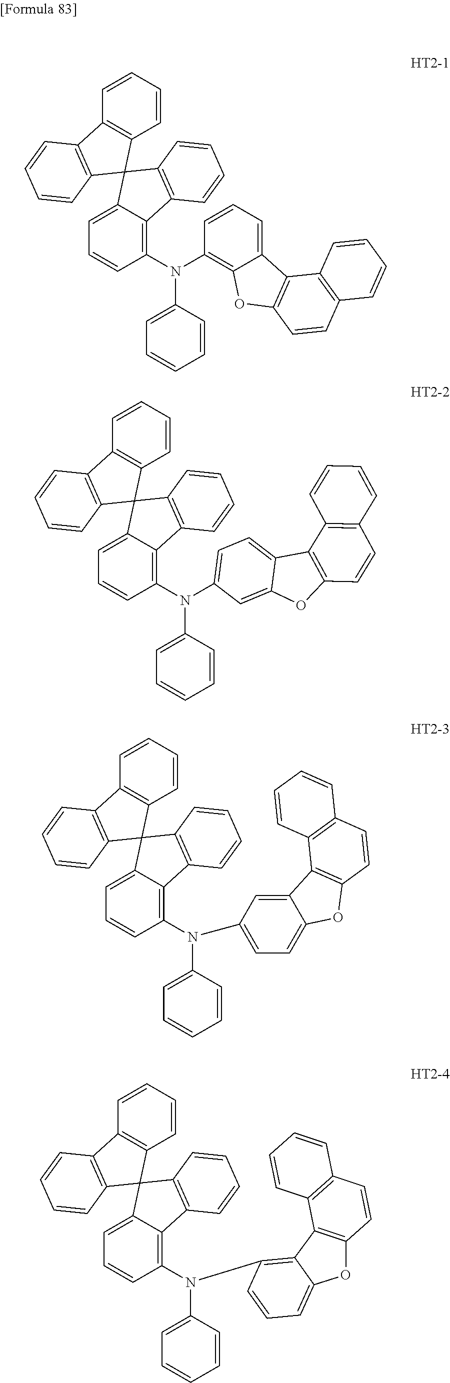

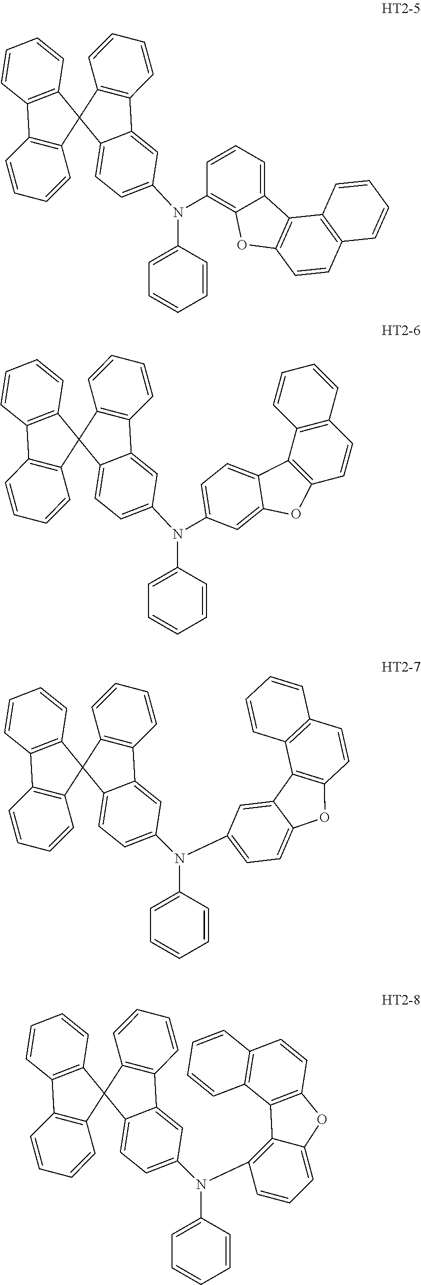 [SMILES: c1ccc(N(c2ccc3c(c2)-c2ccccc2C32c3ccccc3-c3ccccc32)c2cccc3c2oc2ccc4ccccc4c23)cc1 ; c1ccc(N(c2ccc3c(c2)-c2ccccc2C32c3ccccc3-c3ccccc32)c2ccc3c(c2)oc2ccc4ccccc4c23)cc1 ; c1ccc(N(c2ccc3c(c2)-c2ccccc2C32c3ccccc3-c3ccccc32)c2ccc3oc4ccc5ccccc5c4c3c2)cc1 ; c1ccc(N(c2ccc3c(c2)-c2ccccc2C32c3ccccc3-c3ccccc32)c2cccc3oc4ccc5ccccc5c4c23)cc1]

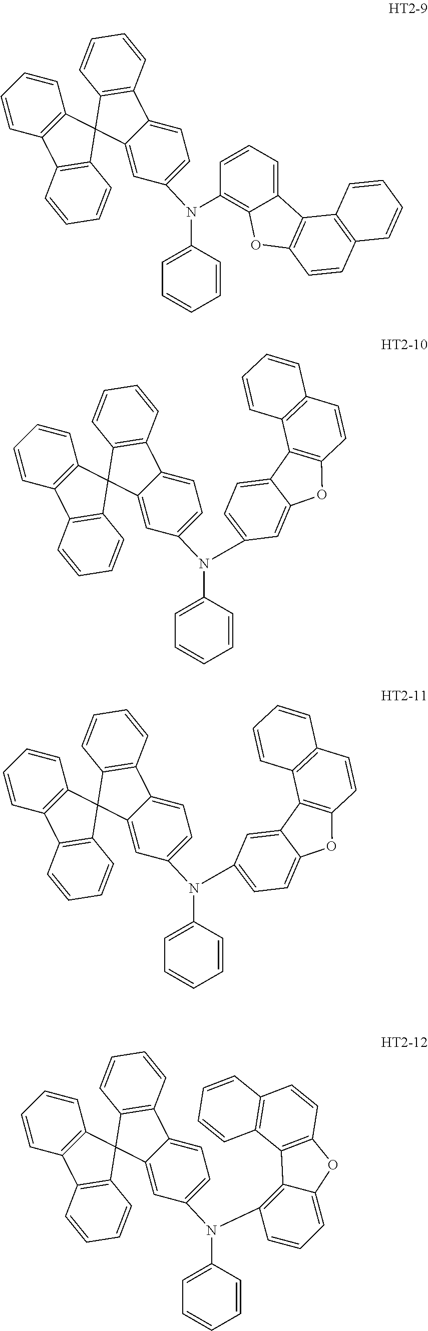 [SMILES: c1ccc(N(c2ccc3c(c2)C2(c4ccccc4-c4ccccc42)c2ccccc2-3)c2cccc3c2oc2ccc4ccccc4c23)cc1 ; c1ccc(N(c2ccc3c(c2)C2(c4ccccc4-c4ccccc42)c2ccccc2-3)c2ccc3c(c2)oc2ccc4ccccc4c23)cc1 ; c1ccc(N(c2ccc3c(c2)C2(c4ccccc4-c4ccccc42)c2ccccc2-3)c2ccc3oc4ccc5ccccc5c4c3c2)cc1 ; c1ccc(N(c2ccc3c(c2)C2(c4ccccc4-c4ccccc42)c2ccccc2-3)c2cccc3oc4ccc5ccccc5c4c23)cc1]

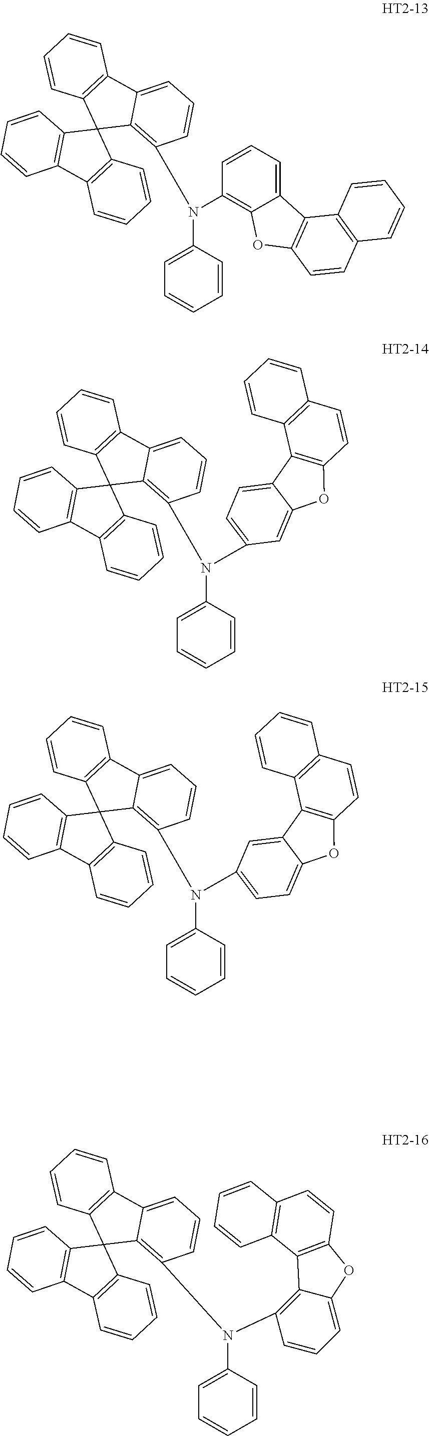 [SMILES: c1ccc(N(c2cccc3c2C2(c4ccccc4-c4ccccc42)c2ccccc2-3)c2cccc3c2oc2ccc4ccccc4c23)cc1 ; c1ccc(N(c2ccc3c(c2)oc2ccc4ccccc4c23)c2cccc3c2C2(c4ccccc4-c4ccccc42)c2ccccc2-3)cc1 ; c1ccc(N(c2ccc3oc4ccc5ccccc5c4c3c2)c2cccc3c2C2(c4ccccc4-c4ccccc42)c2ccccc2-3)cc1 ; c1ccc(N(c2cccc3c2C2(c4ccccc4-c4ccccc42)c2ccccc2-3)c2cccc3oc4ccc5ccccc5c4c23)cc1]

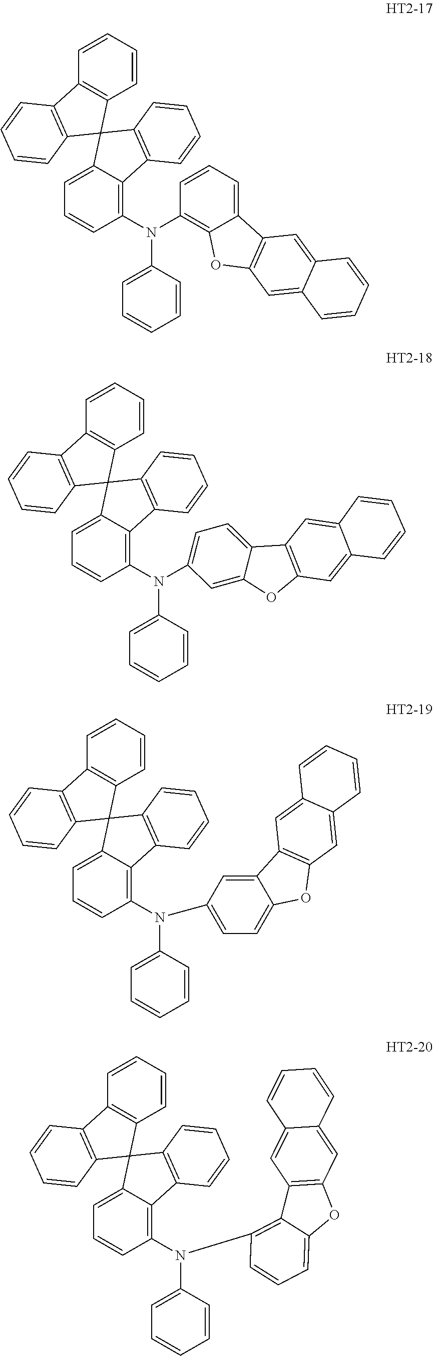

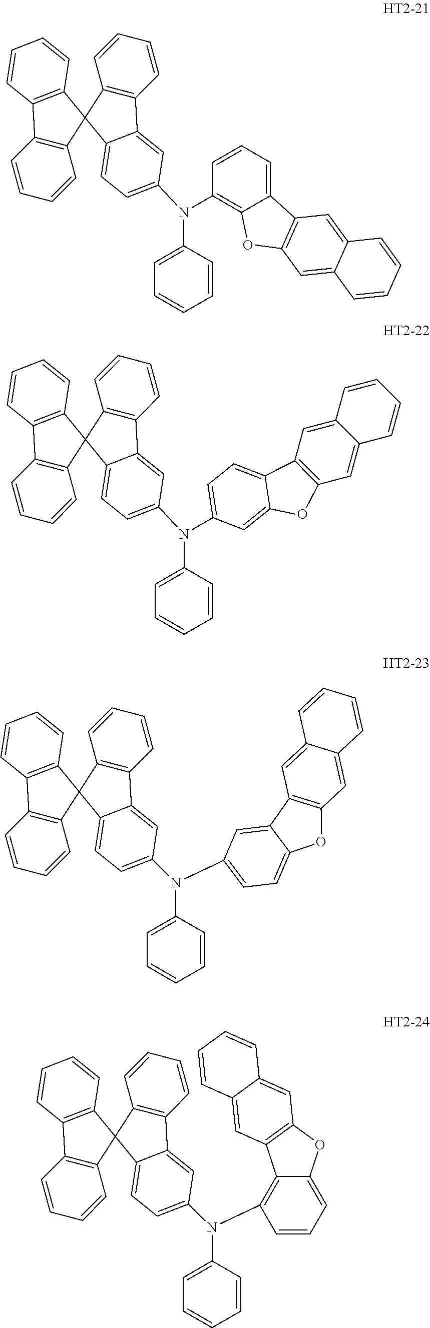 [SMILES: c1ccc(N(c2ccc3c(c2)-c2ccccc2C32c3ccccc3-c3ccccc32)c2cccc3c2oc2cc4ccccc4cc23)cc1 ; c1ccc(N(c2ccc3c(c2)-c2ccccc2C32c3ccccc3-c3ccccc32)c2ccc3c(c2)oc2cc4ccccc4cc23)cc1 ; c1ccc(N(c2ccc3c(c2)-c2ccccc2C32c3ccccc3-c3ccccc32)c2ccc3oc4cc5ccccc5cc4c3c2)cc1 ; c1ccc(N(c2ccc3c(c2)-c2ccccc2C32c3ccccc3-c3ccccc32)c2cccc3oc4cc5ccccc5cc4c23)cc1]

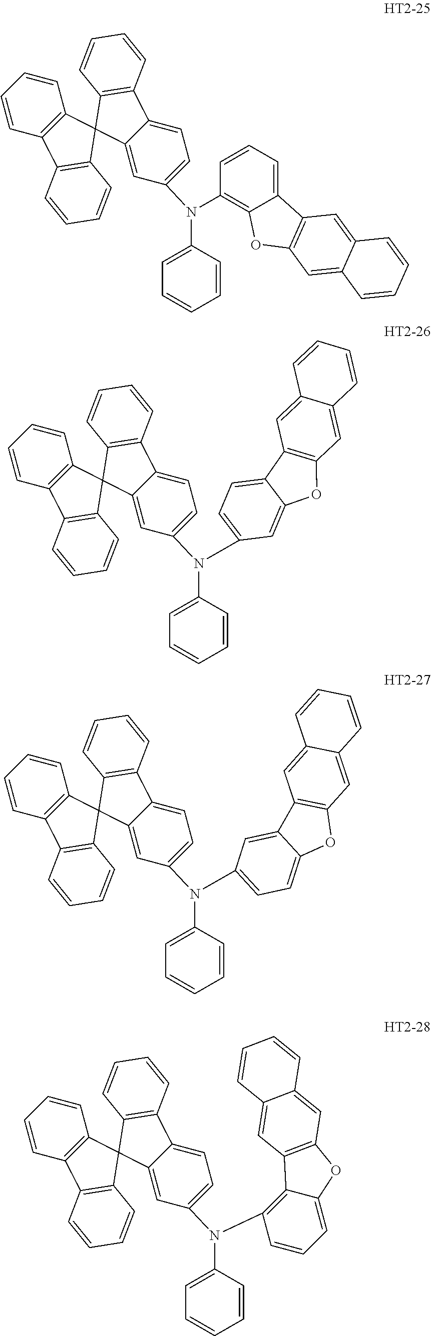 [SMILES: c1ccc(N(c2ccc3c(c2)C2(c4ccccc4-c4ccccc42)c2ccccc2-3)c2cccc3c2oc2cc4ccccc4cc23)cc1 ; c1ccc(N(c2ccc3c(c2)C2(c4ccccc4-c4ccccc42)c2ccccc2-3)c2ccc3c(c2)oc2cc4ccccc4cc23)cc1 ; c1ccc(N(c2ccc3c(c2)C2(c4ccccc4-c4ccccc42)c2ccccc2-3)c2ccc3oc4cc5ccccc5cc4c3c2)cc1 ; c1ccc(N(c2ccc3c(c2)C2(c4ccccc4-c4ccccc42)c2ccccc2-3)c2cccc3oc4cc5ccccc5cc4c23)cc1]

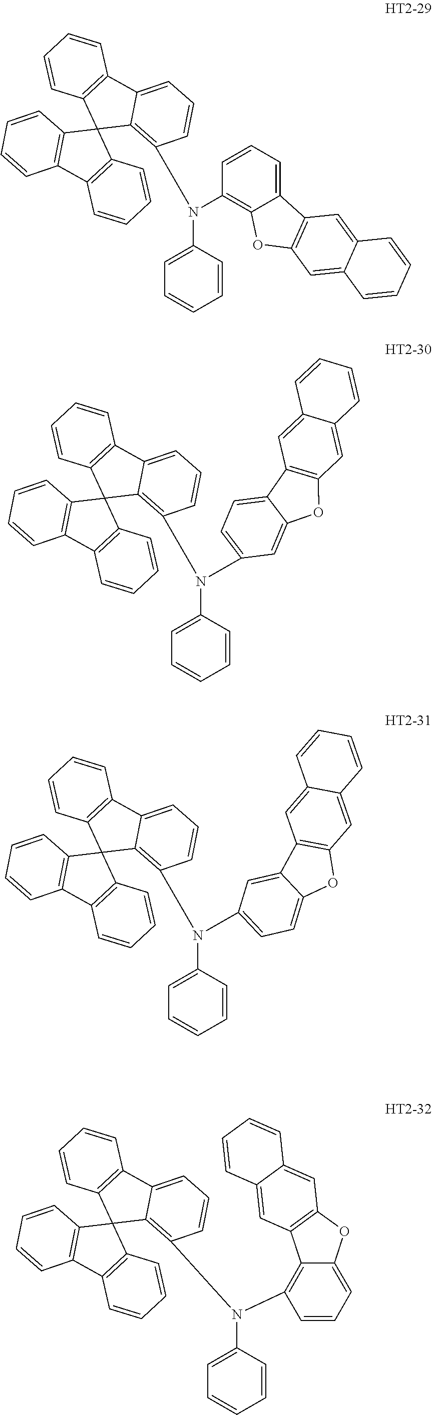 [SMILES: c1ccc(N(c2cccc3c2C2(c4ccccc4-c4ccccc42)c2ccccc2-3)c2cccc3c2oc2cc4ccccc4cc23)cc1 ; c1ccc(N(c2ccc3c(c2)oc2cc4ccccc4cc23)c2cccc3c2C2(c4ccccc4-c4ccccc42)c2ccccc2-3)cc1 ; c1ccc(N(c2ccc3oc4cc5ccccc5cc4c3c2)c2cccc3c2C2(c4ccccc4-c4ccccc42)c2ccccc2-3)cc1 ; c1ccc(N(c2cccc3c2C2(c4ccccc4-c4ccccc42)c2ccccc2-3)c2cccc3oc4cc5ccccc5cc4c23)cc1]

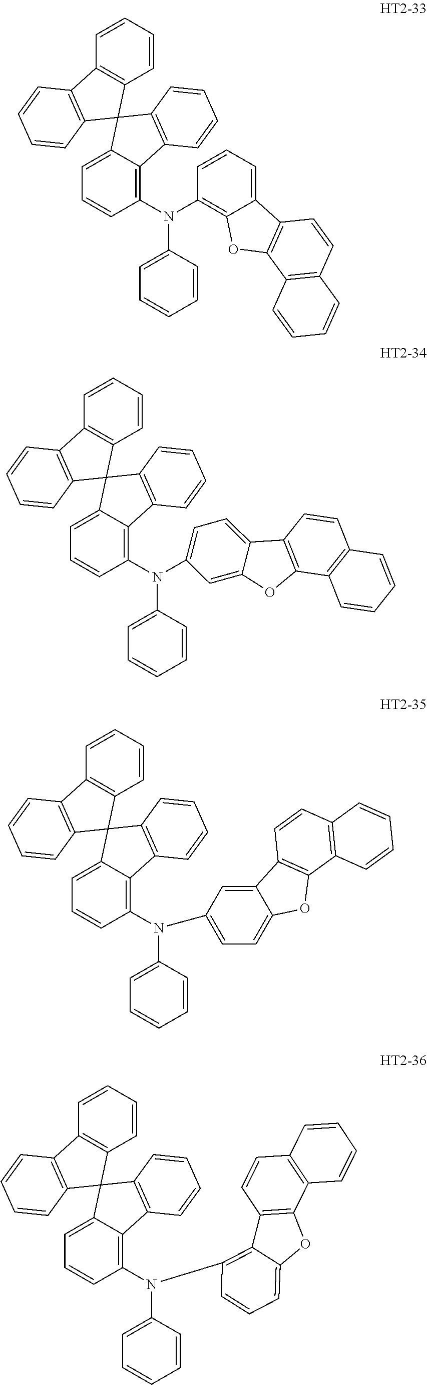 [SMILES: c1ccc(N(c2cccc3c2-c2ccccc2C32c3ccccc3-c3ccccc32)c2cccc3c2oc2c4ccccc4ccc32)cc1 ; c1ccc(N(c2ccc3c(c2)oc2c4ccccc4ccc32)c2cccc3c2-c2ccccc2C32c3ccccc3-c3ccccc32)cc1 ; c1ccc(N(c2ccc3oc4c5ccccc5ccc4c3c2)c2cccc3c2-c2ccccc2C32c3ccccc3-c3ccccc32)cc1 ; c1ccc(N(c2cccc3c2-c2ccccc2C32c3ccccc3-c3ccccc32)c2cccc3oc4c5ccccc5ccc4c23)cc1]

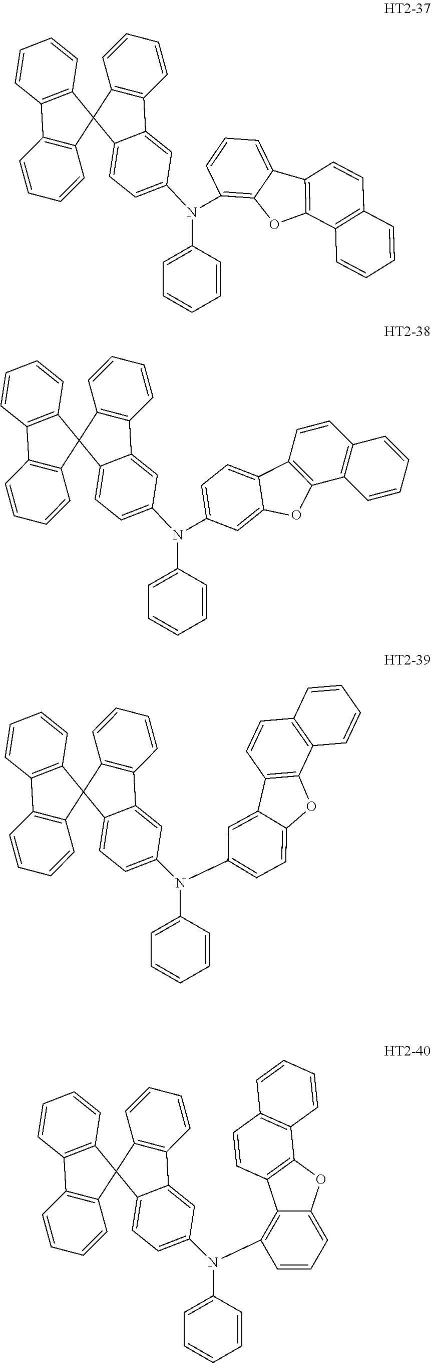 [SMILES: c1ccc(N(c2ccc3c(c2)-c2ccccc2C32c3ccccc3-c3ccccc32)c2cccc3c2oc2c4ccccc4ccc32)cc1 ; c1ccc(N(c2ccc3c(c2)-c2ccccc2C32c3ccccc3-c3ccccc32)c2ccc3c(c2)oc2c4ccccc4ccc32)cc1 ; c1ccc(N(c2ccc3c(c2)-c2ccccc2C32c3ccccc3-c3ccccc32)c2ccc3oc4c5ccccc5ccc4c3c2)cc1 ; c1ccc(N(c2ccc3c(c2)-c2ccccc2C32c3ccccc3-c3ccccc32)c2cccc3oc4c5ccccc5ccc4c23)cc1]

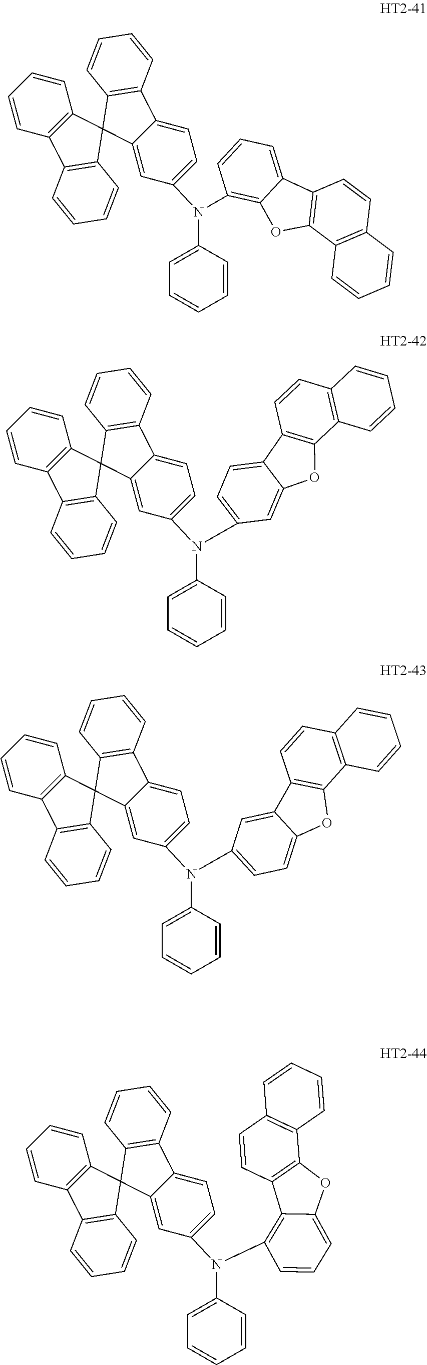 [SMILES: c1ccc(N(c2ccc3c(c2)C2(c4ccccc4-c4ccccc42)c2ccccc2-3)c2cccc3c2oc2c4ccccc4ccc32)cc1 ; c1ccc(N(c2ccc3c(c2)C2(c4ccccc4-c4ccccc42)c2ccccc2-3)c2ccc3c(c2)oc2c4ccccc4ccc32)cc1 ; c1ccc(N(c2ccc3c(c2)C2(c4ccccc4-c4ccccc42)c2ccccc2-3)c2ccc3oc4c5ccccc5ccc4c3c2)cc1 ; c1ccc(N(c2ccc3c(c2)C2(c4ccccc4-c4ccccc42)c2ccccc2-3)c2cccc3oc4c5ccccc5ccc4c23)cc1]

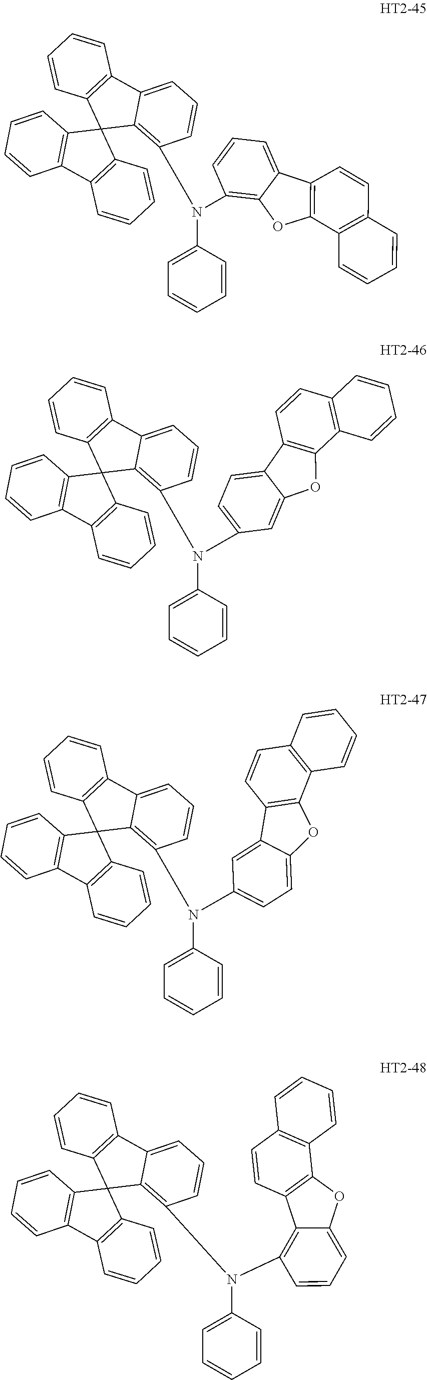 [SMILES: c1ccc(N(c2cccc3c2C2(c4ccccc4-c4ccccc42)c2ccccc2-3)c2cccc3c2oc2c4ccccc4ccc32)cc1 ; c1ccc(N(c2ccc3c(c2)oc2c4ccccc4ccc32)c2cccc3c2C2(c4ccccc4-c4ccccc42)c2ccccc2-3)cc1 ; c1ccc(N(c2ccc3oc4c5ccccc5ccc4c3c2)c2cccc3c2C2(c4ccccc4-c4ccccc42)c2ccccc2-3)cc1 ; c1ccc(N(c2cccc3c2C2(c4ccccc4-c4ccccc42)c2ccccc2-3)c2cccc3oc4c5ccccc5ccc4c23)cc1]

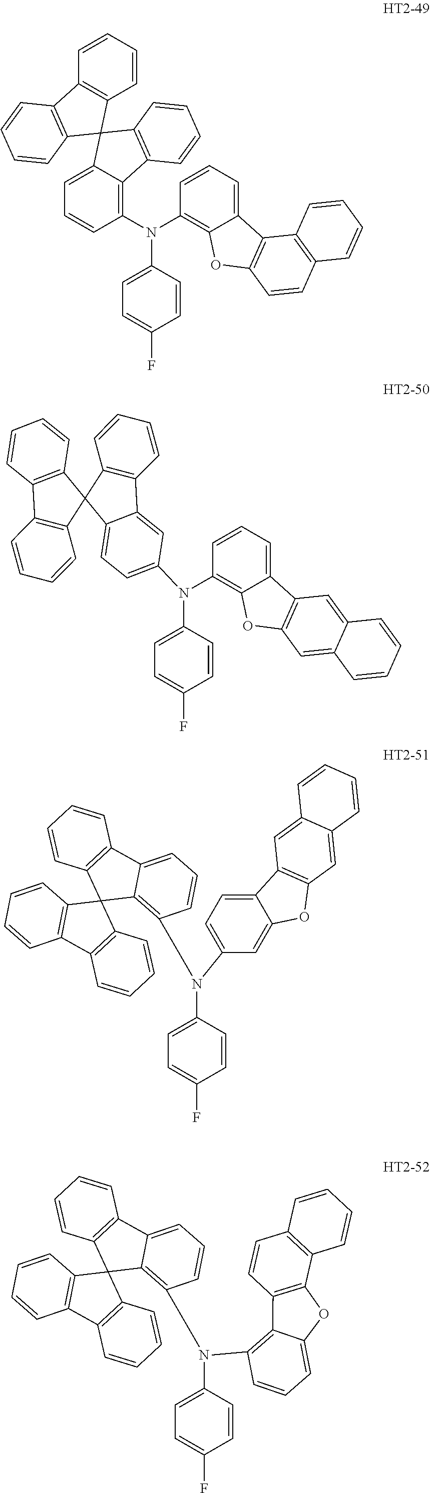 [SMILES: Fc1ccc(N(c2cccc3c2-c2ccccc2C32c3ccccc3-c3ccccc32)c2cccc3c2oc2ccc4ccccc4c23)cc1 ; Fc1ccc(N(c2ccc3c(c2)-c2ccccc2C32c3ccccc3-c3ccccc32)c2cccc3c2oc2cc4ccccc4cc23)cc1 ; Fc1ccc(N(c2ccc3c(c2)oc2cc4ccccc4cc23)c2cccc3c2C2(c4ccccc4-c4ccccc42)c2ccccc2-3)cc1 ; Fc1ccc(N(c2cccc3c2C2(c4ccccc4-c4ccccc42)c2ccccc2-3)c2cccc3oc4c5ccccc5ccc4c23)cc1]

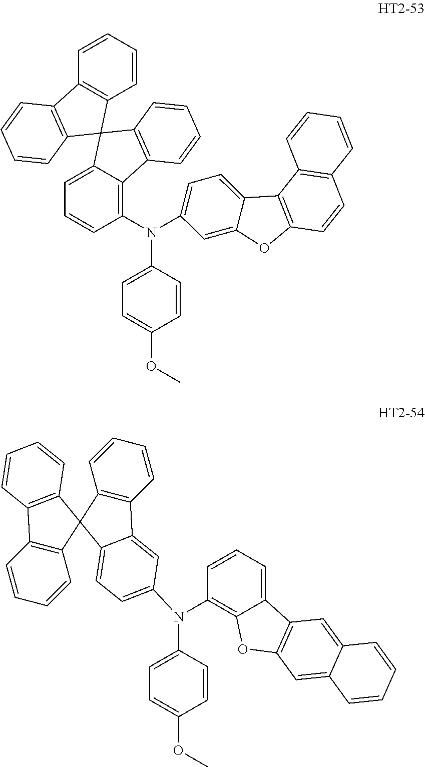 [SMILES: COc1ccc(N(c2ccc3c(c2)oc2ccc4ccccc4c23)c2cccc3c2-c2ccccc2C32c3ccccc3-c3ccccc32)cc1 ; COc1ccc(N(c2ccc3c(c2)-c2ccccc2C32c3ccccc3-c3ccccc32)c2cccc3c2oc2cc4ccccc4cc23)cc1]

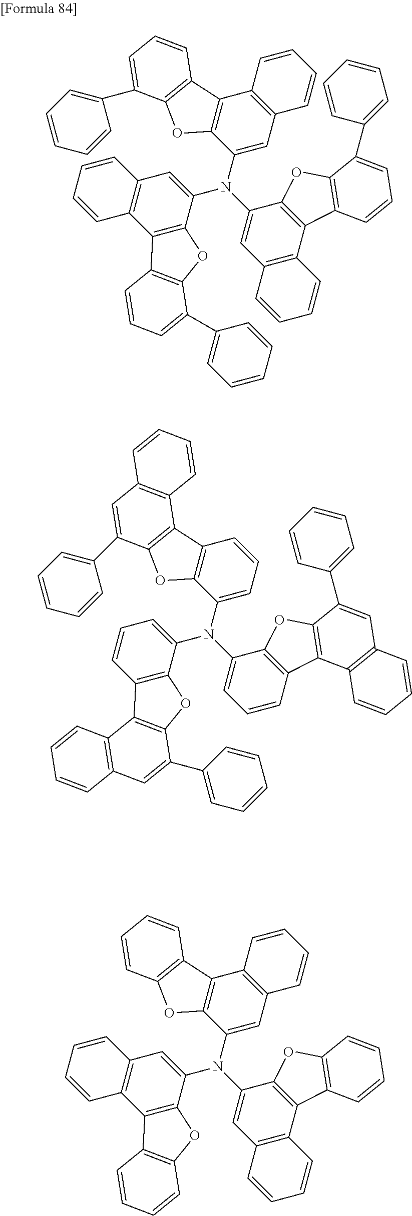 [SMILES: c1ccc(-c2cccc3c2oc2c(N(c4cc5ccccc5c5c4oc4c(-c6ccccc6)cccc45)c4cc5ccccc5c5c4oc4c(-c6ccccc6)cccc45)cc4ccccc4c23)cc1 ; c1ccc(-c2cc3ccccc3c3c2oc2c(N(c4cccc5c4oc4c(-c6ccccc6)cc6ccccc6c45)c4cccc5c4oc4c(-c6ccccc6)cc6ccccc6c45)cccc23)cc1 ; c1ccc2c(c1)cc(N(c1cc3ccccc3c3c1oc1ccccc13)c1cc3ccccc3c3c1oc1ccccc13)c1oc3ccccc3c12]

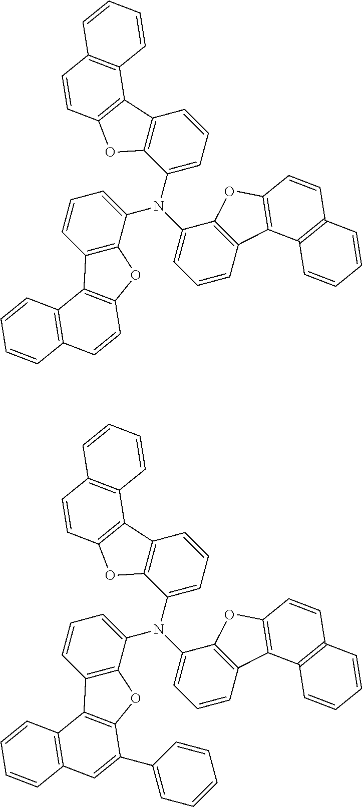 [SMILES: c1ccc2c(c1)ccc1oc3c(N(c4cccc5c4oc4ccc6ccccc6c45)c4cccc5c4oc4ccc6ccccc6c45)cccc3c12 ; c1ccc(-c2cc3ccccc3c3c2oc2c(N(c4cccc5c4oc4ccc6ccccc6c45)c4cccc5c4oc4ccc6ccccc6c45)cccc23)cc1]

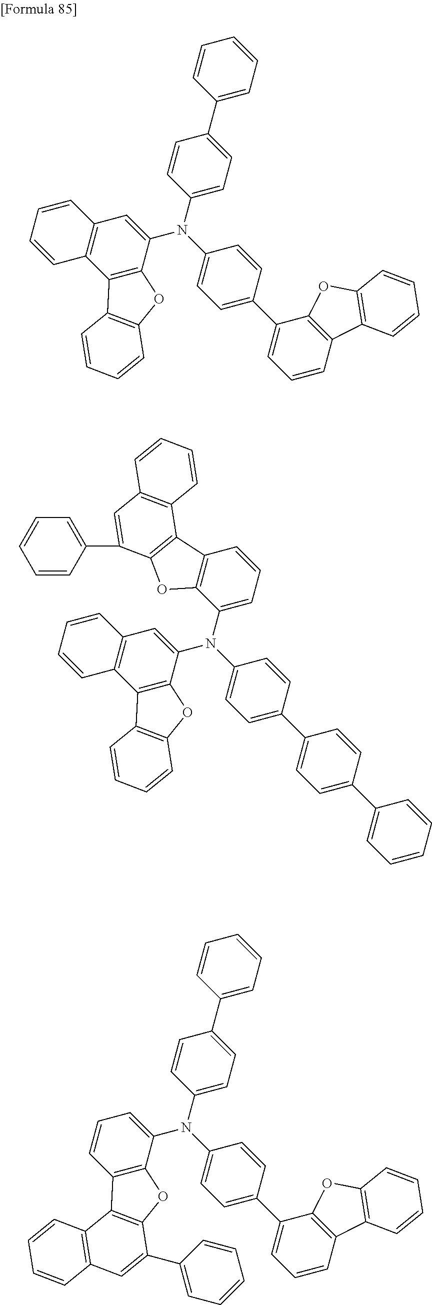 [SMILES: c1ccc(-c2ccc(N(c3ccc(-c4cccc5c4oc4ccccc45)cc3)c3cc4ccccc4c4c3oc3ccccc34)cc2)cc1 ; c1ccc(-c2ccc(-c3ccc(N(c4cccc5c4oc4c(-c6ccccc6)cc6ccccc6c45)c4cc5ccccc5c5c4oc4ccccc45)cc3)cc2)cc1 ; c1ccc(-c2ccc(N(c3ccc(-c4cccc5c4oc4ccccc45)cc3)c3cccc4c3oc3c(-c5ccccc5)cc5ccccc5c34)cc2)cc1]

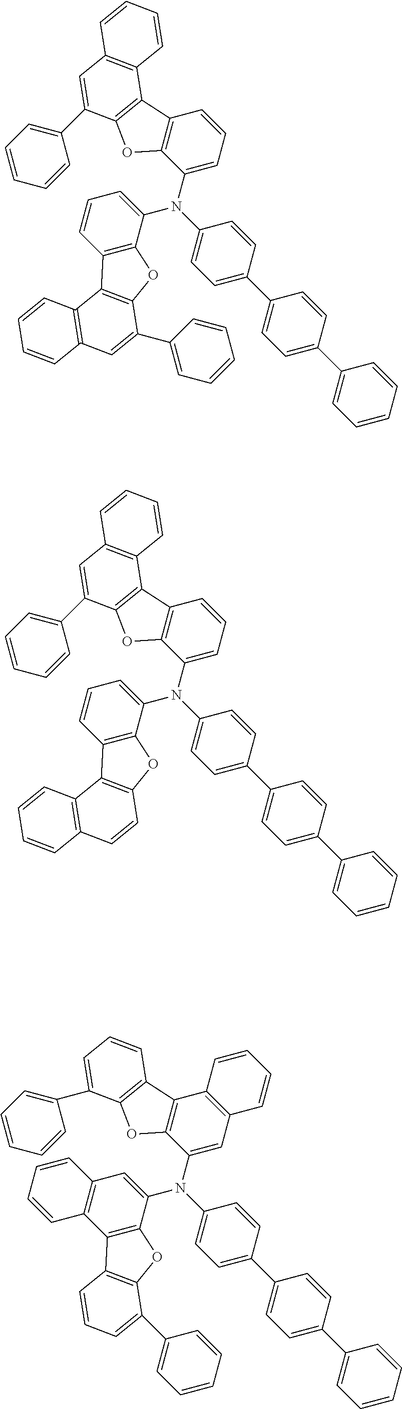 [SMILES: c1ccc(-c2ccc(-c3ccc(N(c4cccc5c4oc4c(-c6ccccc6)cc6ccccc6c45)c4cccc5c4oc4c(-c6ccccc6)cc6ccccc6c45)cc3)cc2)cc1 ; c1ccc(-c2ccc(-c3ccc(N(c4cccc5c4oc4ccc6ccccc6c45)c4cccc5c4oc4c(-c6ccccc6)cc6ccccc6c45)cc3)cc2)cc1 ; c1ccc(-c2ccc(-c3ccc(N(c4cc5ccccc5c5c4oc4c(-c6ccccc6)cccc45)c4cc5ccccc5c5c4oc4c(-c6ccccc6)cccc45)cc3)cc2)cc1]

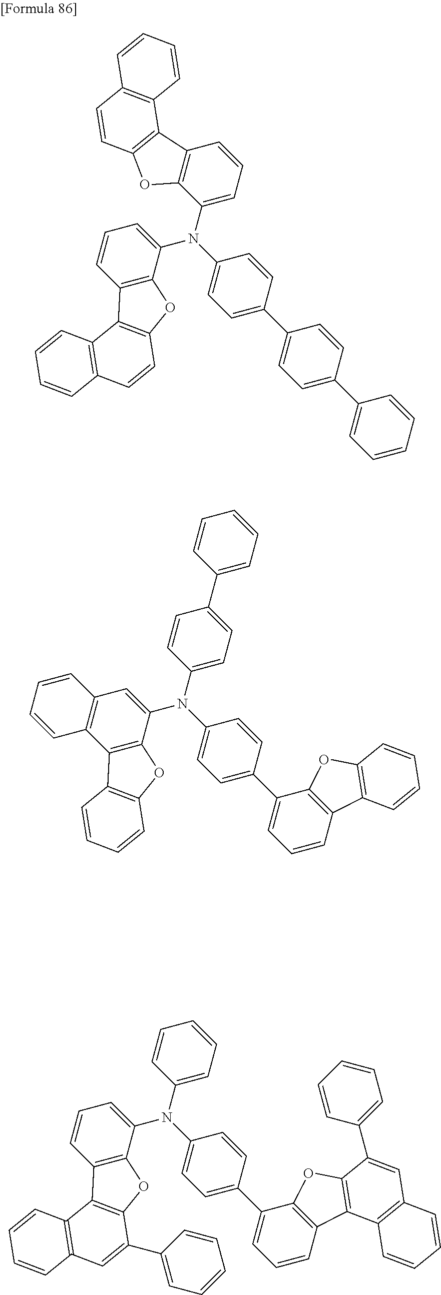 [SMILES: c1ccc(-c2ccc(-c3ccc(N(c4cccc5c4oc4ccc6ccccc6c45)c4cccc5c4oc4ccc6ccccc6c45)cc3)cc2)cc1 ; c1ccc(-c2ccc(N(c3ccc(-c4cccc5c4oc4ccccc45)cc3)c3cc4ccccc4c4c3oc3ccccc34)cc2)cc1 ; c1ccc(-c2cc3ccccc3c3c2oc2c(-c4ccc(N(c5ccccc5)c5cccc6c5oc5c(-c7ccccc7)cc7ccccc7c56)cc4)cccc23)cc1]

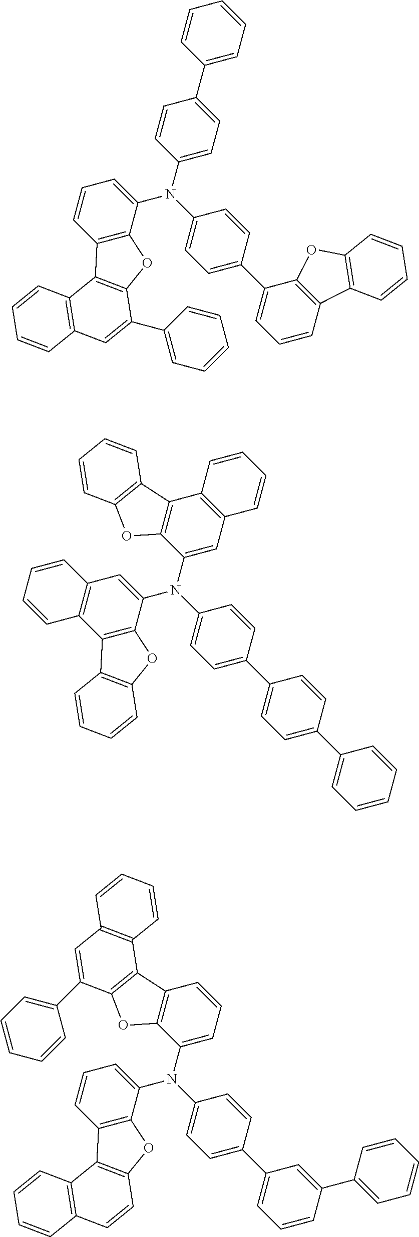 [SMILES: c1ccc(-c2ccc(N(c3ccc(-c4cccc5c4oc4ccccc45)cc3)c3cccc4c3oc3c(-c5ccccc5)cc5ccccc5c34)cc2)cc1 ; c1ccc(-c2ccc(-c3ccc(N(c4cc5ccccc5c5c4oc4ccccc45)c4cc5ccccc5c5c4oc4ccccc45)cc3)cc2)cc1 ; c1ccc(-c2cccc(-c3ccc(N(c4cccc5c4oc4ccc6ccccc6c45)c4cccc5c4oc4c(-c6ccccc6)cc6ccccc6c45)cc3)c2)cc1]

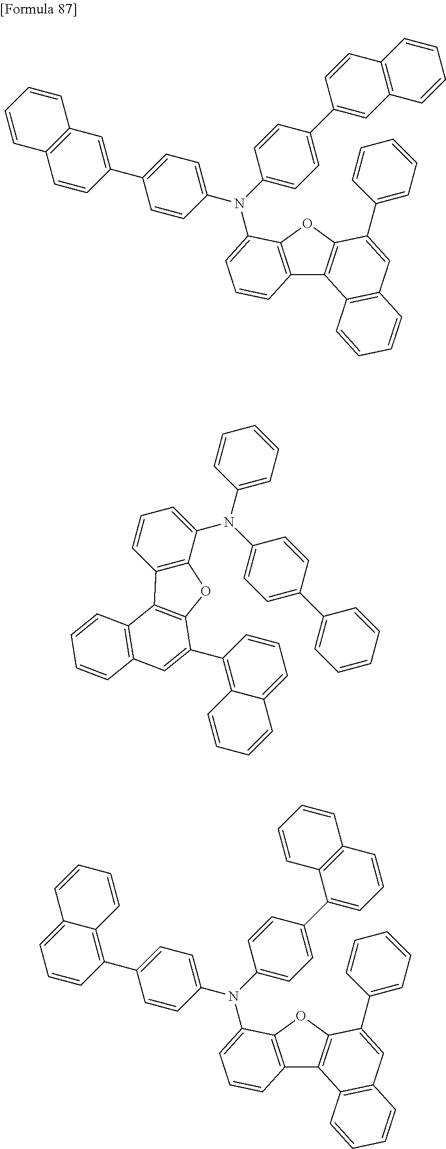 [SMILES: c1ccc(-c2cc3ccccc3c3c2oc2c(N(c4ccc(-c5ccc6ccccc6c5)cc4)c4ccc(-c5ccc6ccccc6c5)cc4)cccc23)cc1 ; c1ccc(-c2ccc(N(c3ccccc3)c3cccc4c3oc3c(-c5cccc6ccccc56)cc5ccccc5c34)cc2)cc1 ; c1ccc(-c2cc3ccccc3c3c2oc2c(N(c4ccc(-c5cccc6ccccc56)cc4)c4ccc(-c5cccc6ccccc56)cc4)cccc23)cc1]

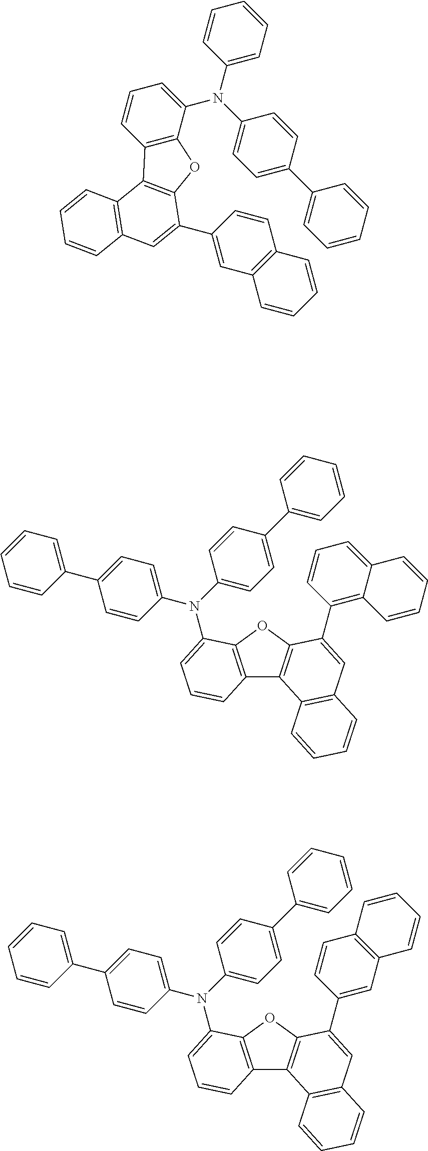 [SMILES: c1ccc(-c2ccc(N(c3ccccc3)c3cccc4c3oc3c(-c5ccc6ccccc6c5)cc5ccccc5c34)cc2)cc1 ; c1ccc(-c2ccc(N(c3ccc(-c4ccccc4)cc3)c3cccc4c3oc3c(-c5cccc6ccccc56)cc5ccccc5c34)cc2)cc1 ; c1ccc(-c2ccc(N(c3ccc(-c4ccccc4)cc3)c3cccc4c3oc3c(-c5ccc6ccccc6c5)cc5ccccc5c34)cc2)cc1]

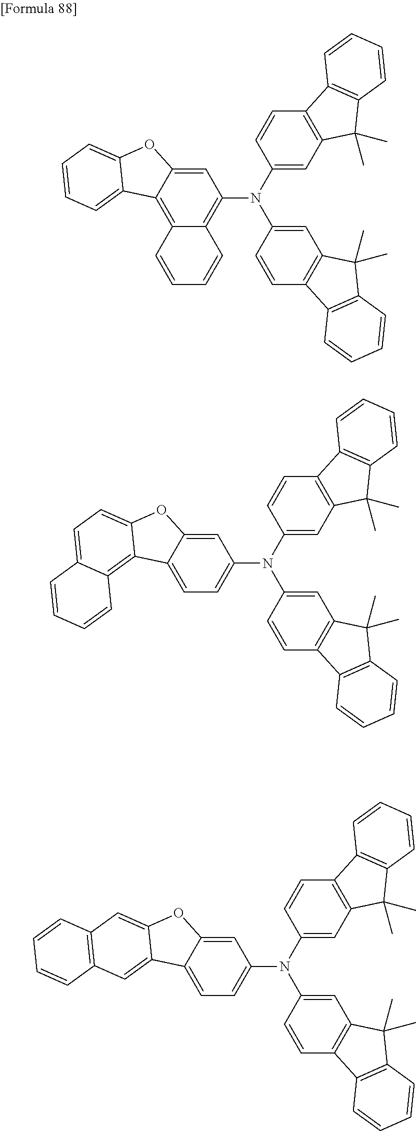

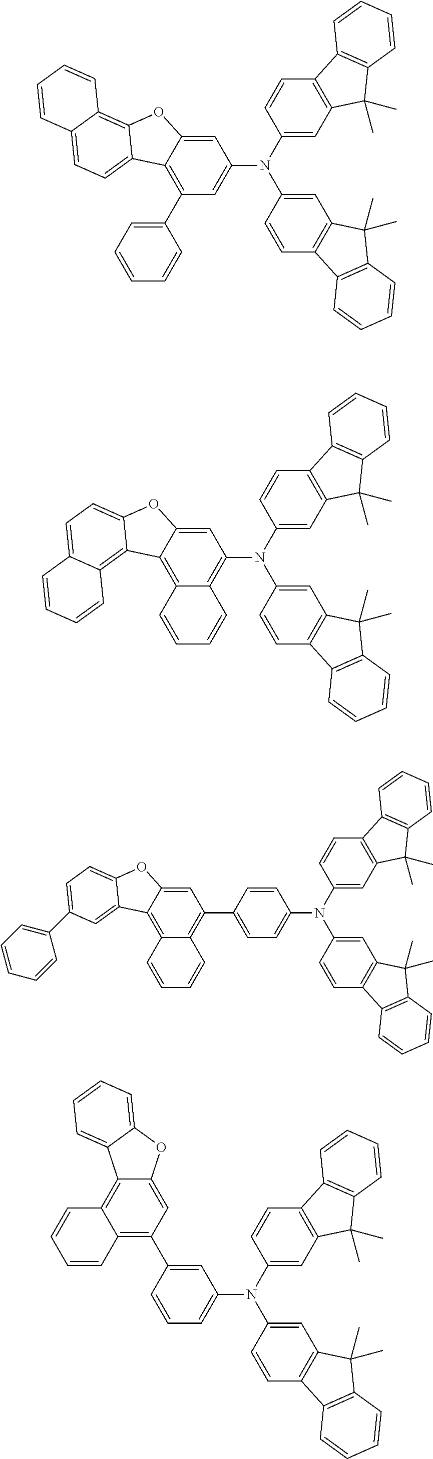 [SMILES: CC1(C)c2ccccc2-c2ccc(N(c3ccc4c(c3)C(C)(C)c3ccccc3-4)c3cc(-c4ccccc4)c4c(c3)oc3c5ccccc5ccc34)cc21 ; CC1(C)c2ccccc2-c2ccc(N(c3ccc4c(c3)C(C)(C)c3ccccc3-4)c3cc4oc5ccc6ccccc6c5c4c4ccccc34)cc21 ; CC1(C)c2ccccc2-c2ccc(N(c3ccc(-c4cc5oc6ccc(-c7ccccc7)cc6c5c5ccccc45)cc3)c3ccc4c(c3)C(C)(C)c3ccccc3-4)cc21 ; CC1(C)c2ccccc2-c2ccc(N(c3cccc(-c4cc5oc6ccccc6c5c5ccccc45)c3)c3ccc4c(c3)C(C)(C)c3ccccc3-4)cc21]

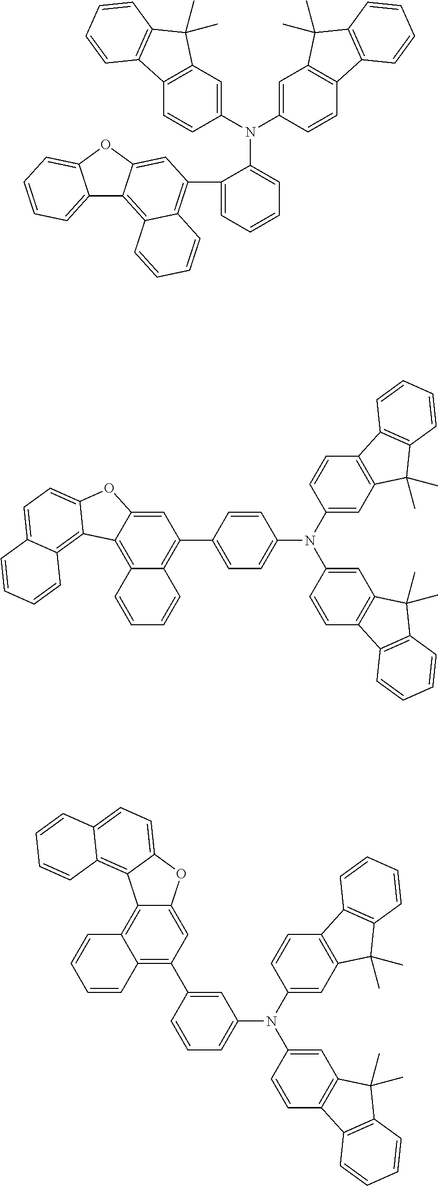 [SMILES: CC1(C)c2ccccc2-c2ccc(N(c3ccc4c(c3)C(C)(C)c3ccccc3-4)c3ccccc3-c3cc4oc5ccccc5c4c4ccccc34)cc21 ; CC1(C)c2ccccc2-c2ccc(N(c3ccc(-c4cc5oc6ccc7ccccc7c6c5c5ccccc45)cc3)c3ccc4c(c3)C(C)(C)c3ccccc3-4)cc21 ; CC1(C)c2ccccc2-c2ccc(N(c3cccc(-c4cc5oc6ccc7ccccc7c6c5c5ccccc45)c3)c3ccc4c(c3)C(C)(C)c3ccccc3-4)cc21]

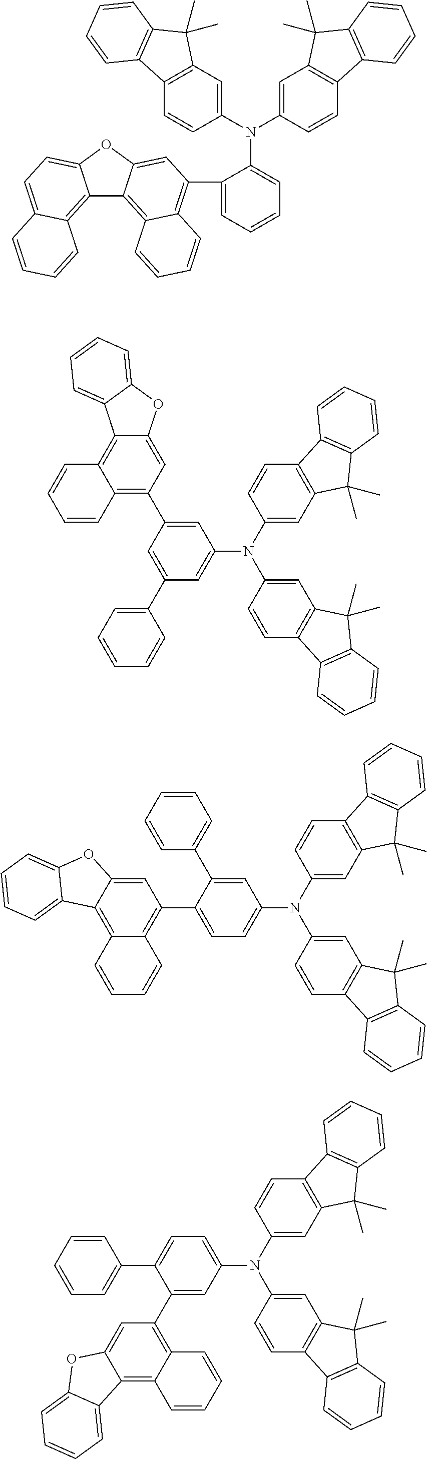

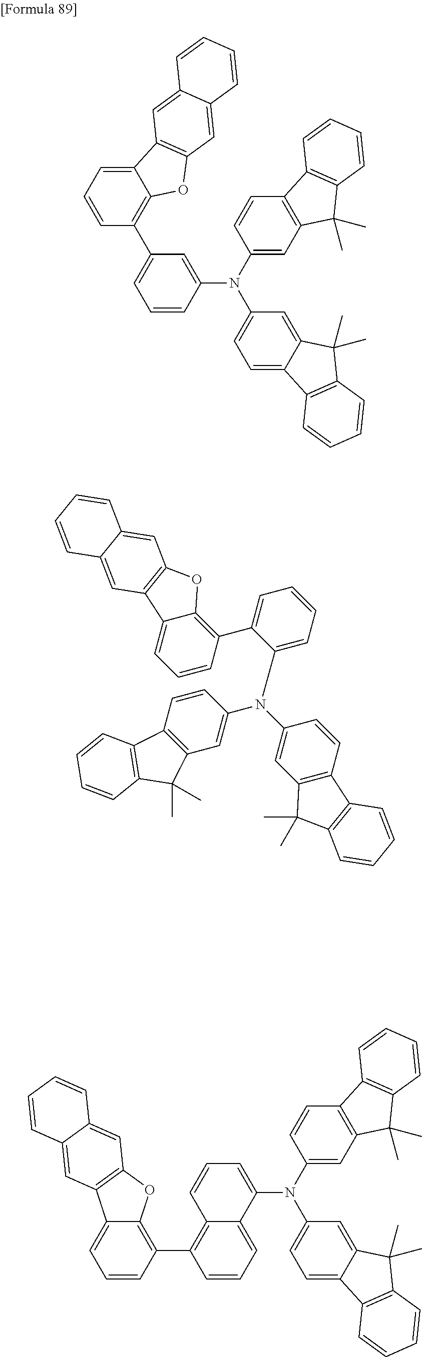 [SMILES: CC1(C)c2ccccc2-c2ccc(N(c3cccc(-c4cccc5c4oc4cc6ccccc6cc45)c3)c3ccc4c(c3)C(C)(C)c3ccccc3-4)cc21 ; CC1(C)c2ccccc2-c2ccc(N(c3ccc4c(c3)C(C)(C)c3ccccc3-4)c3ccccc3-c3cccc4c3oc3cc5ccccc5cc34)cc21 ; CC1(C)c2ccccc2-c2ccc(N(c3ccc4c(c3)C(C)(C)c3ccccc3-4)c3cccc4c(-c5cccc6c5oc5cc7ccccc7cc56)cccc34)cc21]

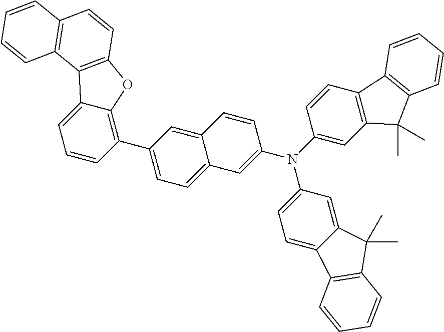 [SMILES: CC1(C)c2ccccc2-c2ccc(N(c3ccc4c(c3)C(C)(C)c3ccccc3-4)c3ccc4cc(-c5cccc6c5oc5ccc7ccccc7c56)ccc4c3)cc21]

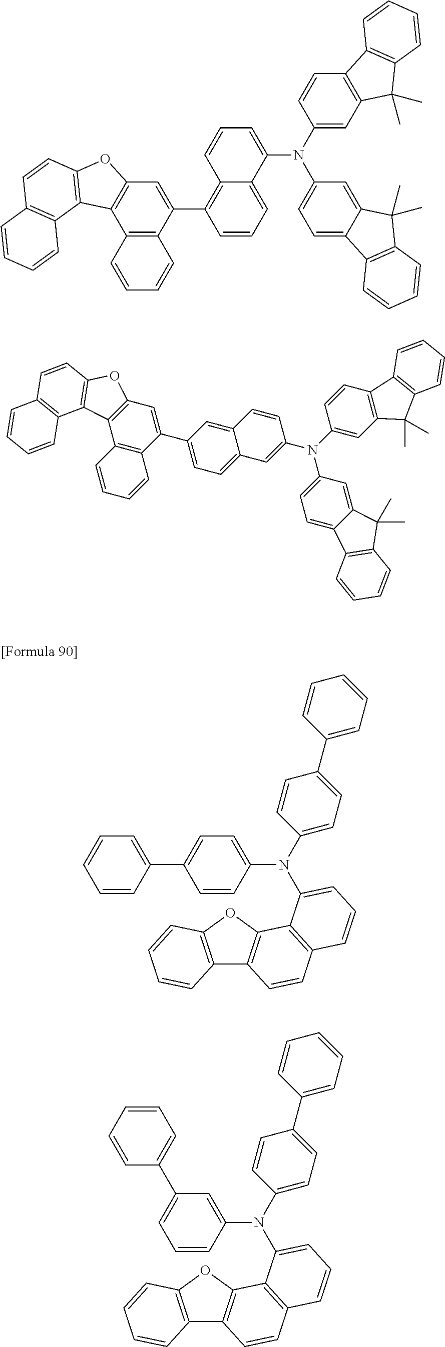

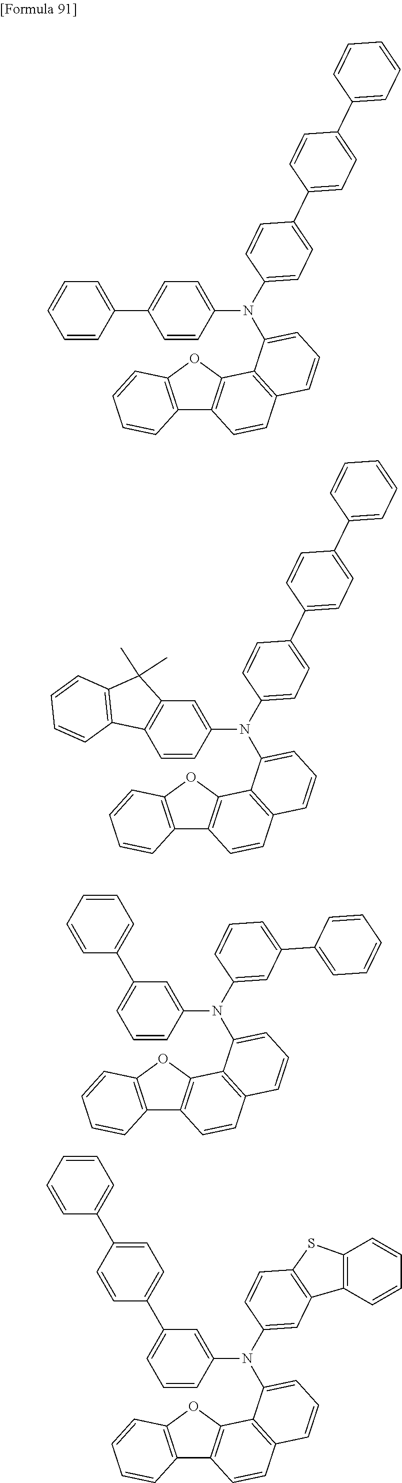

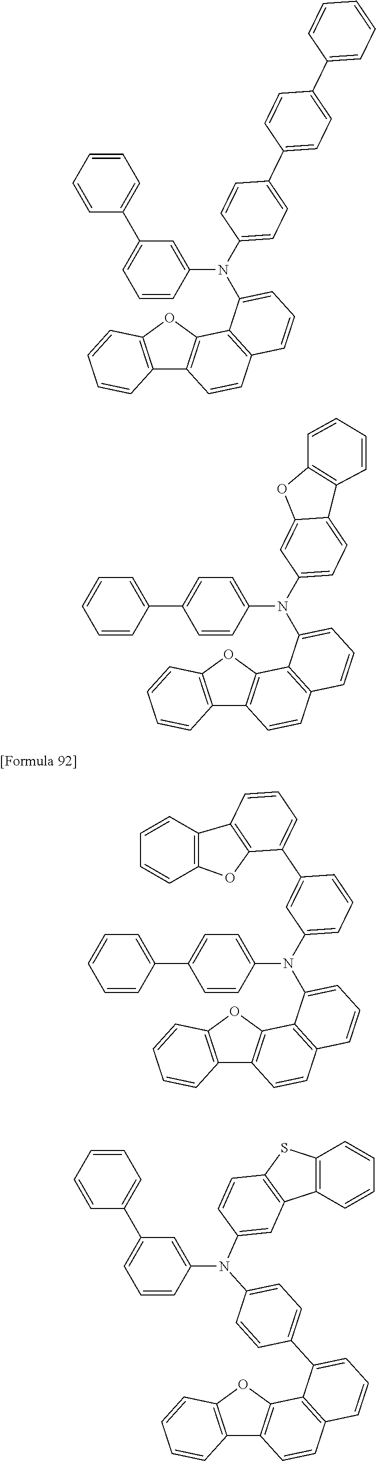 [SMILES: c1ccc(-c2ccc(-c3ccc(N(c4cccc(-c5ccccc5)c4)c4cccc5ccc6c7ccccc7oc6c45)cc3)cc2)cc1 ; c1ccc(-c2ccc(N(c3ccc4c(c3)oc3ccccc34)c3cccc4ccc5c6ccccc6oc5c34)cc2)cc1 ; c1ccc(-c2ccc(N(c3cccc(-c4cccc5c4oc4ccccc45)c3)c3cccc4ccc5c6ccccc6oc5c34)cc2)cc1 ; c1ccc(-c2cccc(N(c3ccc(-c4cccc5ccc6c7ccccc7oc6c45)cc3)c3ccc4sc5ccccc5c4c3)c2)cc1]

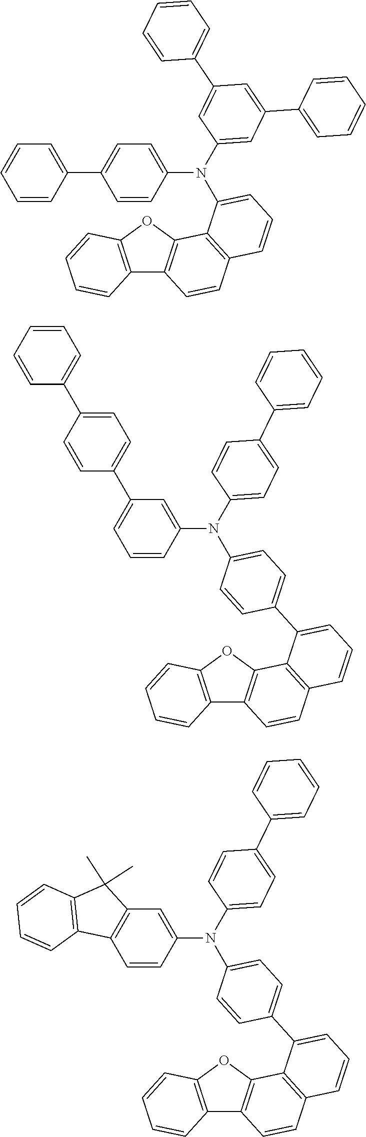

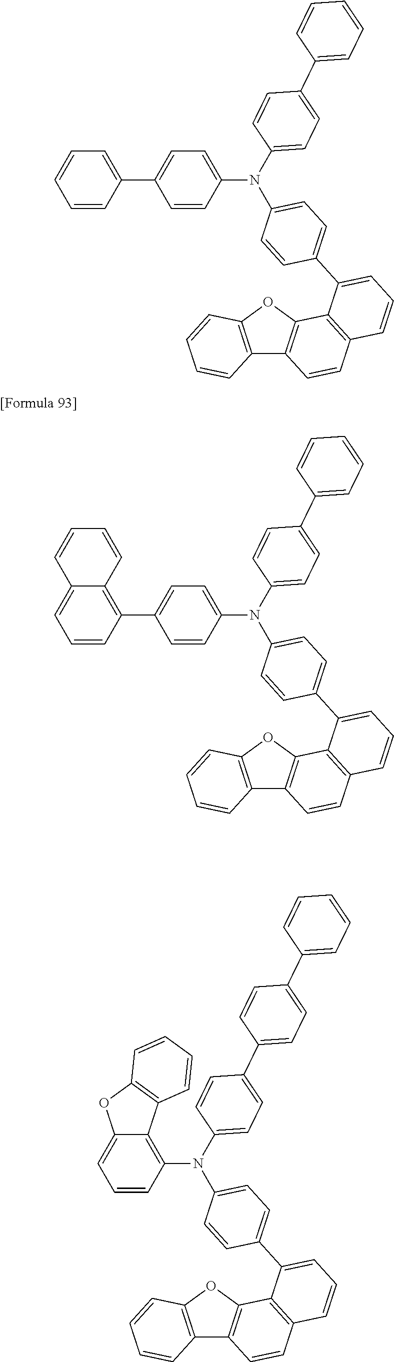 [SMILES: c1ccc(-c2ccc(N(c3ccc(-c4ccccc4)cc3)c3ccc(-c4cccc5ccc6c7ccccc7oc6c45)cc3)cc2)cc1 ; c1ccc(-c2ccc(N(c3ccc(-c4cccc5ccccc45)cc3)c3ccc(-c4cccc5ccc6c7ccccc7oc6c45)cc3)cc2)cc1 ; c1ccc(-c2ccc(-c3ccc(N(c4ccc(-c5cccc6ccc7c8ccccc8oc7c56)cc4)c4cccc5oc6ccccc6c45)cc3)cc2)cc1]

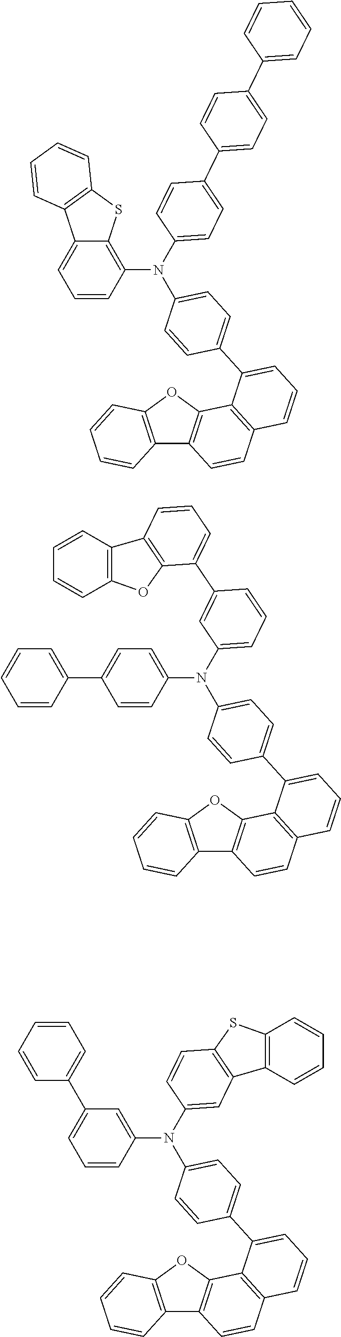

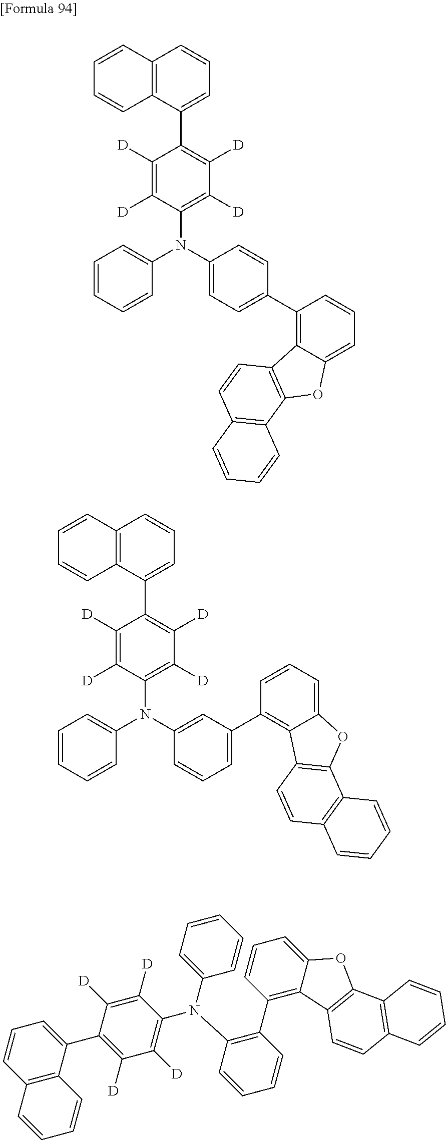 [SMILES: [2H]c1c([2H])c(N(c2ccccc2)c2ccc(-c3cccc4oc5c6ccccc6ccc5c34)cc2)c([2H])c([2H])c1-c1cccc2ccccc12 ; [2H]c1c([2H])c(N(c2ccccc2)c2cccc(-c3cccc4oc5c6ccccc6ccc5c34)c2)c([2H])c([2H])c1-c1cccc2ccccc12 ; [2H]c1c([2H])c(N(c2ccccc2)c2ccccc2-c2cccc3oc4c5ccccc5ccc4c23)c([2H])c([2H])c1-c1cccc2ccccc12]

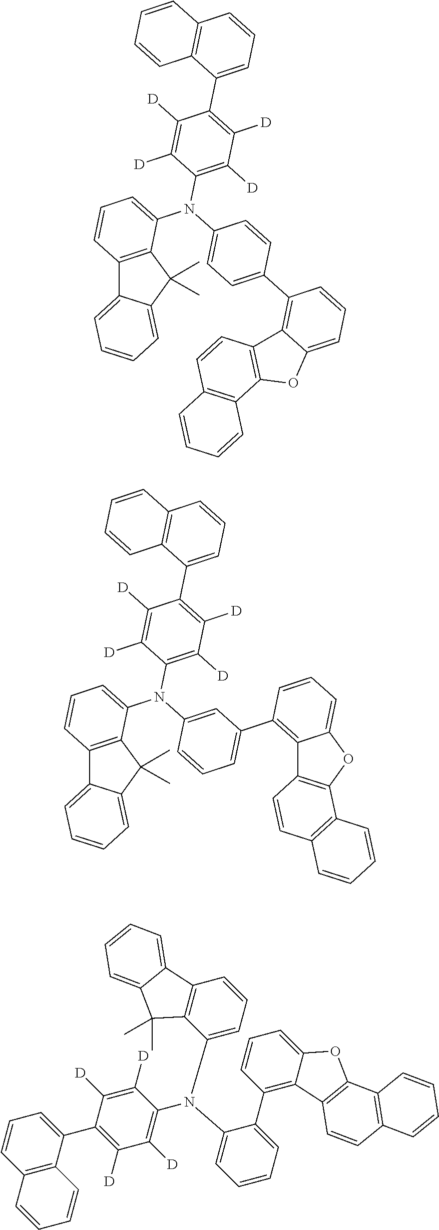 [SMILES: [2H]c1c([2H])c(N(c2ccc(-c3cccc4oc5c6ccccc6ccc5c34)cc2)c2cccc3c2C(C)(C)c2ccccc2-3)c([2H])c([2H])c1-c1cccc2ccccc12 ; [2H]c1c([2H])c(N(c2cccc(-c3cccc4oc5c6ccccc6ccc5c34)c2)c2cccc3c2C(C)(C)c2ccccc2-3)c([2H])c([2H])c1-c1cccc2ccccc12 ; [2H]c1c([2H])c(N(c2ccccc2-c2cccc3oc4c5ccccc5ccc4c23)c2cccc3c2C(C)(C)c2ccccc2-3)c([2H])c([2H])c1-c1cccc2ccccc12]

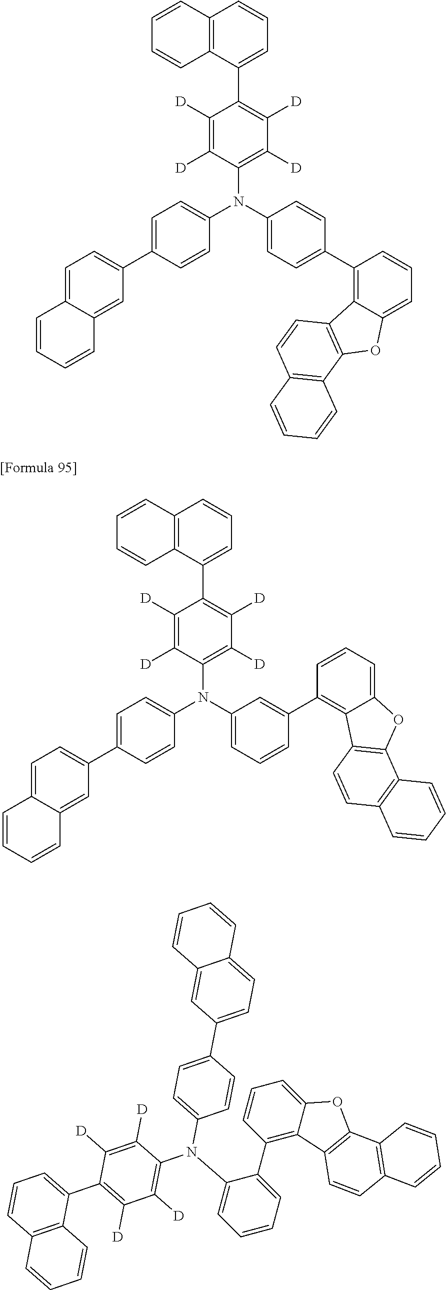 [SMILES: [2H]c1c([2H])c(N(c2ccc(-c3ccc4ccccc4c3)cc2)c2ccc(-c3cccc4oc5c6ccccc6ccc5c34)cc2)c([2H])c([2H])c1-c1cccc2ccccc12 ; [2H]c1c([2H])c(N(c2ccc(-c3ccc4ccccc4c3)cc2)c2cccc(-c3cccc4oc5c6ccccc6ccc5c34)c2)c([2H])c([2H])c1-c1cccc2ccccc12 ; [2H]c1c([2H])c(N(c2ccc(-c3ccc4ccccc4c3)cc2)c2ccccc2-c2cccc3oc4c5ccccc5ccc4c23)c([2H])c([2H])c1-c1cccc2ccccc12]

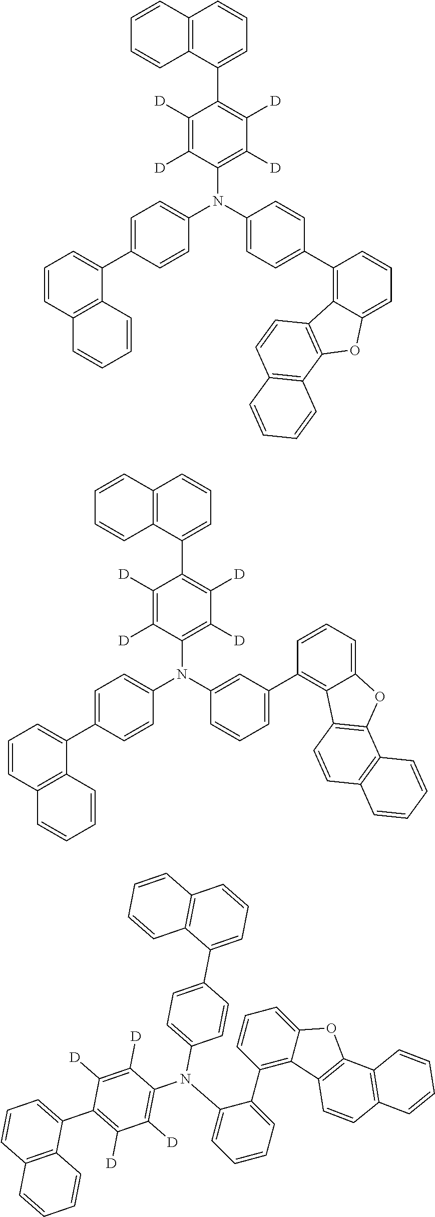 [SMILES: [2H]c1c([2H])c(N(c2ccc(-c3cccc4ccccc34)cc2)c2ccc(-c3cccc4oc5c6ccccc6ccc5c34)cc2)c([2H])c([2H])c1-c1cccc2ccccc12 ; [2H]c1c([2H])c(N(c2ccc(-c3cccc4ccccc34)cc2)c2cccc(-c3cccc4oc5c6ccccc6ccc5c34)c2)c([2H])c([2H])c1-c1cccc2ccccc12 ; [2H]c1c([2H])c(N(c2ccc(-c3cccc4ccccc34)cc2)c2ccccc2-c2cccc3oc4c5ccccc5ccc4c23)c([2H])c([2H])c1-c1cccc2ccccc12]

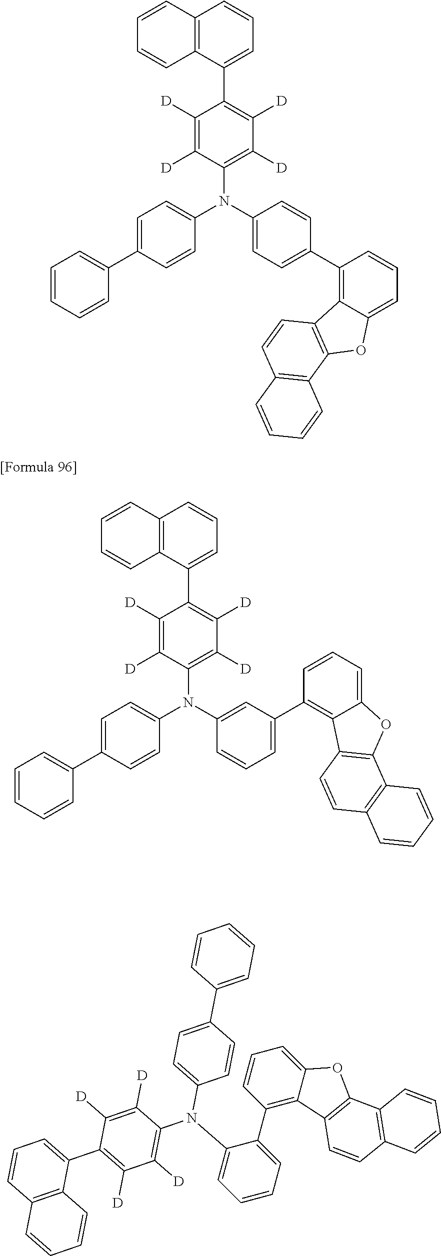 [SMILES: [2H]c1c([2H])c(N(c2ccc(-c3ccccc3)cc2)c2ccc(-c3cccc4oc5c6ccccc6ccc5c34)cc2)c([2H])c([2H])c1-c1cccc2ccccc12 ; [2H]c1c([2H])c(N(c2ccc(-c3ccccc3)cc2)c2cccc(-c3cccc4oc5c6ccccc6ccc5c34)c2)c([2H])c([2H])c1-c1cccc2ccccc12 ; [2H]c1c([2H])c(N(c2ccc(-c3ccccc3)cc2)c2ccccc2-c2cccc3oc4c5ccccc5ccc4c23)c([2H])c([2H])c1-c1cccc2ccccc12]

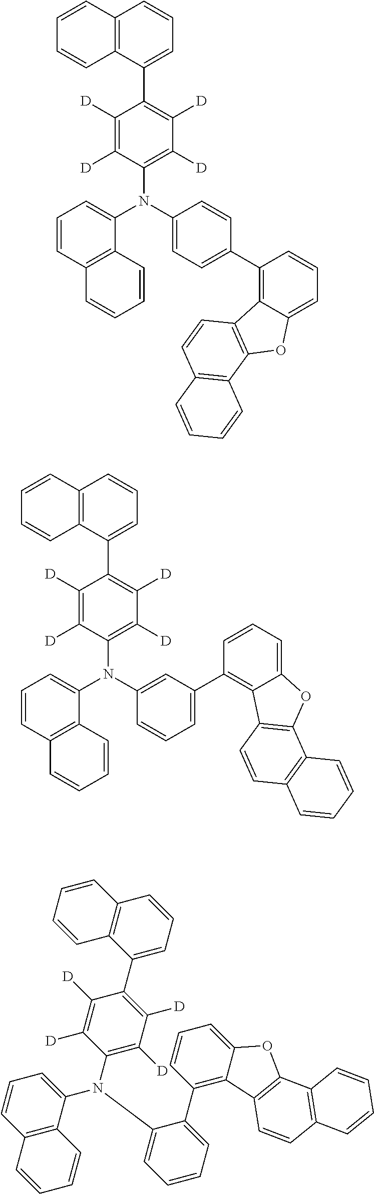 [SMILES: [2H]c1c([2H])c(N(c2ccc(-c3cccc4oc5c6ccccc6ccc5c34)cc2)c2cccc3ccccc23)c([2H])c([2H])c1-c1cccc2ccccc12 ; [2H]c1c([2H])c(N(c2cccc(-c3cccc4oc5c6ccccc6ccc5c34)c2)c2cccc3ccccc23)c([2H])c([2H])c1-c1cccc2ccccc12 ; [2H]c1c([2H])c(N(c2ccccc2-c2cccc3oc4c5ccccc5ccc4c23)c2cccc3ccccc23)c([2H])c([2H])c1-c1cccc2ccccc12]

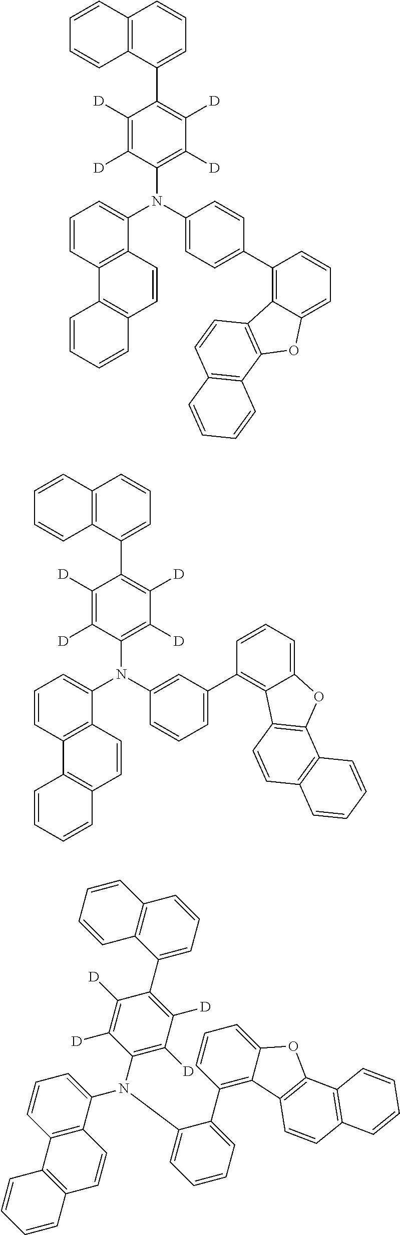 [SMILES: [2H]c1c([2H])c(N(c2ccc(-c3cccc4oc5c6ccccc6ccc5c34)cc2)c2cccc3c2ccc2ccccc23)c([2H])c([2H])c1-c1cccc2ccccc12 ; [2H]c1c([2H])c(N(c2cccc(-c3cccc4oc5c6ccccc6ccc5c34)c2)c2cccc3c2ccc2ccccc23)c([2H])c([2H])c1-c1cccc2ccccc12 ; [2H]c1c([2H])c(N(c2ccccc2-c2cccc3oc4c5ccccc5ccc4c23)c2cccc3c2ccc2ccccc23)c([2H])c([2H])c1-c1cccc2ccccc12]

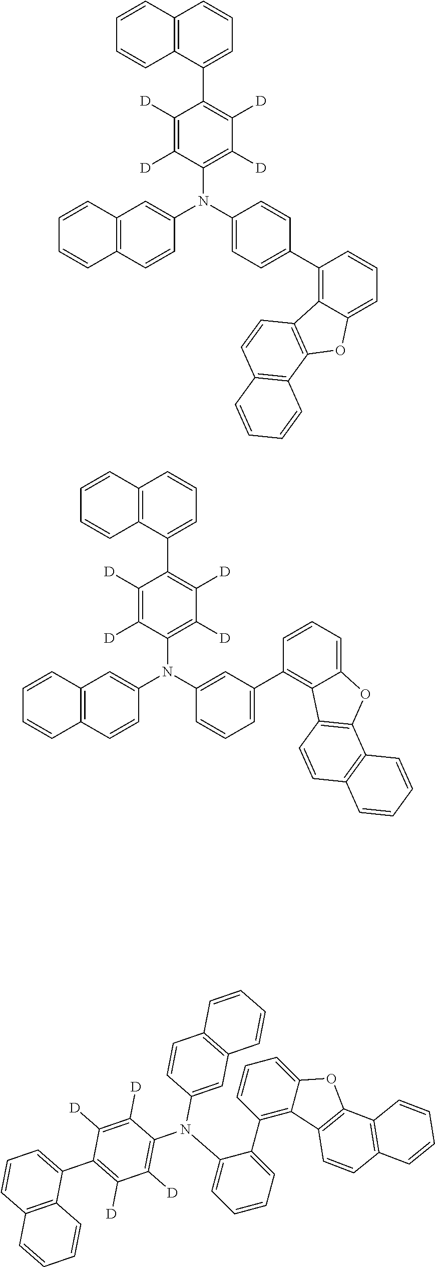 [SMILES: [2H]c1c([2H])c(N(c2ccc(-c3cccc4oc5c6ccccc6ccc5c34)cc2)c2ccc3ccccc3c2)c([2H])c([2H])c1-c1cccc2ccccc12 ; [2H]c1c([2H])c(N(c2cccc(-c3cccc4oc5c6ccccc6ccc5c34)c2)c2ccc3ccccc3c2)c([2H])c([2H])c1-c1cccc2ccccc12 ; [2H]c1c([2H])c(N(c2ccc3ccccc3c2)c2ccccc2-c2cccc3oc4c5ccccc5ccc4c23)c([2H])c([2H])c1-c1cccc2ccccc12]

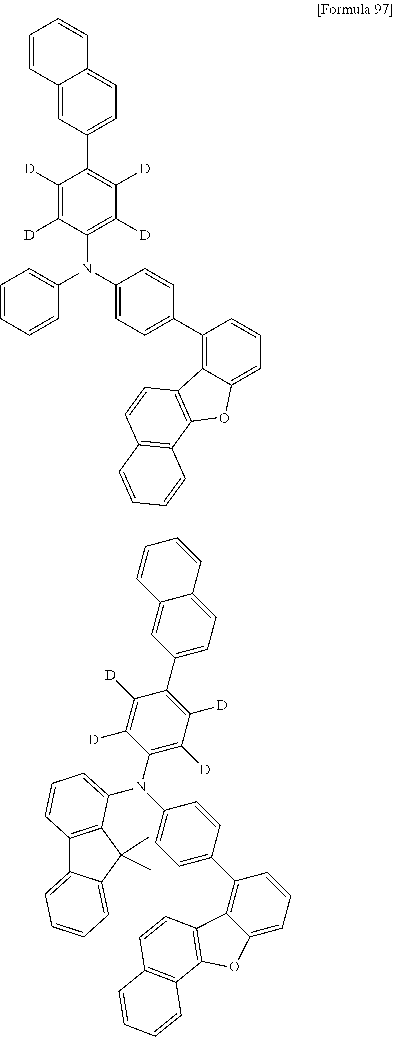

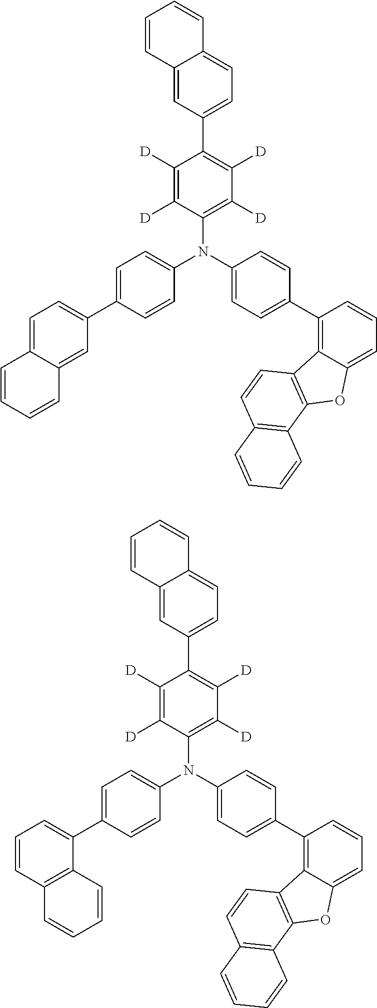 [SMILES: [2H]c1c([2H])c(N(c2ccc(-c3ccc4ccccc4c3)cc2)c2ccc(-c3cccc4oc5c6ccccc6ccc5c34)cc2)c([2H])c([2H])c1-c1ccc2ccccc2c1 ; [2H]c1c([2H])c(N(c2ccc(-c3cccc4ccccc34)cc2)c2ccc(-c3cccc4oc5c6ccccc6ccc5c34)cc2)c([2H])c([2H])c1-c1ccc2ccccc2c1]

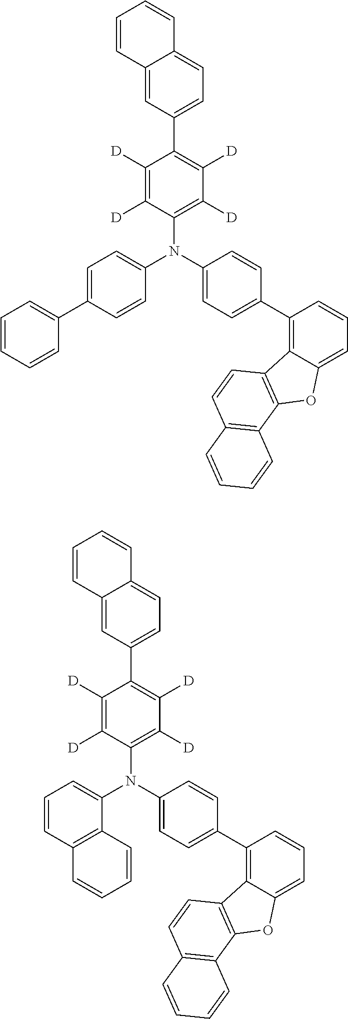 [SMILES: [2H]c1c([2H])c(N(c2ccc(-c3ccccc3)cc2)c2ccc(-c3cccc4oc5c6ccccc6ccc5c34)cc2)c([2H])c([2H])c1-c1ccc2ccccc2c1 ; [2H]c1c([2H])c(N(c2ccc(-c3cccc4oc5c6ccccc6ccc5c34)cc2)c2cccc3ccccc23)c([2H])c([2H])c1-c1ccc2ccccc2c1]

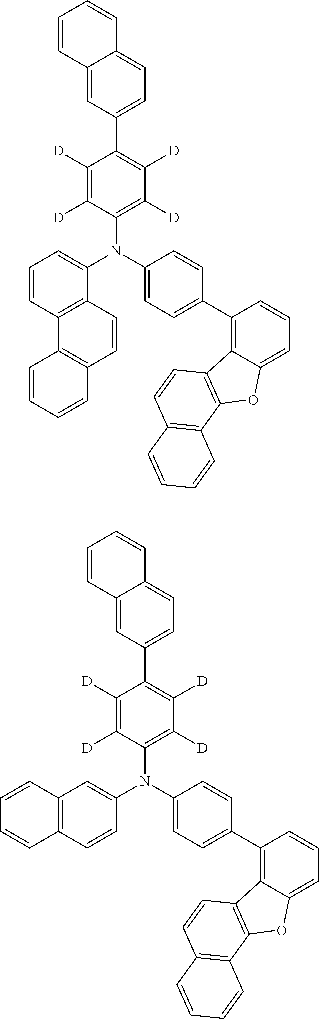 [SMILES: [2H]c1c([2H])c(N(c2ccc(-c3cccc4oc5c6ccccc6ccc5c34)cc2)c2cccc3c2ccc2ccccc23)c([2H])c([2H])c1-c1ccc2ccccc2c1 ; [2H]c1c([2H])c(N(c2ccc(-c3cccc4oc5c6ccccc6ccc5c34)cc2)c2ccc3ccccc3c2)c([2H])c([2H])c1-c1ccc2ccccc2c1]

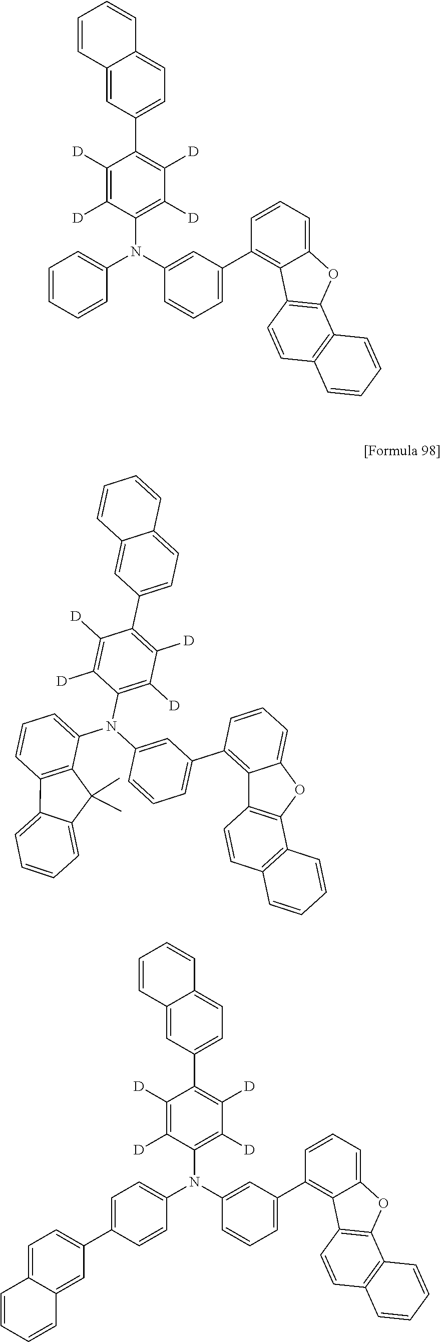 [SMILES: [2H]c1c([2H])c(N(c2ccccc2)c2cccc(-c3cccc4oc5c6ccccc6ccc5c34)c2)c([2H])c([2H])c1-c1ccc2ccccc2c1 ; [2H]c1c([2H])c(N(c2cccc(-c3cccc4oc5c6ccccc6ccc5c34)c2)c2cccc3c2C(C)(C)c2ccccc2-3)c([2H])c([2H])c1-c1ccc2ccccc2c1 ; [2H]c1c([2H])c(N(c2ccc(-c3ccc4ccccc4c3)cc2)c2cccc(-c3cccc4oc5c6ccccc6ccc5c34)c2)c([2H])c([2H])c1-c1ccc2ccccc2c1]

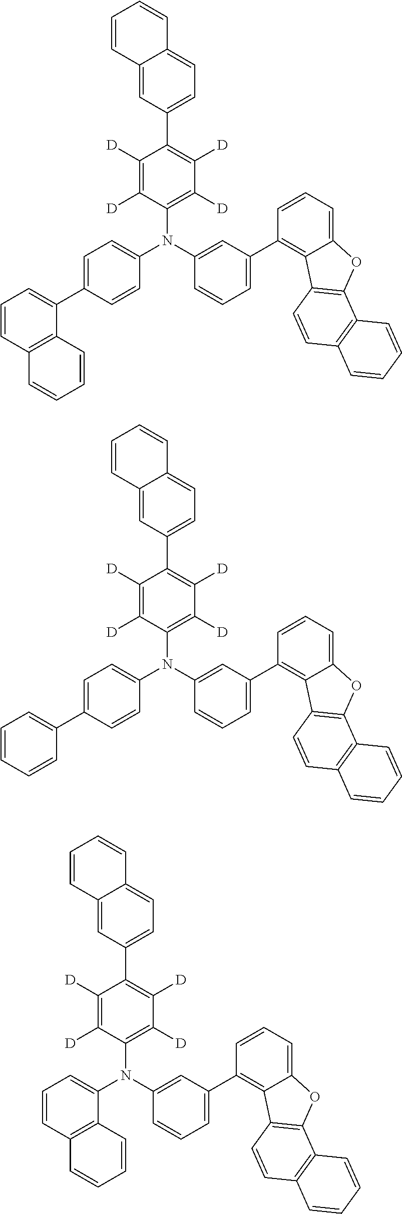 [SMILES: [2H]c1c([2H])c(N(c2ccc(-c3cccc4ccccc34)cc2)c2cccc(-c3cccc4oc5c6ccccc6ccc5c34)c2)c([2H])c([2H])c1-c1ccc2ccccc2c1 ; [2H]c1c([2H])c(N(c2ccc(-c3ccccc3)cc2)c2cccc(-c3cccc4oc5c6ccccc6ccc5c34)c2)c([2H])c([2H])c1-c1ccc2ccccc2c1 ; [2H]c1c([2H])c(N(c2cccc(-c3cccc4oc5c6ccccc6ccc5c34)c2)c2cccc3ccccc23)c([2H])c([2H])c1-c1ccc2ccccc2c1]

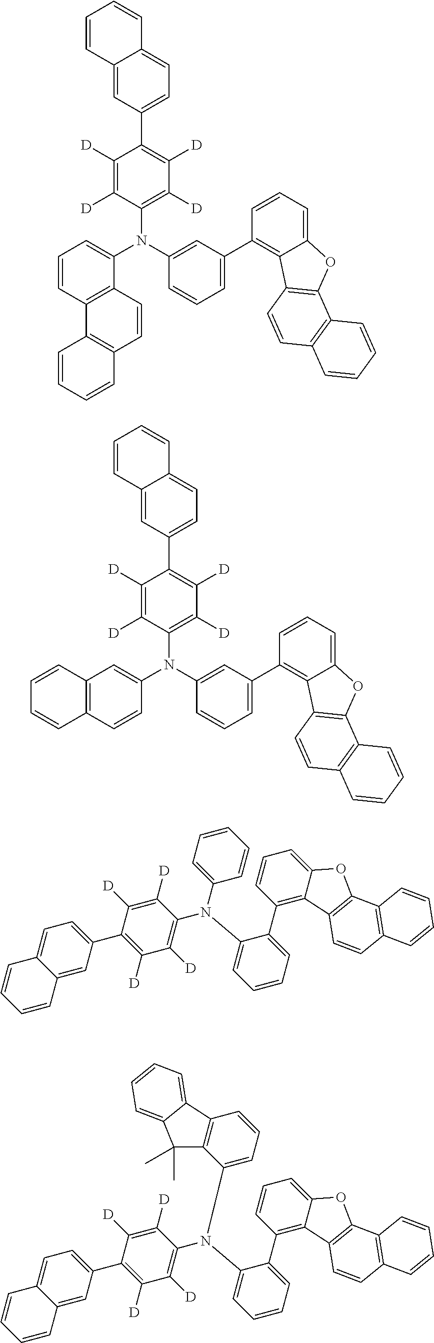 [SMILES: [2H]c1c([2H])c(N(c2cccc(-c3cccc4oc5c6ccccc6ccc5c34)c2)c2cccc3c2ccc2ccccc23)c([2H])c([2H])c1-c1ccc2ccccc2c1 ; [2H]c1c([2H])c(N(c2cccc(-c3cccc4oc5c6ccccc6ccc5c34)c2)c2ccc3ccccc3c2)c([2H])c([2H])c1-c1ccc2ccccc2c1 ; [2H]c1c([2H])c(N(c2ccccc2)c2ccccc2-c2cccc3oc4c5ccccc5ccc4c23)c([2H])c([2H])c1-c1ccc2ccccc2c1 ; [2H]c1c([2H])c(N(c2ccccc2-c2cccc3oc4c5ccccc5ccc4c23)c2cccc3c2C(C)(C)c2ccccc2-3)c([2H])c([2H])c1-c1ccc2ccccc2c1]

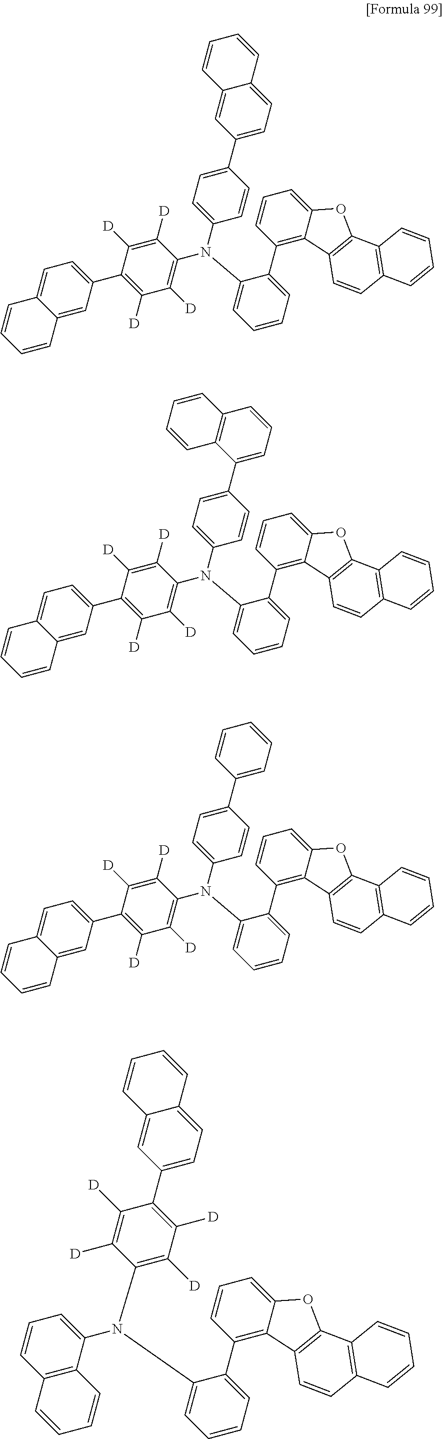 [SMILES: [2H]c1c([2H])c(N(c2ccc(-c3ccc4ccccc4c3)cc2)c2ccccc2-c2cccc3oc4c5ccccc5ccc4c23)c([2H])c([2H])c1-c1ccc2ccccc2c1 ; [2H]c1c([2H])c(N(c2ccc(-c3cccc4ccccc34)cc2)c2ccccc2-c2cccc3oc4c5ccccc5ccc4c23)c([2H])c([2H])c1-c1ccc2ccccc2c1 ; [2H]c1c([2H])c(N(c2ccc(-c3ccccc3)cc2)c2ccccc2-c2cccc3oc4c5ccccc5ccc4c23)c([2H])c([2H])c1-c1ccc2ccccc2c1 ; [2H]c1c([2H])c(N(c2ccccc2-c2cccc3oc4c5ccccc5ccc4c23)c2cccc3ccccc23)c([2H])c([2H])c1-c1ccc2ccccc2c1]

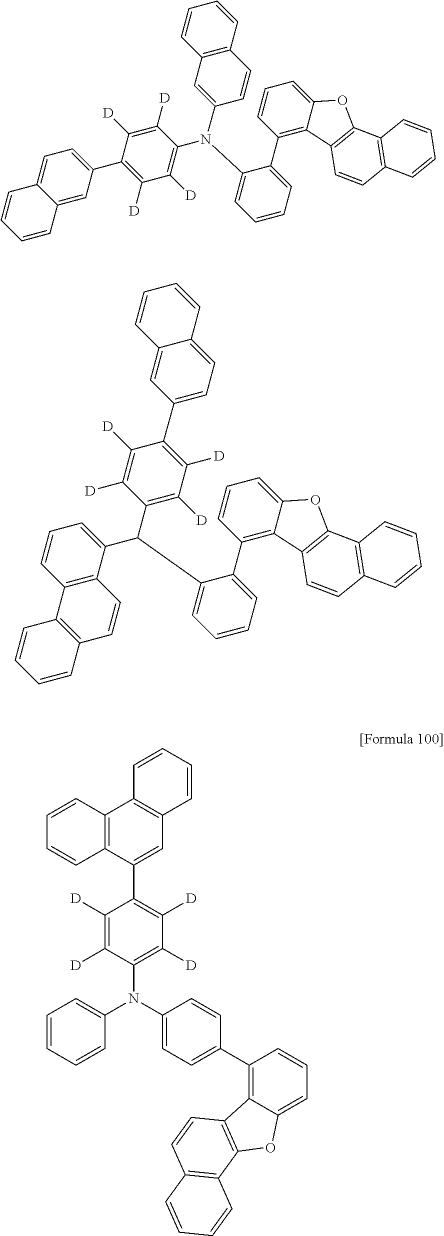 [SMILES: [2H]c1c([2H])c(N(c2ccc3ccccc3c2)c2ccccc2-c2cccc3oc4c5ccccc5ccc4c23)c([2H])c([2H])c1-c1ccc2ccccc2c1 ; [2H]c1c([2H])c(C(c2ccccc2-c2cccc3oc4c5ccccc5ccc4c23)c2cccc3c2ccc2ccccc23)c([2H])c([2H])c1-c1ccc2ccccc2c1 ; [2H]c1c([2H])c(N(c2ccccc2)c2ccc(-c3cccc4oc5c6ccccc6ccc5c34)cc2)c([2H])c([2H])c1-c1cc2ccccc2c2ccccc12]

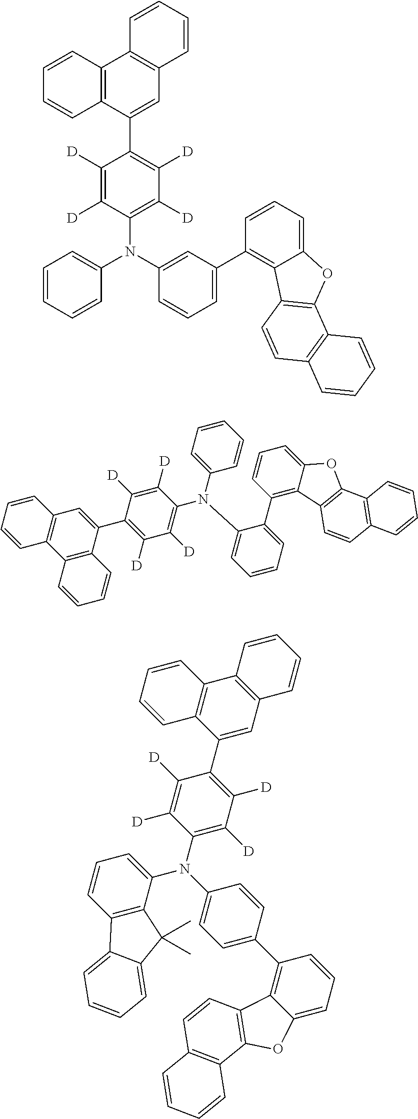 [SMILES: [2H]c1c([2H])c(N(c2ccccc2)c2cccc(-c3cccc4oc5c6ccccc6ccc5c34)c2)c([2H])c([2H])c1-c1cc2ccccc2c2ccccc12 ; [2H]c1c([2H])c(N(c2ccccc2)c2ccccc2-c2cccc3oc4c5ccccc5ccc4c23)c([2H])c([2H])c1-c1cc2ccccc2c2ccccc12 ; [2H]c1c([2H])c(N(c2ccc(-c3cccc4oc5c6ccccc6ccc5c34)cc2)c2cccc3c2C(C)(C)c2ccccc2-3)c([2H])c([2H])c1-c1cc2ccccc2c2ccccc12]

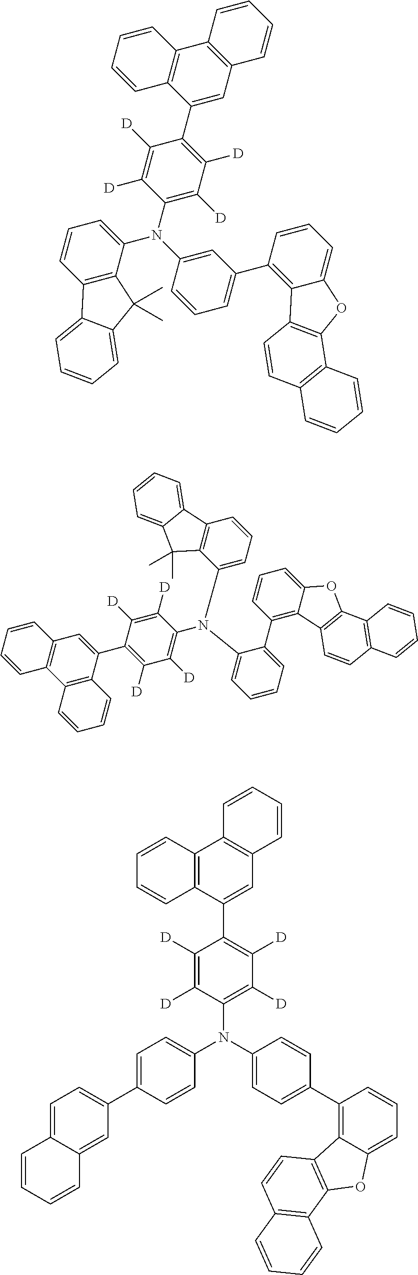 [SMILES: [2H]c1c([2H])c(N(c2cccc(-c3cccc4oc5c6ccccc6ccc5c34)c2)c2cccc3c2C(C)(C)c2ccccc2-3)c([2H])c([2H])c1-c1cc2ccccc2c2ccccc12 ; [2H]c1c([2H])c(N(c2ccccc2-c2cccc3oc4c5ccccc5ccc4c23)c2cccc3c2C(C)(C)c2ccccc2-3)c([2H])c([2H])c1-c1cc2ccccc2c2ccccc12 ; [2H]c1c([2H])c(N(c2ccc(-c3ccc4ccccc4c3)cc2)c2ccc(-c3cccc4oc5c6ccccc6ccc5c34)cc2)c([2H])c([2H])c1-c1cc2ccccc2c2ccccc12]

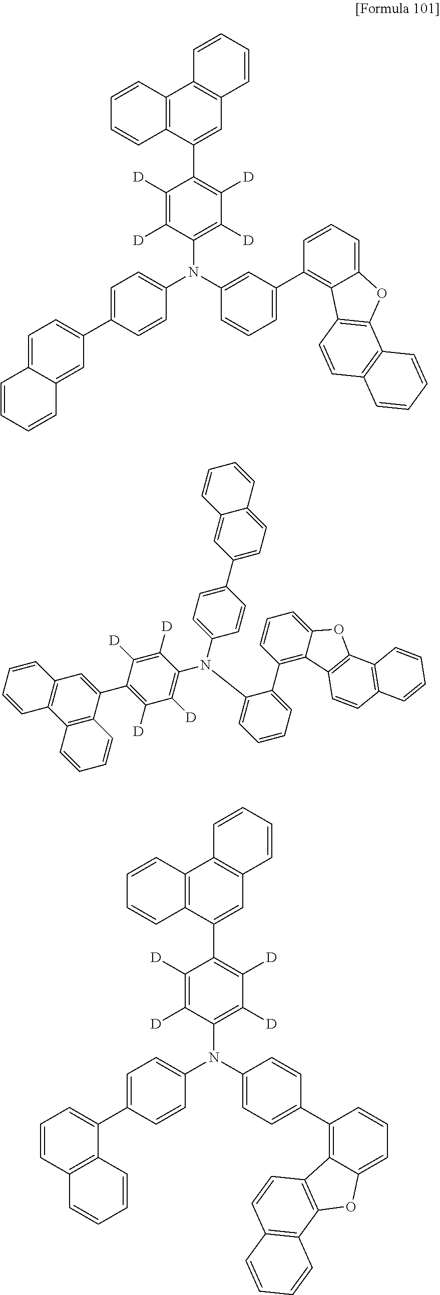 [SMILES: [2H]c1c([2H])c(N(c2ccc(-c3ccc4ccccc4c3)cc2)c2cccc(-c3cccc4oc5c6ccccc6ccc5c34)c2)c([2H])c([2H])c1-c1cc2ccccc2c2ccccc12 ; [2H]c1c([2H])c(N(c2ccc(-c3ccc4ccccc4c3)cc2)c2ccccc2-c2cccc3oc4c5ccccc5ccc4c23)c([2H])c([2H])c1-c1cc2ccccc2c2ccccc12 ; [2H]c1c([2H])c(N(c2ccc(-c3cccc4ccccc34)cc2)c2ccc(-c3cccc4oc5c6ccccc6ccc5c34)cc2)c([2H])c([2H])c1-c1cc2ccccc2c2ccccc12]

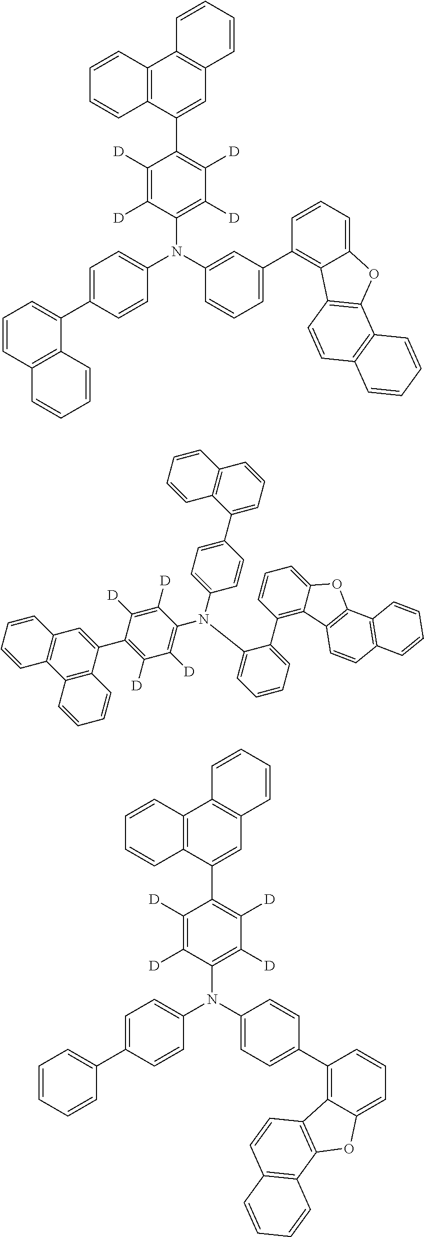 [SMILES: [2H]c1c([2H])c(N(c2ccc(-c3cccc4ccccc34)cc2)c2cccc(-c3cccc4oc5c6ccccc6ccc5c34)c2)c([2H])c([2H])c1-c1cc2ccccc2c2ccccc12 ; [2H]c1c([2H])c(N(c2ccc(-c3cccc4ccccc34)cc2)c2ccccc2-c2cccc3oc4c5ccccc5ccc4c23)c([2H])c([2H])c1-c1cc2ccccc2c2ccccc12 ; [2H]c1c([2H])c(N(c2ccc(-c3ccccc3)cc2)c2ccc(-c3cccc4oc5c6ccccc6ccc5c34)cc2)c([2H])c([2H])c1-c1cc2ccccc2c2ccccc12]

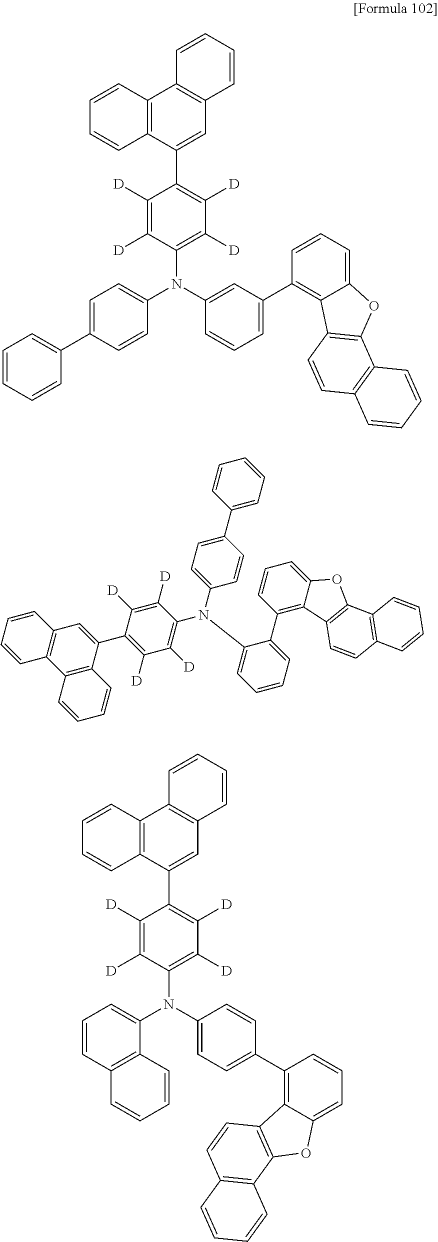 [SMILES: [2H]c1c([2H])c(N(c2ccc(-c3ccccc3)cc2)c2cccc(-c3cccc4oc5c6ccccc6ccc5c34)c2)c([2H])c([2H])c1-c1cc2ccccc2c2ccccc12 ; [2H]c1c([2H])c(N(c2ccc(-c3ccccc3)cc2)c2ccccc2-c2cccc3oc4c5ccccc5ccc4c23)c([2H])c([2H])c1-c1cc2ccccc2c2ccccc12 ; [2H]c1c([2H])c(N(c2ccc(-c3cccc4oc5c6ccccc6ccc5c34)cc2)c2cccc3ccccc23)c([2H])c([2H])c1-c1cc2ccccc2c2ccccc12]

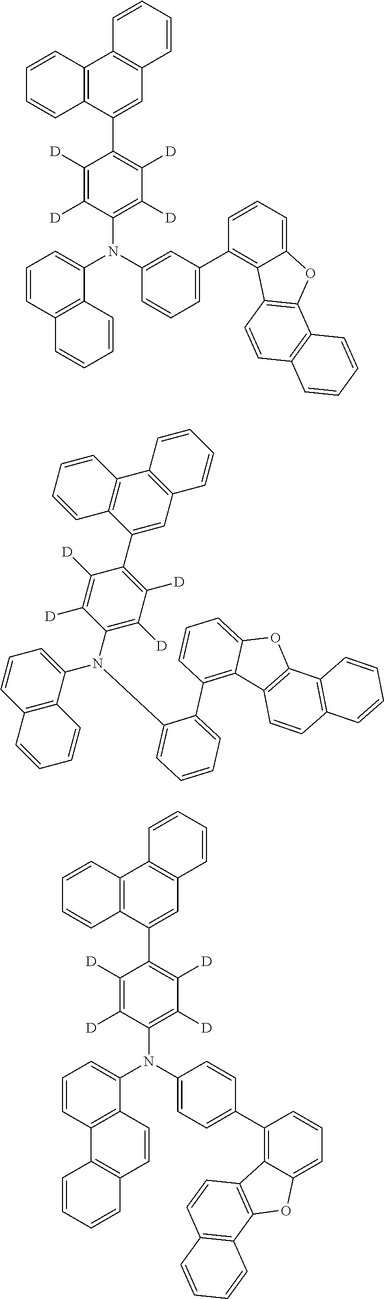

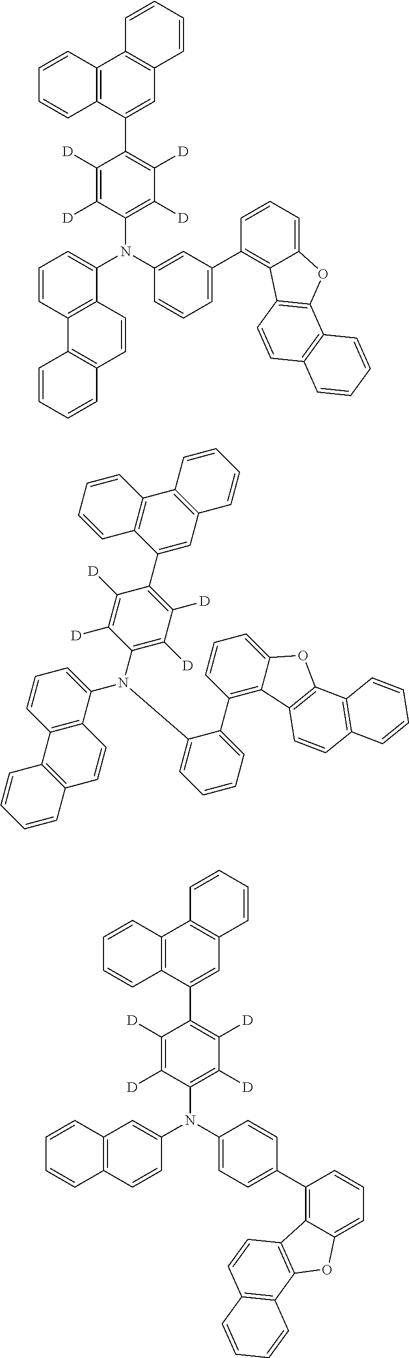 [SMILES: [2H]c1c([2H])c(N(c2cccc(-c3cccc4oc5c6ccccc6ccc5c34)c2)c2cccc3c2ccc2ccccc23)c([2H])c([2H])c1-c1cc2ccccc2c2ccccc12 ; [2H]c1c([2H])c(N(c2ccccc2-c2cccc3oc4c5ccccc5ccc4c23)c2cccc3c2ccc2ccccc23)c([2H])c([2H])c1-c1cc2ccccc2c2ccccc12 ; [2H]c1c([2H])c(N(c2ccc(-c3cccc4oc5c6ccccc6ccc5c34)cc2)c2ccc3ccccc3c2)c([2H])c([2H])c1-c1cc2ccccc2c2ccccc12]

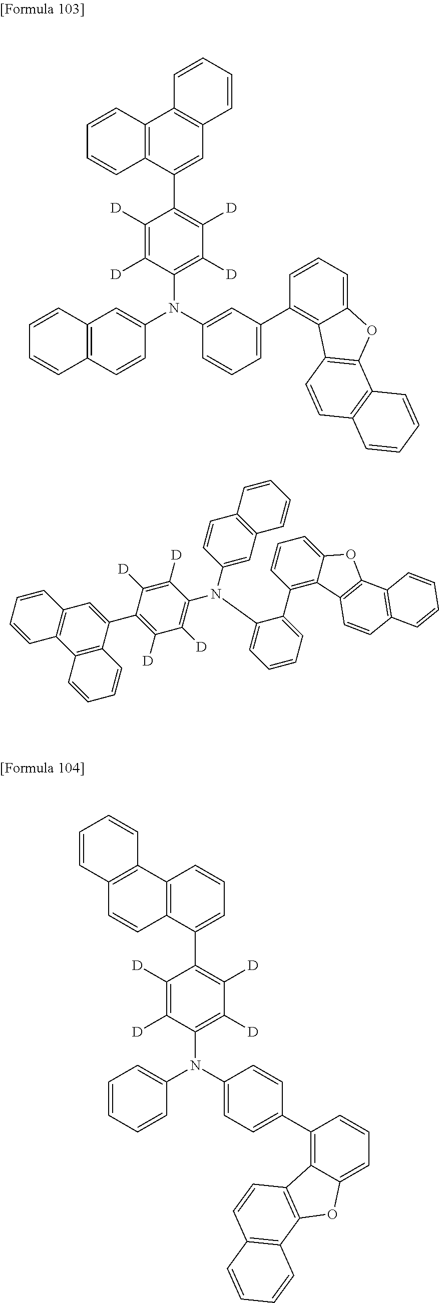 [SMILES: [2H]c1c([2H])c(N(c2cccc(-c3cccc4oc5c6ccccc6ccc5c34)c2)c2ccc3ccccc3c2)c([2H])c([2H])c1-c1cc2ccccc2c2ccccc12 ; [2H]c1c([2H])c(N(c2ccc3ccccc3c2)c2ccccc2-c2cccc3oc4c5ccccc5ccc4c23)c([2H])c([2H])c1-c1cc2ccccc2c2ccccc12 ; [2H]c1c([2H])c(N(c2ccccc2)c2ccc(-c3cccc4oc5c6ccccc6ccc5c34)cc2)c([2H])c([2H])c1-c1cccc2c1ccc1ccccc12]

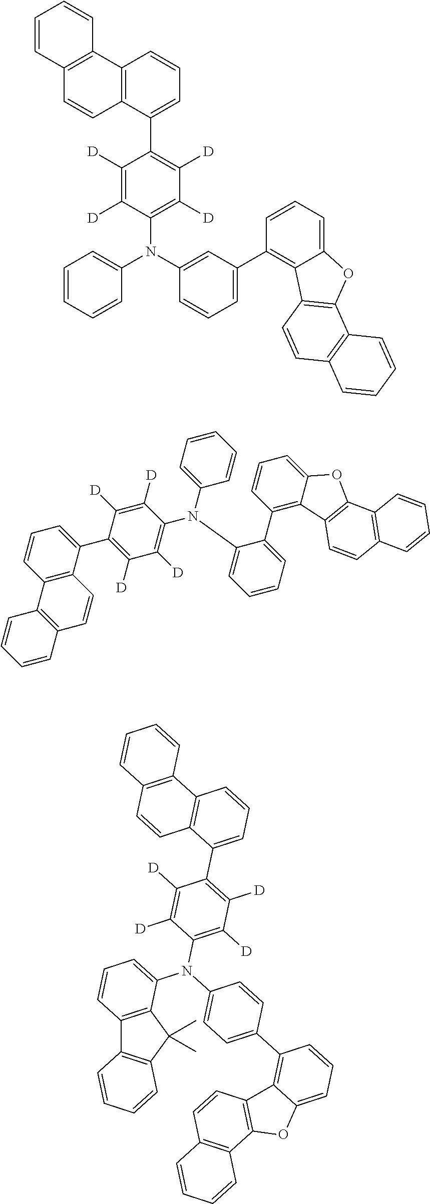 [SMILES: [2H]c1c([2H])c(N(c2ccccc2)c2cccc(-c3cccc4oc5c6ccccc6ccc5c34)c2)c([2H])c([2H])c1-c1cccc2c1ccc1ccccc12 ; [2H]c1c([2H])c(N(c2ccccc2)c2ccccc2-c2cccc3oc4c5ccccc5ccc4c23)c([2H])c([2H])c1-c1cccc2c1ccc1ccccc12 ; [2H]c1c([2H])c(N(c2ccc(-c3cccc4oc5c6ccccc6ccc5c34)cc2)c2cccc3c2C(C)(C)c2ccccc2-3)c([2H])c([2H])c1-c1cccc2c1ccc1ccccc12]

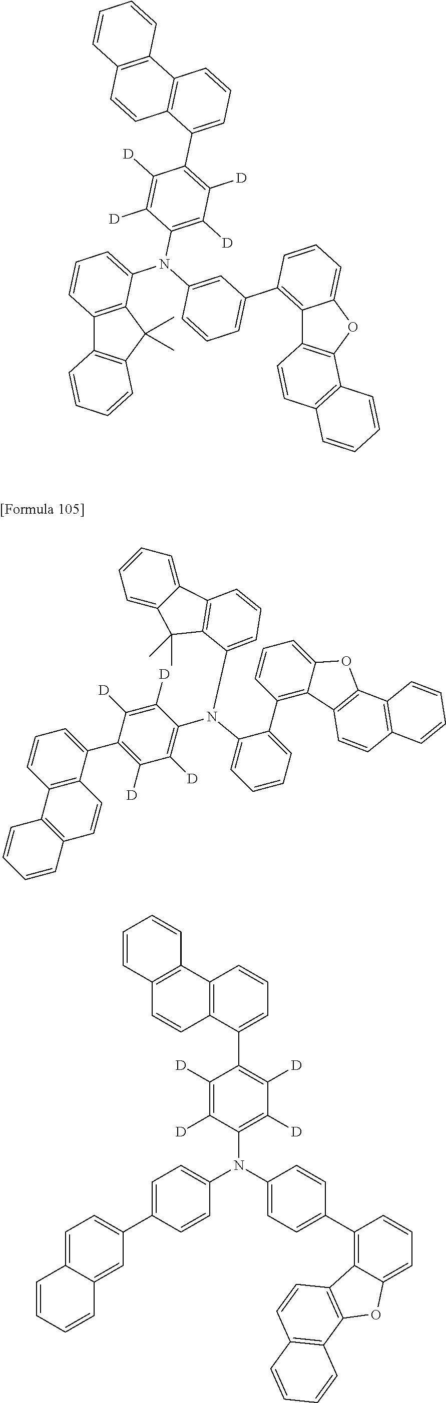 [SMILES: [2H]c1c([2H])c(N(c2cccc(-c3cccc4oc5c6ccccc6ccc5c34)c2)c2cccc3c2C(C)(C)c2ccccc2-3)c([2H])c([2H])c1-c1cccc2c1ccc1ccccc12 ; [2H]c1c([2H])c(N(c2ccccc2-c2cccc3oc4c5ccccc5ccc4c23)c2cccc3c2C(C)(C)c2ccccc2-3)c([2H])c([2H])c1-c1cccc2c1ccc1ccccc12 ; [2H]c1c([2H])c(N(c2ccc(-c3ccc4ccccc4c3)cc2)c2ccc(-c3cccc4oc5c6ccccc6ccc5c34)cc2)c([2H])c([2H])c1-c1cccc2c1ccc1ccccc12]

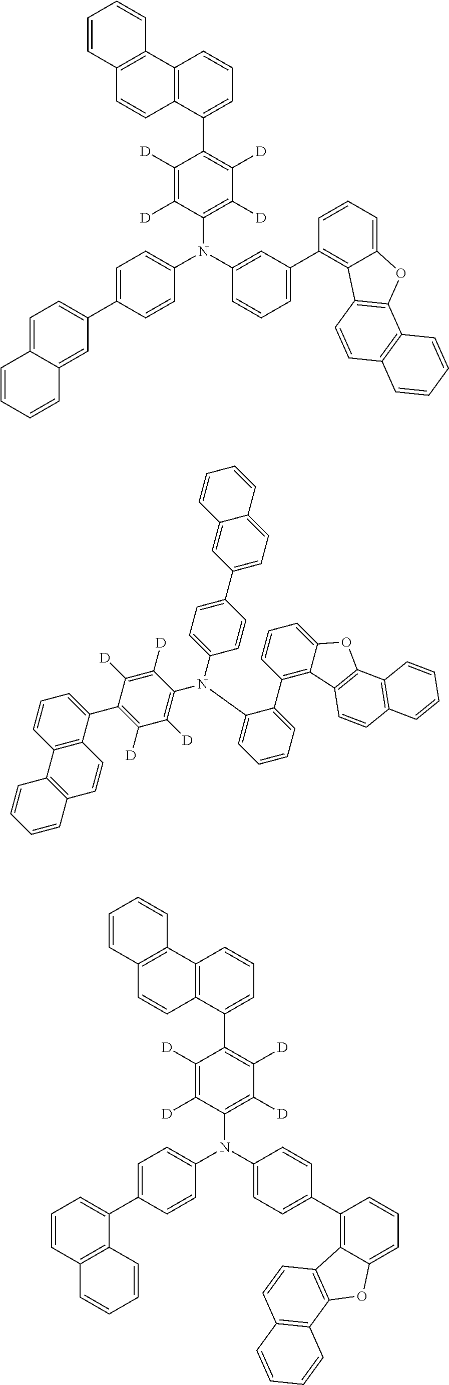 [SMILES: [2H]c1c([2H])c(N(c2ccc(-c3ccc4ccccc4c3)cc2)c2cccc(-c3cccc4oc5c6ccccc6ccc5c34)c2)c([2H])c([2H])c1-c1cccc2c1ccc1ccccc12 ; [2H]c1c([2H])c(N(c2ccc(-c3ccc4ccccc4c3)cc2)c2ccccc2-c2cccc3oc4c5ccccc5ccc4c23)c([2H])c([2H])c1-c1cccc2c1ccc1ccccc12 ; [2H]c1c([2H])c(N(c2ccc(-c3cccc4ccccc34)cc2)c2ccc(-c3cccc4oc5c6ccccc6ccc5c34)cc2)c([2H])c([2H])c1-c1cccc2c1ccc1ccccc12]

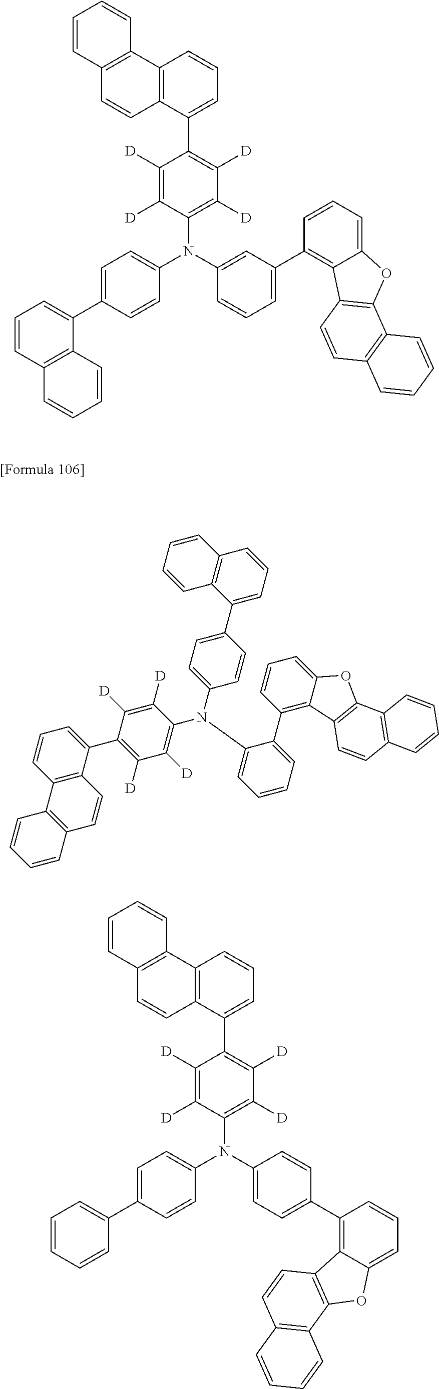 [SMILES: [2H]c1c([2H])c(N(c2ccc(-c3cccc4ccccc34)cc2)c2cccc(-c3cccc4oc5c6ccccc6ccc5c34)c2)c([2H])c([2H])c1-c1cccc2c1ccc1ccccc12 ; [2H]c1c([2H])c(N(c2ccc(-c3cccc4ccccc34)cc2)c2ccccc2-c2cccc3oc4c5ccccc5ccc4c23)c([2H])c([2H])c1-c1cccc2c1ccc1ccccc12 ; [2H]c1c([2H])c(N(c2ccc(-c3ccccc3)cc2)c2ccc(-c3cccc4oc5c6ccccc6ccc5c34)cc2)c([2H])c([2H])c1-c1cccc2c1ccc1ccccc12]

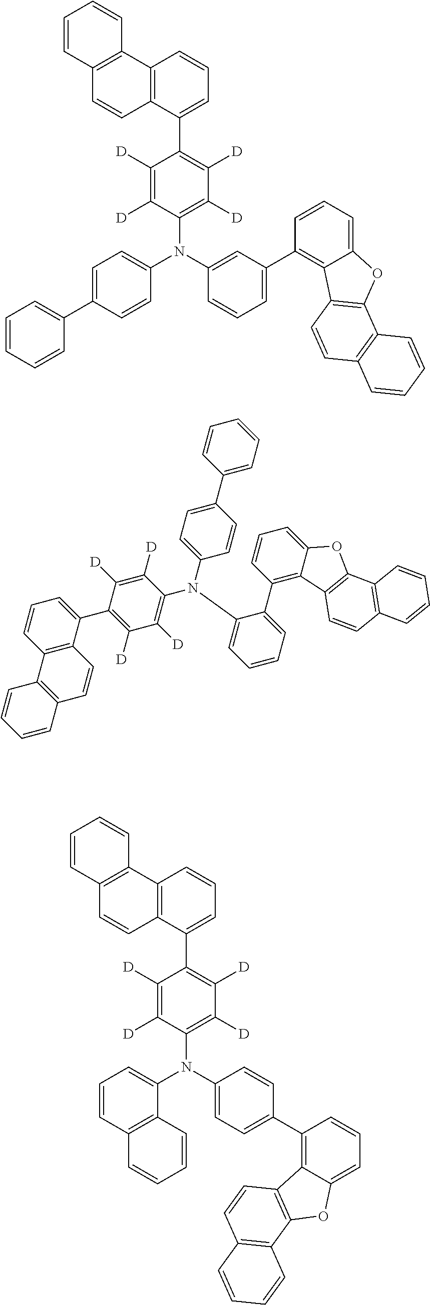 [SMILES: [2H]c1c([2H])c(N(c2ccc(-c3ccccc3)cc2)c2cccc(-c3cccc4oc5c6ccccc6ccc5c34)c2)c([2H])c([2H])c1-c1cccc2c1ccc1ccccc12 ; [2H]c1c([2H])c(N(c2ccc(-c3ccccc3)cc2)c2ccccc2-c2cccc3oc4c5ccccc5ccc4c23)c([2H])c([2H])c1-c1cccc2c1ccc1ccccc12 ; [2H]c1c([2H])c(N(c2ccc(-c3cccc4oc5c6ccccc6ccc5c34)cc2)c2cccc3ccccc23)c([2H])c([2H])c1-c1cccc2c1ccc1ccccc12]

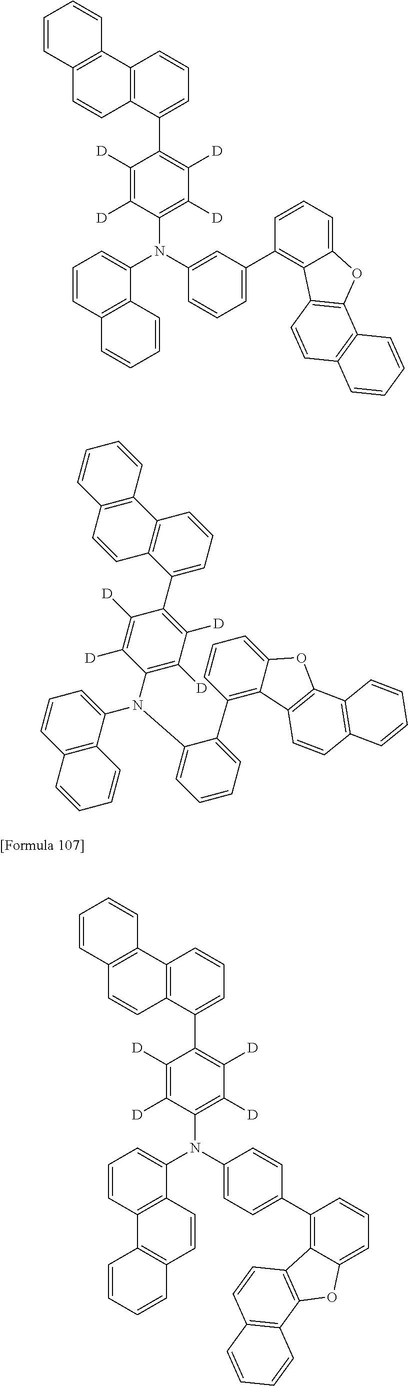 [SMILES: [2H]c1c([2H])c(N(c2cccc(-c3cccc4oc5c6ccccc6ccc5c34)c2)c2cccc3ccccc23)c([2H])c([2H])c1-c1cccc2c1ccc1ccccc12 ; [2H]c1c([2H])c(N(c2ccccc2-c2cccc3oc4c5ccccc5ccc4c23)c2cccc3ccccc23)c([2H])c([2H])c1-c1cccc2c1ccc1ccccc12 ; [2H]c1c([2H])c(N(c2ccc(-c3cccc4oc5c6ccccc6ccc5c34)cc2)c2cccc3c2ccc2ccccc23)c([2H])c([2H])c1-c1cccc2c1ccc1ccccc12]

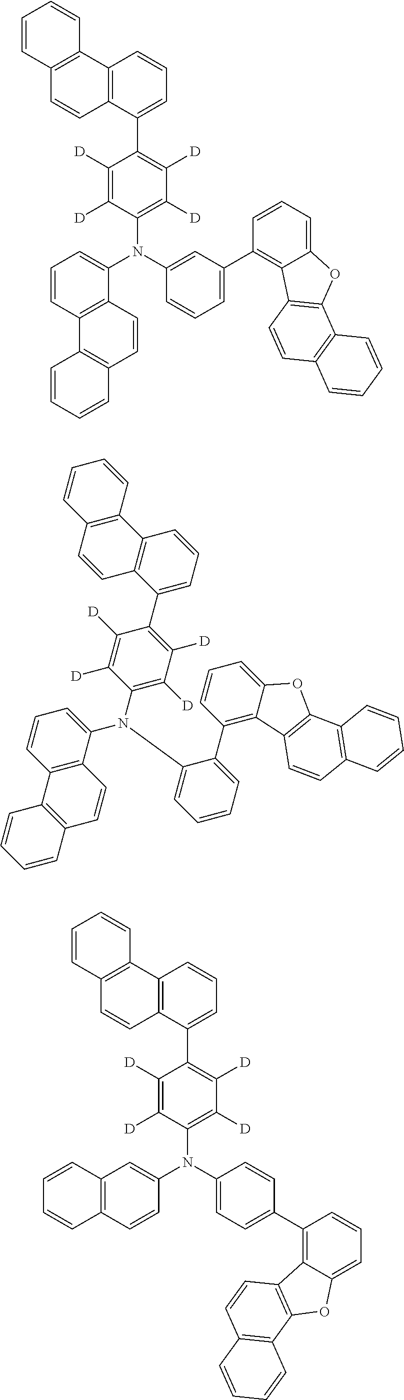 [SMILES: [2H]c1c([2H])c(N(c2cccc(-c3cccc4oc5c6ccccc6ccc5c34)c2)c2cccc3c2ccc2ccccc23)c([2H])c([2H])c1-c1cccc2c1ccc1ccccc12 ; [2H]c1c([2H])c(N(c2ccccc2-c2cccc3oc4c5ccccc5ccc4c23)c2cccc3c2ccc2ccccc23)c([2H])c([2H])c1-c1cccc2c1ccc1ccccc12 ; [2H]c1c([2H])c(N(c2ccc(-c3cccc4oc5c6ccccc6ccc5c34)cc2)c2ccc3ccccc3c2)c([2H])c([2H])c1-c1cccc2c1ccc1ccccc12]

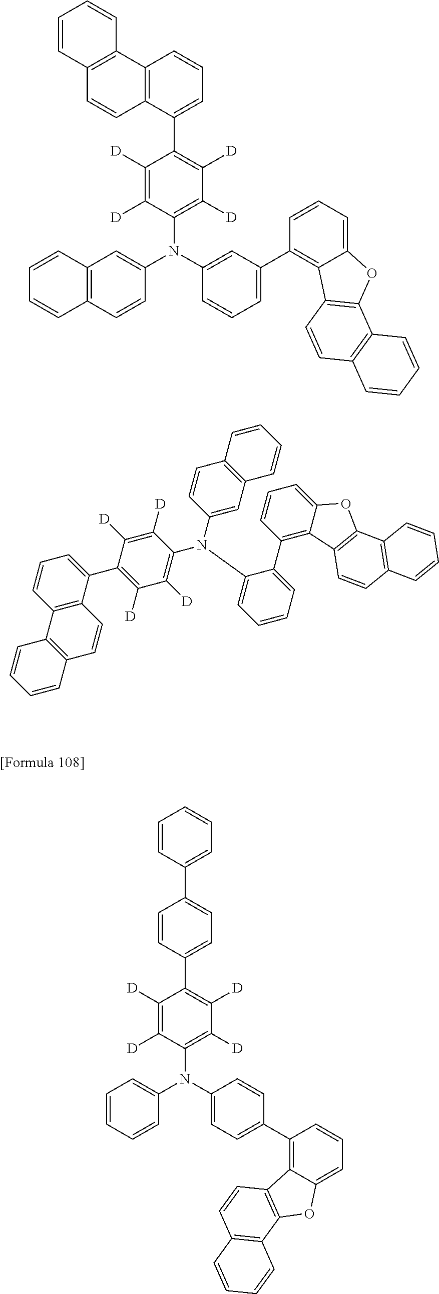 [SMILES: [2H]c1c([2H])c(N(c2cccc(-c3cccc4oc5c6ccccc6ccc5c34)c2)c2ccc3ccccc3c2)c([2H])c([2H])c1-c1cccc2c1ccc1ccccc12 ; [2H]c1c([2H])c(N(c2ccc3ccccc3c2)c2ccccc2-c2cccc3oc4c5ccccc5ccc4c23)c([2H])c([2H])c1-c1cccc2c1ccc1ccccc12 ; [2H]c1c([2H])c(N(c2ccccc2)c2ccc(-c3cccc4oc5c6ccccc6ccc5c34)cc2)c([2H])c([2H])c1-c1ccc(-c2ccccc2)cc1]

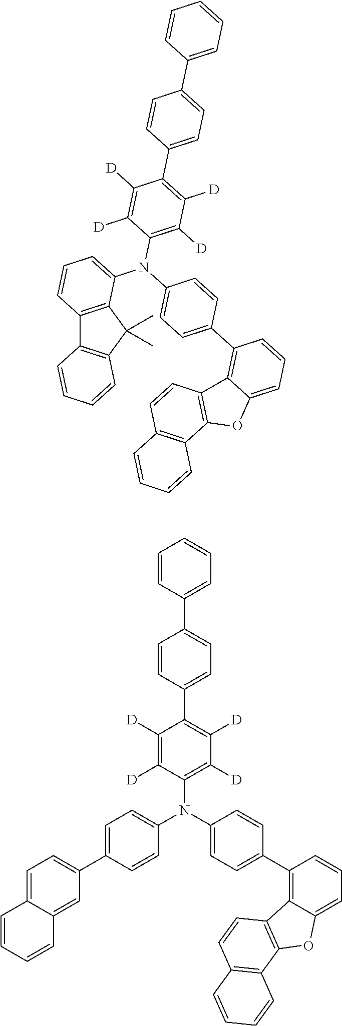 [SMILES: [2H]c1c([2H])c(N(c2ccc(-c3cccc4oc5c6ccccc6ccc5c34)cc2)c2cccc3c2C(C)(C)c2ccccc2-3)c([2H])c([2H])c1-c1ccc(-c2ccccc2)cc1 ; [2H]c1c([2H])c(N(c2ccc(-c3ccc4ccccc4c3)cc2)c2ccc(-c3cccc4oc5c6ccccc6ccc5c34)cc2)c([2H])c([2H])c1-c1ccc(-c2ccccc2)cc1]

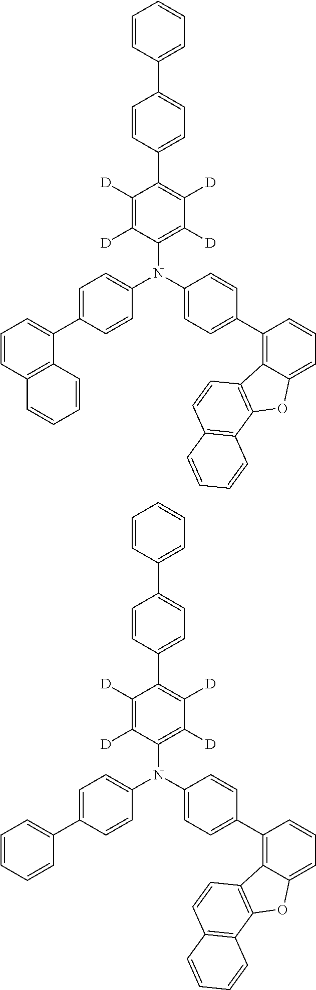

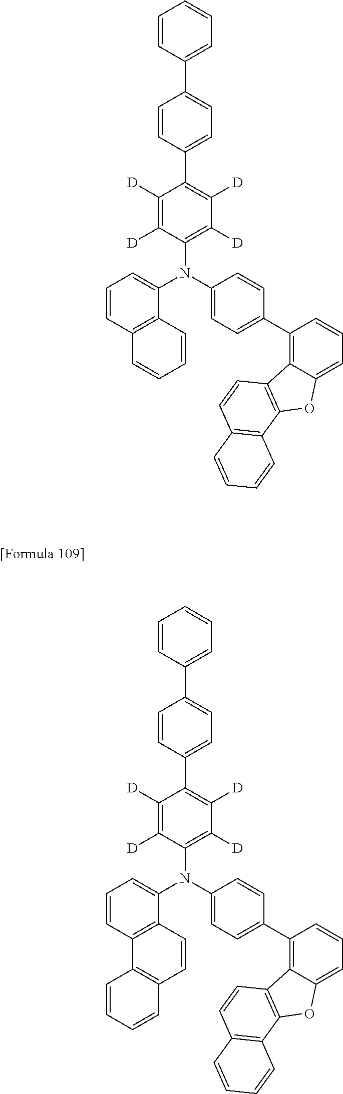 [SMILES: [2H]c1c([2H])c(N(c2ccc(-c3cccc4oc5c6ccccc6ccc5c34)cc2)c2cccc3ccccc23)c([2H])c([2H])c1-c1ccc(-c2ccccc2)cc1 ; [2H]c1c([2H])c(N(c2ccc(-c3cccc4oc5c6ccccc6ccc5c34)cc2)c2cccc3c2ccc2ccccc23)c([2H])c([2H])c1-c1ccc(-c2ccccc2)cc1]

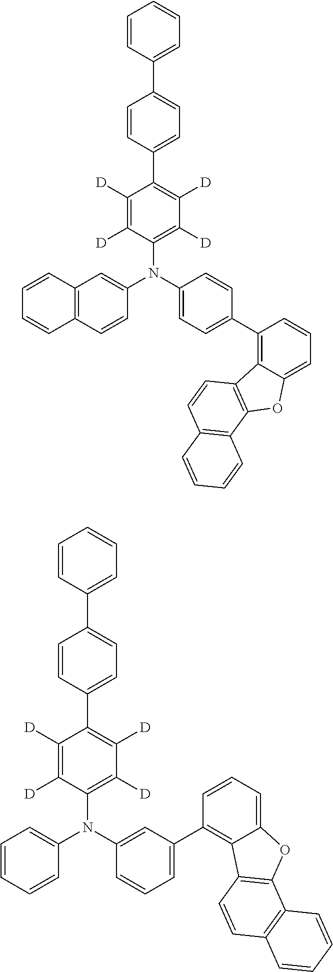 [SMILES: [2H]c1c([2H])c(N(c2ccc(-c3cccc4oc5c6ccccc6ccc5c34)cc2)c2ccc3ccccc3c2)c([2H])c([2H])c1-c1ccc(-c2ccccc2)cc1 ; [2H]c1c([2H])c(N(c2ccccc2)c2cccc(-c3cccc4oc5c6ccccc6ccc5c34)c2)c([2H])c([2H])c1-c1ccc(-c2ccccc2)cc1]

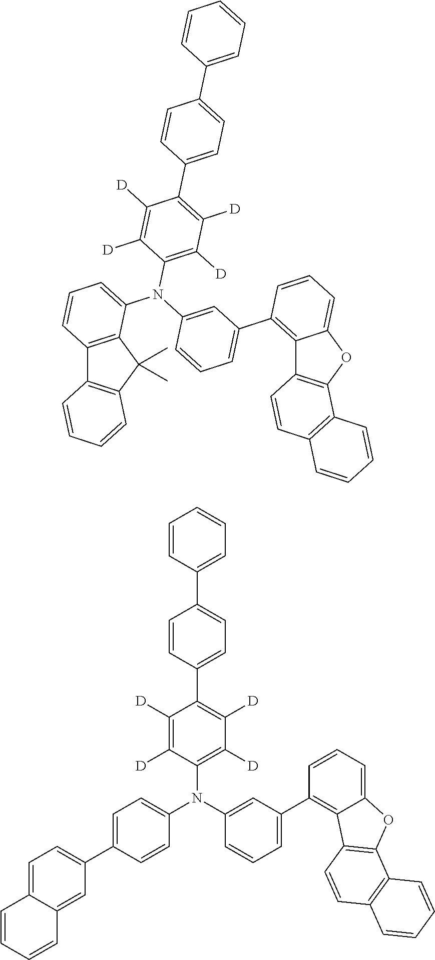 [SMILES: [2H]c1c([2H])c(N(c2cccc(-c3cccc4oc5c6ccccc6ccc5c34)c2)c2cccc3c2C(C)(C)c2ccccc2-3)c([2H])c([2H])c1-c1ccc(-c2ccccc2)cc1 ; [2H]c1c([2H])c(N(c2ccc(-c3ccc4ccccc4c3)cc2)c2cccc(-c3cccc4oc5c6ccccc6ccc5c34)c2)c([2H])c([2H])c1-c1ccc(-c2ccccc2)cc1]

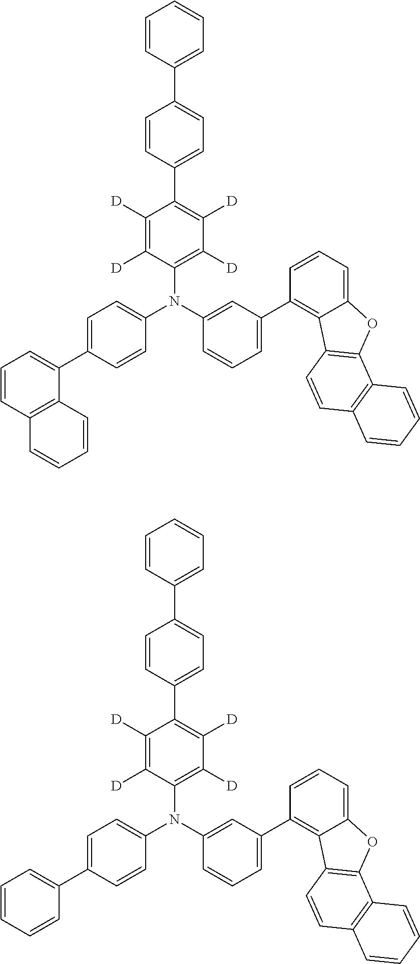 [SMILES: [2H]c1c([2H])c(N(c2ccc(-c3cccc4ccccc34)cc2)c2cccc(-c3cccc4oc5c6ccccc6ccc5c34)c2)c([2H])c([2H])c1-c1ccc(-c2ccccc2)cc1 ; [2H]c1c([2H])c(N(c2ccc(-c3ccccc3)cc2)c2cccc(-c3cccc4oc5c6ccccc6ccc5c34)c2)c([2H])c([2H])c1-c1ccc(-c2ccccc2)cc1]

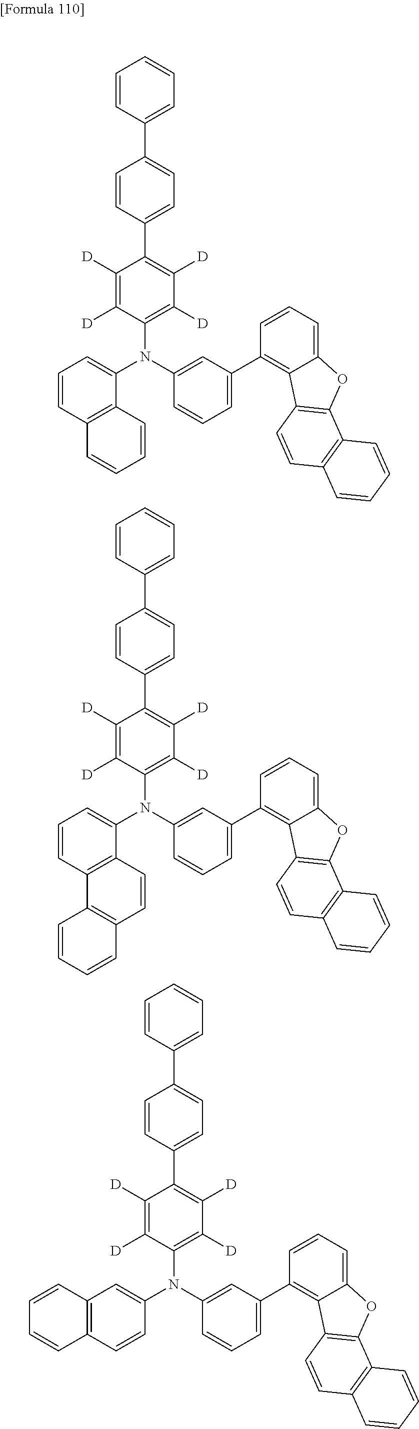

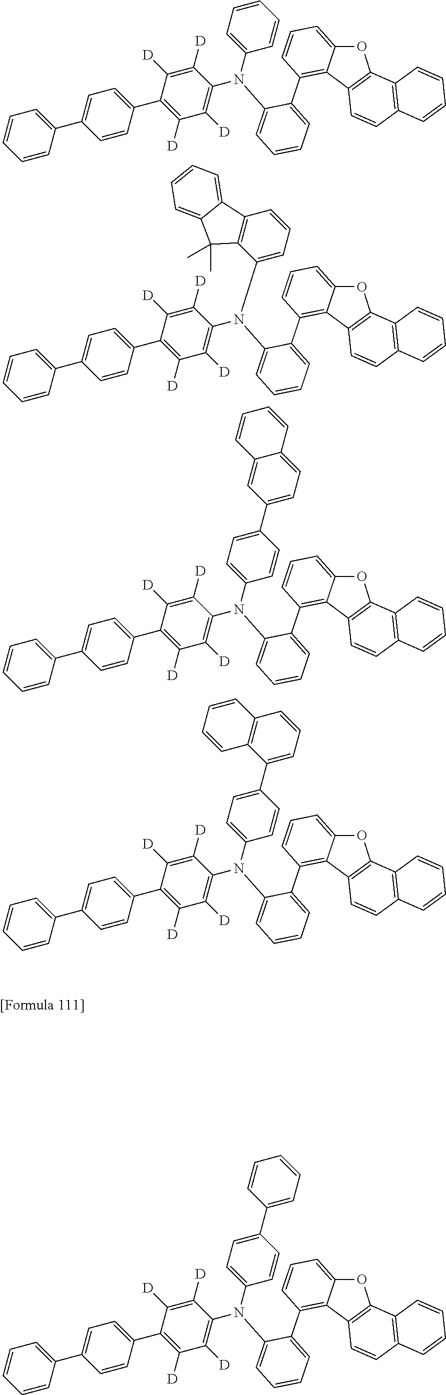

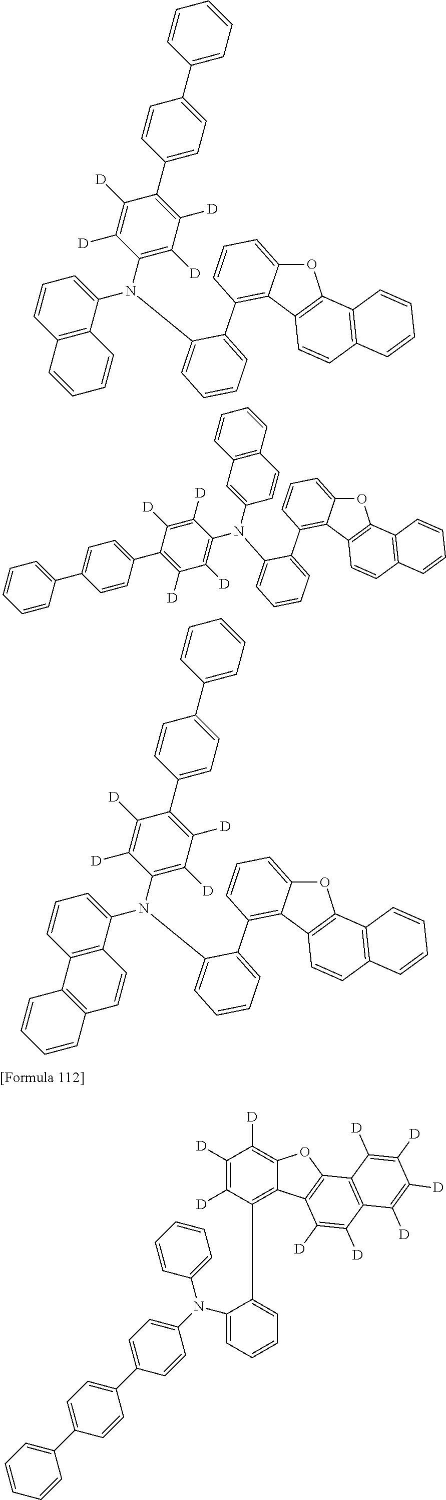

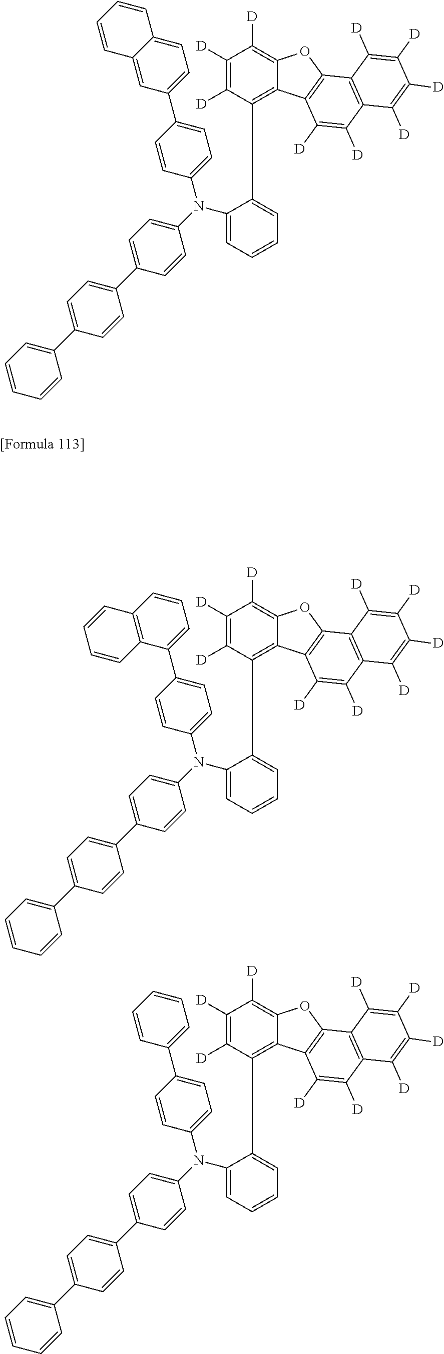 [SMILES: [2H]c1c([2H])c(-c2ccccc2N(c2ccc(-c3ccc(-c4ccccc4)cc3)cc2)c2ccc(-c3ccc4ccccc4c3)cc2)c2c(oc3c4c([2H])c([2H])c([2H])c([2H])c4c([2H])c([2H])c32)c1[2H] ; [2H]c1c([2H])c(-c2ccccc2N(c2ccc(-c3ccc(-c4ccccc4)cc3)cc2)c2ccc(-c3cccc4ccccc34)cc2)c2c(oc3c4c([2H])c([2H])c([2H])c([2H])c4c([2H])c([2H])c32)c1[2H] ; [2H]c1c([2H])c(-c2ccccc2N(c2ccc(-c3ccccc3)cc2)c2ccc(-c3ccc(-c4ccccc4)cc3)cc2)c2c(oc3c4c([2H])c([2H])c([2H])c([2H])c4c([2H])c([2H])c32)c1[2H]]

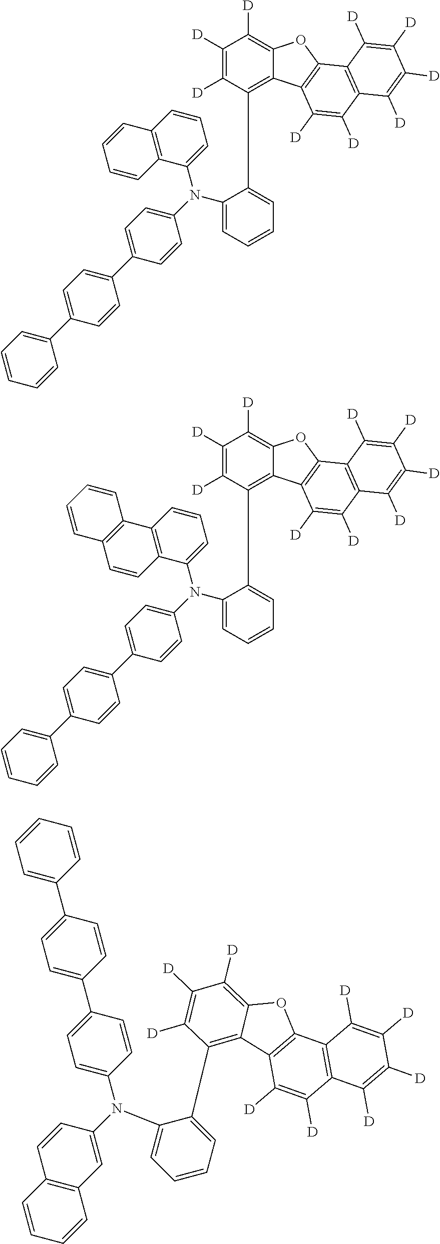

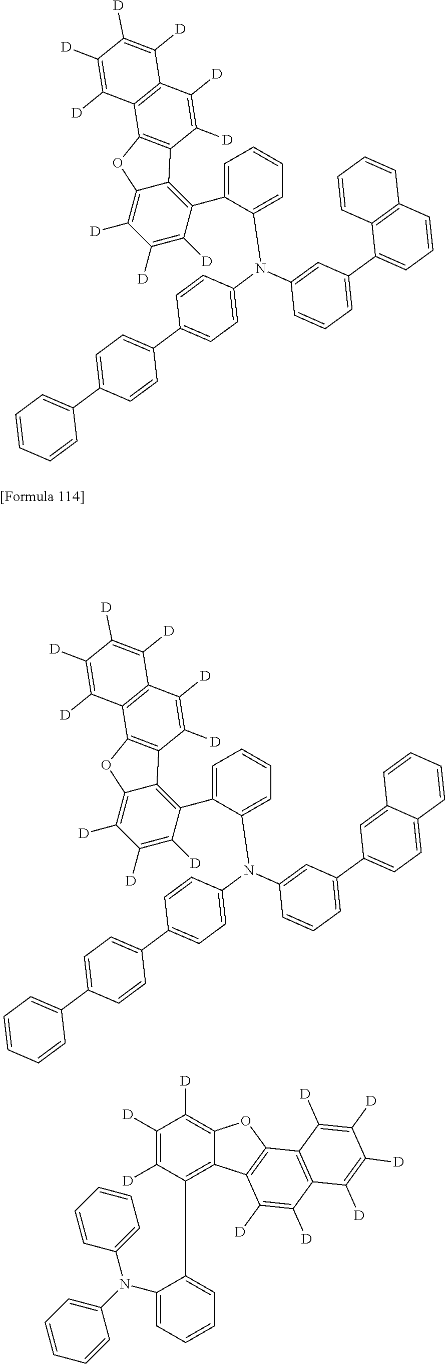 [SMILES: [2H]c1c([2H])c(-c2ccccc2N(c2ccc(-c3ccc(-c4ccccc4)cc3)cc2)c2cccc(-c3cccc4ccccc34)c2)c2c(oc3c4c([2H])c([2H])c([2H])c([2H])c4c([2H])c([2H])c32)c1[2H] ; [2H]c1c([2H])c(-c2ccccc2N(c2ccc(-c3ccc(-c4ccccc4)cc3)cc2)c2cccc(-c3ccc4ccccc4c3)c2)c2c(oc3c4c([2H])c([2H])c([2H])c([2H])c4c([2H])c([2H])c32)c1[2H] ; [2H]c1c([2H])c(-c2ccccc2N(c2ccccc2)c2ccccc2)c2c(oc3c4c([2H])c([2H])c([2H])c([2H])c4c([2H])c([2H])c32)c1[2H]]

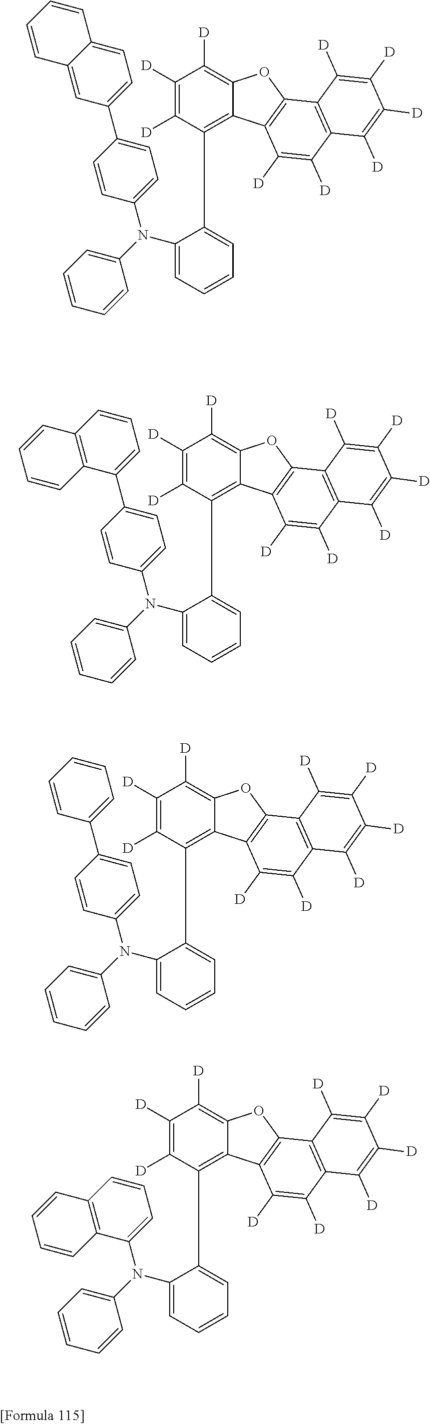 [SMILES: [2H]c1c([2H])c(-c2ccccc2N(c2ccccc2)c2ccc(-c3ccc4ccccc4c3)cc2)c2c(oc3c4c([2H])c([2H])c([2H])c([2H])c4c([2H])c([2H])c32)c1[2H] ; [2H]c1c([2H])c(-c2ccccc2N(c2ccccc2)c2ccc(-c3cccc4ccccc34)cc2)c2c(oc3c4c([2H])c([2H])c([2H])c([2H])c4c([2H])c([2H])c32)c1[2H] ; [2H]c1c([2H])c(-c2ccccc2N(c2ccccc2)c2ccc(-c3ccccc3)cc2)c2c(oc3c4c([2H])c([2H])c([2H])c([2H])c4c([2H])c([2H])c32)c1[2H] ; [2H]c1c([2H])c(-c2ccccc2N(c2ccccc2)c2cccc3ccccc23)c2c(oc3c4c([2H])c([2H])c([2H])c([2H])c4c([2H])c([2H])c32)c1[2H]]

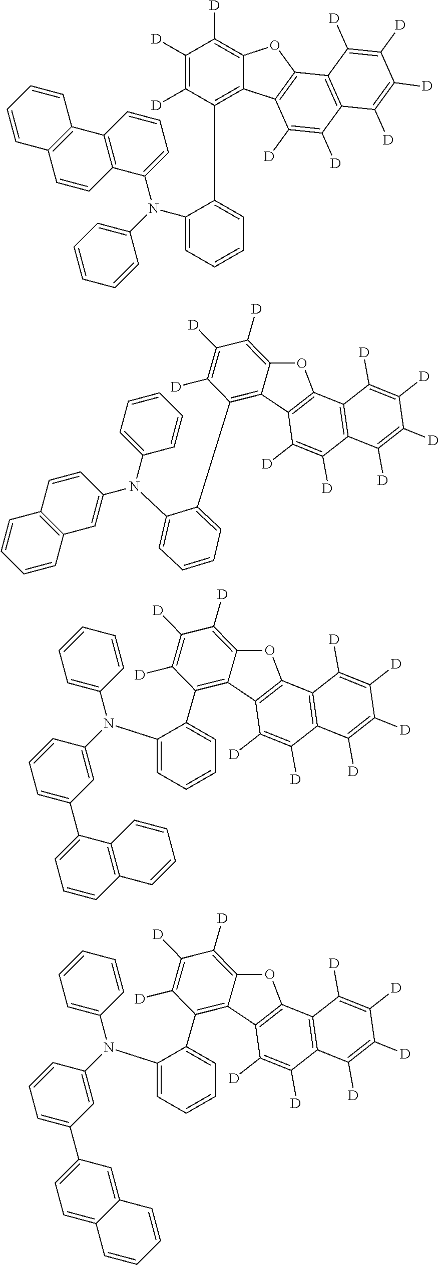

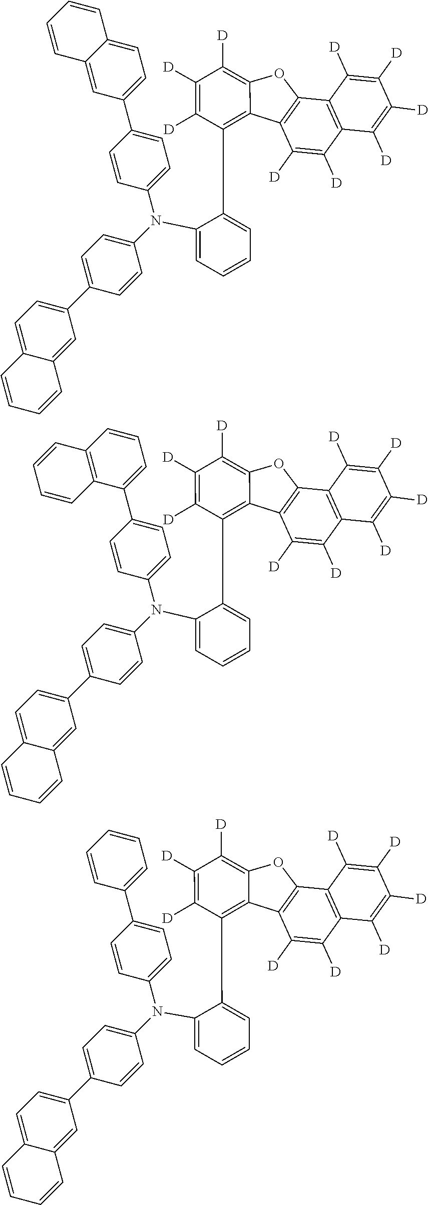

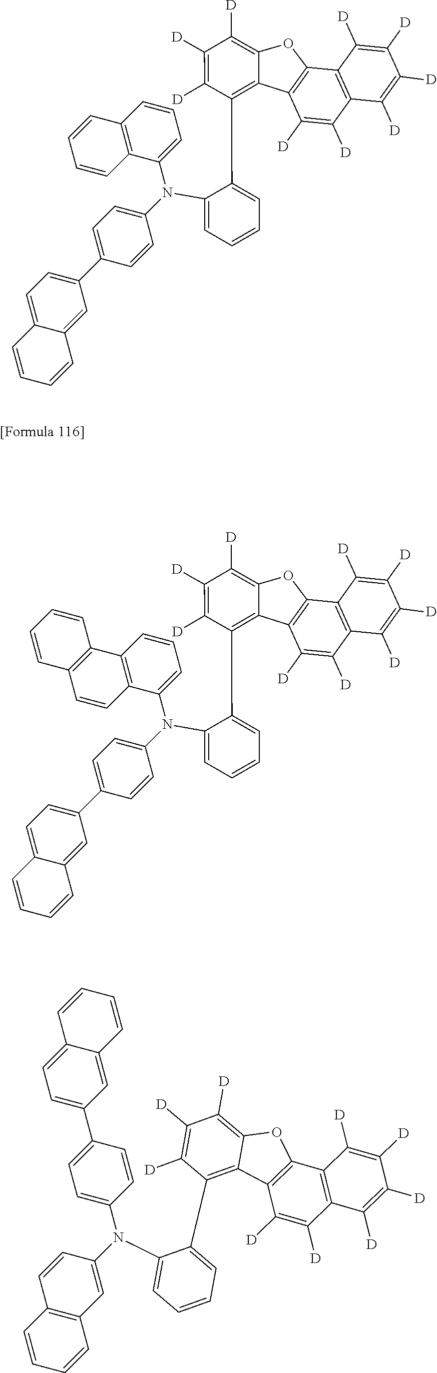 [SMILES: [2H]c1c([2H])c(-c2ccccc2N(c2ccc(-c3ccc4ccccc4c3)cc2)c2cccc3ccccc23)c2c(oc3c4c([2H])c([2H])c([2H])c([2H])c4c([2H])c([2H])c32)c1[2H] ; [2H]c1c([2H])c(-c2ccccc2N(c2ccc(-c3ccc4ccccc4c3)cc2)c2cccc3c2ccc2ccccc23)c2c(oc3c4c([2H])c([2H])c([2H])c([2H])c4c([2H])c([2H])c32)c1[2H] ; [2H]c1c([2H])c(-c2ccccc2N(c2ccc(-c3ccc4ccccc4c3)cc2)c2ccc3ccccc3c2)c2c(oc3c4c([2H])c([2H])c([2H])c([2H])c4c([2H])c([2H])c32)c1[2H]]

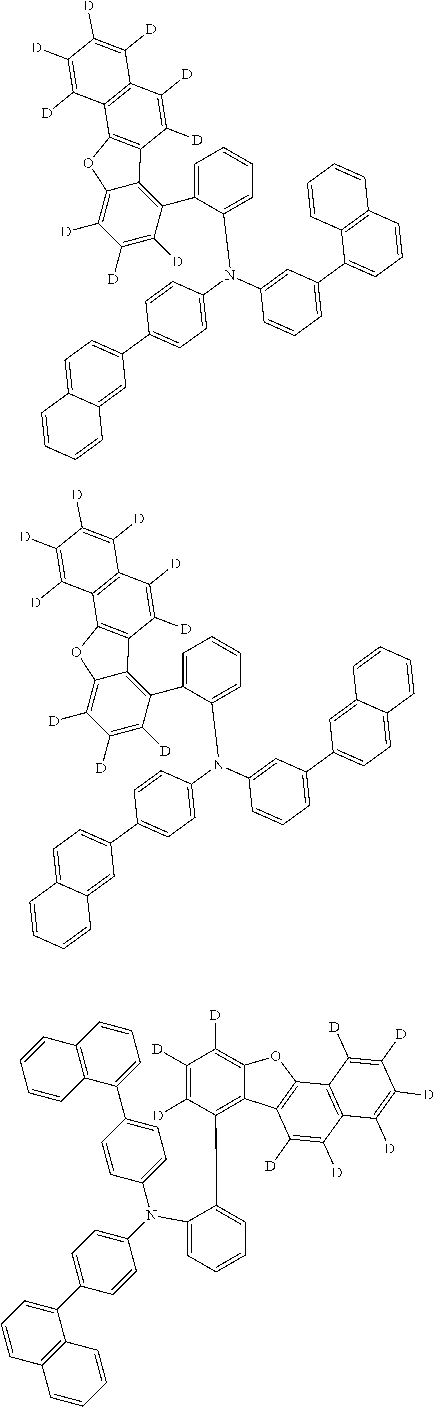

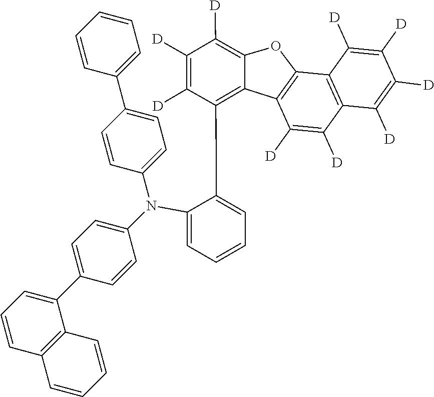 [SMILES: [2H]c1c([2H])c(-c2ccccc2N(c2ccc(-c3ccccc3)cc2)c2ccc(-c3cccc4ccccc34)cc2)c2c(oc3c4c([2H])c([2H])c([2H])c([2H])c4c([2H])c([2H])c32)c1[2H]]

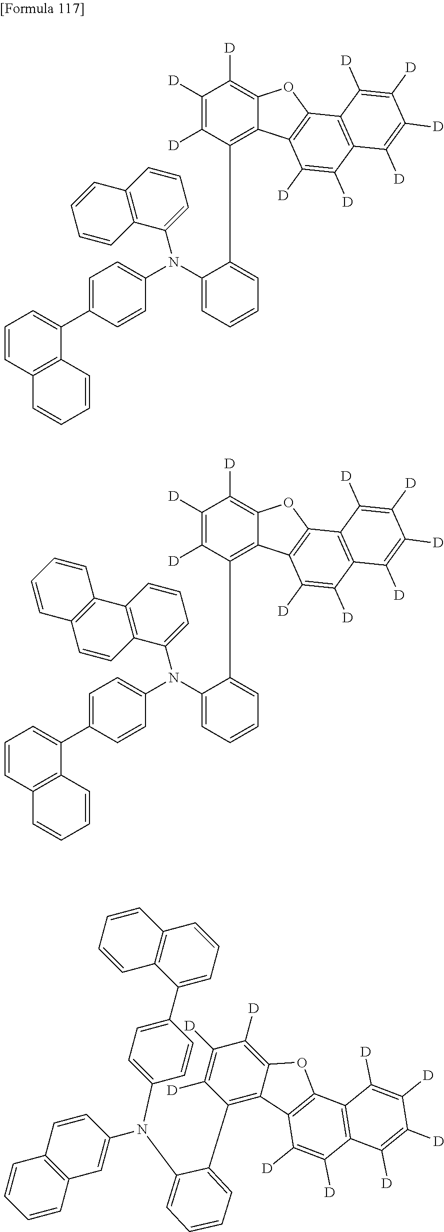 [SMILES: [2H]c1c([2H])c(-c2ccccc2N(c2ccc(-c3cccc4ccccc34)cc2)c2cccc3ccccc23)c2c(oc3c4c([2H])c([2H])c([2H])c([2H])c4c([2H])c([2H])c32)c1[2H] ; [2H]c1c([2H])c(-c2ccccc2N(c2ccc(-c3cccc4ccccc34)cc2)c2cccc3c2ccc2ccccc23)c2c(oc3c4c([2H])c([2H])c([2H])c([2H])c4c([2H])c([2H])c32)c1[2H] ; [2H]c1c([2H])c(-c2ccccc2N(c2ccc(-c3cccc4ccccc34)cc2)c2ccc3ccccc3c2)c2c(oc3c4c([2H])c([2H])c([2H])c([2H])c4c([2H])c([2H])c32)c1[2H]]

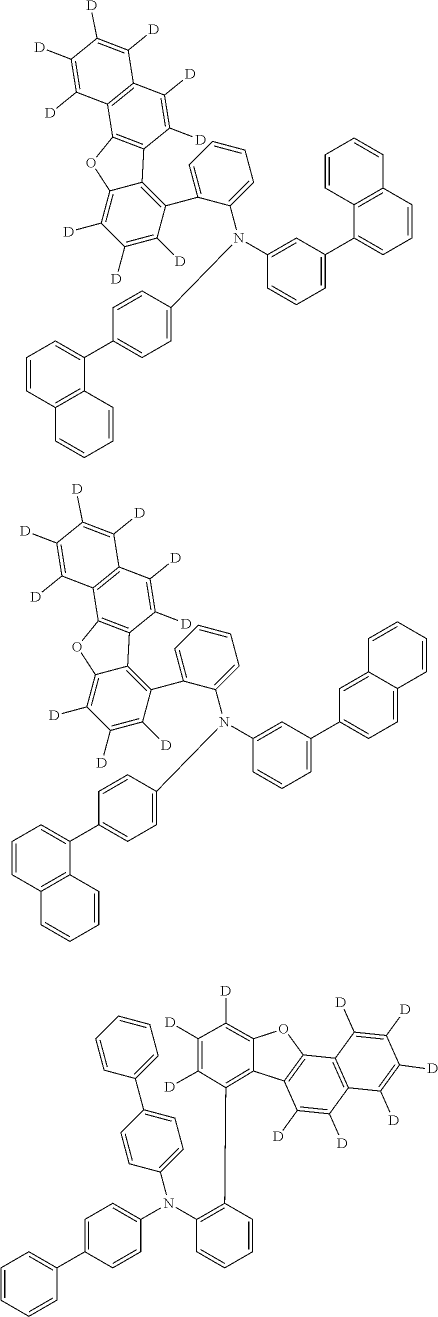

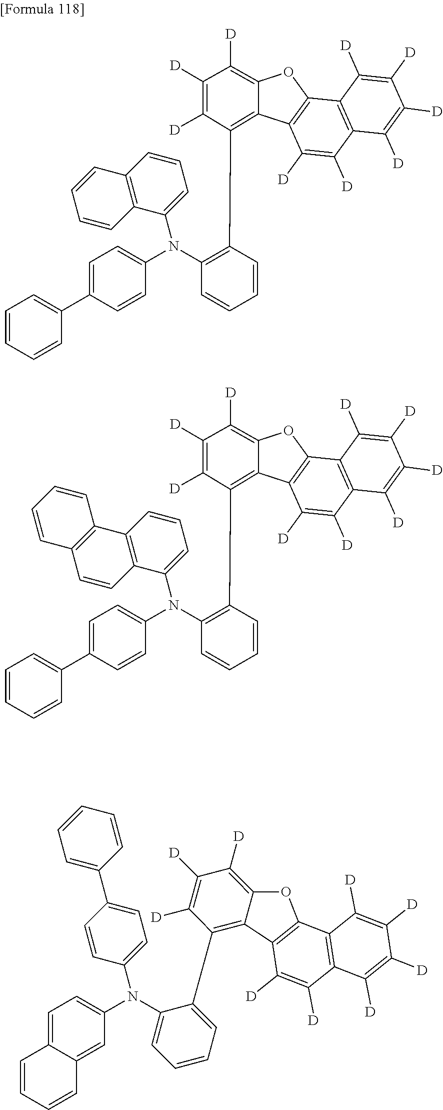 [SMILES: [2H]c1c([2H])c(-c2ccccc2N(c2ccc(-c3ccccc3)cc2)c2cccc3ccccc23)c2c(oc3c4c([2H])c([2H])c([2H])c([2H])c4c([2H])c([2H])c32)c1[2H] ; [2H]c1c([2H])c(-c2ccccc2N(c2ccc(-c3ccccc3)cc2)c2cccc3c2ccc2ccccc23)c2c(oc3c4c([2H])c([2H])c([2H])c([2H])c4c([2H])c([2H])c32)c1[2H] ; [2H]c1c([2H])c(-c2ccccc2N(c2ccc(-c3ccccc3)cc2)c2ccc3ccccc3c2)c2c(oc3c4c([2H])c([2H])c([2H])c([2H])c4c([2H])c([2H])c32)c1[2H]]

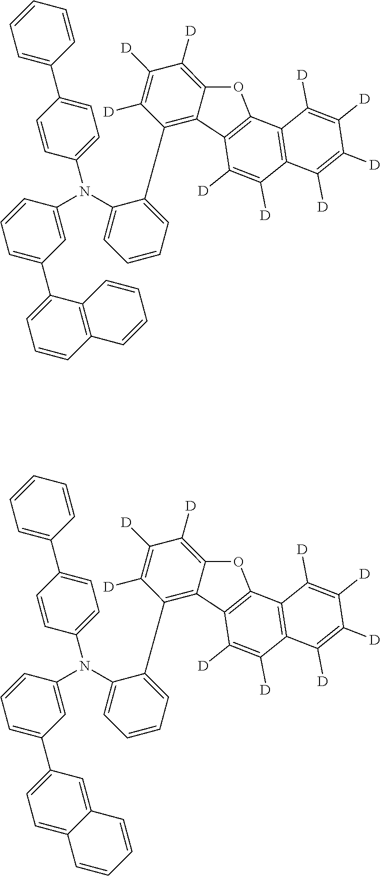 [SMILES: [2H]c1c([2H])c(-c2ccccc2N(c2ccc(-c3ccccc3)cc2)c2cccc(-c3cccc4ccccc34)c2)c2c(oc3c4c([2H])c([2H])c([2H])c([2H])c4c([2H])c([2H])c32)c1[2H] ; [2H]c1c([2H])c(-c2ccccc2N(c2ccc(-c3ccccc3)cc2)c2cccc(-c3ccc4ccccc4c3)c2)c2c(oc3c4c([2H])c([2H])c([2H])c([2H])c4c([2H])c([2H])c32)c1[2H]]

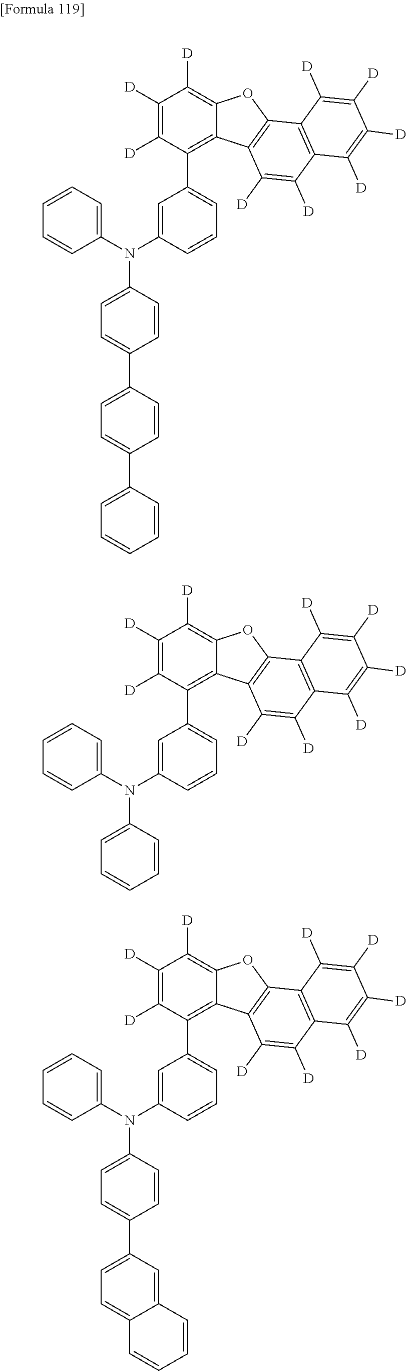 [SMILES: [2H]c1c([2H])c(-c2cccc(N(c3ccccc3)c3ccc(-c4ccc(-c5ccccc5)cc4)cc3)c2)c2c(oc3c4c([2H])c([2H])c([2H])c([2H])c4c([2H])c([2H])c32)c1[2H] ; [2H]c1c([2H])c(-c2cccc(N(c3ccccc3)c3ccccc3)c2)c2c(oc3c4c([2H])c([2H])c([2H])c([2H])c4c([2H])c([2H])c32)c1[2H] ; [2H]c1c([2H])c(-c2cccc(N(c3ccccc3)c3ccc(-c4ccc5ccccc5c4)cc3)c2)c2c(oc3c4c([2H])c([2H])c([2H])c([2H])c4c([2H])c([2H])c32)c1[2H]]

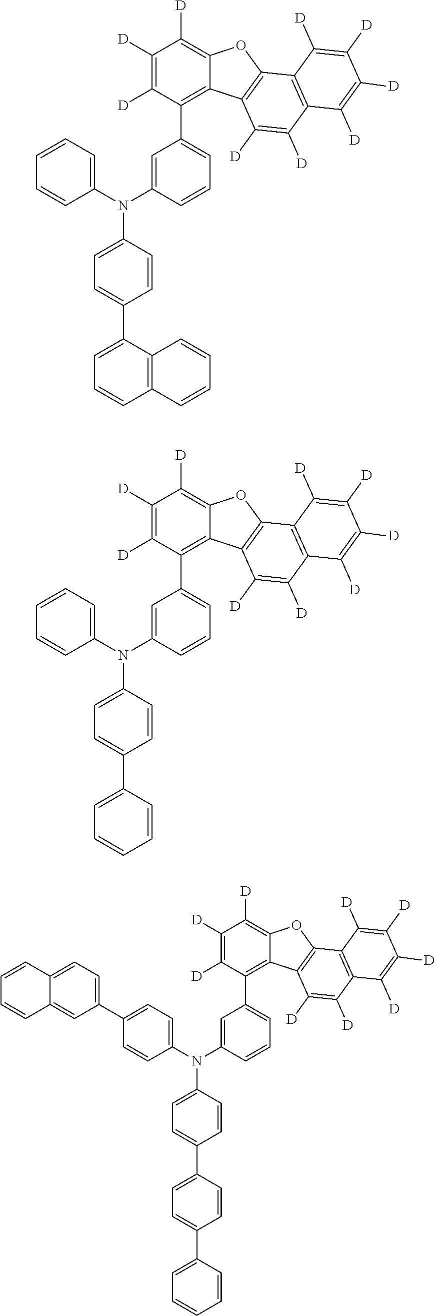 [SMILES: [2H]c1c([2H])c(-c2cccc(N(c3ccccc3)c3ccc(-c4cccc5ccccc45)cc3)c2)c2c(oc3c4c([2H])c([2H])c([2H])c([2H])c4c([2H])c([2H])c32)c1[2H] ; [2H]c1c([2H])c(-c2cccc(N(c3ccccc3)c3ccc(-c4ccccc4)cc3)c2)c2c(oc3c4c([2H])c([2H])c([2H])c([2H])c4c([2H])c([2H])c32)c1[2H] ; [2H]c1c([2H])c(-c2cccc(N(c3ccc(-c4ccc(-c5ccccc5)cc4)cc3)c3ccc(-c4ccc5ccccc5c4)cc3)c2)c2c(oc3c4c([2H])c([2H])c([2H])c([2H])c4c([2H])c([2H])c32)c1[2H]]

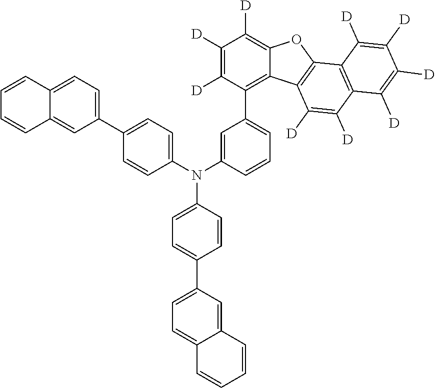 [SMILES: [2H]c1c([2H])c(-c2cccc(N(c3ccc(-c4ccc5ccccc5c4)cc3)c3ccc(-c4ccc5ccccc5c4)cc3)c2)c2c(oc3c4c([2H])c([2H])c([2H])c([2H])c4c([2H])c([2H])c32)c1[2H]]

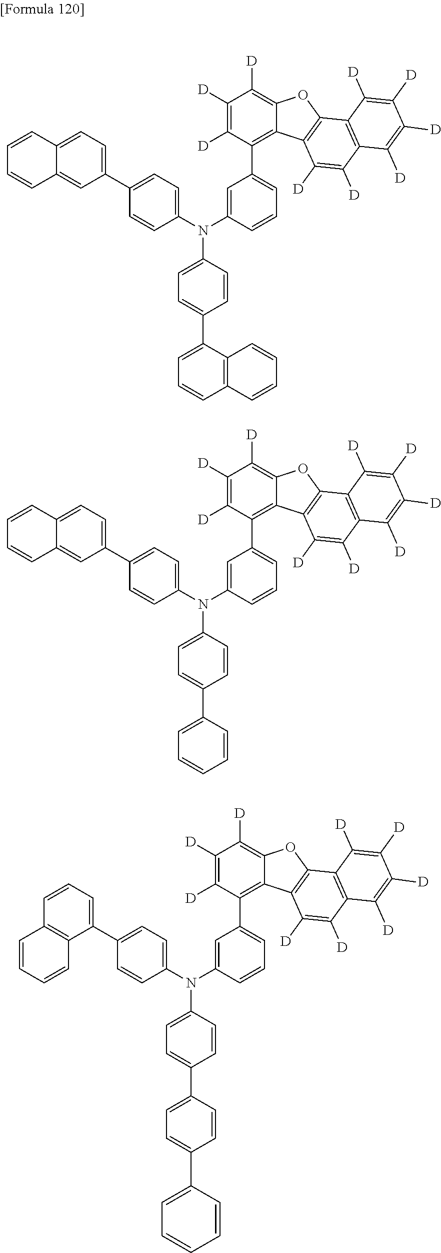

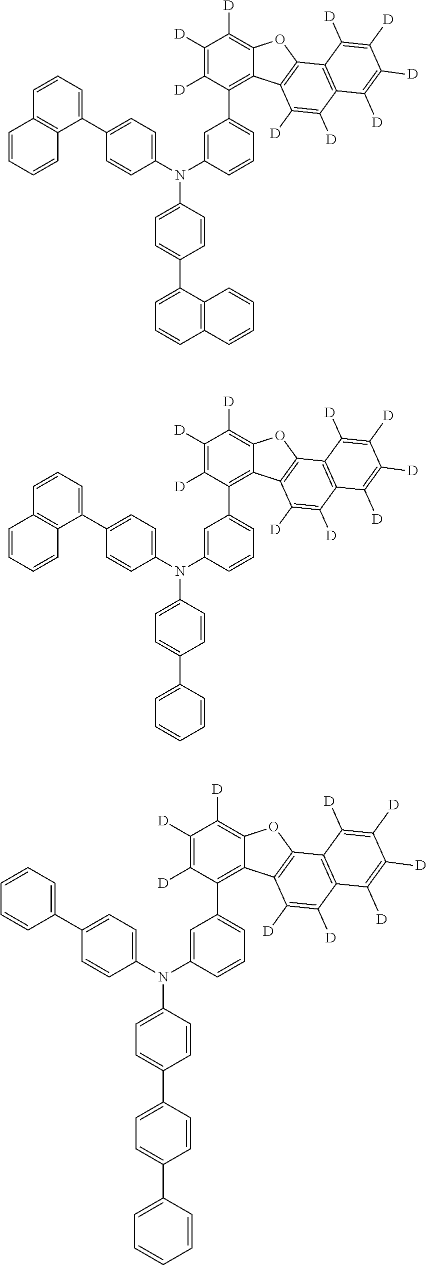 [SMILES: [2H]c1c([2H])c(-c2cccc(N(c3ccc(-c4cccc5ccccc45)cc3)c3ccc(-c4cccc5ccccc45)cc3)c2)c2c(oc3c4c([2H])c([2H])c([2H])c([2H])c4c([2H])c([2H])c32)c1[2H] ; [2H]c1c([2H])c(-c2cccc(N(c3ccc(-c4ccccc4)cc3)c3ccc(-c4cccc5ccccc45)cc3)c2)c2c(oc3c4c([2H])c([2H])c([2H])c([2H])c4c([2H])c([2H])c32)c1[2H] ; [2H]c1c([2H])c(-c2cccc(N(c3ccc(-c4ccccc4)cc3)c3ccc(-c4ccc(-c5ccccc5)cc4)cc3)c2)c2c(oc3c4c([2H])c([2H])c([2H])c([2H])c4c([2H])c([2H])c32)c1[2H]]

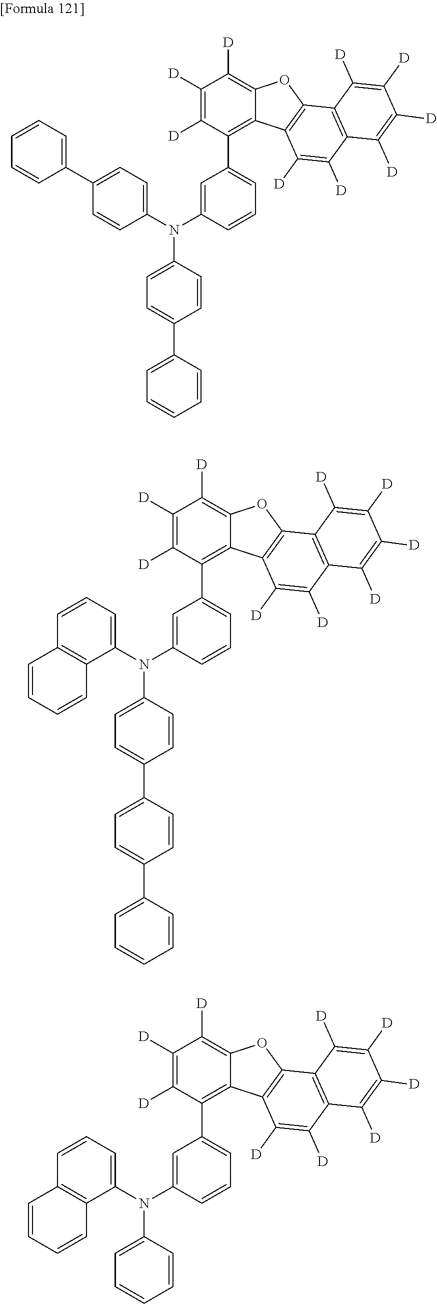 [SMILES: [2H]c1c([2H])c(-c2cccc(N(c3ccc(-c4ccccc4)cc3)c3ccc(-c4ccccc4)cc3)c2)c2c(oc3c4c([2H])c([2H])c([2H])c([2H])c4c([2H])c([2H])c32)c1[2H] ; [2H]c1c([2H])c(-c2cccc(N(c3ccc(-c4ccc(-c5ccccc5)cc4)cc3)c3cccc4ccccc34)c2)c2c(oc3c4c([2H])c([2H])c([2H])c([2H])c4c([2H])c([2H])c32)c1[2H] ; [2H]c1c([2H])c(-c2cccc(N(c3ccccc3)c3cccc4ccccc34)c2)c2c(oc3c4c([2H])c([2H])c([2H])c([2H])c4c([2H])c([2H])c32)c1[2H]]

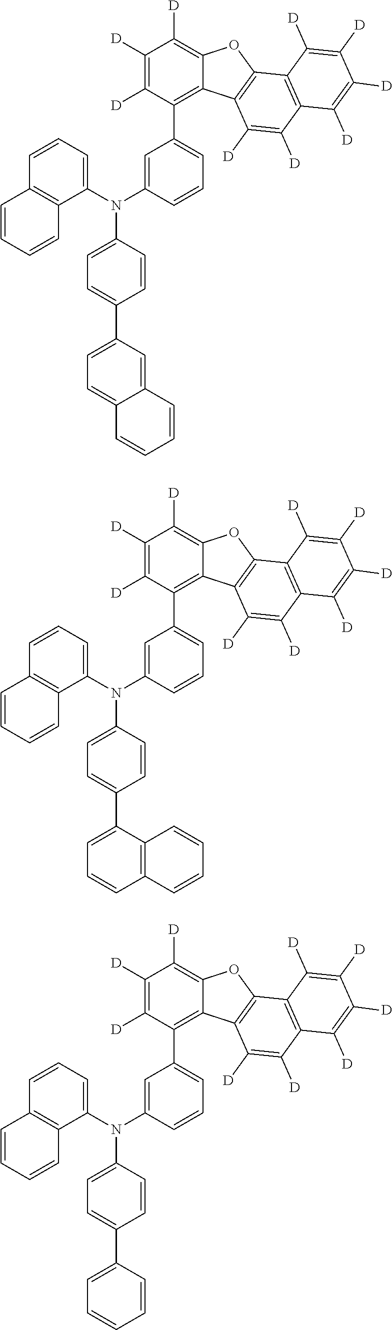

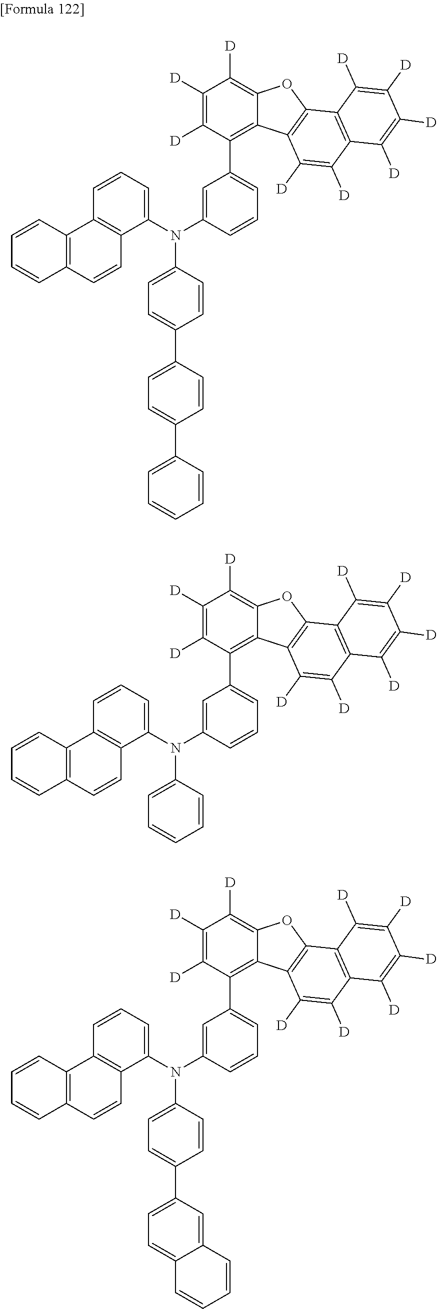 [SMILES: [2H]c1c([2H])c(-c2cccc(N(c3ccc(-c4ccc(-c5ccccc5)cc4)cc3)c3cccc4c3ccc3ccccc34)c2)c2c(oc3c4c([2H])c([2H])c([2H])c([2H])c4c([2H])c([2H])c32)c1[2H] ; [2H]c1c([2H])c(-c2cccc(N(c3ccccc3)c3cccc4c3ccc3ccccc34)c2)c2c(oc3c4c([2H])c([2H])c([2H])c([2H])c4c([2H])c([2H])c32)c1[2H] ; [2H]c1c([2H])c(-c2cccc(N(c3ccc(-c4ccc5ccccc5c4)cc3)c3cccc4c3ccc3ccccc34)c2)c2c(oc3c4c([2H])c([2H])c([2H])c([2H])c4c([2H])c([2H])c32)c1[2H]]

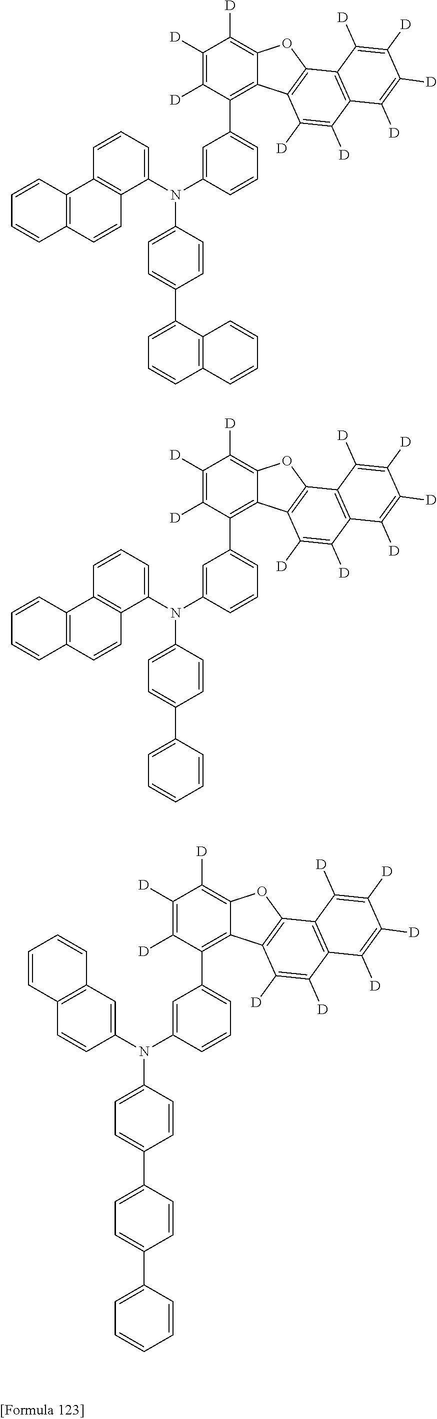

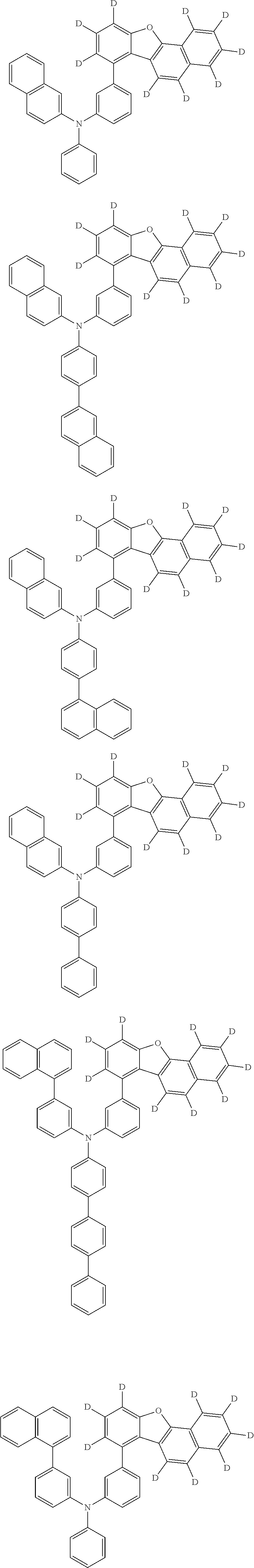

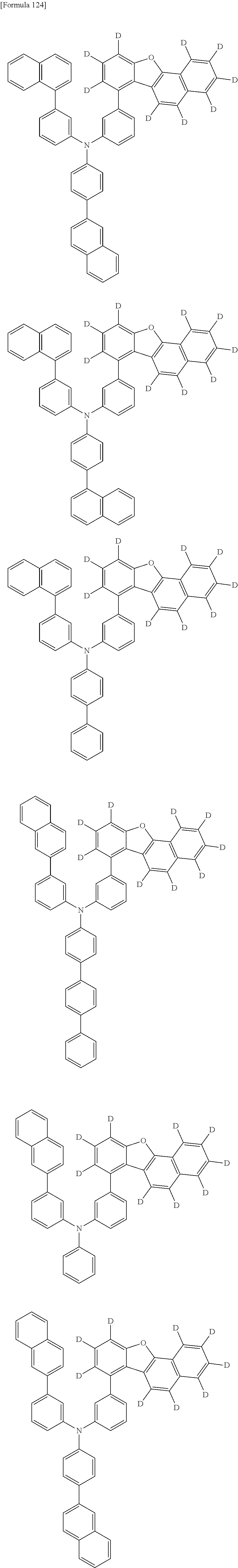

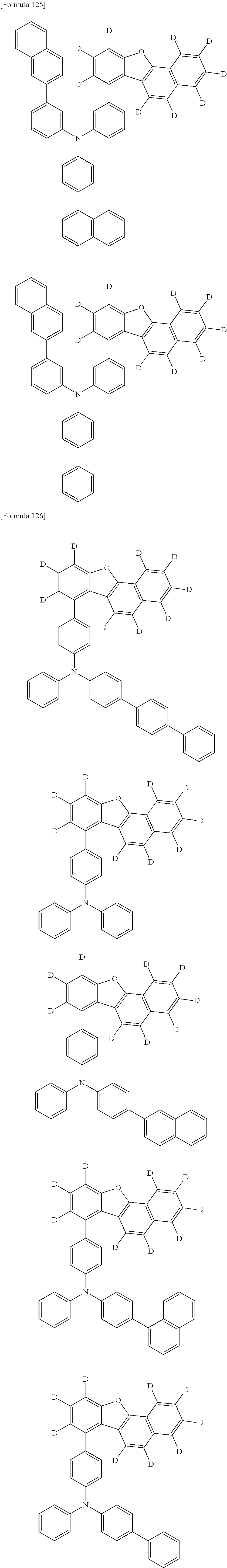

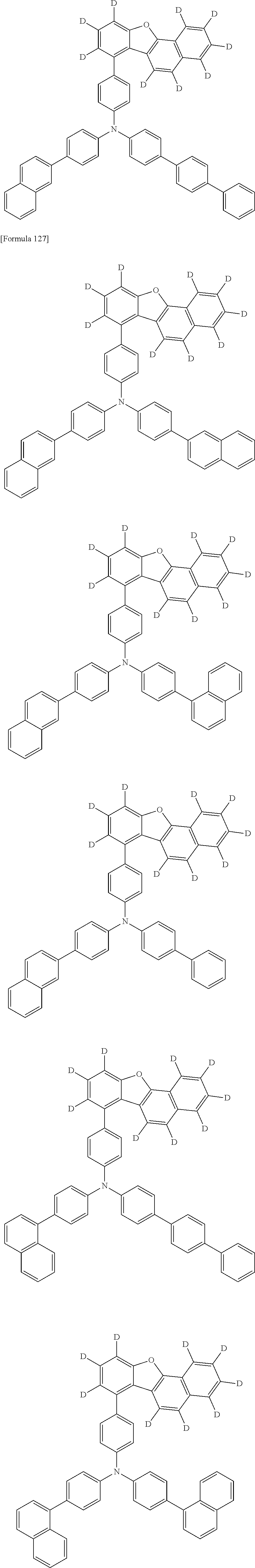

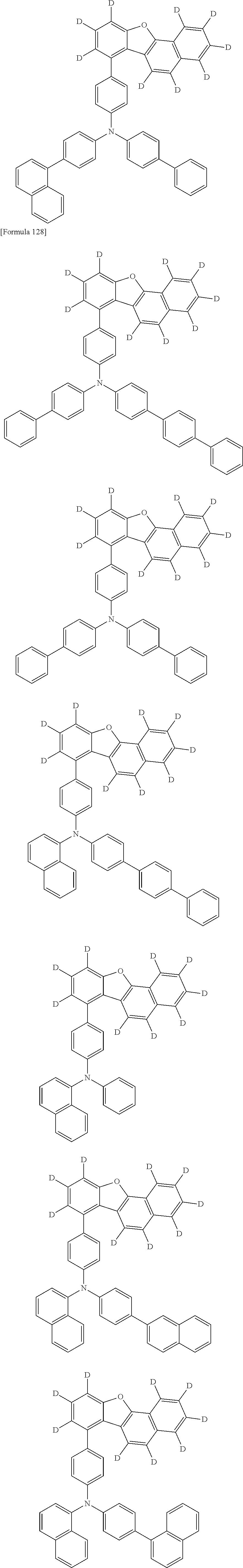

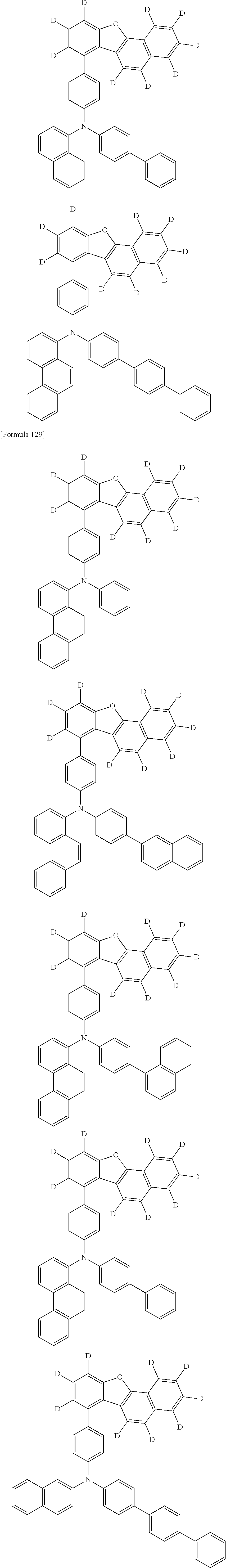

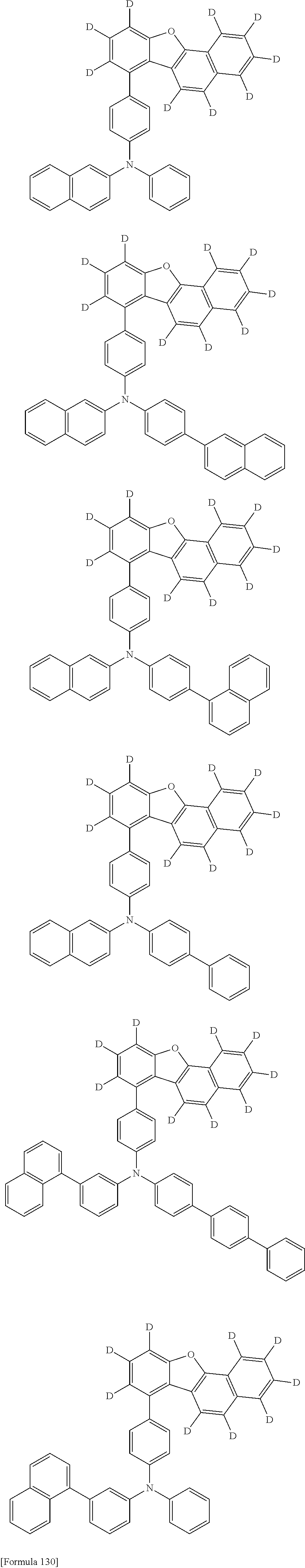

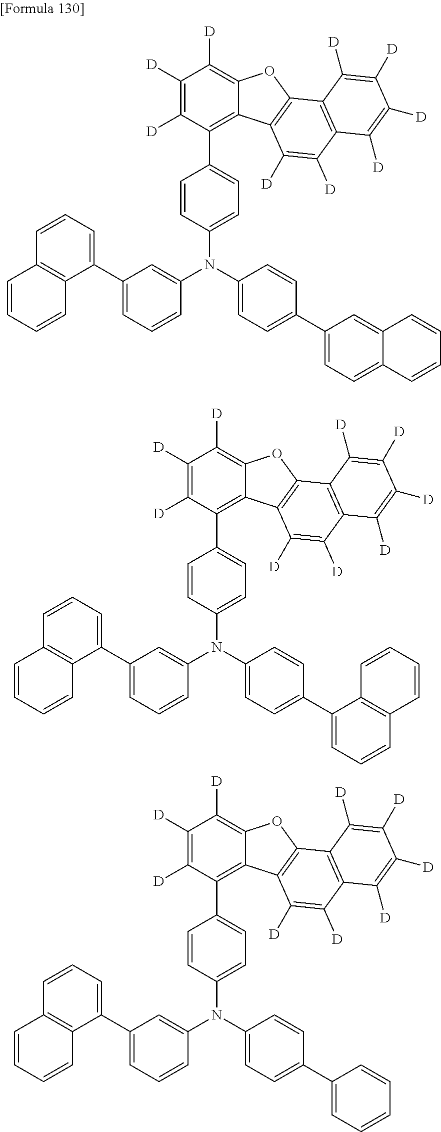

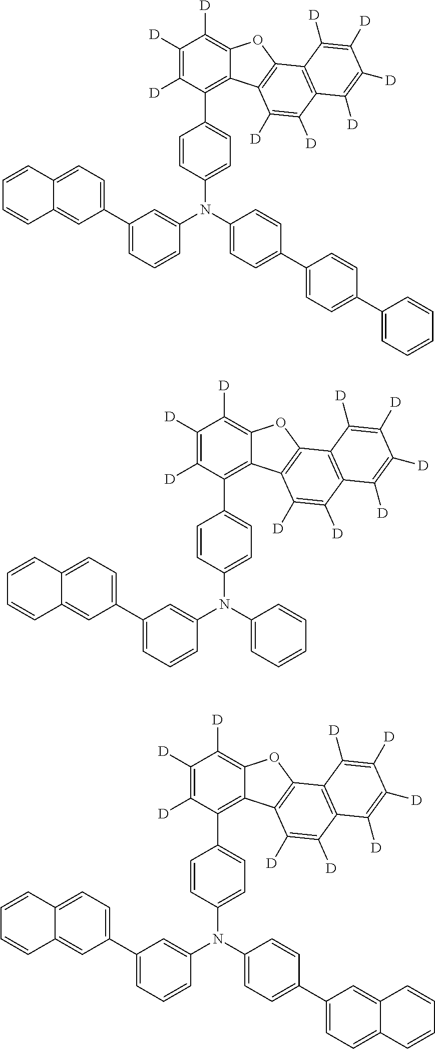 [SMILES: [2H]c1c([2H])c(-c2ccc(N(c3ccc(-c4ccc(-c5ccccc5)cc4)cc3)c3cccc(-c4ccc5ccccc5c4)c3)cc2)c2c(oc3c4c([2H])c([2H])c([2H])c([2H])c4c([2H])c([2H])c32)c1[2H] ; [2H]c1c([2H])c(-c2ccc(N(c3ccccc3)c3cccc(-c4ccc5ccccc5c4)c3)cc2)c2c(oc3c4c([2H])c([2H])c([2H])c([2H])c4c([2H])c([2H])c32)c1[2H] ; [2H]c1c([2H])c(-c2ccc(N(c3ccc(-c4ccc5ccccc5c4)cc3)c3cccc(-c4ccc5ccccc5c4)c3)cc2)c2c(oc3c4c([2H])c([2H])c([2H])c([2H])c4c([2H])c([2H])c32)c1[2H]]

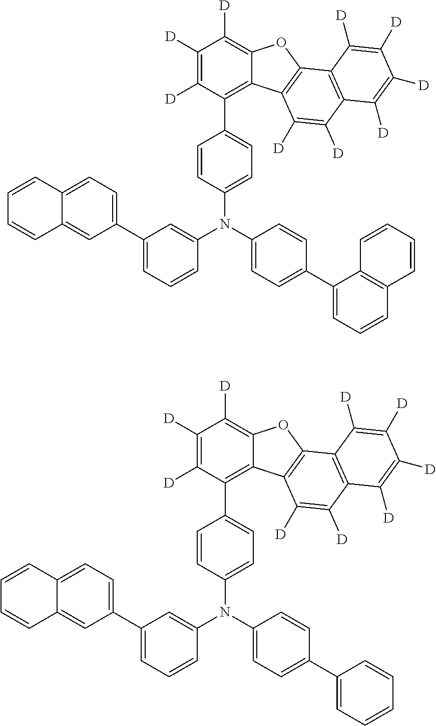 [SMILES: [2H]c1c([2H])c(-c2ccc(N(c3ccc(-c4cccc5ccccc45)cc3)c3cccc(-c4ccc5ccccc5c4)c3)cc2)c2c(oc3c4c([2H])c([2H])c([2H])c([2H])c4c([2H])c([2H])c32)c1[2H] ; [2H]c1c([2H])c(-c2ccc(N(c3ccc(-c4ccccc4)cc3)c3cccc(-c4ccc5ccccc5c4)c3)cc2)c2c(oc3c4c([2H])c([2H])c([2H])c([2H])c4c([2H])c([2H])c32)c1[2H]]

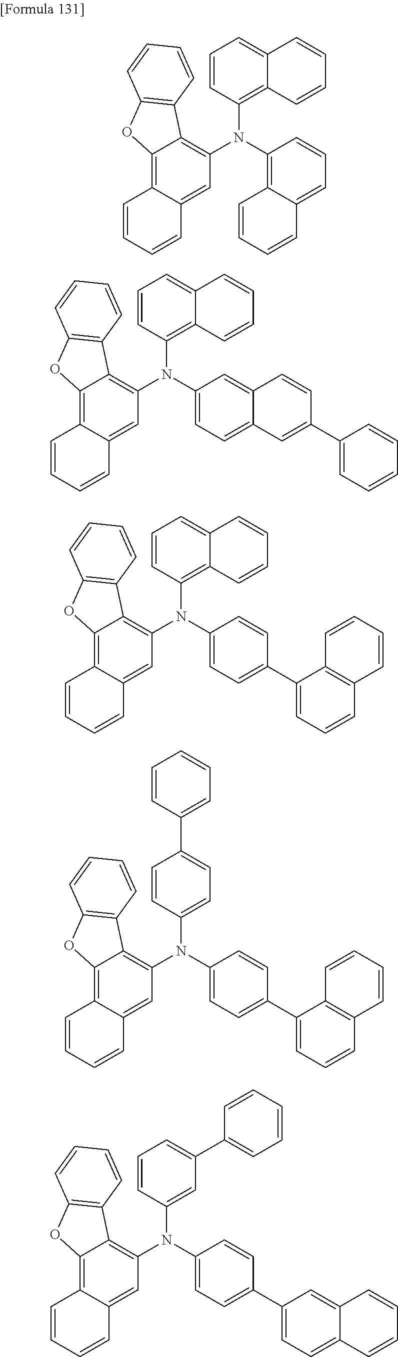

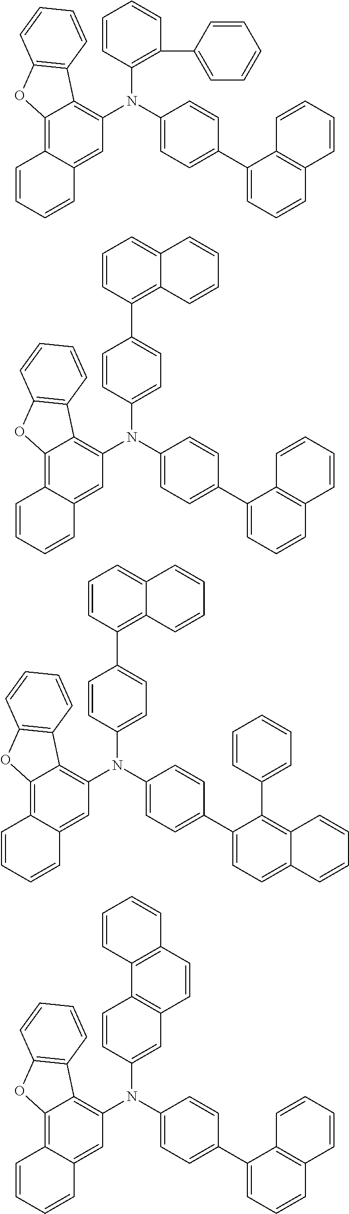 [SMILES: c1ccc(-c2ccccc2N(c2ccc(-c3cccc4ccccc34)cc2)c2cc3ccccc3c3oc4ccccc4c23)cc1 ; c1ccc2c(-c3ccc(N(c4ccc(-c5cccc6ccccc56)cc4)c4cc5ccccc5c5oc6ccccc6c45)cc3)cccc2c1 ; c1ccc(-c2c(-c3ccc(N(c4ccc(-c5cccc6ccccc56)cc4)c4cc5ccccc5c5oc6ccccc6c45)cc3)ccc3ccccc23)cc1 ; c1ccc2c(-c3ccc(N(c4ccc5c(ccc6ccccc65)c4)c4cc5ccccc5c5oc6ccccc6c45)cc3)cccc2c1]

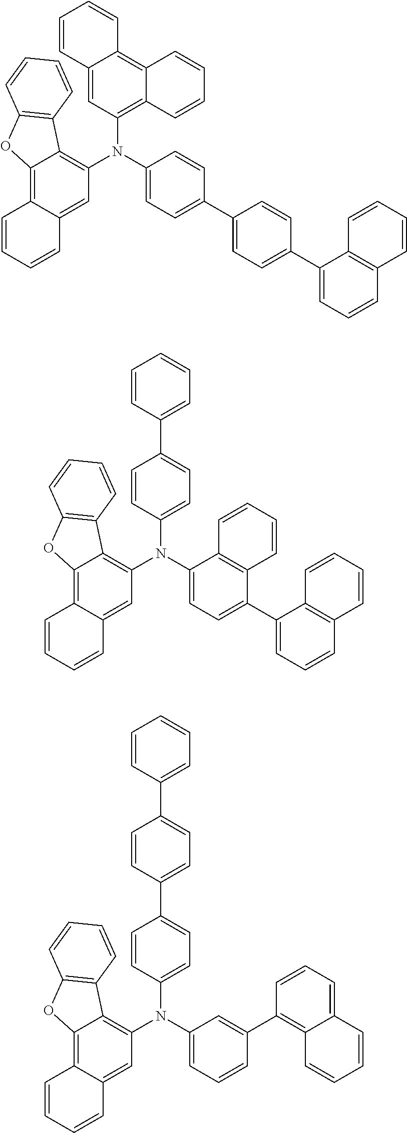 [SMILES: c1ccc2c(-c3ccc(-c4ccc(N(c5cc6ccccc6c6ccccc56)c5cc6ccccc6c6oc7ccccc7c56)cc4)cc3)cccc2c1 ; c1ccc(-c2ccc(N(c3ccc(-c4cccc5ccccc45)c4ccccc34)c3cc4ccccc4c4oc5ccccc5c34)cc2)cc1 ; c1ccc(-c2ccc(-c3ccc(N(c4cccc(-c5cccc6ccccc56)c4)c4cc5ccccc5c5oc6ccccc6c45)cc3)cc2)cc1]

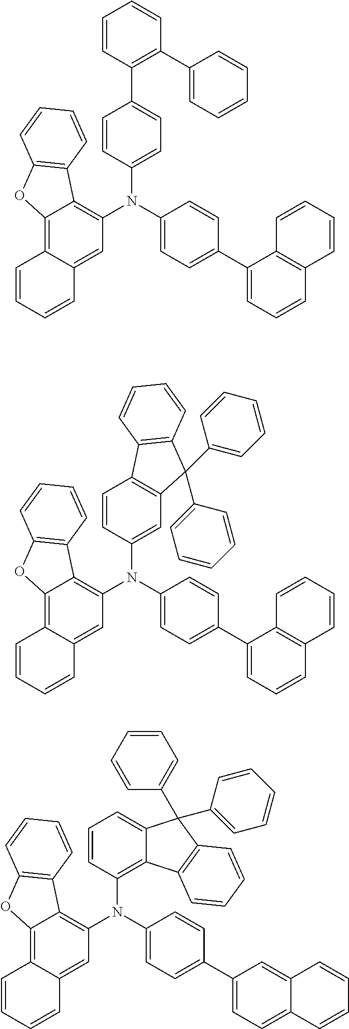 [SMILES: c1ccc(-c2ccccc2-c2ccc(N(c3ccc(-c4cccc5ccccc45)cc3)c3cc4ccccc4c4oc5ccccc5c34)cc2)cc1 ; c1ccc(C2(c3ccccc3)c3ccccc3-c3ccc(N(c4ccc(-c5cccc6ccccc56)cc4)c4cc5ccccc5c5oc6ccccc6c45)cc32)cc1 ; c1ccc(C2(c3ccccc3)c3ccccc3-c3c(N(c4ccc(-c5ccc6ccccc6c5)cc4)c4cc5ccccc5c5oc6ccccc6c45)cccc32)cc1]

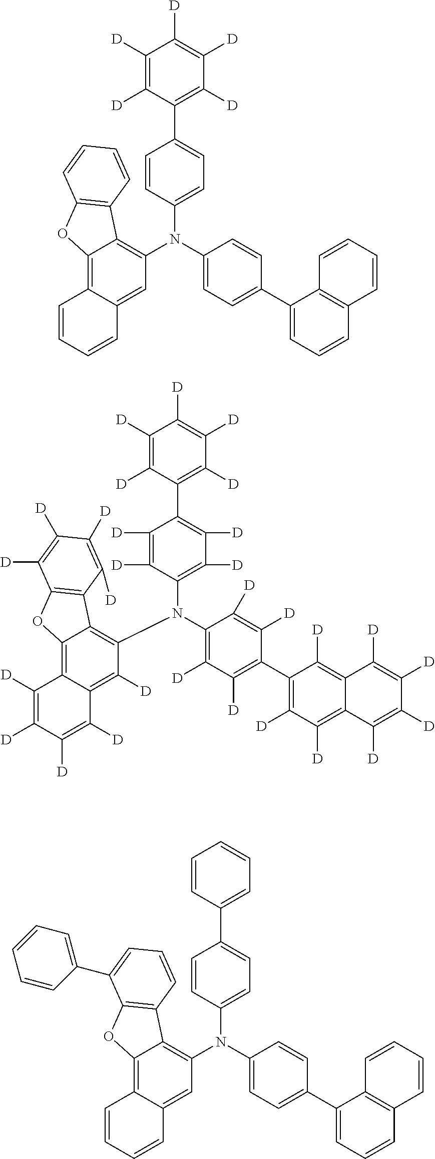 [SMILES: [2H]c1c([2H])c([2H])c(-c2ccc(N(c3ccc(-c4cccc5ccccc45)cc3)c3cc4ccccc4c4oc5ccccc5c34)cc2)c([2H])c1[2H] ; [2H]c1c([2H])c([2H])c(-c2c([2H])c([2H])c(N(c3c([2H])c([2H])c(-c4c([2H])c([2H])c5c([2H])c([2H])c([2H])c([2H])c5c4[2H])c([2H])c3[2H])c3c([2H])c4c([2H])c([2H])c([2H])c([2H])c4c4oc5c([2H])c([2H])c([2H])c([2H])c5c34)c([2H])c2[2H])c([2H])c1[2H] ; c1ccc(-c2ccc(N(c3ccc(-c4cccc5ccccc45)cc3)c3cc4ccccc4c4oc5c(-c6ccccc6)cccc5c34)cc2)cc1]

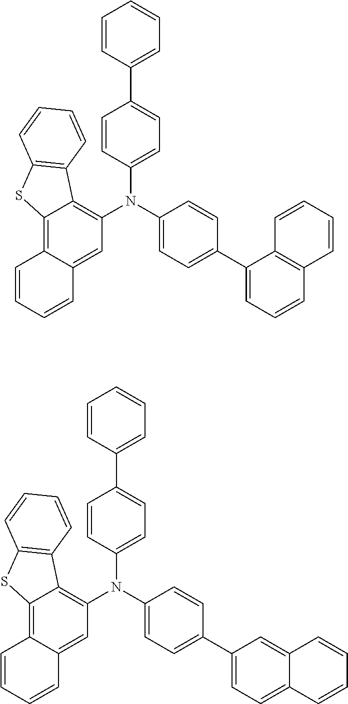 [SMILES: c1ccc(-c2ccc(N(c3ccc(-c4cccc5ccccc45)cc3)c3cc4ccccc4c4sc5ccccc5c34)cc2)cc1 ; c1ccc(-c2ccc(N(c3ccc(-c4ccc5ccccc5c4)cc3)c3cc4ccccc4c4sc5ccccc5c34)cc2)cc1]

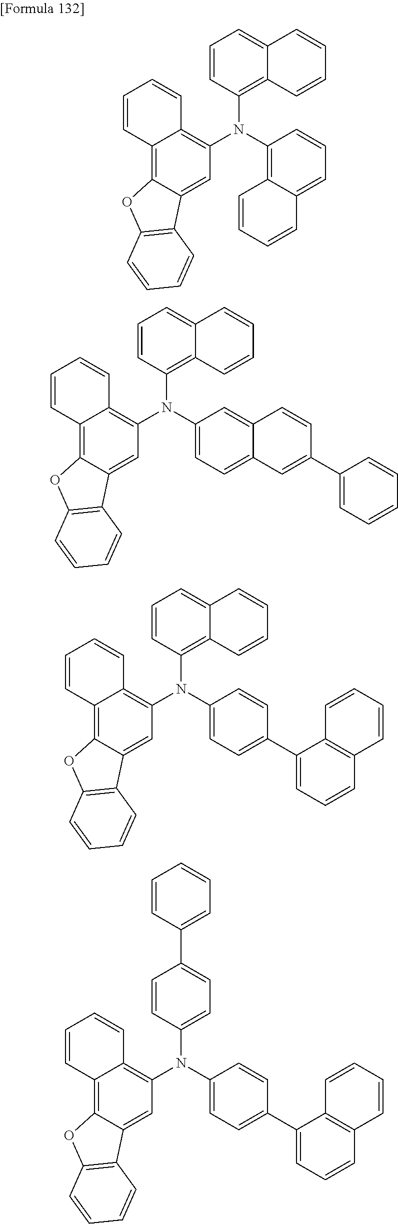

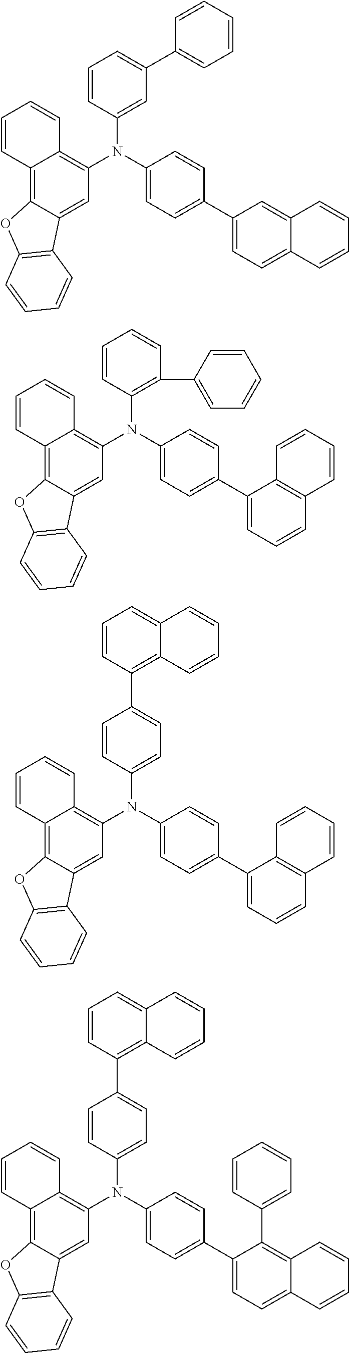 [SMILES: c1ccc(-c2cccc(N(c3ccc(-c4ccc5ccccc5c4)cc3)c3cc4c5ccccc5oc4c4ccccc34)c2)cc1 ; c1ccc(-c2ccccc2N(c2ccc(-c3cccc4ccccc34)cc2)c2cc3c4ccccc4oc3c3ccccc23)cc1 ; c1ccc2c(-c3ccc(N(c4ccc(-c5cccc6ccccc56)cc4)c4cc5c6ccccc6oc5c5ccccc45)cc3)cccc2c1 ; c1ccc(-c2c(-c3ccc(N(c4ccc(-c5cccc6ccccc56)cc4)c4cc5c6ccccc6oc5c5ccccc45)cc3)ccc3ccccc23)cc1]

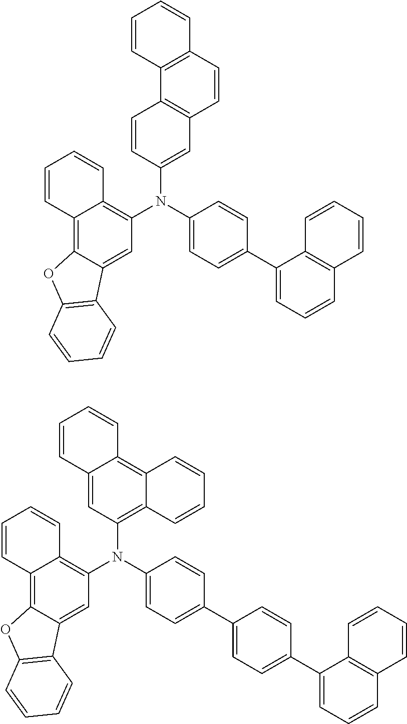 [SMILES: c1ccc2c(-c3ccc(N(c4ccc5c(ccc6ccccc65)c4)c4cc5c6ccccc6oc5c5ccccc45)cc3)cccc2c1 ; c1ccc2c(-c3ccc(-c4ccc(N(c5cc6ccccc6c6ccccc56)c5cc6c7ccccc7oc6c6ccccc56)cc4)cc3)cccc2c1]

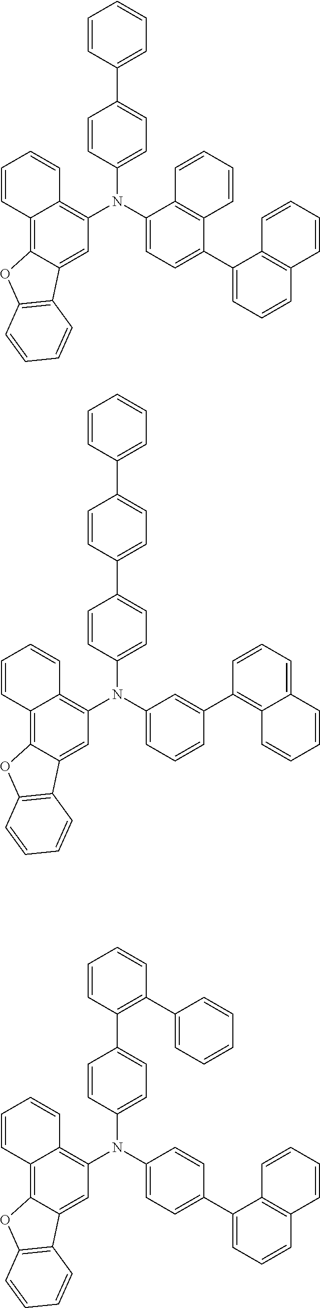 [SMILES: c1ccc(-c2ccc(N(c3ccc(-c4cccc5ccccc45)c4ccccc34)c3cc4c5ccccc5oc4c4ccccc34)cc2)cc1 ; c1ccc(-c2ccc(-c3ccc(N(c4cccc(-c5cccc6ccccc56)c4)c4cc5c6ccccc6oc5c5ccccc45)cc3)cc2)cc1 ; c1ccc(-c2ccccc2-c2ccc(N(c3ccc(-c4cccc5ccccc45)cc3)c3cc4c5ccccc5oc4c4ccccc34)cc2)cc1]

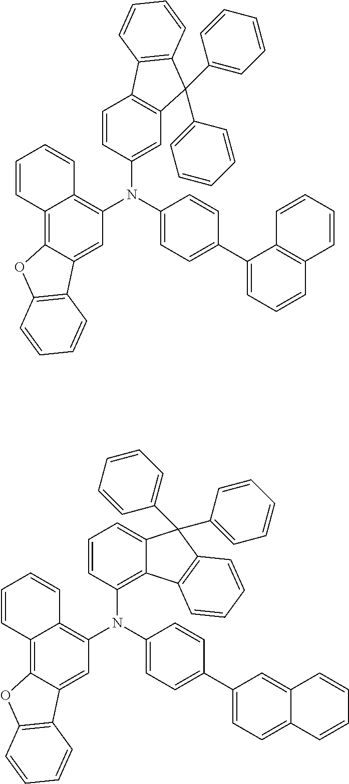 [SMILES: c1ccc(C2(c3ccccc3)c3ccccc3-c3ccc(N(c4ccc(-c5cccc6ccccc56)cc4)c4cc5c6ccccc6oc5c5ccccc45)cc32)cc1 ; c1ccc(C2(c3ccccc3)c3ccccc3-c3c(N(c4ccc(-c5ccc6ccccc6c5)cc4)c4cc5c6ccccc6oc5c5ccccc45)cccc32)cc1]

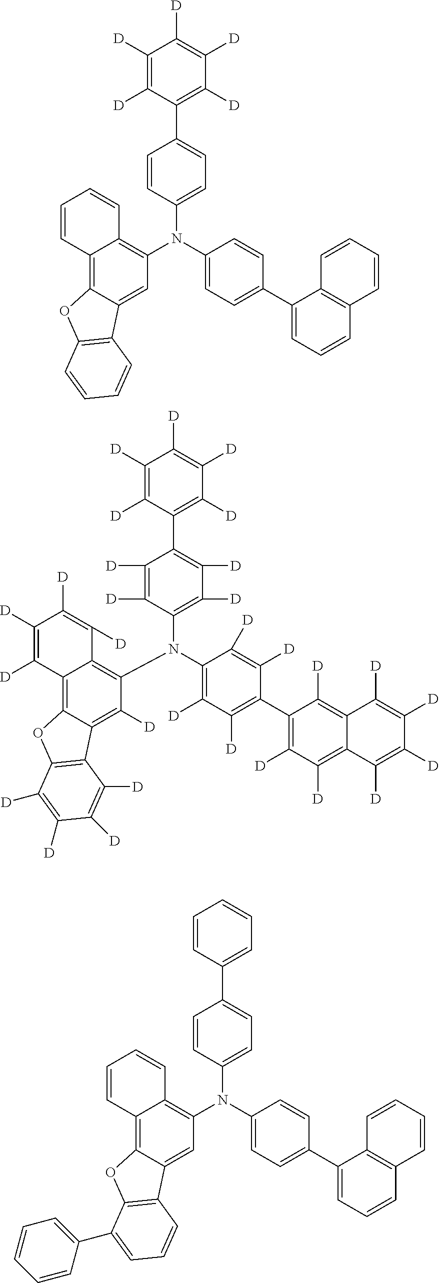 [SMILES: [2H]c1c([2H])c([2H])c(-c2ccc(N(c3ccc(-c4cccc5ccccc45)cc3)c3cc4c5ccccc5oc4c4ccccc34)cc2)c([2H])c1[2H] ; [2H]c1c([2H])c([2H])c(-c2c([2H])c([2H])c(N(c3c([2H])c([2H])c(-c4c([2H])c([2H])c5c([2H])c([2H])c([2H])c([2H])c5c4[2H])c([2H])c3[2H])c3c([2H])c4c(oc5c([2H])c([2H])c([2H])c([2H])c54)c4c([2H])c([2H])c([2H])c([2H])c34)c([2H])c2[2H])c([2H])c1[2H] ; c1ccc(-c2ccc(N(c3ccc(-c4cccc5ccccc45)cc3)c3cc4c5cccc(-c6ccccc6)c5oc4c4ccccc34)cc2)cc1]

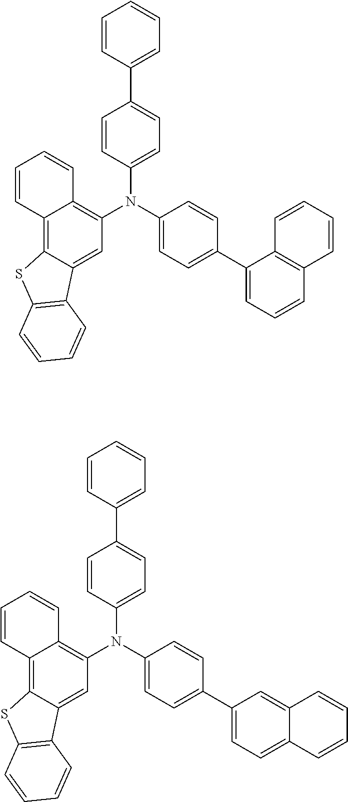 [SMILES: c1ccc(-c2ccc(N(c3ccc(-c4cccc5ccccc45)cc3)c3cc4c5ccccc5sc4c4ccccc34)cc2)cc1 ; c1ccc(-c2ccc(N(c3ccc(-c4ccc5ccccc5c4)cc3)c3cc4c5ccccc5sc4c4ccccc34)cc2)cc1]

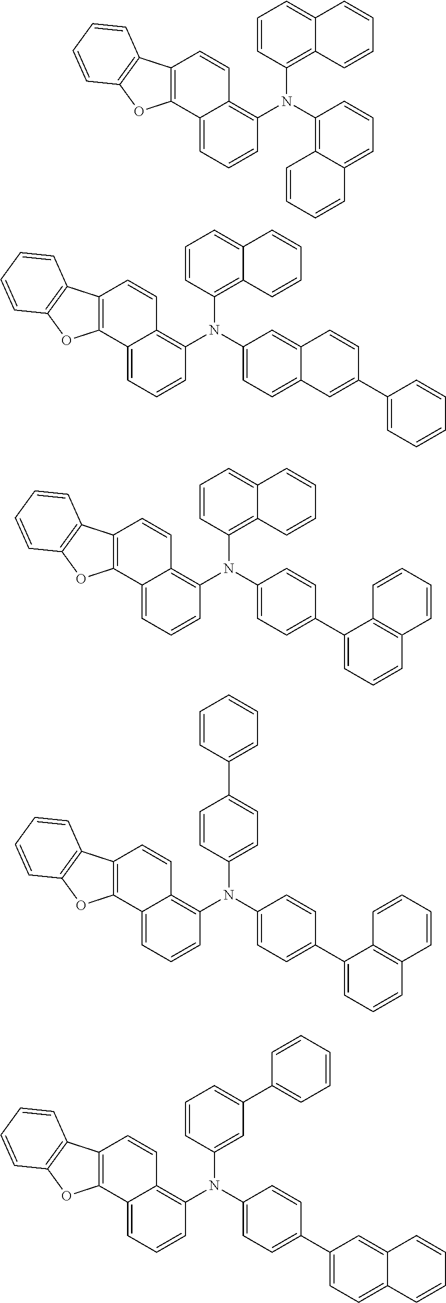

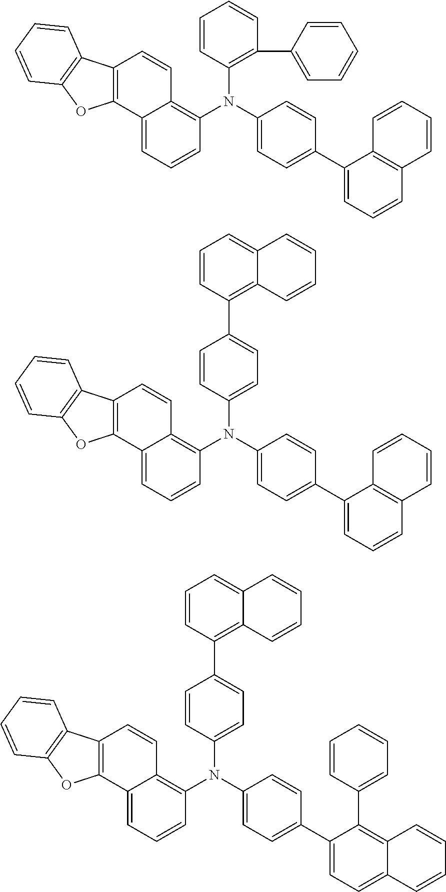 [SMILES: c1ccc(-c2ccccc2N(c2ccc(-c3cccc4ccccc34)cc2)c2cccc3c2ccc2c4ccccc4oc32)cc1 ; c1ccc2c(-c3ccc(N(c4ccc(-c5cccc6ccccc56)cc4)c4cccc5c4ccc4c6ccccc6oc54)cc3)cccc2c1 ; c1ccc(-c2c(-c3ccc(N(c4ccc(-c5cccc6ccccc56)cc4)c4cccc5c4ccc4c6ccccc6oc54)cc3)ccc3ccccc23)cc1]

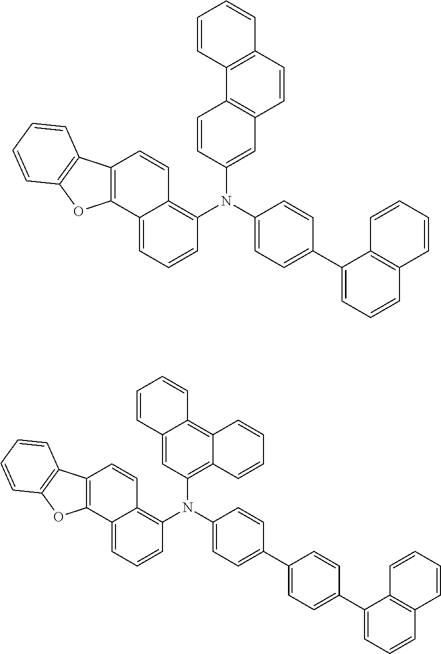 [SMILES: c1ccc2c(-c3ccc(N(c4ccc5c(ccc6ccccc65)c4)c4cccc5c4ccc4c6ccccc6oc54)cc3)cccc2c1 ; c1ccc2c(-c3ccc(-c4ccc(N(c5cc6ccccc6c6ccccc56)c5cccc6c5ccc5c7ccccc7oc65)cc4)cc3)cccc2c1]

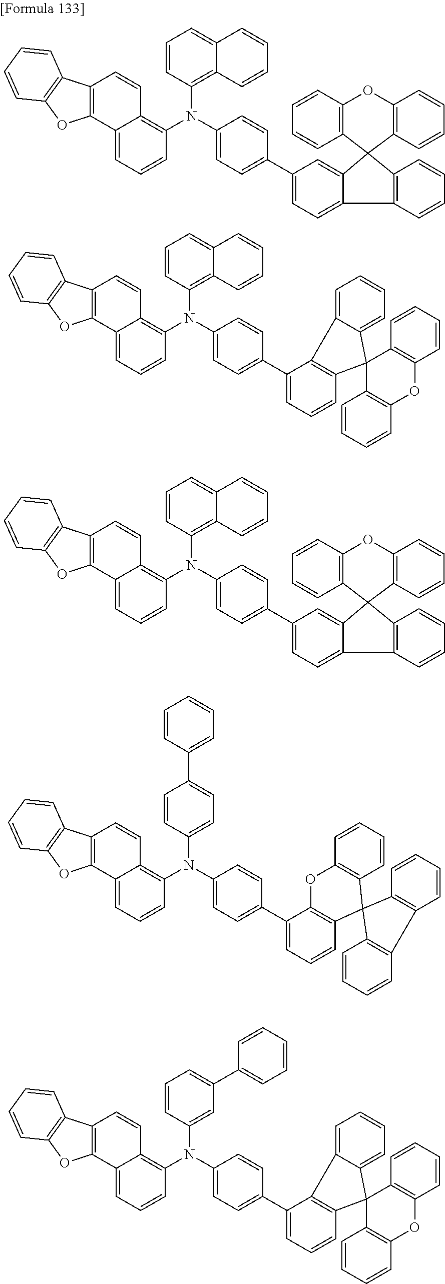 [SMILES: c1ccc2c(c1)Oc1ccccc1C21c2ccccc2-c2ccc(-c3ccc(N(c4cccc5ccccc45)c4cccc5c4ccc4c6ccccc6oc54)cc3)cc21 ; c1ccc2c(c1)Oc1ccccc1C21c2ccccc2-c2c(-c3ccc(N(c4cccc5ccccc45)c4cccc5c4ccc4c6ccccc6oc54)cc3)cccc21 ; c1ccc2c(c1)Oc1ccccc1C21c2ccccc2-c2ccc(-c3ccc(N(c4cccc5ccccc45)c4cccc5c4ccc4c6ccccc6oc54)cc3)cc21 ; c1ccc(-c2ccc(N(c3ccc(-c4cccc5c4Oc4ccccc4C54c5ccccc5-c5ccccc54)cc3)c3cccc4c3ccc3c5ccccc5oc43)cc2)cc1 ; c1ccc(-c2cccc(N(c3ccc(-c4cccc5c4-c4ccccc4C54c5ccccc5Oc5ccccc54)cc3)c3cccc4c3ccc3c5ccccc5oc43)c2)cc1]

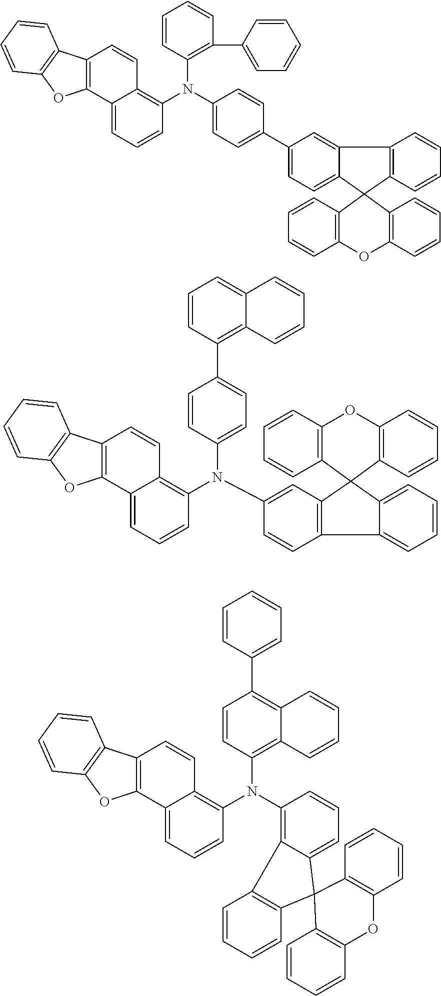

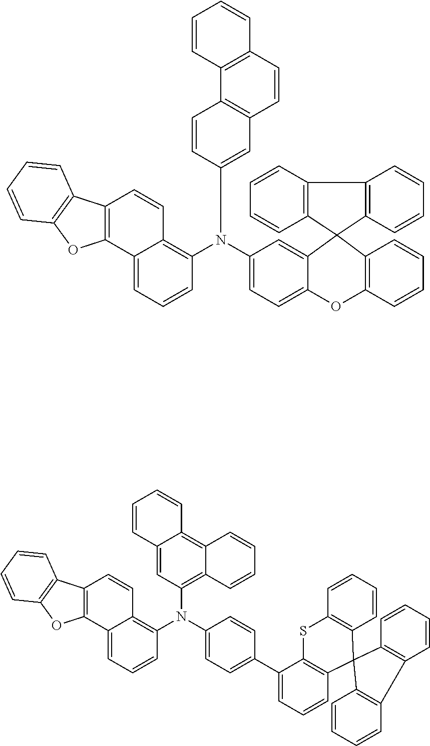 [SMILES: c1ccc2c(c1)Oc1ccc(N(c3ccc4c(ccc5ccccc54)c3)c3cccc4c3ccc3c5ccccc5oc43)cc1C21c2ccccc2-c2ccccc21 ; c1ccc2c(c1)Sc1c(-c3ccc(N(c4cc5ccccc5c5ccccc45)c4cccc5c4ccc4c6ccccc6oc54)cc3)cccc1C21c2ccccc2-c2ccccc21]

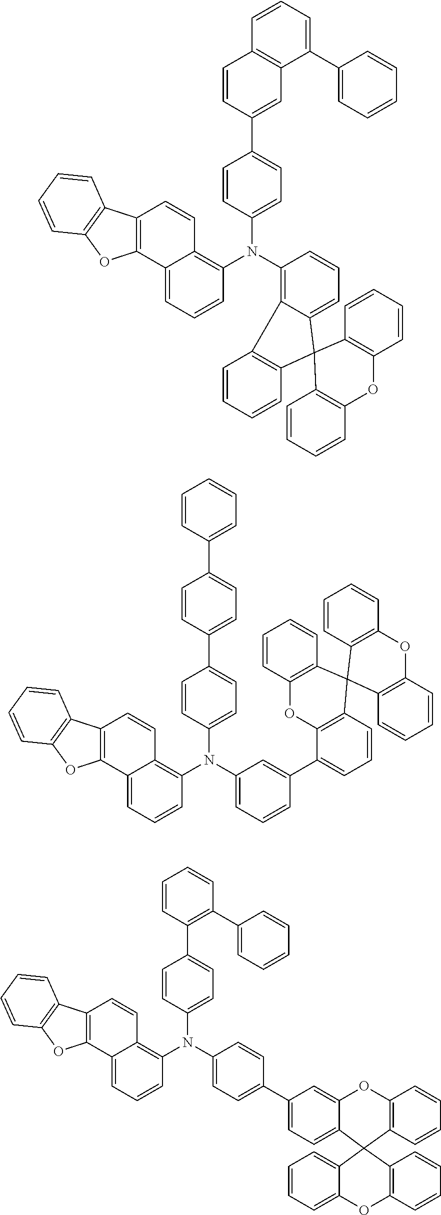 [SMILES: c1ccc(-c2cccc3ccc(-c4ccc(N(c5cccc6c5-c5ccccc5C65c6ccccc6Oc6ccccc65)c5cccc6c5ccc5c7ccccc7oc65)cc4)cc23)cc1 ; c1ccc(-c2ccc(-c3ccc(N(c4cccc(-c5cccc6c5Oc5ccccc5C65c6ccccc6Oc6ccccc65)c4)c4cccc5c4ccc4c6ccccc6oc54)cc3)cc2)cc1 ; c1ccc(-c2ccccc2-c2ccc(N(c3ccc(-c4ccc5c(c4)Oc4ccccc4C54c5ccccc5Oc5ccccc54)cc3)c3cccc4c3ccc3c5ccccc5oc43)cc2)cc1]

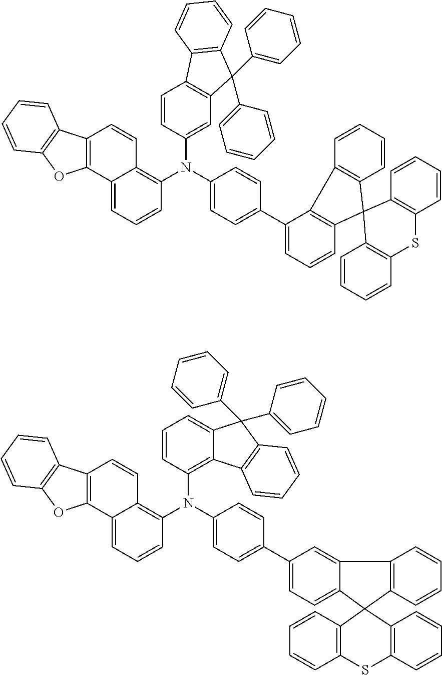 [SMILES: c1ccc(C2(c3ccccc3)c3ccccc3-c3ccc(N(c4ccc(-c5cccc6c5-c5ccccc5C65c6ccccc6Sc6ccccc65)cc4)c4cccc5c4ccc4c6ccccc6oc54)cc32)cc1 ; c1ccc(C2(c3ccccc3)c3ccccc3-c3c(N(c4ccc(-c5ccc6c(c5)-c5ccccc5C65c6ccccc6Sc6ccccc65)cc4)c4cccc5c4ccc4c6ccccc6oc54)cccc32)cc1]

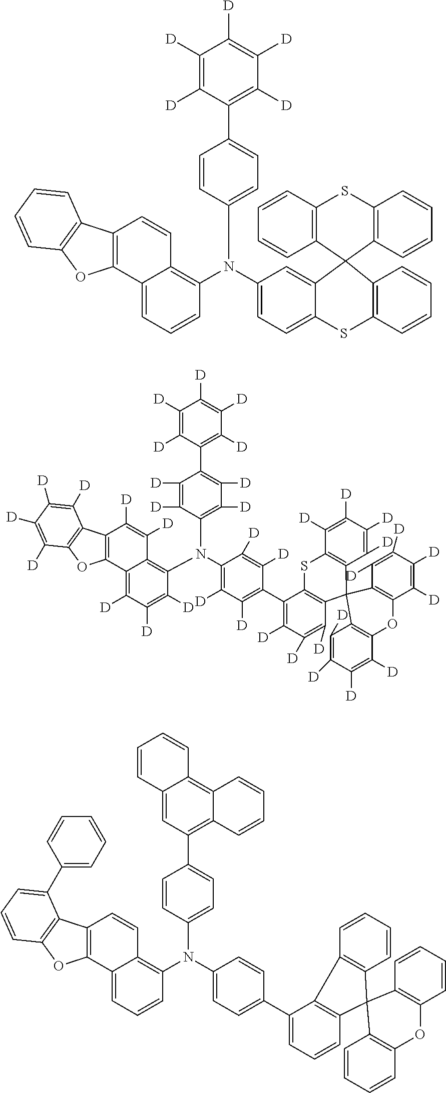

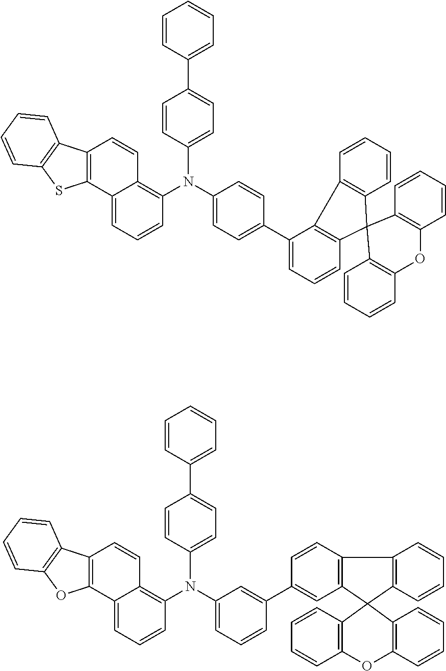 [SMILES: c1ccc(-c2ccc(N(c3ccc(-c4cccc5c4-c4ccccc4C54c5ccccc5Oc5ccccc54)cc3)c3cccc4c3ccc3c5ccccc5sc43)cc2)cc1 ; c1ccc(-c2ccc(N(c3cccc(-c4ccc5c(c4)C4(c6ccccc6Oc6ccccc64)c4ccccc4-5)c3)c3cccc4c3ccc3c5ccccc5oc43)cc2)cc1]

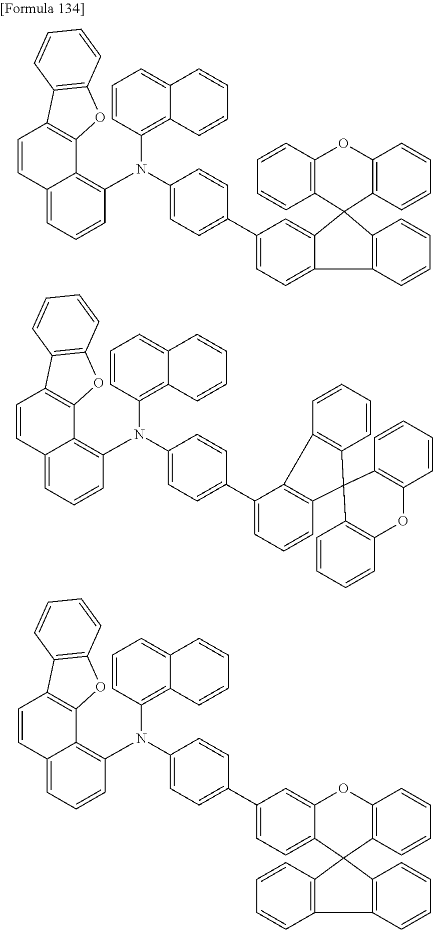 [SMILES: c1ccc2c(c1)Oc1ccccc1C21c2ccccc2-c2ccc(-c3ccc(N(c4cccc5ccccc45)c4cccc5ccc6c7ccccc7oc6c45)cc3)cc21 ; c1ccc2c(c1)Oc1ccccc1C21c2ccccc2-c2c(-c3ccc(N(c4cccc5ccccc45)c4cccc5ccc6c7ccccc7oc6c45)cc3)cccc21 ; c1ccc2c(c1)Oc1cc(-c3ccc(N(c4cccc5ccccc45)c4cccc5ccc6c7ccccc7oc6c45)cc3)ccc1C21c2ccccc2-c2ccccc21]

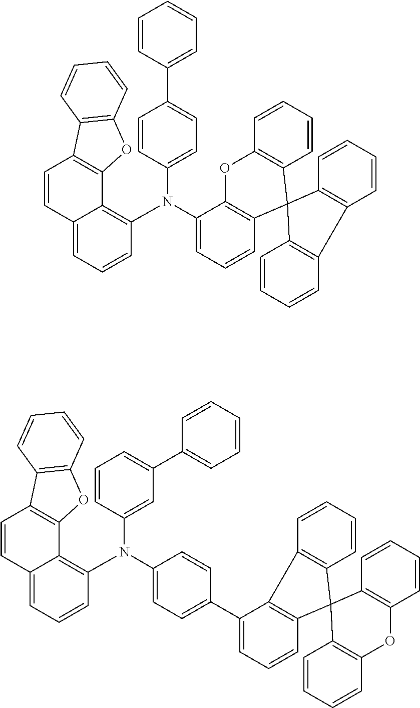 [SMILES: c1ccc(-c2ccc(N(c3cccc4c3Oc3ccccc3C43c4ccccc4-c4ccccc43)c3cccc4ccc5c6ccccc6oc5c34)cc2)cc1 ; c1ccc(-c2cccc(N(c3ccc(-c4cccc5c4-c4ccccc4C54c5ccccc5Oc5ccccc54)cc3)c3cccc4ccc5c6ccccc6oc5c34)c2)cc1]

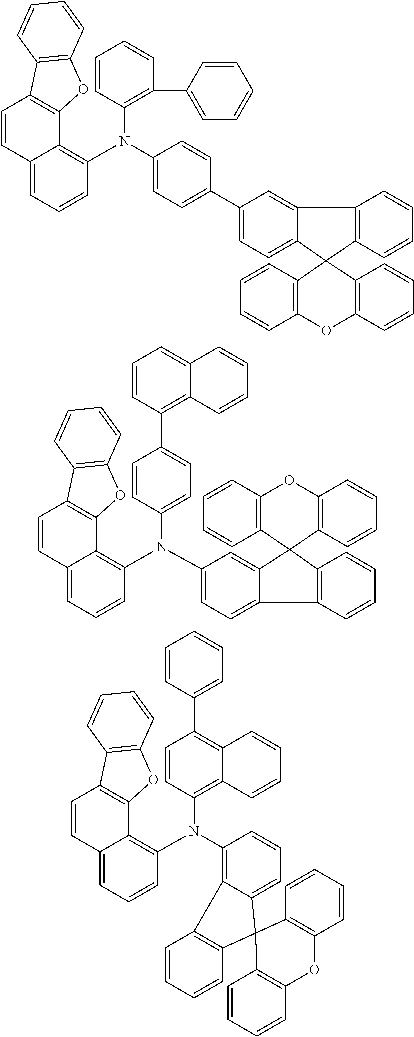

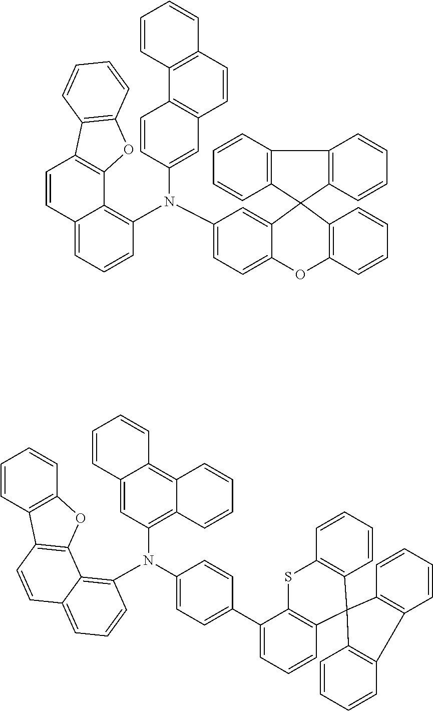 [SMILES: c1ccc2c(c1)Oc1ccc(N(c3ccc4c(ccc5ccccc54)c3)c3cccc4ccc5c6ccccc6oc5c34)cc1C21c2ccccc2-c2ccccc21 ; c1ccc2c(c1)Sc1c(-c3ccc(N(c4cc5ccccc5c5ccccc45)c4cccc5ccc6c7ccccc7oc6c45)cc3)cccc1C21c2ccccc2-c2ccccc21]

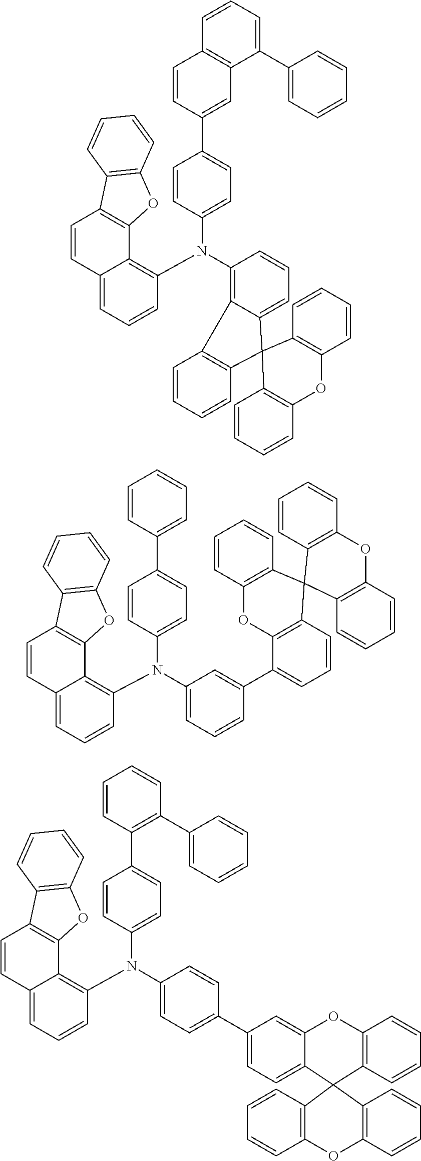

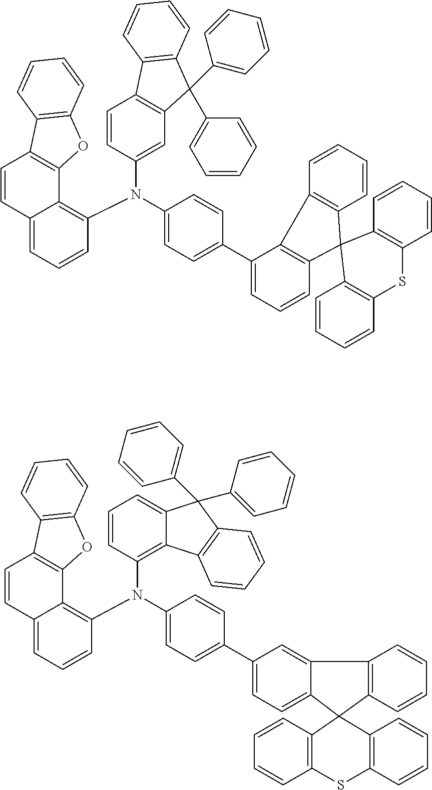 [SMILES: c1ccc(C2(c3ccccc3)c3ccccc3-c3ccc(N(c4ccc(-c5cccc6c5-c5ccccc5C65c6ccccc6Sc6ccccc65)cc4)c4cccc5ccc6c7ccccc7oc6c45)cc32)cc1 ; c1ccc(C2(c3ccccc3)c3ccccc3-c3c(N(c4ccc(-c5ccc6c(c5)-c5ccccc5C65c6ccccc6Sc6ccccc65)cc4)c4cccc5ccc6c7ccccc7oc6c45)cccc32)cc1]

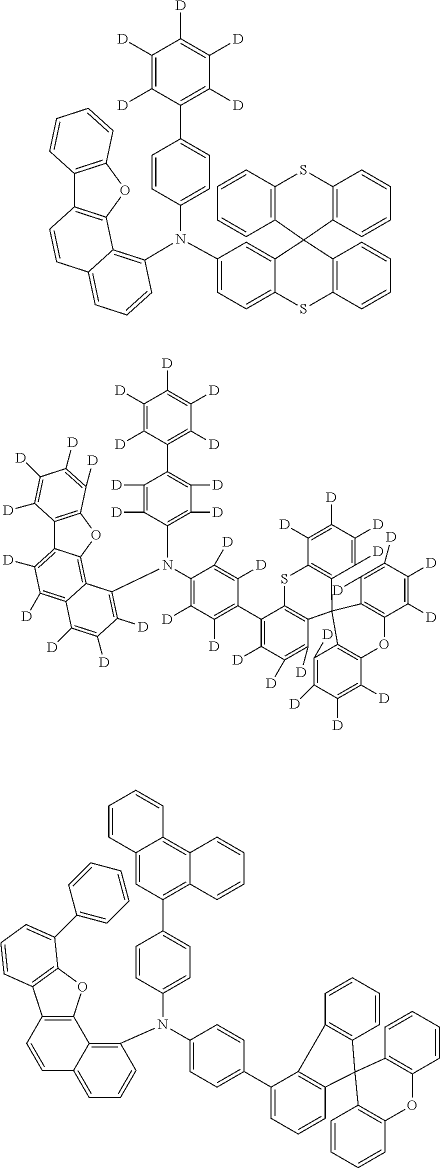

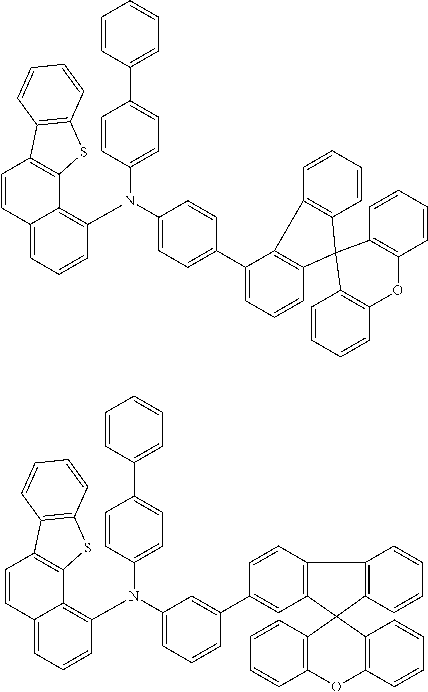 [SMILES: c1ccc(-c2ccc(N(c3ccc(-c4cccc5c4-c4ccccc4C54c5ccccc5Oc5ccccc54)cc3)c3cccc4ccc5c6ccccc6sc5c34)cc2)cc1 ; c1ccc(-c2ccc(N(c3cccc(-c4ccc5c(c4)C4(c6ccccc6Oc6ccccc64)c4ccccc4-5)c3)c3cccc4ccc5c6ccccc6sc5c34)cc2)cc1]

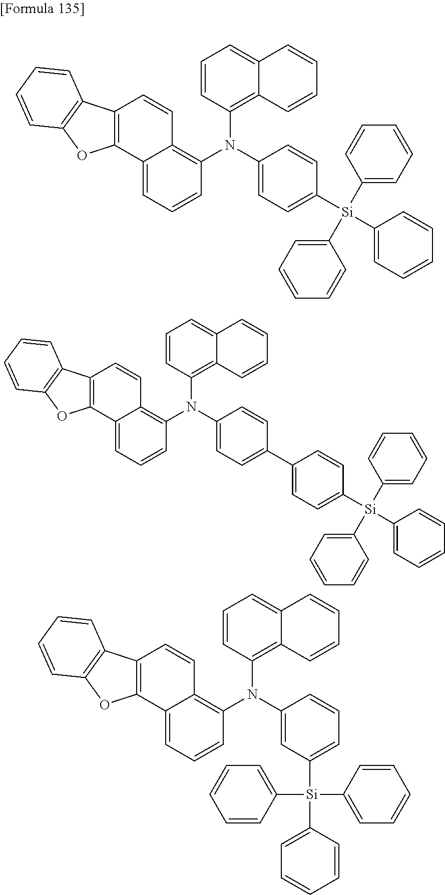

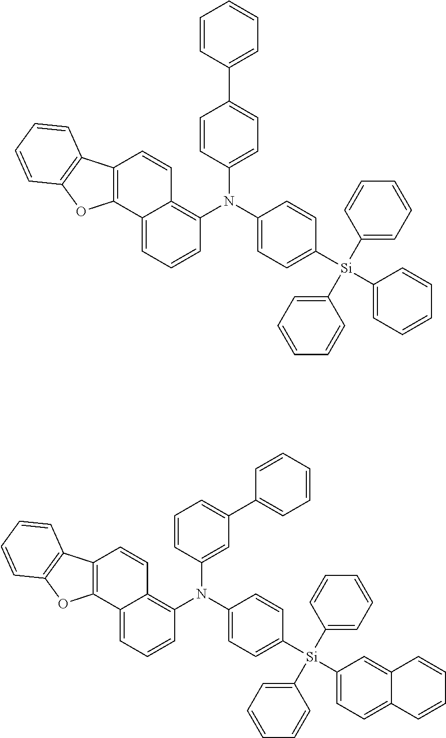 [SMILES: c1ccc(-c2ccc(N(c3ccc([Si](c4ccccc4)(c4ccccc4)c4ccccc4)cc3)c3cccc4c3ccc3c5ccccc5oc43)cc2)cc1 ; c1ccc(-c2cccc(N(c3ccc([Si](c4ccccc4)(c4ccccc4)c4ccc5ccccc5c4)cc3)c3cccc4c3ccc3c5ccccc5oc43)c2)cc1]

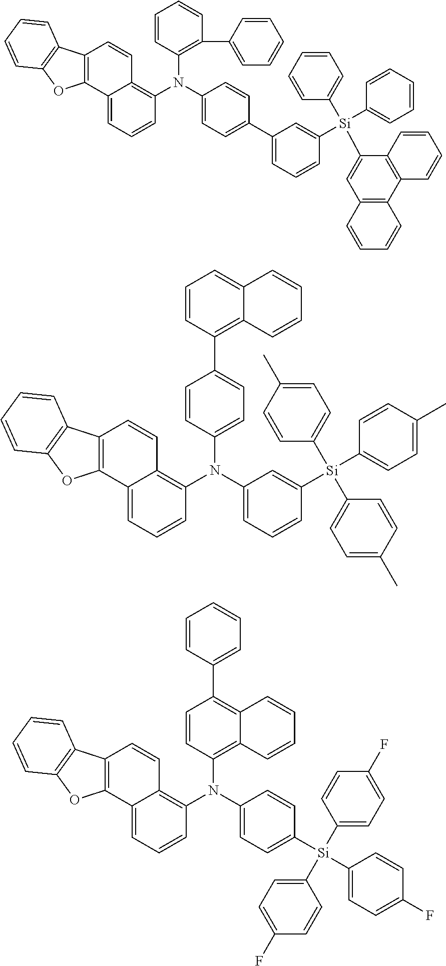 [SMILES: c1ccc(-c2ccccc2N(c2ccc(-c3cccc([Si](c4ccccc4)(c4ccccc4)c4cc5ccccc5c5ccccc45)c3)cc2)c2cccc3c2ccc2c4ccccc4oc32)cc1 ; Cc1ccc([Si](c2ccc(C)cc2)(c2ccc(C)cc2)c2cccc(N(c3ccc(-c4cccc5ccccc45)cc3)c3cccc4c3ccc3c5ccccc5oc43)c2)cc1 ; Fc1ccc([Si](c2ccc(F)cc2)(c2ccc(F)cc2)c2ccc(N(c3ccc(-c4ccccc4)c4ccccc34)c3cccc4c3ccc3c5ccccc5oc43)cc2)cc1]

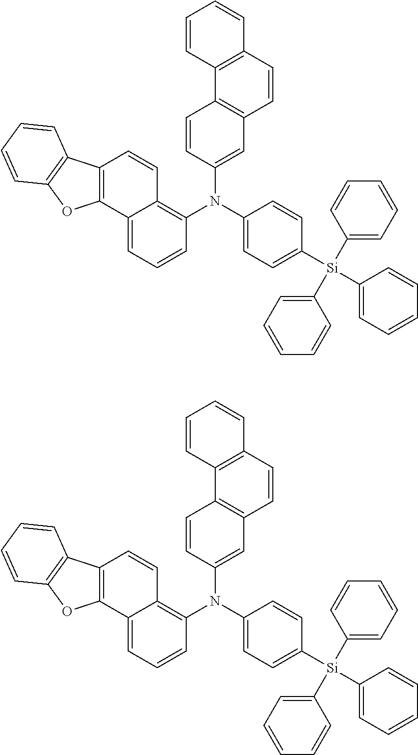 [SMILES: c1ccc([Si](c2ccccc2)(c2ccccc2)c2ccc(N(c3ccc4c(ccc5ccccc54)c3)c3cccc4c3ccc3c5ccccc5oc43)cc2)cc1 ; c1ccc([Si](c2ccccc2)(c2ccccc2)c2ccc(N(c3ccc4c(ccc5ccccc54)c3)c3cccc4c3ccc3c5ccccc5oc43)cc2)cc1]

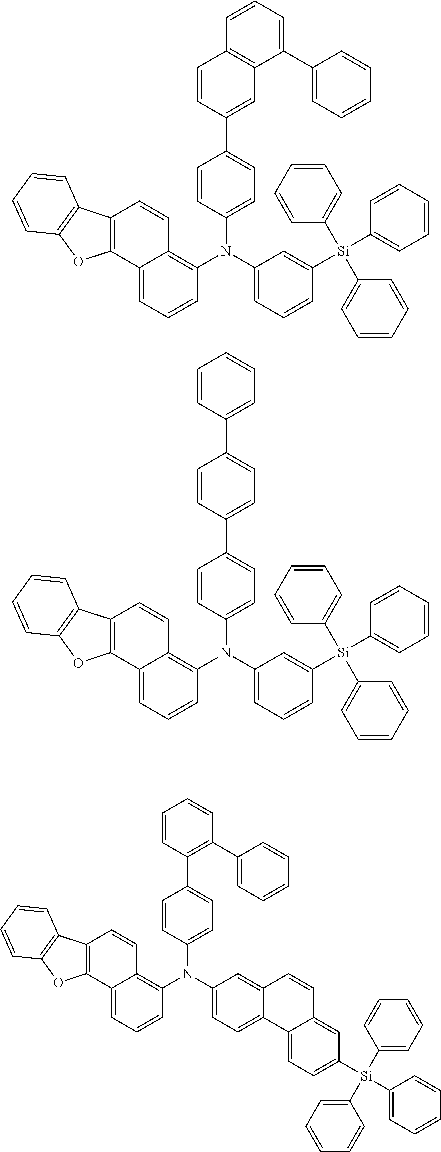 [SMILES: c1ccc(-c2cccc3ccc(-c4ccc(N(c5cccc([Si](c6ccccc6)(c6ccccc6)c6ccccc6)c5)c5cccc6c5ccc5c7ccccc7oc65)cc4)cc23)cc1 ; c1ccc(-c2ccc(-c3ccc(N(c4cccc([Si](c5ccccc5)(c5ccccc5)c5ccccc5)c4)c4cccc5c4ccc4c6ccccc6oc54)cc3)cc2)cc1 ; c1ccc(-c2ccccc2-c2ccc(N(c3ccc4c(ccc5cc([Si](c6ccccc6)(c6ccccc6)c6ccccc6)ccc54)c3)c3cccc4c3ccc3c5ccccc5oc43)cc2)cc1]

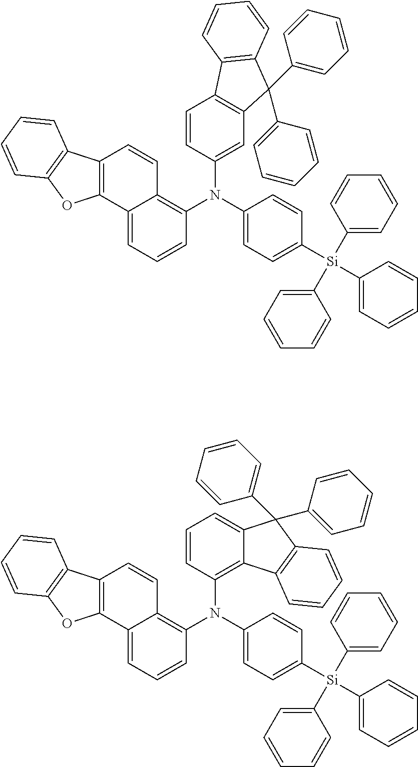 [SMILES: c1ccc(C2(c3ccccc3)c3ccccc3-c3ccc(N(c4ccc([Si](c5ccccc5)(c5ccccc5)c5ccccc5)cc4)c4cccc5c4ccc4c6ccccc6oc54)cc32)cc1 ; c1ccc(C2(c3ccccc3)c3ccccc3-c3c(N(c4ccc([Si](c5ccccc5)(c5ccccc5)c5ccccc5)cc4)c4cccc5c4ccc4c6ccccc6oc54)cccc32)cc1]

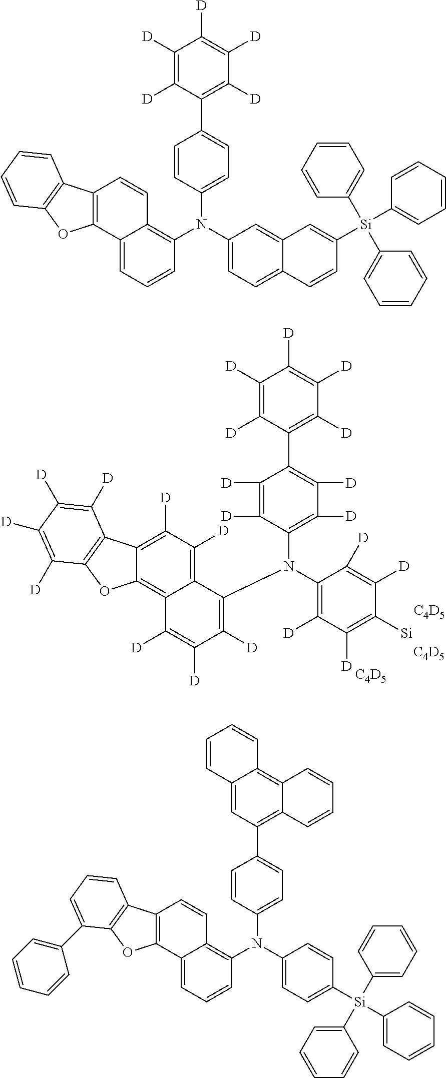

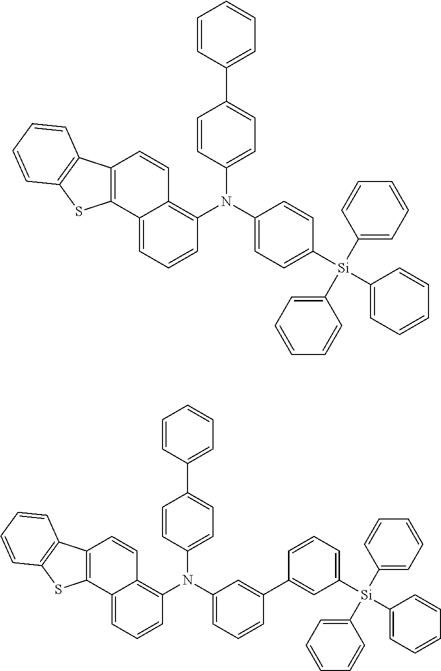 [SMILES: c1ccc(-c2ccc(N(c3ccc([Si](c4ccccc4)(c4ccccc4)c4ccccc4)cc3)c3cccc4c3ccc3c5ccccc5sc43)cc2)cc1 ; c1ccc(-c2ccc(N(c3cccc(-c4cccc([Si](c5ccccc5)(c5ccccc5)c5ccccc5)c4)c3)c3cccc4c3ccc3c5ccccc5sc43)cc2)cc1]

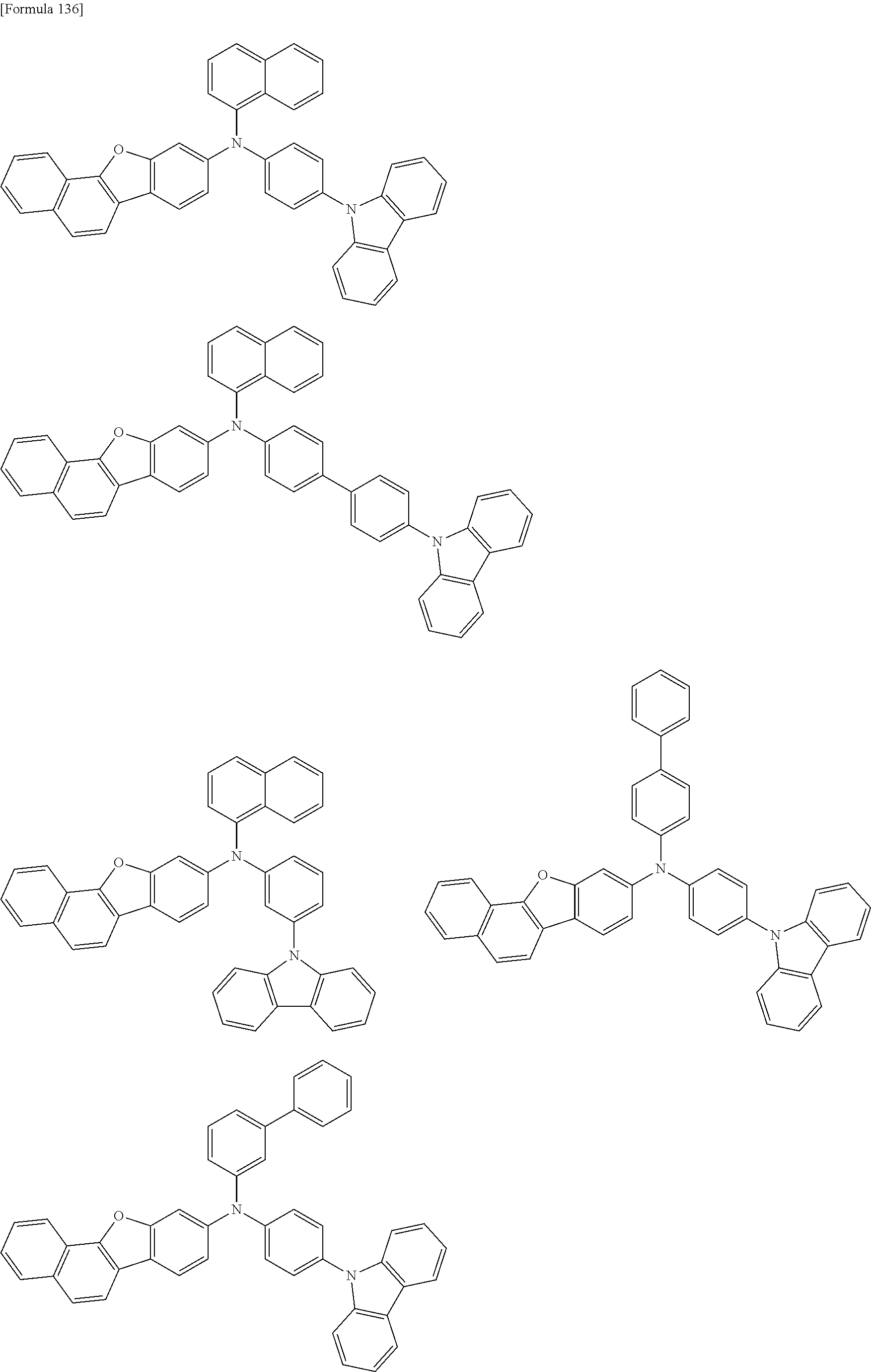

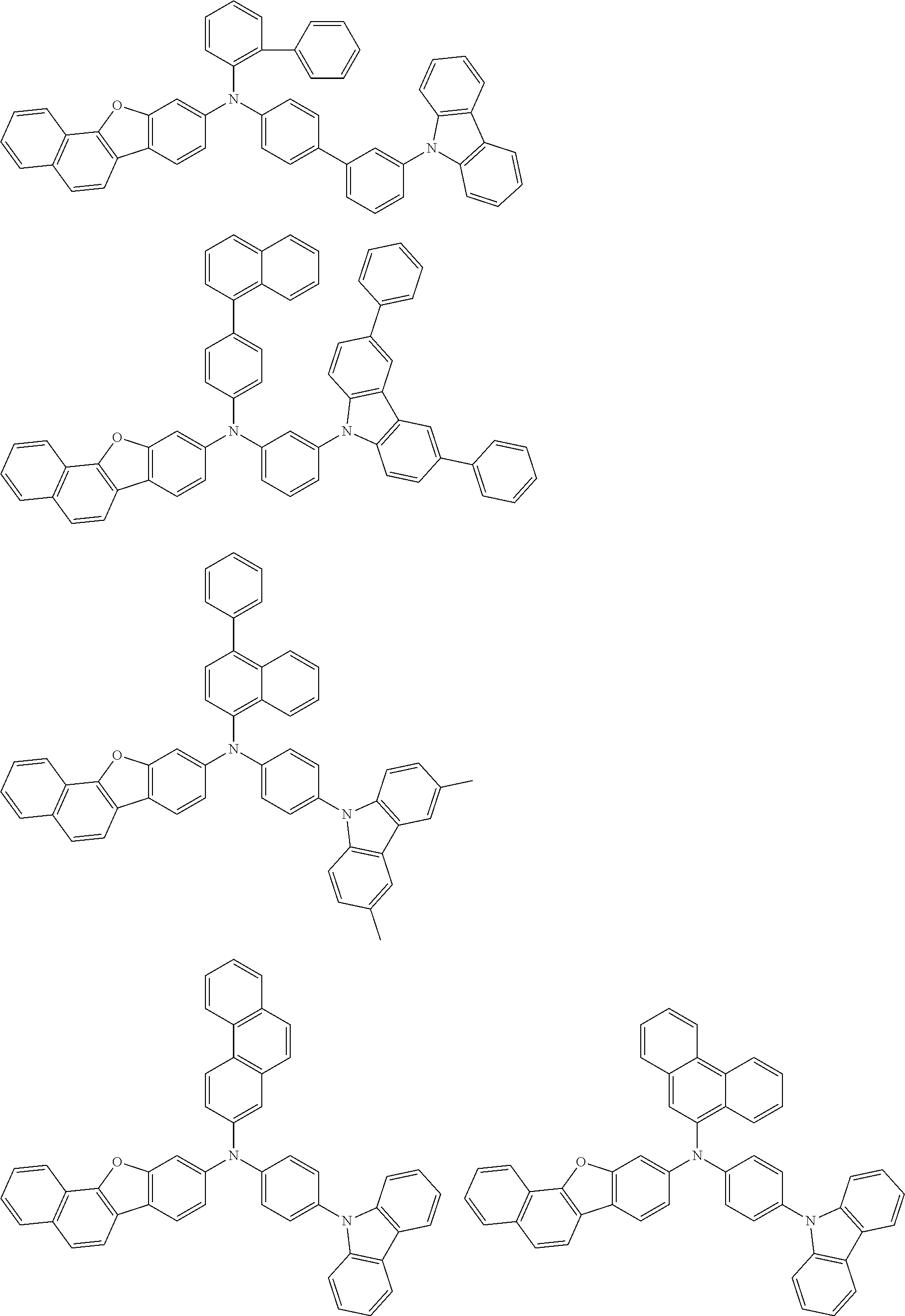

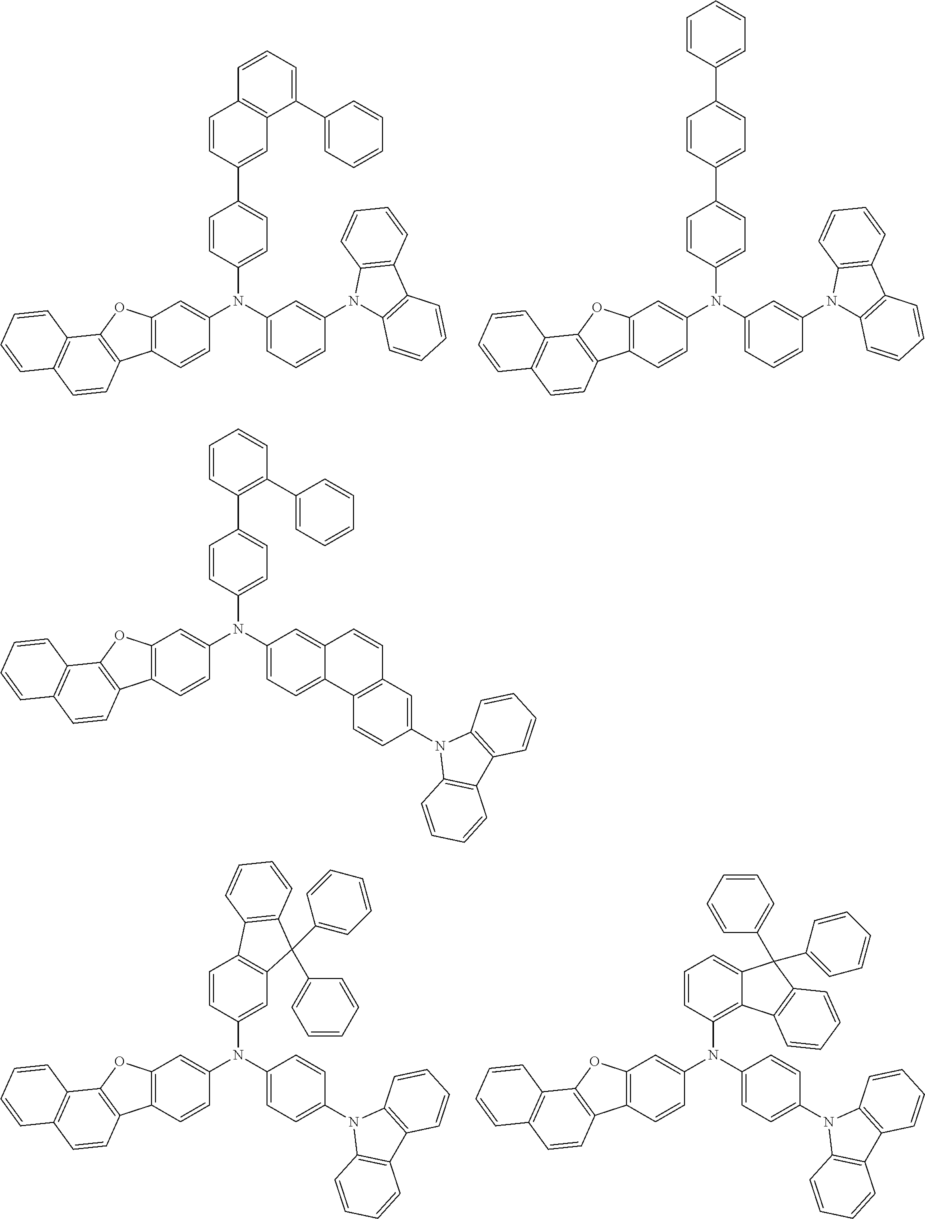

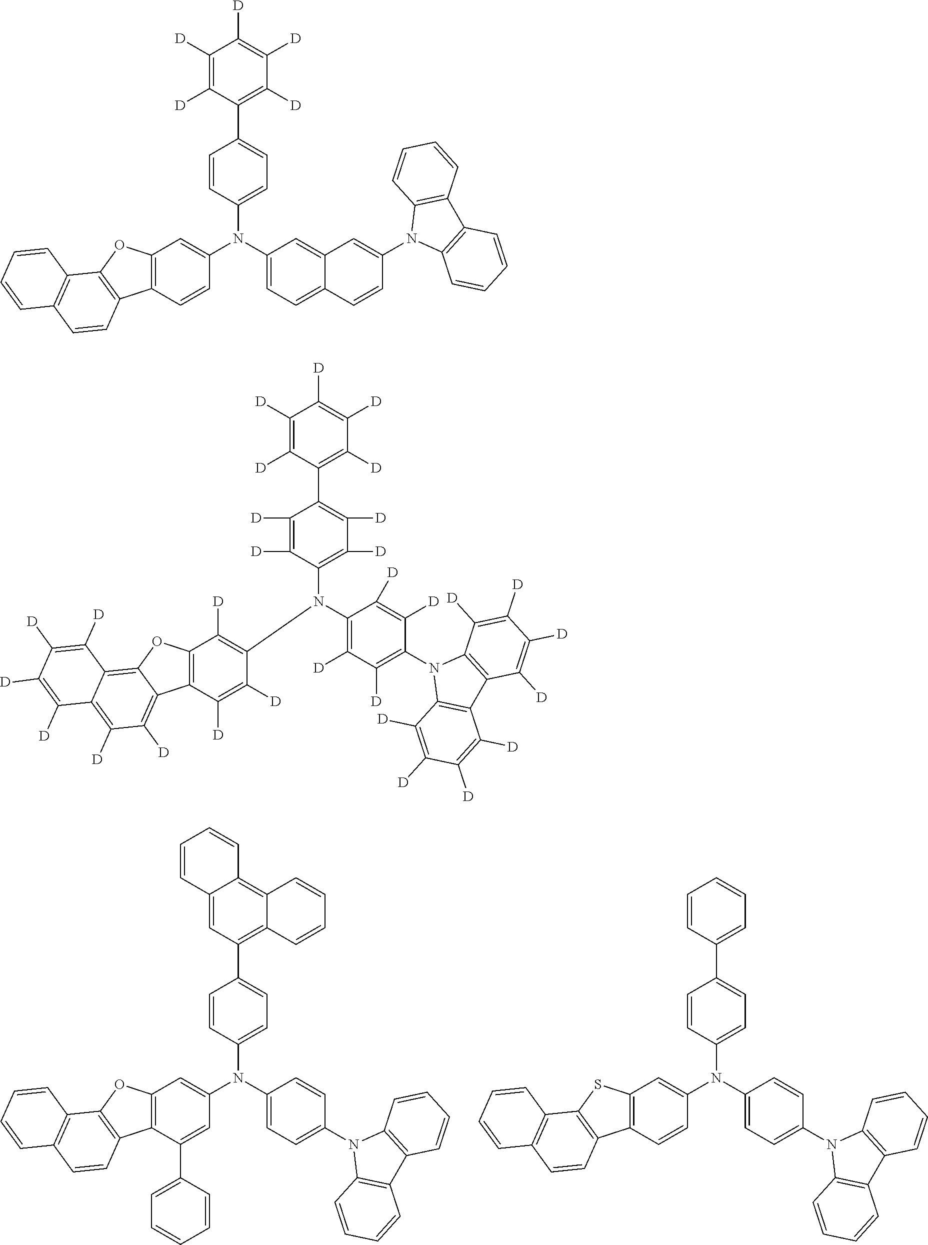

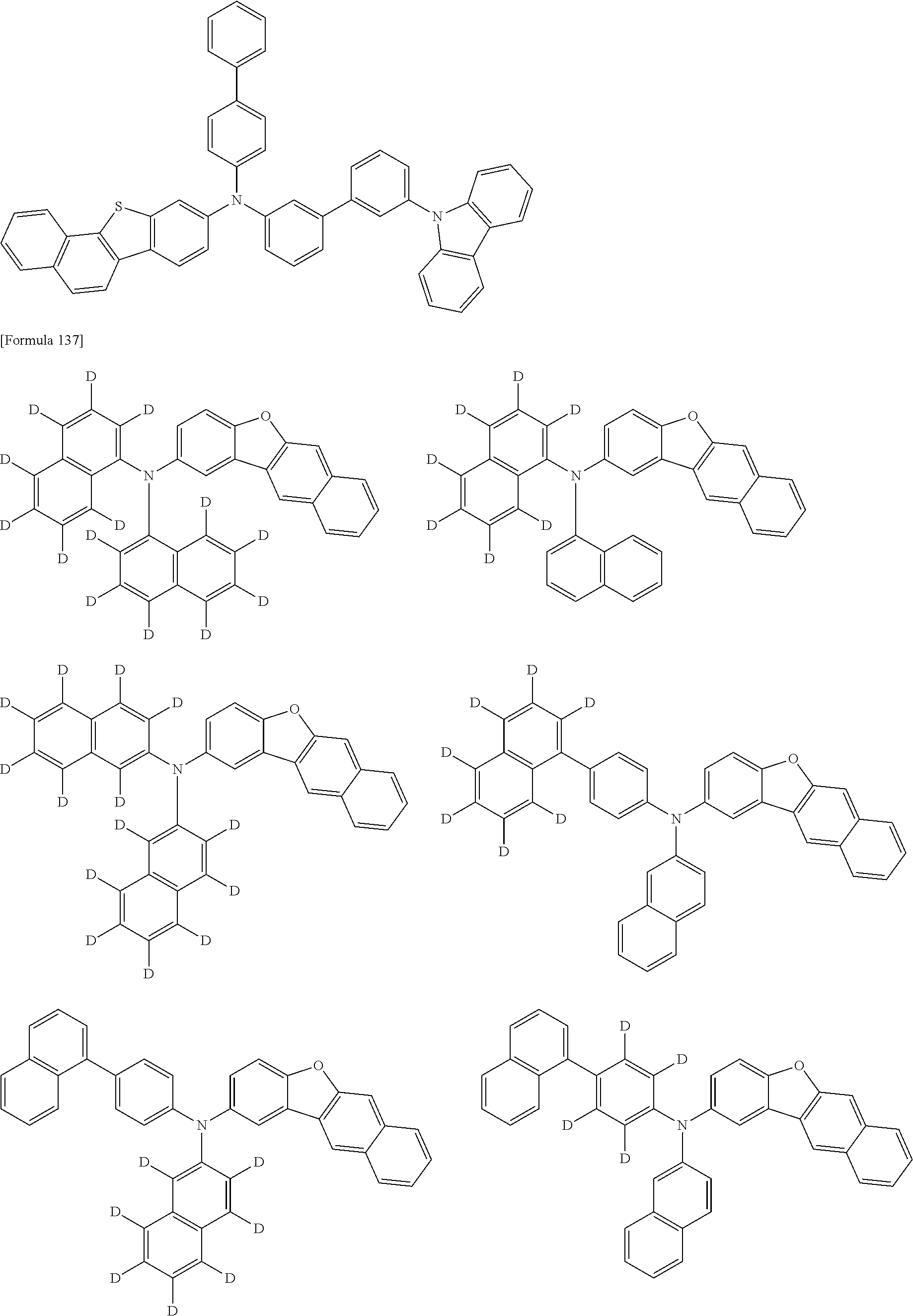

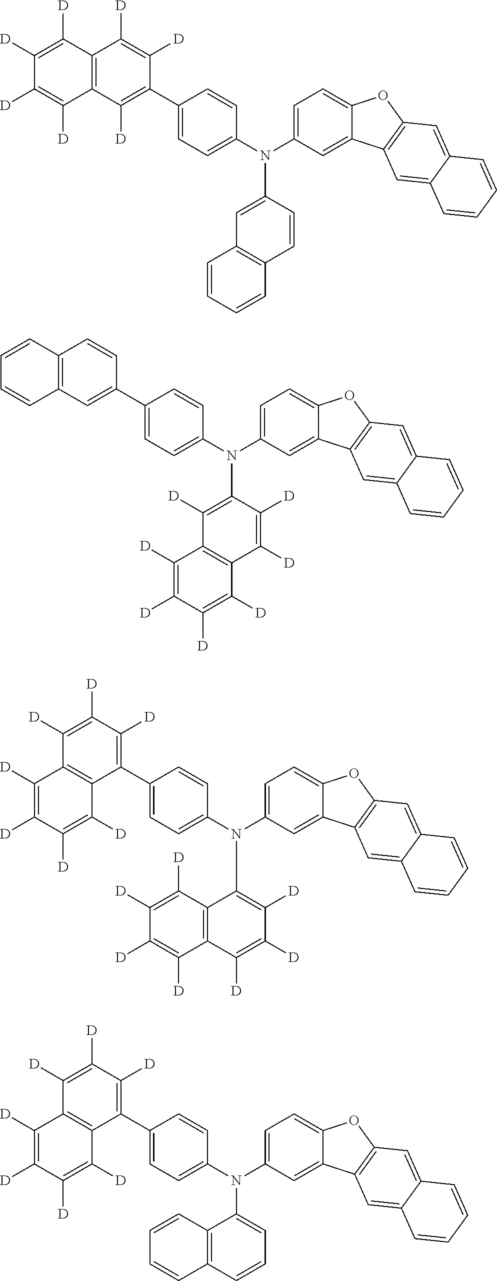 [SMILES: [2H]c1c([2H])c([2H])c2c([2H])c(-c3ccc(N(c4ccc5ccccc5c4)c4ccc5oc6cc7ccccc7cc6c5c4)cc3)c([2H])c([2H])c2c1[2H] ; [2H]c1c([2H])c([2H])c2c([2H])c(N(c3ccc(-c4ccc5ccccc5c4)cc3)c3ccc4oc5cc6ccccc6cc5c4c3)c([2H])c([2H])c2c1[2H] ; [2H]c1c([2H])c([2H])c2c(-c3ccc(N(c4ccc5oc6cc7ccccc7cc6c5c4)c4c([2H])c([2H])c([2H])c5c([2H])c([2H])c([2H])c([2H])c45)cc3)c([2H])c([2H])c([2H])c2c1[2H] ; [2H]c1c([2H])c([2H])c2c(-c3ccc(N(c4ccc5oc6cc7ccccc7cc6c5c4)c4cccc5ccccc45)cc3)c([2H])c([2H])c([2H])c2c1[2H]]

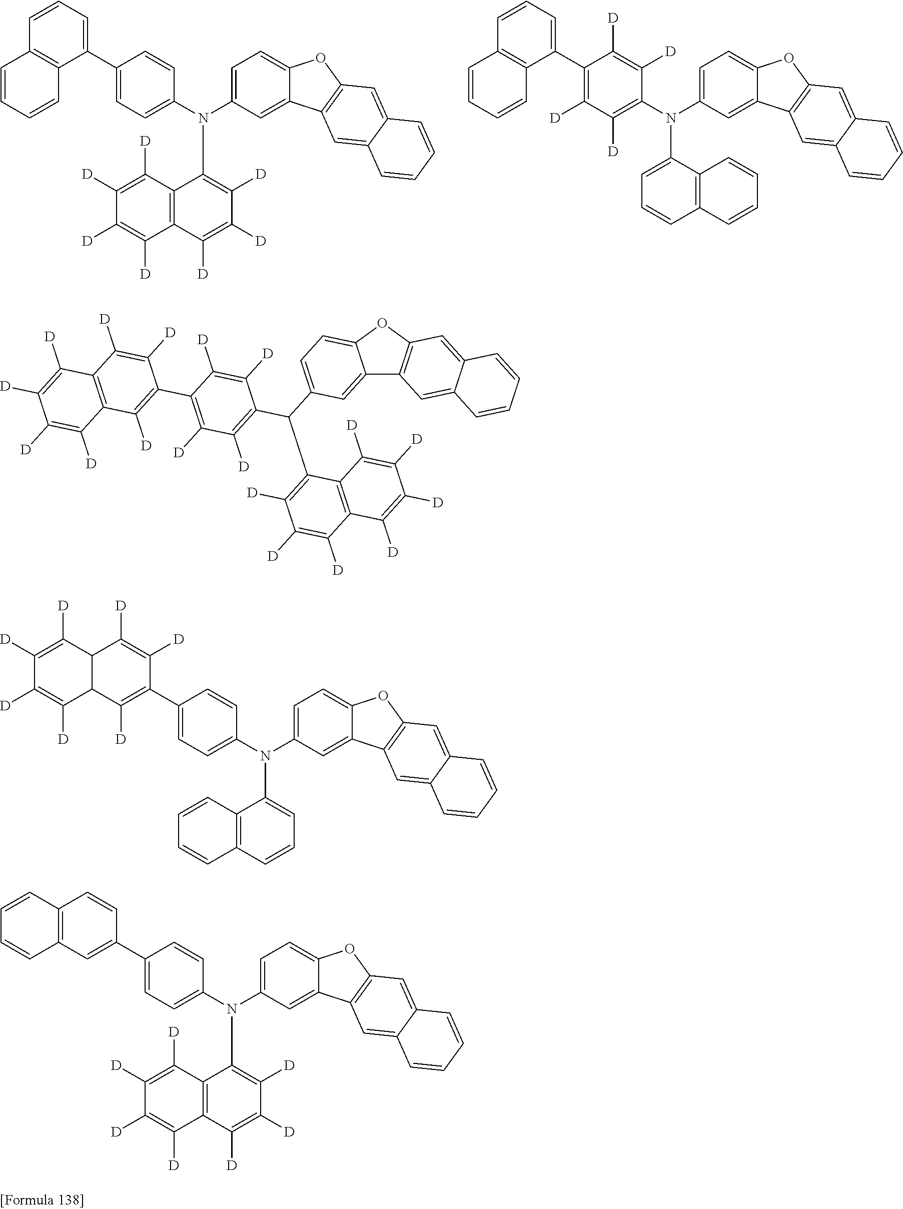

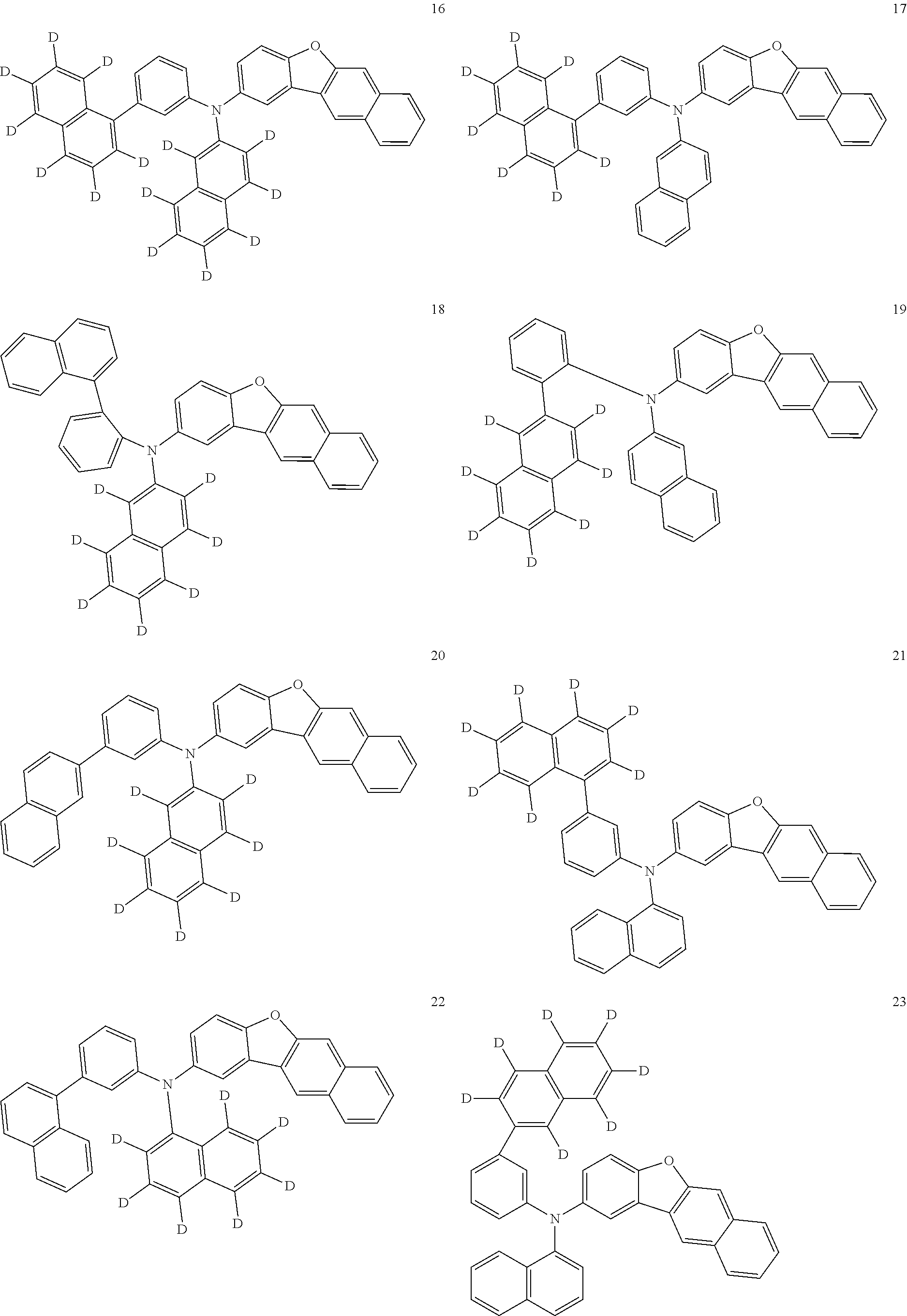

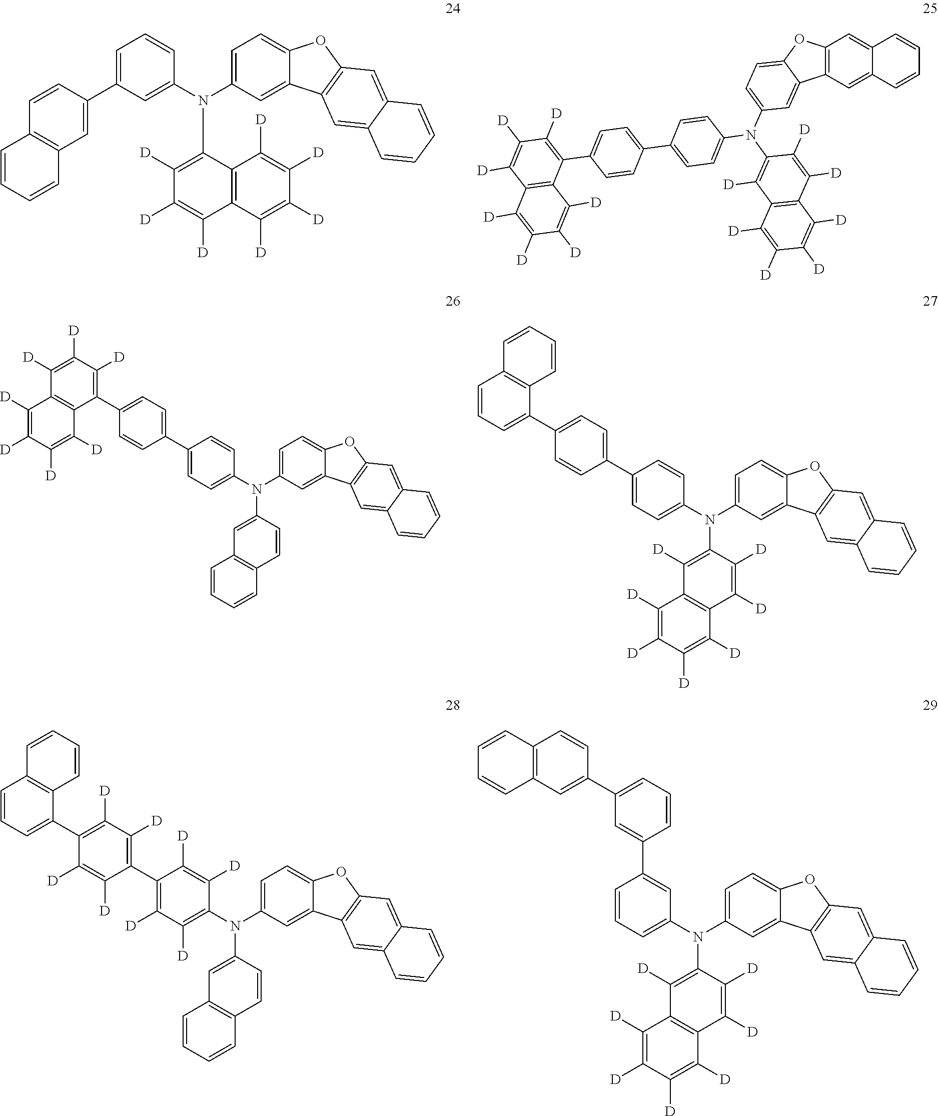

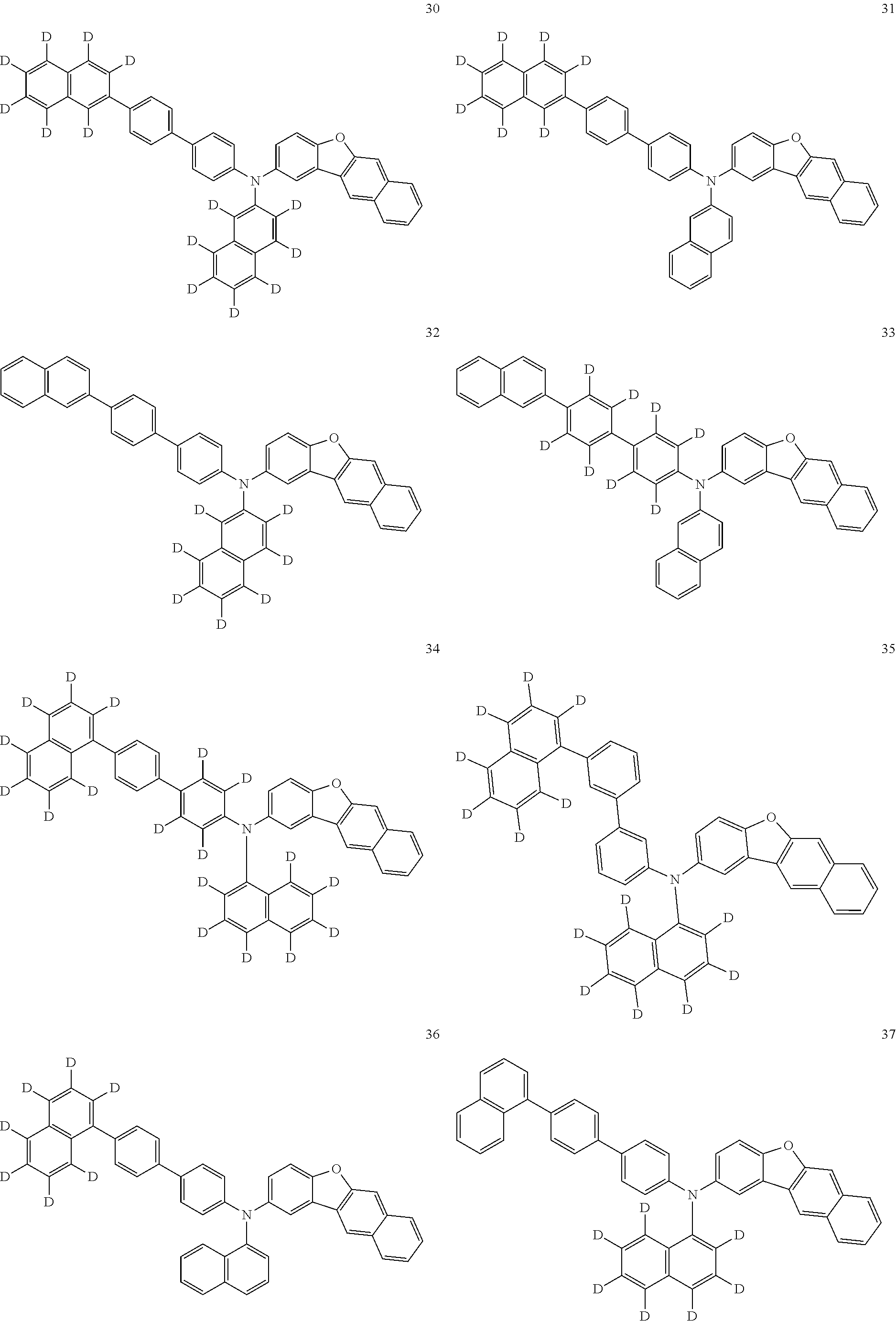

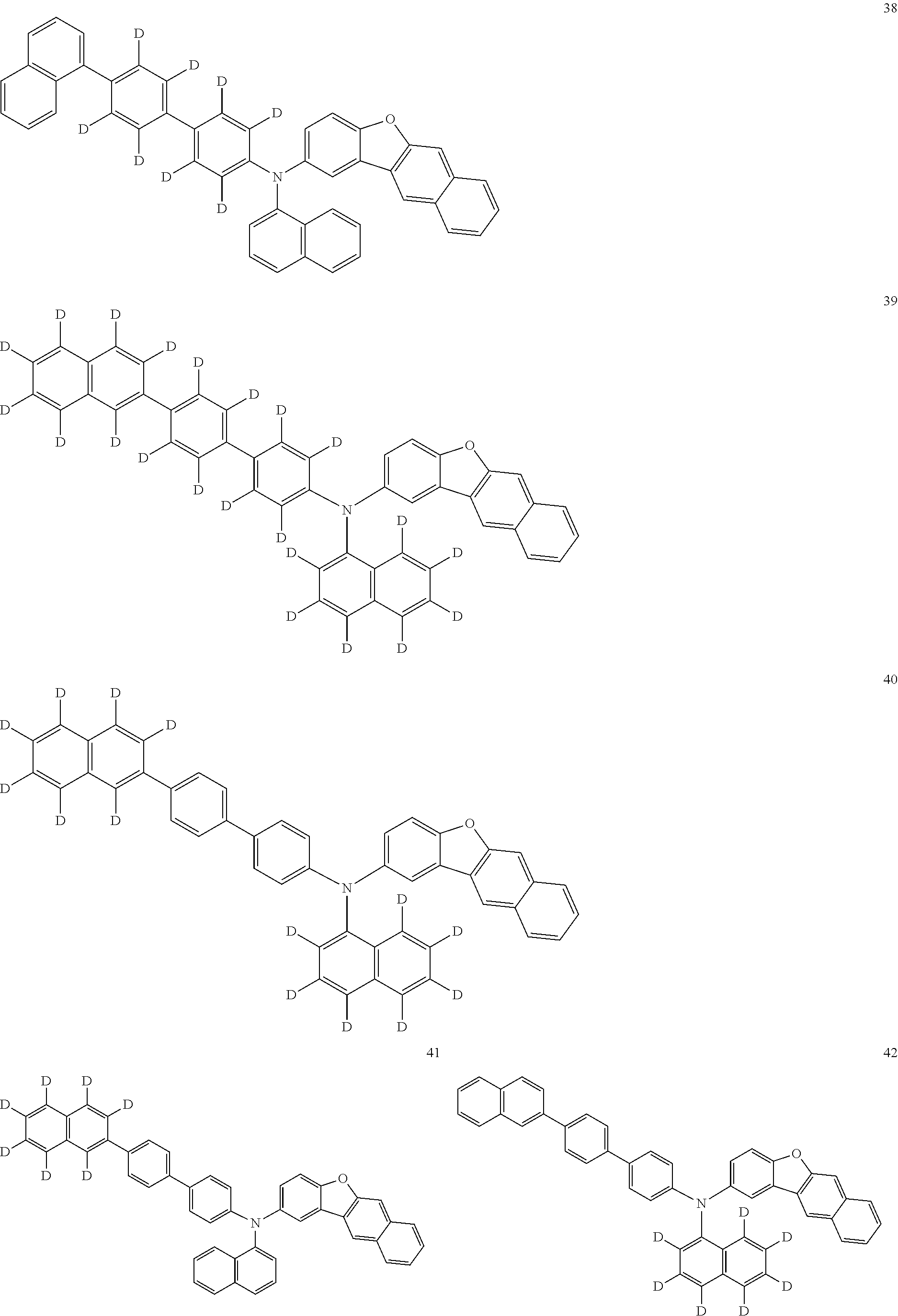

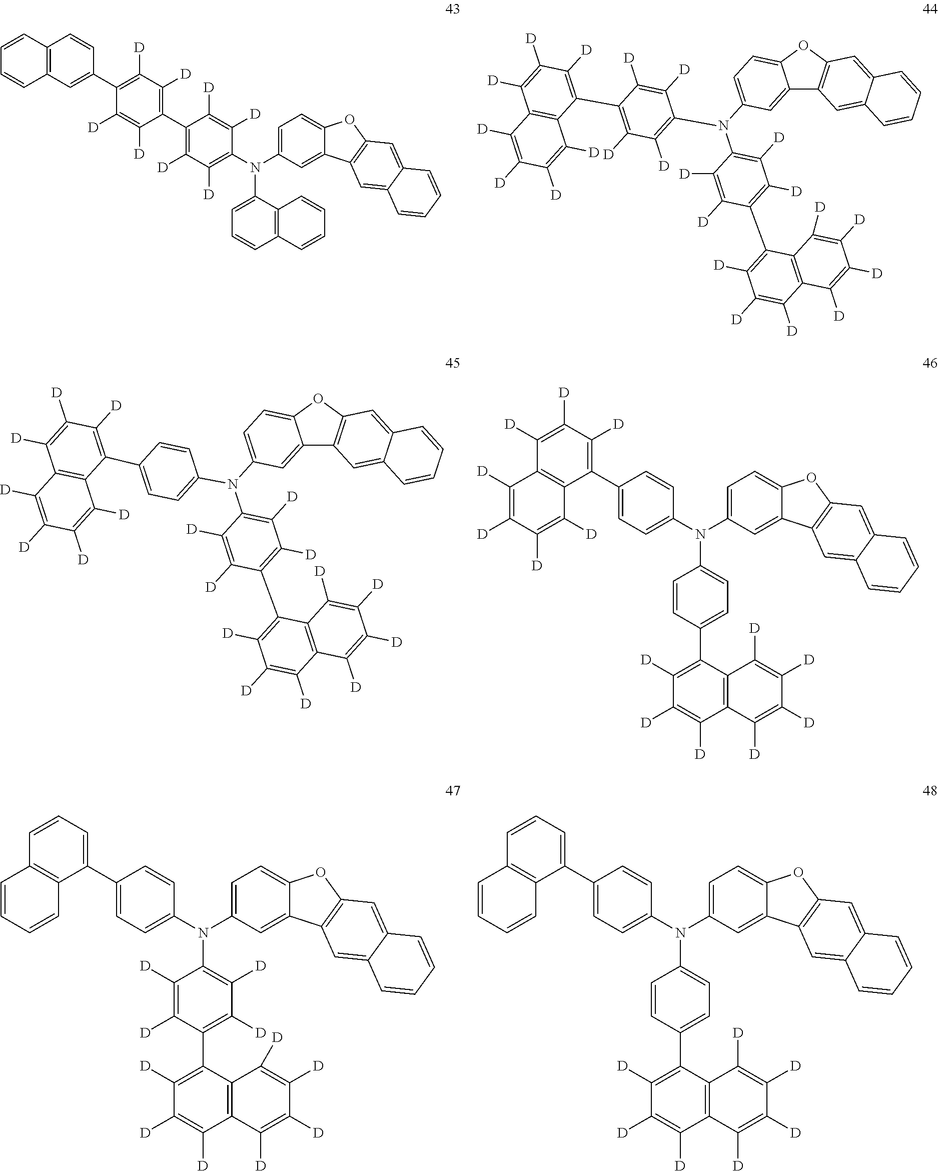

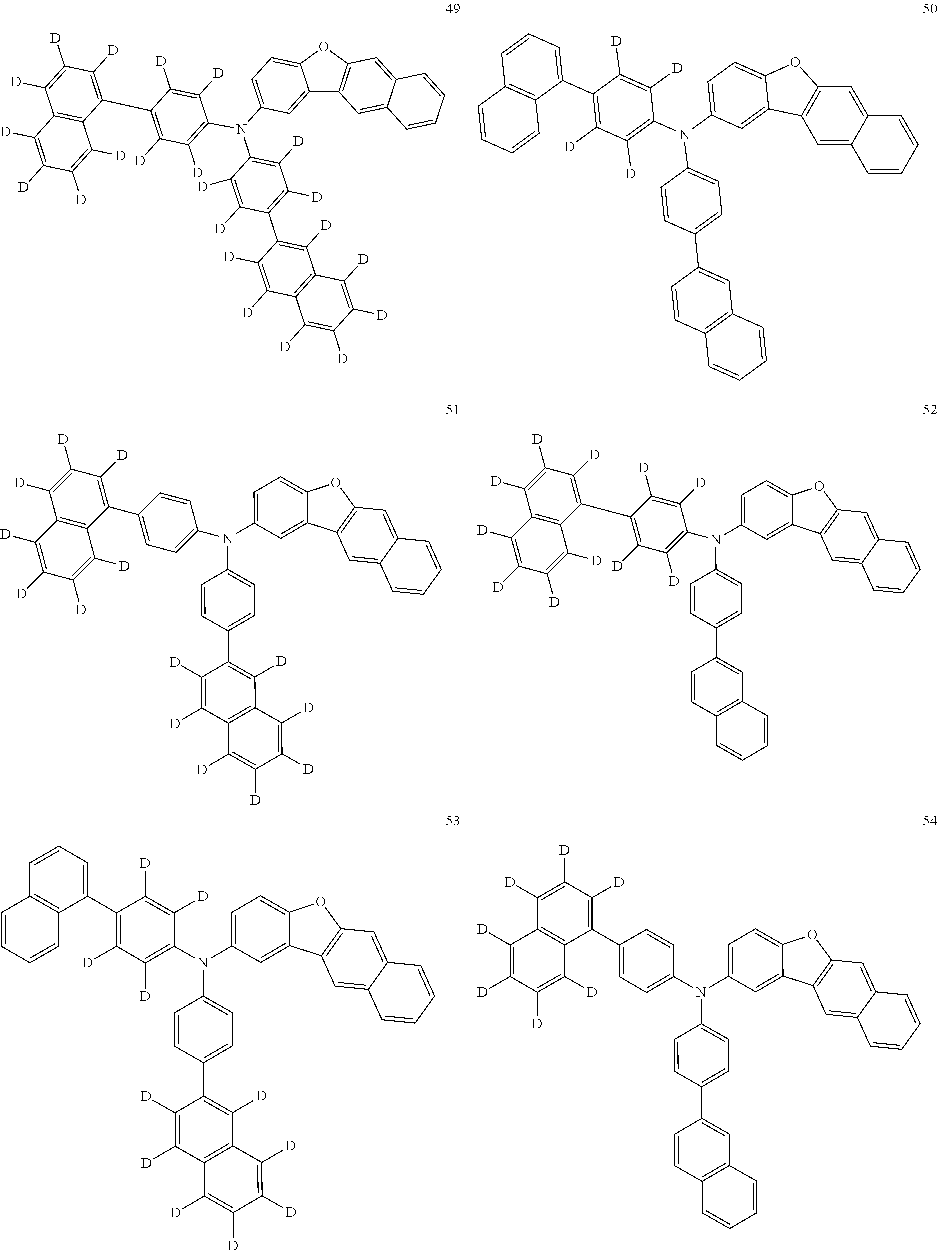

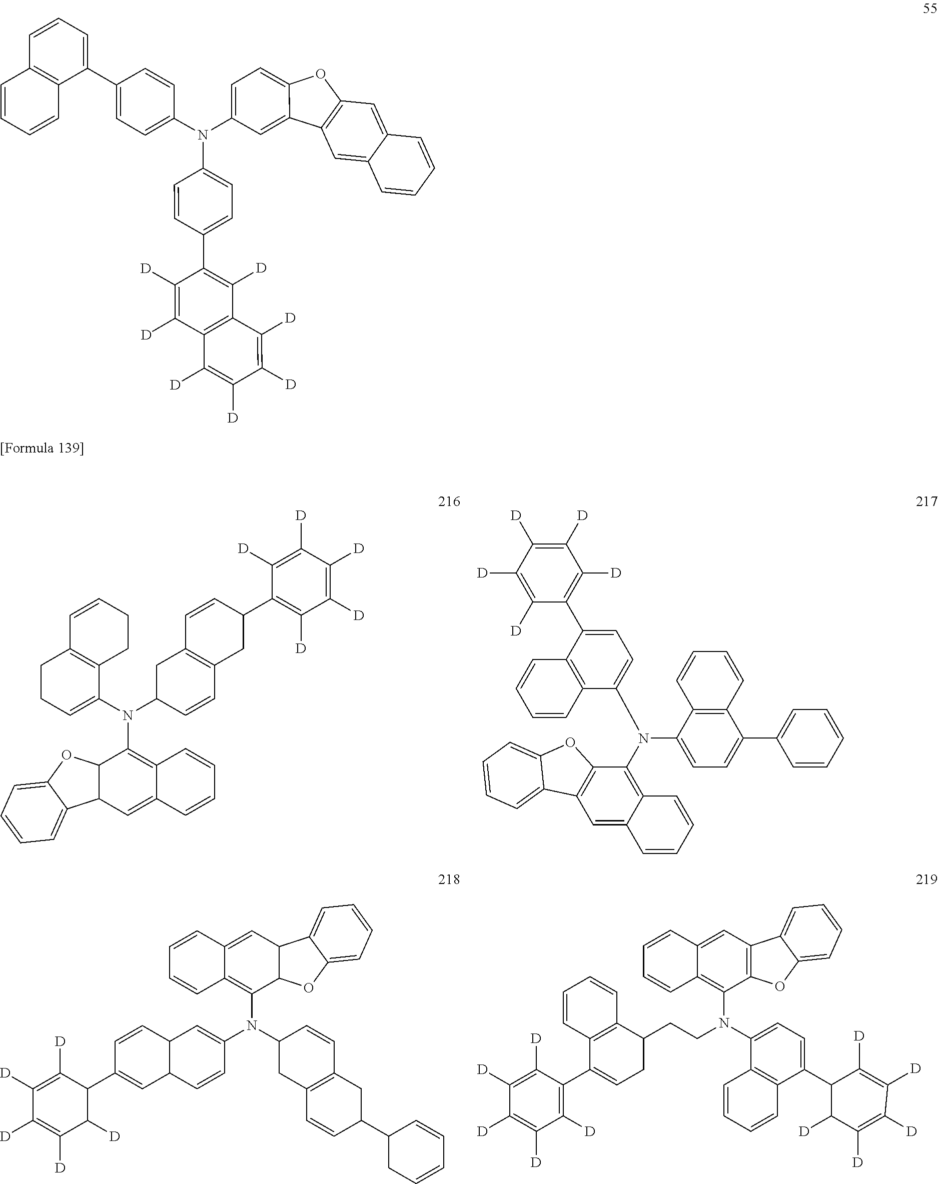

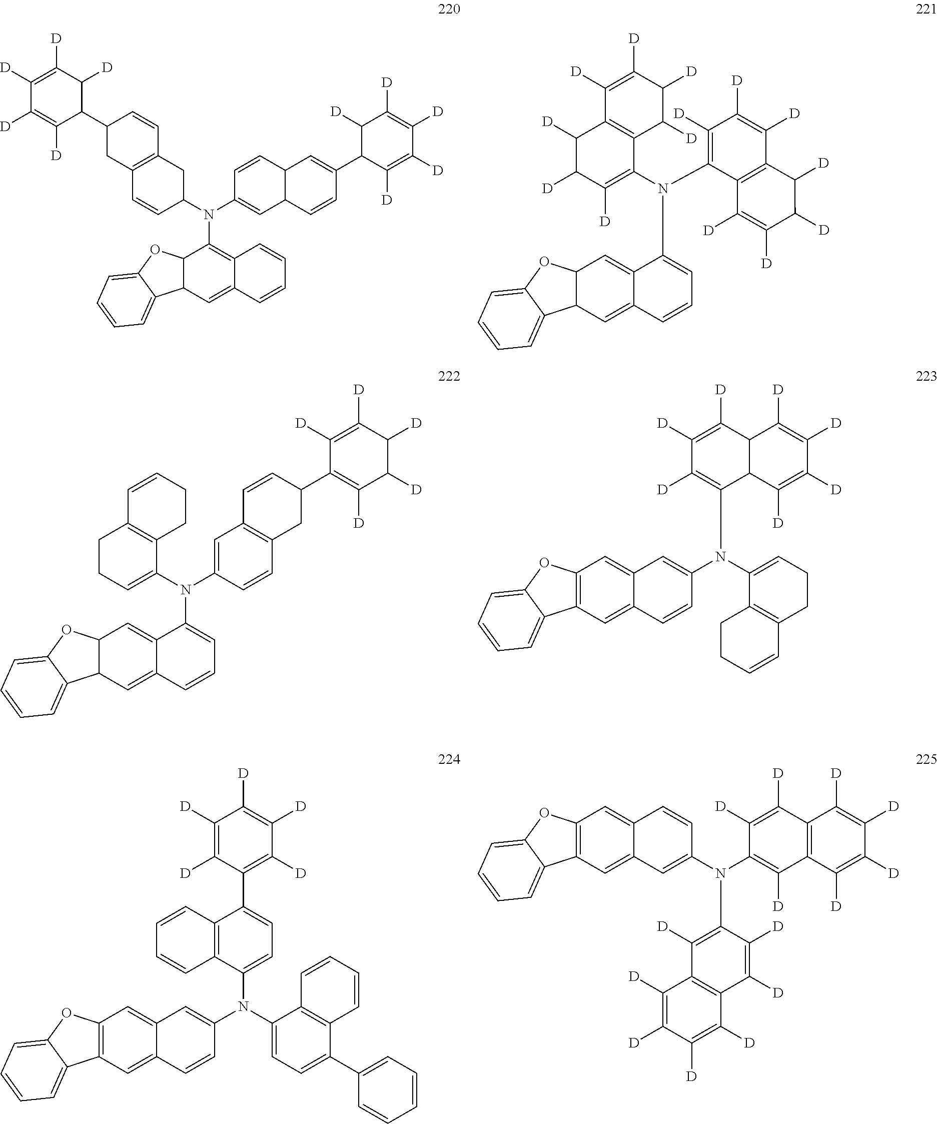

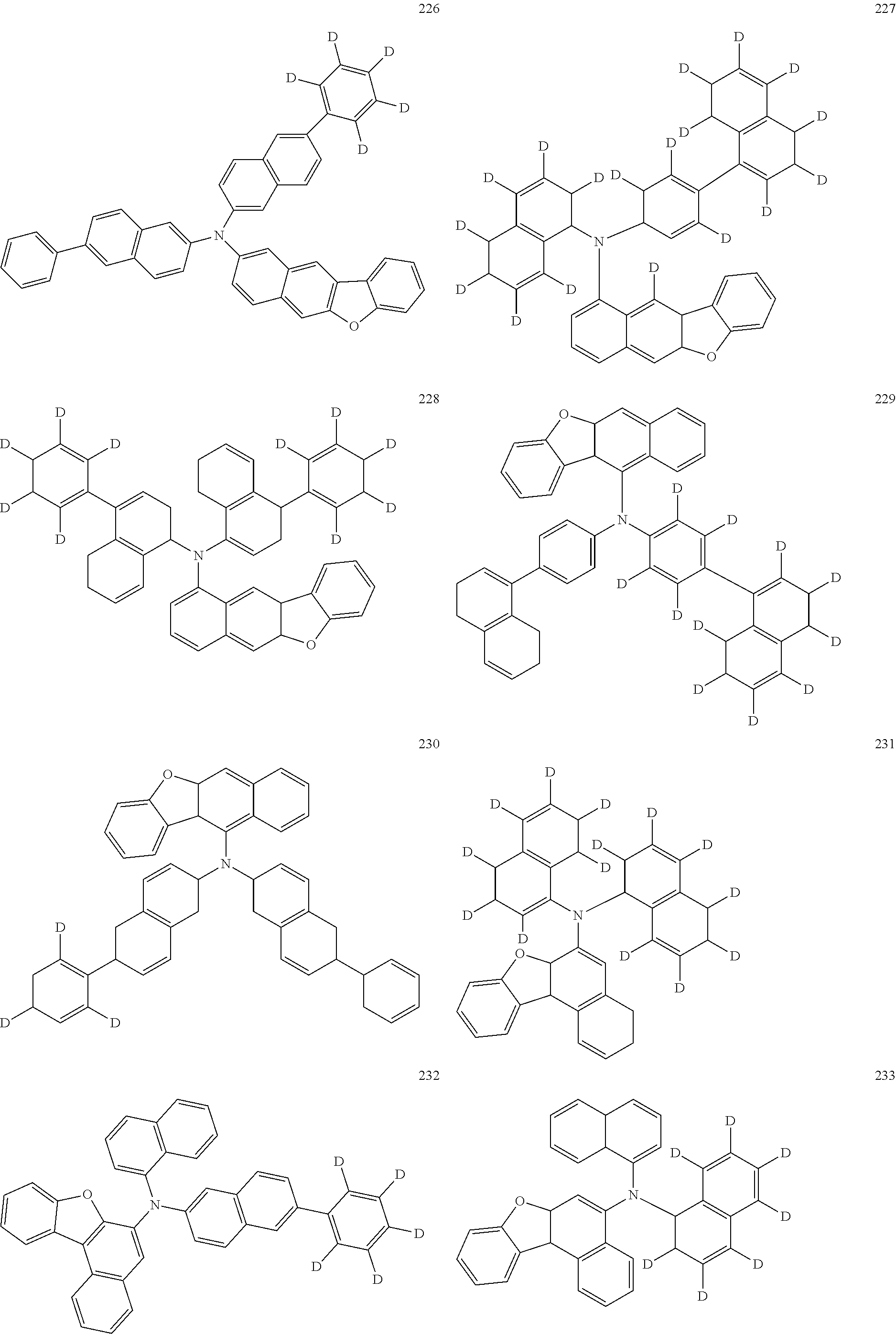

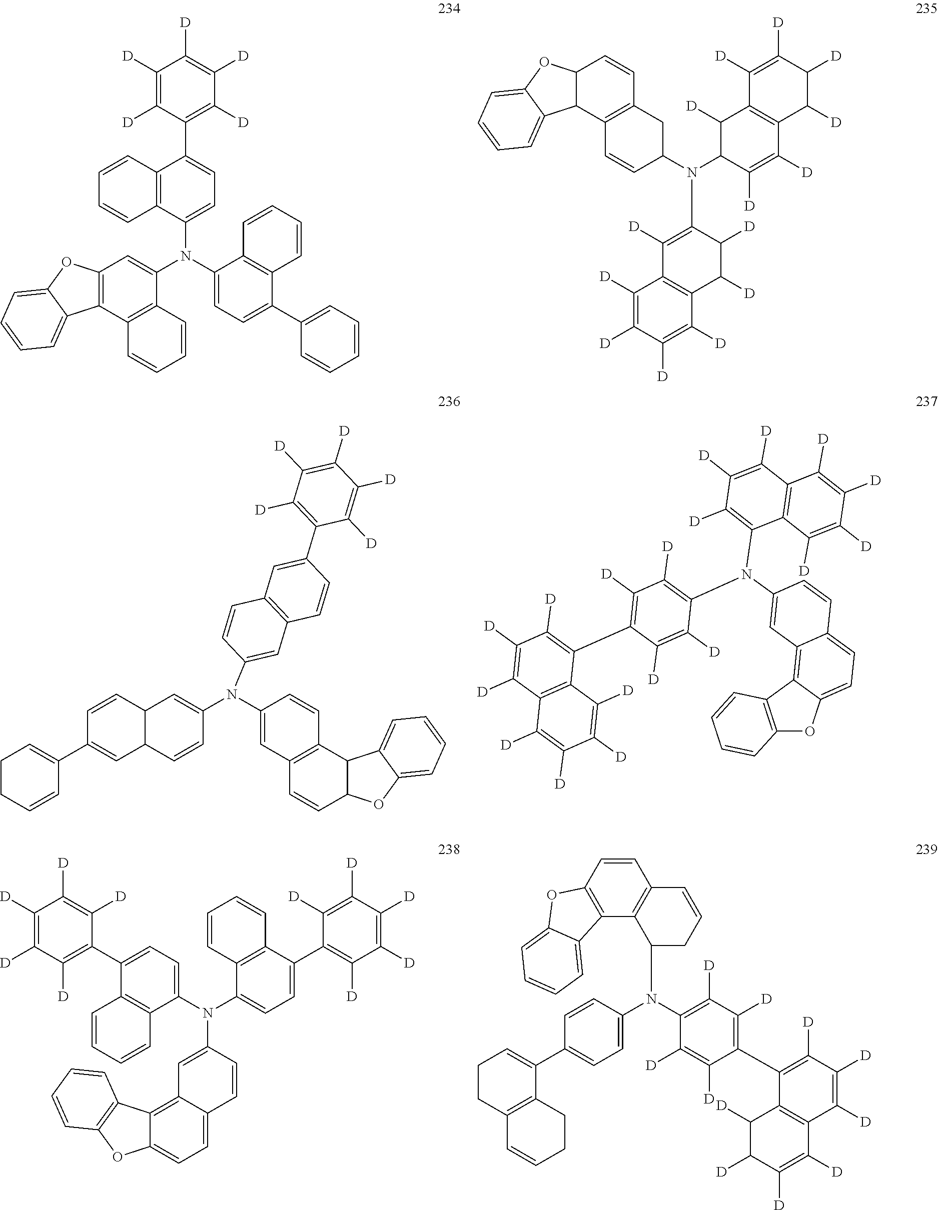

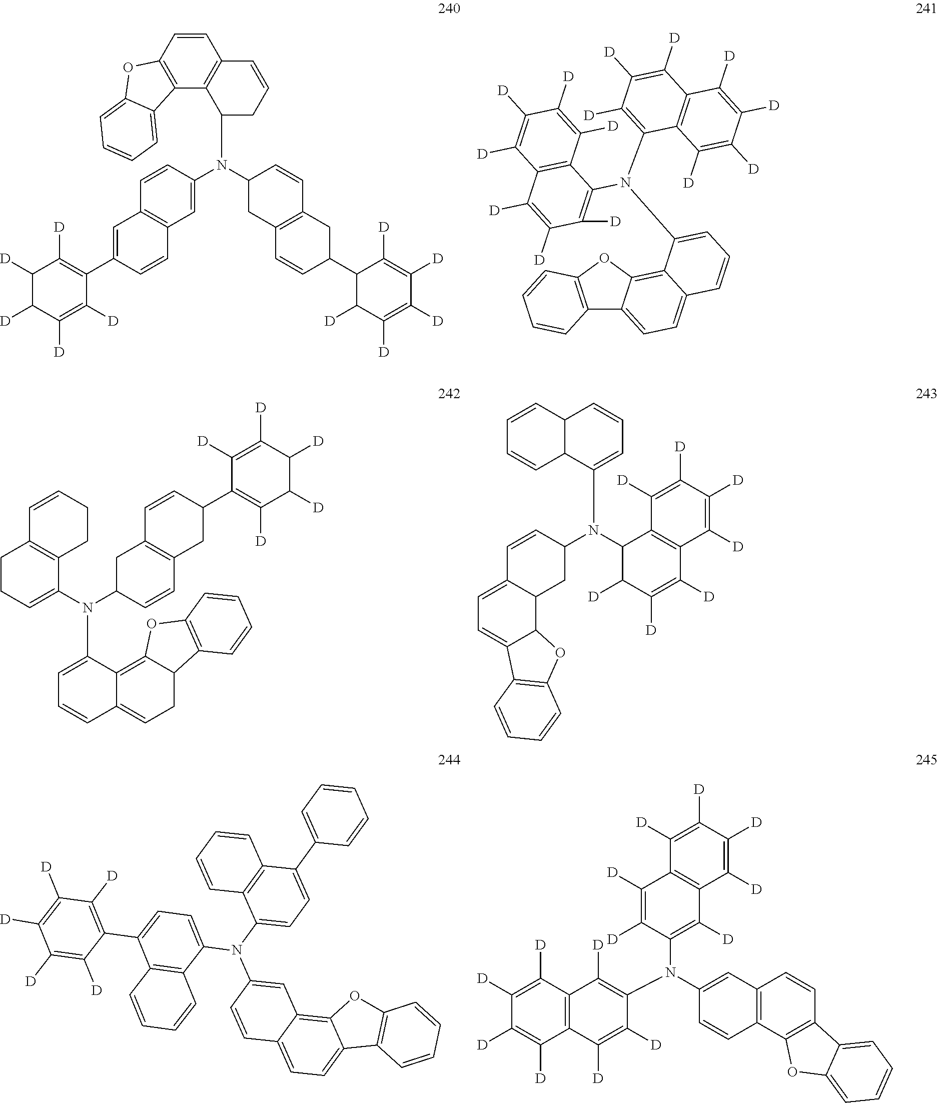

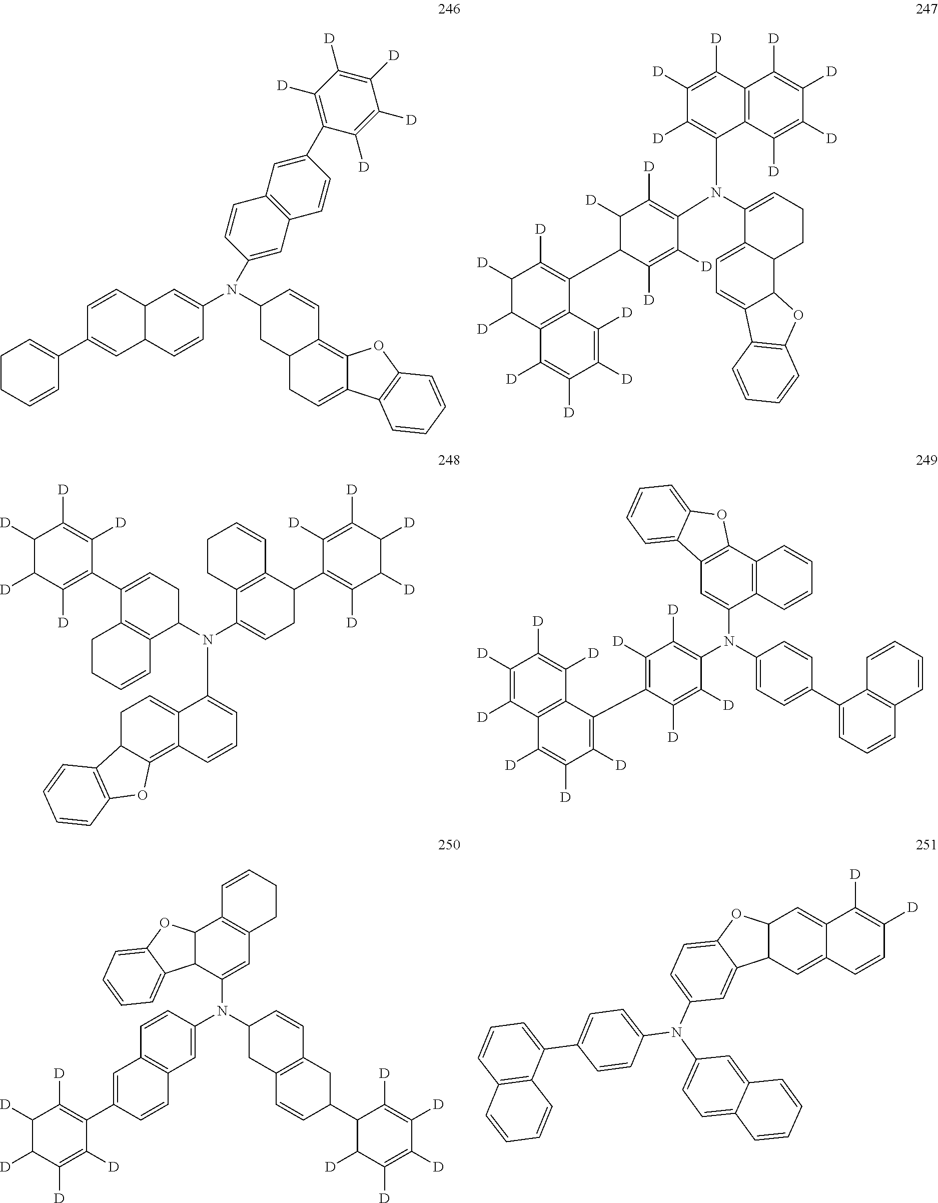

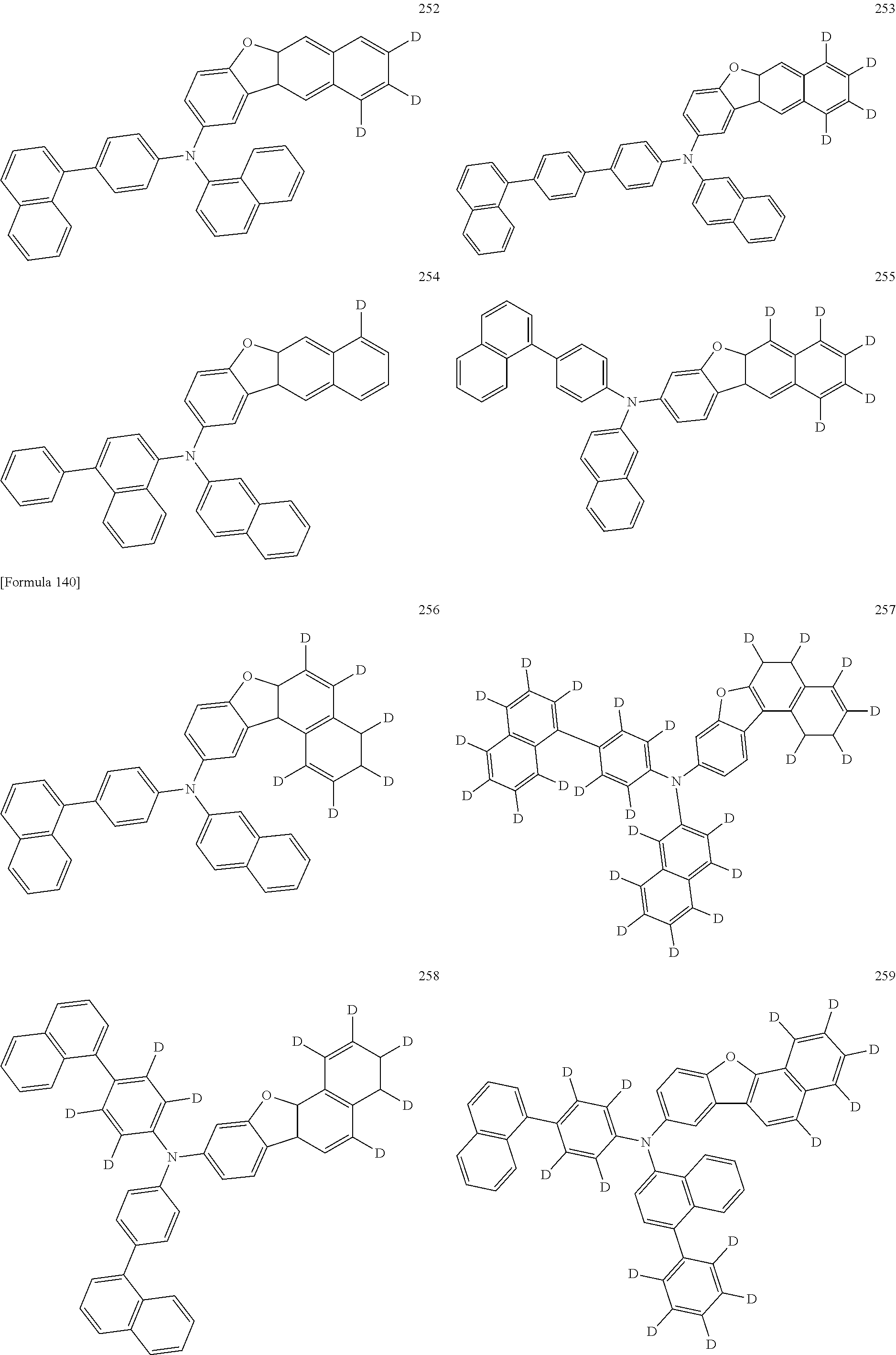

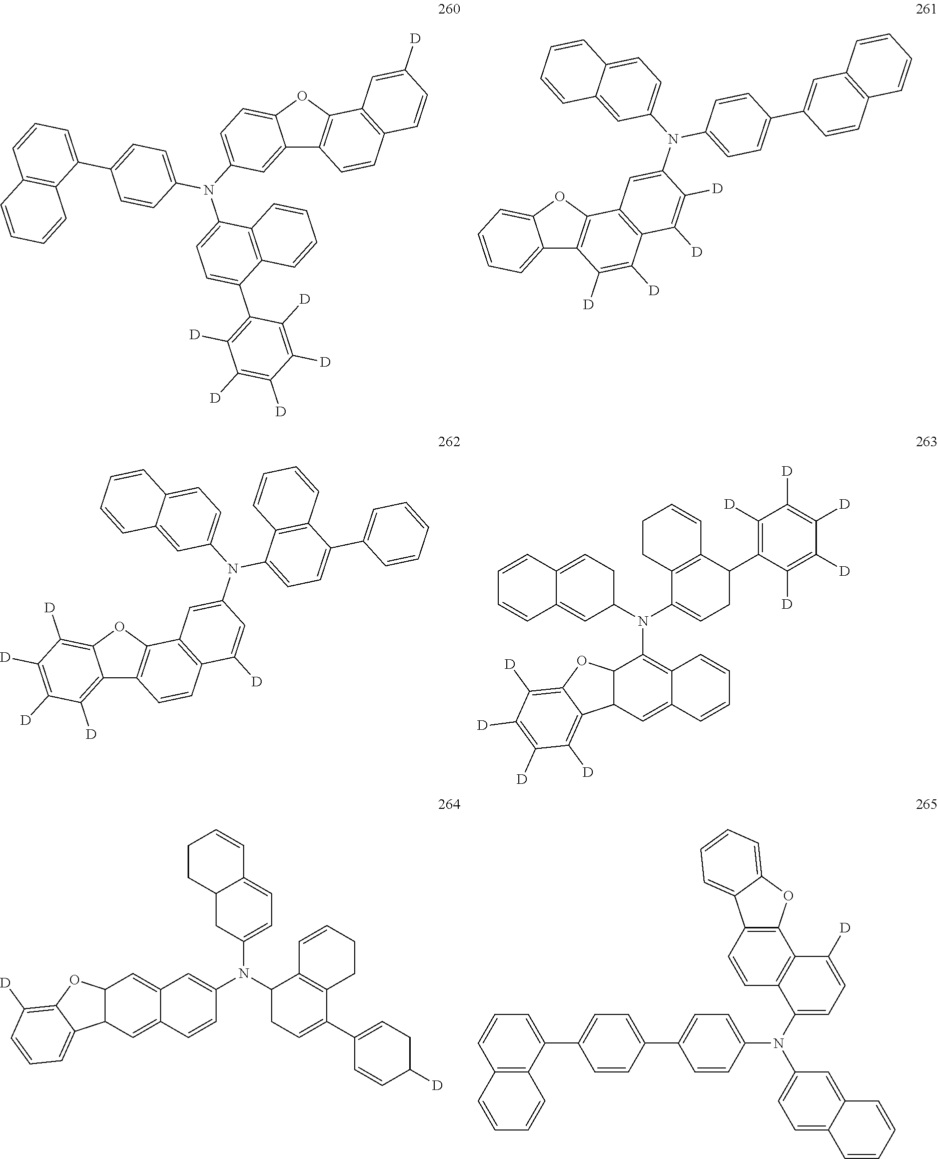

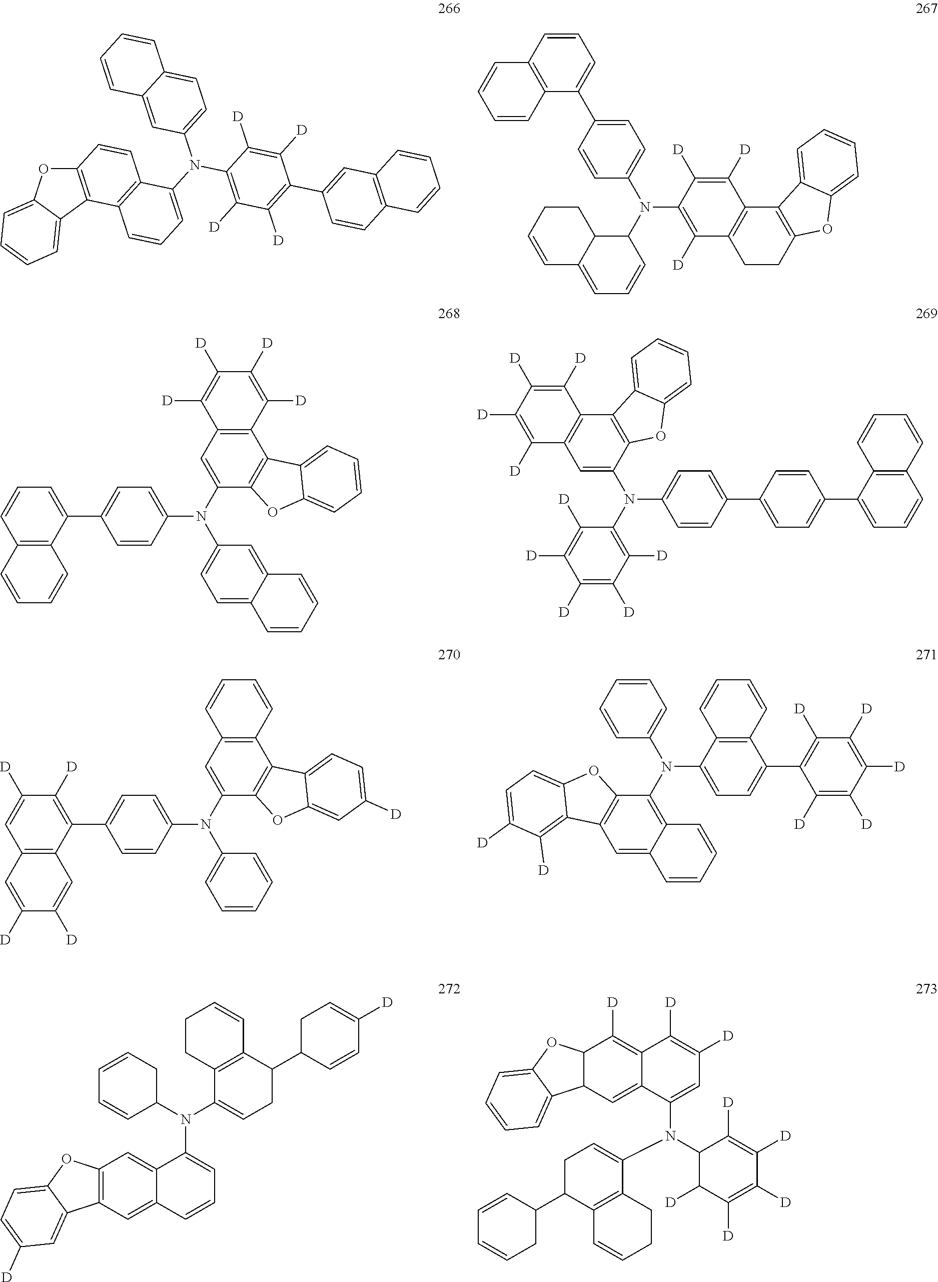

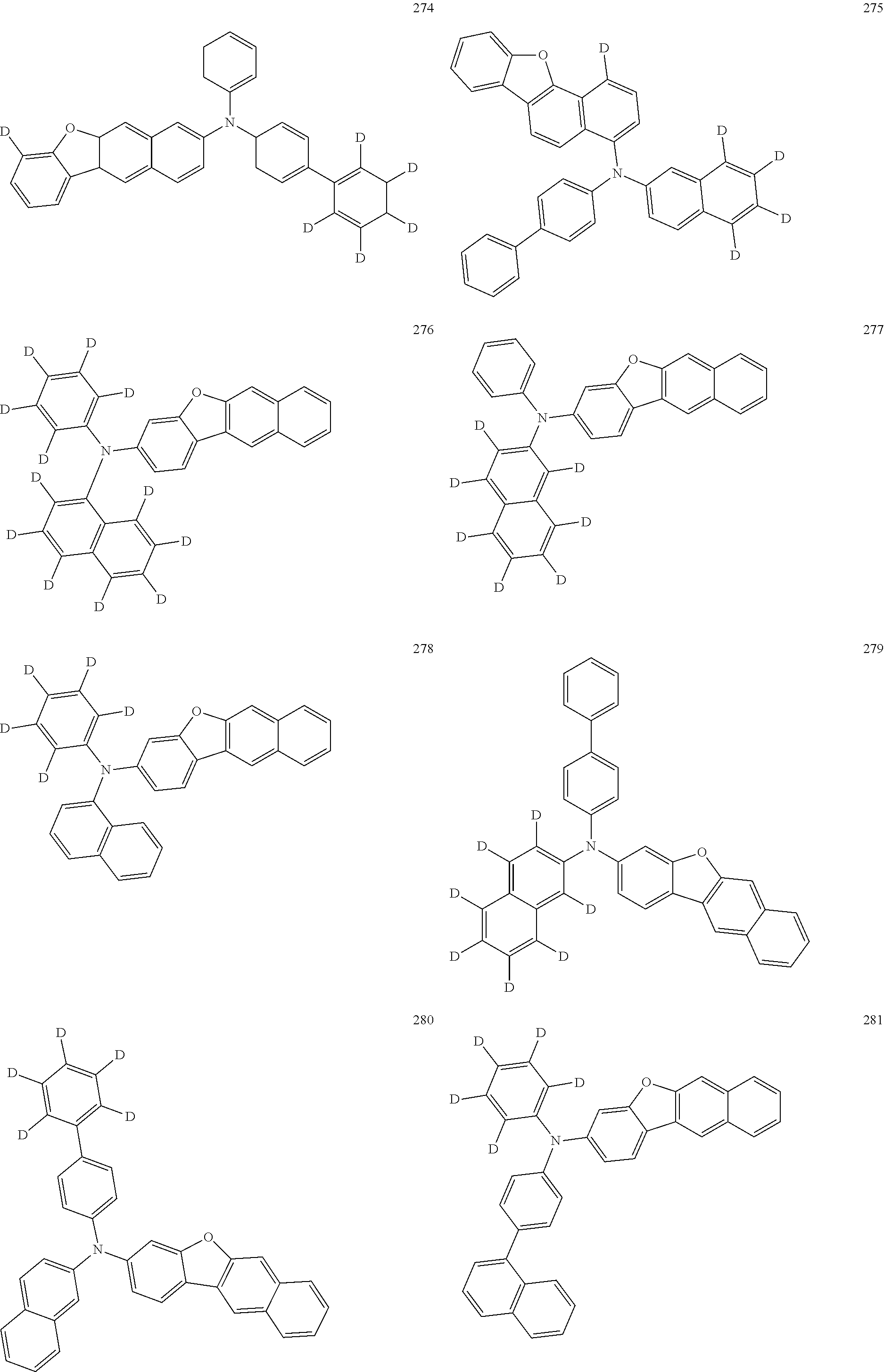

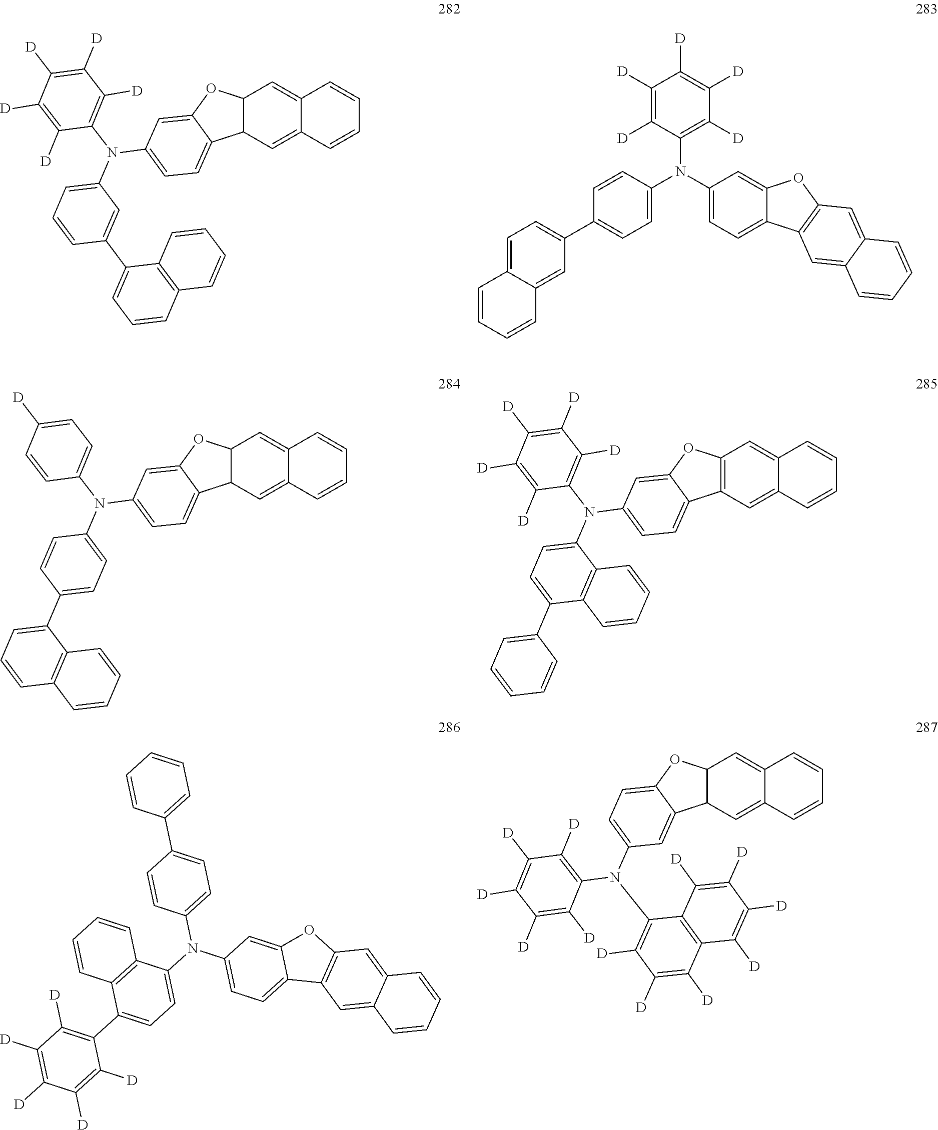

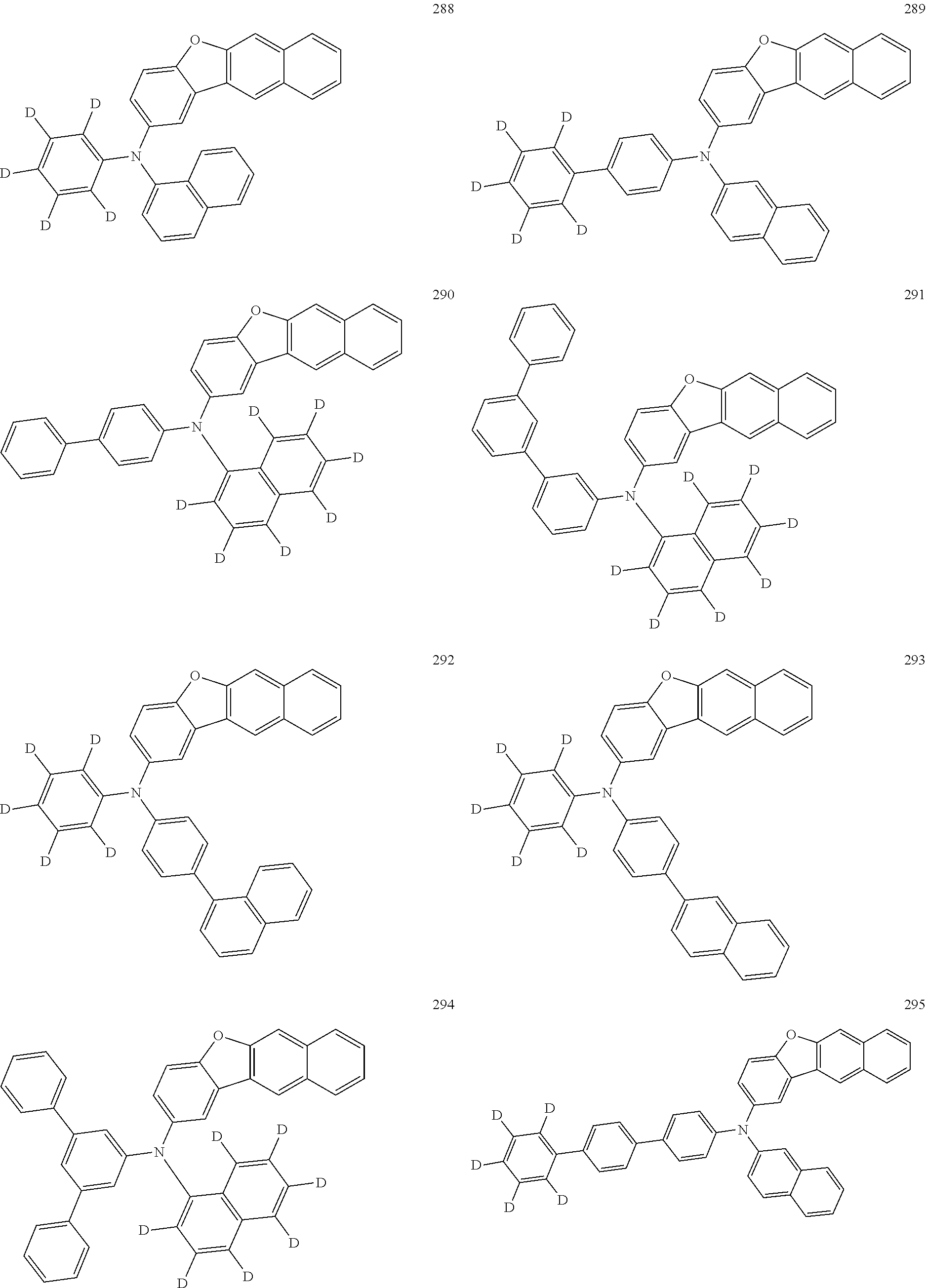

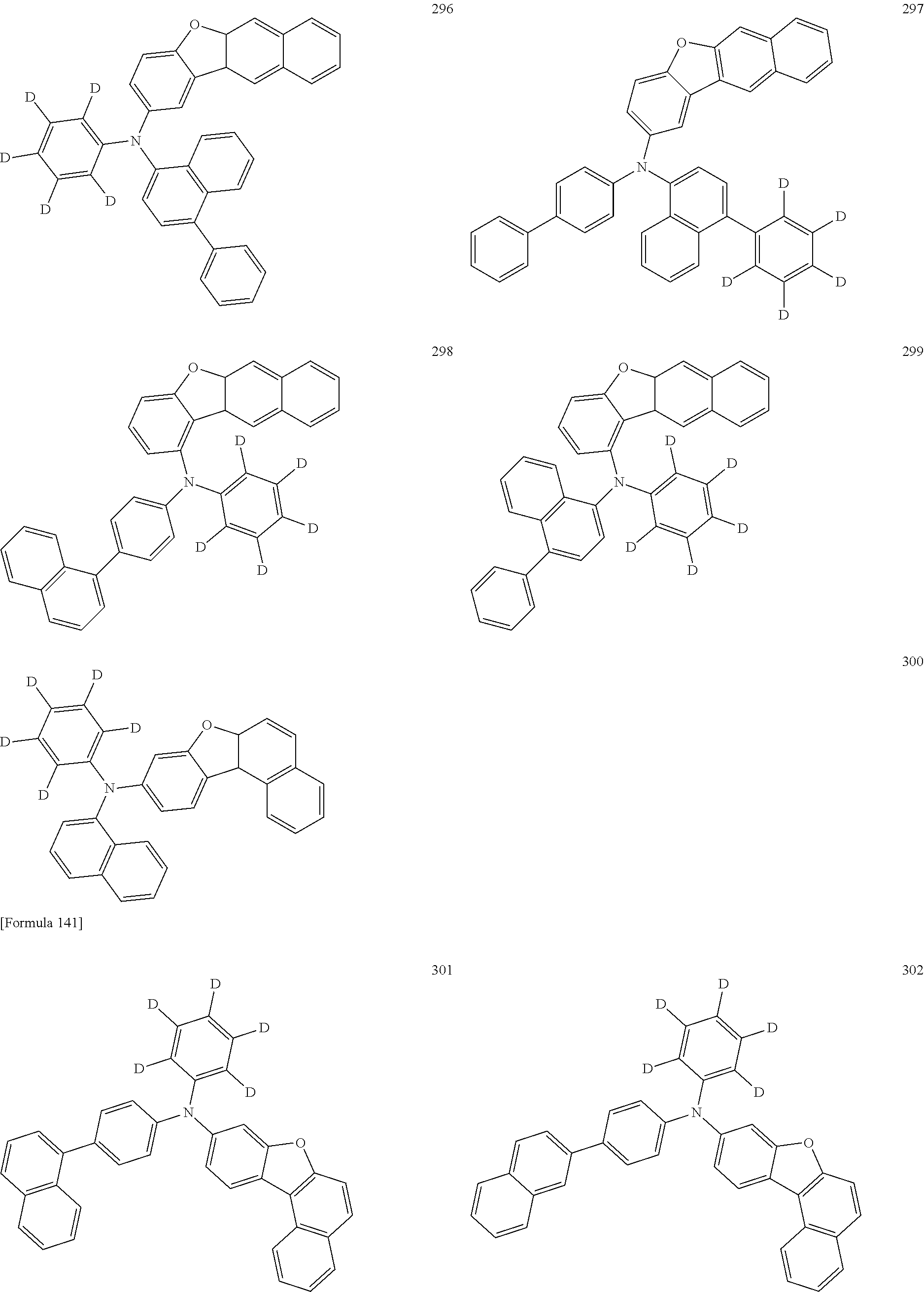

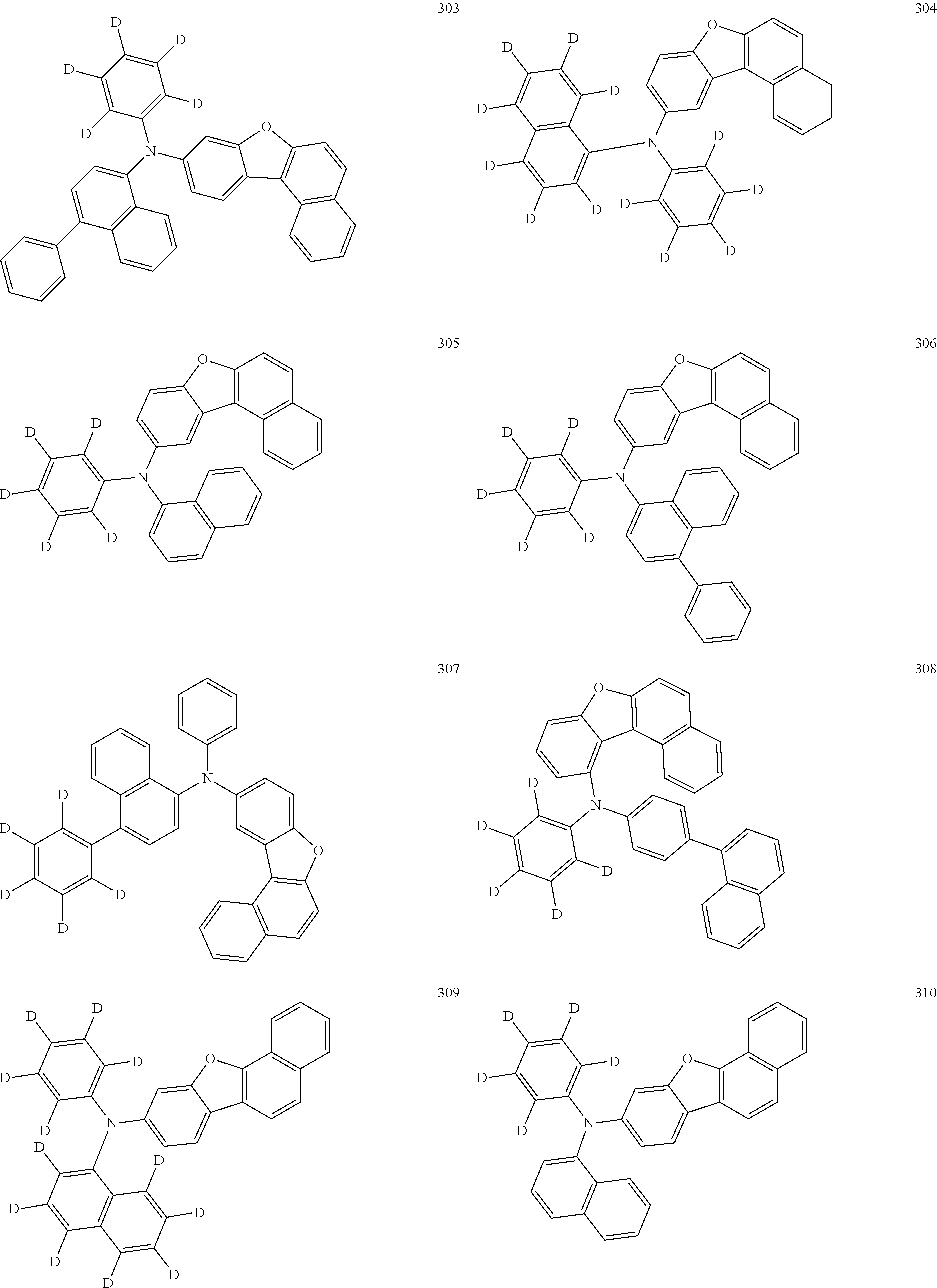

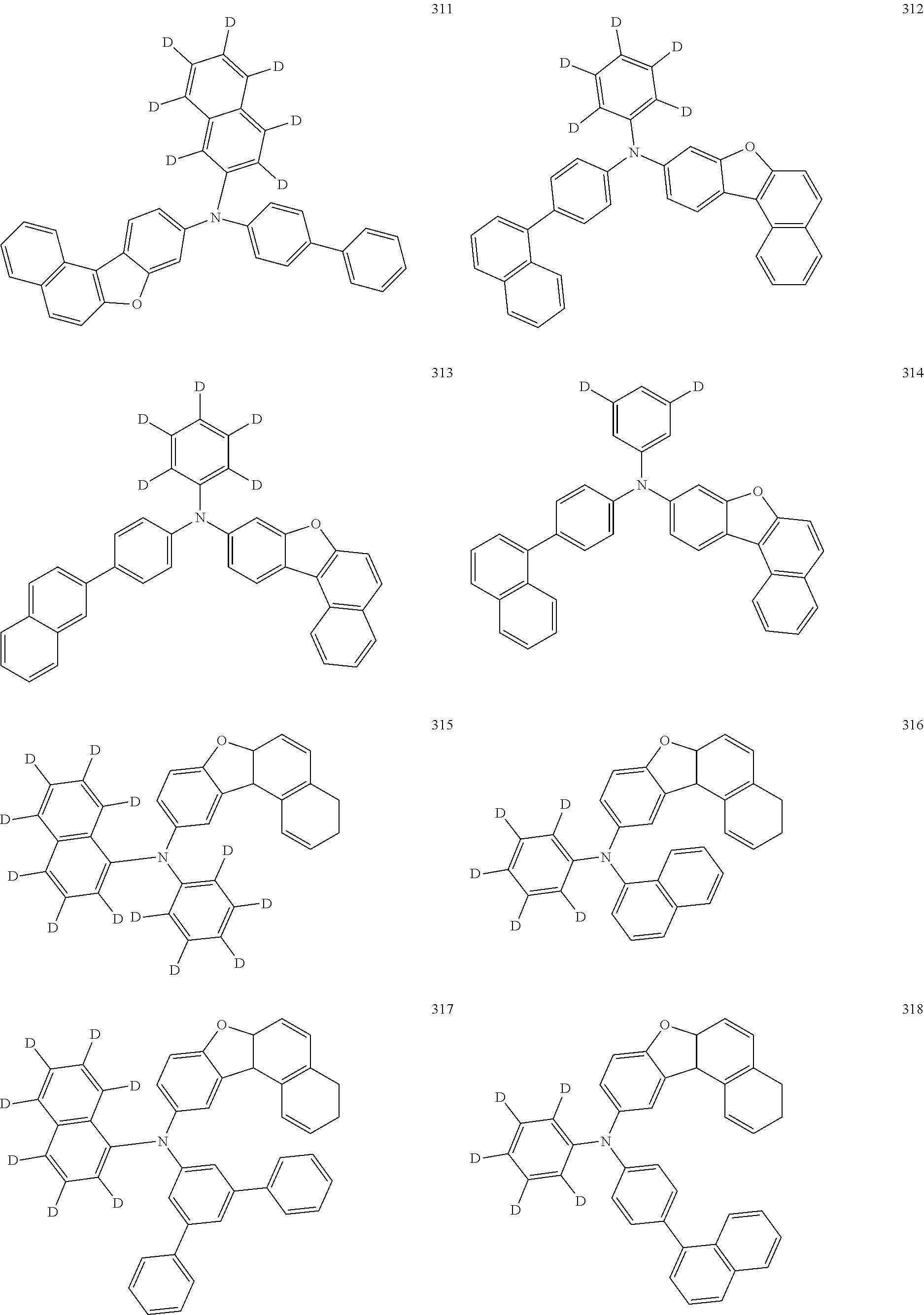

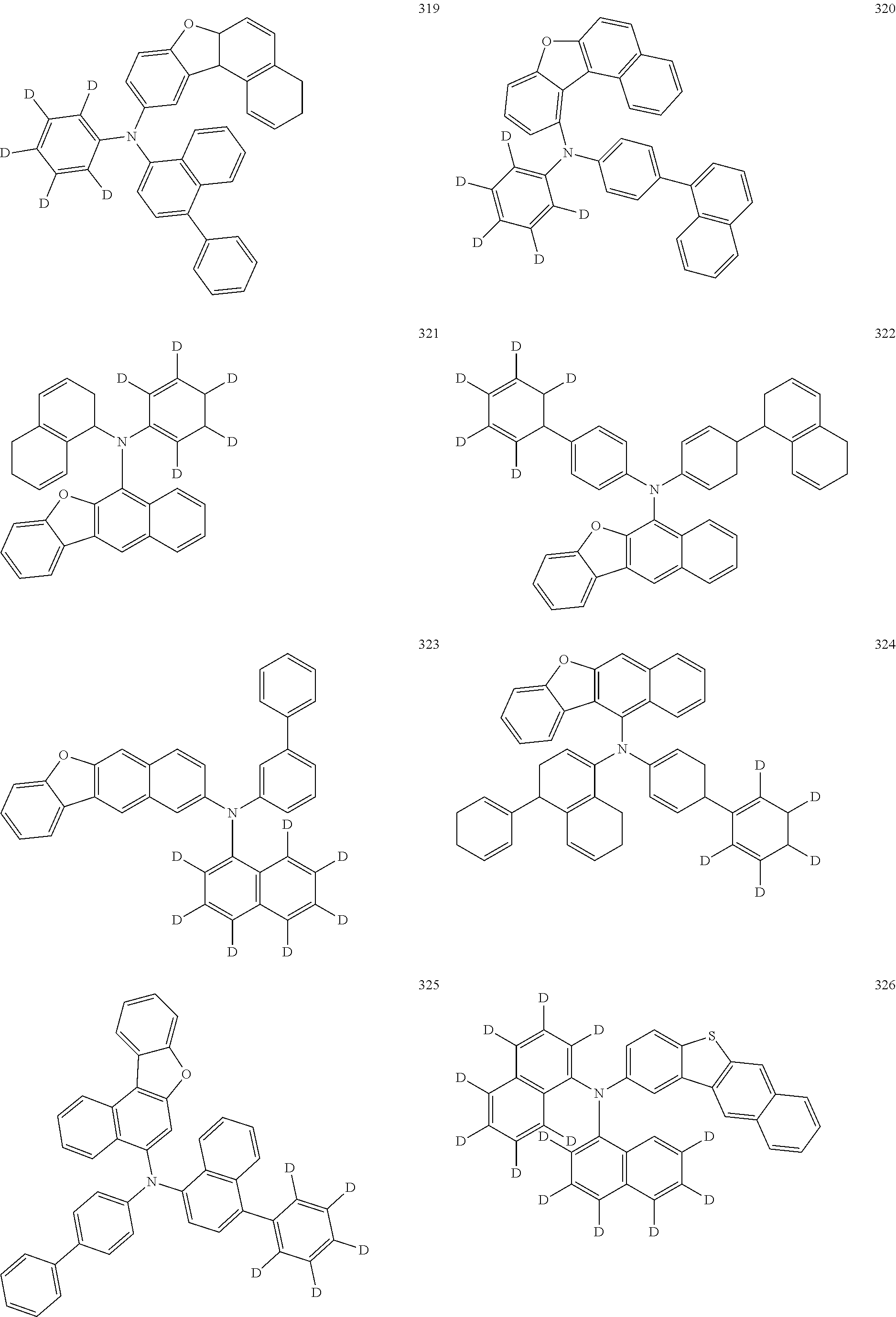

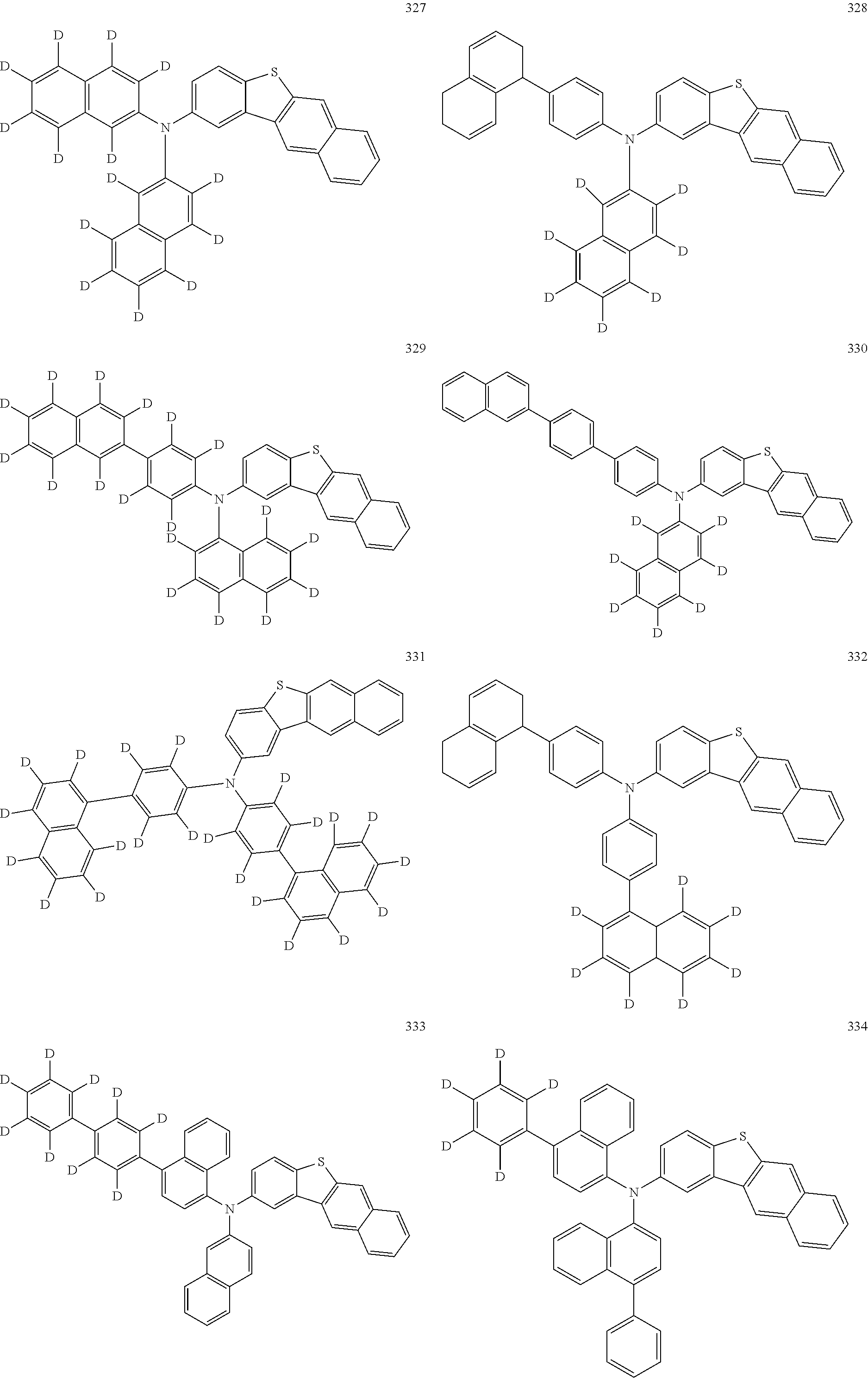

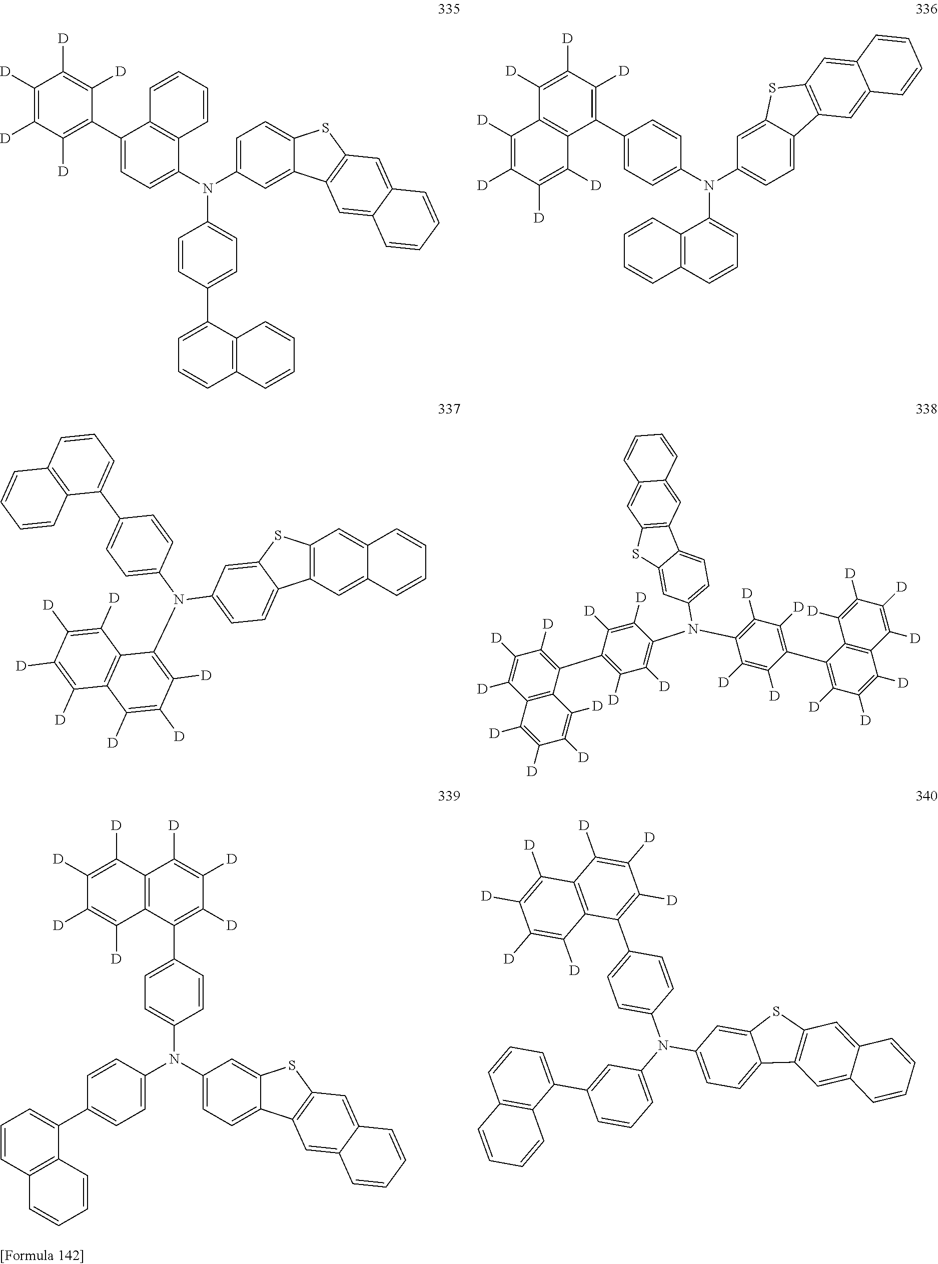

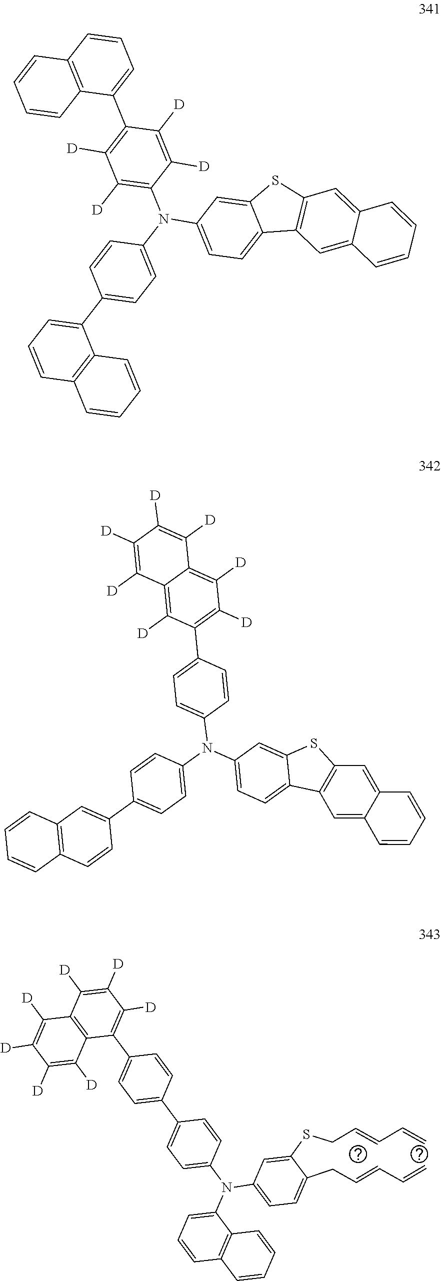 [SMILES: [2H]c1c([2H])c(N(c2ccc(-c3cccc4ccccc34)cc2)c2ccc3c(c2)sc2cc4ccccc4cc23)c([2H])c([2H])c1-c1cccc2ccccc12 ; [2H]c1c([2H])c([2H])c2c([2H])c(-c3ccc(N(c4ccc(-c5ccc6ccccc6c5)cc4)c4ccc5c(c4)sc4cc6ccccc6cc45)cc3)c([2H])c([2H])c2c1[2H] ; [2H]c1c([2H])c([2H])c2c(-c3ccc(-c4ccc(N(c5ccc(C/C=C/C=C)c(SC/C=C/C=C)c5)c5cccc6ccccc56)cc4)cc3)c([2H])c([2H])c([2H])c2c1[2H]]

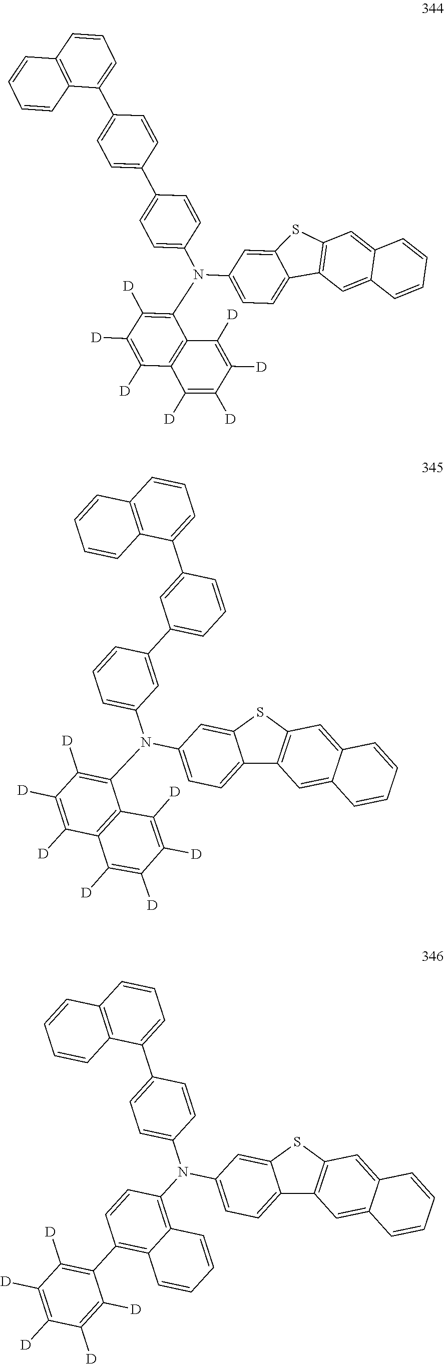 [SMILES: [2H]c1c([2H])c([2H])c2c(N(c3ccc(-c4ccc(-c5cccc6ccccc56)cc4)cc3)c3ccc4c(c3)sc3cc5ccccc5cc34)c([2H])c([2H])c([2H])c2c1[2H] ; [2H]c1c([2H])c([2H])c2c(N(c3cccc(-c4cccc(-c5cccc6ccccc56)c4)c3)c3ccc4c(c3)sc3cc5ccccc5cc34)c([2H])c([2H])c([2H])c2c1[2H] ; [2H]c1c([2H])c([2H])c(-c2ccc(N(c3ccc(-c4cccc5ccccc45)cc3)c3ccc4c(c3)sc3cc5ccccc5cc34)c3ccccc23)c([2H])c1[2H]]

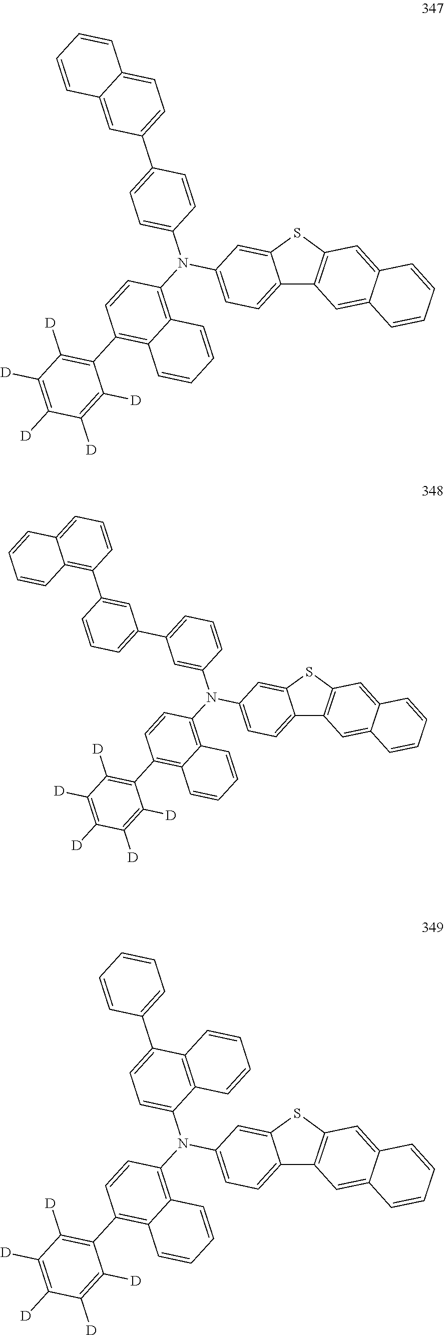 [SMILES: [2H]c1c([2H])c([2H])c(-c2ccc(N(c3ccc(-c4ccc5ccccc5c4)cc3)c3ccc4c(c3)sc3cc5ccccc5cc34)c3ccccc23)c([2H])c1[2H] ; [2H]c1c([2H])c([2H])c(-c2ccc(N(c3cccc(-c4cccc(-c5cccc6ccccc56)c4)c3)c3ccc4c(c3)sc3cc5ccccc5cc34)c3ccccc23)c([2H])c1[2H] ; [2H]c1c([2H])c([2H])c(-c2ccc(N(c3ccc4c(c3)sc3cc5ccccc5cc34)c3ccc(-c4ccccc4)c4ccccc34)c3ccccc23)c([2H])c1[2H]]

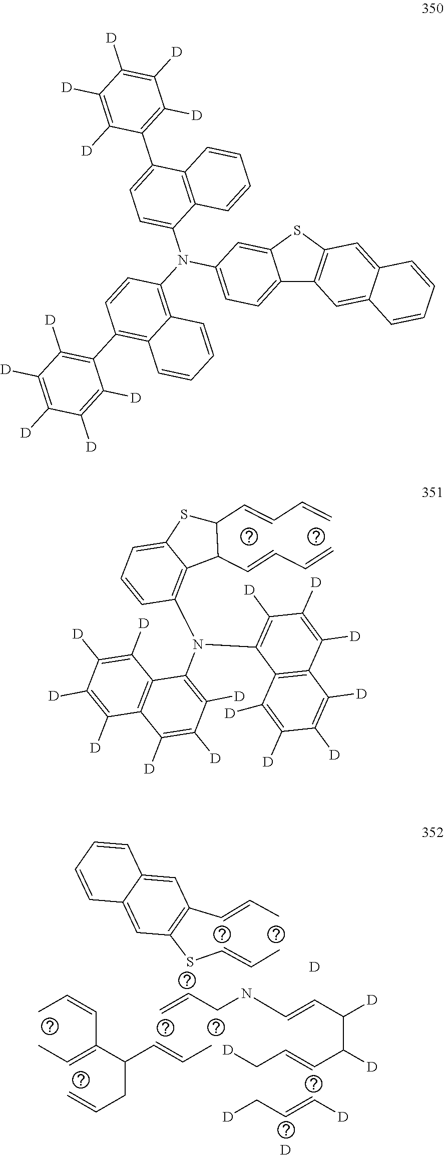

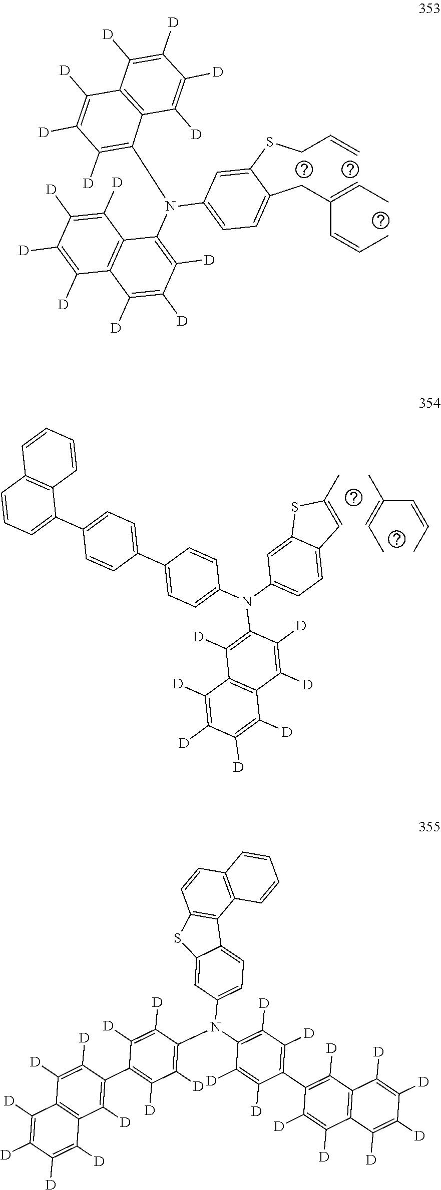

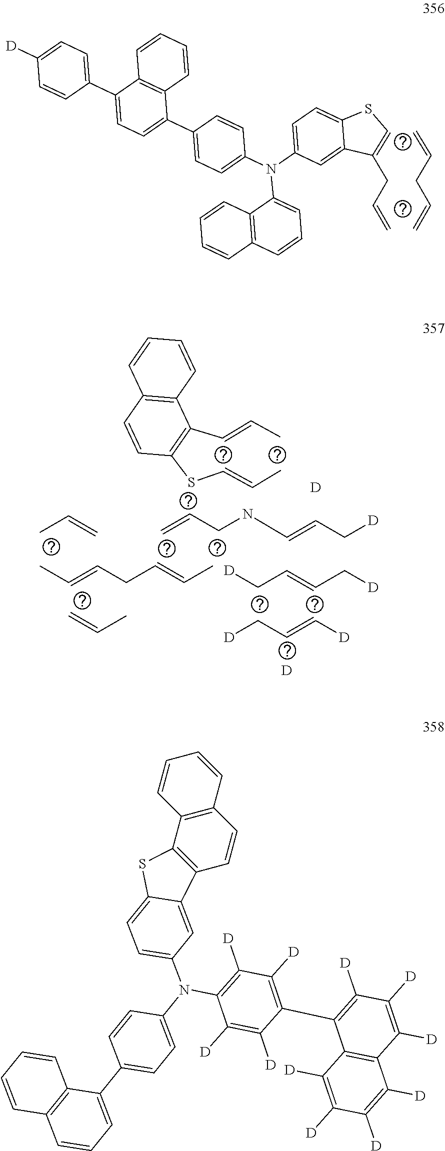

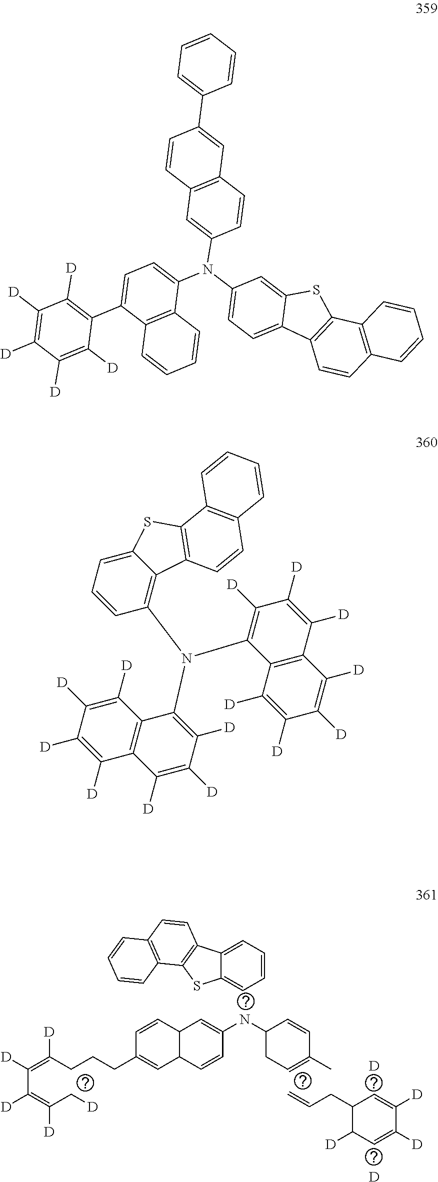

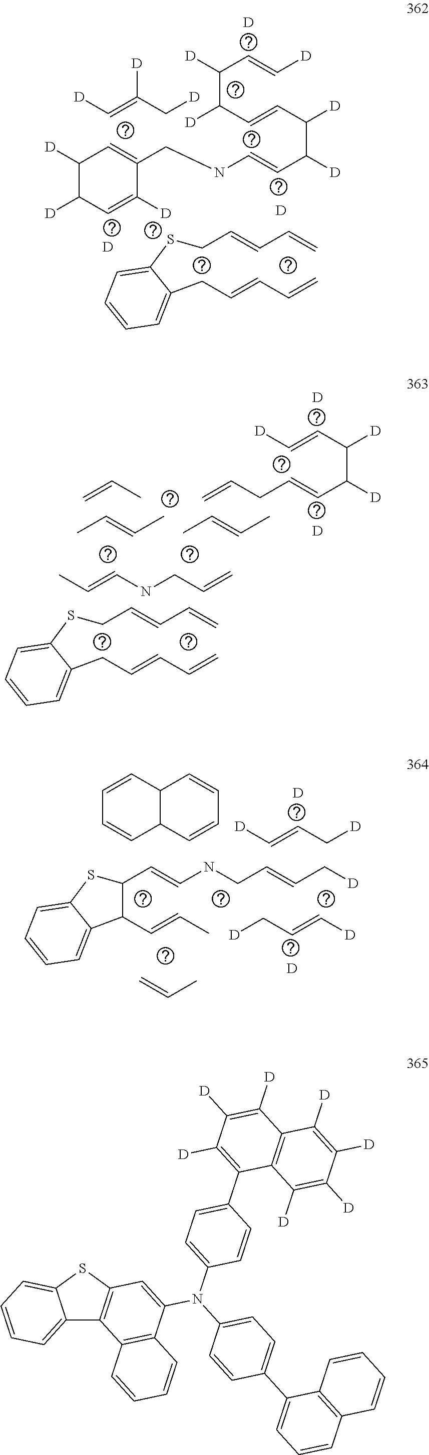

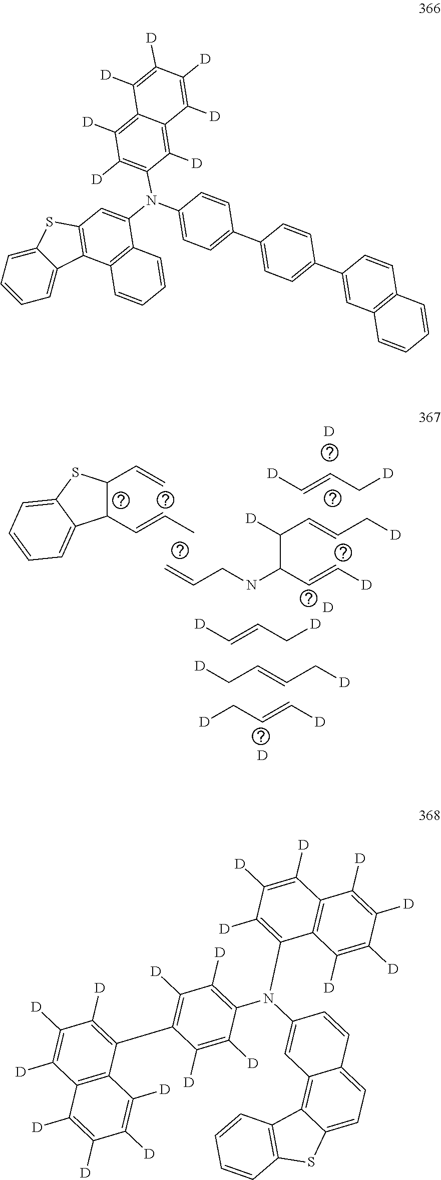

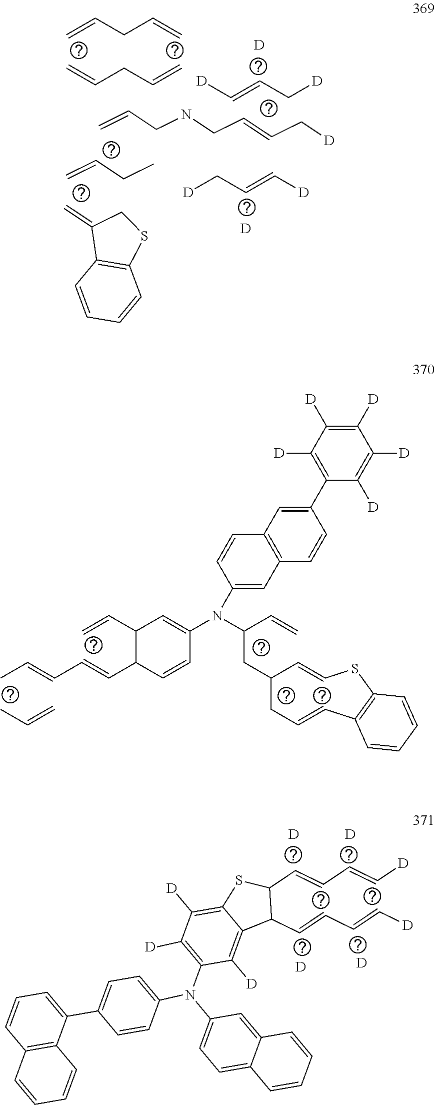

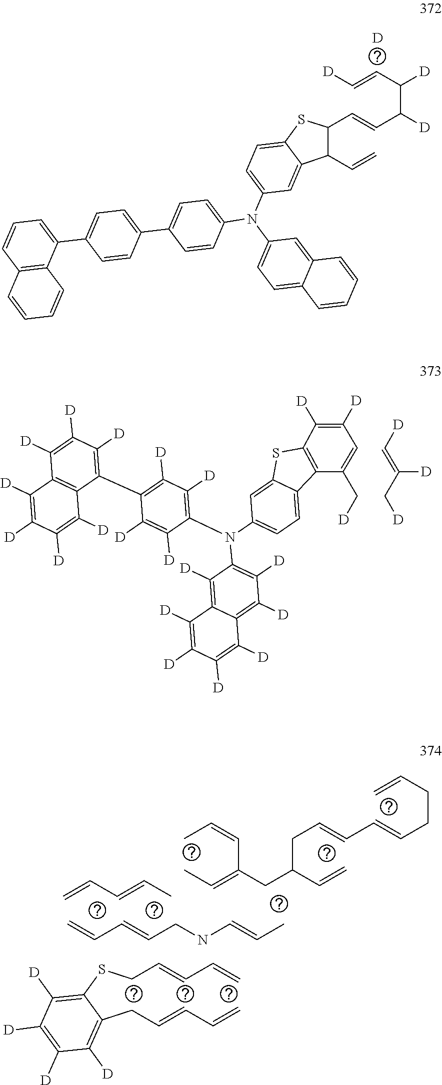

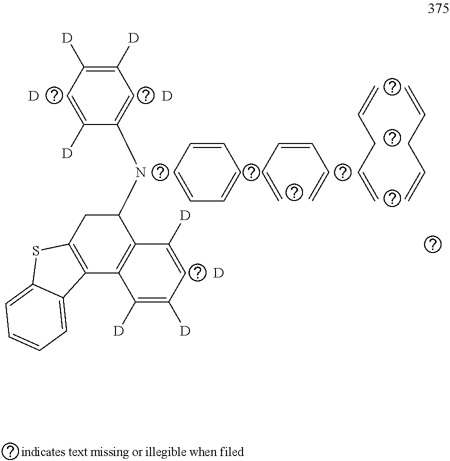

### Hole Injecting Layer

In an exemplary arrangement of the organic EL device according to the exemplary embodiment, the hole injecting layer is provided between the anode and the emitting zone.

In an exemplary arrangement of the organic EL device according to the exemplary embodiment, the anode is in direct contact with the hole injecting layer.

In an exemplary arrangement of the organic EL device according to the exemplary embodiment, the hole injecting layer is in direct contact with the hole transporting layer.

In an exemplary arrangement of the organic EL device according to the exemplary embodiment, a compound usable for the hole transporting layer (hole transporting zone material) is also usable for the hole injecting layer. In this case, the hole injecting layer preferably contains a hole transporting zone material and an acceptor material.

### Acceptor Material

The acceptor material contains at least one of a first cyclic structure represented by a formula (11) below or a second cyclic structure represented by a formula (12) below.

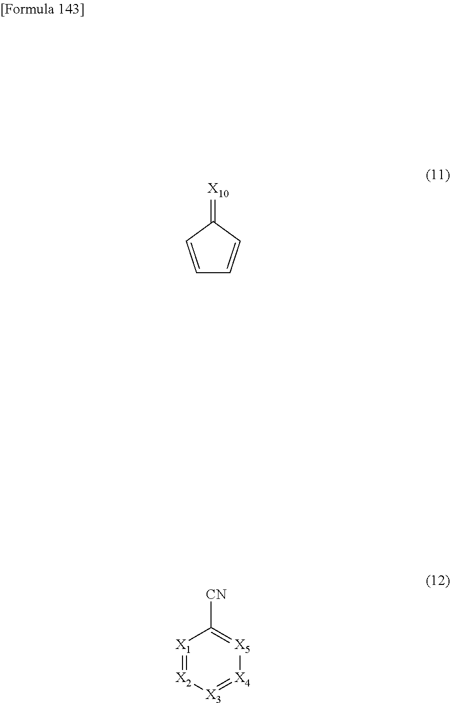

The first cyclic structure represented by the formula (11) is fused to at least one cyclic structure of a substituted or unsubstituted aromatic hydrocarbon ring having 6 to 50 ring carbon atoms or a substituted or unsubstituted heterocycle having 5 to 50 ring atoms in a molecule of the acceptor material, and
a structure represented by ═X₁₀ is represented by a formula (11a), (11b), (11c), (11d), (11e), (11f), (11g), (11h), (11i), (11j), (11k), or (11m) below.

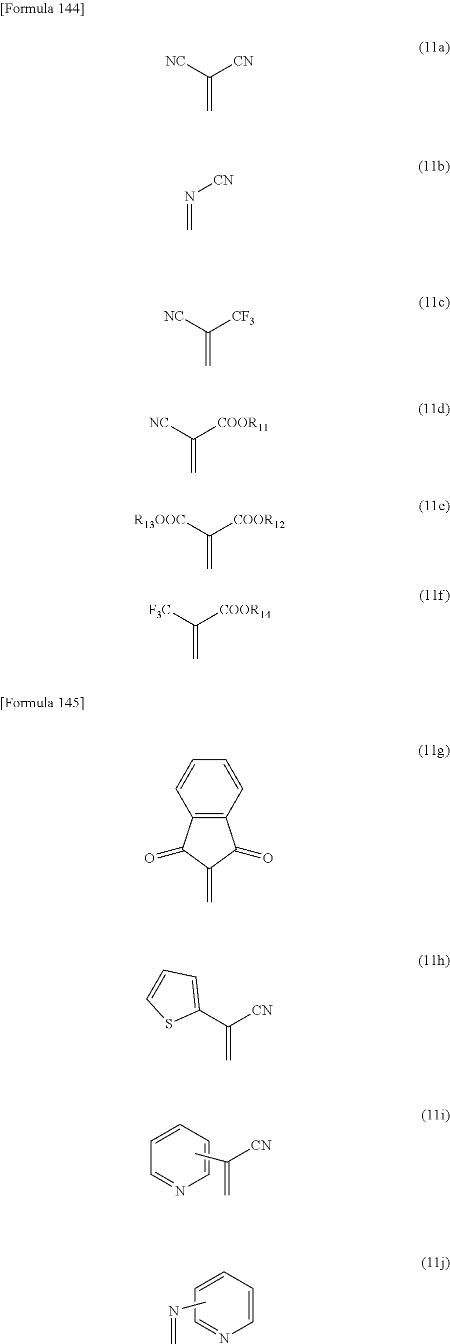

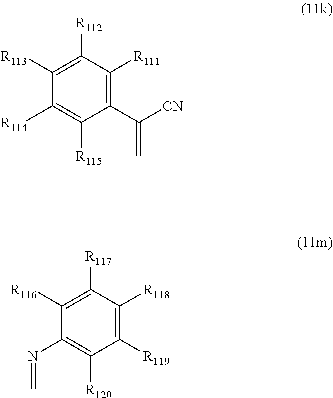

In the formula (11a), (11b), (11c), (11d), (11e), (11f), (11g), (11h), (11i), (11j), (11k) or (11m), R₁₁ to R₁₄ and R₁₁₁ to R₁₂₀ are each independently a hydrogen atom, a halogen atom, a hydroxy group, a cyano group, a substituted or unsubstituted alkyl group having 1 to 50 carbon atoms, a substituted or unsubstituted alkyl halide group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms.

In the formula (12):
X₁ to X₅ are each independently a nitrogen atom, a carbon atom bonded to R₁₅, or a carbon atom bonded to another atom in the molecule of the acceptor material;
at least one of X₁ to X₅ is a carbon atom bonded to another atom in the molecule of the acceptor material;
R₁₅ is selected from the group consisting of a hydrogen atom, a halogen atom, a cyano group, a substituted or unsubstituted alkyl group having 1 to 50 carbon atoms a substituted or unsubstituted alkyl halide group having 1 to 50 carbon atoms, a substituted or unsubstituted cycloalkyl group having 3 to 50 ring carbon atoms, a substituted or unsubstituted aryl group having 6 to 50 ring carbon atoms, a substituted or unsubstituted heterocyclic group having 5 to 50 ring atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted alkenyl group having 2 to 50 carbon atoms, a substituted or unsubstituted aralkyl group having 7 to 50 carbon atoms, a carboxy group, a substituted or unsubstituted ester group, a substituted or unsubstituted carbamoyl group, a nitro group, and a substituted or unsubstituted siloxanyl group; and
when a plurality of R₁₅ are present, the plurality of R₁₅ are mutually the same or different.

In the acceptor material, R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

In an exemplary arrangement of the organic EL device according to the exemplary embodiment, the acceptor material has at least one cyano group.

In an exemplary arrangement of the organic EL device according to the exemplary embodiment, the hole injecting layer contains the hole transporting zone material, the acceptor material and the hole transporting zone material are mutually different, and the content of the acceptor material in the hole injecting layer is less than 50 mass %.

In an exemplary arrangement of the organic EL device according to the exemplary embodiment, the content of the acceptor material in the hole injecting layer is 10 mass % or less or 5 mass % or less.

In an exemplary arrangement of the organic EL device according to the exemplary embodiment, the content of the acceptor material in the hole injecting layer is 1 mass % or more or 3 mass % or less.

In an exemplary arrangement of the organic EL device according to the exemplary embodiment, the hole transporting zone material is preferably a compound selected from the group consisting of compounds usable for the hole transporting layer described later.

In an exemplary arrangement of the organic EL device according to the exemplary embodiment, when the hole injecting layer contains the acceptor material and the hole transporting zone material, the content of the hole transporting zone material in the hole injecting layer is preferably 40 mass % or more, more preferably 45 mass % or more, and still more preferably 50 mass % or more. The content of the hole transporting zone material in the hole injecting layer is preferably 99.5 mass % or less. The total of the contents of the acceptor material and the hole transporting zone material in the hole injecting layer is 100 mass % or less.

An ester group herein is at least one group selected from the group consisting of an alkyl ester group and an aryl ester group.

An alkyl ester group herein is represented, for instance, by —C(═O)OR^{E}. R^{E} is exemplified by a substituted or unsubstituted alkyl group having 1 to 50 carbon atoms (preferably 1 to 10 carbon atoms).

An aryl ester group herein is represented, for instance, by —C(═O)OR^{Ar}. R^{Ar} is exemplified by a substituted or unsubstituted aryl group having 6 to 30 ring carbon atoms.

A siloxanyl group herein, which is a silicon compound group through an ether bond, is exemplified by a trimethylsiloxanyl group.

A carbamoyl group herein is represented by —CONH₂.

A substituted carbamoyl group herein is represented, for instance, by —CONH—Ar^{C} or —CONH—R^{C}. Ar^{C} is, for instance, at least one group selected from the group consisting of a substituted or unsubstituted aryl group having 6 to 50 ring carbon atoms (preferably 6 to 10 ring carbon atoms) and a heterocyclic group having 5 to 50 ring atoms (preferably 5 to 14 ring atoms). Ar^{C} may be a group in which a substituted or unsubstituted aryl group having 6 to 50 ring carbon atoms is bonded to a substituted or unsubstituted heterocyclic group having 5 to 50 ring atoms.

R^{C} is exemplified by a substituted or unsubstituted alkyl group having 1 to 50 carbon atoms (preferably 1 to 6 carbon atoms).

In the acceptor material, also preferably, the groups specified to be “substituted or unsubstituted” are each an “unsubstituted” group.

### Specific Examples of Acceptor Material

Specific examples of the acceptor material include the following compounds. The invention, however, is not limited to the specific examples of the acceptor material.

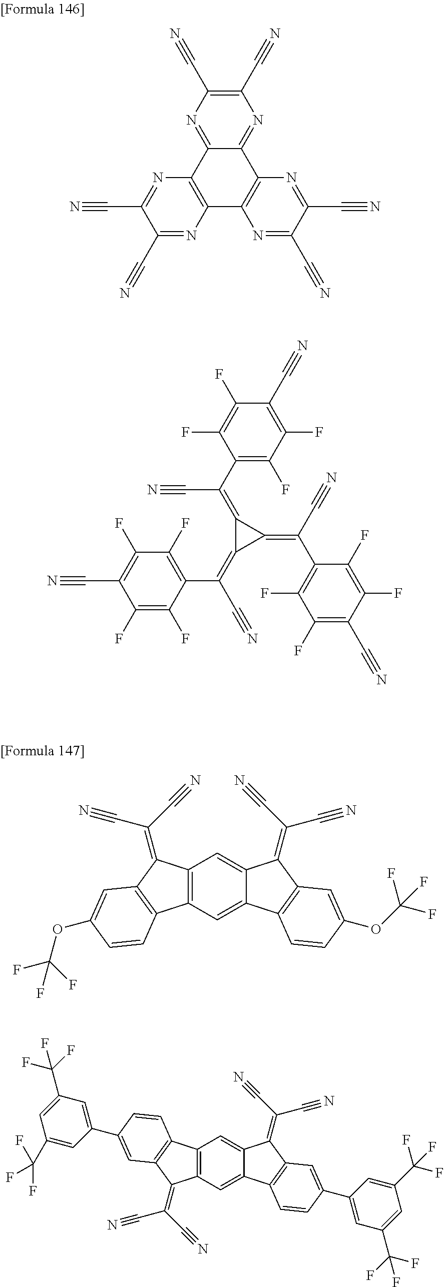 [SMILES: N#Cc1nc2c3nc(C#N)c(C#N)nc3c3nc(C#N)c(C#N)nc3c2nc1C#N ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; N#CC(C#N)=C1c2cc(OC(F)(F)F)ccc2-c2cc3c(cc21)C(=C(C#N)C#N)c1cc(OC(F)(F)F)ccc1-3 ; N#CC(C#N)=C1c2cc(-c3cc(C(F)(F)F)cc(C(F)(F)F)c3)ccc2-c2cc3c(cc21)-c1ccc(-c2cc(C(F)(F)F)cc(C(F)(F)F)c2)cc1C3=C(C#N)C#N]

### Hole Transporting Layer

In an exemplary arrangement of the organic EL device according to the exemplary embodiment, the hole transporting layer is provided between the anode and the emitting zone.

In an exemplary arrangement of the organic EL device according to the exemplary embodiment, the hole transporting zone may include one hole transporting layer or two or more hole transporting layers.

In an exemplary arrangement of the organic EL device according to the exemplary embodiment, the hole transporting layer contains a hole transporting zone material.

In an exemplary arrangement of the organic EL device according to the exemplary embodiment, the hole transporting zone material is a monoamine compound having one substituted or unsubstituted amino group in a molecule, or a diamine compound having two substituted or unsubstituted amino groups in a molecule.

In an exemplary arrangement of the organic EL device according to the exemplary embodiment, the hole transporting zone material is a monoamine compound having one substituted or unsubstituted amino group in a molecule.

In an exemplary arrangement of the organic EL device according to the exemplary embodiment, the hole transporting zone material may be the third compound represented by the formula (EB1) or at least one compound selected from the group consisting of a compound represented by a formula (C1) below and a compound represented by a formula (C2) below.

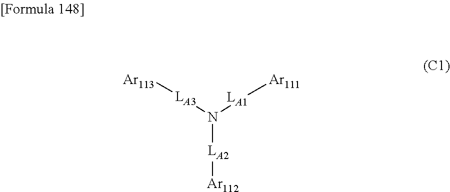

In the formula (C1):
L_{A1}, L_{A2}, and L_{A3} are each independently a single bond, a substituted or unsubstituted arylene group having 6 to 50 ring carbon atoms, or a substituted or unsubstituted divalent heterocyclic group having 5 to 50 ring atoms;
Ar₁₁₁, Ar₁₁₂, and Ar₁₁₃ are each independently a substituted or unsubstituted aryl group having 6 to 50 ring carbon atoms, a substituted or unsubstituted heterocyclic group having 5 to 50 ring atoms, or —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different; and
when a plurality of R_{C3} are present, the plurality of R_{C3} are mutually the same or different.

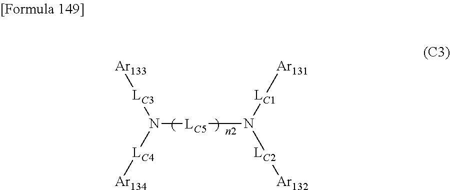

In a formula (C3) above:
L_{C1}, L_{C2}, L_{C3}, and L_{C4} are each independently a single bond, a substituted or unsubstituted arylene group having 6 to 50 ring carbon atoms, or a substituted or unsubstituted divalent heterocyclic group having 5 to 50 ring atoms;
n2 is 1, 2, 3, or 4;
when n2 is 1, L_{C5} is a substituted or unsubstituted arylene group having 6 to 50 ring carbon atoms, or a substituted or unsubstituted divalent heterocyclic group having 5 to 50 ring atoms;
when n2 is 2, 3, or 4, a plurality of L_{C5} are mutually the same or different;
when n2 is 2, 3, or 4, a plurality of L_{C5} are mutually bonded to form a substituted or unsubstituted monocyclic ring, mutually bonded to form a substituted or unsubstituted fused ring, or not mutually bonded;
L_{C5} forming neither the substituted or unsubstituted monocyclic ring nor the substituted or unsubstituted fused ring is a substituted or unsubstituted arylene group having 6 to 50 ring carbon atoms, or a substituted or unsubstituted divalent heterocyclic group having 5 to 50 ring atoms;
Ar₁₃₁, Ar₁₃₂, Ar₁₃₃, and Ar₁₃₄ are each independently a substituted or unsubstituted aryl group having 6 to 50 ring carbon atoms, a substituted or unsubstituted heterocyclic group having 5 to 50 ring atoms, or —Si(R_{C1})(R_{C2})(R_{C3});
R_{C1}, R_{C2}, and R_{C3} are each independently a substituted or unsubstituted aryl group having 6 to 50 ring carbon atoms;
when a plurality of R_{C1} are present, the plurality of R_{C1} are mutually the same or different;
when a plurality of R_{C2} are present, the plurality of R_{C2} are mutually the same or different; and
when a plurality of R_{C3} are present, the plurality of R_{C3} are mutually the same or different.

In an exemplary composition according to the exemplary embodiment, a first amino group represented by a formula (C3-1) below and a second amino group represented by a formula (C3-2) below in a compound represented by the formula (C3) are an identical group.

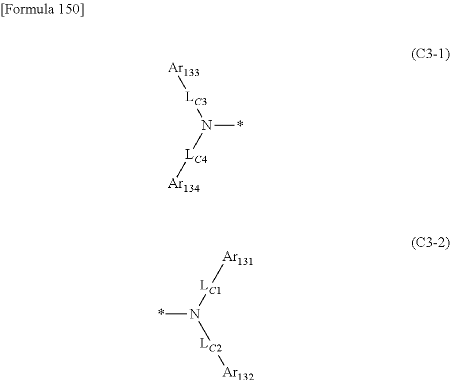

In the formulae (C3-1) and (C3-2), * each represent a bonding position to L_{C5}.

In an exemplary arrangement of the organic EL device according to the exemplary embodiment, the first amino group represented by the formula (C3-1) and the second amino group represented by the formula (C3-2) may be mutually different compounds.

### Specific Examples of Hole Transporting Zone Material

Specific examples of the hole transporting zone material include the following compounds. It should however be noted that the invention is not limited to the specific examples of the hole transporting zone material.

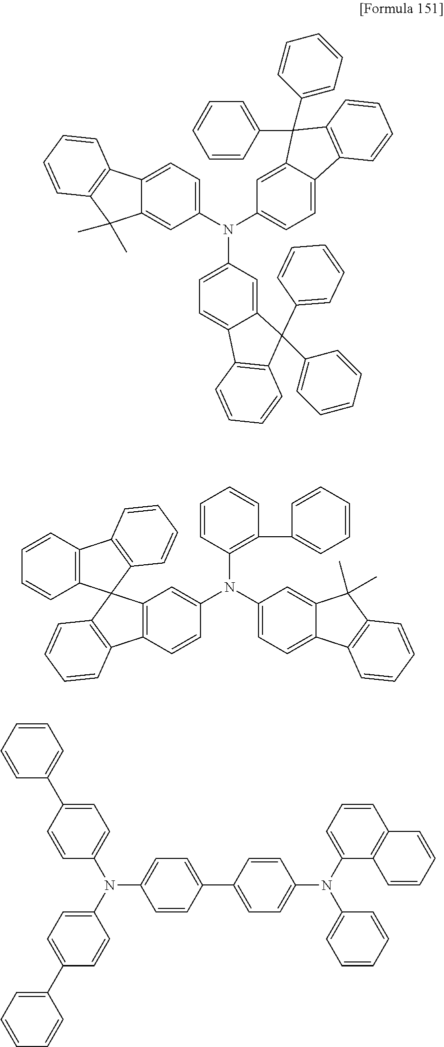

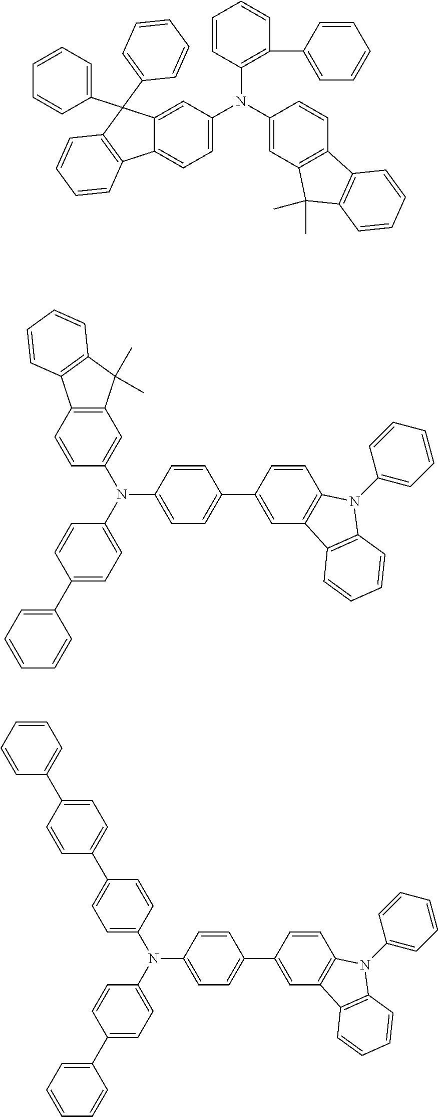 [SMILES: CC1(C)c2ccccc2-c2ccc(N(c3ccc4c(c3)C(c3ccccc3)(c3ccccc3)c3ccccc3-4)c3ccccc3-c3ccccc3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1]

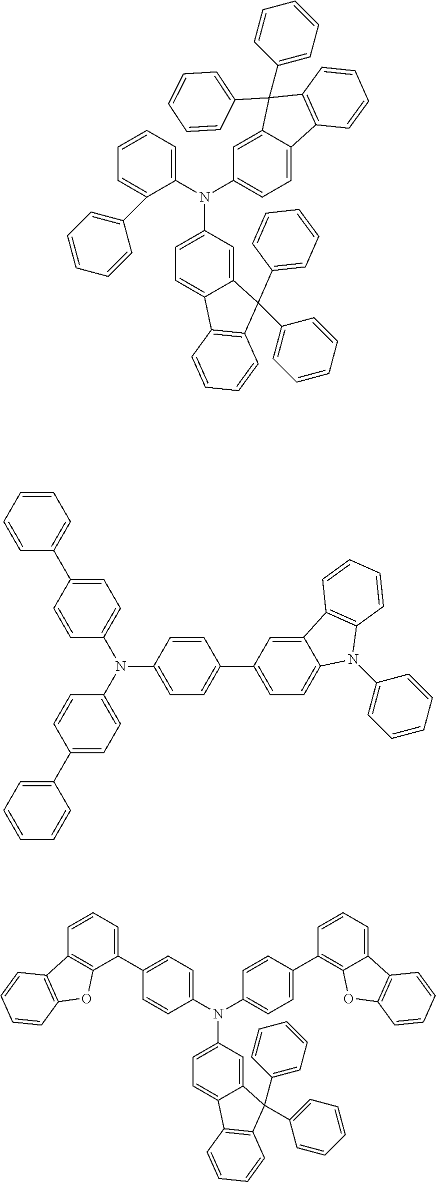 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc(-c4ccccc4)cc3)c3ccc(-c4ccc5c(c4)c4ccccc4n5-c4ccccc4)cc3)cc2)cc1 ; c1ccc(C2(c3ccccc3)c3ccccc3-c3ccc(N(c4ccc(-c5cccc6c5oc5ccccc56)cc4)c4ccc(-c5cccc6c5oc5ccccc56)cc4)cc32)cc1]

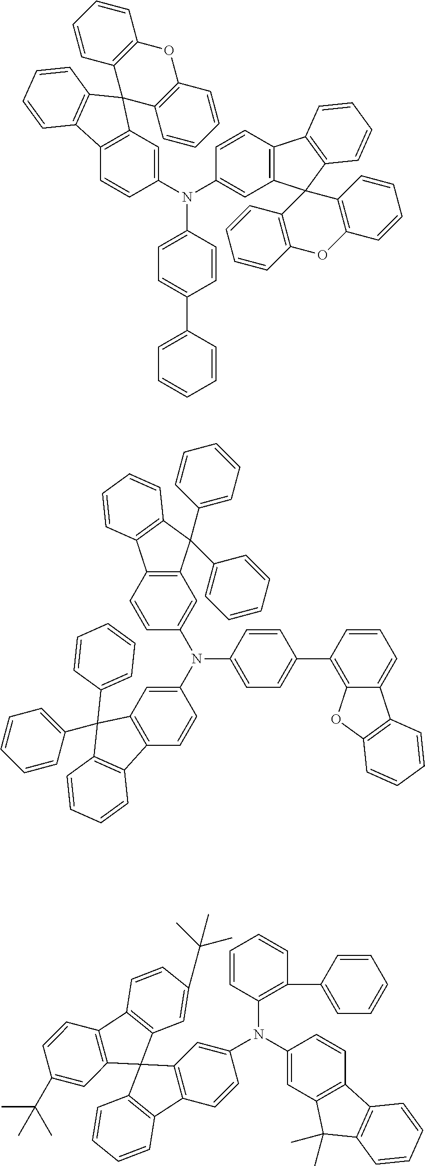 [SMILES: c1ccc(-c2ccc(N(c3ccc4c(c3)C3(c5ccccc5Oc5ccccc53)c3ccccc3-4)c3ccc4c(c3)C3(c5ccccc5Oc5ccccc53)c3ccccc3-4)cc2)cc1 ; c1ccc(C2(c3ccccc3)c3ccccc3-c3ccc(N(c4ccc(-c5cccc6c5oc5ccccc56)cc4)c4ccc5c(c4)C(c4ccccc4)(c4ccccc4)c4ccccc4-5)cc32)cc1 ; CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2]

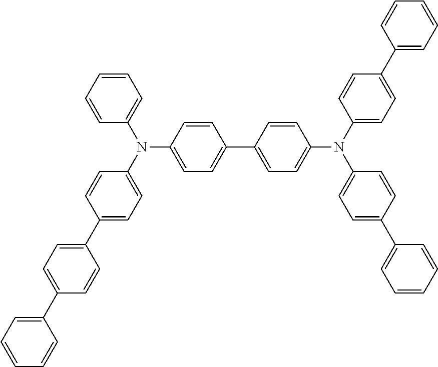 [SMILES: c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

### Electron Blocking Layer

Preferably, the electron blocking layer permits transport of holes and blocks electrons from reaching a layer provided closer to the anode (e.g., the hole transporting layer) beyond the electron blocking layer.

In an exemplary arrangement of the organic EL device of the exemplary embodiment, the electron blocking layer contains the third compound represented by the formula (EB1).

In addition to the third compound represented by the formula (EB1), examples of the compound contained in the electron blocking layer of the organic EL device of the exemplary embodiment include a well-known compound used for the electron blocking layer, which may be at least one compound selected from the group consisting of an aromatic amine compound and a carbazole derivative. The compound contained in the electron blocking layer may be a monoamine compound having one substituted or unsubstituted amino group in a molecule. Further, the compound contained in the electron blocking layer may have, in a molecule, a substituted or unsubstituted carbazolyl group and one substituted or unsubstituted amino group.

In order to prevent excitation energy from leaking out from the emitting layer toward neighboring layer(s), the electron blocking layer may block excitons generated in the emitting layer from being transferred to a layer(s) closer to the anode (e.g., the hole transporting layer and the hole injecting layer) beyond the electron blocking layer.

### Emitting Zone

The emitting zone of the organic EL device according to the exemplary embodiment includes at least two emitting layers. The emitting zone of the organic EL device according to the exemplary embodiment includes the first emitting layer and the second emitting layer.

In an exemplary arrangement of the organic EL device of the exemplary embodiment, the first emitting layer and the second emitting layer are in direct contact with each other.

Herein, a layer arrangement in which “the first emitting layer and the second emitting layer are in direct contact with each other” may include one of embodiments (LS1), (LS2), and (LS3) below.

(LS1) An embodiment in which a region containing both the first host material and the second host material is generated in a process of vapor-depositing the compound of the first emitting layer and vapor-depositing the compound of the second emitting layer, and is present on the interface between the first emitting layer and the second emitting layer.

(LS2) An arrangement in which in a case of containing a luminescent compound in the first emitting layer and the second emitting layer, a region containing the first host material, the second host material and the luminescent compound is generated in a process of vapor-depositing the compound of the first emitting layer and vapor-depositing the compound of the second emitting layer, and is present on the interface between the first emitting layer and the second emitting layer.

(LS3) An arrangement in which in a case of containing a luminescent compound in the first emitting layer and the second emitting layer, a region containing the luminescent compound, a region containing the first host material, or a region containing the second host material is generated in a process of vapor-depositing the compound of the first emitting layer and vapor-depositing the compound of the second emitting layer, and is present on the interface between the first emitting layer and the second emitting layer.

In an exemplary arrangement of the organic EL device of the exemplary embodiment, the first emitting layer is disposed between the anode and the second emitting layer. In this case, the first emitting layer is preferably in direct contact with the organic layer that contains the third compound represented by the formula (EB1).

In an exemplary arrangement of the organic EL device of the exemplary embodiment, the second emitting layer is disposed between the anode and the first emitting layer.

In an exemplary arrangement of the organic EL device of the exemplary embodiment, among a plurality of layers in the emitting zone, one of the first emitting layer and the second emitting layer is disposed closest to the anode.

In an exemplary arrangement of the organic EL device of the exemplary embodiment, one of the first emitting layer and the second emitting layer is disposed closest to the cathode among the plurality of layers of the emitting zone.

The organic EL device according to the exemplary embodiment may include the anode, the first emitting layer, the second emitting layer, and the cathode in this order, or the order of the first emitting layer and the second emitting layer may be reversed. In other words, the organic EL device may include the anode, the second emitting layer, the first emitting layer, and the cathode in this order. Regardless of the order of the first emitting layer and the second emitting layer, the effect of a layered structure of the first emitting layer and the second emitting layer is expected to be exhibited by selecting a combination of materials satisfying the relationship of the numerical formula (Numerical Formula 1A).

### First Emitting Layer

The first emitting layer contains the first host material and the first luminescent compound that emits light having a maximum peak wavelength of 500 nm or less. The first host material and the second host material are different from each other.

Herein, the host material refers to, for instance, a material that accounts for 50 mass % or more of the layer. For instance, the first emitting layer contains 50 mass % or more of a compound as the first host material with respect to the total mass of the first emitting layer. The compound as the first host material is occasionally referred to as a first compound. For instance, the second emitting layer contains 50 mass % or more of a compound as the second host material with respect to the total mass of the second emitting layer. The compound as the second host material is occasionally referred to as a second compound. Alternatively, for instance, the host material may account for 60 mass % or more of the layer, 70 mass % or more of the layer, 80 mass % or more of the layer, 90 mass % or more of the layer, or 95 mass % or more of the layer.

In the organic EL device according to the exemplary embodiment, the first luminescent compound and the second luminescent compound are mutually the same or different.

In an exemplary arrangement of the organic EL device of the exemplary embodiment, the first luminescent compound emits light having a maximum peak wavelength of 480 nm or less.

In an exemplary arrangement of the organic EL device of the exemplary embodiment, the first luminescent compound emits light having a maximum peak wavelength of 430 nm or more.

In an exemplary arrangement of the organic EL device of the exemplary embodiment, the first luminescent compound emits fluorescence having a maximum peak wavelength of 500 nm or less, or emits fluorescence having a maximum peak wavelength of 480 nm or less.

In an exemplary arrangement of the organic EL device of the exemplary embodiment, the first luminescent compound emits fluorescence having a maximum peak wavelength of 430 nm or more.

In an exemplary arrangement of the organic EL device of the exemplary embodiment, the first luminescent compound is a compound containing no azine ring structure in a molecule.

In an exemplary arrangement of the organic EL device of the exemplary embodiment, the first luminescent compound is not a boron-containing complex. In an exemplary arrangement of the organic EL device of the exemplary embodiment, the first luminescent compound is not a complex.

In an exemplary arrangement of the organic EL device of the exemplary embodiment, the first emitting layer contains no metal complex. In an exemplary arrangement of the organic EL device of the exemplary embodiment, the first emitting layer contains no boron-containing complex.

In an exemplary arrangement of the organic EL device of the exemplary embodiment, the first emitting layer contains no phosphorescent material (dopant material).

In an exemplary arrangement of the organic EL device of the exemplary embodiment, the first emitting layer contains neither a heavy-metal complex nor a phosphorescent rare earth metal complex. Examples of the heavy-metal complex herein include an iridium complex, osmium complex, and platinum complex.

A method of measuring the maximum peak wavelength of the compound is as follows. A toluene solution of a measurement target compound at a concentration of 5 μmol/L is prepared and put in a quartz cell. An emission spectrum (ordinate axis: luminous intensity, abscissa axis: wavelength) of the thus-obtained sample is measured at a normal temperature (300K). The emission spectrum can be measured using a spectrophotometer (apparatus name: F-7000) produced by Hitachi High-Tech Science Corporation. It should be noted that the apparatus for measuring the emission spectrum is not limited to the apparatus used herein.

A peak wavelength of the emission spectrum exhibiting the maximum luminous intensity is defined as the maximum peak wavelength. Herein, the maximum peak wavelength of fluorescence is occasionally referred to as a maximum fluorescence peak wavelength (FL-peak).

In an exemplary arrangement of the organic EL device of the exemplary embodiment, assuming that a peak exhibiting a maximum luminous intensity in an emission spectrum of the first luminescent compound is defined as a maximum peak and a height of the maximum peak is defined as 1, heights of other peaks appearing in the emission spectrum are less than 0.6. It should be noted that the peaks in the emission spectrum are defined as local maximum values.

In an exemplary arrangement of the organic EL device of the exemplary embodiment, the number of peaks in the emission spectrum of the first luminescent compound is preferably less than three.

In an exemplary arrangement of the organic EL device of the exemplary embodiment, the triplet energy of the first host material T₁(H1) and a triplet energy of the first luminescent compound T₁(D1) satisfy a relationship of a numerical formula (Numerical Formula 6) below.

$${T}_{1} ( D 1 ) > {T}_{1} ( H 1 ) \qquad ( Numerical Formula 6 )$$

When the first host material and the first luminescent compound satisfy the relationship of the numerical formula (Numerical Formula 6), triplet excitons generated in the first emitting layer are transferred not onto the first luminescent compound having higher triplet energy but onto the first host material, thereby being easily transferred to the second emitting layer.

In an exemplary arrangement of the organic EL device of the exemplary embodiment, a singlet energy of the first host material S₁(H1) and a singlet energy of the first luminescent compound S₁(D1) satisfy a relationship of a numerical formula (Numerical Formula 5) below. The singlet energy S₁ means an energy difference between the lowest singlet state and the ground state.

$${S}_{1} ( H 1 ) > {S}_{1} ( D 1 ) \qquad ( Numerical Formula 5 )$$

When the first host material and the first luminescent compound satisfy the relationship of the numerical formula (Numerical Formula 5), singlet excitons generated on the first host material easily energy-transfer from the first host material to the first luminescent compound, thereby contributing to the fluorescence of the first luminescent compound.

### Triplet Energy T₁

A method of measuring a triplet energy T₁ is exemplified by a method below.

A measurement target compound is dissolved in EPA (diethylether:isopentane:ethanol=5:5:2 in volume ratio) so as to fall within a range from 10⁻⁵ mol/L to 10⁻⁴ mol/L to prepare a solution, and the obtained solution is encapsulated in a quartz cell to provide a measurement sample. A phosphorescence spectrum (ordinate axis: phosphorescent luminous intensity, abscissa axis: wavelength) of the measurement sample is measured at a low temperature (77K). A tangent is drawn to the rise of the phosphorescence spectrum close to the short-wavelength region. An energy amount is calculated by a conversion equation (F1) below on a basis of a wavelength value λ_{edge} [nm] at an intersection of the tangent and the abscissa axis. The calculated energy amount is defined as triplet energy T₁.

$${T}_{1} [ eV ] = 1239.85 / {λ}_{edge} \qquad Conversion Equation ( F 1 )$$

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength region of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (produced by Hitachi High-Technologies Corporation) is usable. The measurement apparatus is not limited thereto. A combination of a cooling unit, a low temperature container, an excitation light source and a light-receiving unit may be used for measurement.

### Singlet Energy S₁

A method of measuring a singlet energy S₁ with use of a solution (occasionally referred to as a solution method) is exemplified by a method below.

A toluene solution of a measurement target compound at a concentration ranging from 10⁻⁵ mol/L to 10⁻⁴ mol/L is prepared and put in a quartz cell. An absorption spectrum (ordinate axis: absorption intensity, abscissa axis: wavelength) of the thus-obtained sample is measured at a normal temperature (300K). A tangent is drawn to the fall of the absorption spectrum close to the long-wavelength region, and a wavelength value λedge (nm) at an intersection of the tangent and the abscissa axis is assigned to a conversion equation (F2) below to calculate a singlet energy.

$${S}_{1} [ eV ] = 1239 \text{.85} / λ edge \qquad Conversion Equation ( F 2 )$$

Any apparatus for measuring the absorption spectrum is usable. For instance, a spectrophotometer (U3310 produced by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

In an exemplary arrangement of the organic EL device of the exemplary embodiment, the triplet energy of the first host material T₁(H1) satisfies a relationship of a numerical formula (Numerical Formula 12) below.

$${T}_{1} ( H 1 ) > 2. eV \qquad ( Numerical Formula 12 )$$

In an exemplary arrangement of the organic EL device of the exemplary embodiment, the triplet energy of the first host material T₁(H1) satisfies a relationship of a numerical formula (Numerical Formula 12A) or a numerical formula (Numerical Formula 12B) below.

$${T}_{1} ( H 1 ) > 2.1 eV \qquad ( Numerical Formula 12 A )$$

$${T}_{1} ( H 1 ) > 2.15 eV \qquad ( Numerical Formula 12 B )$$

In the organic EL device according to the exemplary embodiment, when the triplet energy of the first host material T₁(H1) satisfies the relationship of the numerical formula (Numerical Formula 12A) or the numerical formula (Numerical Formula 12B), triplet excitons generated in the first emitting layer easily transfer to the second emitting layer, and also are easily inhibited from back-transferring from the second emitting layer to the first emitting layer. Consequently, singlet excitons are efficiently generated in the second emitting layer, thereby improving luminous efficiency.

In an exemplary arrangement of the organic EL device of the exemplary embodiment, the triplet energy of the first host material T₁(H1) satisfies a relationship of a numerical formula (Numerical Formula 12C) or (Numerical Formula 12D) below.

$$2.08 eV > {T}_{1} ( H 1 ) > 1.87 eV \qquad ( Numerical Formula 12 C )$$

$$2.05 eV > {T}_{1} ( H 1 ) > 1.9 eV \qquad ( Numerical Formula 12 D )$$

In the organic EL device according to the exemplary embodiment, when the triplet energy of the first host material T₁(H1) satisfies the relationship of the numerical formula (Numerical Formula 12C) or the numerical formula (Numerical Formula 12D), the energy of triplet excitons generated in the first emitting layer is reduced. The organic EL device of the exemplary embodiment can thus be expected to have a long lifetime.

In an exemplary arrangement of the organic EL device of the exemplary embodiment, the triplet energy of the first luminescent compound T₁(D1) satisfies a relationship of a numerical formula (Numerical Formula 14), (Numerical Formula 14A), or (Numerical Formula 14B) below.

$$2.7 eV > {T}_{1} ( D 1 ) \qquad ( Numerical Formula 14 )$$

$$2.6 eV > {T}_{1} ( D 1 ) \qquad ( Numerical Formula 14 A )$$

$$2.5 eV > {T}_{1} ( D 1 ) \qquad ( Numerical Formula 14 B )$$

The organic EL device has a long lifetime when the first emitting layer contains the first luminescent compound that satisfies the relationship of the numerical formula (Numerical Formula 14), (Numerical Formula 14A), or (Numerical Formula 14B).

In an exemplary arrangement of the organic EL device of the exemplary embodiment, the first emitting layer contains 0.5 mass % or more of the first luminescent compound with respect to the total mass of the first emitting layer.

In an exemplary arrangement of the organic EL device of the exemplary embodiment, the first emitting layer contains the first luminescent compound at 10 mass % or less, 7 mass % or less, or 5 mass % or less with respect to the total mass of the first emitting layer.

In an exemplary arrangement of the organic EL device of the exemplary embodiment, the first emitting layer contains the first compound as the first host material at 60 mass % or more, 70 mass % or more, 80 mass % or more, 90 mass % or more, or 95 mass % or more with respect to the total mass of the first emitting layer.

In an exemplary arrangement of the organic EL device of the exemplary embodiment, the first emitting layer contains 99 mass % or less of the first host material with respect to the total mass of the first emitting layer.

When the first emitting layer contains the first host material and the first luminescent compound, the upper limit of the total of the content ratios of the first host material and the first luminescent compound is 100 mass %.

The first emitting layer of the exemplary embodiment may further contain any other material than the first host material and the first luminescent compound.

The first emitting layer may contain a single type of the first host material or may contain two or more types of the first host material. The first emitting layer may contain a single type of the first luminescent compound or may contain two or more types of the first luminescent compound.

In an exemplary arrangement of the organic EL device of the exemplary embodiment, the first emitting layer may only contain the first host material and the first luminescent compound.

In an exemplary arrangement of the organic EL device of the exemplary embodiment, the first emitting layer has a film thickness of 3 nm or more. A film thickness of 3 nm or more of the first emitting layer is sufficient for causing recombination of holes and electrons in the first emitting layer.

In an exemplary arrangement of the organic EL device of the exemplary embodiment, the first emitting layer has a film thickness of 15 nm or less. A film thickness of 15 nm or less of the first emitting layer is thin enough for transfer of triplet excitons to the second emitting layer.

In an exemplary arrangement of the organic EL device of the exemplary embodiment, the first emitting layer has a film thickness in a range from 3 to 15 nm.

### First Host Material

In an exemplary arrangement of the organic EL device of the exemplary embodiment, the first host material includes, in a molecule, at least one of a structure of Condition (i) below or a structure of Condition (ii) below.

Condition (i): a biphenyl structure including a first benzene ring and a second benzene ring linked to each other with a single bond, the first benzene ring and the second benzene ring in the biphenyl structure being further linked to each other by cross-linking at at least one site other than the single bond, and

Condition (ii): a first linking structure including a benzene ring and a naphthalene ring linked to each other with a single bond, the benzene ring and the naphthalene ring in the first linking structure being each independently further fused or not fused with a monocyclic ring or fused ring, the benzene ring and the naphthalene ring in the first linking structure being further linked to each other by cross-linking at at least one site other than the single bond.

In an exemplary arrangement of the organic EL device of the exemplary embodiment, the first host material includes, in a molecule, the structure of Condition (i) above.

In an exemplary arrangement of the organic EL device of the exemplary embodiment, the first benzene ring and the second benzene ring in the biphenyl structure of Condition (i) above are further linked to each other by the cross-linking of Condition (i) above at one site other than the single bond.

When the first host material has the biphenyl structure including such cross-linking, deterioration in chromaticity of the organic EL device that includes the emitting layer containing the first host material is expected to be inhibited.

In an exemplary arrangement of the organic EL device of the exemplary embodiment, the first benzene ring and the second benzene ring in the biphenyl structure of Condition (i) above are further linked to each other by the cross-linking of Condition (i) above at two sites other than the single bond.

In an exemplary arrangement of the organic EL device of the exemplary embodiment, the cross-linking of Condition (i) above includes a double bond.

In an exemplary arrangement of the organic EL device of the exemplary embodiment, the cross-linking of Condition (i) above includes no double bond.

In an exemplary arrangement of the organic EL device of the exemplary embodiment, the first host material includes, in a molecule, the structure of Condition (i) above, in which the first benzene ring and the second benzene ring in the biphenyl structure are further linked to each other by the cross-linking of Condition (i) above at two sites other than the single bond and the cross-linking of Condition (i) above includes no double bond. When the first host material has the biphenyl structure including such cross-linking, deterioration in chromaticity of the organic EL device that includes the emitting layer containing the first host material is expected to be inhibited.

For instance, assuming that the first benzene ring and the second benzene ring in the biphenyl structure represented by a formula (BP1) below are further linked to each other by cross-linking at at least one site other than the single bond, the biphenyl structure is exemplified by linking structures (fused rings) represented by formulae (BP11) to (BP15) below.

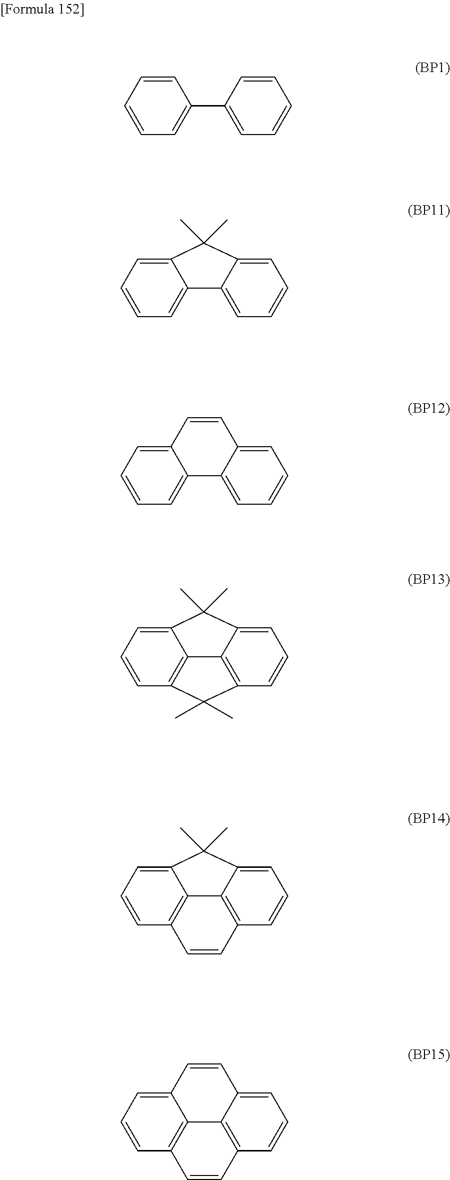

The formula (BP11) represents a linking structure in which the first benzene ring and the second benzene ring are linked to each other at one site other than the single bond by cross-linking including no double bond.

The formula (BP12) represents a linking structure in which the first benzene ring and the second benzene ring are linked to each other at one site other than the single bond by cross-linking including a double bond.

The formula (BP13) represents a linking structure in which the first benzene ring and the second benzene ring are linked to each other at two sites other than the single bond by cross-linking including no double bond.

The formula (BP14) represents a linking structure in which the first benzene ring and the second benzene ring are linked to each other by cross-linking including no double bond at one of two sites other than the single bond, and the first benzene ring and the second benzene ring are linked to each other by cross-linking including a double bond at the other of the two sites other than the single bond.

The formula (BP15) represents a linking structure in which the first benzene ring and the second benzene ring are linked to each other at two sites other than the single bond by cross-linking including a double bond.

In an exemplary arrangement of the organic EL device of the exemplary embodiment, the first host material includes, in a molecule, the structure of Condition (ii) above.

When the first host material has the linking structure including such cross-linking, deterioration in chromaticity of the organic EL device that includes the emitting layer containing the first host material is expected to be inhibited.

The first host material in the above case is only required to have a first linking structure as the minimum unit in a molecule, the first linking structure including a benzene ring and a naphthalene ring linked to each other with a single bond (occasionally referred to as a benzene-naphthalene linking structure), the first linking structure being as represented by a formula (X1) or a formula (X2) below. The benzene ring may be fused with a further monocyclic ring or fused ring, and the naphthalene ring may be fused with a further monocyclic ring or fused ring. For instance, also in a case where the first host material has, in a molecule, a second linking structure including a naphthalene ring and a naphthalene ring linked to each other with a single bond (occasionally referred to as a naphthalene-naphthalene linking structure), the second linking structure being as represented by a formula (X3), a formula (X4), or a formula (X5) below, the naphthalene-naphthalene linking structure is regarded as including the benzene-naphthalene linking structure since one of the naphthalene rings includes a benzene ring.

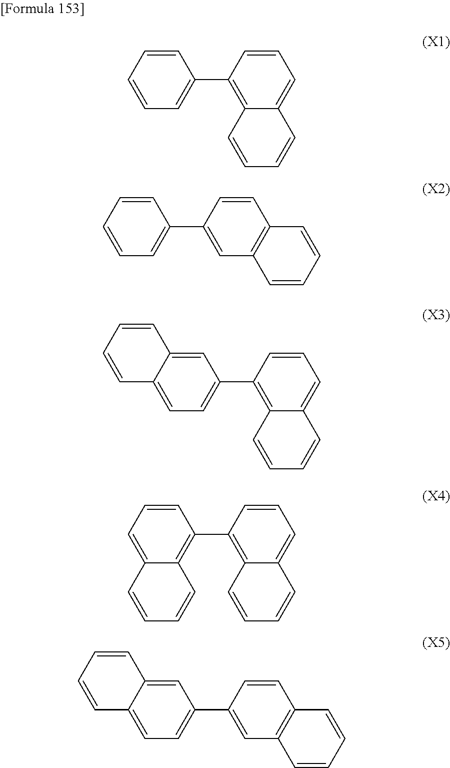

In an exemplary arrangement of the organic EL device of the exemplary embodiment, the cross-linking of Condition (ii) above includes a double bond. Specifically, the first host material of the exemplary embodiment also preferably has a structure in which the benzene ring and the naphthalene ring are further linked to each other at any other site than the single bond by the cross-linking structure including a double bond.

Assuming that the benzene ring and the naphthalene ring in the first linking structure (benzene-naphthalene linking structure) are further linked to each other at at least one site other than the single bond by cross-linking, for instance, a linking structure (fused ring) represented by a formula (X11) below is obtained in a case of the formula (X1), and a linking structure (fused ring) represented by a formula (X31) below is obtained in a case of the formula (X3).

Assuming that the benzene ring and the naphthalene ring in the benzene-naphthalene linking structure are further linked to each other at any other site than the single bond by cross-linking including a double bond, for instance, a linking structure (fused ring) represented by a formula (X12) below is obtained in a case of the formula (X1), a linking structure (fused ring) represented by a formula (X21), formula (X22), or formula (X23) below is obtained in a case of the formula (X2), a linking structure (fused ring) represented by a formula (X41) below is obtained in a case of the formula (X4), and a linking structure (fused ring) represented by a formula (X51) below is obtained in a case of the formula (X5).

Assuming that the benzene ring and the naphthalene ring in the benzene-naphthalene linking structure are further linked to each other at at least one site other than the single bond by cross-linking including a heteroatom (e.g., an oxygen atom), for instance, a linking structure (fused ring) represented by a formula (X13) below is obtained in a case of the formula (X1).

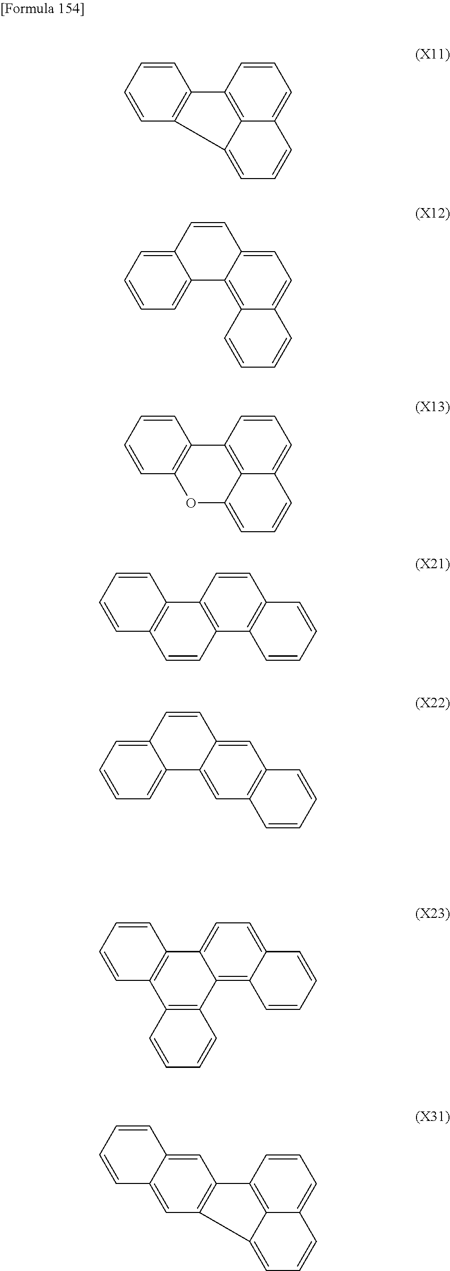

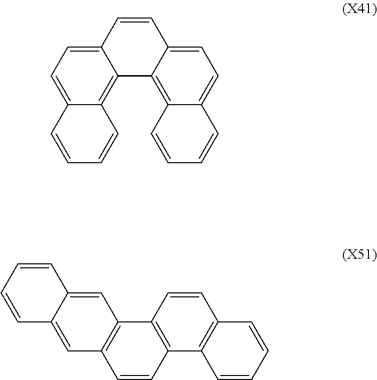

In an exemplary arrangement of the organic EL device of the exemplary embodiment, the first host material is a compound selected from the group consisting of a compound represented by a formula (H11), a compound represented by a formula (H12), a compound represented by a formula (H13), a compound represented by a formula (H14), a compound represented by a formula (H15), and a compound represented by a formula (H16) below.

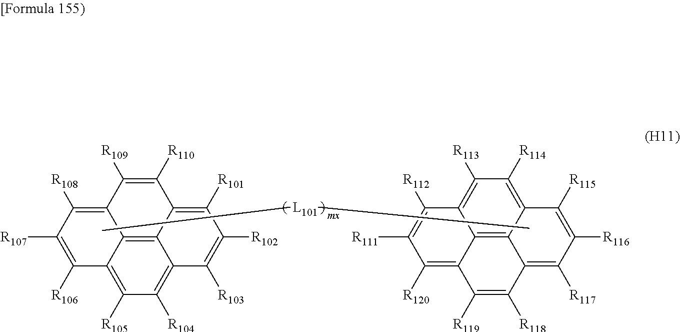

In the formula (H11):
R₁₀₁ to R₁₁₀ and R₁₁₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
one of R₁₀₁ to R₁₁₀ represents a bonding position to L₁₀₁, and one of R₁₁₁ to R₁₂₀ represents a bonding position to L₁₀₁;
L₁₀₁ is a single bond, a substituted or unsubstituted arylene group having 6 to 24 ring carbon atoms, or a substituted or unsubstituted divalent heterocyclic group having 5 to 24 ring atoms;
mx is 0, 1, 2, 3, 4, or 5; and
when two or more L₁₀₁ are present, the two or more L₁₀₁ are mutually the same or different.

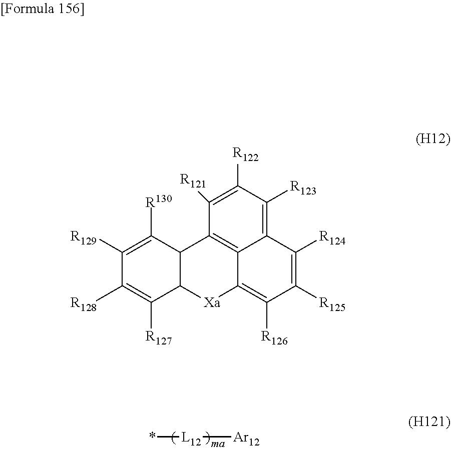

In the formula (H12):
Xa is an oxygen atom, a sulfur atom, C(R₁₂₀₁)(R₁₂₀₂), or Si(R₁₂₀₃)(R₁₂₀₄);
R₁₂₀₁ to R₁₂₀₄ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
at least one combination of adjacent two or more of R₁₂₁ to R₁₃₀ are mutually bonded to form a substituted or unsubstituted monocyclic ring, mutually bonded to form a substituted or unsubstituted fused ring, or not mutually bonded;
R₁₂₁ to R₁₃₀ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, a substituted or unsubstituted heterocyclic group having 5 to 50 ring atoms, or a group represented by a formula (H121) above;
at least one of R₁₂₁ to R₁₃₀ is a group represented by the formula (H121);
when a plurality of groups represented by the formula (H121) are present, the plurality of groups represented by the formula (H121) are mutually the same or different;
L₁₂ is a single bond, a substituted or unsubstituted arylene group having 6 to 50 ring carbon atoms, or a substituted or unsubstituted divalent heterocyclic group having 5 to 50 ring atoms;
ma is 0, 1, 2 or 3;
when two or more L₁₂ are present, the two or more L₁₂ are mutually the same or different;
Ar₁₂ is a substituted or unsubstituted aryl group having 6 to 50 ring carbon atoms, or a substituted or unsubstituted heterocyclic group having 5 to 50 ring atoms;
when two or more Ar₁₂ are present, the two or more Ar₁₂ are mutually the same or different; and
* in the formula (H121) represents a bonding position.

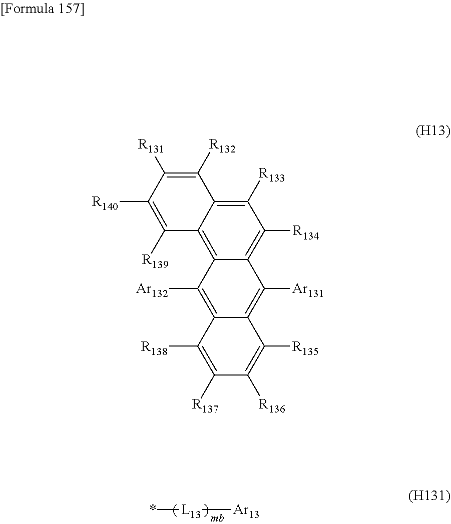

In the formula (H13):
at least one combination of adjacent two or more of R₁₃₁ to R₁₃₄ and R₁₃₉ to R₁₄₀ are mutually bonded to form a substituted or unsubstituted monocyclic ring, or not mutually bonded;
at least one combination of adjacent two or more of R₁₃₅ to R₁₃₈ are mutually bonded to form a substituted or unsubstituted monocyclic ring, or not mutually bonded;
Ar₁₃₁, Ar₁₃₂, and R₁₃₁ to R₁₄₀ not forming the substituted or unsubstituted monocyclic ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by a formula (H131) above;
at least one of R₁₃₁ to R₁₄₀, Ar₁₃₁, or Ar₁₃₂ is a group represented by the formula (H131);
when a plurality of groups represented by the formula (H131) are present, the plurality of groups represented by the formula (H131) are mutually the same or different;
L₁₃ is a single bond, a substituted or unsubstituted arylene group having 6 to 50 ring carbon atoms, or a substituted or unsubstituted divalent heterocyclic group having 5 to 50 ring atoms;
Ar₁₃ is a substituted or unsubstituted aryl group having 6 to 50 ring carbon atoms, or a substituted or unsubstituted heterocyclic group having 5 to 50 ring atoms; mb is 0, 1, 2, 3, 4, or 5;
when two or more L₁₃ are present, the two or more L₁₃ are mutually the same or different;
when two or more Ar₁₃ are present, the two or more Ar₁₃ are mutually the same or different; and
* in the formula (H131) represents a bonding position to a benz[a]anthracene ring in the formula (H13),

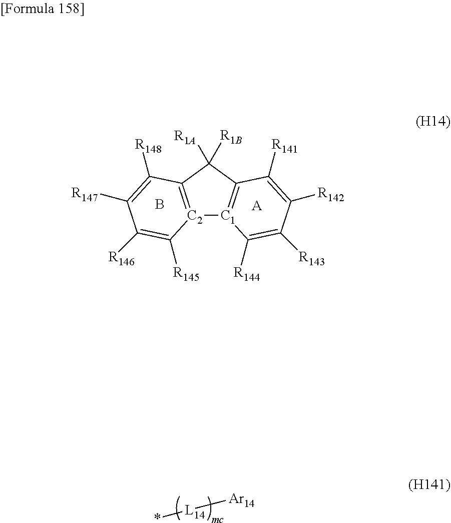

In the formula (H14):
R_{1A} and R_{1B} are each independently a substituted or unsubstituted alkyl group having 1 to 15 carbon atoms, a substituted or unsubstituted aryl group having 6 to 17 ring carbon atoms, or a substituted or unsubstituted heterocyclic group having 5 to 17 ring atoms;
at least one of R_{1A} or R_{1B} is a substituted or unsubstituted alkyl group having 1 to 15 carbon atoms;
a combination of adjacent two or more of R₁₄₁ to R₁₄₄ or a combination of adjacent two or more of R₁₄₅ to R₁₄₈ are mutually bonded to form a substituted or unsubstituted monocyclic ring, or mutually bonded to form a substituted or unsubstituted fused ring;
when a substituted or unsubstituted monocyclic ring or a substituted or unsubstituted fused ring is formed with a ring A, a group represented by a formula (H141) above is bonded to a carbon atom bonded to R₁₄₂ or, of carbon atoms forming the monocyclic ring with the ring A and the fused ring with the ring A, a carbon atom farthest from a carbon atom C₁ of the ring A, the carbon atom C₁ being bonded with a single bond to a carbon atom C₂ of a ring B;
when a substituted or unsubstituted monocyclic ring or a substituted or unsubstituted fused ring is formed with not the ring A but the ring B, a group represented by the formula (H141) is bonded to a carbon atom bonded to R₁₄₂; and
R₁₄₂ not being the group represented by the formula (H141), and R₁₄₁, R₁₄₃, R₁₄₄, and R₁₄₅ to R₁₄₈ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 17 ring carbon atoms, or a substituted or unsubstituted heterocyclic group having 5 to 17 ring atoms;
in the formula (H141):
Ar₁₄ is a substituted or unsubstituted aryl group having four or more fused rings or a substituted or unsubstituted heterocyclic group having four or more fused rings;
L₁₄ is a single bond, a substituted or unsubstituted arylene group having 6 to 17 ring carbon atoms, or a substituted or unsubstituted divalent heterocyclic group having 5 to 17 ring atoms;
mc is 0, 1, or 2;
* represents a bonding position to an atom forming a ring of the formula (H14); and
the compound represented by the formula (H14) does not have, in a molecule, three or more groups of a substituted or unsubstituted aryl group having four or more fused rings and a substituted or unsubstituted heterocyclic group having four or more fused rings.

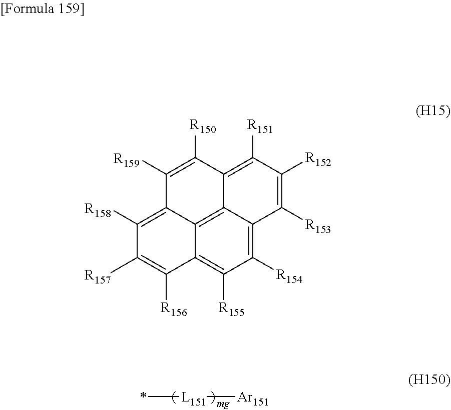

In the formula (H15):
R₁₅₀ to R₁₅₉ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by a formula (H150) above;
at least one of R₁₅₀ to R₁₅₉ is a group represented by the formula (H150);
when a plurality of groups represented by the formula (H150) are present, the plurality of groups represented by the formula (H150) are mutually the same or different;
L₁₅₁ is a single bond, a substituted or unsubstituted arylene group having 6 to 50 ring carbon atoms, or a substituted or unsubstituted divalent heterocyclic group having 5 to 50 ring atoms;
Ar₁₅₁ is a substituted or unsubstituted aryl group having 6 to 50 ring carbon atoms, or a substituted or unsubstituted heterocyclic group having 5 to 50 ring atoms; mg is 0, 1, 2, 3, 4, or 5;
when two or more L₁₅₁ are present, the two or more L₁₅₁ are mutually the same or different;
when two or more Ar₁₅₁ are present, the two or more Ar₁₅₁ are mutually the same or different; and
* in the formula (H150) represents a bonding position to a pyrene ring in the formula (H15).

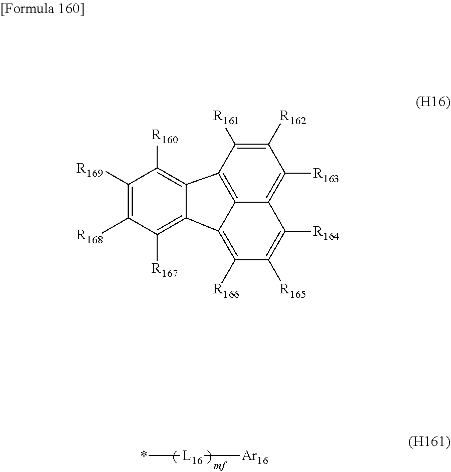

In the formula (H16):
at least one combination of adjacent two or more of R₁₆₀ to R₁₆₉ are mutually bonded to form a substituted or unsubstituted monocyclic ring, mutually bonded to form a substituted or unsubstituted fused ring, or not mutually bonded;
R₁₆₀ to R₁₆₉ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by a formula (H161) above;
at least one of a substituent, if present, for the substituted or unsubstituted monocyclic ring, a substituent, if present, for the substituted or unsubstituted fused ring, or R₁₆₀ to R₁₆₉ is a group represented by the formula (H161);
when a plurality of groups represented by the formula (H161) are present, the plurality of groups represented by the formula (H161) are mutually the same or different;
L₁₆ is a single bond, a substituted or unsubstituted arylene group having 6 to 50 ring carbon atoms, or a substituted or unsubstituted divalent heterocyclic group having 5 to 50 ring atoms;
Ar₁₆ is a substituted or unsubstituted aryl group having 6 to 50 ring carbon atoms, or a substituted or unsubstituted heterocyclic group having 5 to 50 ring atoms;
mf is 0, 1, 2, 3, 4, or 5;
when two or more L₁₆ are present, the two or more L₁₆ are mutually the same or different;
when two or more Ar₁₆ are present, the two or more Ar₁₆ are mutually the same or different; and
* in the formula (H161) represents a bonding position to a ring represented by the formula (H16).

In the first host material, R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In an exemplary arrangement of the organic EL device of the exemplary embodiment, the first host material is a compound selected from the group consisting of a compound represented by a formula (H111), a compound represented by a formula (H122), a compound represented by a formula (H132), and a compound represented by a formula (H133) below.

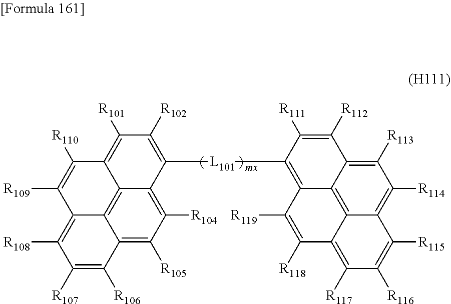

In the formula (H-111):

R₁₀₁, R₁₀₂, R₁₀₄ to R₁₁₀, and R₁₁₁ to R₁₁₉ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
L₁₀₁ and mx respectively represent the same as L₁₀₁ and mx in the formula (H11).

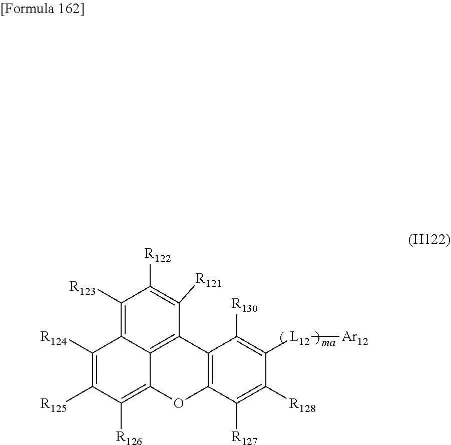

In the formula (H122):
R₁₂₁ to R₁₂₈ and R₁₃₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, or a substituted or unsubstituted heterocyclic group having 5 to 50 ring atoms; and
Ar₁₂, L₁₂, and ma respectively represent the same as Ar₁₂, L₁₂, and ma in the formula (H121).

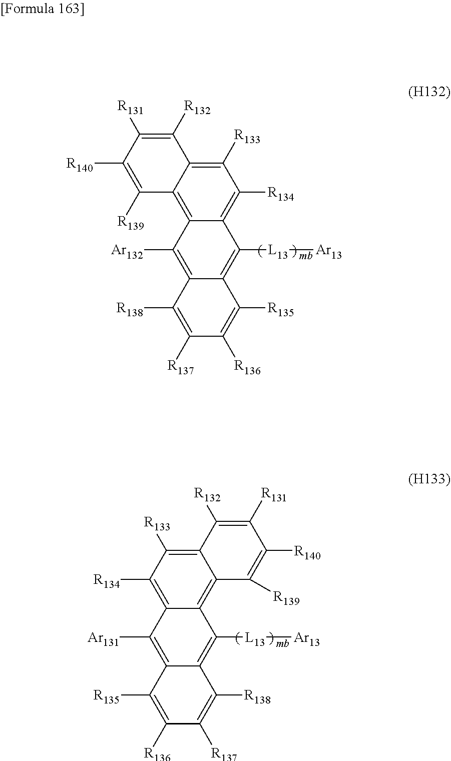

In the formulae (H132) and (H133):
R₁₃₁ to R₁₄₀, Ar₁₃₁, and Ar₁₃₂ are each a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
L₁₃, Ar₁₃, and mb respectively represent the same as L₁₃, Ar₁₃, and mb in the formula (H131).

In an exemplary arrangement of the organic EL device of the exemplary embodiment, the first host material is a compound having, in a molecule, at least one group represented by a formula (HX1) below.

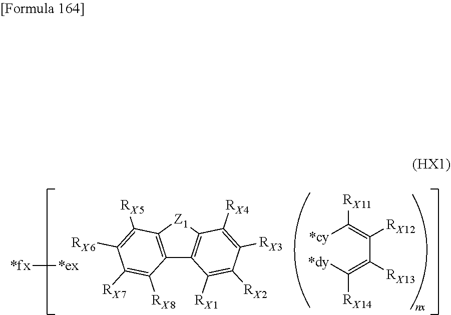

In the formula (HX1):
R_{X1} to R_{X8} and R_{X11} to R_{X14} are each independently a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms;
R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different;
when two or more R₉₀₇ are present, the two or more R₉₀₇ are mutually the same or different;
nx is 0 or 1;
when nx is 0, one selected from R_{X1} to R_{X8} is a single bond with *ex;
when nx is 1, one of R_{X1} and R_{X2}, one of R_{X2} and R_{X3}, or one of R_{X3} and R_{X5} is a single bond with *cx; the other of R_{X1} and R_{X2}, the other of R_{X2} and R_{X3}, or the other of R_{X3} and R_{X5} is a single bond with *dx; and one selected from R_{X5} to R_{X5}, R_{X11} to R_{X14}, and R_{X1} to R_{X4} being neither the single bond with *cx nor the single bond with *dx is a single bond with *ex;
Z₁ is an oxygen atom or a sulfur atom; and
*fx represents a bonding position to an atom in the first host material.

In an exemplary arrangement of the organic EL device of the exemplary embodiment, when nx in the formula (HX1) is zero, the group represented by the formula (HX1) is represented by a formula (HX10) below.

In an exemplary arrangement of the organic EL device of the exemplary embodiment, the first host material is a compound having, in a molecule, at least one group represented by the formula (HX10) below.

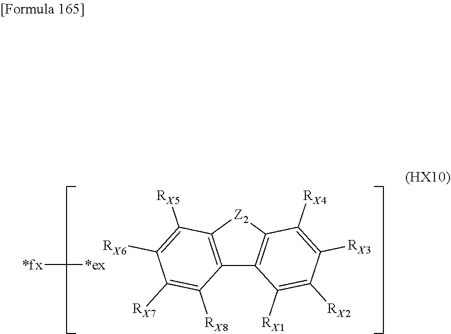

In the formula (HX10):
R_{X1} to R_{X8}, and Z₁ respectively represent the same as R_{X1} to R_{X8}, and Z₁ in the formula (HX1);
one selected from R_{X1} to R_{X8} is a single bond with *ex; and
*fx represents a bonding position to an atom in the first host material.

In an exemplary arrangement of the organic EL device of the exemplary embodiment, the first host material is a compound having, in a molecule, at least one group represented by the formula (HX1), in which nx is 1.

In an exemplary arrangement of the organic EL device of the exemplary embodiment, the first host material is a compound having, in a molecule, at least one group selected from the group consisting of groups represented by formulae (HX11), (HX12), and (HX13) below.

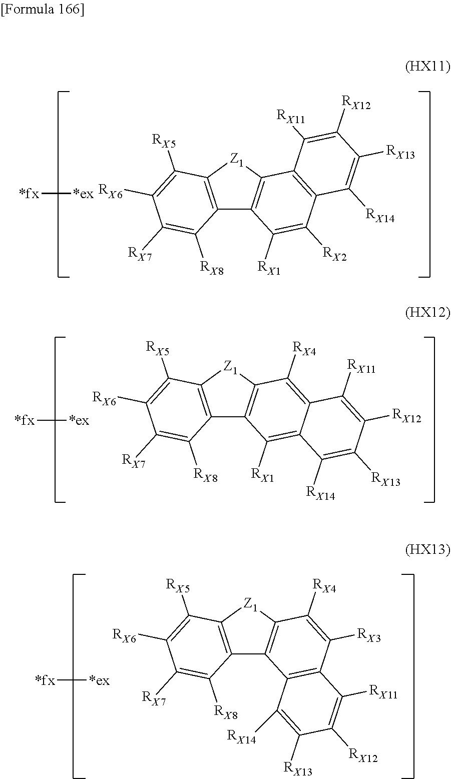

In the formulae (HX11), (HX12), and (HX13):
R_{X1} to R_{X8}, R_{X11} to R_{X14}, and Z₁ respectively represent the same as R_{X1} to R_{X8}, R_{X11} to R_{X14}, and Z₁ in the formula (HX1);
one of R_{X1} to R_{X8} and R_{X11} to R_{X14} in the formulae (HX11), (HX12), and (HX13) is a single bond with *ex; and
*fx represents a bonding position to an atom in the first host material.

In an exemplary arrangement of the organic EL device of the exemplary embodiment, the first host material is a compound represented by the formula (H13).

The compound represented by the formula (H13) has a benz[a]anthracene structure, and the ionization potential and affinity thereof are deep. Using the compound represented by the formula (H13) as the first host material increases the recombination probability of carriers in the first emitting layer, facilitating the luminous efficiency of the organic EL device. The organic EL device, in which the first emitting layer contains the compound represented by the formula (H13) as the first host material and at least one organic layer in the hole transporting zone contains the third compound represented by the formula (EB1), has an improved lifetime.

In an exemplary arrangement of the organic EL device of the exemplary embodiment, Ar₁₃ in the compound represented by the formula (H13) is a group selected from the group consisting of groups represented by the formulae (HX11), (HX12), and (HX13).

In an exemplary arrangement of the organic EL device of the exemplary embodiment, Ar₁₃ in the compound represented by the formula (H132) and the compound represented by the formula (H133) is a group selected from the group consisting of groups represented by the formulae (HX11), (HX12), and (HX13).

In an exemplary arrangement of the organic EL device of the exemplary embodiment, the hole injectability for the first host material (more specifically, for an aryl group in a molecule of the first host material) is improved when the first host material has, in a molecule, at least one group selected from the group consisting of groups represented by the formulae (HX1), (HX10), (HX11), (HX12), and (HX13). The hole injectability is more likely to be improved when the first host material has the benz[a]anthracene structure, as in the compound represented by the formula (H13).

In an exemplary arrangement of the organic EL device of the exemplary embodiment, the group represented by the formula (H150) is a group represented by a formula (H151) below.

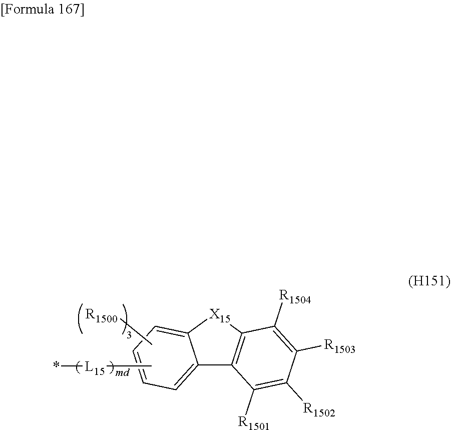

In the formula (H151):
X₁₅ is an oxygen atom or a sulfur atom;
L₁₅ is a single bond, a substituted or unsubstituted arylene group having 6 to 50 ring carbon atoms, or a substituted or unsubstituted divalent heterocyclic group having 5 to 50 ring atoms;
md is 0, 1, 2, 3, 4, or 5;
when two or more L₁₅ are present, the two or more L₁₅ are mutually the same or different;
at least one combination of adjacent two or more of R₁₅₀₀ to R₁₅₀₄ are mutually bonded to form a substituted or unsubstituted monocyclic ring, mutually bonded to form a substituted or unsubstituted fused ring, or not mutually bonded;
R₁₅₀₀ to R₁₅₀₄ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₆), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
a plurality of R₁₅₀₀ are mutually the same or different; and
* in the formula (H151) represents a bonding position to the pyrene ring in the formula (H15).

In an exemplary arrangement of the organic EL device of the exemplary embodiment, the first host material includes, in a molecule, neither a biscarbazole structure nor an amine structure.

In an exemplary arrangement of the organic EL device of the exemplary embodiment, the groups specified to be “substituted or unsubstituted” in the first host material are each an unsubstituted group.

### Method for Producing First Host Material

The first host material can be produced by a known method. The first host material can also be produced based on a known method through a known alternative reaction using a known material(s) tailored for the target compound.

### Specific Examples of First Host Material

Specific examples of the first host material include compounds below. The invention, however, is not limited to the specific examples of the first host material.

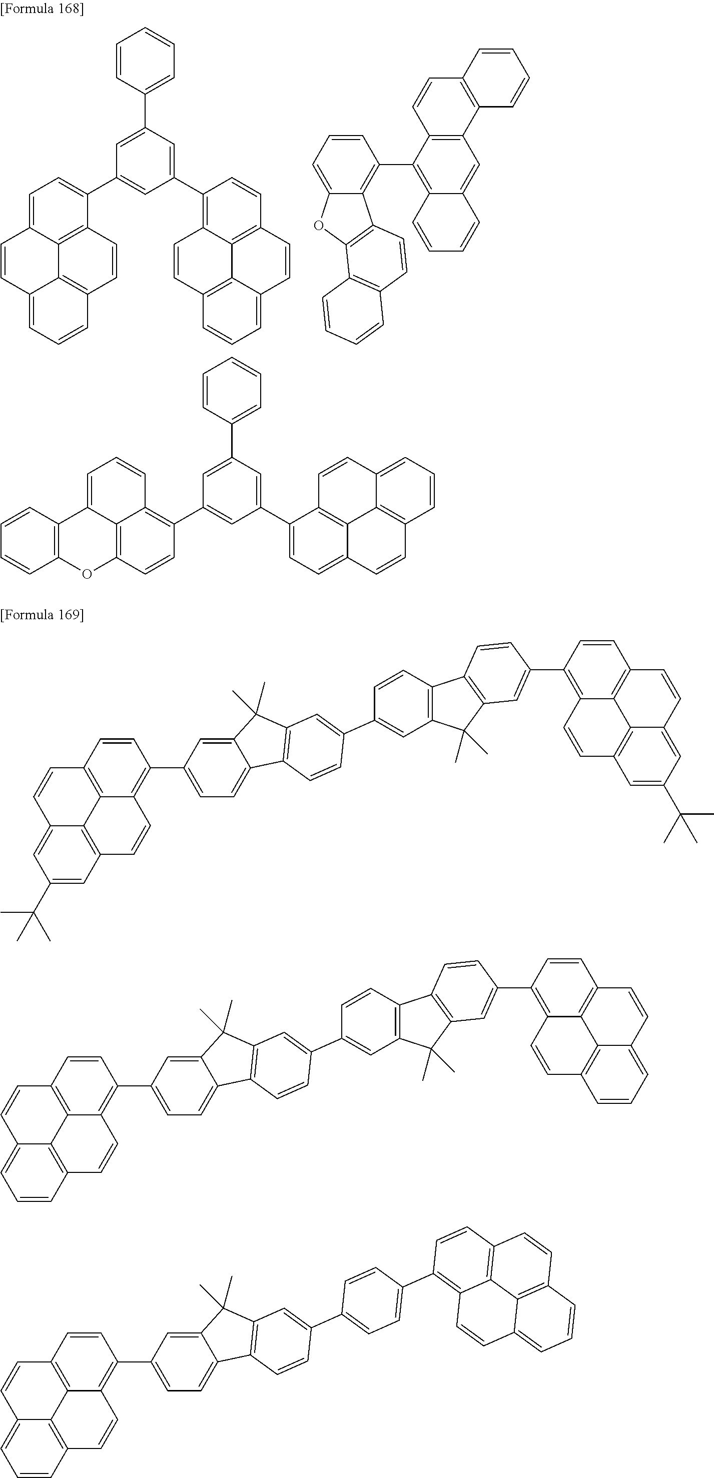

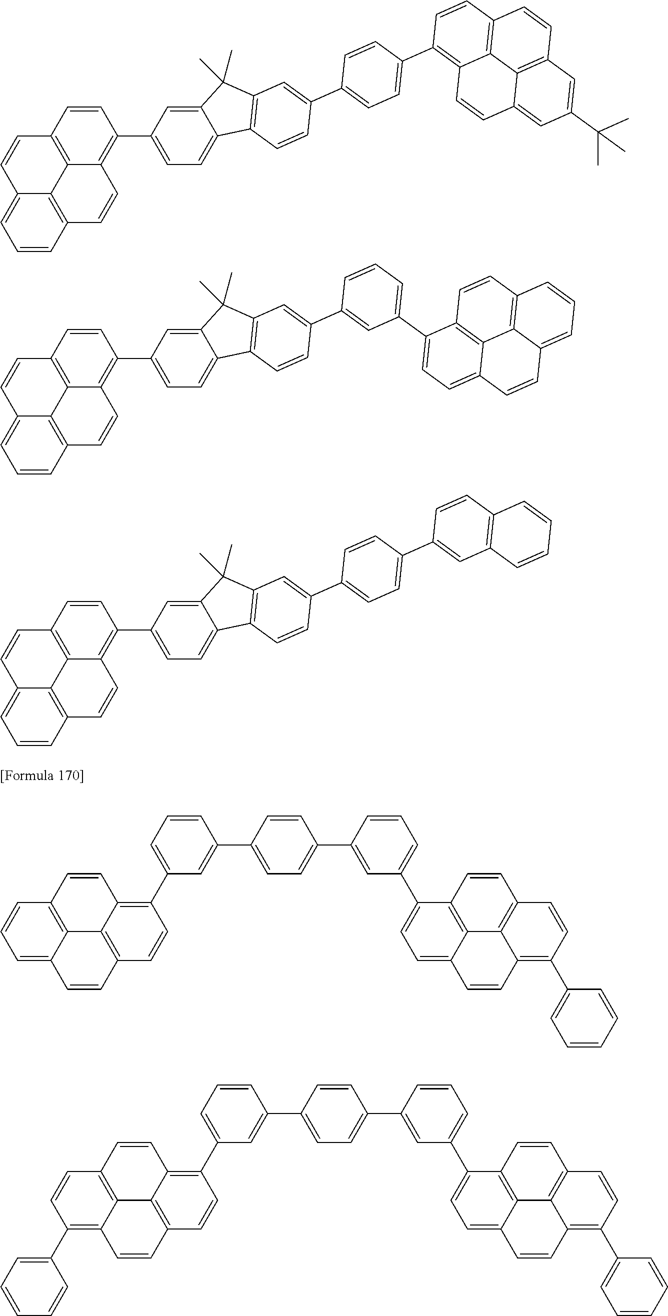

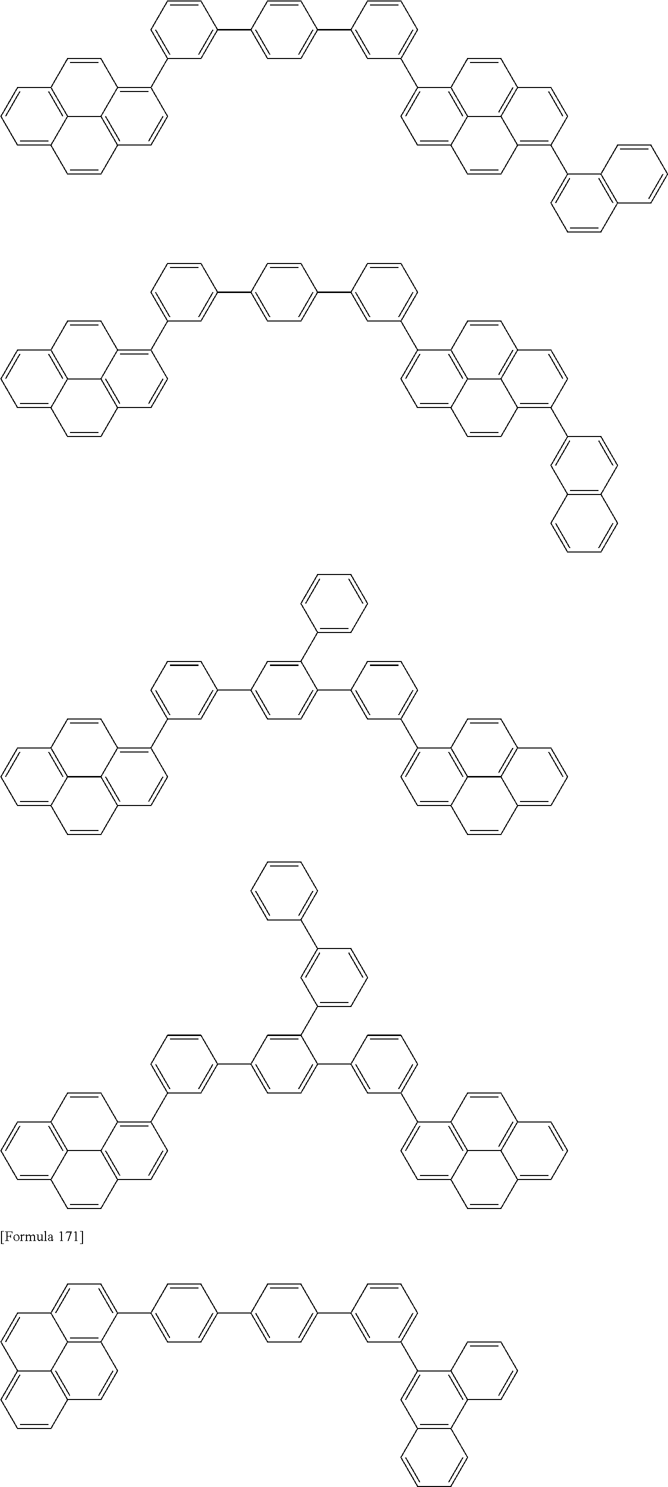

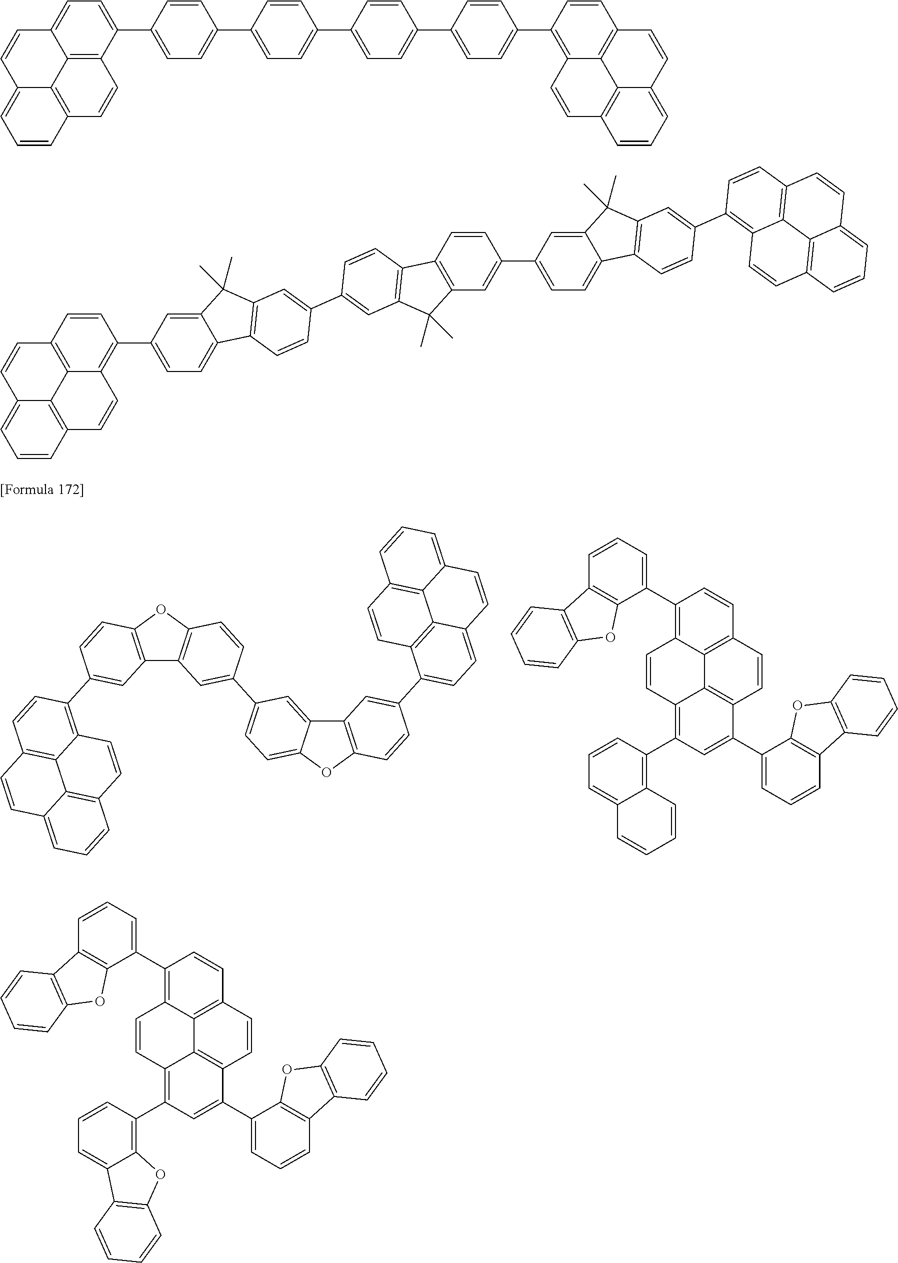

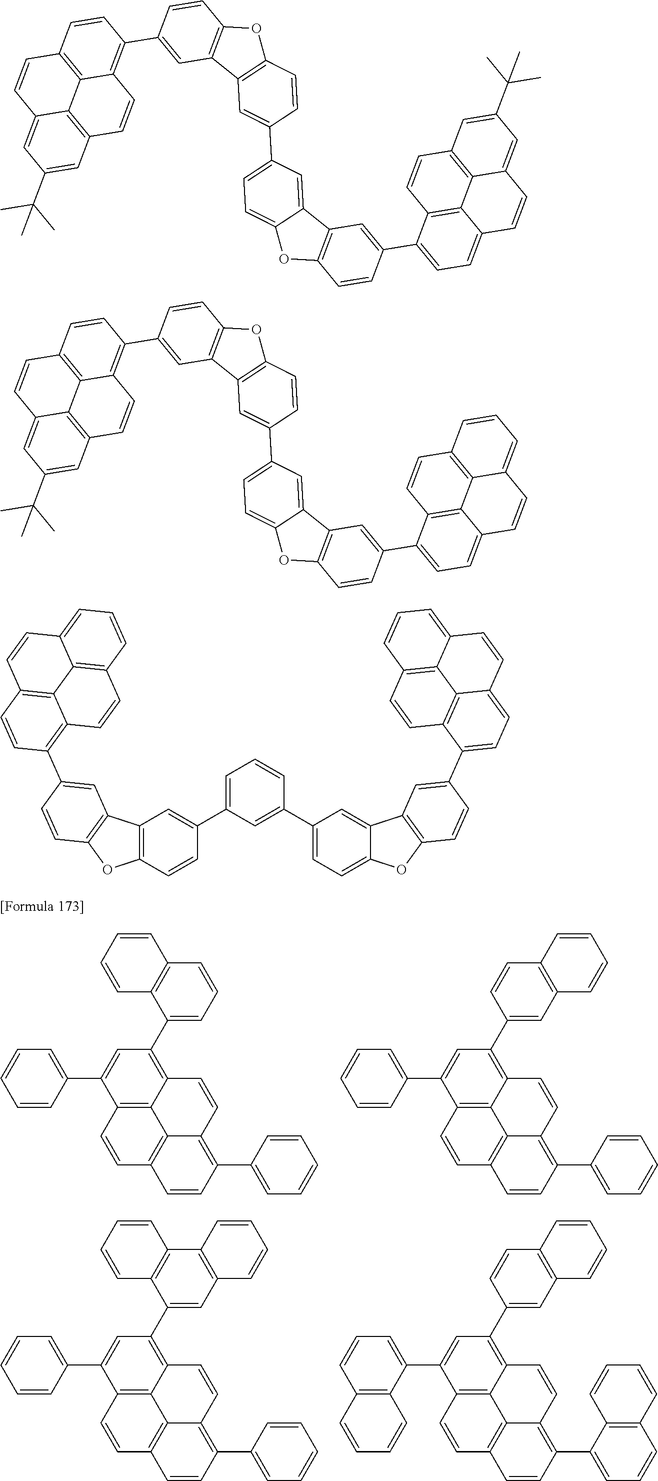

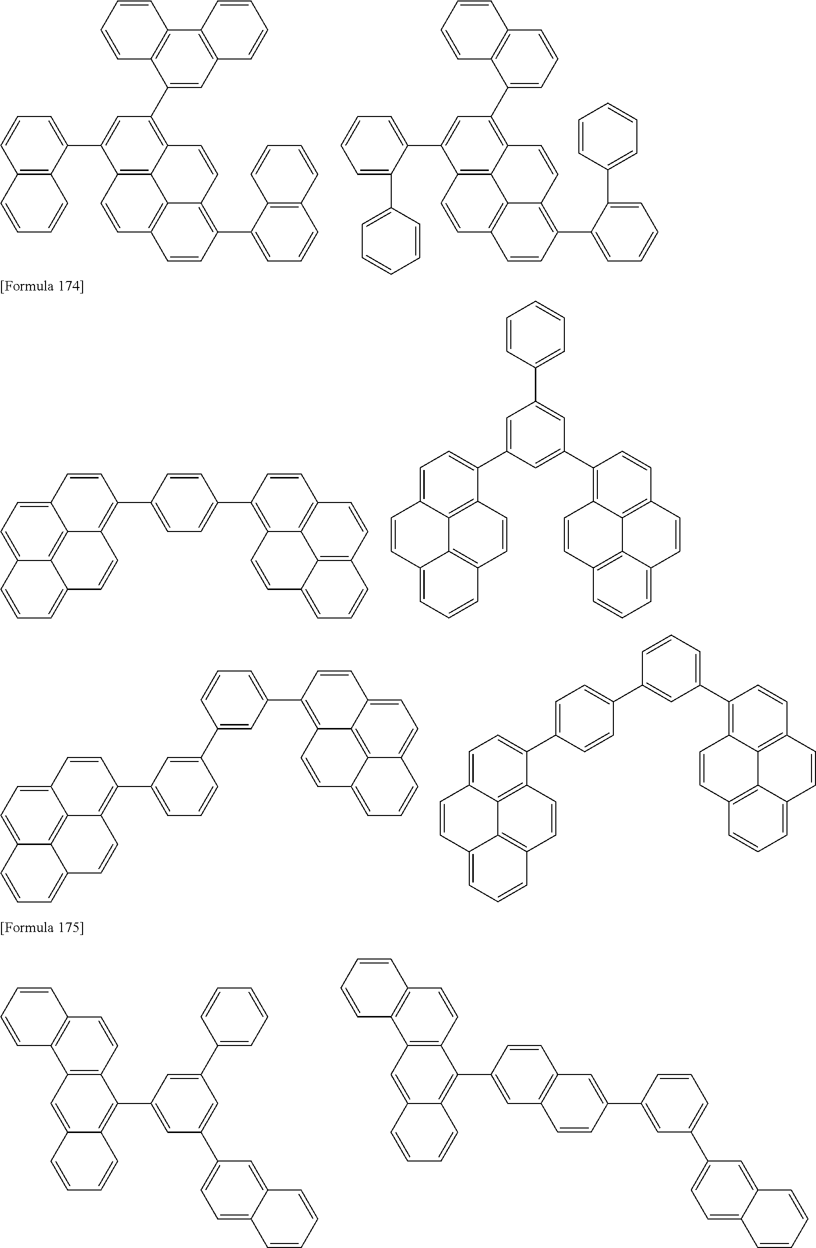

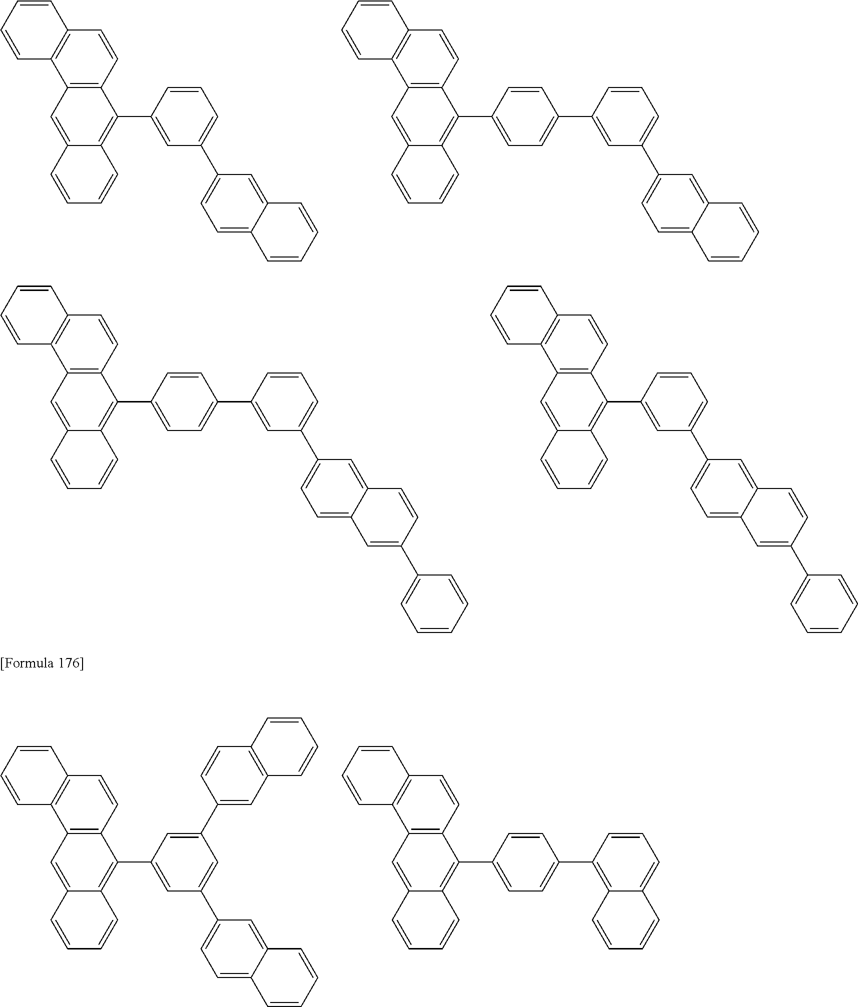

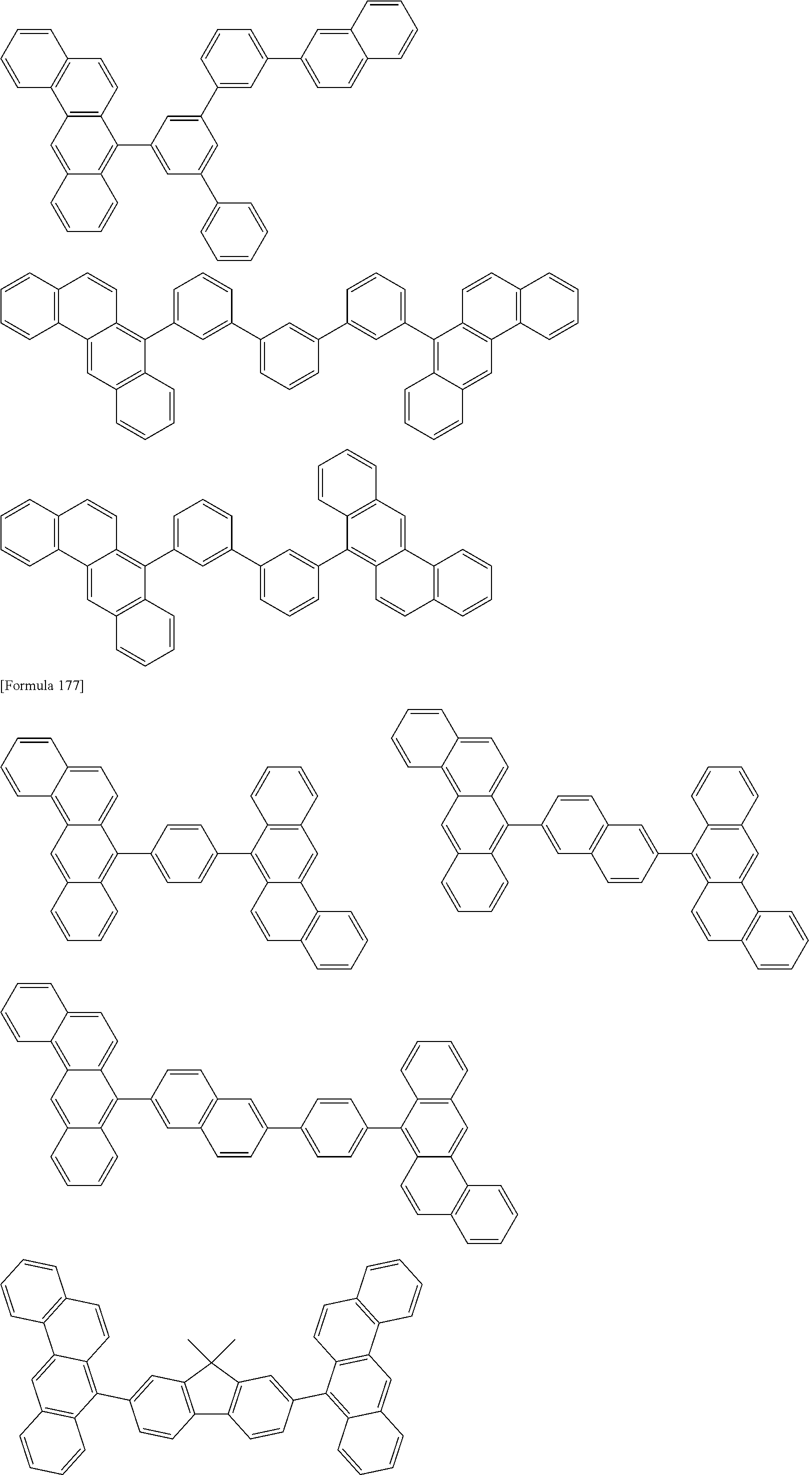

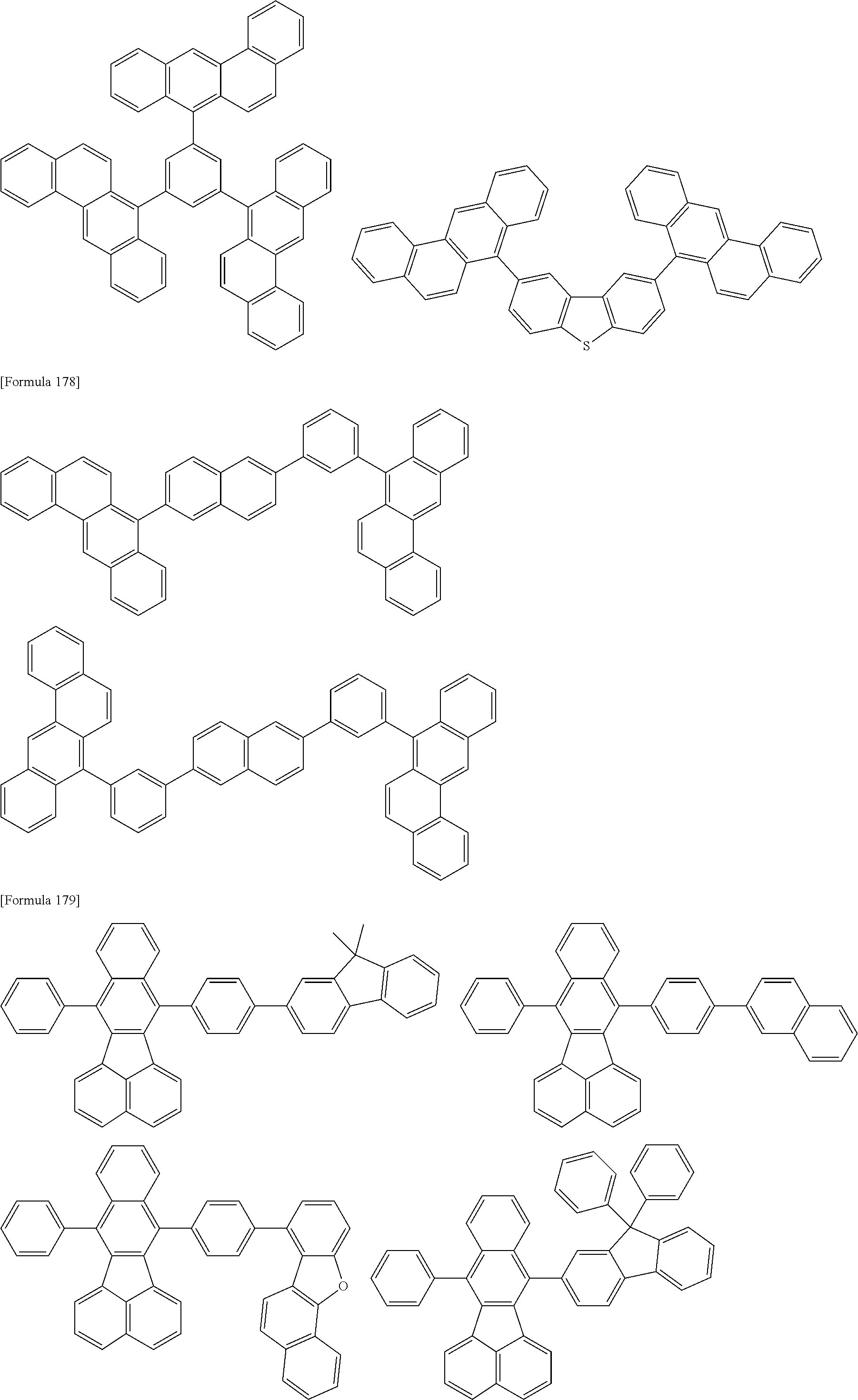

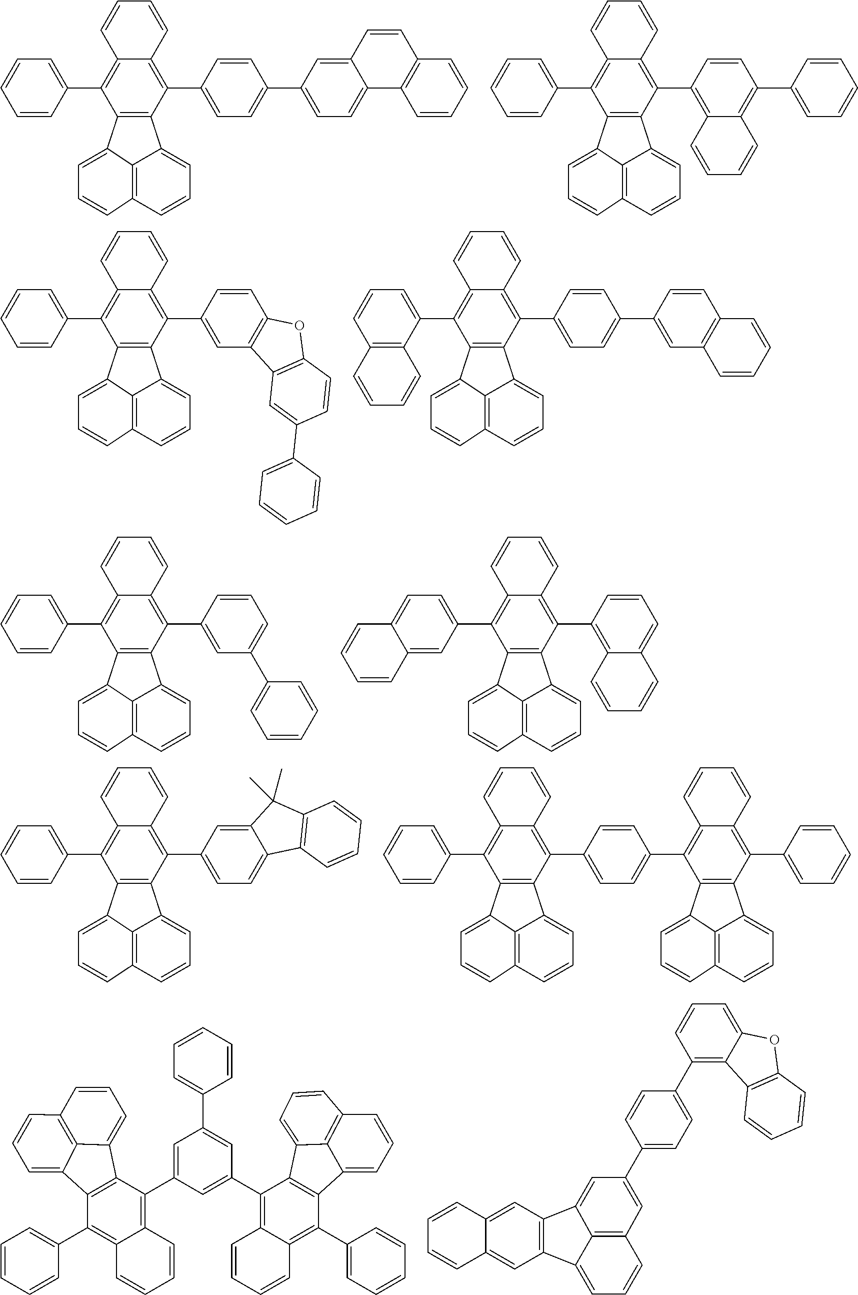

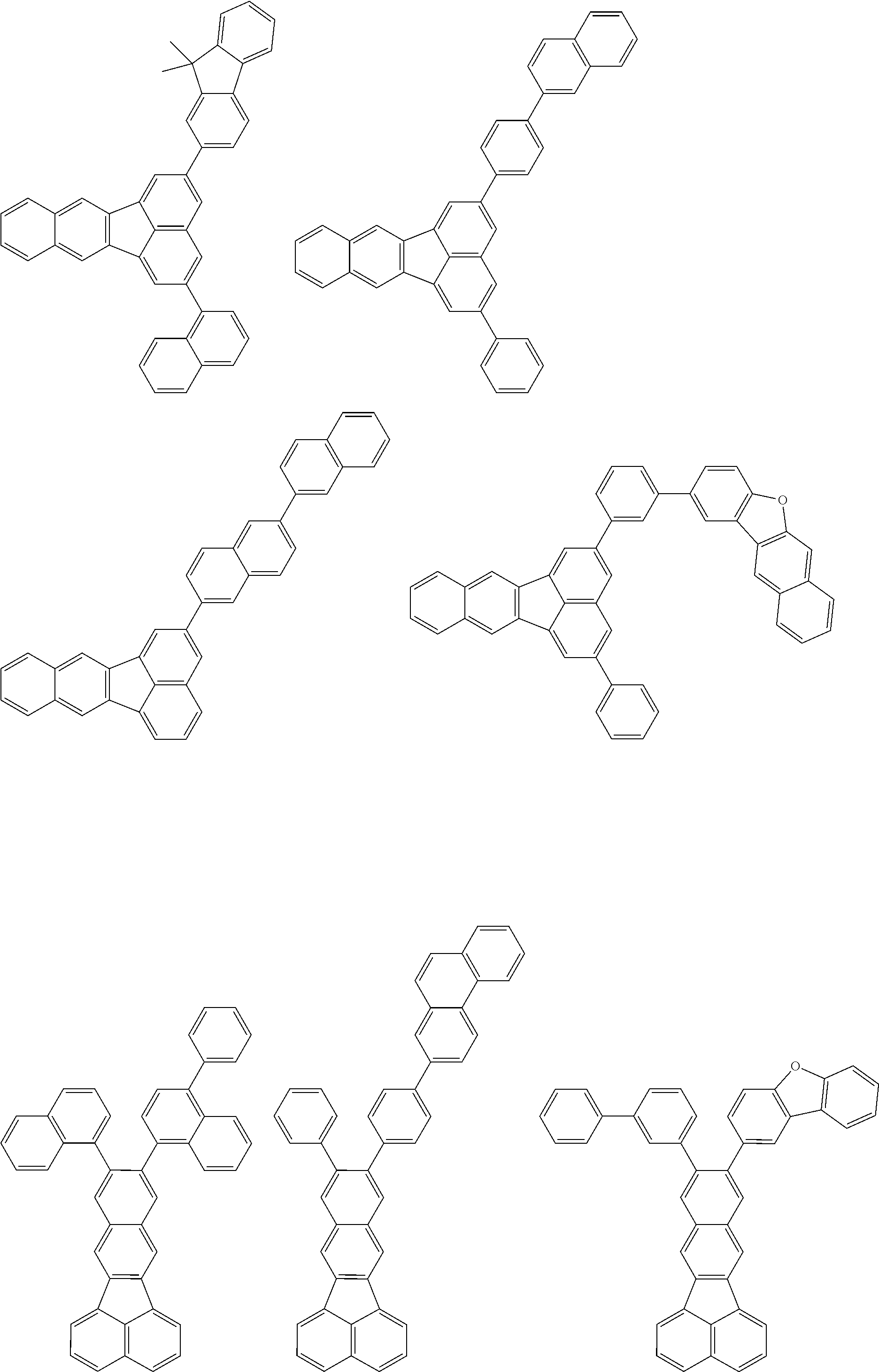

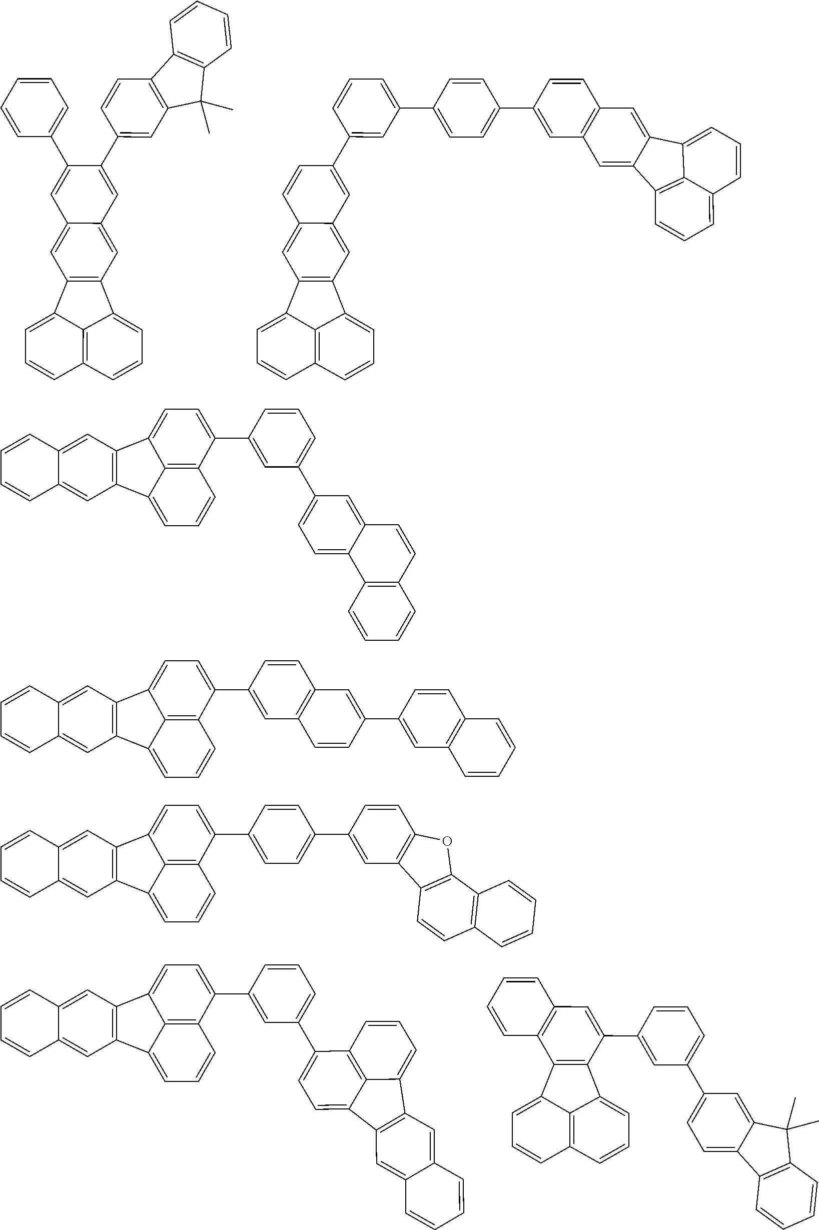

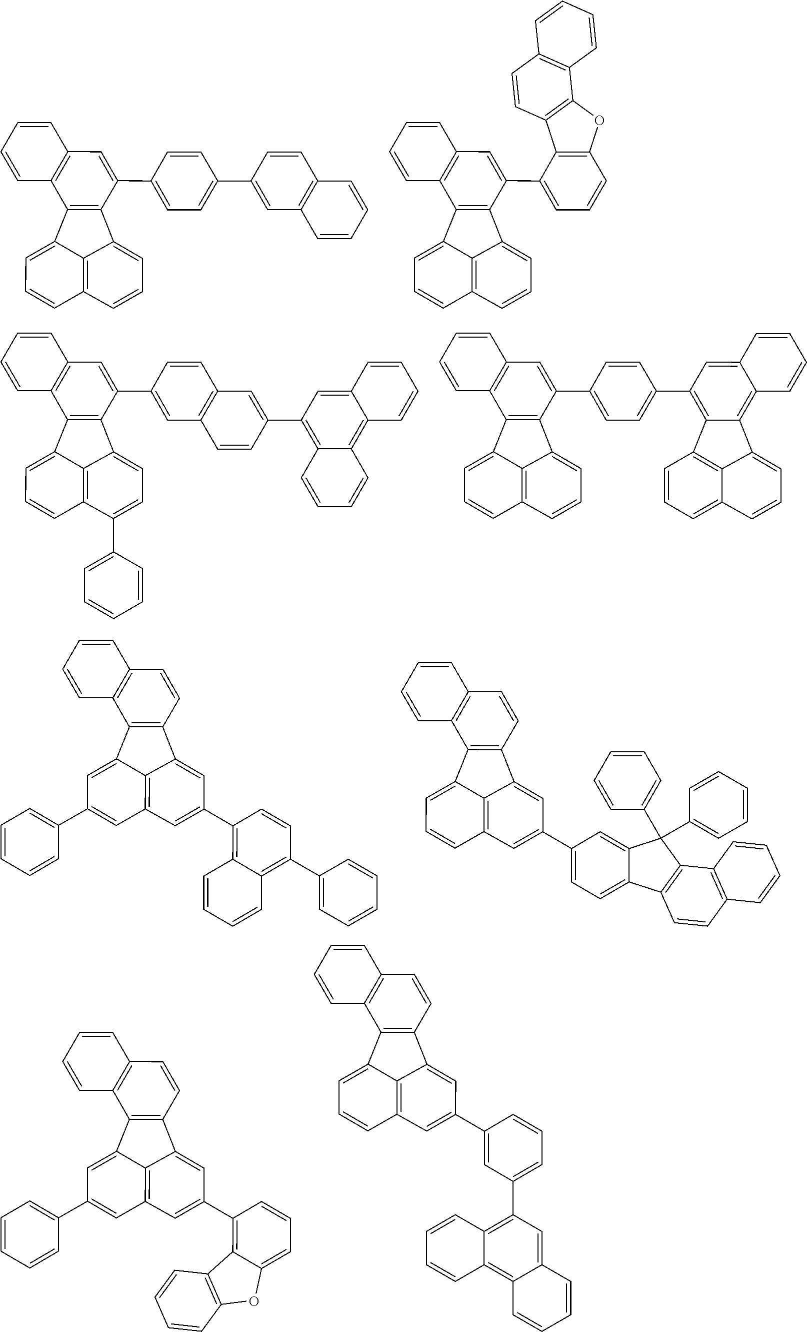

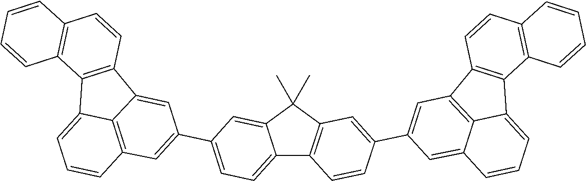 [SMILES: CC1(C)c2cc(-c3cc4c5c(cccc5c3)-c3c-4ccc4ccccc34)ccc2-c2ccc(-c3cc4c5c(cccc5c3)-c3c-4ccc4ccccc34)cc21]

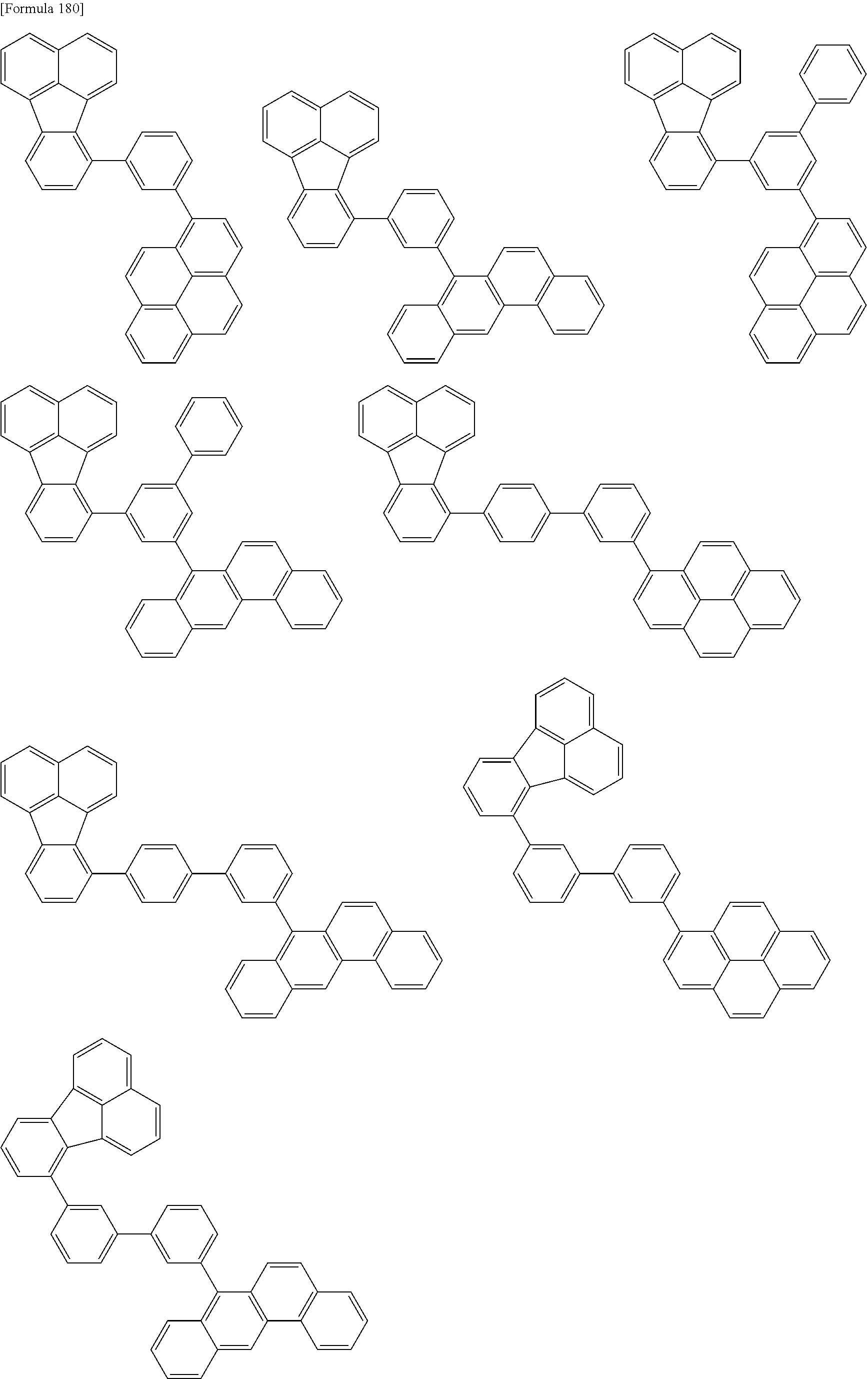

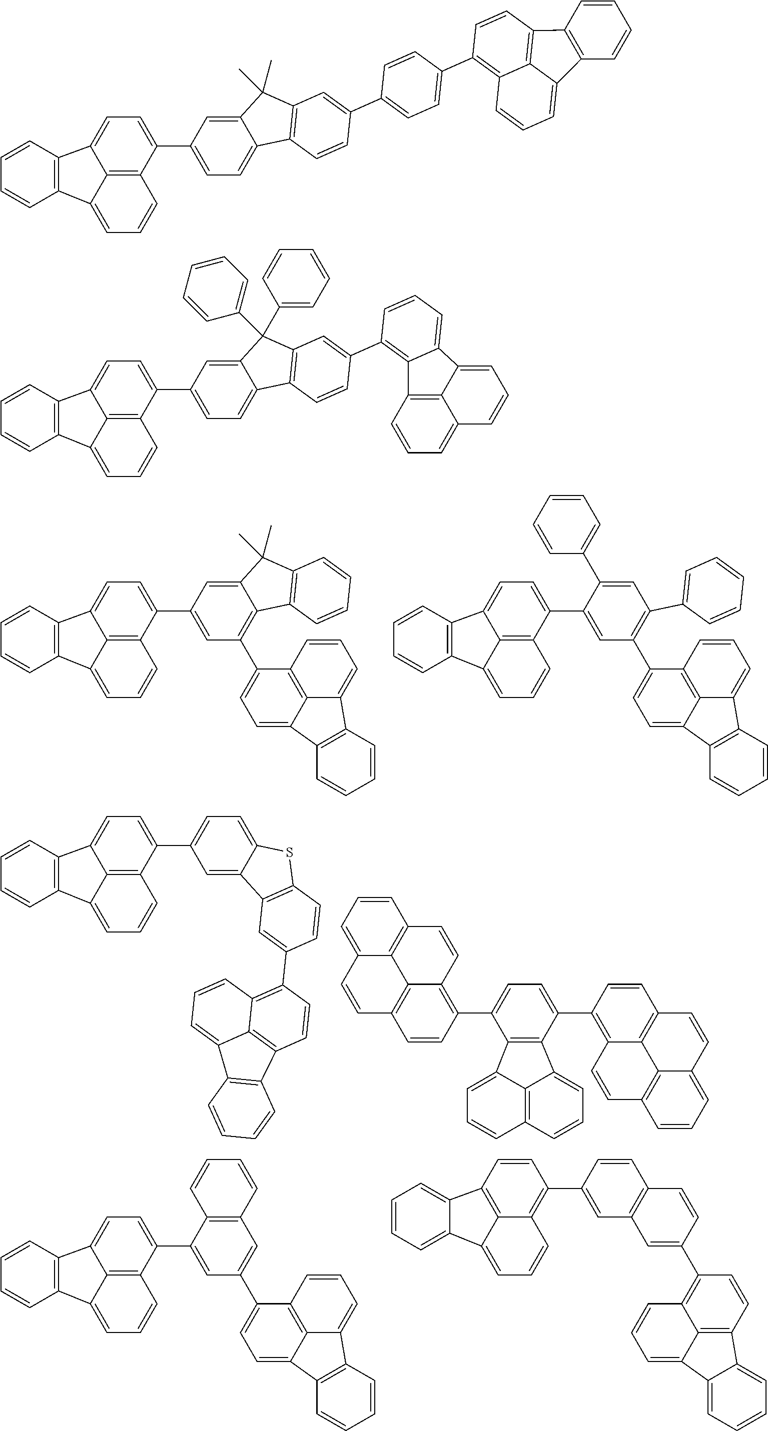

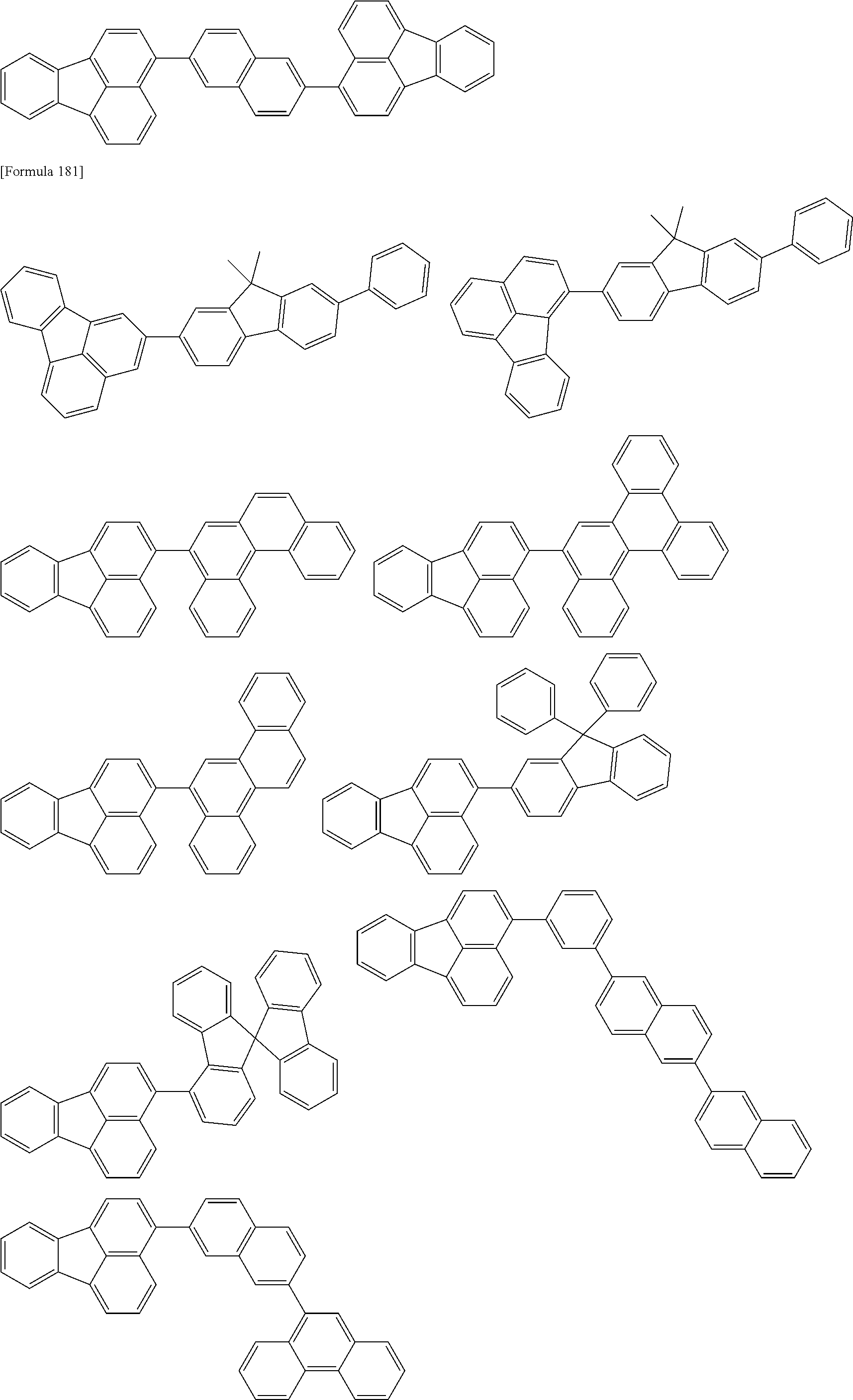

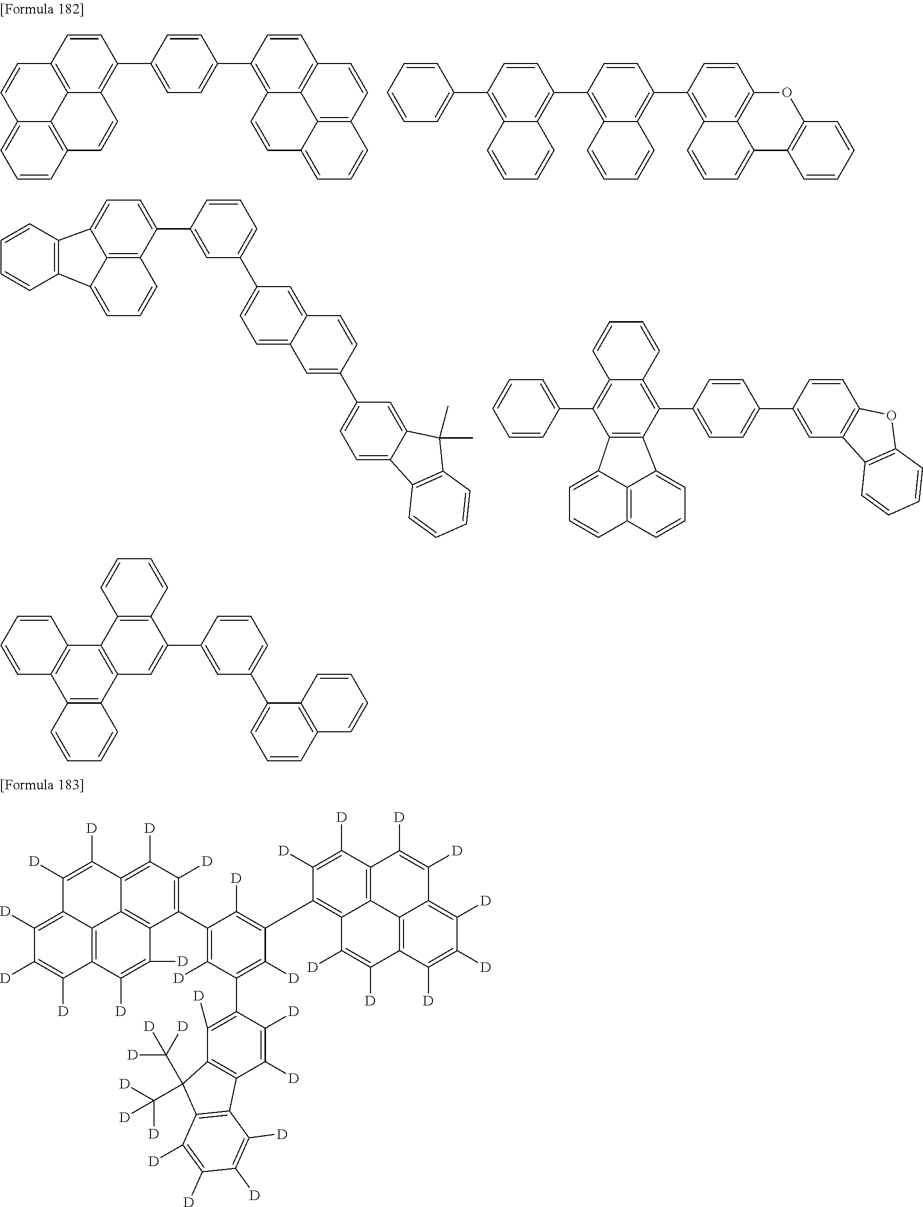

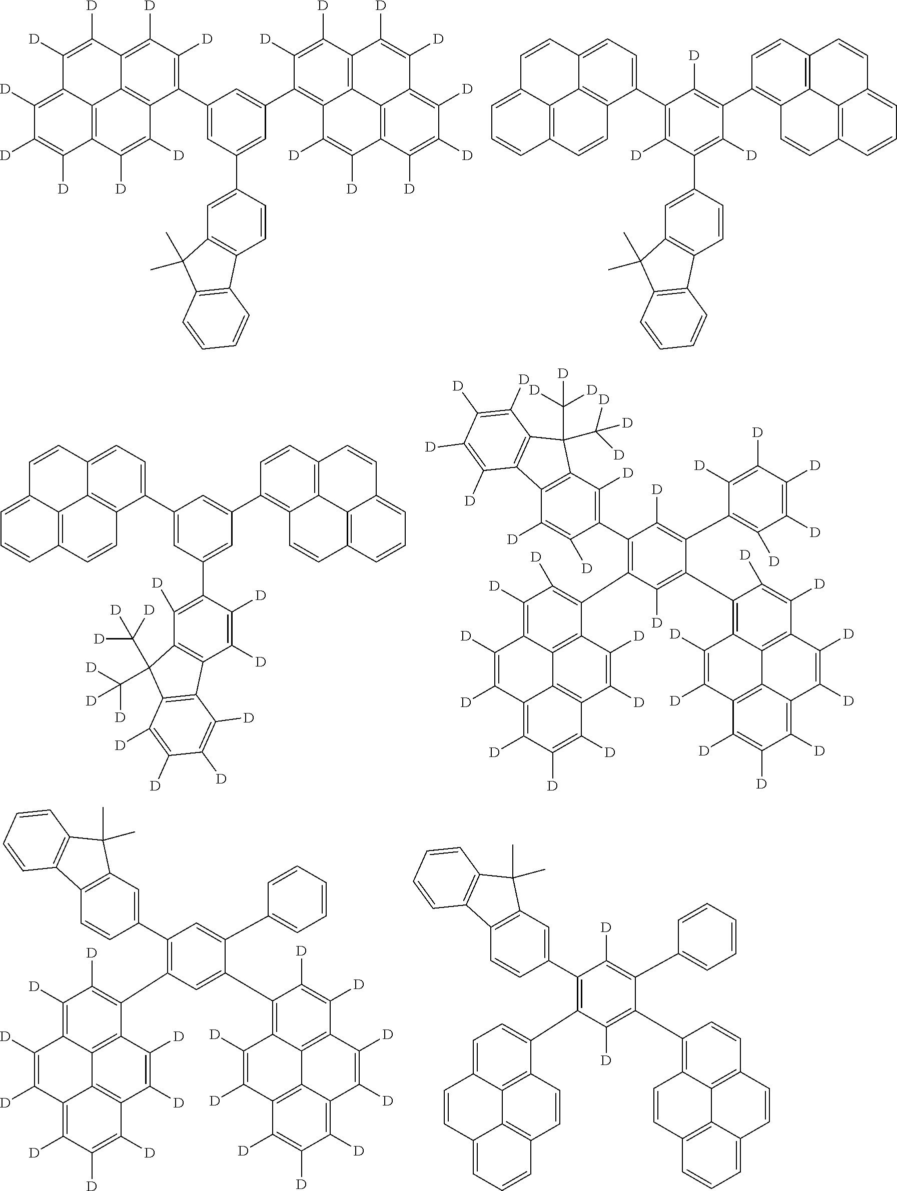

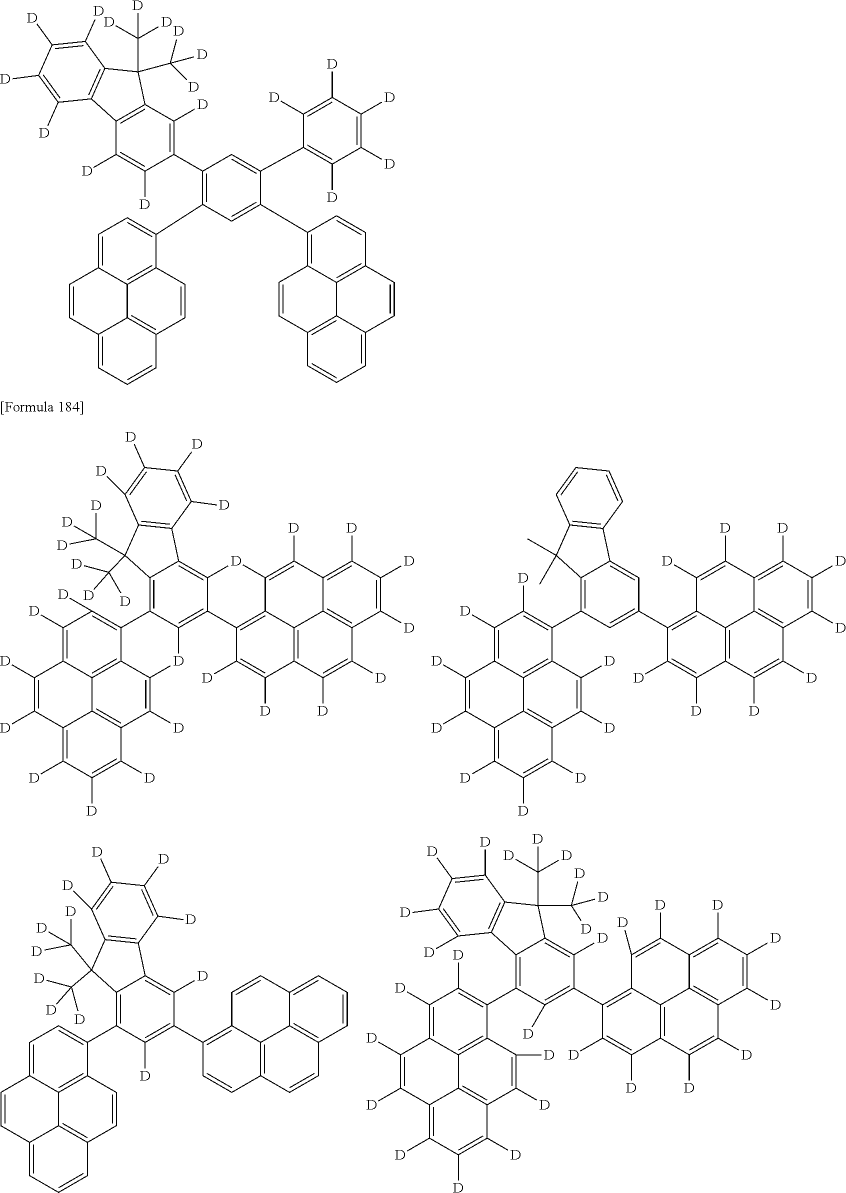

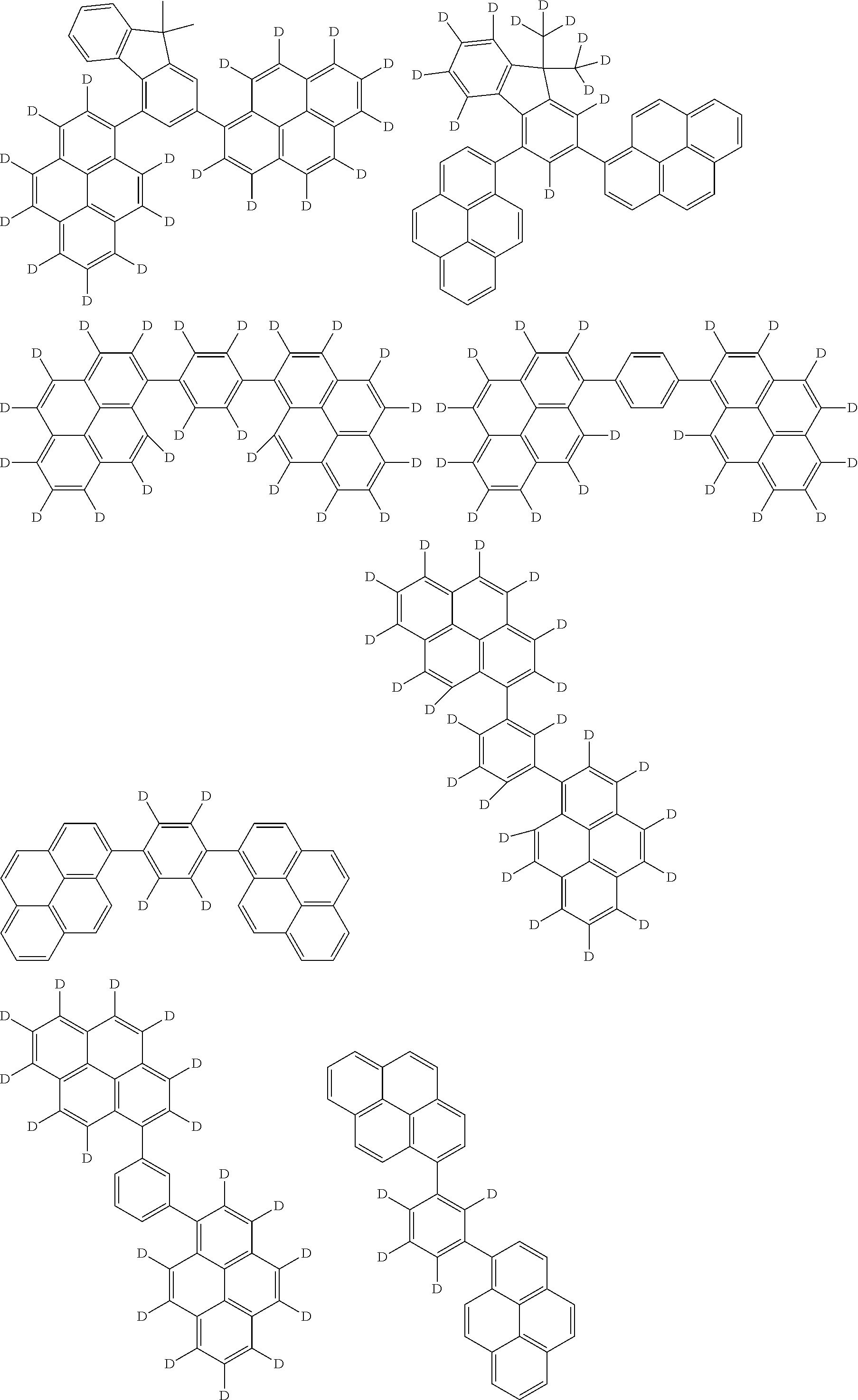

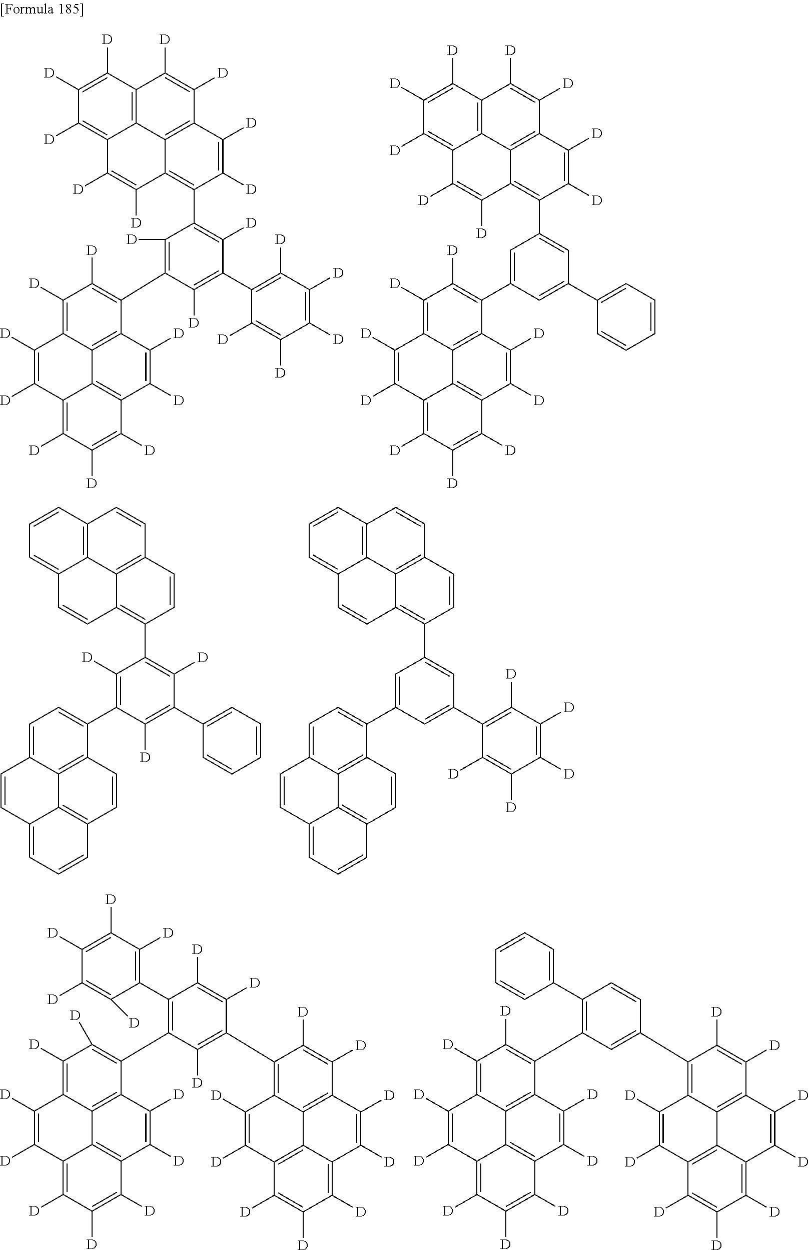

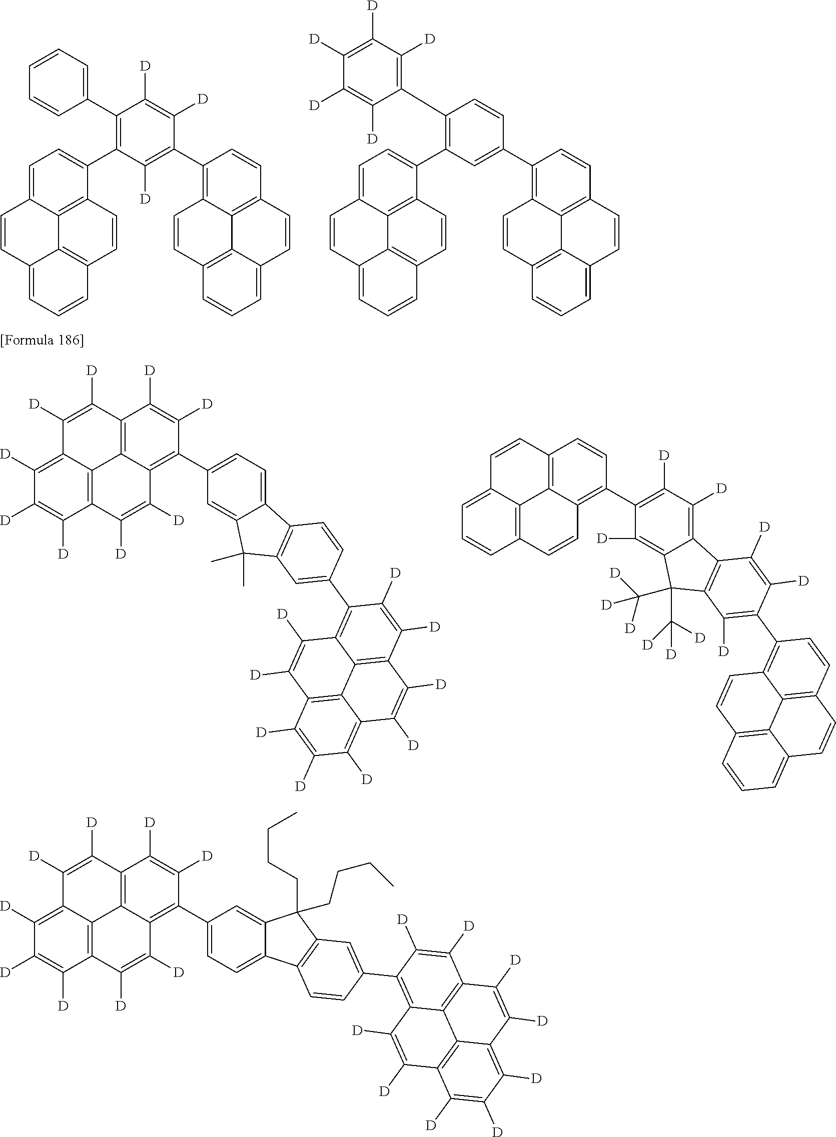

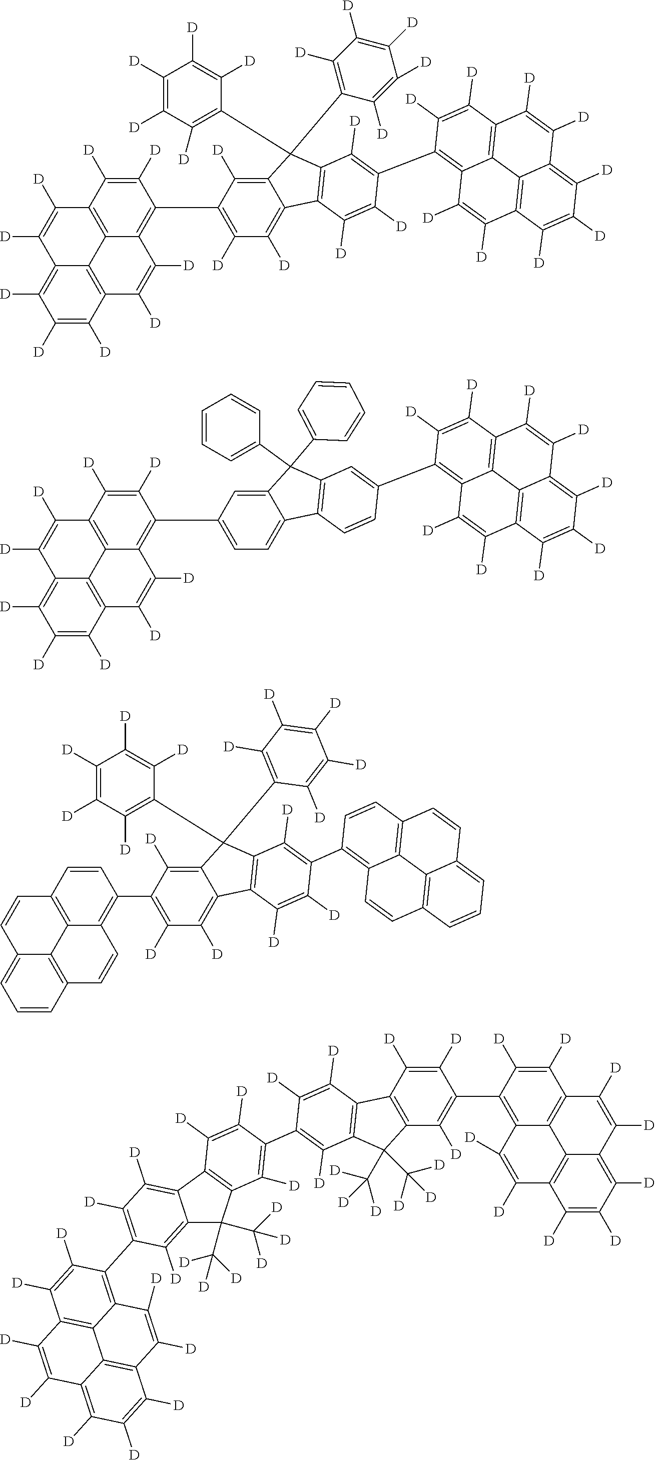

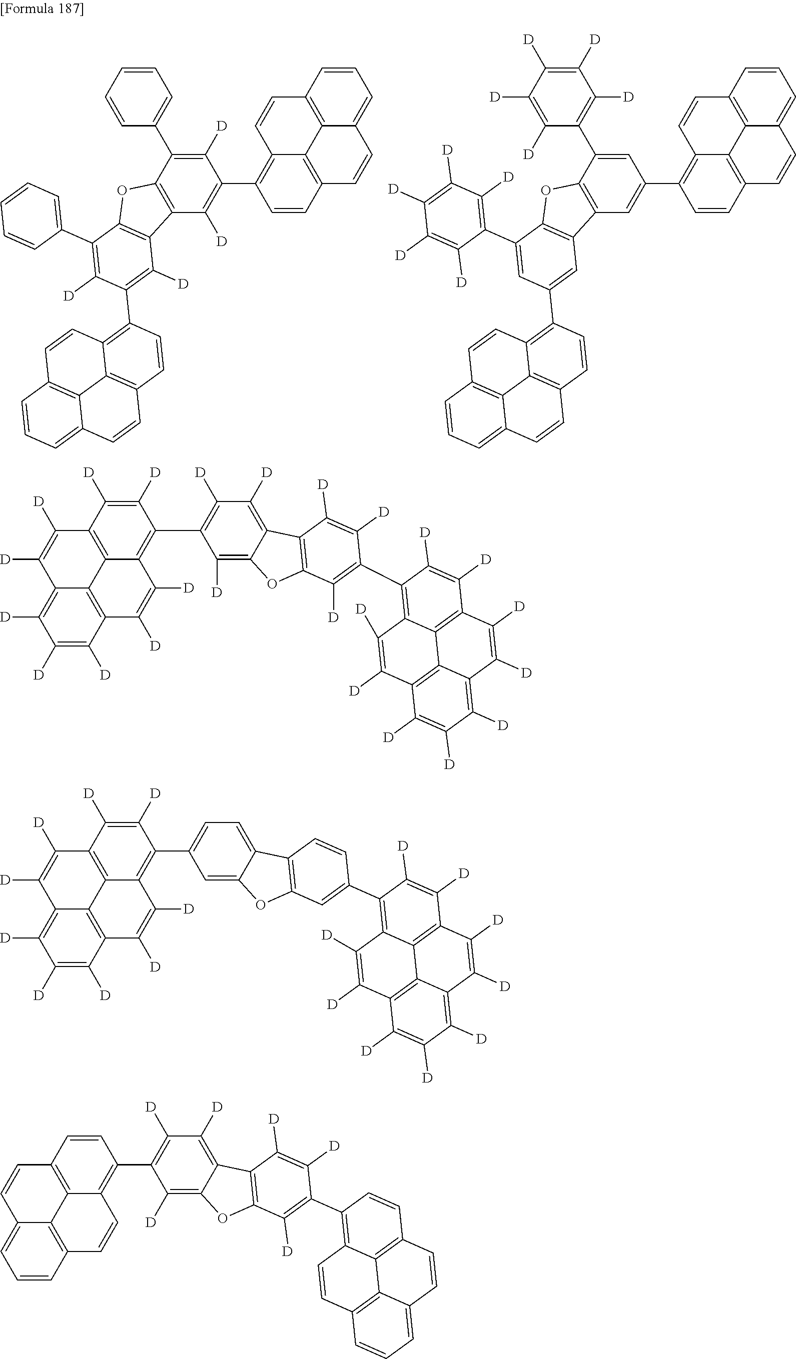

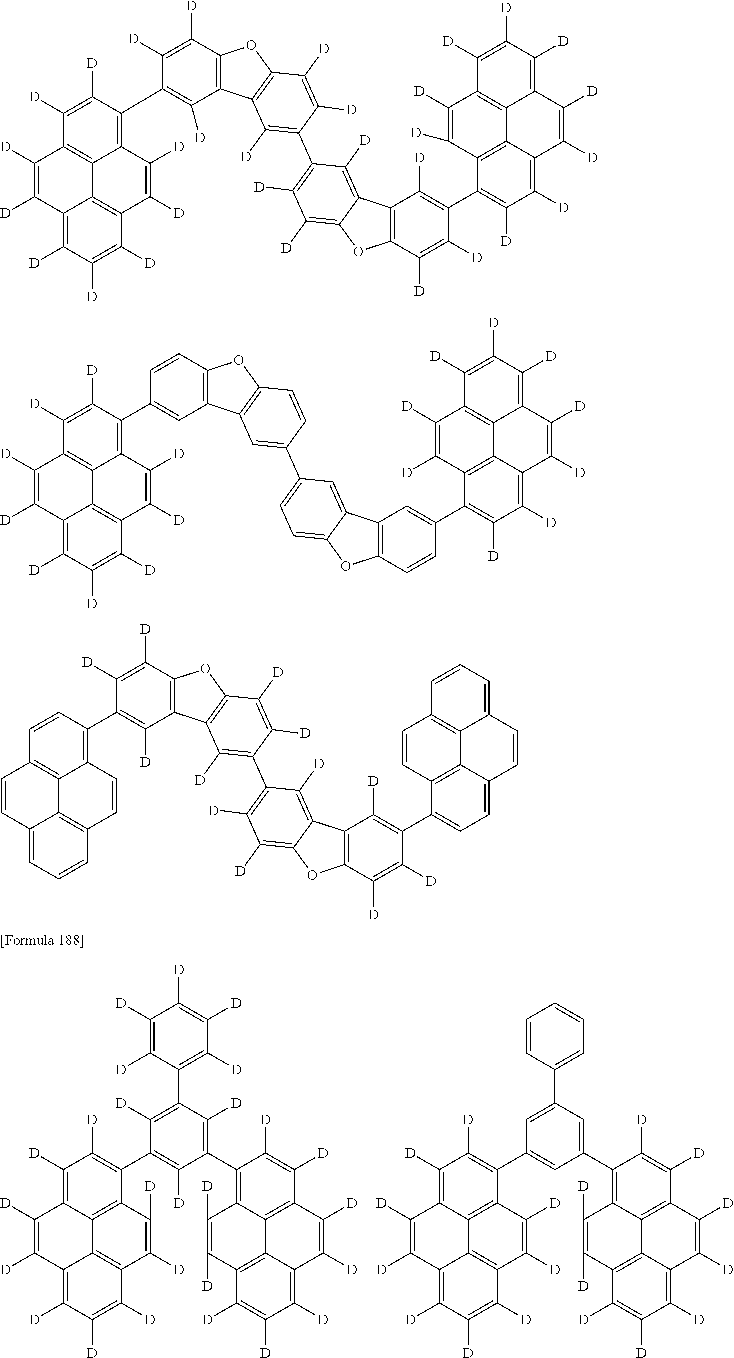

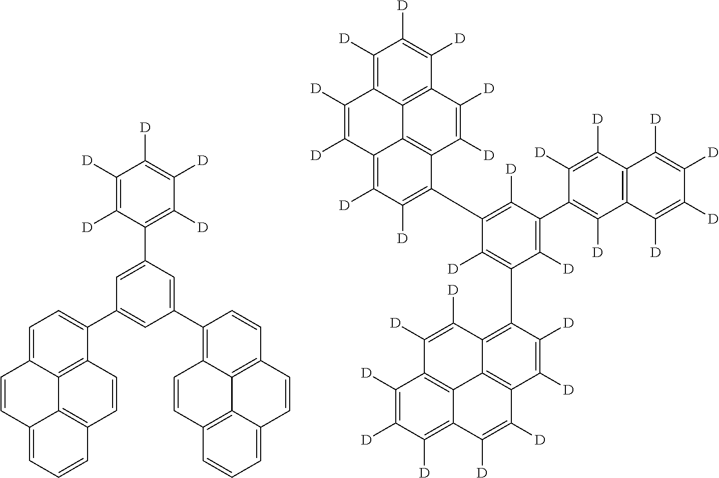 [SMILES: [2H]c1c([2H])c([2H])c(-c2cc(-c3ccc4ccc5cccc6ccc3c4c56)cc(-c3ccc4ccc5cccc6ccc3c4c56)c2)c([2H])c1[2H] ; [2H]c1c(-c2c([2H])c([2H])c3c([2H])c([2H])c([2H])c([2H])c3c2[2H])c([2H])c(-c2c([2H])c([2H])c3c([2H])c([2H])c4c([2H])c([2H])c([2H])c5c([2H])c([2H])c2c3c45)c([2H])c1-c1c([2H])c([2H])c2c([2H])c([2H])c3c([2H])c([2H])c([2H])c4c([2H])c([2H])c1c2c34]

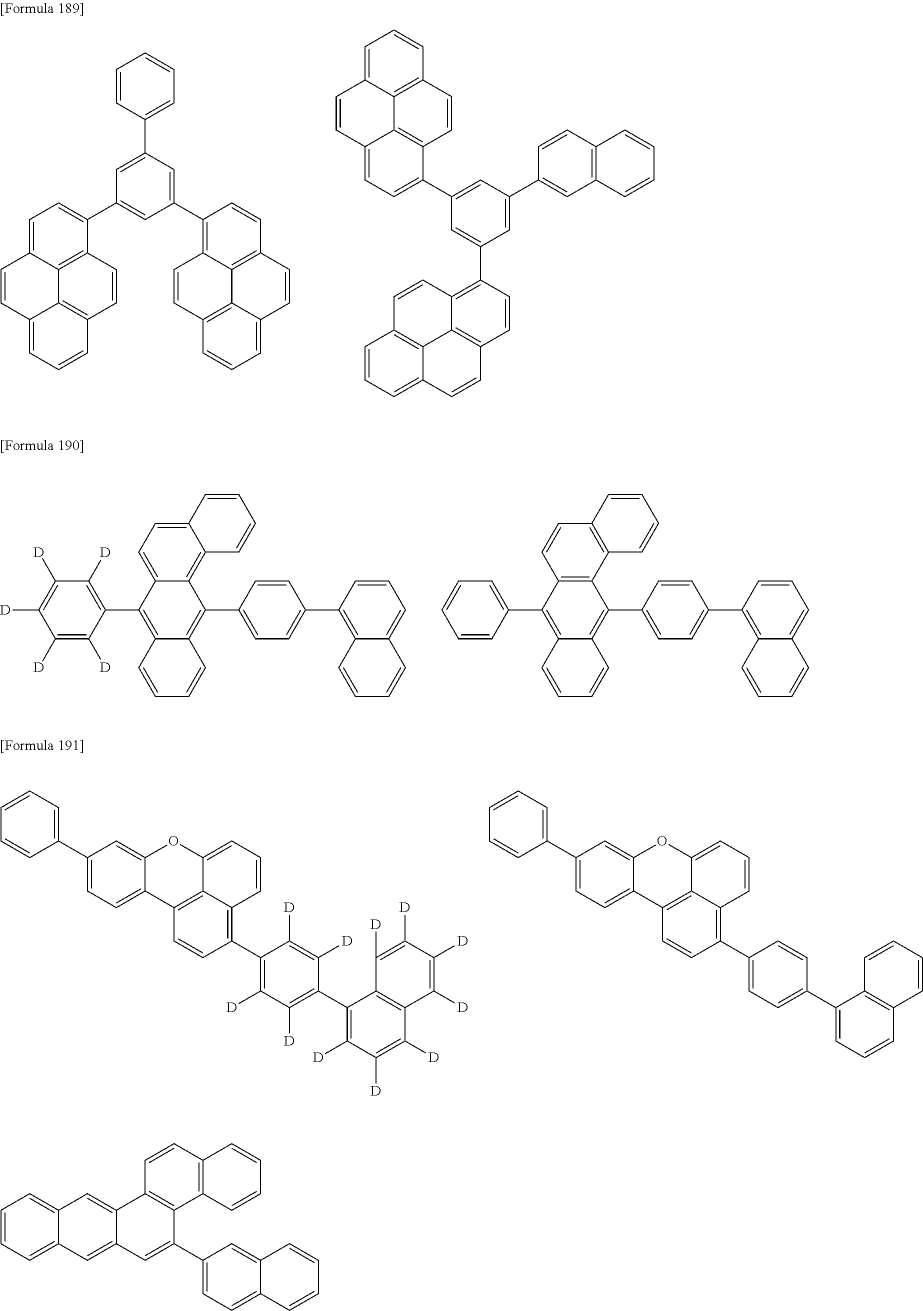

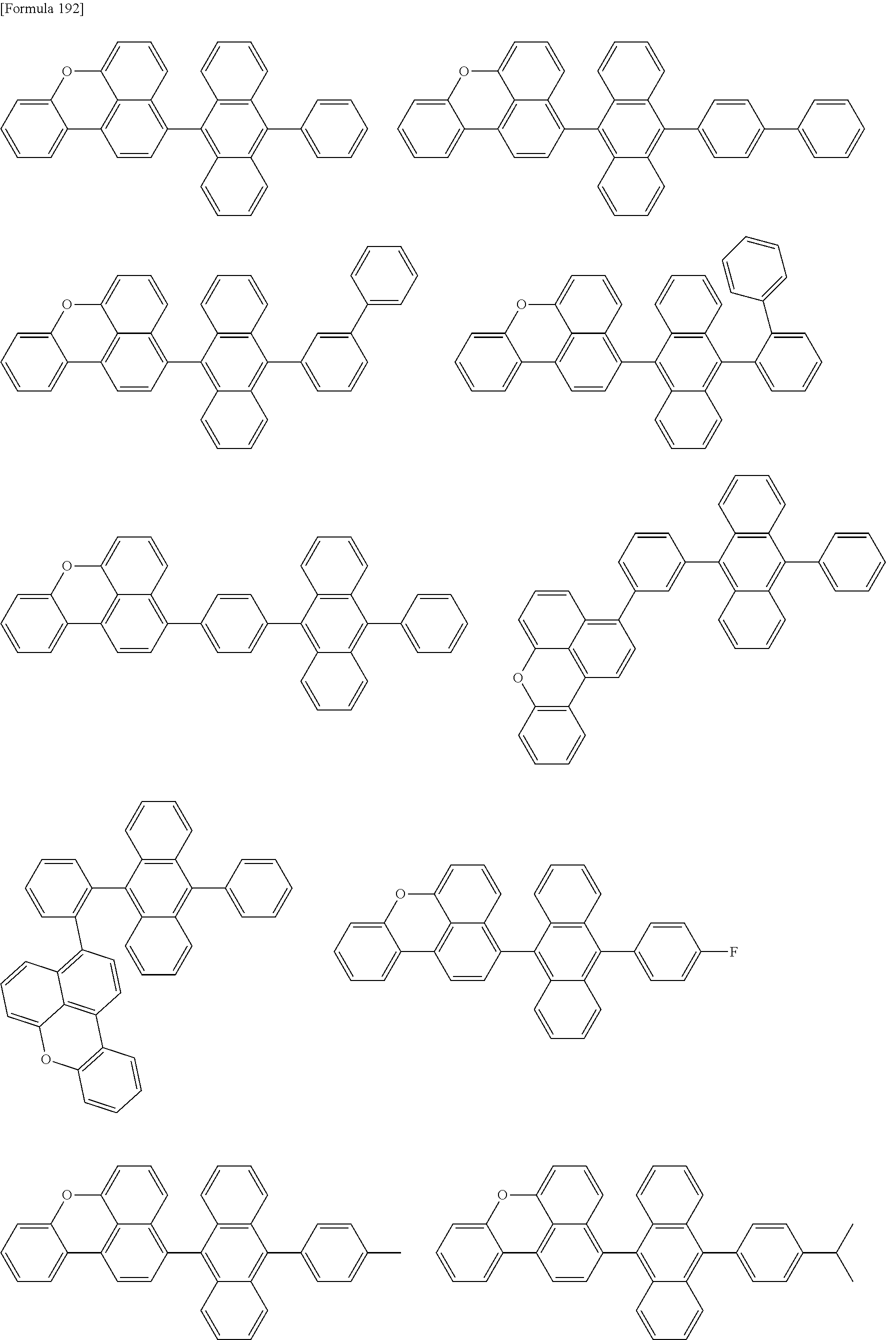

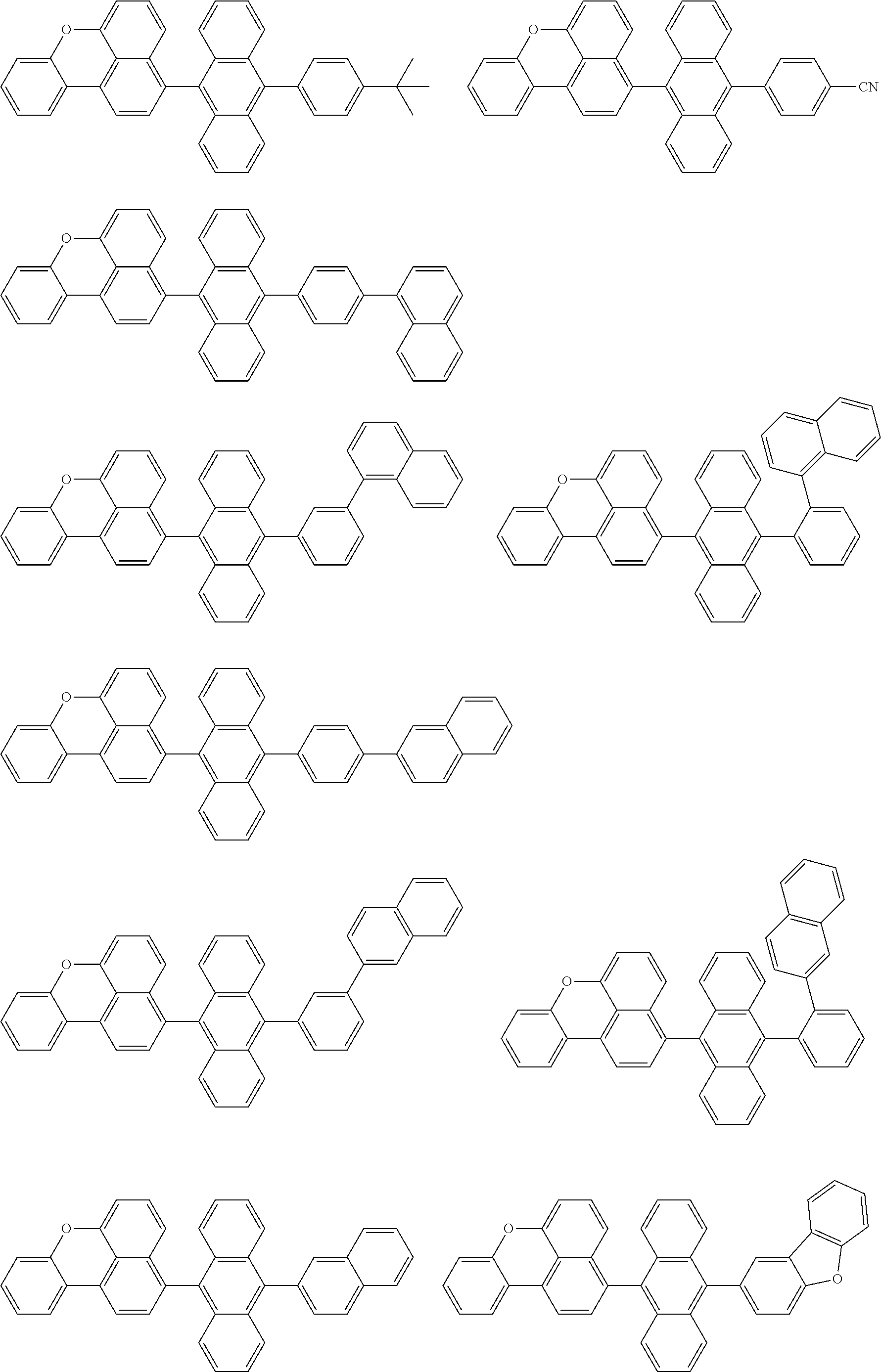

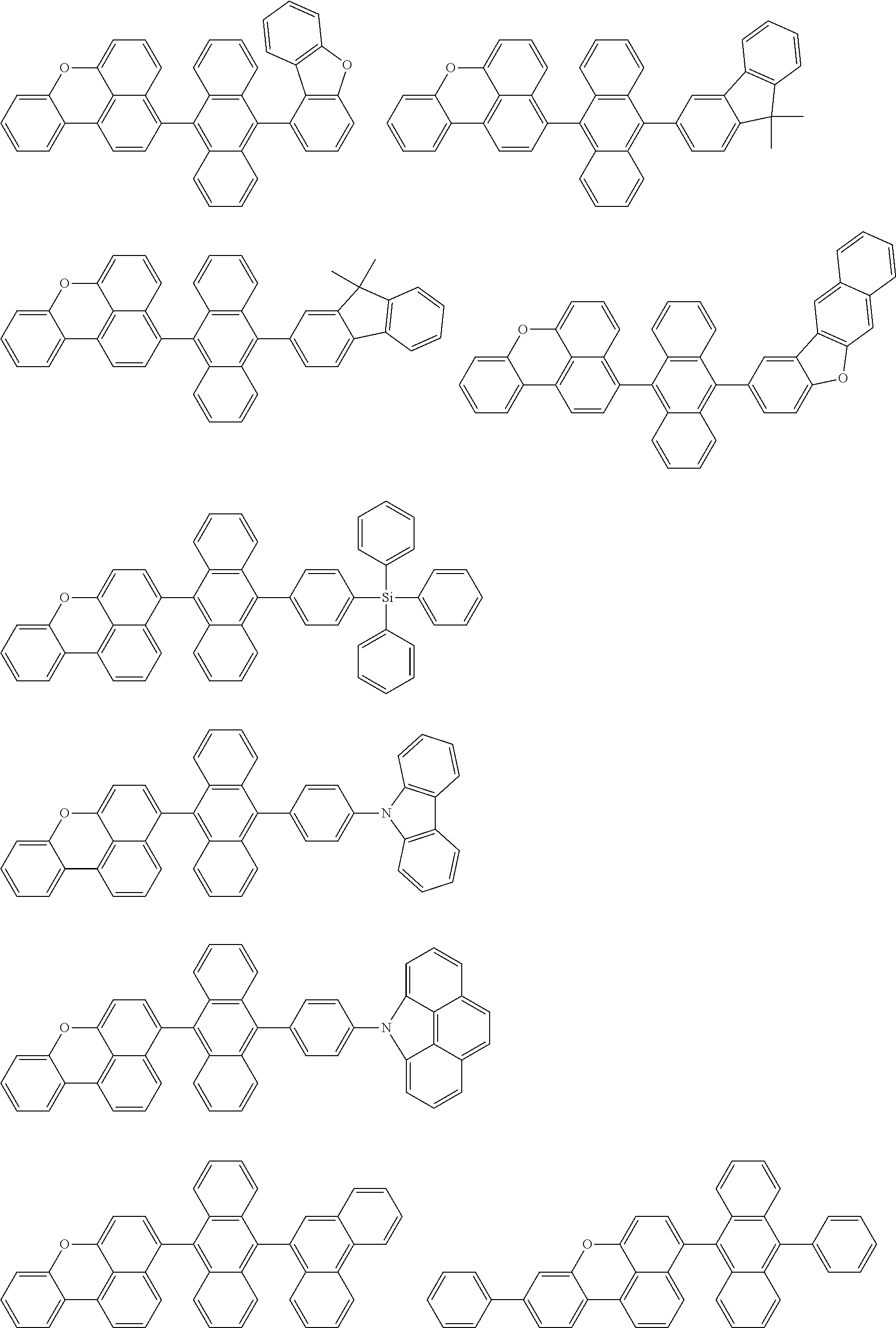

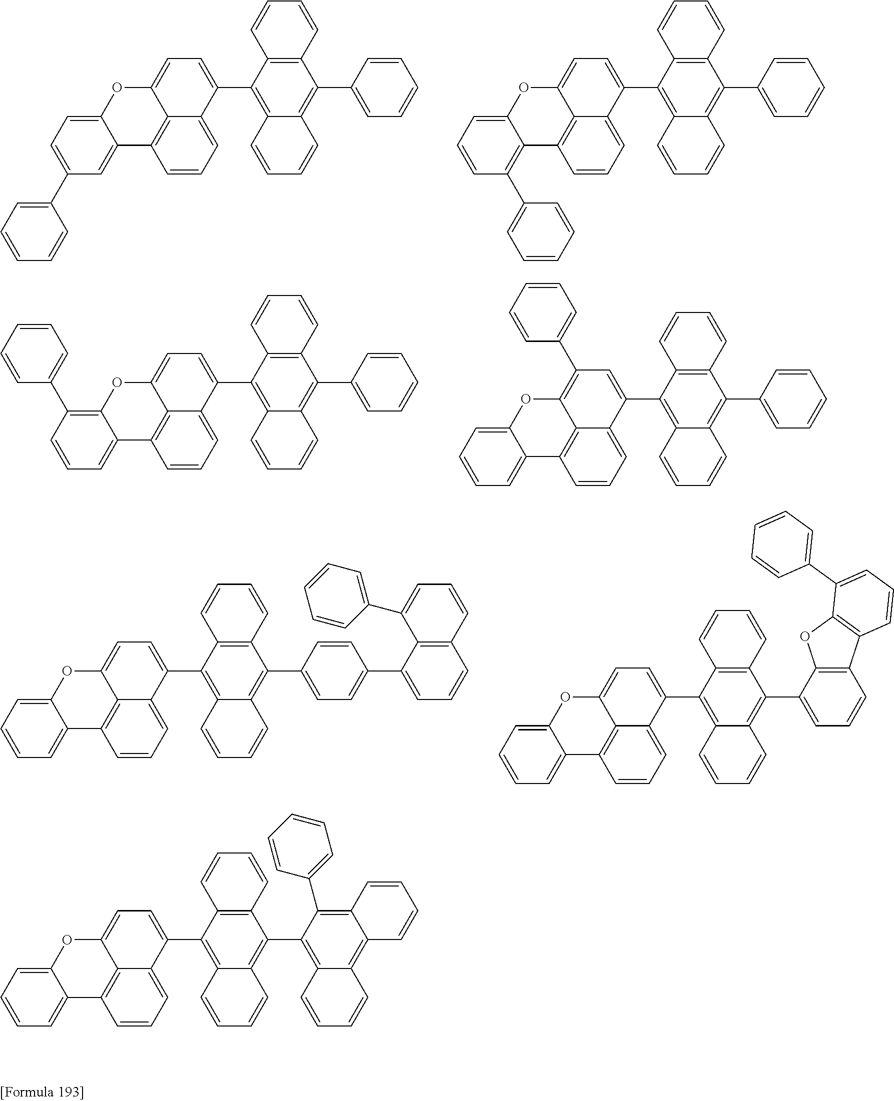

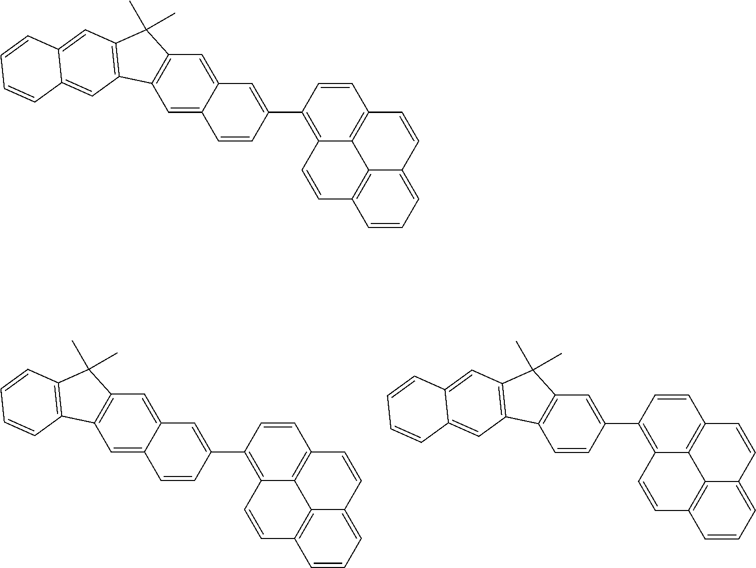

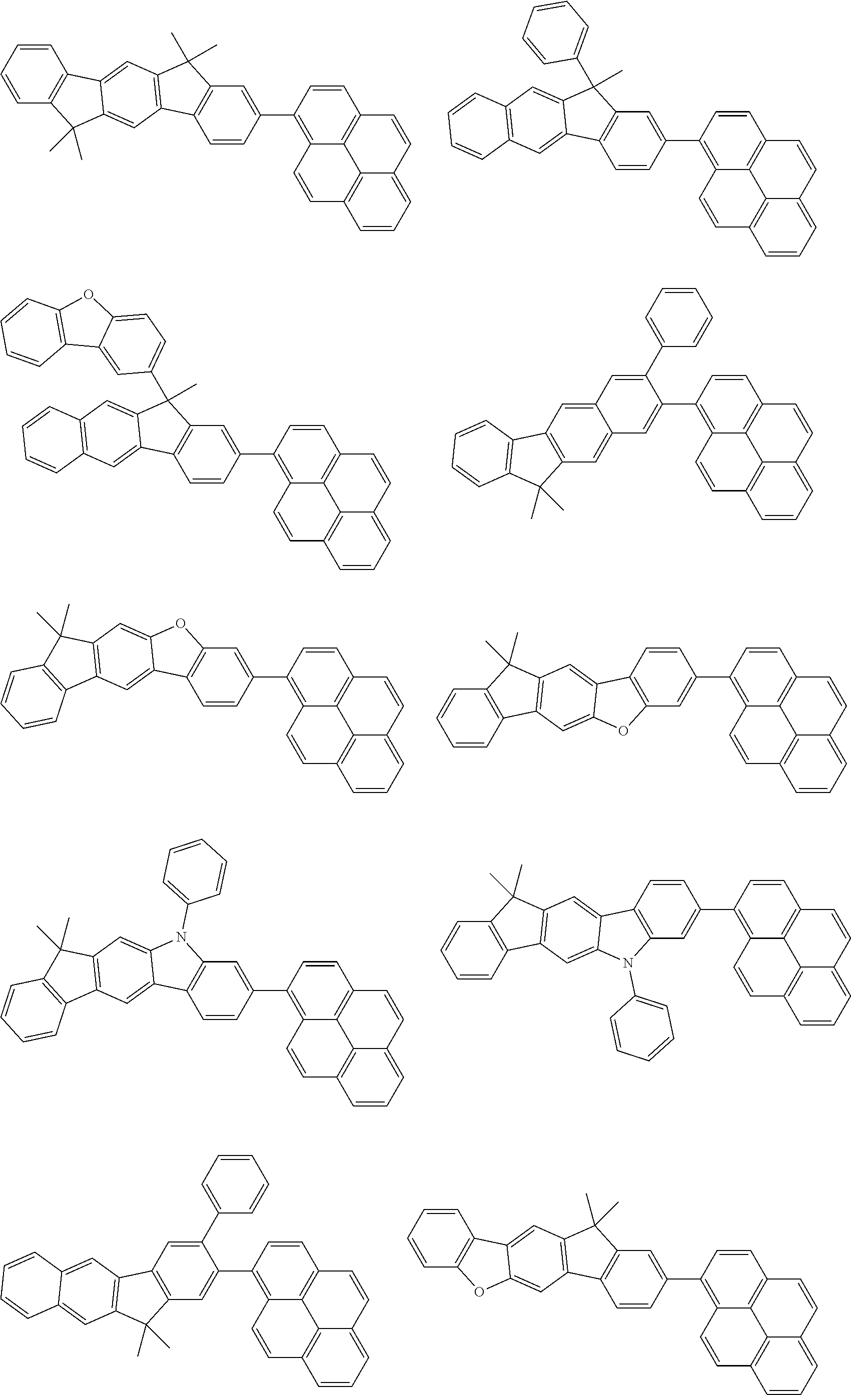

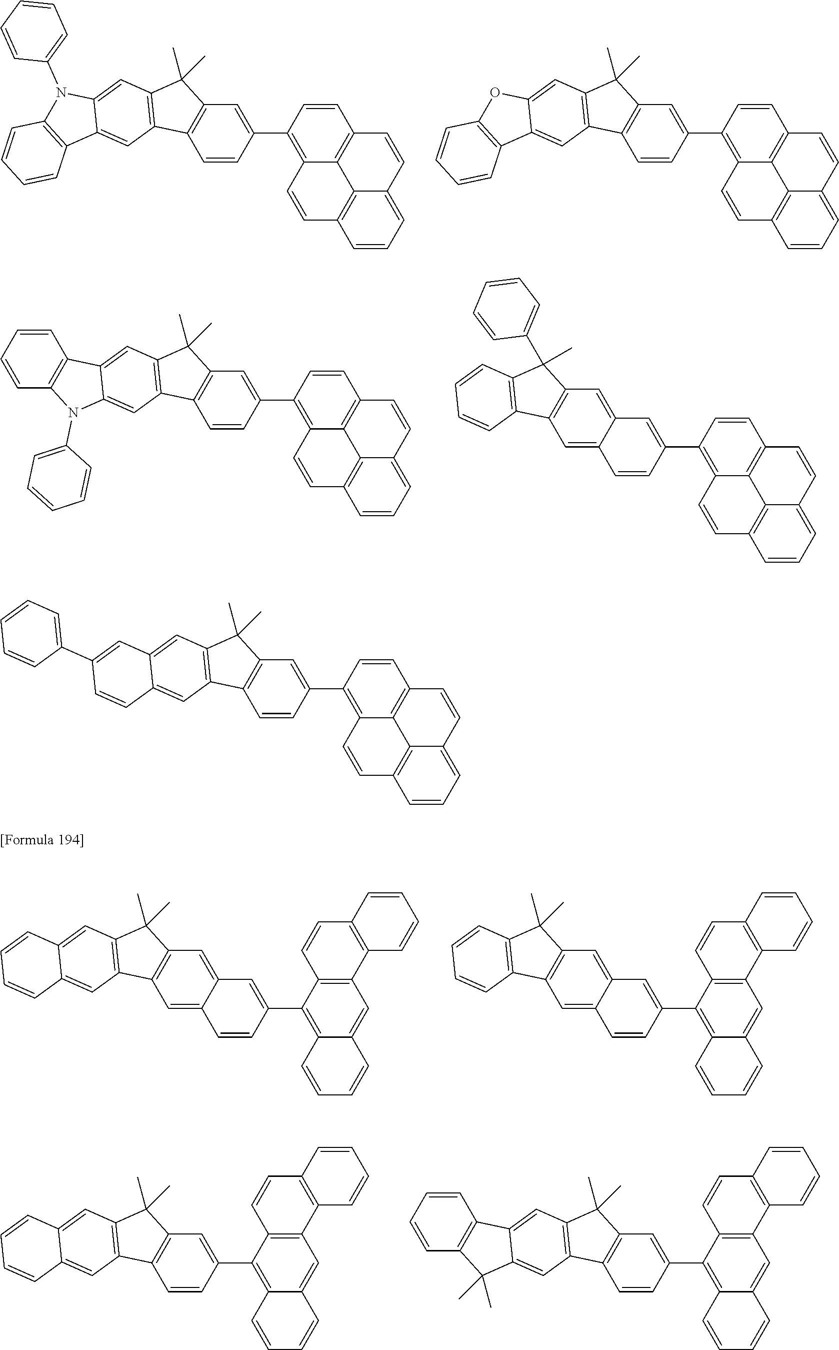

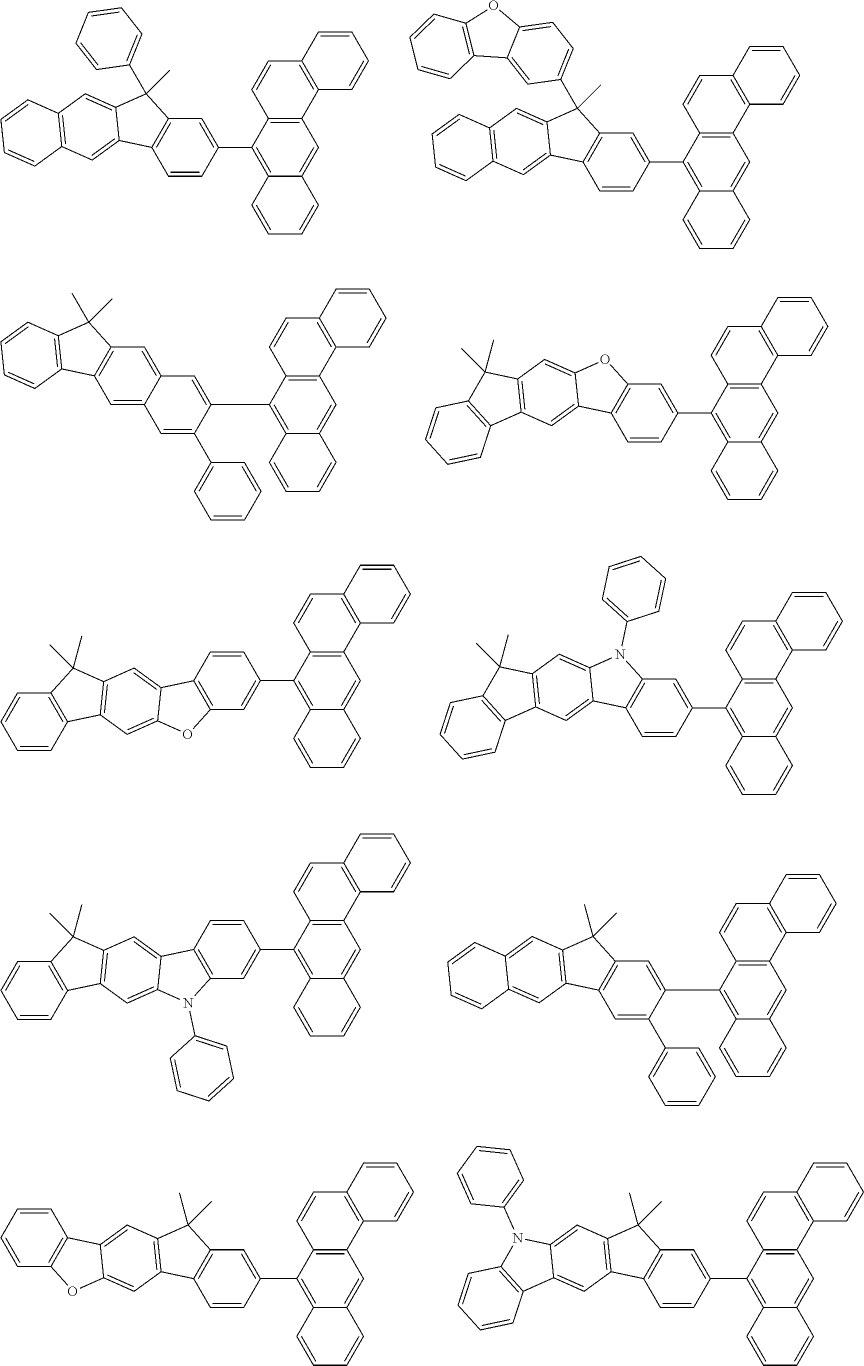

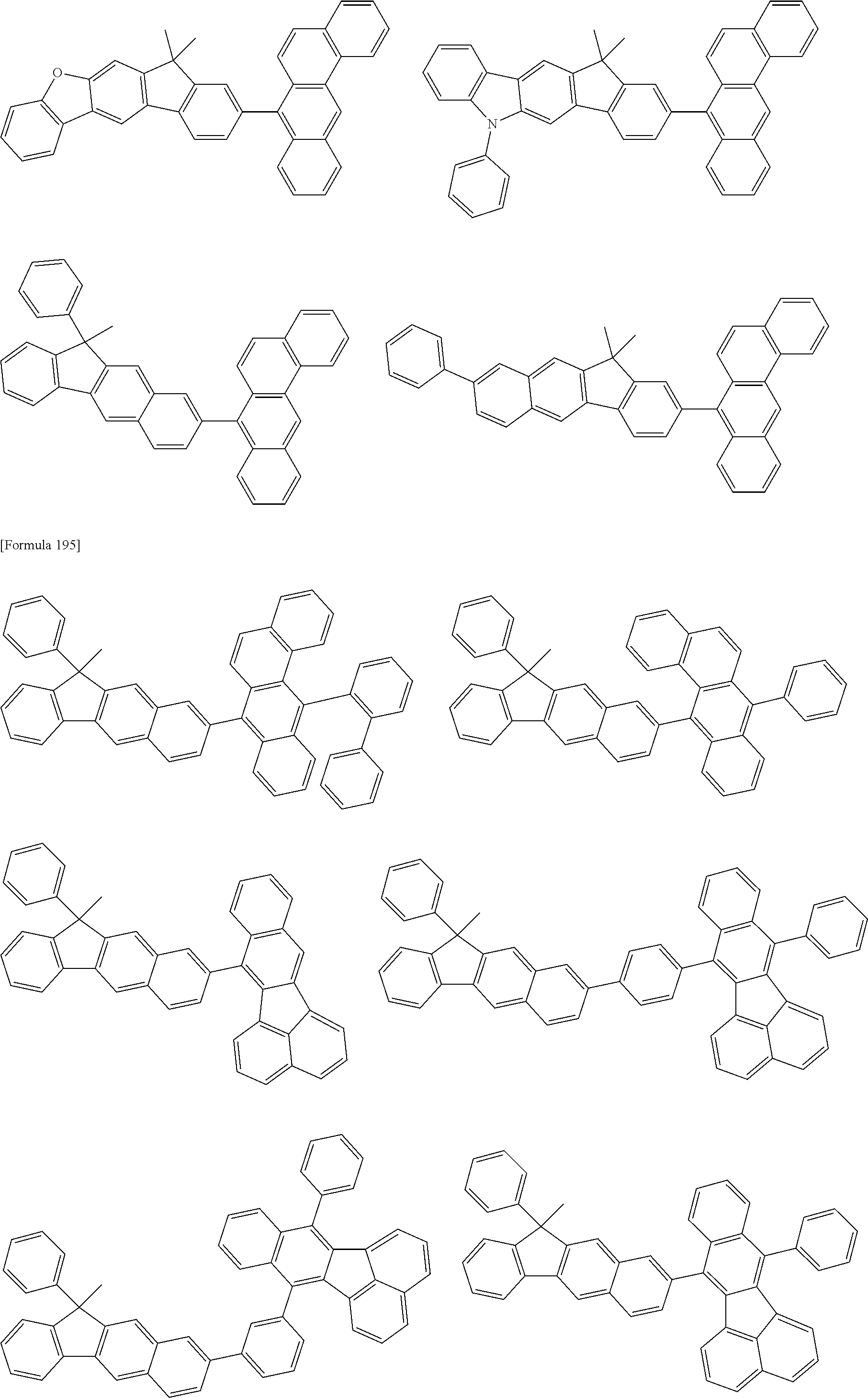

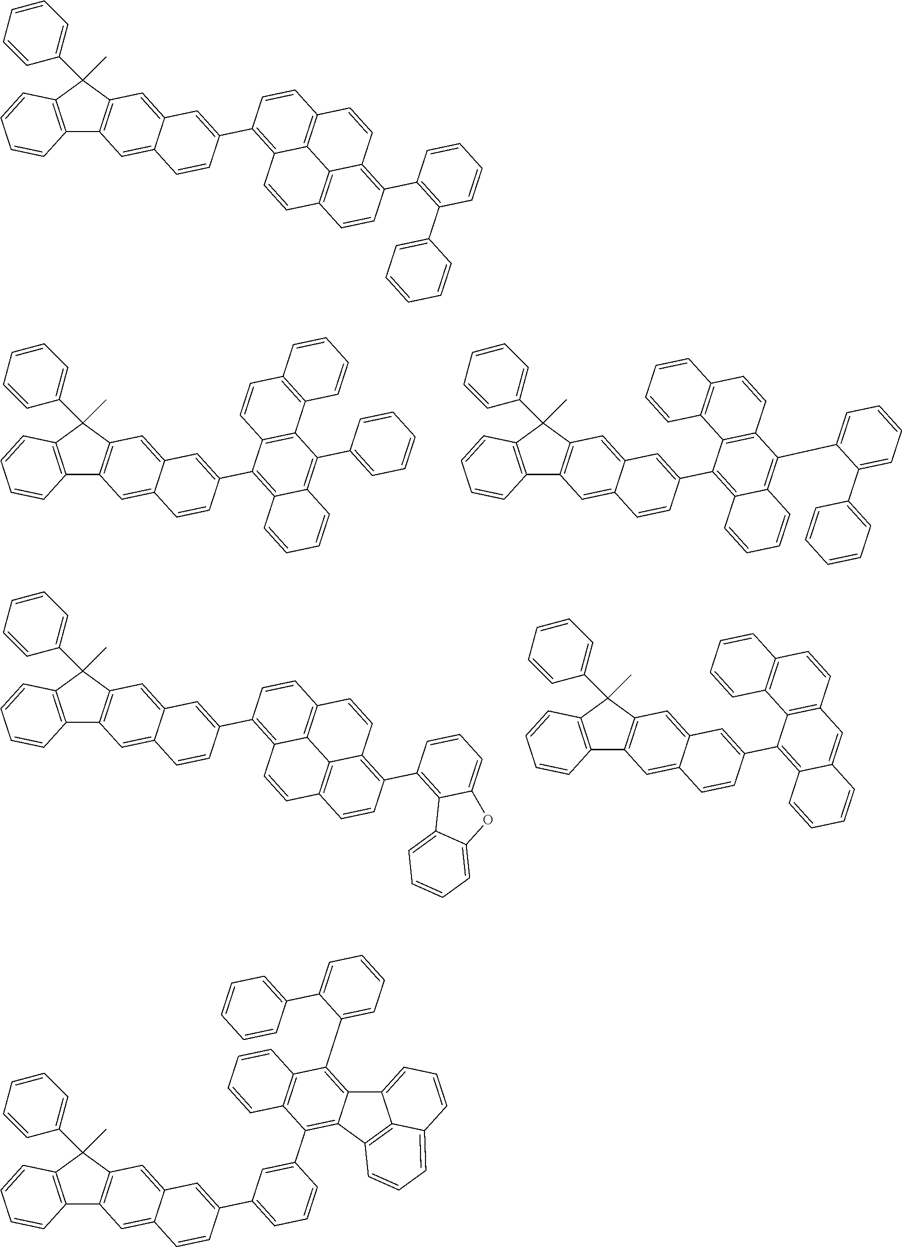

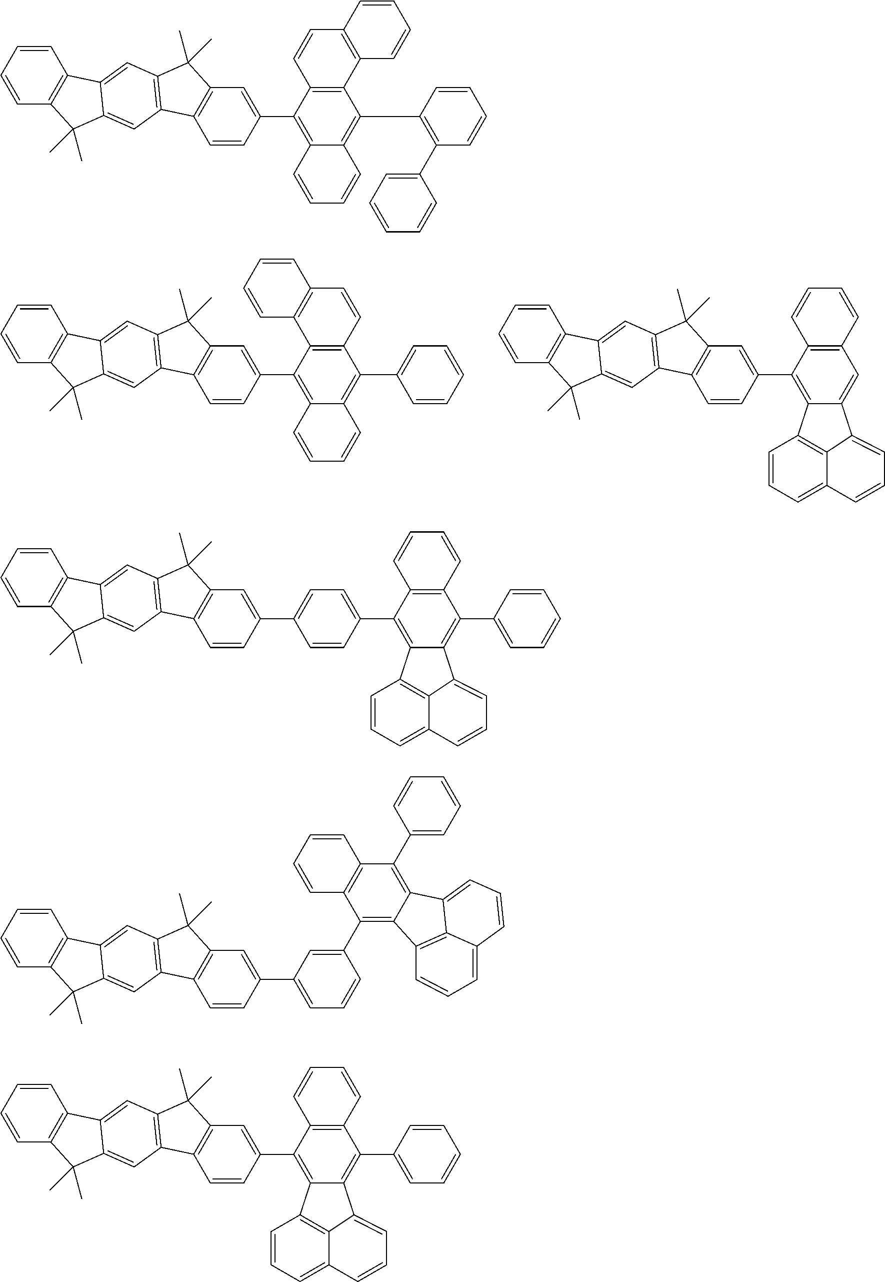

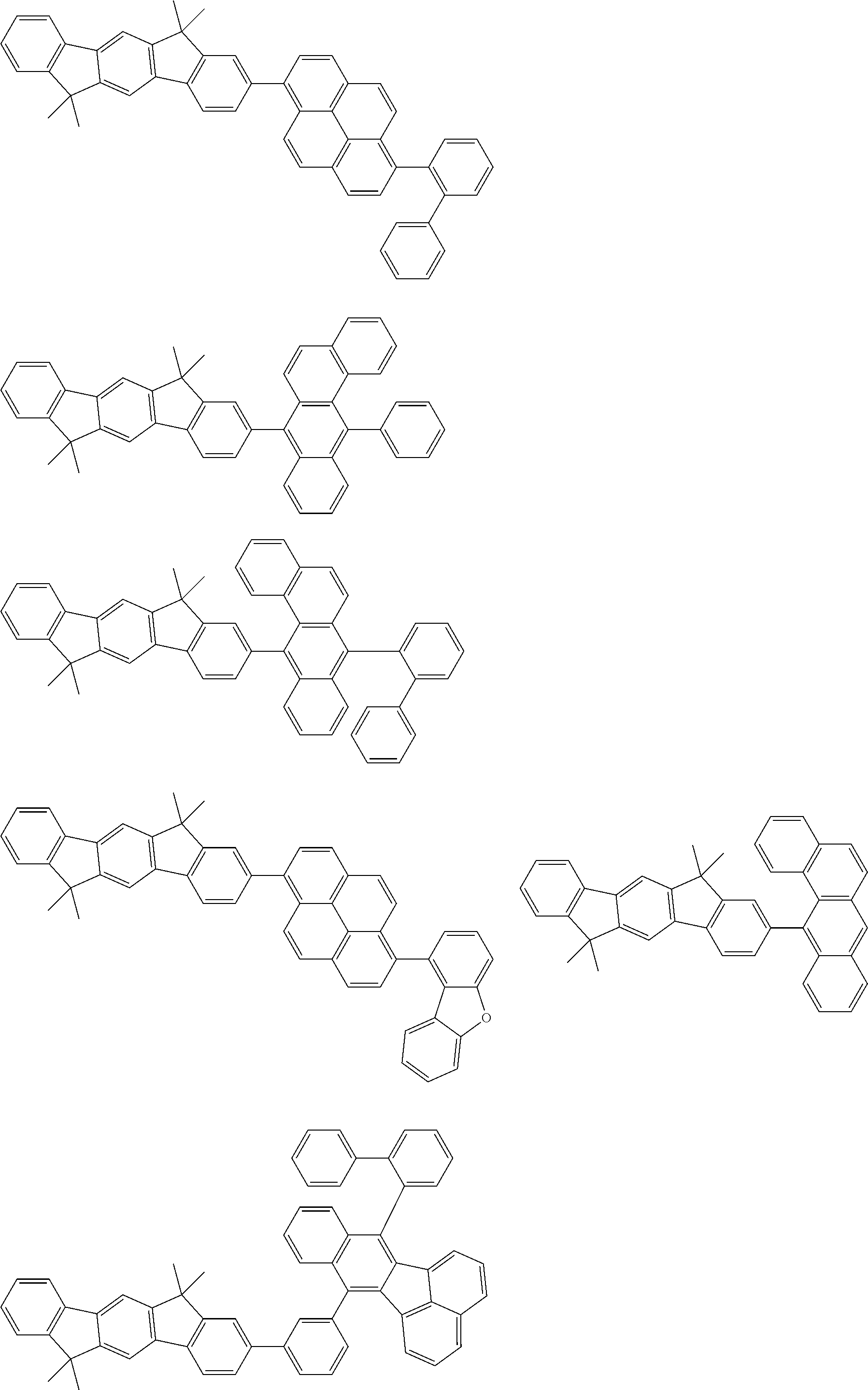

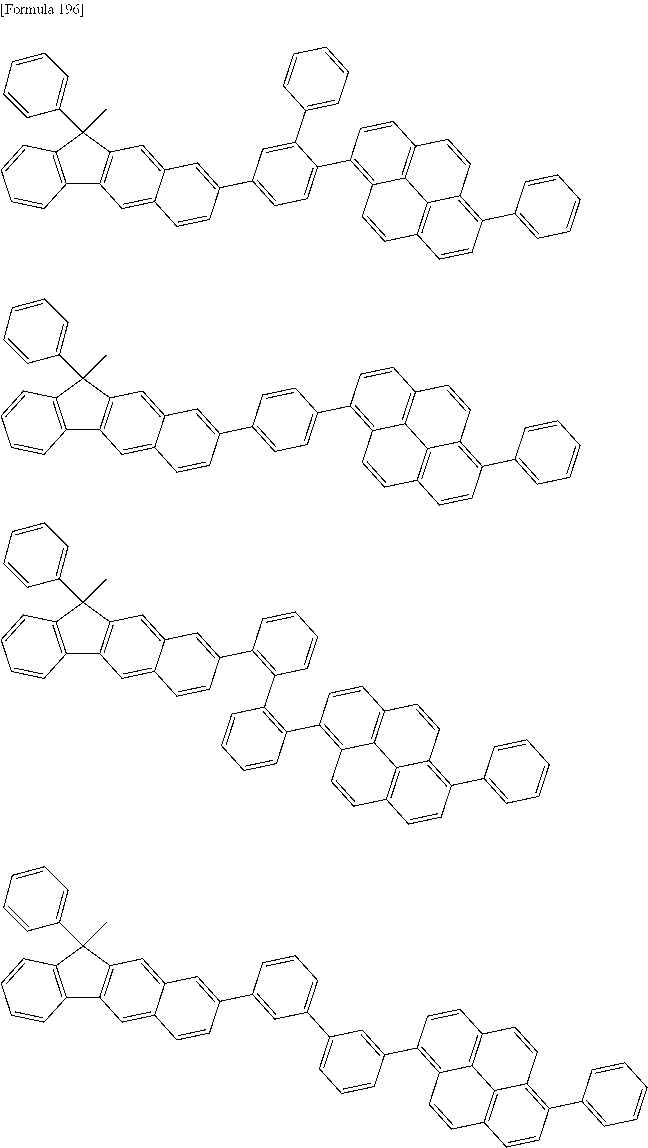

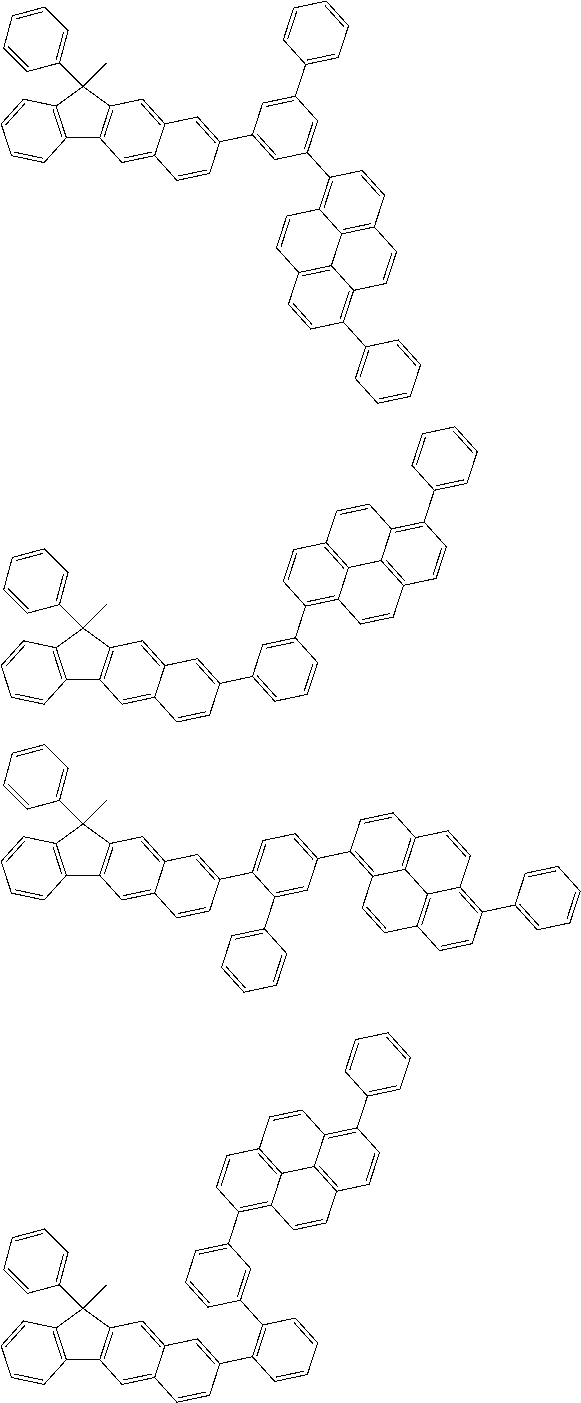

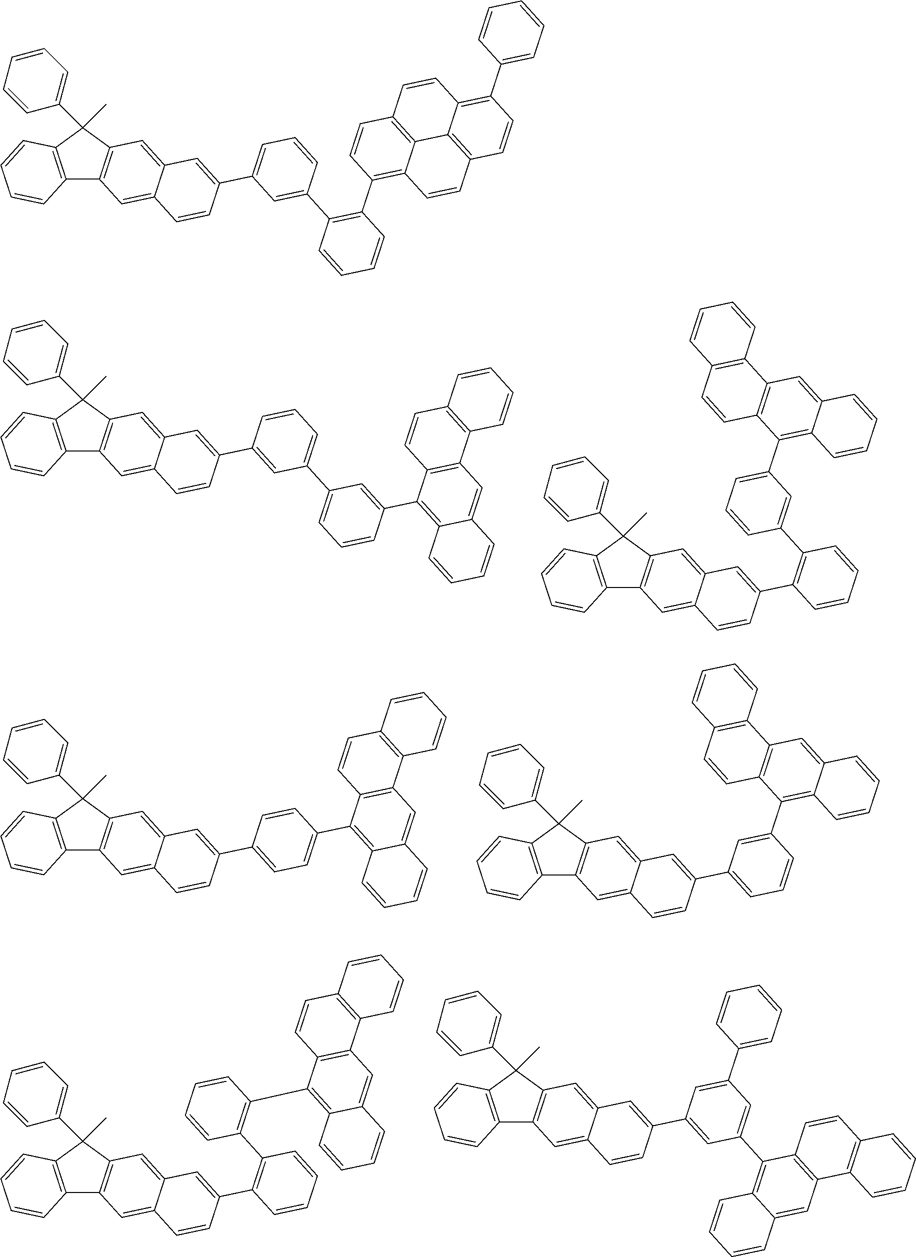

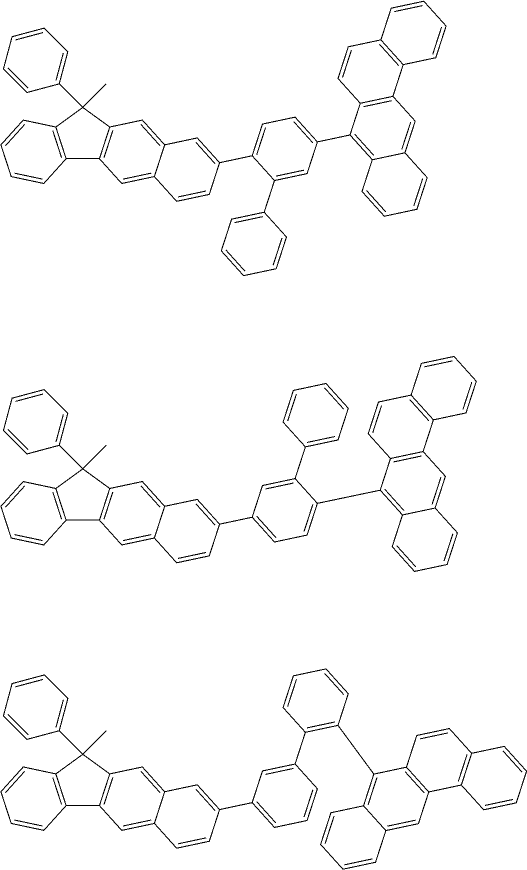

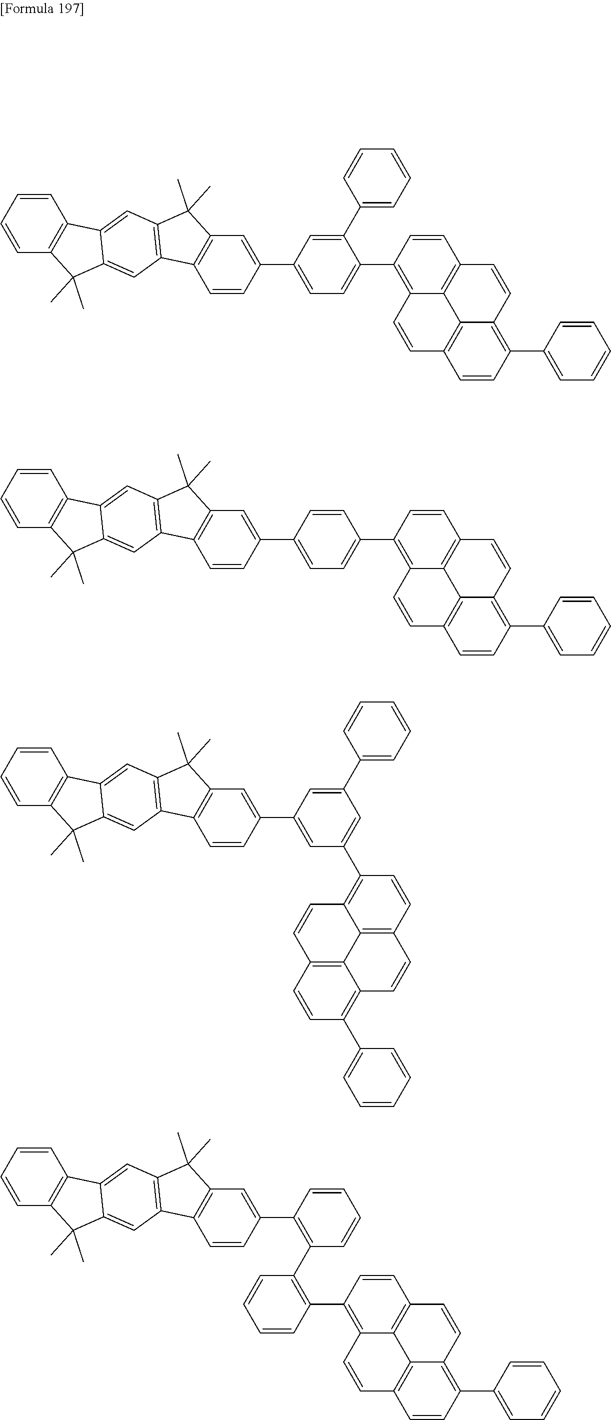

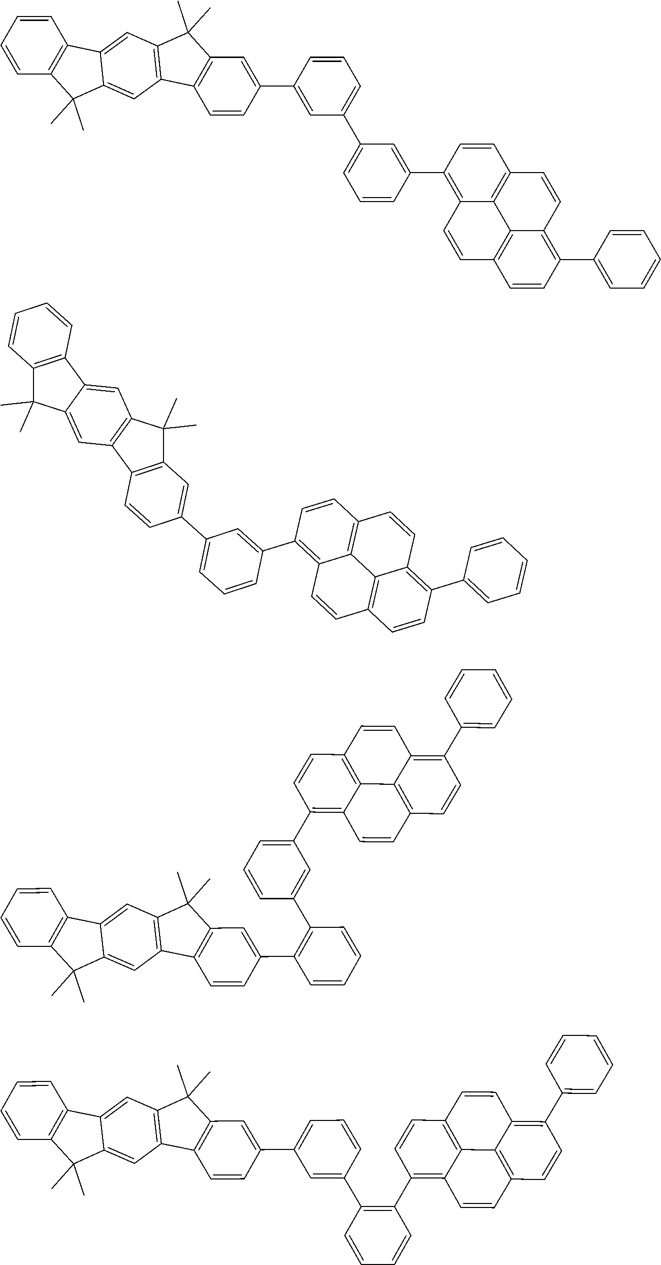

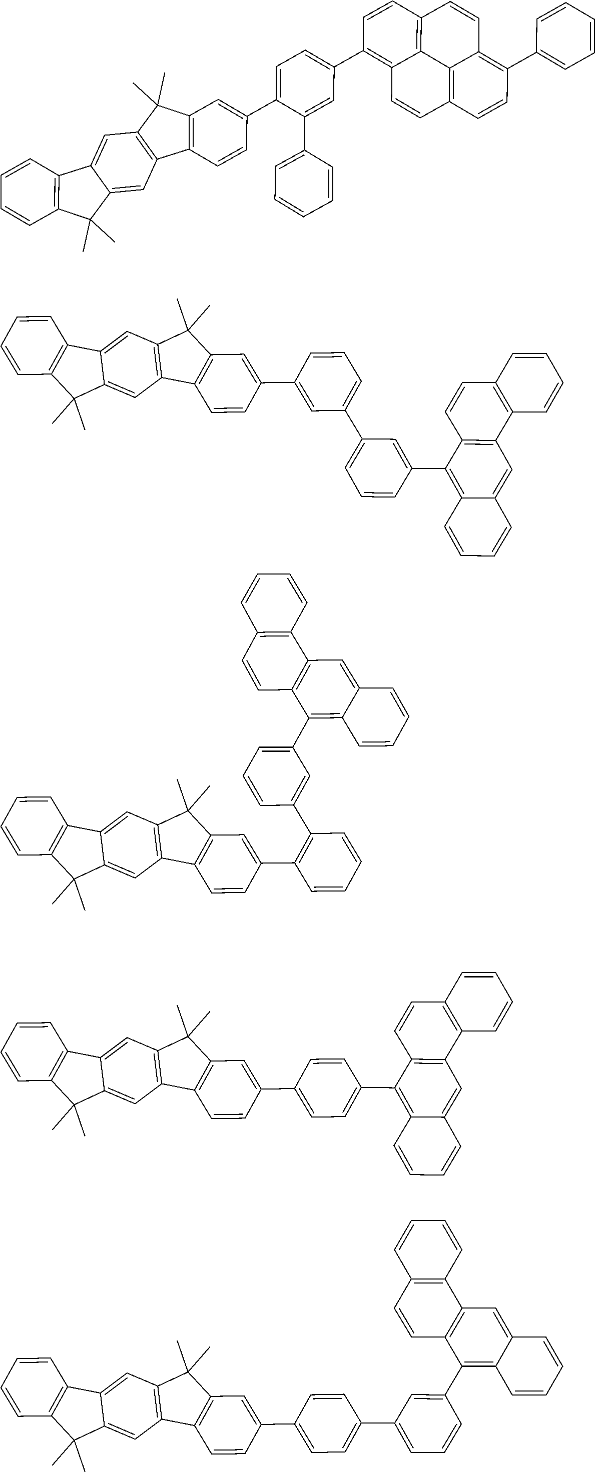

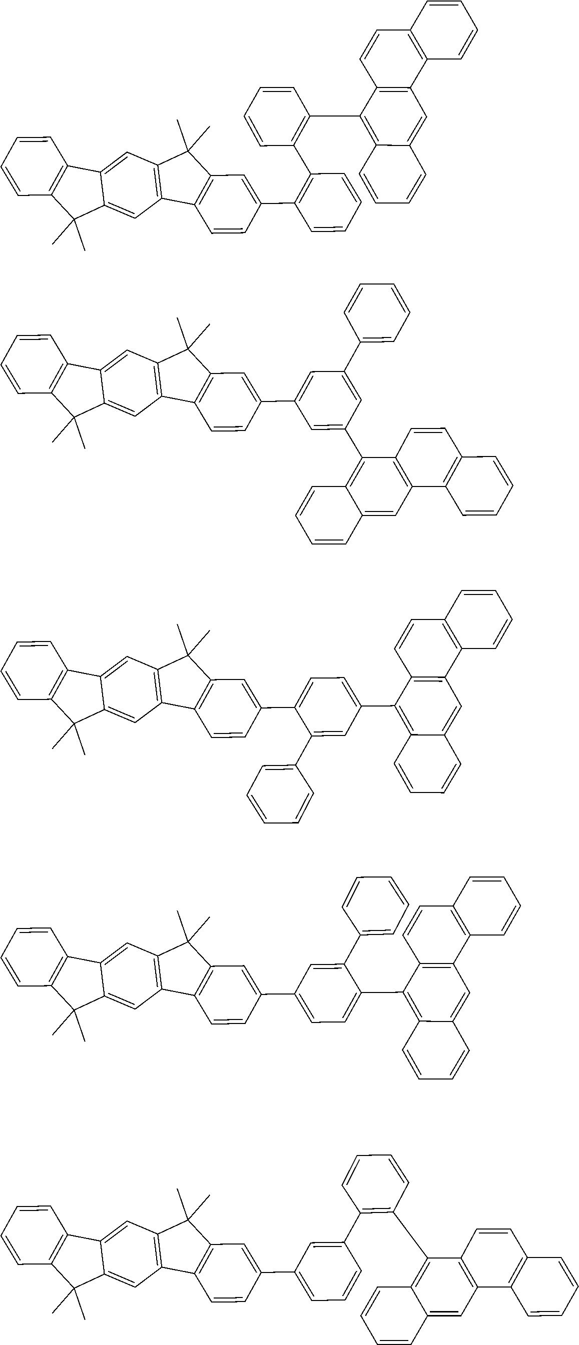

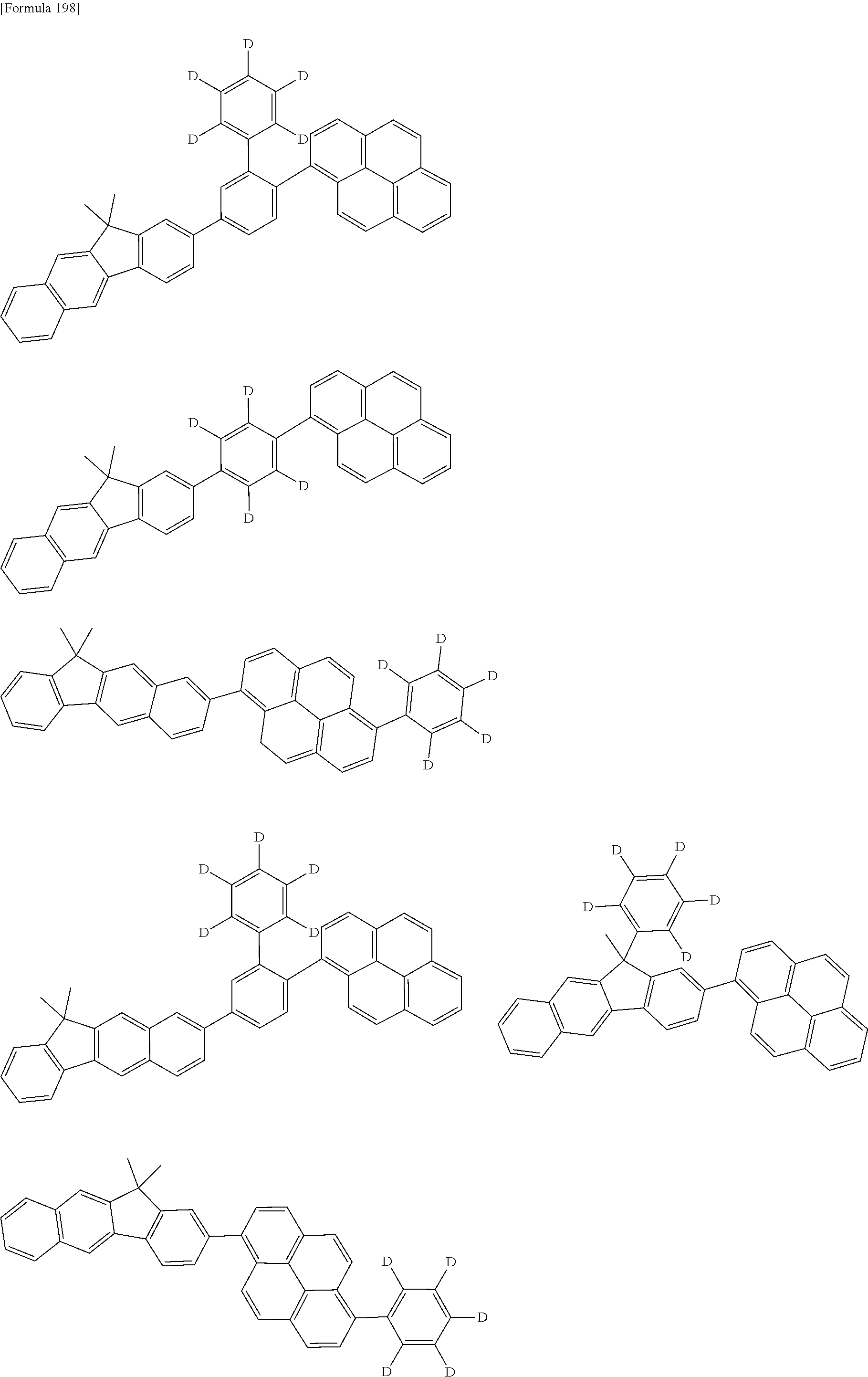

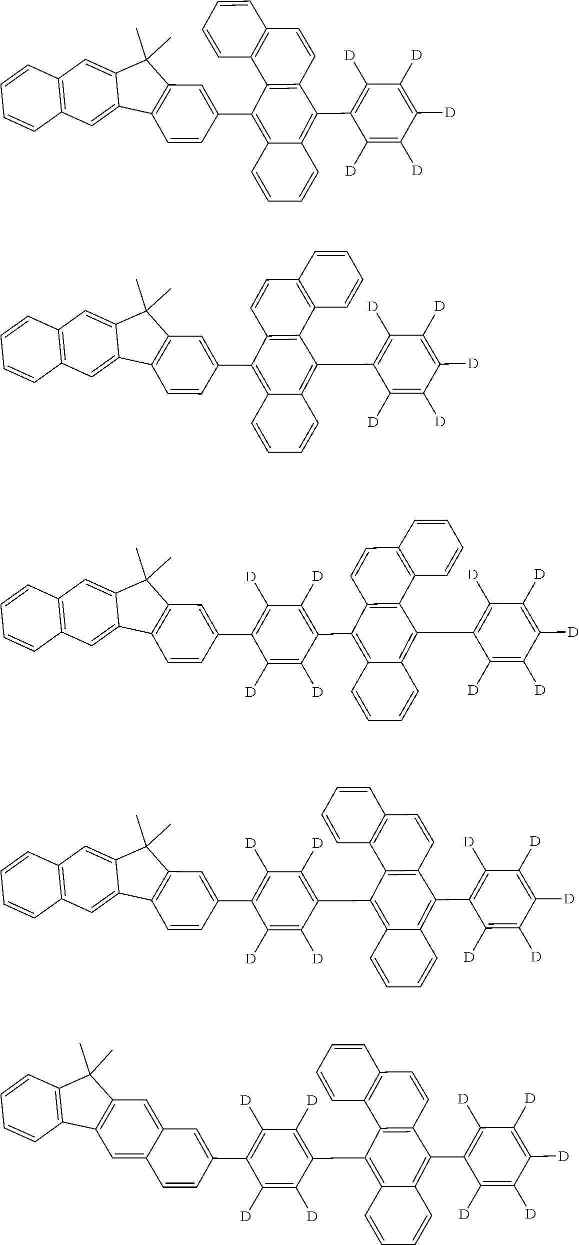

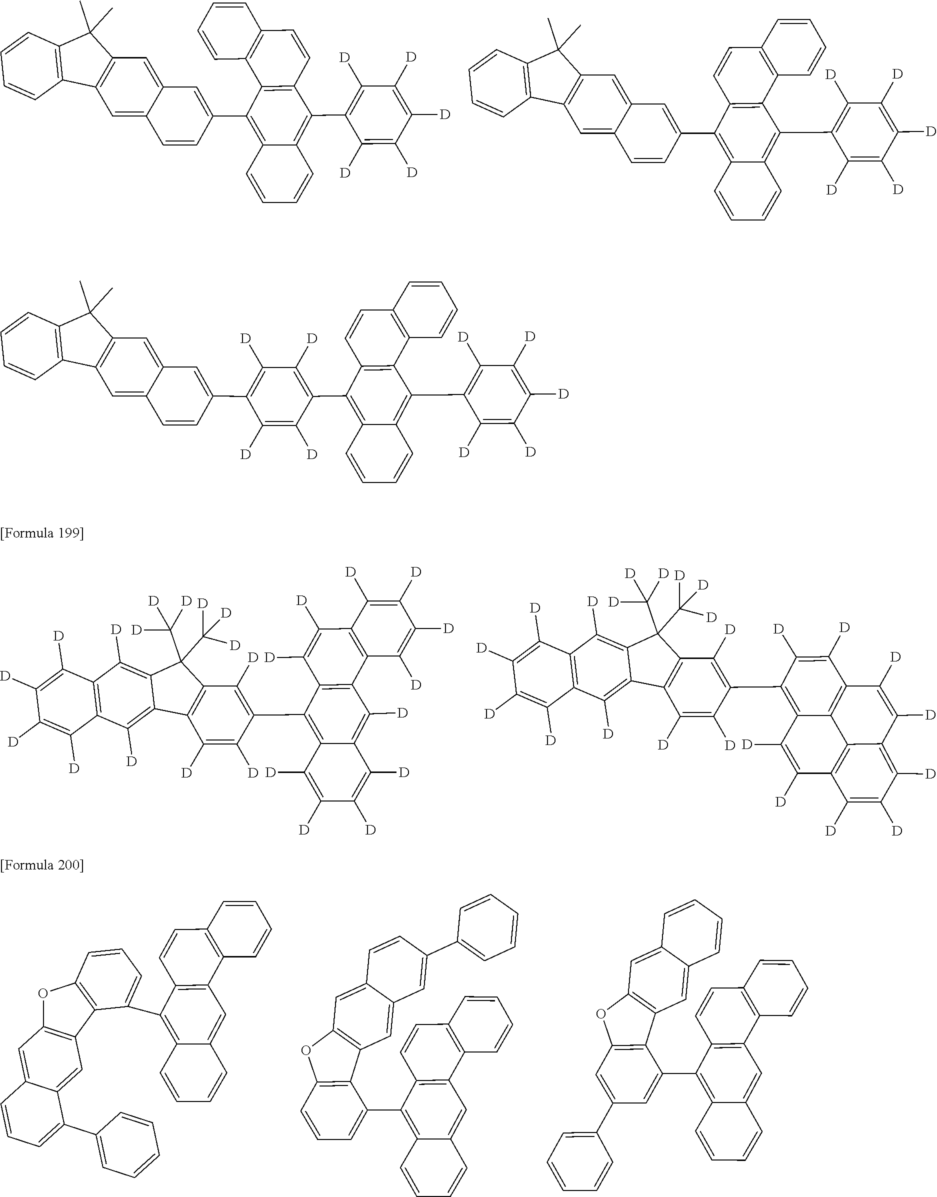

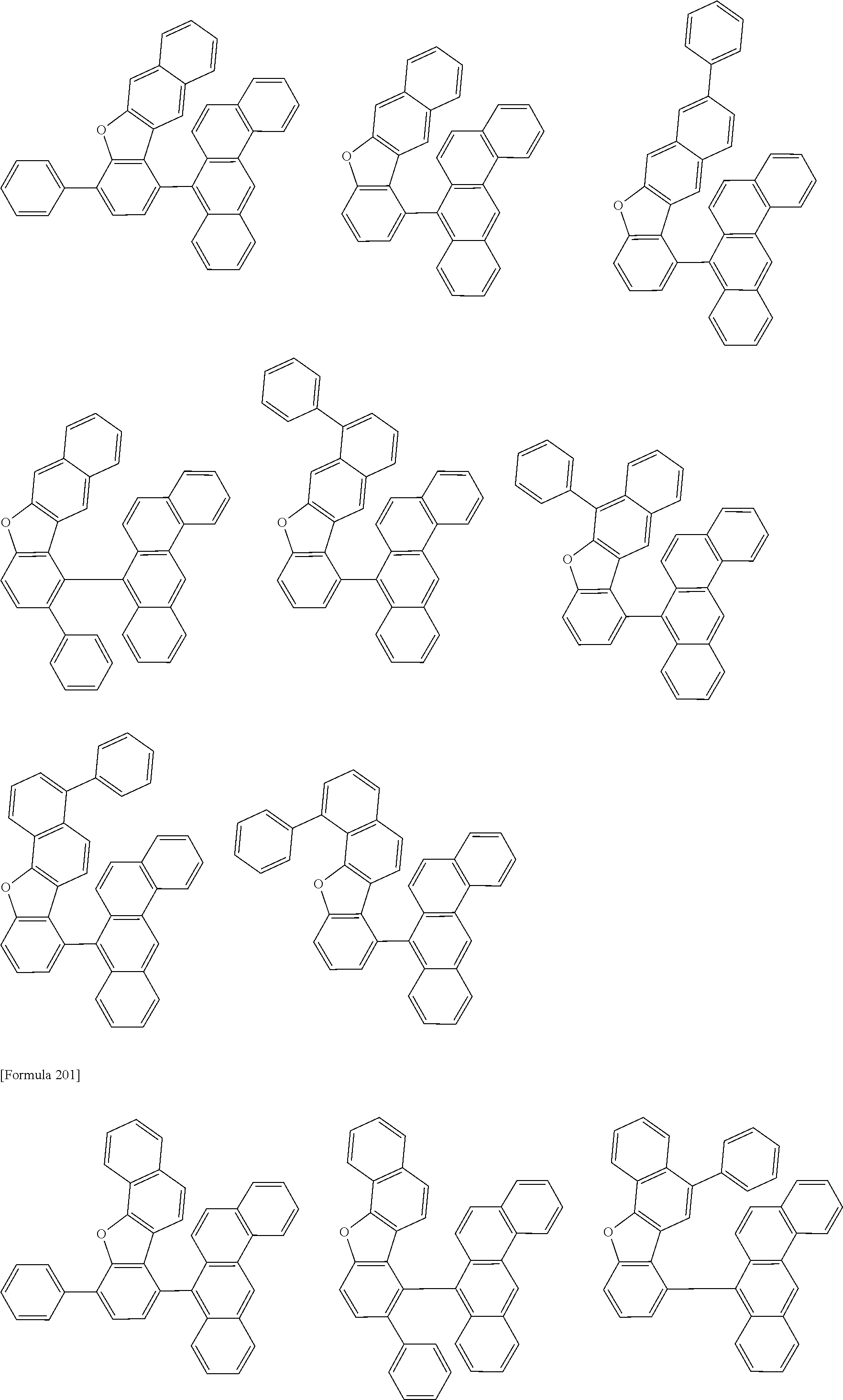

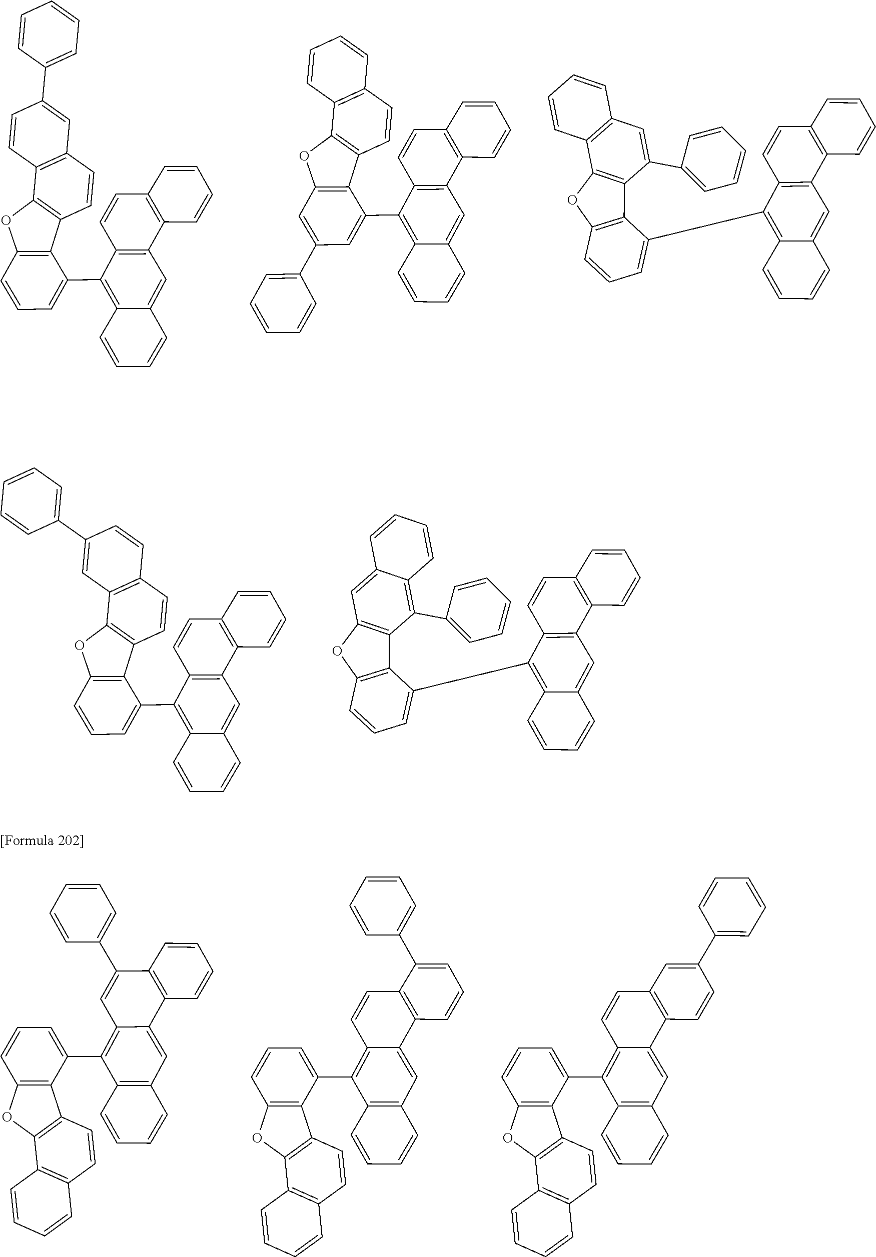

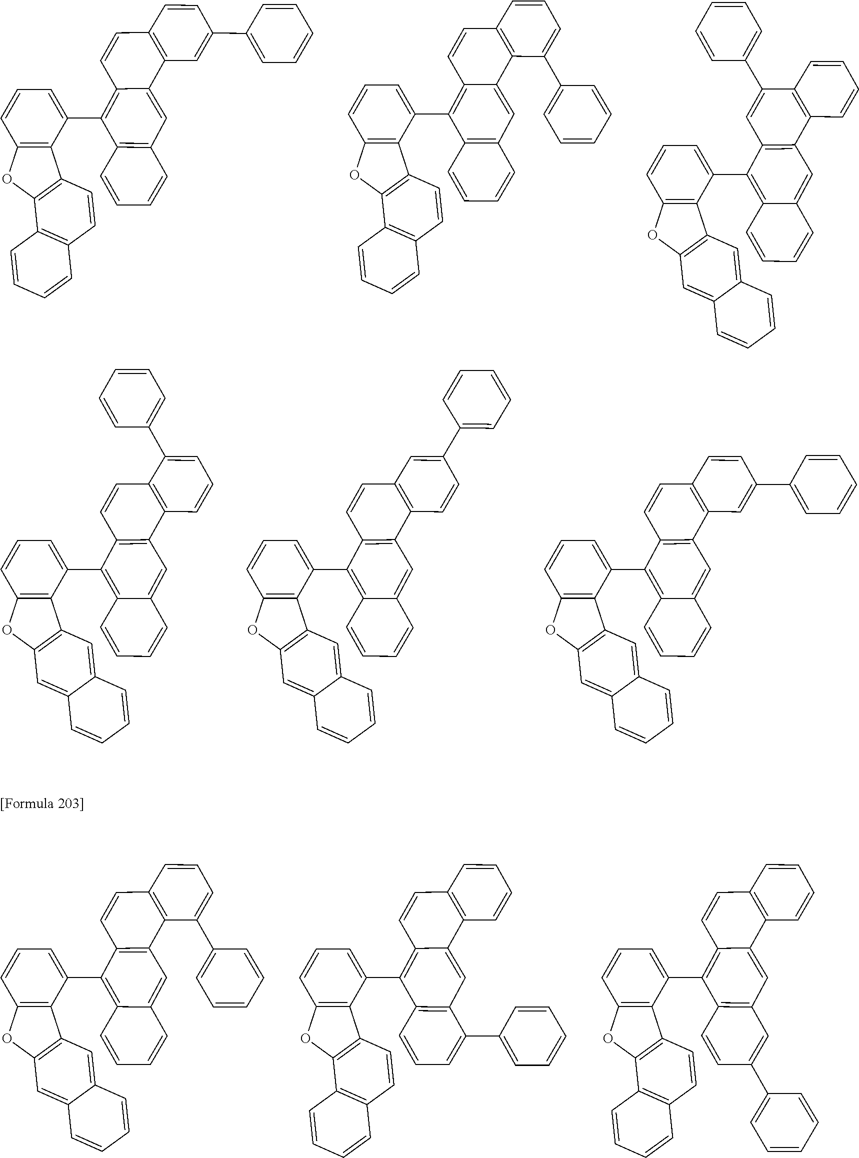

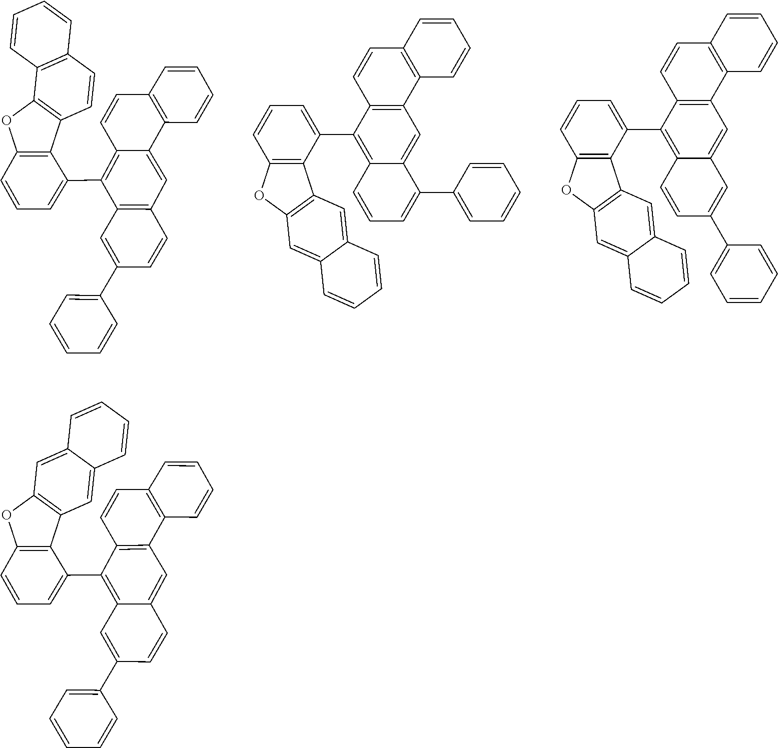 [SMILES: c1ccc(-c2ccc3cc4c(ccc5ccccc54)c(-c4cccc5oc6c7ccccc7ccc6c45)c3c2)cc1 ; c1ccc(-c2cccc3c(-c4cccc5oc6cc7ccccc7cc6c45)c4ccc5ccccc5c4cc23)cc1 ; c1ccc(-c2ccc3c(-c4cccc5oc6cc7ccccc7cc6c45)c4ccc5ccccc5c4cc3c2)cc1 ; c1ccc(-c2ccc3cc4c(ccc5ccccc54)c(-c4cccc5oc6cc7ccccc7cc6c45)c3c2)cc1]

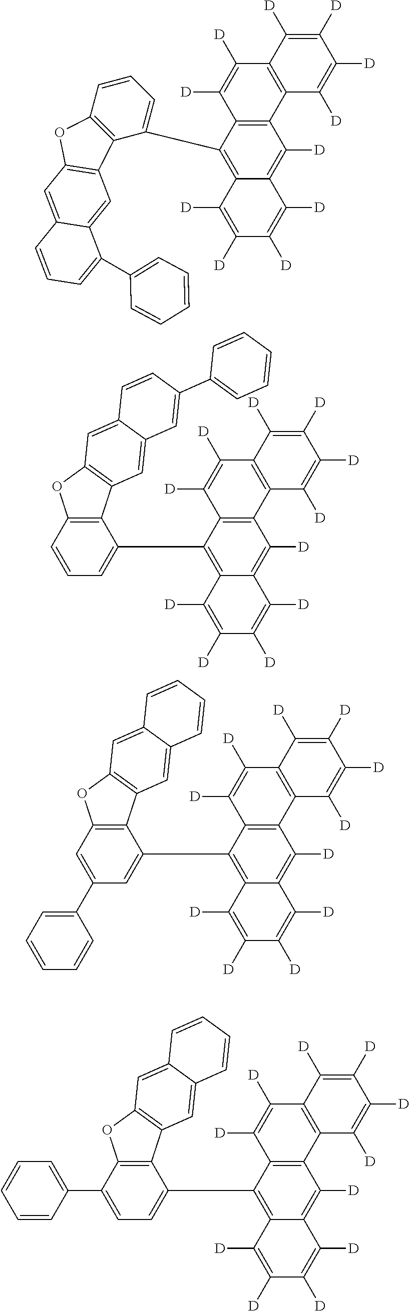

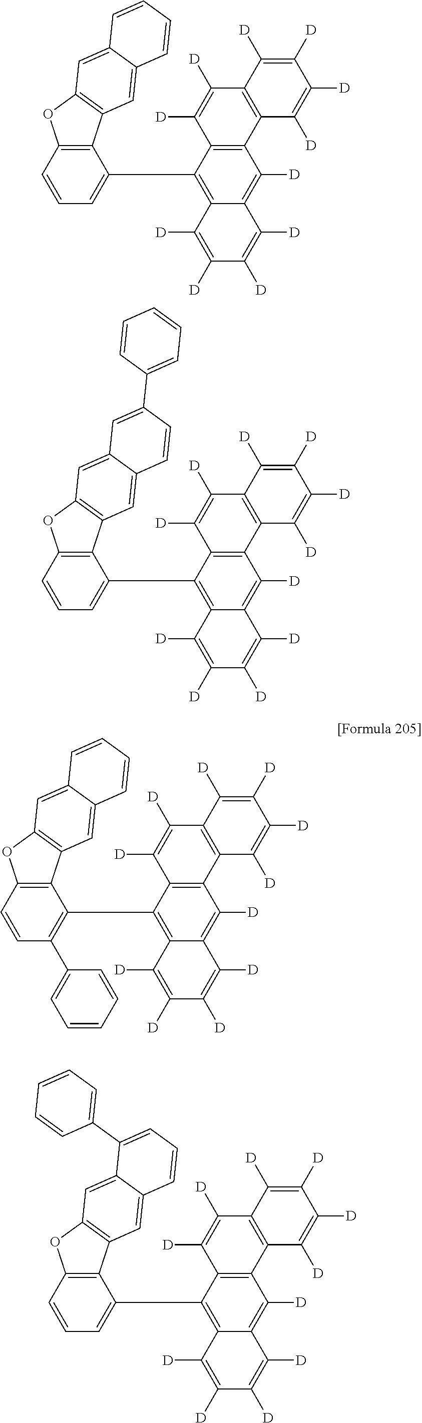

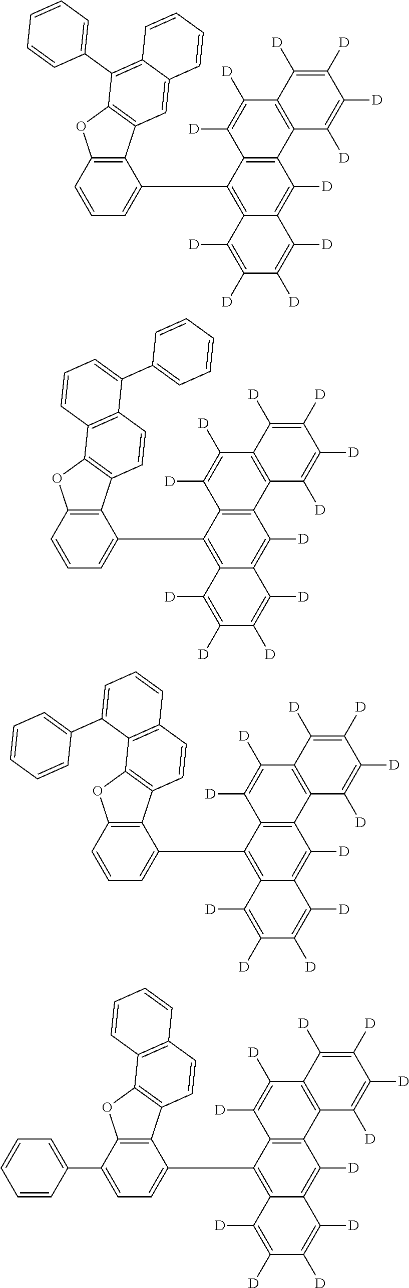

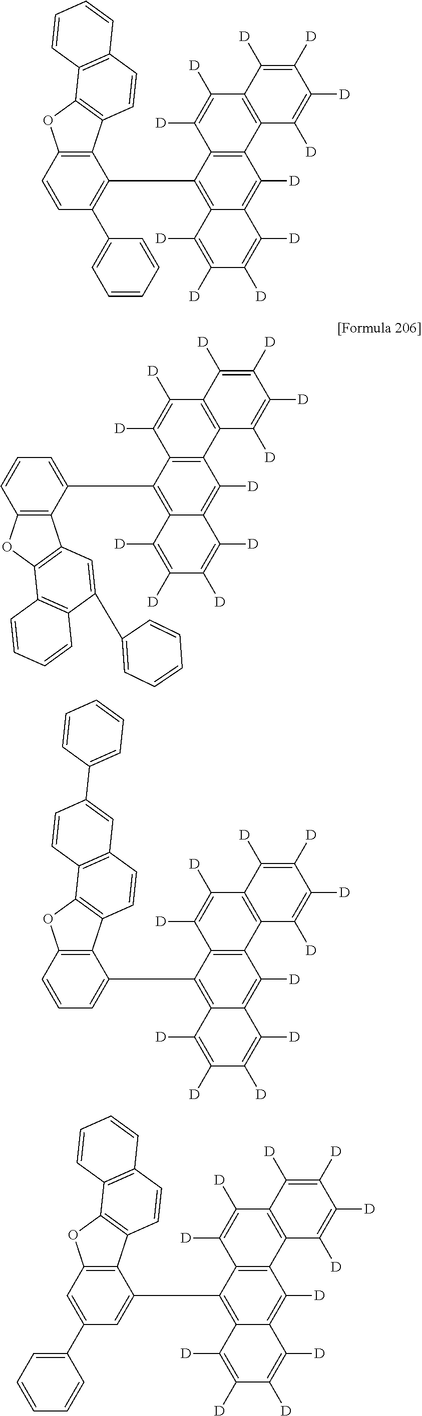

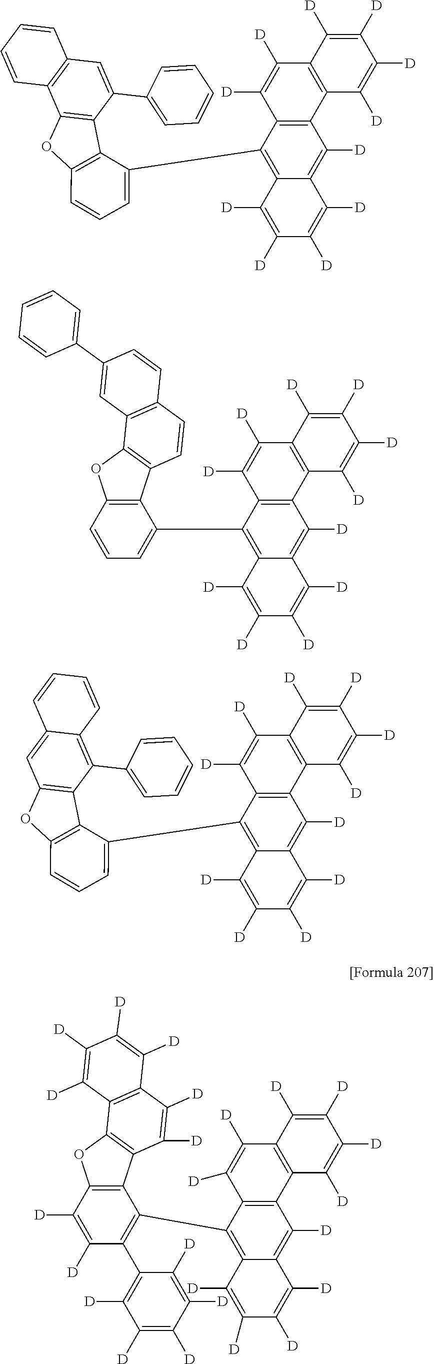

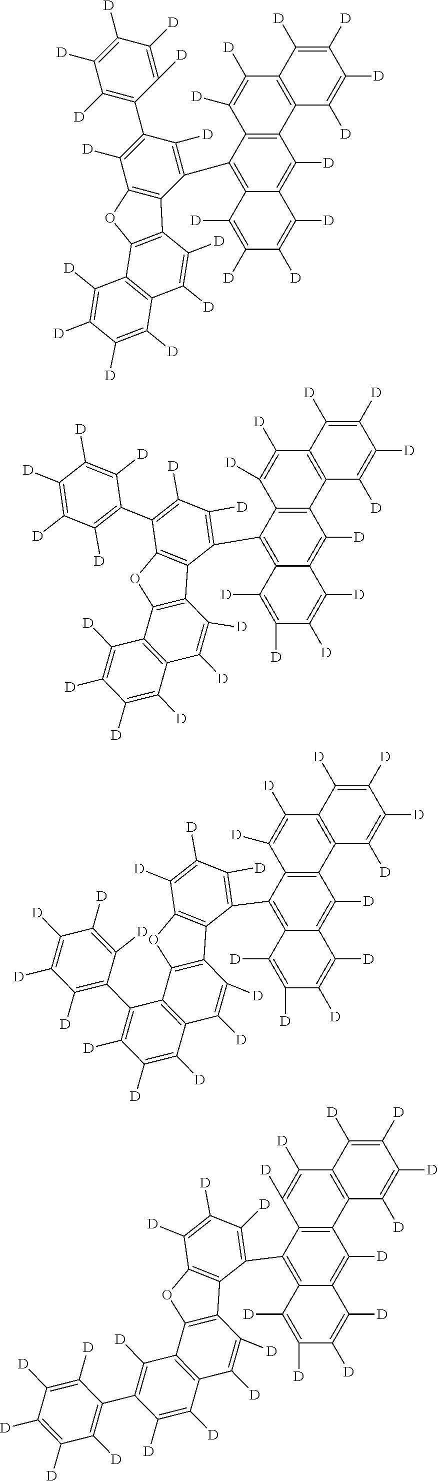

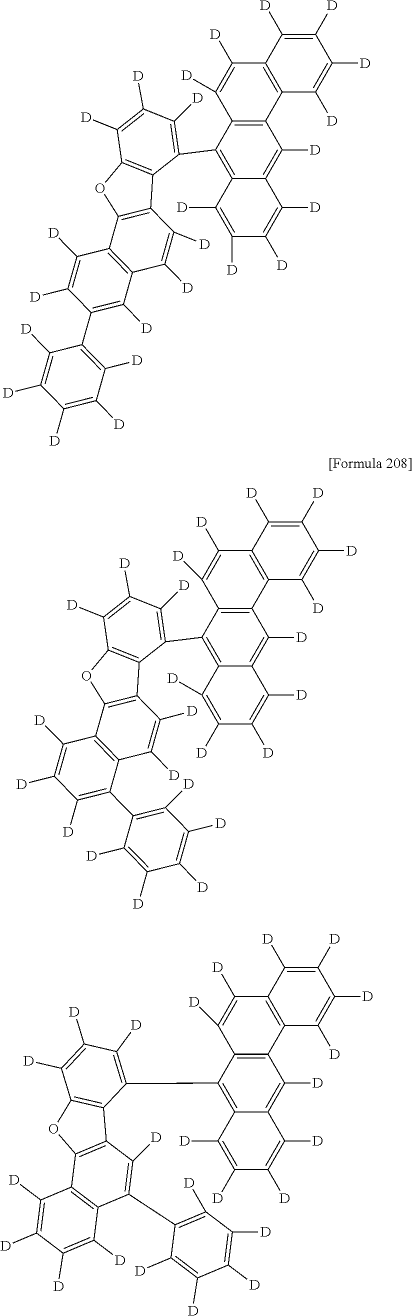

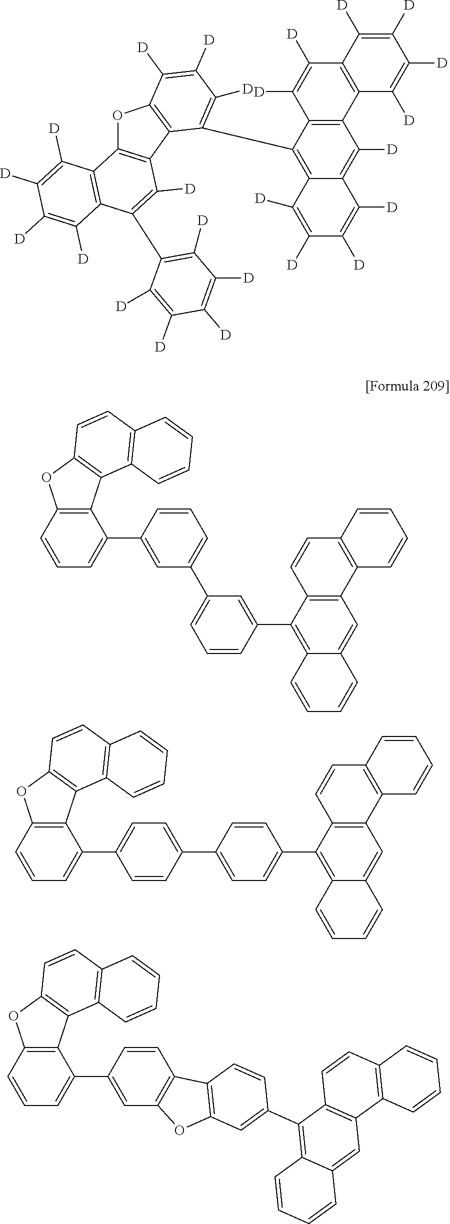 [SMILES: [2H]c1c([2H])c([2H])c(-c2c([2H])c3c(oc4c([2H])c([2H])c([2H])c(-c5c6c([2H])c([2H])c([2H])c([2H])c6c([2H])c6c5c([2H])c([2H])c5c([2H])c([2H])c([2H])c([2H])c56)c43)c3c([2H])c([2H])c([2H])c([2H])c23)c([2H])c1[2H] ; c1cc(-c2cccc(-c3cccc4oc5ccc6ccccc6c5c34)c2)cc(-c2c3ccccc3cc3c2ccc2ccccc23)c1 ; c1ccc2c(-c3ccc(-c4ccc(-c5cccc6oc7ccc8ccccc8c7c56)cc4)cc3)c3ccc4ccccc4c3cc2c1 ; c1ccc2c(-c3ccc4c(c3)oc3cc(-c5cccc6oc7ccc8ccccc8c7c56)ccc34)c3ccc4ccccc4c3cc2c1]

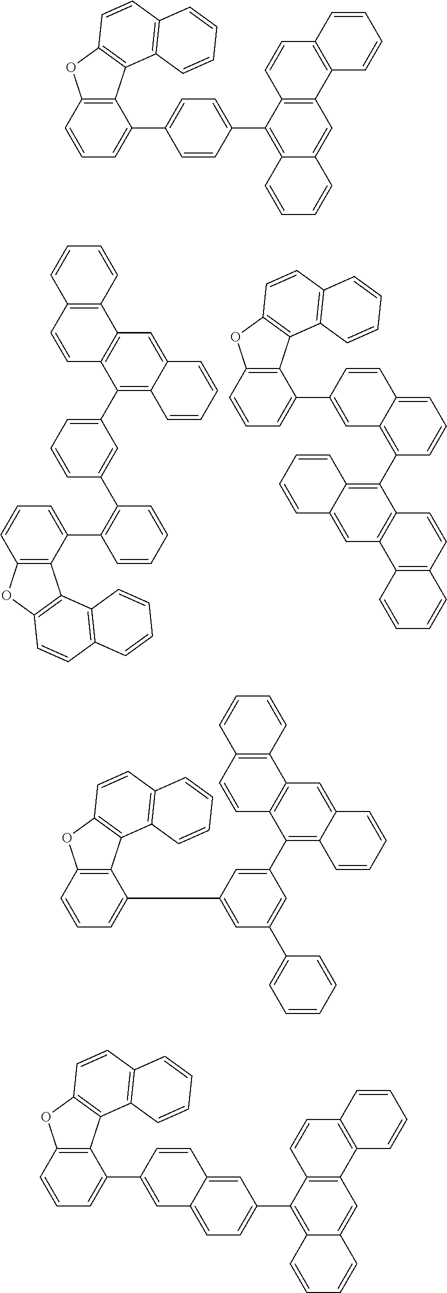

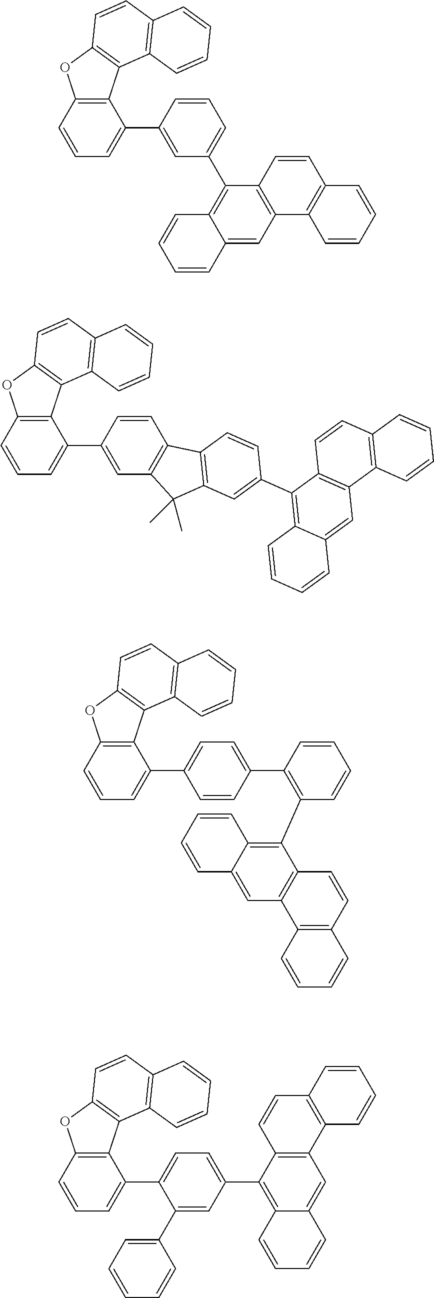 [SMILES: c1cc(-c2c3ccccc3cc3c2ccc2ccccc23)cc(-c2cccc3oc4ccc5ccccc5c4c23)c1 ; CC1(C)c2cc(-c3c4ccccc4cc4c3ccc3ccccc34)ccc2-c2ccc(-c3cccc4oc5ccc6ccccc6c5c34)cc21 ; c1ccc(-c2c3ccccc3cc3c2ccc2ccccc23)c(-c2ccc(-c3cccc4oc5ccc6ccccc6c5c34)cc2)c1 ; c1ccc(-c2cc(-c3c4ccccc4cc4c3ccc3ccccc34)ccc2-c2cccc3oc4ccc5ccccc5c4c23)cc1]

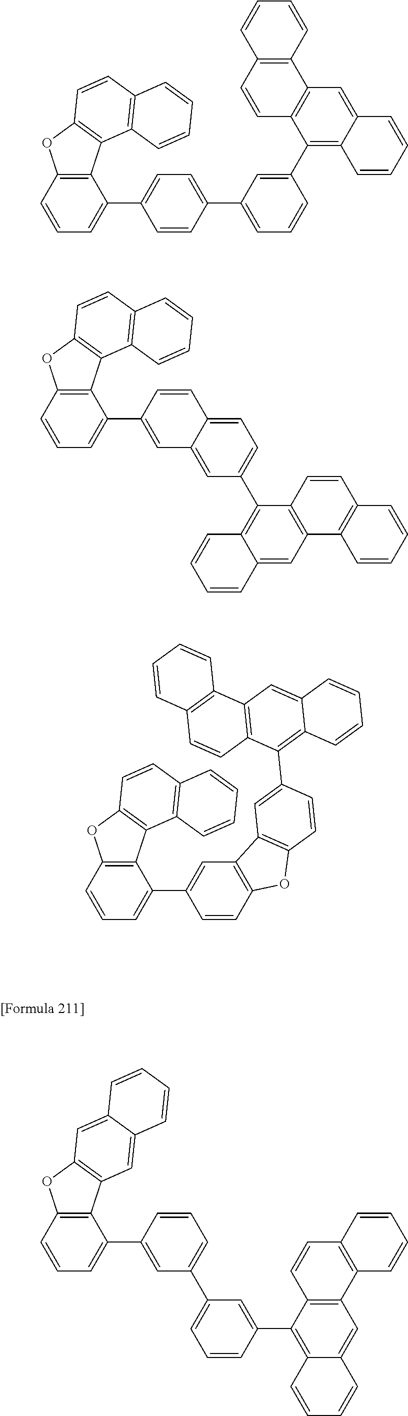 [SMILES: c1cc(-c2ccc(-c3cccc4oc5ccc6ccccc6c5c34)cc2)cc(-c2c3ccccc3cc3c2ccc2ccccc23)c1 ; c1ccc2c(-c3ccc4ccc(-c5cccc6oc7ccc8ccccc8c7c56)cc4c3)c3ccc4ccccc4c3cc2c1 ; c1ccc2c(-c3ccc4oc5ccc(-c6cccc7oc8ccc9ccccc9c8c67)cc5c4c3)c3ccc4ccccc4c3cc2c1 ; c1cc(-c2cccc(-c3cccc4oc5cc6ccccc6cc5c34)c2)cc(-c2c3ccccc3cc3c2ccc2ccccc23)c1]

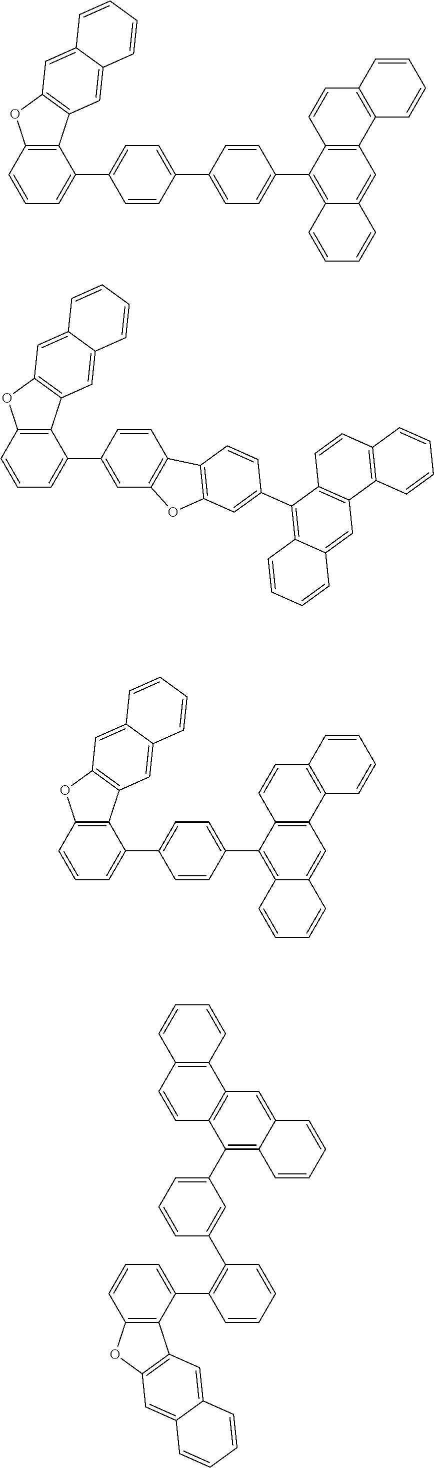 [SMILES: c1ccc2cc3c(cc2c1)oc1cccc(-c2ccc(-c4ccc(-c5c6ccccc6cc6c5ccc5ccccc56)cc4)cc2)c13 ; c1ccc2cc3c(cc2c1)oc1cccc(-c2ccc4c(c2)oc2cc(-c5c6ccccc6cc6c5ccc5ccccc56)ccc24)c13 ; c1ccc2cc3c(cc2c1)oc1cccc(-c2ccc(-c4c5ccccc5cc5c4ccc4ccccc45)cc2)c13 ; c1cc(-c2ccccc2-c2cccc3oc4cc5ccccc5cc4c23)cc(-c2c3ccccc3cc3c2ccc2ccccc23)c1]

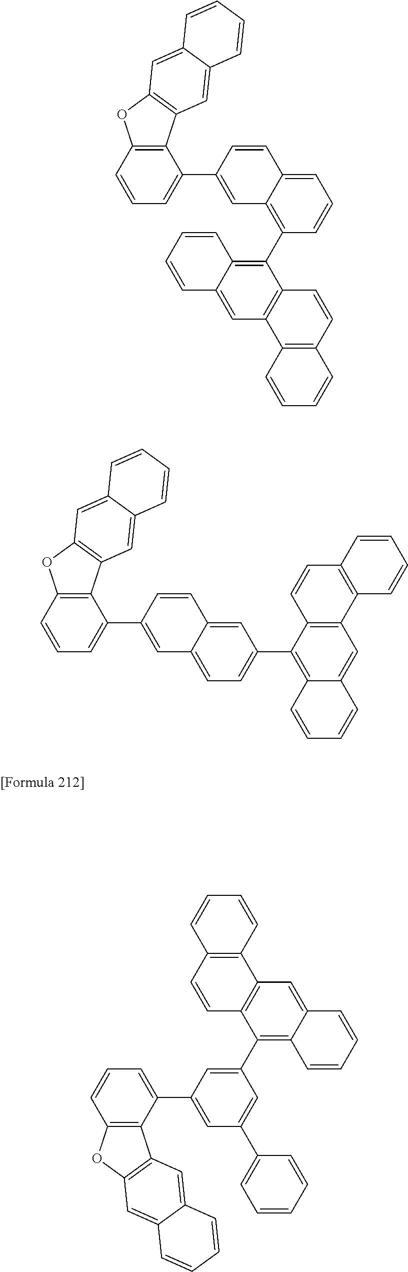 [SMILES: c1ccc2cc3c(cc2c1)oc1cccc(-c2ccc4cccc(-c5c6ccccc6cc6c5ccc5ccccc56)c4c2)c13 ; c1ccc2cc3c(cc2c1)oc1cccc(-c2ccc4cc(-c5c6ccccc6cc6c5ccc5ccccc56)ccc4c2)c13 ; c1ccc(-c2cc(-c3c4ccccc4cc4c3ccc3ccccc34)cc(-c3cccc4oc5cc6ccccc6cc5c34)c2)cc1]

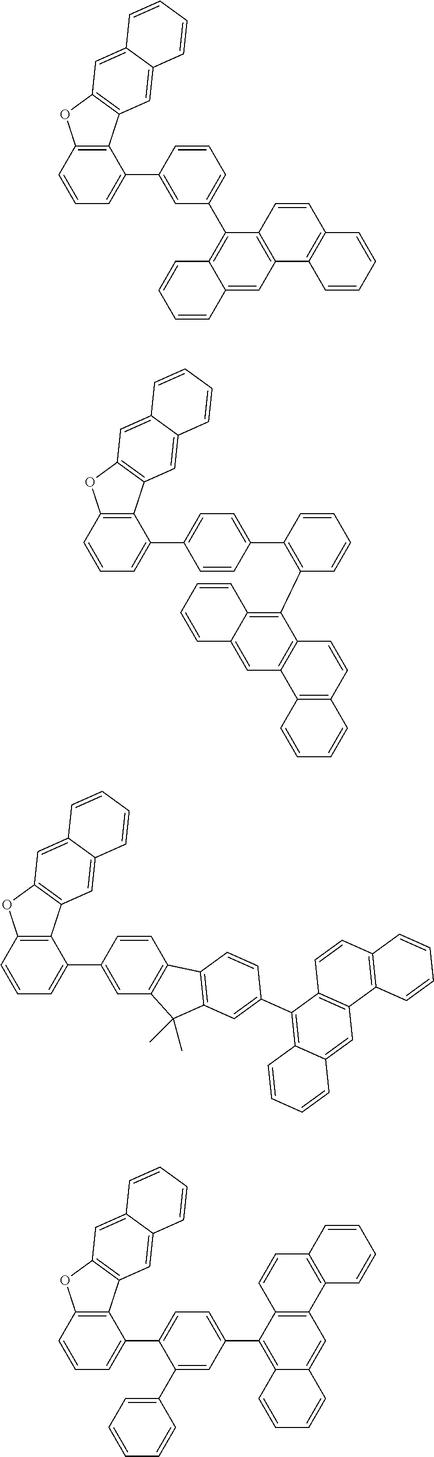 [SMILES: c1cc(-c2c3ccccc3cc3c2ccc2ccccc23)cc(-c2cccc3oc4cc5ccccc5cc4c23)c1 ; c1ccc(-c2c3ccccc3cc3c2ccc2ccccc23)c(-c2ccc(-c3cccc4oc5cc6ccccc6cc5c34)cc2)c1 ; CC1(C)c2cc(-c3c4ccccc4cc4c3ccc3ccccc34)ccc2-c2ccc(-c3cccc4oc5cc6ccccc6cc5c34)cc21 ; c1ccc(-c2cc(-c3c4ccccc4cc4c3ccc3ccccc34)ccc2-c2cccc3oc4cc5ccccc5cc4c23)cc1]

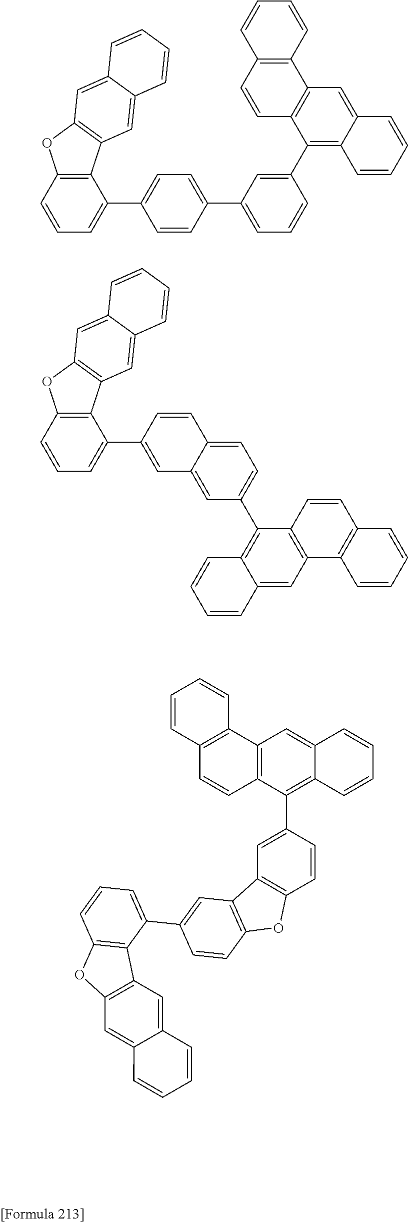

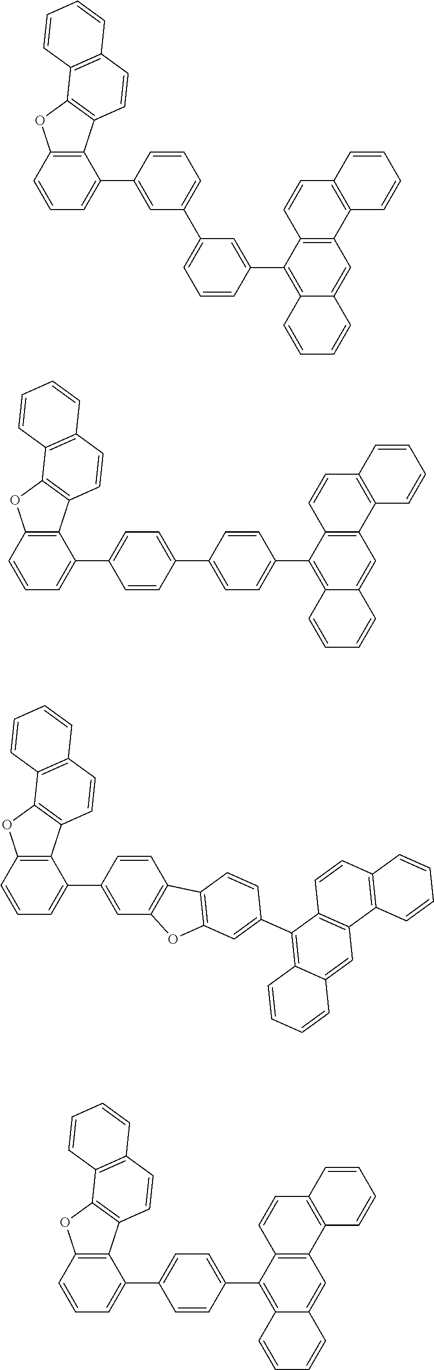 [SMILES: c1cc(-c2cccc(-c3cccc4oc5c6ccccc6ccc5c34)c2)cc(-c2c3ccccc3cc3c2ccc2ccccc23)c1 ; c1ccc2c(-c3ccc(-c4ccc(-c5cccc6oc7c8ccccc8ccc7c56)cc4)cc3)c3ccc4ccccc4c3cc2c1 ; c1ccc2c(-c3ccc4c(c3)oc3cc(-c5cccc6oc7c8ccccc8ccc7c56)ccc34)c3ccc4ccccc4c3cc2c1 ; c1ccc2c(-c3ccc(-c4cccc5oc6c7ccccc7ccc6c45)cc3)c3ccc4ccccc4c3cc2c1]

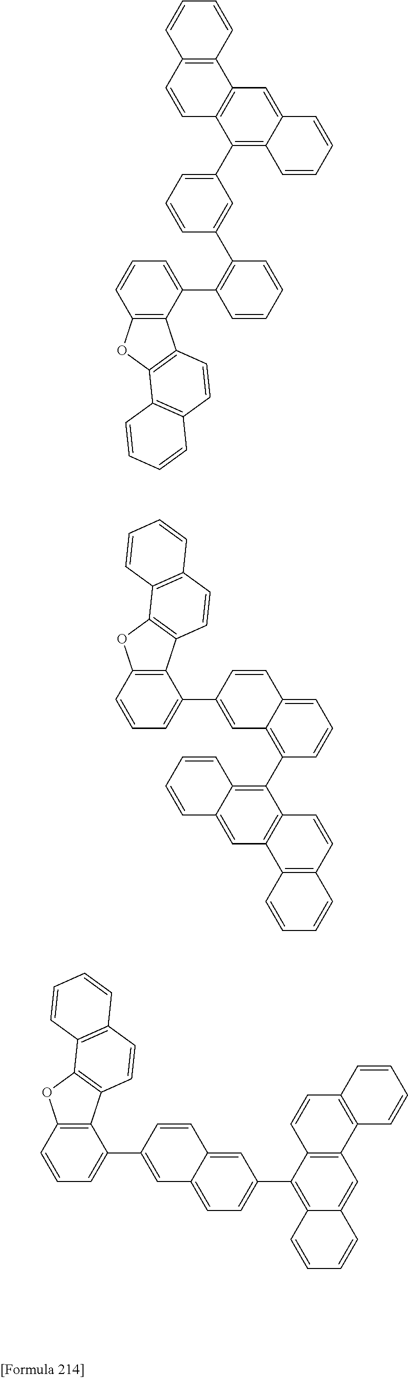 [SMILES: c1cc(-c2ccccc2-c2cccc3oc4c5ccccc5ccc4c23)cc(-c2c3ccccc3cc3c2ccc2ccccc23)c1 ; c1cc(-c2c3ccccc3cc3c2ccc2ccccc23)c2cc(-c3cccc4oc5c6ccccc6ccc5c34)ccc2c1 ; c1ccc2c(-c3ccc4cc(-c5cccc6oc7c8ccccc8ccc7c56)ccc4c3)c3ccc4ccccc4c3cc2c1]

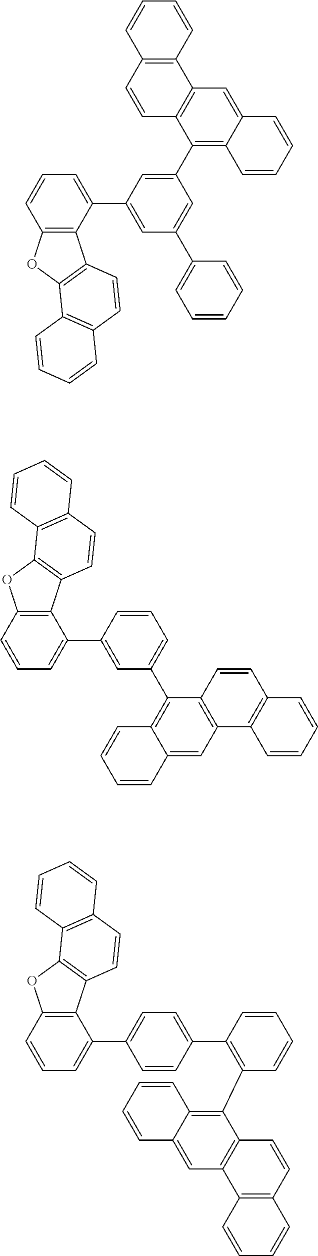 [SMILES: c1ccc(-c2cc(-c3c4ccccc4cc4c3ccc3ccccc34)cc(-c3cccc4oc5c6ccccc6ccc5c34)c2)cc1 ; c1cc(-c2c3ccccc3cc3c2ccc2ccccc23)cc(-c2cccc3oc4c5ccccc5ccc4c23)c1 ; c1ccc(-c2c3ccccc3cc3c2ccc2ccccc23)c(-c2ccc(-c3cccc4oc5c6ccccc6ccc5c34)cc2)c1]

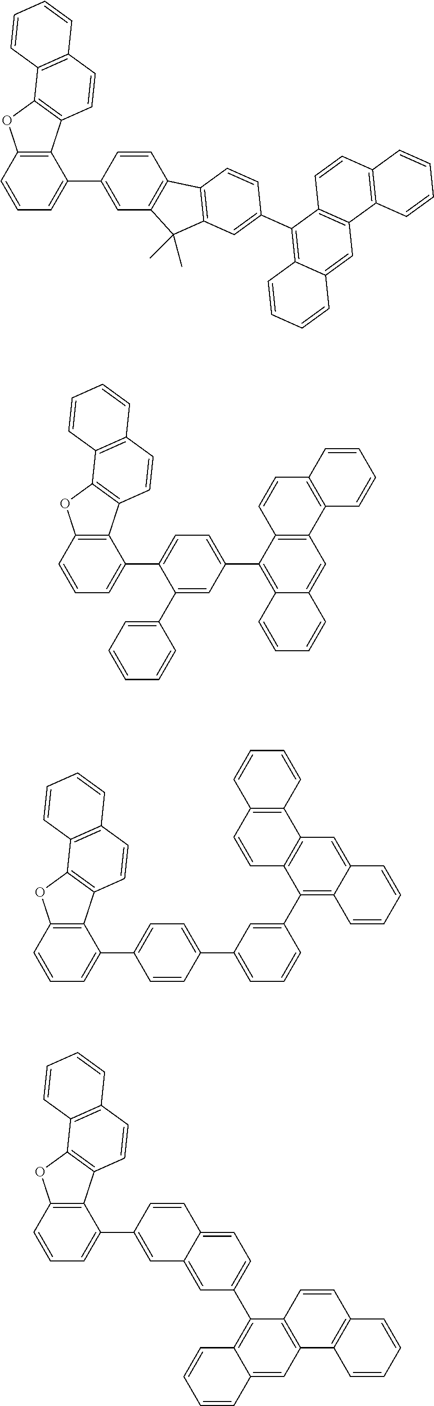 [SMILES: CC1(C)c2cc(-c3c4ccccc4cc4c3ccc3ccccc34)ccc2-c2ccc(-c3cccc4oc5c6ccccc6ccc5c34)cc21 ; c1ccc(-c2cc(-c3c4ccccc4cc4c3ccc3ccccc34)ccc2-c2cccc3oc4c5ccccc5ccc4c23)cc1 ; c1cc(-c2ccc(-c3cccc4oc5c6ccccc6ccc5c34)cc2)cc(-c2c3ccccc3cc3c2ccc2ccccc23)c1 ; c1ccc2c(-c3ccc4ccc(-c5cccc6oc7c8ccccc8ccc7c56)cc4c3)c3ccc4ccccc4c3cc2c1]

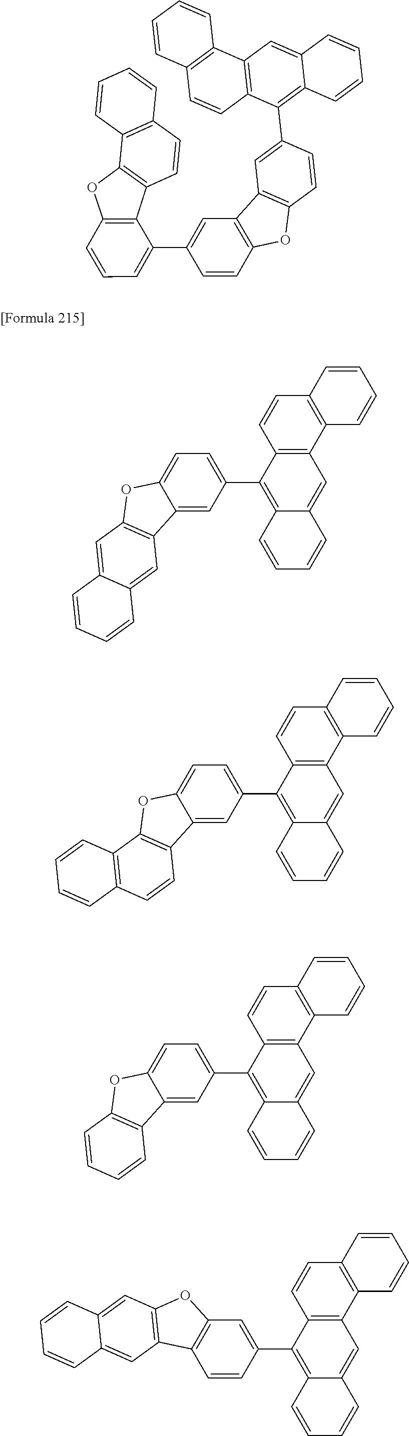 [SMILES: c1ccc2c(-c3ccc4oc5ccc(-c6cccc7oc8c9ccccc9ccc8c67)cc5c4c3)c3ccc4ccccc4c3cc2c1 ; c1ccc2cc3c(cc2c1)oc1ccc(-c2c4ccccc4cc4c2ccc2ccccc24)cc13 ; c1ccc2c(-c3ccc4oc5c6ccccc6ccc5c4c3)c3ccc4ccccc4c3cc2c1 ; c1ccc2c(-c3ccc4oc5ccccc5c4c3)c3ccc4ccccc4c3cc2c1 ; c1ccc2cc3c(cc2c1)oc1cc(-c2c4ccccc4cc4c2ccc2ccccc24)ccc13]

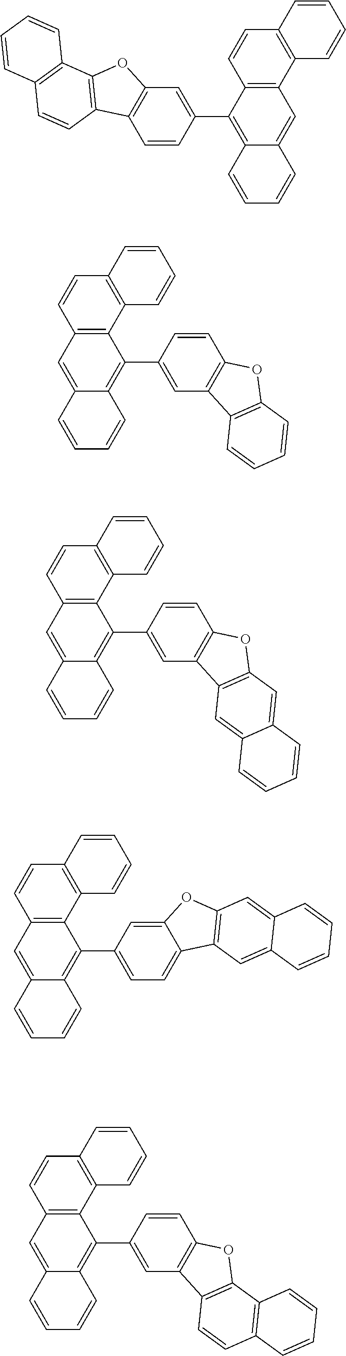 [SMILES: c1ccc2c(-c3ccc4c(c3)oc3c5ccccc5ccc43)c3ccc4ccccc4c3cc2c1 ; c1ccc2c(-c3ccc4oc5ccccc5c4c3)c3c(ccc4ccccc43)cc2c1 ; c1ccc2cc3c(cc2c1)oc1ccc(-c2c4ccccc4cc4ccc5ccccc5c24)cc13 ; c1ccc2cc3c(cc2c1)oc1cc(-c2c4ccccc4cc4ccc5ccccc5c24)ccc13 ; c1ccc2c(-c3ccc4oc5c6ccccc6ccc5c4c3)c3c(ccc4ccccc43)cc2c1]

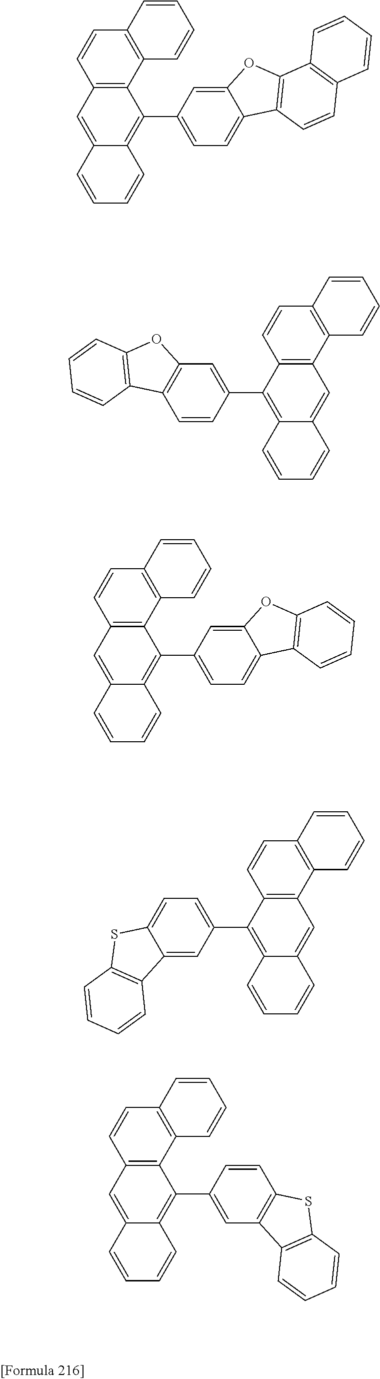 [SMILES: c1ccc2c(-c3ccc4c(c3)oc3c5ccccc5ccc43)c3c(ccc4ccccc43)cc2c1 ; c1ccc2c(-c3ccc4c(c3)oc3ccccc34)c3ccc4ccccc4c3cc2c1 ; c1ccc2c(-c3ccc4c(c3)oc3ccccc34)c3c(ccc4ccccc43)cc2c1 ; c1ccc2c(-c3ccc4sc5ccccc5c4c3)c3ccc4ccccc4c3cc2c1 ; c1ccc2c(-c3ccc4sc5ccccc5c4c3)c3c(ccc4ccccc43)cc2c1]

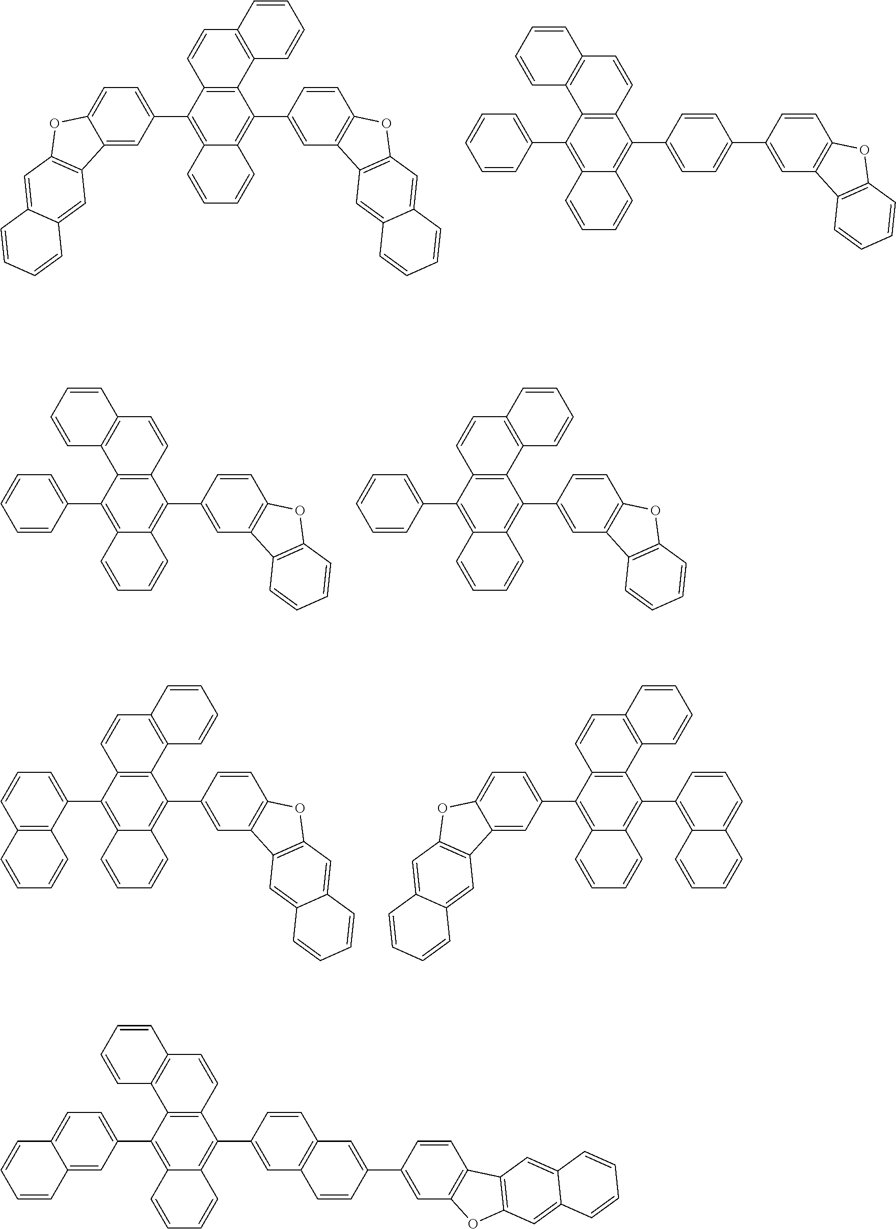

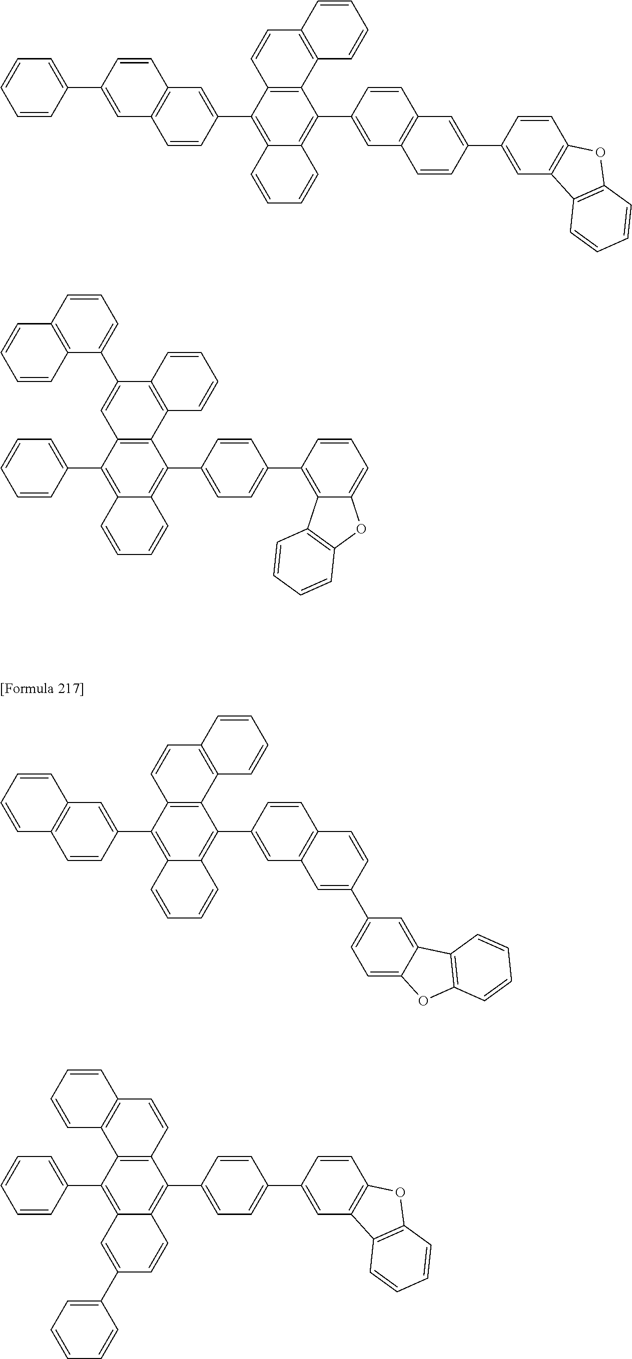 [SMILES: c1ccc(-c2ccc3cc(-c4c5ccccc5c(-c5ccc6cc(-c7ccc8oc9ccccc9c8c7)ccc6c5)c5c4ccc4ccccc45)ccc3c2)cc1 ; c1ccc(-c2c3ccccc3c(-c3ccc(-c4cccc5oc6ccccc6c45)cc3)c3c2cc(-c2cccc4ccccc24)c2ccccc23)cc1 ; c1ccc2cc(-c3c4ccccc4c(-c4ccc5ccc(-c6ccc7oc8ccccc8c7c6)cc5c4)c4c3ccc3ccccc34)ccc2c1 ; c1ccc(-c2ccc3c(-c4ccc(-c5ccc6oc7ccccc7c6c5)cc4)c4ccc5ccccc5c4c(-c4ccccc4)c3c2)cc1]

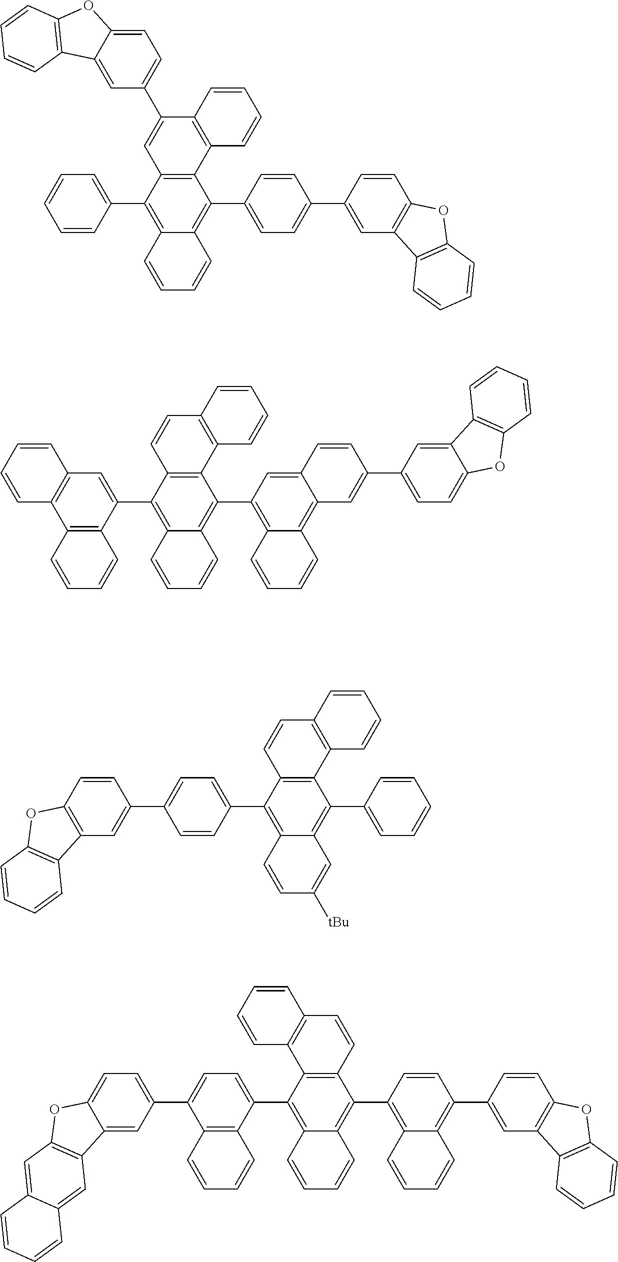 [SMILES: c1ccc(-c2c3ccccc3c(-c3ccc(-c4ccc5oc6ccccc6c5c4)cc3)c3c2cc(-c2ccc4oc5ccccc5c4c2)c2ccccc23)cc1 ; c1ccc2c(c1)cc(-c1c3ccccc3c(-c3cc4ccc(-c5ccc6oc7ccccc7c6c5)cc4c4ccccc34)c3c1ccc1ccccc13)c1ccccc12 ; CC(C)(C)c1ccc2c(-c3ccc(-c4ccc5oc6ccccc6c5c4)cc3)c3ccc4ccccc4c3c(-c3ccccc3)c2c1 ; c1ccc2cc3c(cc2c1)oc1ccc(-c2ccc(-c4c5ccccc5c(-c5ccc(-c6ccc7oc8ccccc8c7c6)c6ccccc56)c5ccc6ccccc6c45)c4ccccc24)cc13]

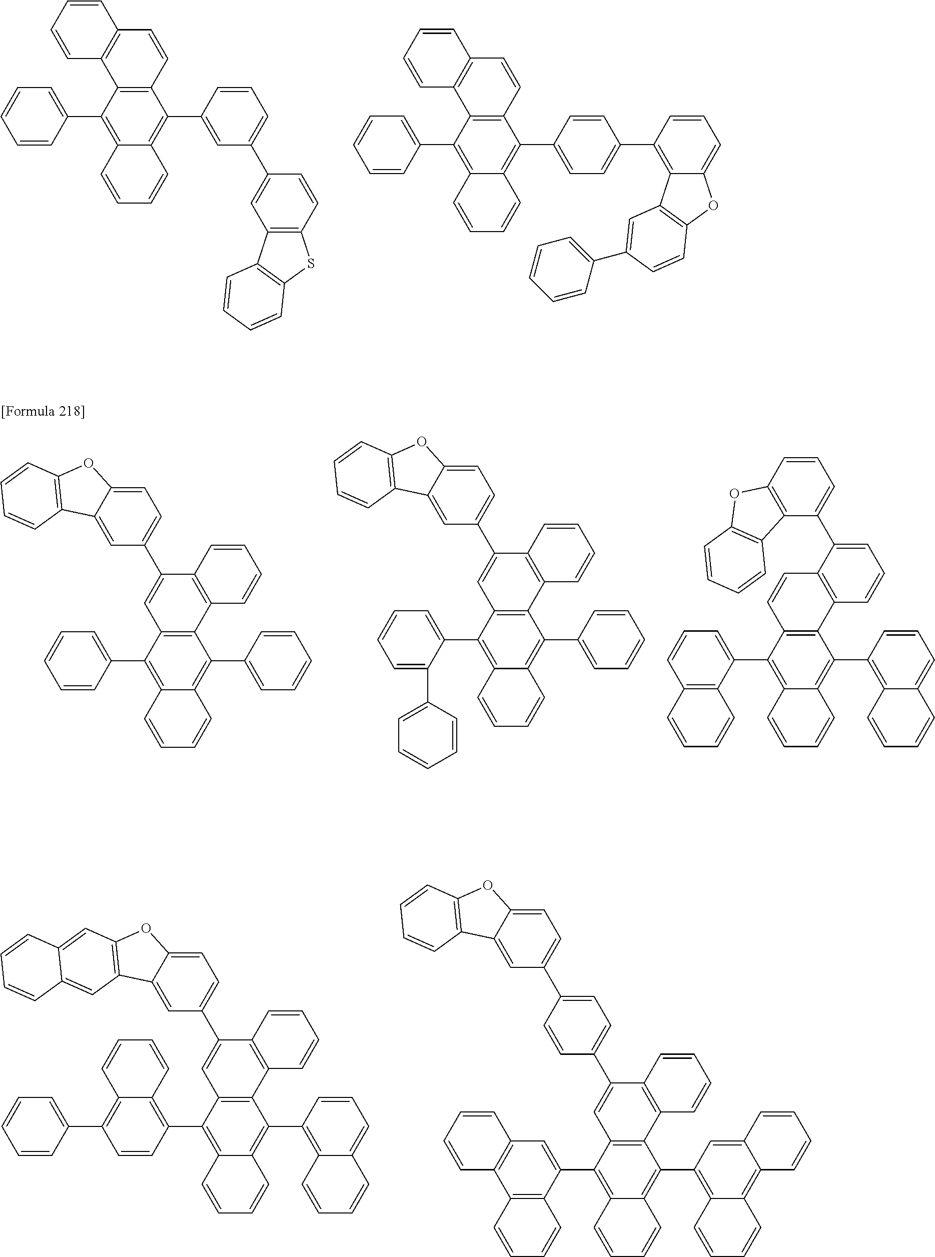

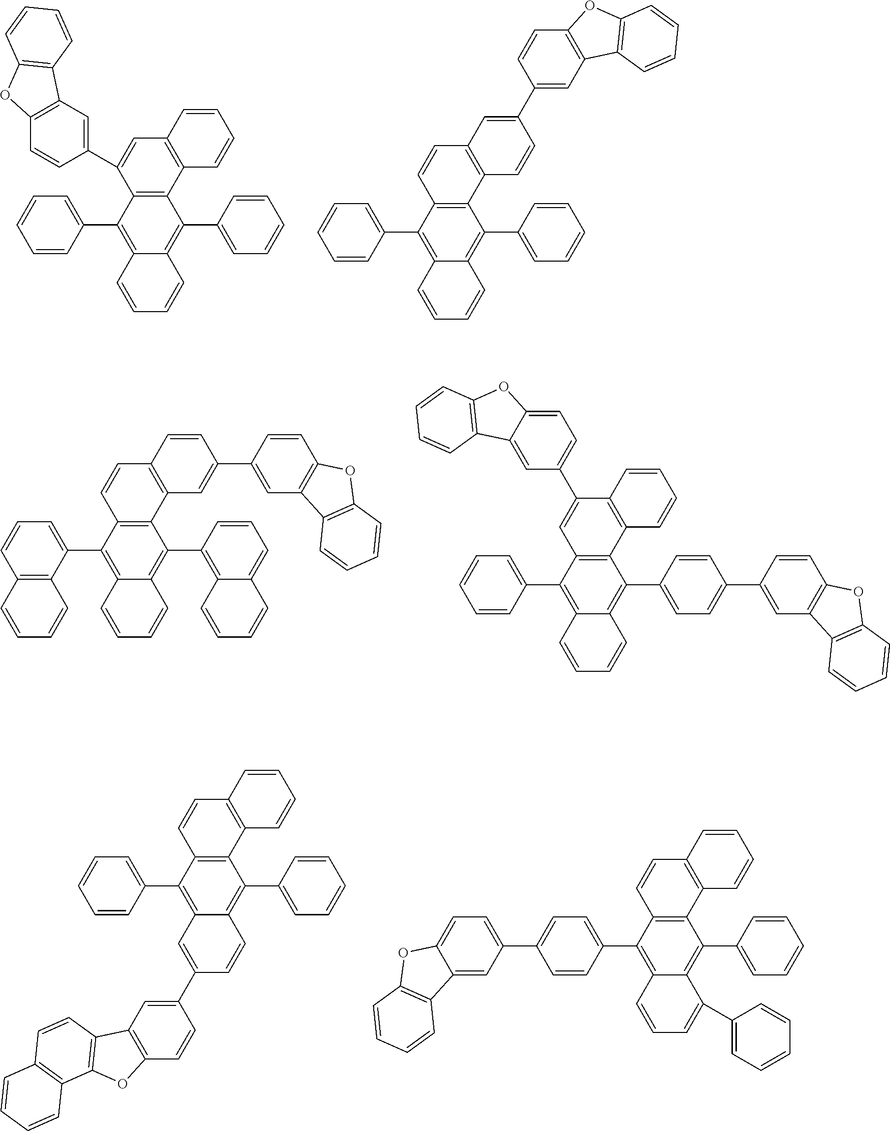

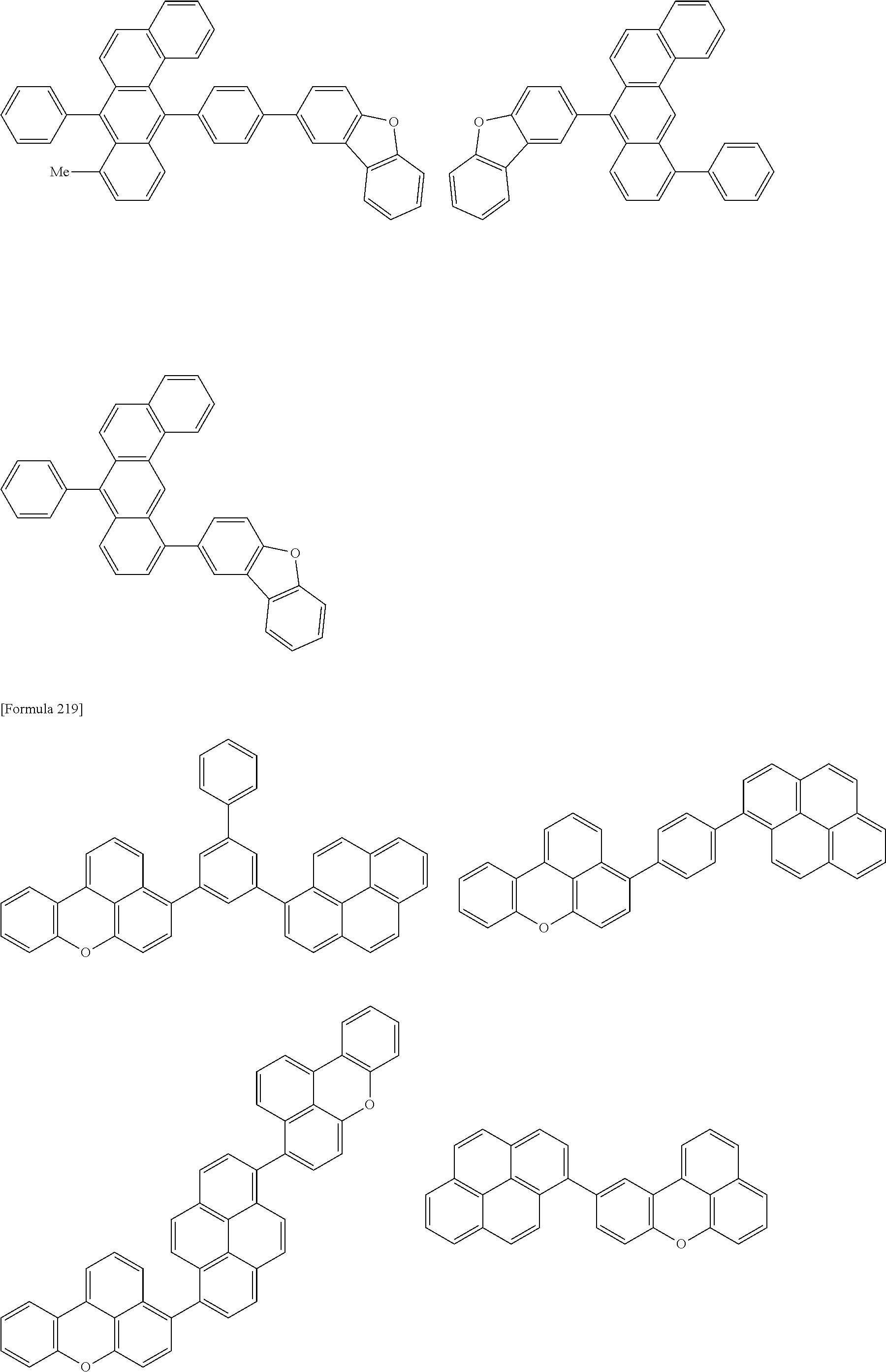

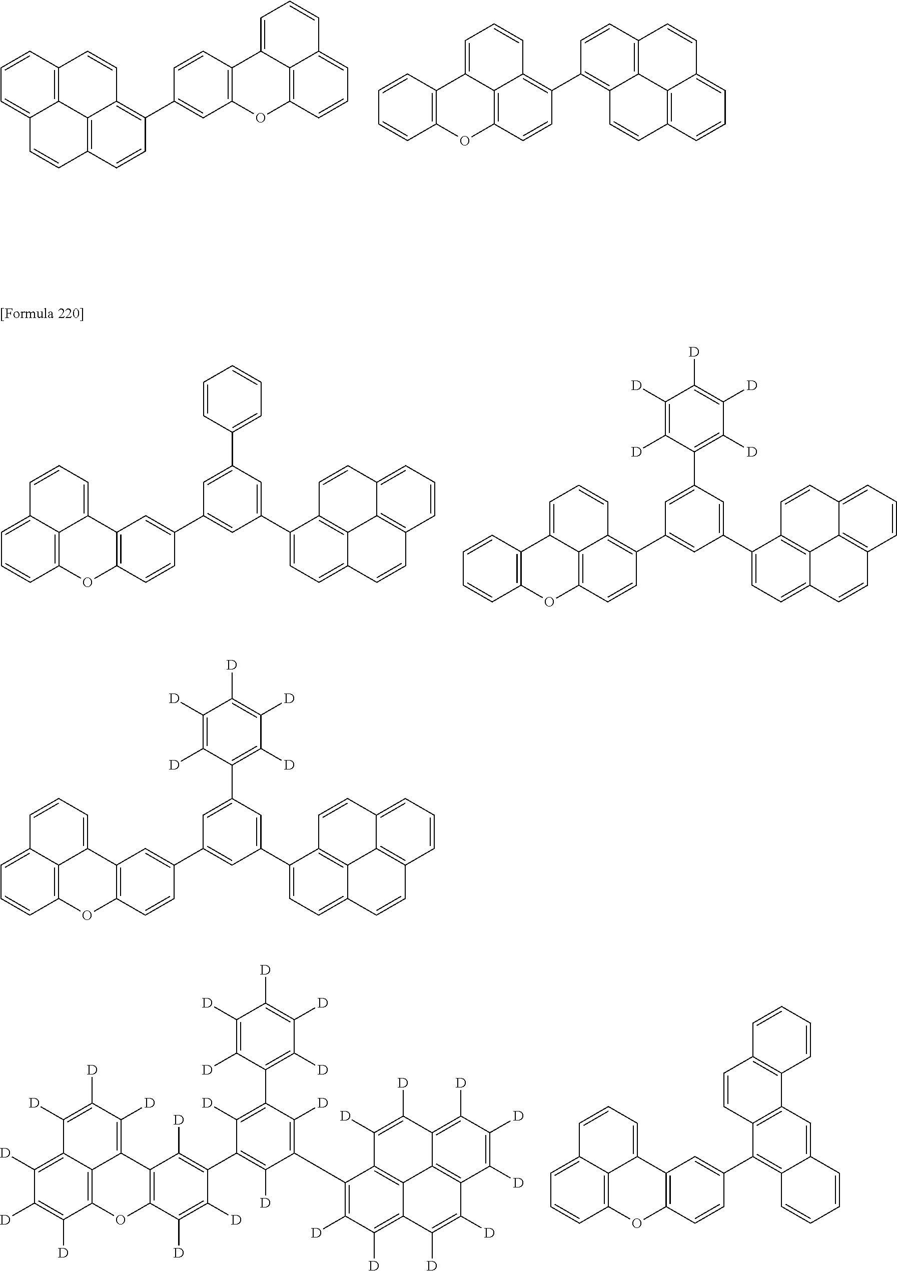

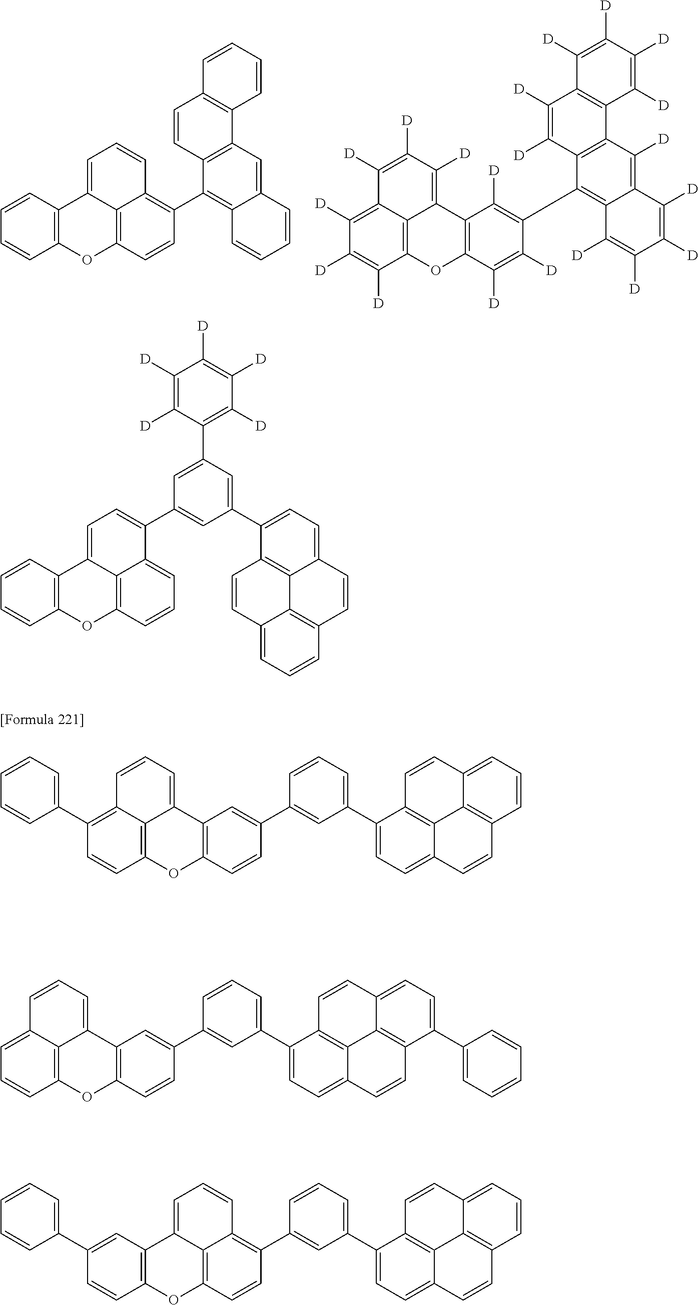

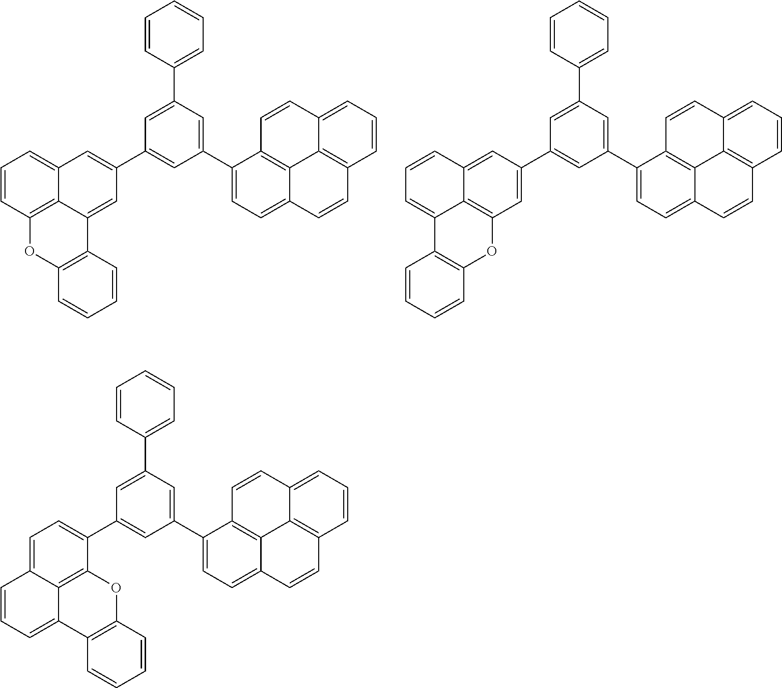

### Second Emitting Layer

The second emitting layer contains the second host material and the second luminescent compound that emits light having a maximum peak wavelength of 500 nm or less.

In an exemplary arrangement of the organic EL device of the exemplary embodiment, the second luminescent compound emits light having a maximum peak wavelength of 480 nm or less.

In an exemplary arrangement of the organic EL device of the exemplary embodiment, the second luminescent compound emits light having a maximum peak wavelength of 430 nm or more.

In an exemplary arrangement of the organic EL device of the exemplary embodiment, the second luminescent compound emits fluorescence having a maximum peak wavelength of 500 nm or less, or emits fluorescence having a maximum peak wavelength of 480 nm or less.

In an exemplary arrangement of the organic EL device of the exemplary embodiment, the second luminescent compound emits fluorescence having a maximum peak wavelength of 430 nm or more.

A method of measuring the maximum peak wavelength of the compound is as described above.

In an exemplary arrangement of the organic EL device of the exemplary embodiment, the full width at half maximum of the maximum peak of the second luminescent compound is in a range from 1 nm to 20 nm.

In an exemplary arrangement of the organic EL device of the exemplary embodiment, the second luminescent compound is a compound containing no azine ring structure in a molecule.

In an exemplary arrangement of the organic EL device of the exemplary embodiment, the second luminescent compound is not a boron-containing complex. In an exemplary arrangement of the organic EL device of the exemplary embodiment, the second luminescent compound is not a complex.

In an exemplary arrangement of the organic EL device of the exemplary embodiment, the second emitting layer contains no metal complex. In an exemplary arrangement of the organic EL device of the exemplary embodiment, the second emitting layer contains no boron-containing complex.

In an exemplary arrangement of the organic EL device of the exemplary embodiment, the second emitting layer contains no phosphorescent material (dopant material).

In an exemplary arrangement of the organic EL device of the exemplary embodiment, the second emitting layer contains neither a heavy-metal complex nor a phosphorescent rare earth metal complex. Examples of the heavy-metal complex herein include an iridium complex, osmium complex, and platinum complex.

In an exemplary arrangement of the organic EL device of the exemplary embodiment, a singlet energy of the second host material S₁(H2) and a singlet energy of the second luminescent compound S₁(D2) satisfy a relationship of a numerical formula (Numerical Formula 7) below.

$${S}_{1} ( H 2 ) > {S}_{1} ( D 2 ) \qquad ( Numerical Formula 7 )$$

When the second luminescent compound and the second host material satisfy the relationship of the numerical formula (Numerical Formula 7) in the organic EL device according to the exemplary embodiment, due to the singlet energy of the second luminescent compound being smaller than the singlet energy of the second host material, singlet excitons generated by the TTF phenomenon energy-transfer from the second host material to the second luminescent compound, contributing to the fluorescence of the second luminescent compound.

In an exemplary arrangement of the organic EL device of the exemplary embodiment, a triplet energy of the second luminescent compound T₁(D2) and the triplet energy of the second host material T₁(H2) satisfy a relationship of a numerical formula (Numerical Formula 8) below.

$${T}_{1} ( D 2 ) > {T}_{1} ( H 2 ) \qquad ( Numerical Formula 8 )$$

When the second luminescent compound and the second host material satisfy the relationship of the numerical formula (Numerical Formula 8) in the organic EL device according to the exemplary embodiment, in transfer of triplet excitons generated in the first emitting layer to the second emitting layer, the triplet excitons energy-transfer not onto the second luminescent compound having higher triplet energy but onto molecules of the second host material. In addition, triplet excitons generated by recombination of holes and electrons on the second host material do not transfer to the second luminescent compound having higher triplet energy. Triplet excitons generated by recombination on molecules of the second luminescent compound quickly energy-transfer to molecules of the second host material.

Triplet excitons in the second host material do not transfer to the second luminescent compound but efficiently collide with one another on the second host material to generate singlet excitons by the TTF phenomenon.

In an exemplary arrangement of the organic EL device of the exemplary embodiment, the triplet energy of the second luminescent compound T₁(D2) satisfies a relationship of a numerical formula (Numerical Formula 14C), (Numerical Formula 14D), or (Numerical Formula 14E) below.

$$2.7 eV > {T}_{1} ( D 2 ) \qquad ( Numerical Formula 14 C )$$

$$2.6 eV > {T}_{1} ( D 2 ) \qquad ( Numerical Formula 14 D )$$

$$2.5 eV > {T}_{1} ( D 2 ) \qquad ( Numerical Formula 14 E )$$

The organic EL device has a long lifetime when the second emitting layer contains a compound that satisfies the relationship of the numerical formula (Numerical Formula 14C), (Numerical Formula 14D), or (Numerical Formula 14E).

In an exemplary arrangement of the organic EL device of the exemplary embodiment, the triplet energy of the second host material T₁(H2) satisfies a relationship of a numerical formula (Numerical Formula 13) or (Numerical Formula 13A) below.

$${T}_{1} ( H 2 ) \geq 1.9 eV \qquad ( Numerical Formula 13 )$$

$${T}_{1} ( H 2 ) > 1.9 eV \qquad ( Numerical Formula 13 A )$$

In an exemplary arrangement of the organic EL device of the exemplary embodiment, the triplet energy of the second host material T₁(H2) satisfies a relationship of a numerical formula (Numerical Formula 13B) or (Numerical Formula 13C) below.

$$1.9 eV \geq {T}_{1} ( H 2 ) \qquad ( Numerical Formula 13 B )$$

$$1. 9 eV \geq {T}_{1} ( H 2 ) \geq 1.8 eV \qquad ( Numerical Formula 13 C )$$

In an exemplary arrangement of the organic EL device of the exemplary embodiment, the first host material, the second host material, and the first luminescent compound satisfy a relationship of a numerical formula (Numerical Formula 2B) below.

$${T}_{1} ( D 1 ) > {T}_{1} ( H 1 ) > {T}_{1} ( H 2 ) \qquad ( Numerical Formula 2 B )$$

In an exemplary arrangement of the organic EL device of the exemplary embodiment, a triplet energy of the first luminescent compound or the second luminescent compound T₁(DX), the triplet energy of the first host material T₁(H1), and the triplet energy of second host material T₁(H2) satisfy a relationship of a numerical formula (Numerical Formula 10) below.

$$2.6 eV > {T}_{1} ( D X ) > {T}_{1} ( H 1 ) > {T}_{1} ( H 2 ) \qquad ( Numerical Formula 10 )$$

In an exemplary arrangement of the organic EL device of the exemplary embodiment, the triplet energy of the first luminescent compound T₁(D1) satisfies a relationship of a numerical formula (Numerical Formula 10A) below.

$$2.6 eV > {T}_{1} ( D 1 ) > {T}_{1} ( H 1 ) > {T}_{1} ( H 2 ) \qquad ( Numerical Formula 10 A )$$

In an exemplary arrangement of the organic EL device of the exemplary embodiment, the triplet energy of the second luminescent compound T₁(D2) satisfies a relationship of a numerical formula (Numerical Formula 10B) below.

$$2.6 eV > {T}_{1} ( D 2 ) > {T}_{1} ( H 1 ) > {T}_{1} ( H 2 ) \qquad ( Numerical Formula 10 B )$$

In an exemplary arrangement of the organic EL device of the exemplary embodiment, the triplet energy of the first luminescent compound or the second luminescent compound T₁(DX) and the triplet energy of the first host material T₁(H1) satisfy a relationship of a numerical formula (Numerical Formula 11) below.

$$0 eV < {T}_{1} ( DX ) - {T}_{1} ( H 1 ) < 0.6 eV \qquad ( Numerical Formula 11 )$$

In an exemplary arrangement of the organic EL device of the exemplary embodiment, the triplet energy of the first luminescent compound T₁(D1) satisfies a relationship of a numerical formula (Numerical Formula 11A) below.

$$0 eV < {T}_{1} ( D 1 ) - {T}_{1} ( H 1 ) < 0.6 eV \qquad ( Numerical Formula 11 A )$$

In an exemplary arrangement of the organic EL device of the exemplary embodiment, the triplet energy of the second luminescent compound T₁(D2) satisfies a relationship of a numerical formula (Numerical Formula 11B) below.

$$0 eV < {T}_{1} ( D 2 ) - {T}_{1} ( H 2 ) < 0.8 eV \qquad ( Numerical Formula 11 B )$$

In an exemplary arrangement of the organic EL device of the exemplary embodiment, the second emitting layer contains 0.5 mass % or more of the second luminescent compound with respect to the total mass of the second emitting layer.

In an exemplary arrangement of the organic EL device of the exemplary embodiment, the second emitting layer contains the second luminescent compound at 10 mass % or less, 7 mass % or less, or 5 mass % or less with respect to the total mass of the second emitting layer.

In an exemplary arrangement of the organic EL device of the exemplary embodiment, the second emitting layer contains the second compound as the second host material at 60 mass % or more, 70 mass % or more, 80 mass % or more, 90 mass % or more, or 95 mass % or more with respect to the total mass of the second emitting layer.

In an exemplary arrangement of the organic EL device of the exemplary embodiment, the second emitting layer preferably contains 99 mass % or less of the second host material with respect to the total mass of the second emitting layer.

When the second emitting layer contains the second host material and the second luminescent compound, the upper limit of the total of the content ratios of the second host material and the second luminescent compound is 100 mass %.

The second emitting layer of the exemplary embodiment may further contain any other material than the second host material and the second luminescent compound.

In an exemplary arrangement of the organic EL device of the exemplary embodiment, the second emitting layer may contain a single type of the second host material or may contain two or more types of the second host material. In an exemplary arrangement of the organic EL device of the exemplary embodiment, the second emitting layer may contain a single type of the second luminescent compound or may contain two or more types of the second luminescent compound.

In an exemplary arrangement of the organic EL device of the exemplary embodiment, the second emitting layer may only contain the second host material and the second luminescent compound.

In an exemplary arrangement of the organic EL device of the exemplary embodiment, the second emitting layer has a film thickness of 5 nm or more or 10 nm or more. When the film thickness of the second emitting layer is 5 nm or more, it is easy to inhibit triplet excitons having transferred from the first emitting layer to the second emitting layer from returning to the first emitting layer. Further, when the film thickness of the second emitting layer is 5 nm or more, triplet excitons can be sufficiently separated from the recombination portion in the first emitting layer.

In the organic EL device according to the exemplary embodiment, the film thickness of the second emitting layer is preferably 25 nm or less. When the film thickness of the second emitting layer is 25 nm or less, a density of the triplet excitons in the second emitting layer is improved to cause the TTF phenomenon more easily.

In an exemplary arrangement of the organic EL device of the exemplary embodiment, the second emitting layer has a film thickness in a range from 5 to 25 nm.

### Second Host Material

In the organic EL device according to the exemplary embodiment, the second host material, which is not particularly limited, is exemplified by the second compound represented by a formula (2) below.

In an exemplary arrangement of the organic EL device of the exemplary embodiment, the second host material is a compound represented by the formula (2) below.

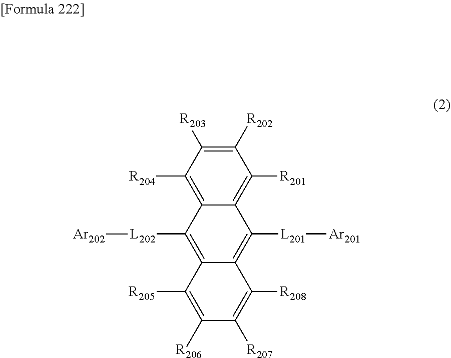

In the formula (2):
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₆), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms; and
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms.

In the second host material, R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₆ are present, the plurality of R₉₀₆ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In an exemplary arrangement of the organic EL device of the exemplary embodiment, the second host material is a compound having, in a molecule, at least one group represented by a formula (HY1) below.

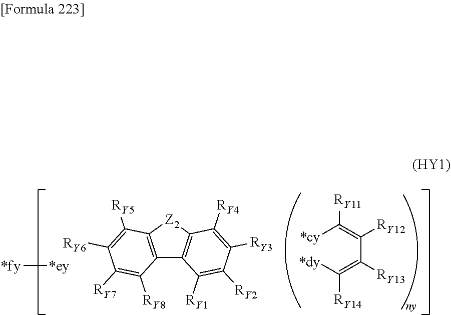

In the formula (HY1):
R_{Y1} to R_{Y8} and R_{Y11} to R_{Y14} are each independently a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms;
ny is 0 or 1;
when ny is 0, one selected from R_{Y1} to R_{Y8} is a single bond with *ey;
when ny is 1, one of R_{Y1} and R_{Y2}, one of R_{Y2} and R_{Y3}, or one of R_{Y3} and R_{Y4} is a single bond with *cy; the other of R_{Y1} and R_{Y2}, the other of R_{Y2} and R_{Y3}, or the other of R_{Y3} and R_{Y4} is a single bond with *dy; and one selected from R_{Y5} to R_{Y8}, R_{Y11} to R_{Y14}, and R_{Y1} to R_{Y4} being neither the single bond with *cy nor the single bond with
* dy is a single bond with *ey;
Z₂ is an oxygen atom or a sulfur atom; and
*fy represents a bonding position to an atom in the second host material.

In an exemplary arrangement of the organic EL device of the exemplary embodiment, when ny in the formula (HY1) is zero, the group represented by the formula (HY1) is represented by a formula (HY10) below.

In an exemplary arrangement of the organic EL device of the exemplary embodiment, the second host material is a compound having, in a molecule, at least one group represented by the formula (HY10) below.

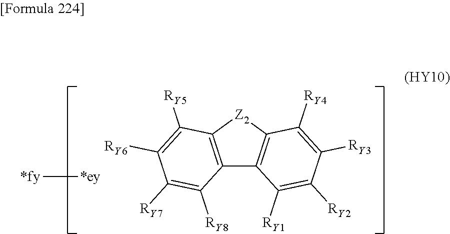

In the formula (HY10):
R_{Y1} to R_{Y8}, and Z₂ respectively represent the same as R_{Y1} to R_{Y8}, and Z₂ in the formula (HY1);
one selected from R_{Y1} to R_{Y8} is a single bond with *ey; and
*fy represents a bonding position to an atom in the second host material.

In an exemplary arrangement of the organic EL device of the exemplary embodiment, the second host material is a compound having, in a molecule, at least one group represented by the formula (HY1), in which ny is 1.

In an exemplary arrangement of the organic EL device of the exemplary embodiment, the second host material is a compound having, in a molecule, at least one group selected from the group consisting of groups represented by formulae (HY11), (HY12), and (HY13) below.

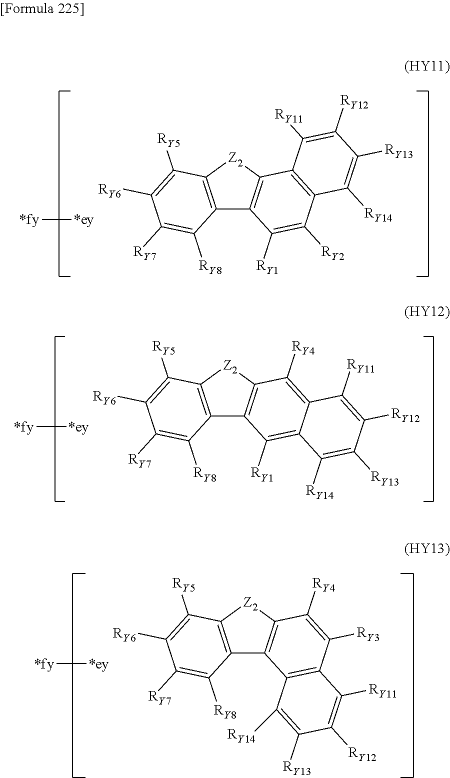

In the formulae (HY11), (HY12), and (HY13):
R_{Y1} to R_{Y8}, R_{Y11} to R_{Y14}, and Z₂ respectively represent the same as R_{Y1} to R_{Y8}, R_{Y11} to R_{Y14}, and Z₂ in the formula (HY1);
one of R_{Y1} to R_{Y8} and R_{Y11} to R_{Y14} is a single bond with *ey; and
*fy represents a bonding position to an atom in the second host material.

In an exemplary arrangement of the organic EL device of the exemplary embodiment, the second host material is a compound represented by the formula (2), the compound represented by the formula (2) having, in a molecule, at least one group represented by the formula (HY1).

In an exemplary arrangement of the organic EL device of the exemplary embodiment, at least one of Ar₂₀₁ or Ar₂₀₂ in the formula (2) is a group represented by the formula (HY1).

In an exemplary arrangement of the organic EL device of the exemplary embodiment, Ar₂₀₁ or Ar₂₀₂ in the formula (2) is a group represented by the formula (HY1).

In an exemplary arrangement of the organic EL device of the exemplary embodiment, the second host material is a compound represented by the formula (2), the compound represented by the formula (2) having, in a molecule, at least one group selected from the group consisting of groups represented by the formulae (HY11), (HY12), and (HY13).

In an exemplary arrangement of the organic EL device of the exemplary embodiment, at least one of Ar₂₀₁ or Ar₂₀₂ in the formula (2) is a group selected from the group consisting of groups represented by the formulae (HY11), (HY12), and (HY13).

In an exemplary arrangement of the organic EL device of the exemplary embodiment, Ar₂₀₁ or Ar₂₀₂ in the formula (2) is a group selected from the group consisting of groups represented by the formulae (HY11), (HY12), and (HY13).

In an exemplary arrangement of the organic EL device of the exemplary embodiment, the second host material that has, in a molecule, at least one group selected from the group consisting of groups represented by the formulae (HY1), (HY10), (HY11), (HY12), and (HY13), has improved excitation resistance. Using such a second host material for the second emitting layer makes it easy to extend a lifetime of the organic EL device.

In an exemplary arrangement of the organic EL device of the exemplary embodiment, at least one hydrogen atom contained in the second host material is a deuterium atom.

In an exemplary arrangement of the organic EL device of the exemplary embodiment, at least one of R₂₀₁ to R₂₀₈ in the second host material is a deuterium atom.

In an exemplary arrangement of the organic EL device of the exemplary embodiment, at least one of hydrogen atoms in Ar₂₀₁, Ar₂₀₂, L₂₀₁, and L₂₀₂ in the second host material is a deuterium atom.

In an exemplary arrangement of the organic EL device of the exemplary embodiment, at least one of R_{Y1} to R_{Y8} or R_{Y11} to R_{Y14} in the second host material is a deuterium atom.

In an exemplary arrangement of the organic EL device of the exemplary embodiment, at least one hydrogen atom contained in the third compound is a deuterium atom, and at least one hydrogen atom contained in the second host material is a deuterium atom.

In an exemplary arrangement of the organic EL device of the exemplary embodiment, the groups specified to be “substituted or unsubstituted” in the second host material are each an unsubstituted group.

### Method for Producing Second Host Material

The second host material according to the exemplary embodiment can be produced by a known method or through a known alternative reaction using a known material(s) tailored for the target compound in accordance with the known method.

### Specific Examples of Second Host Material

Specific examples of the second host material according to the exemplary embodiment include compounds below. The invention, however, is not limited to the specific examples.

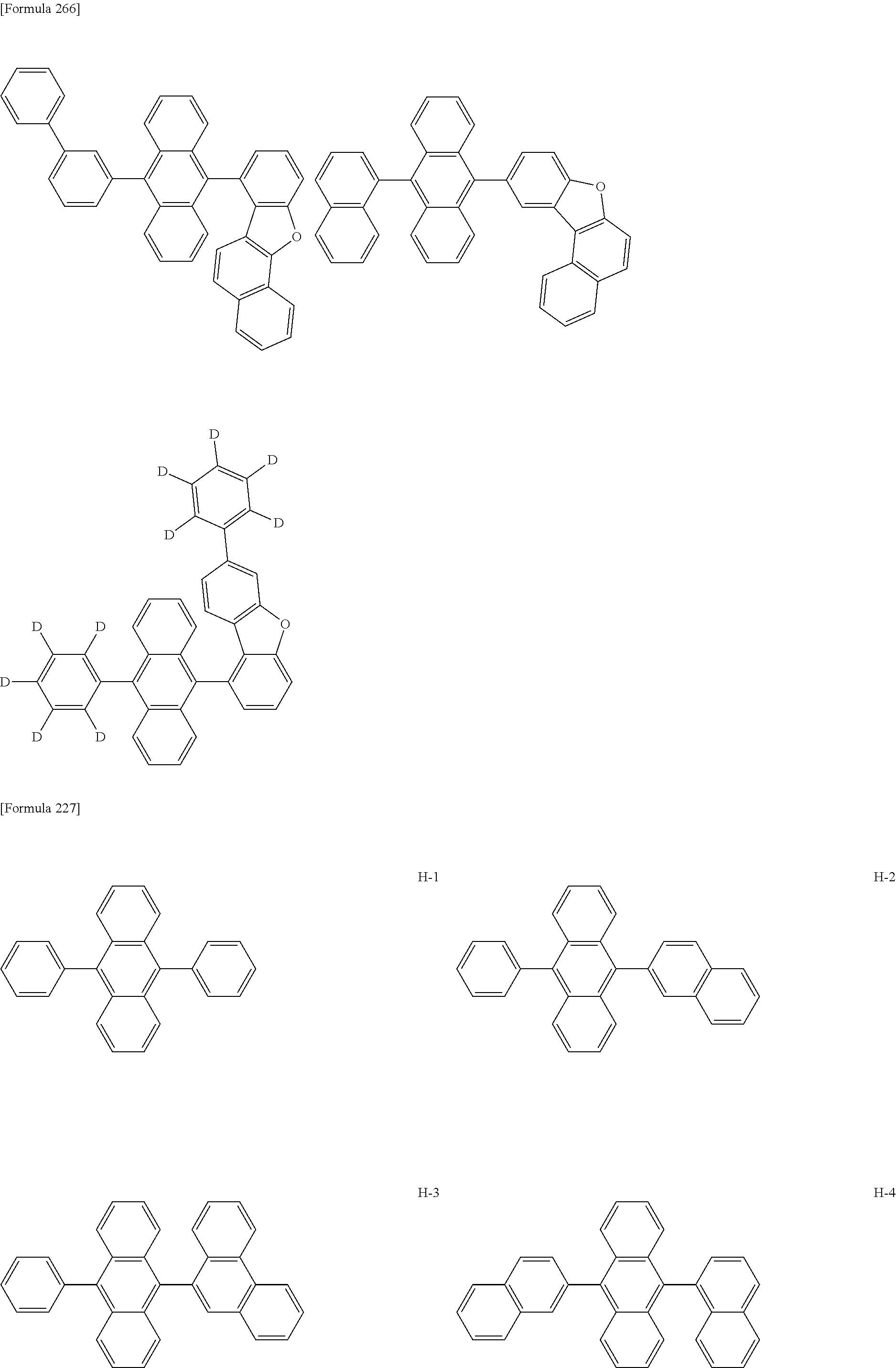

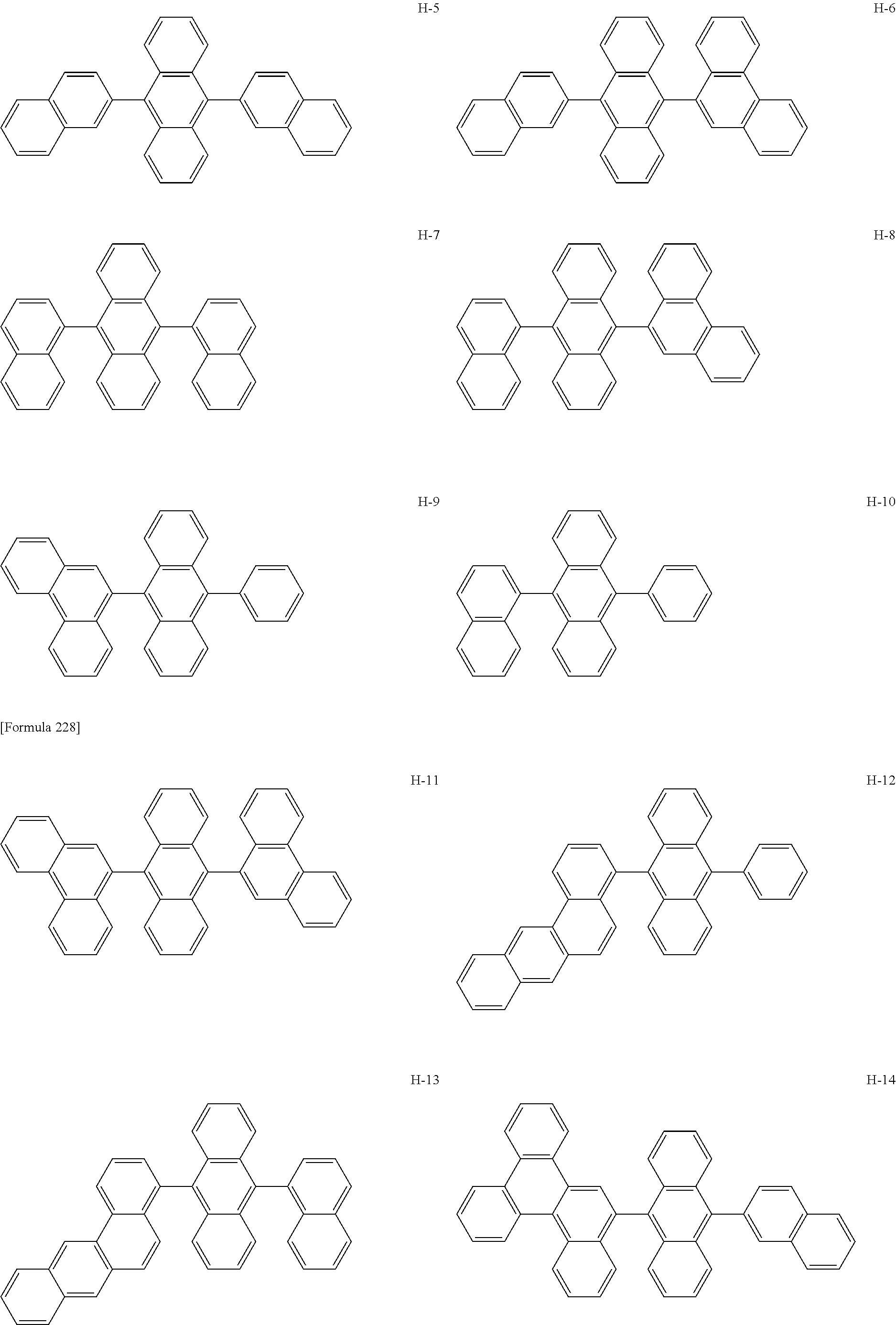

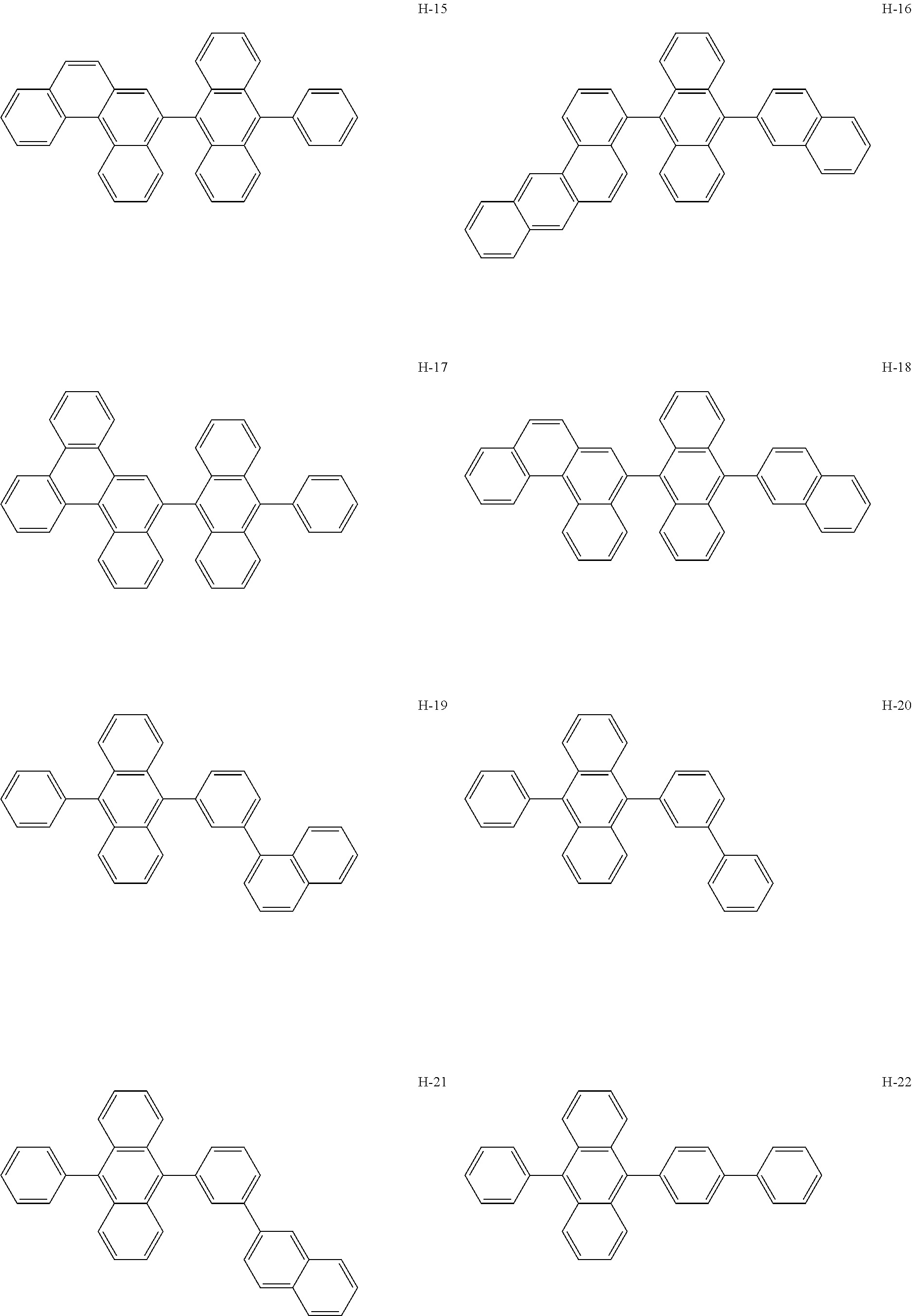

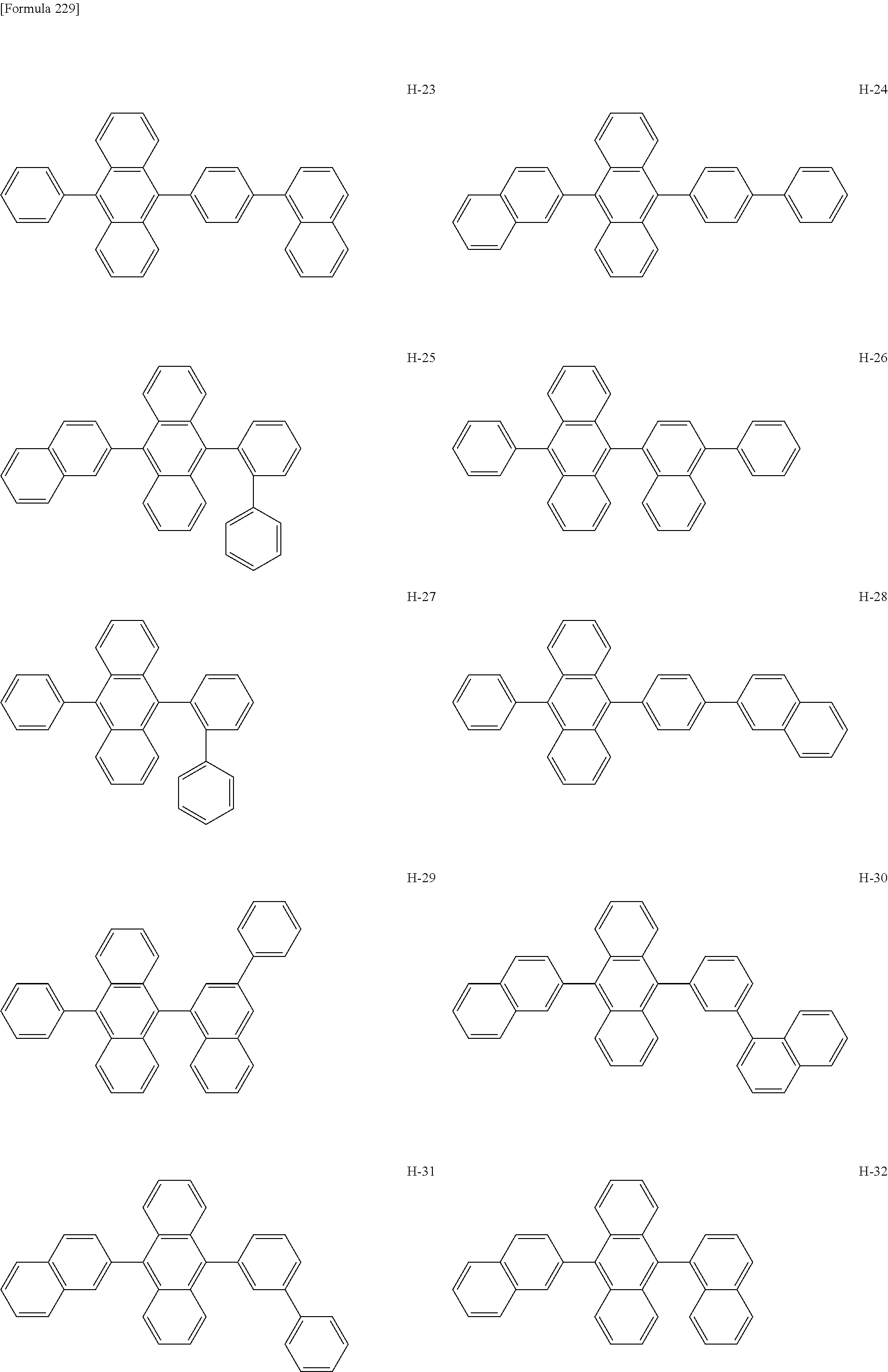

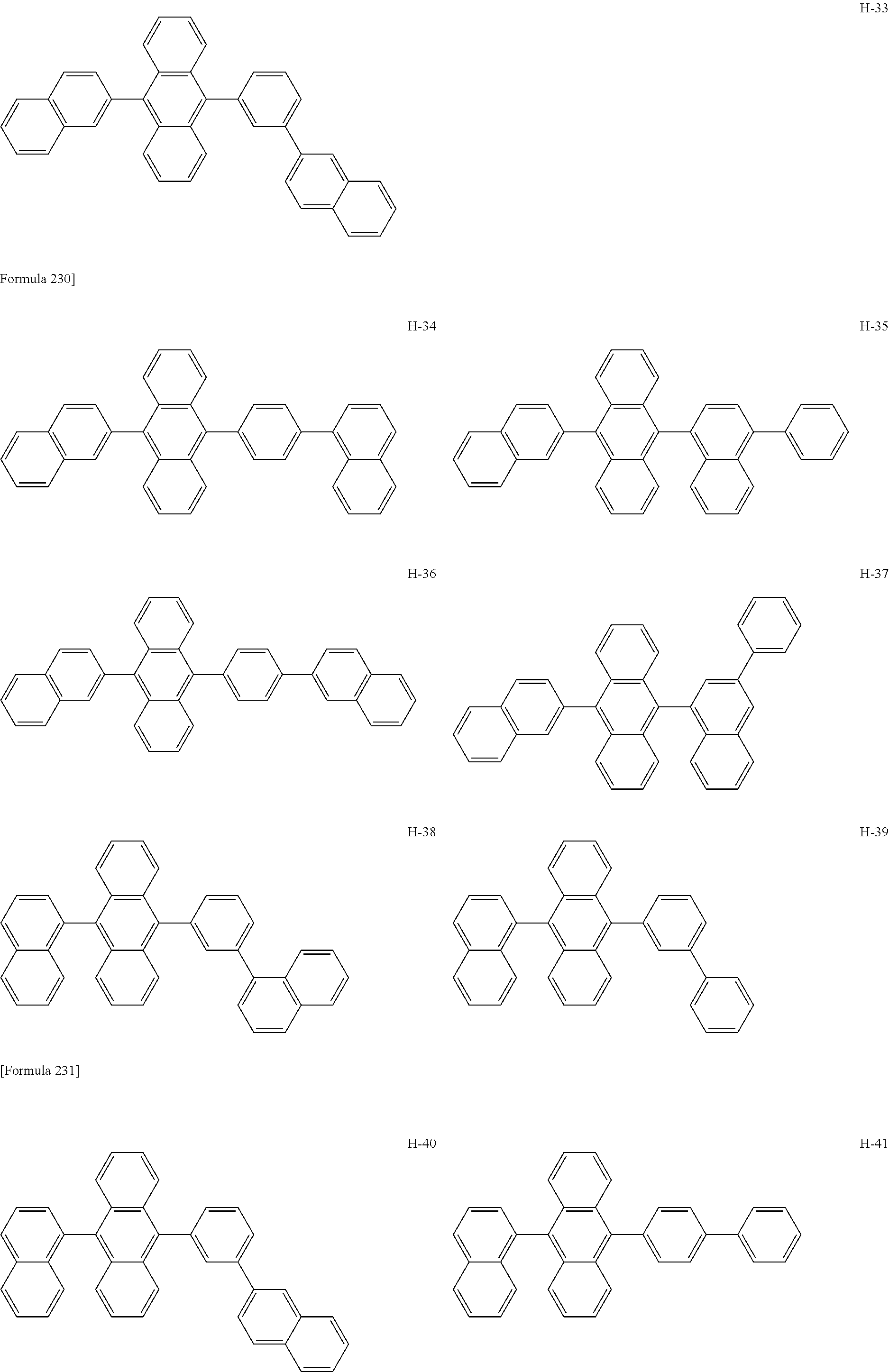

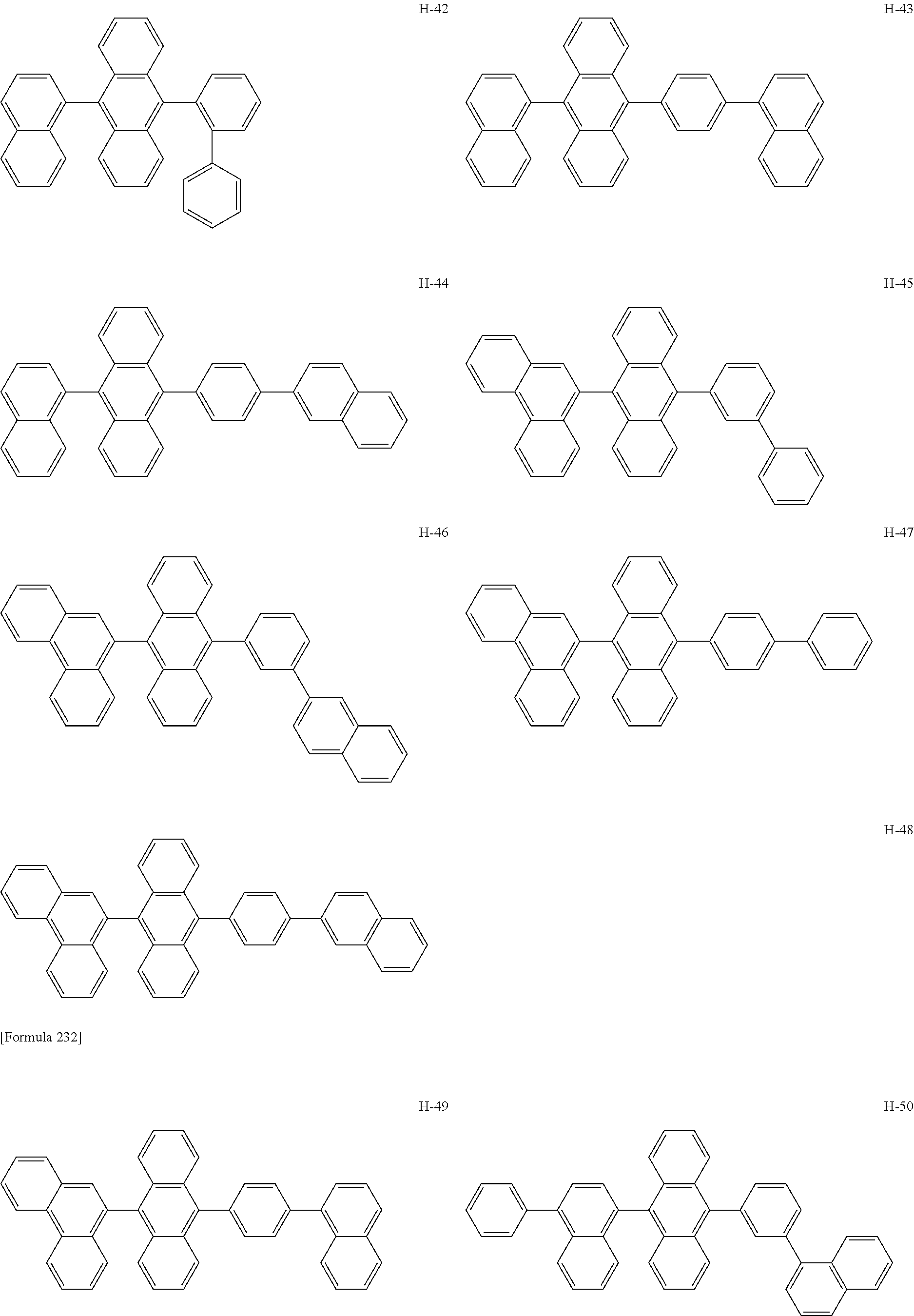

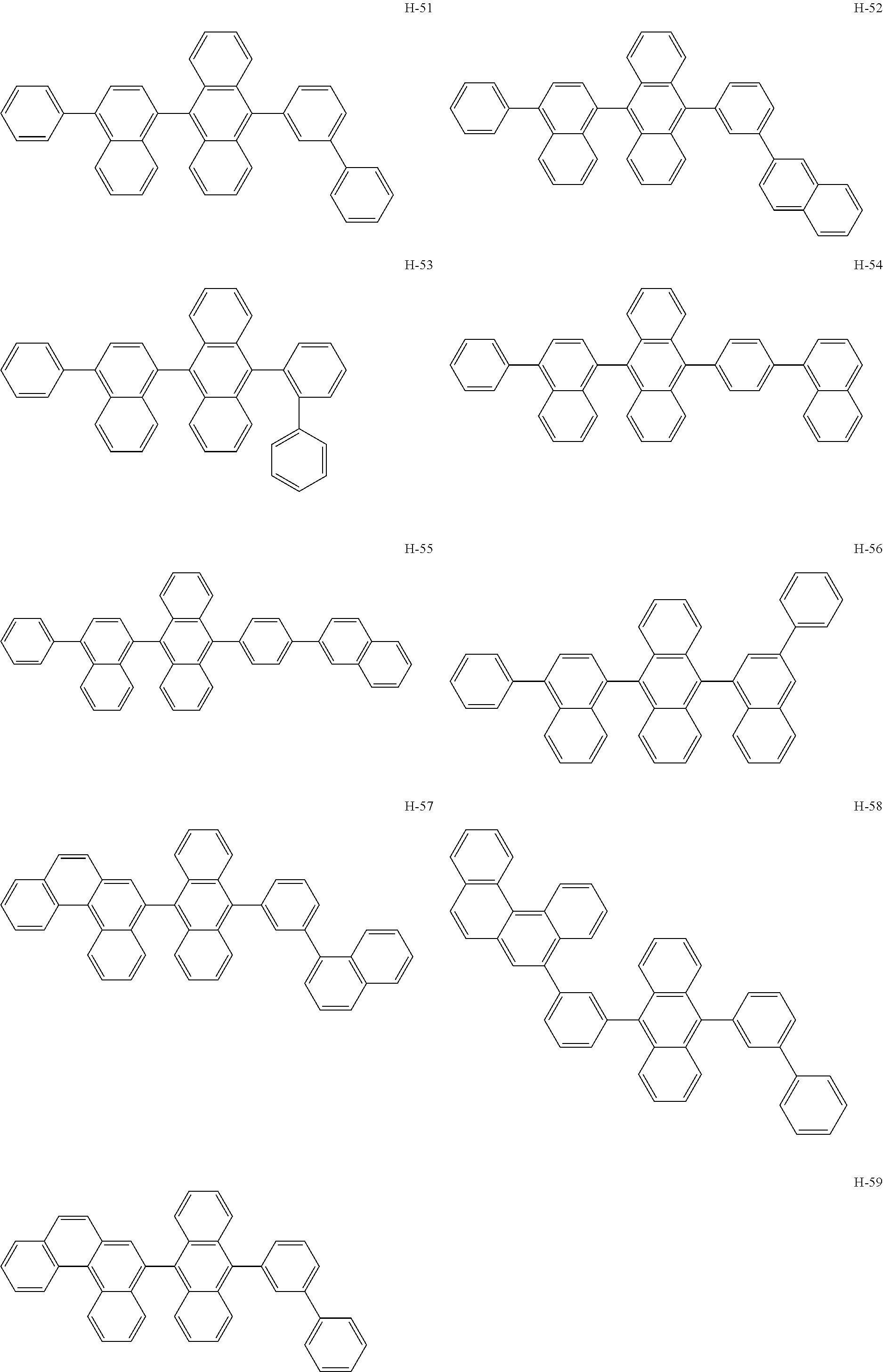

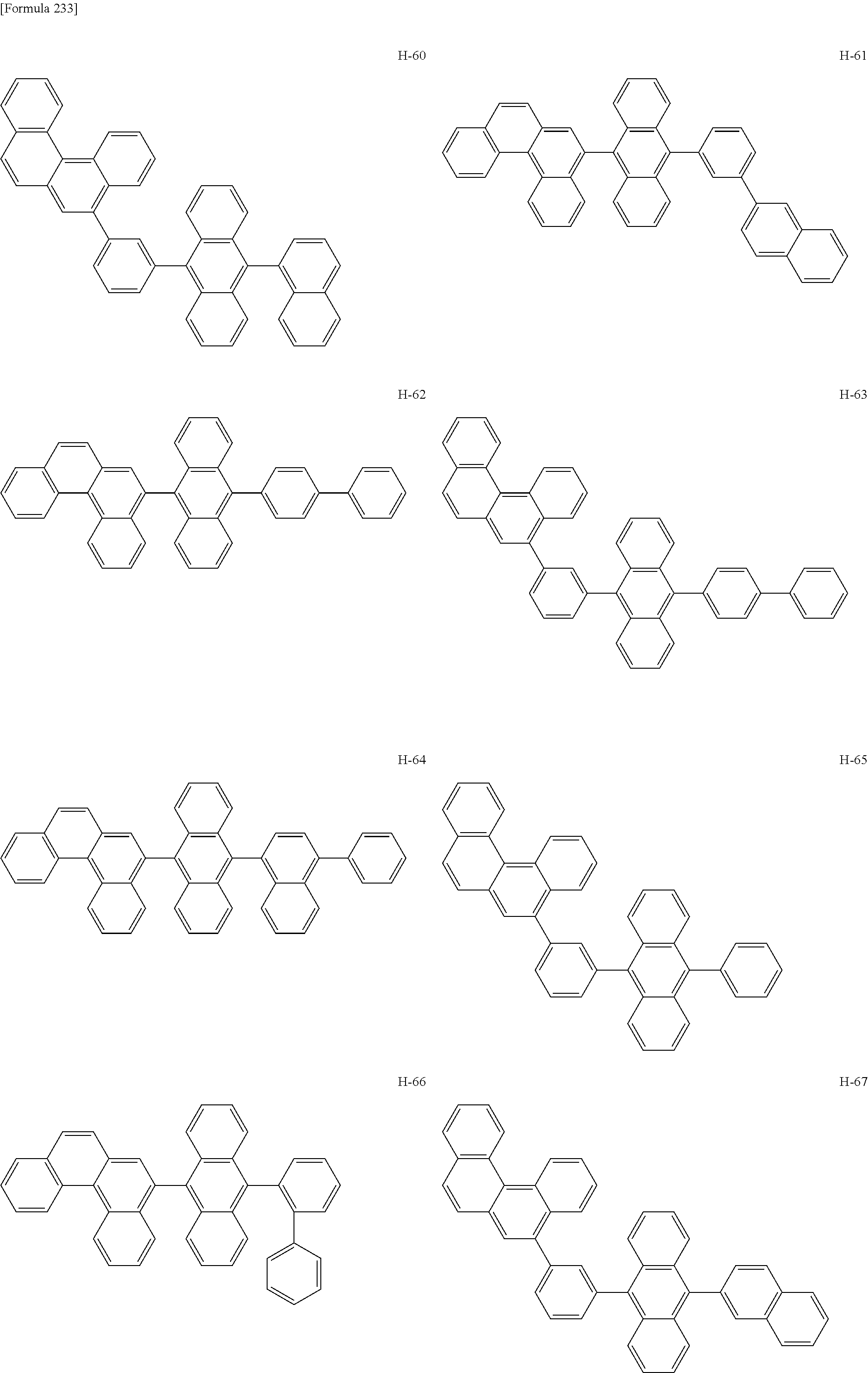

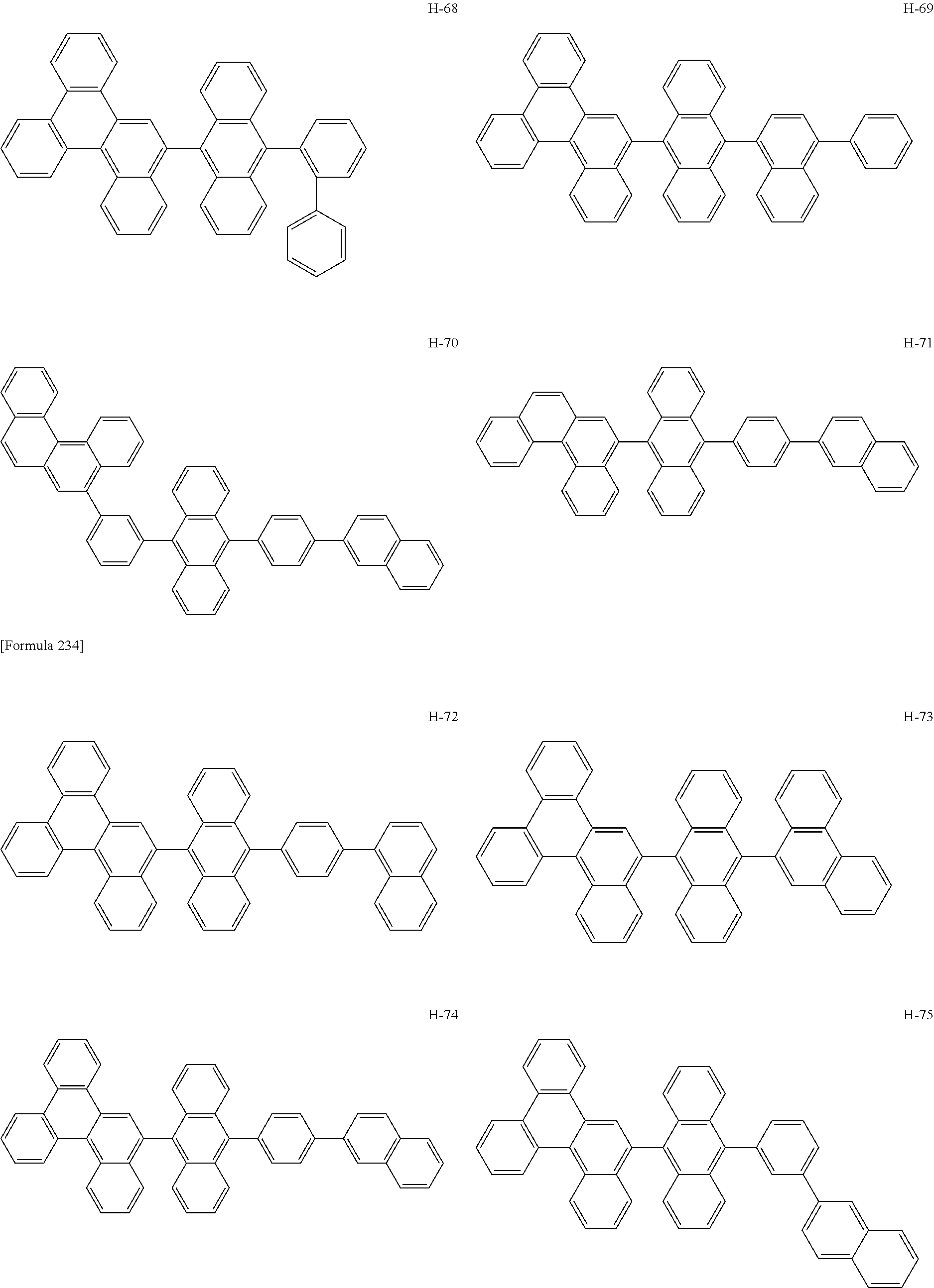

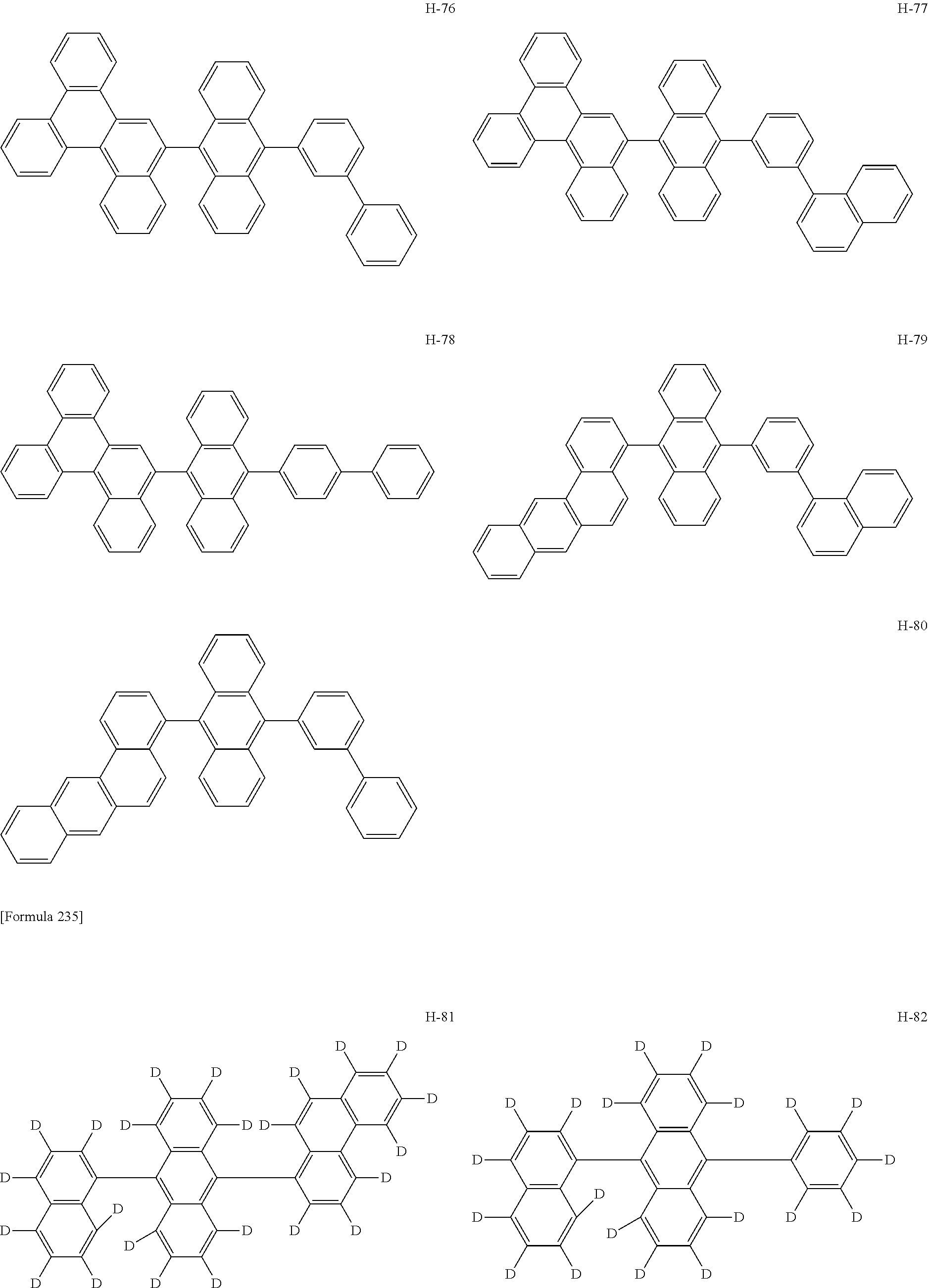

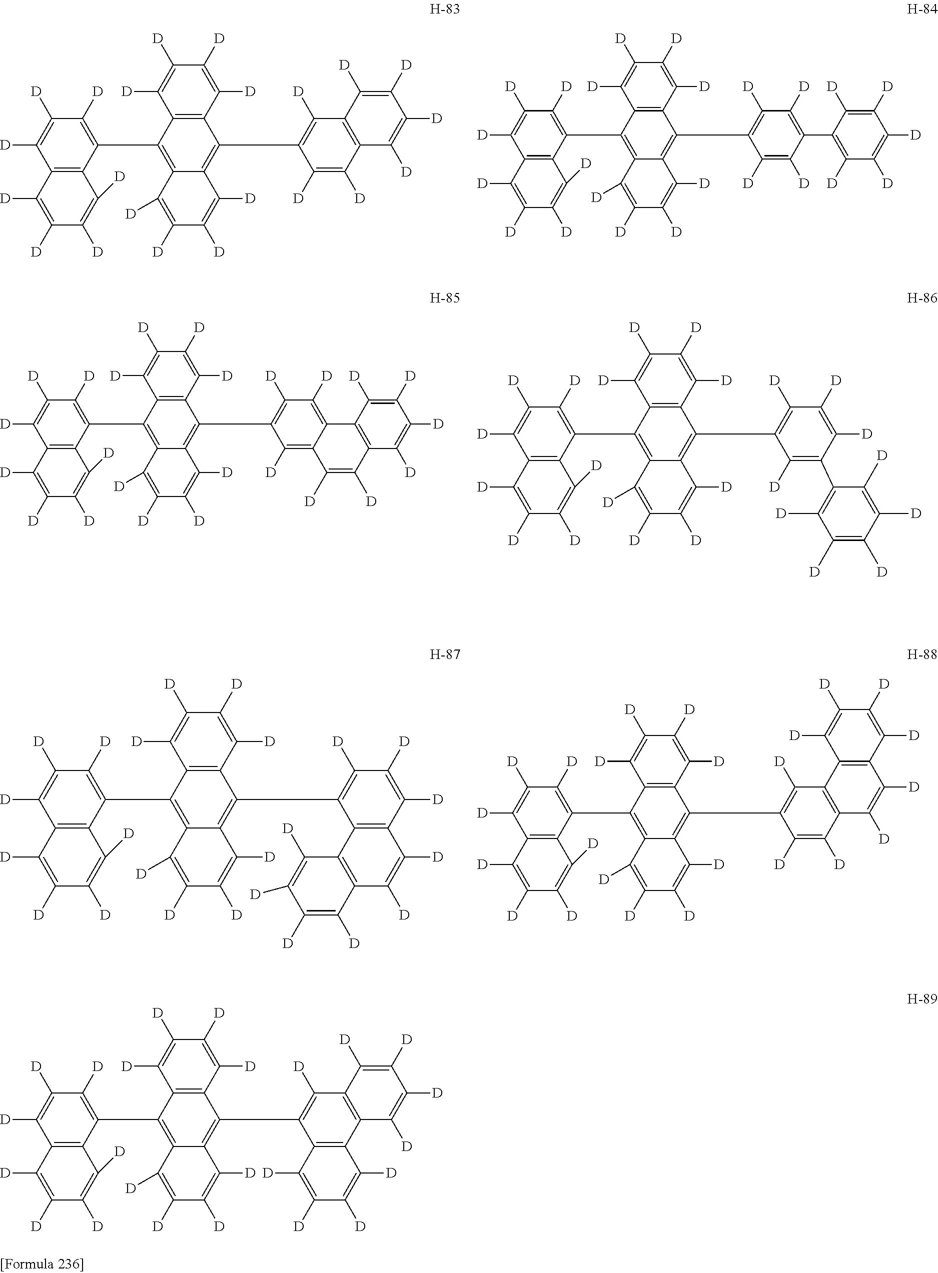

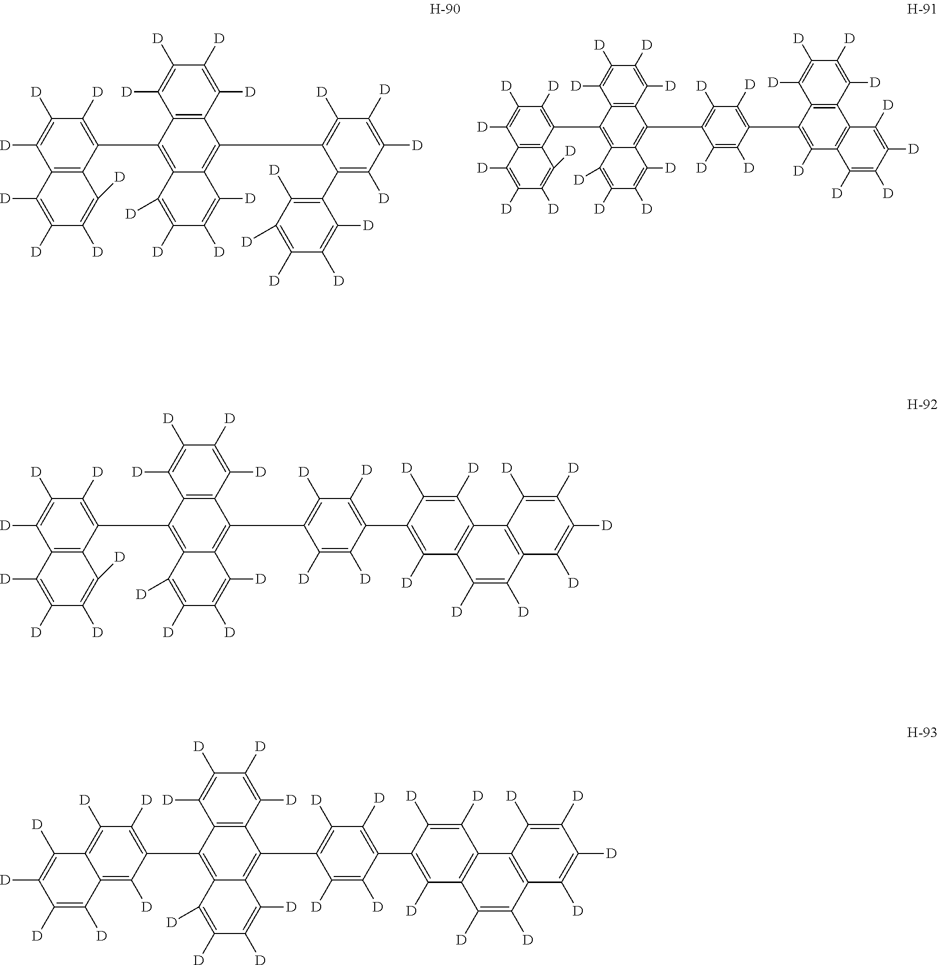

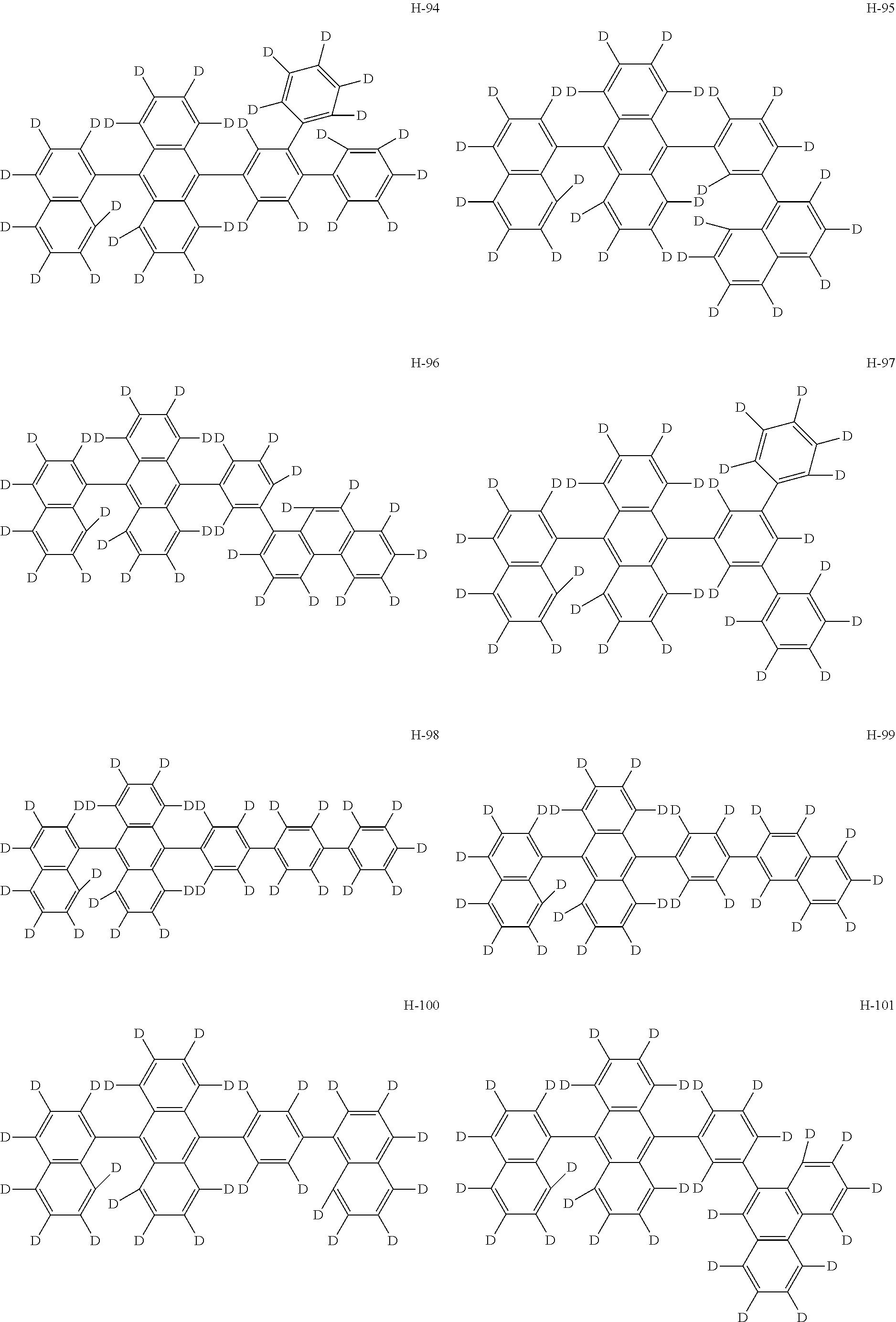

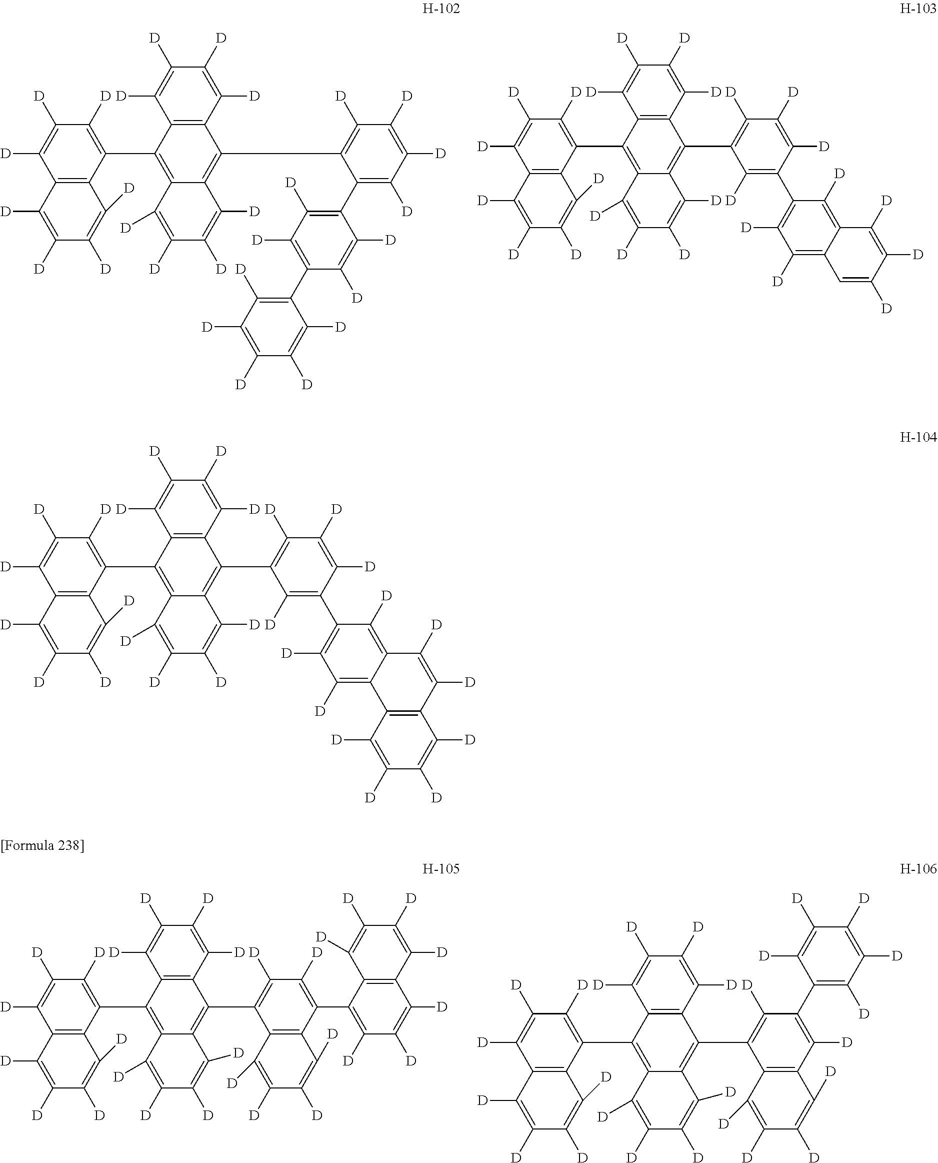

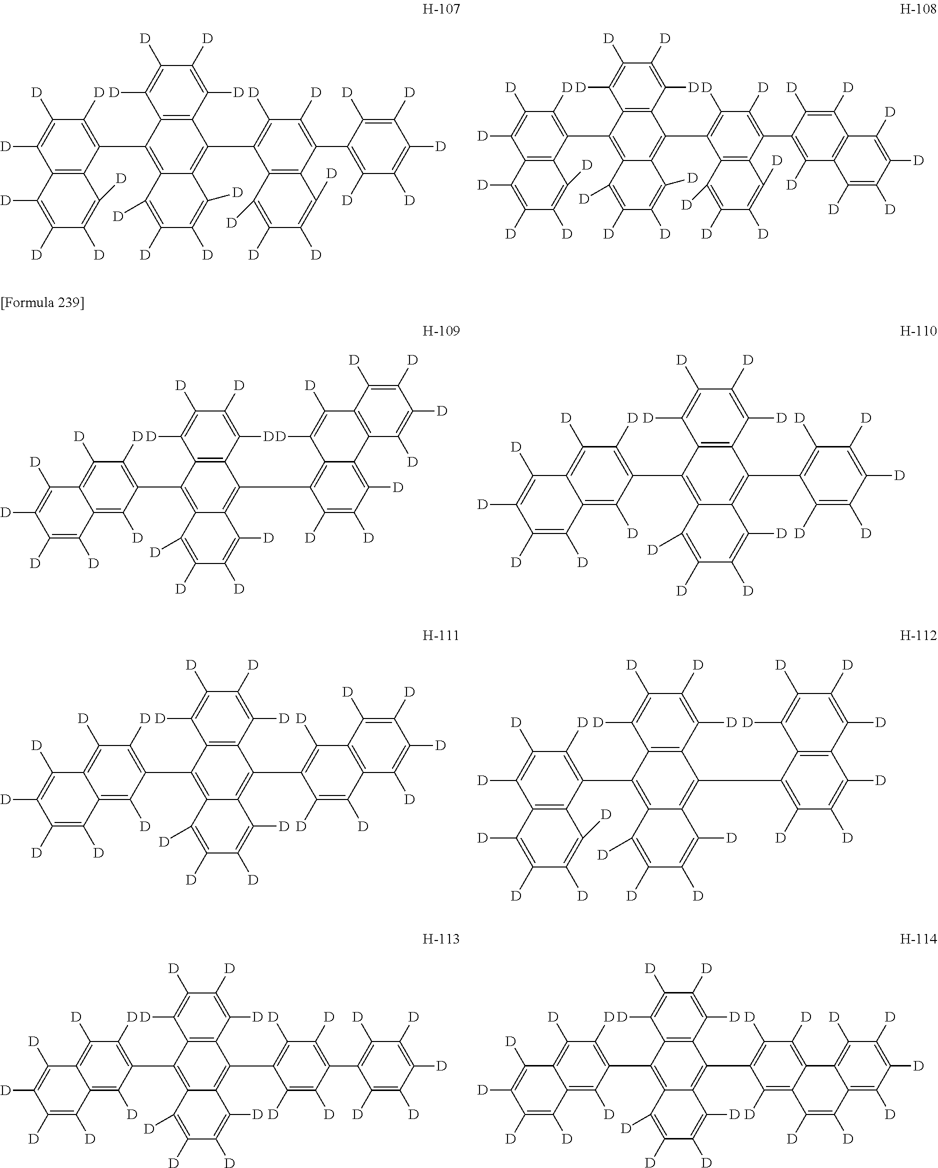

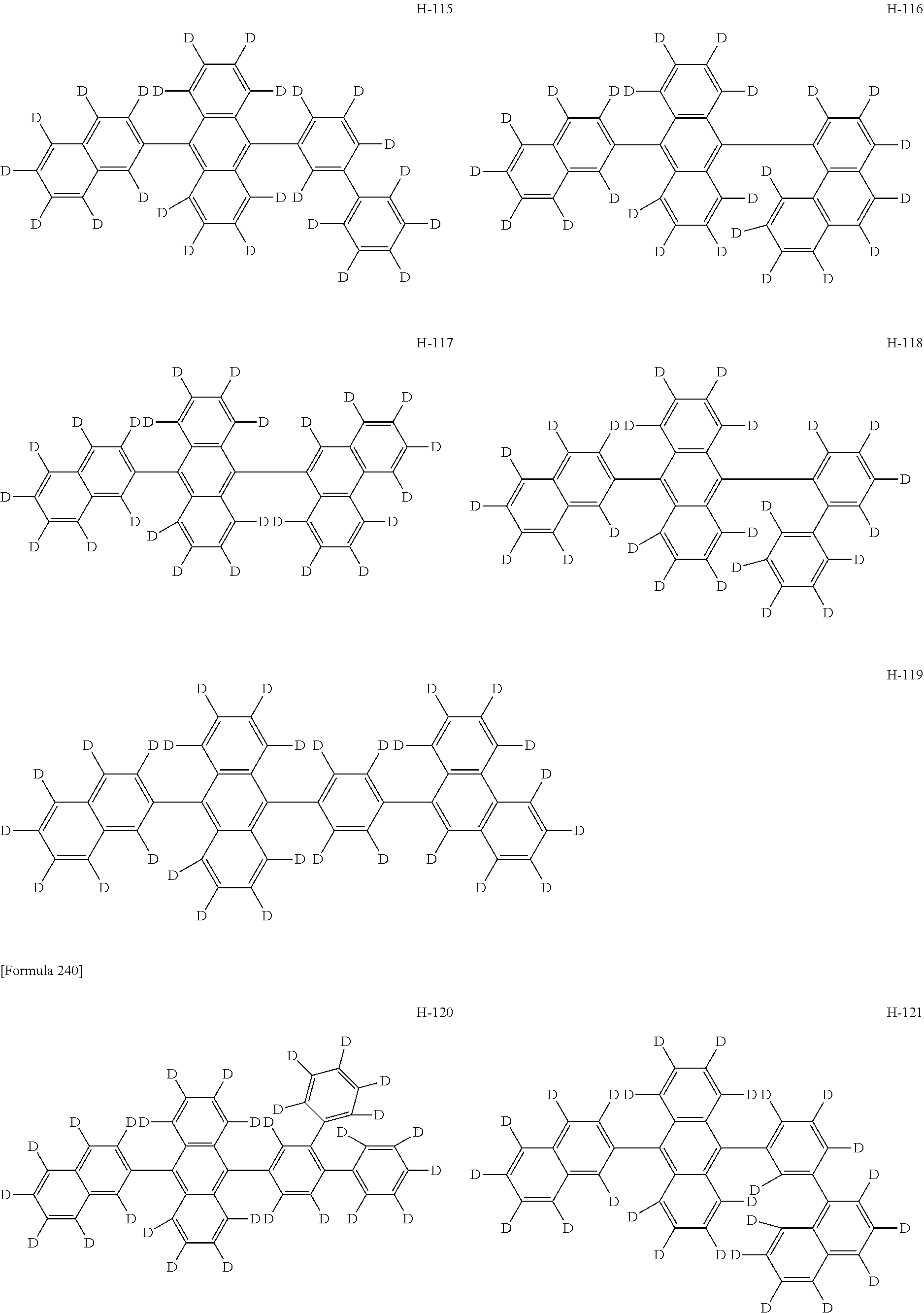

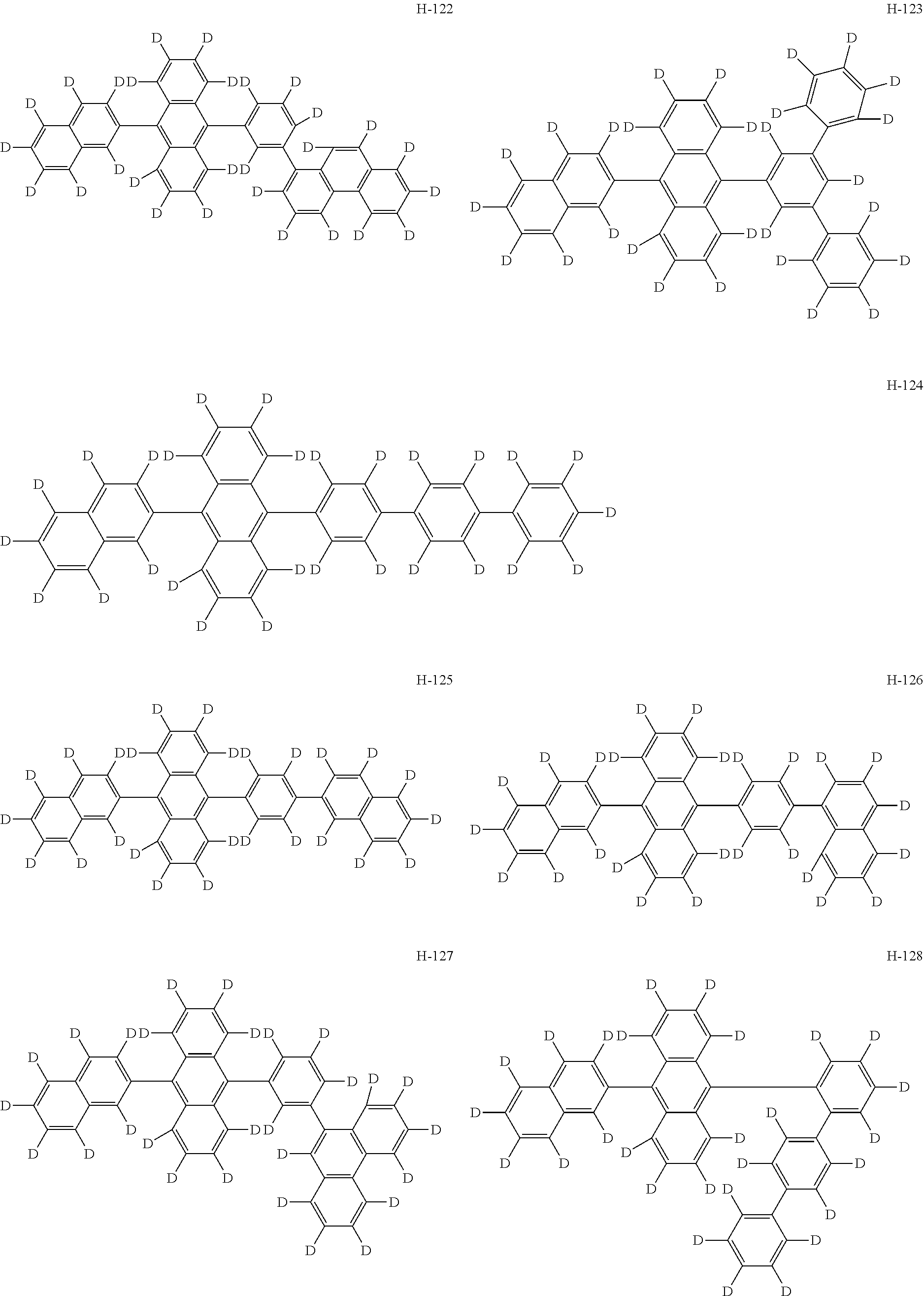

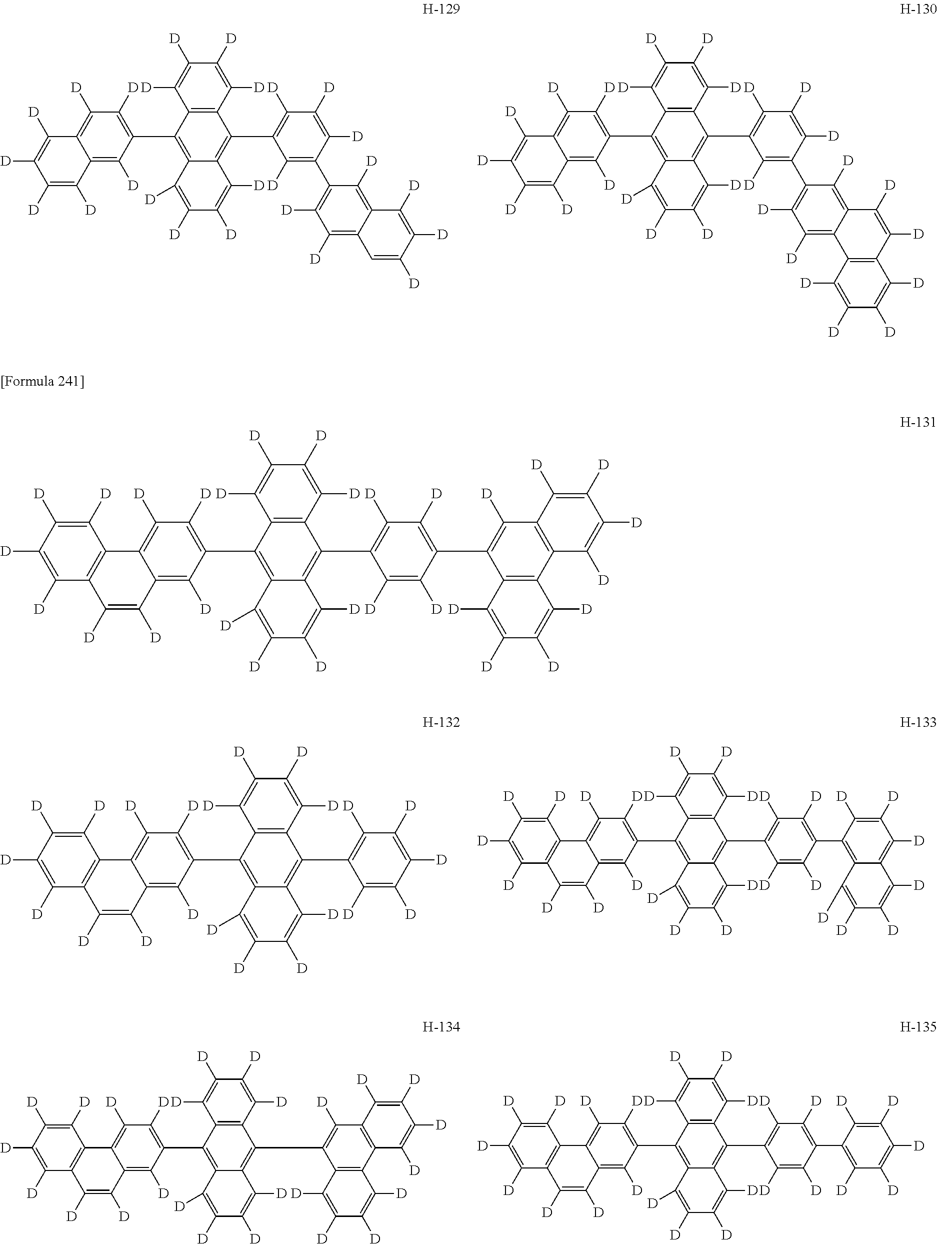

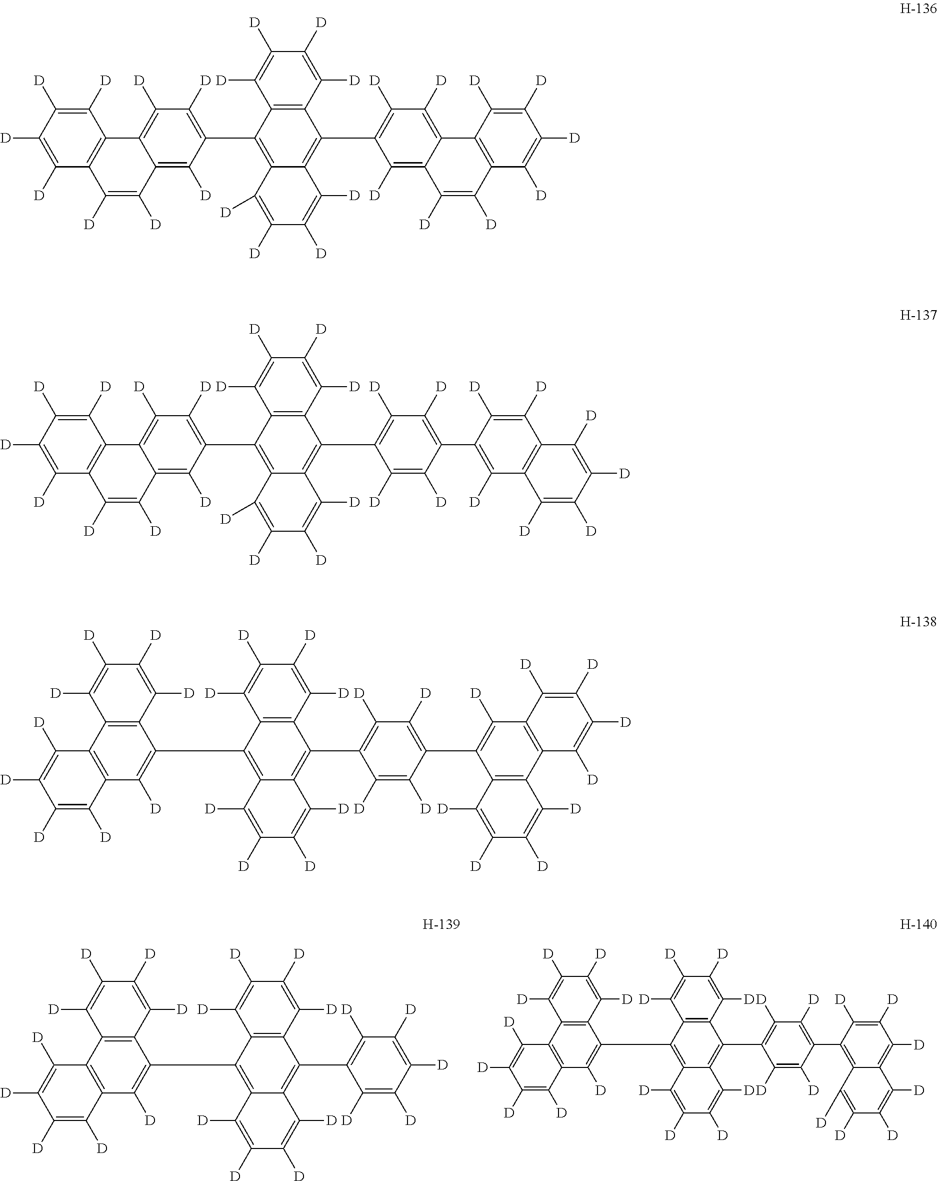

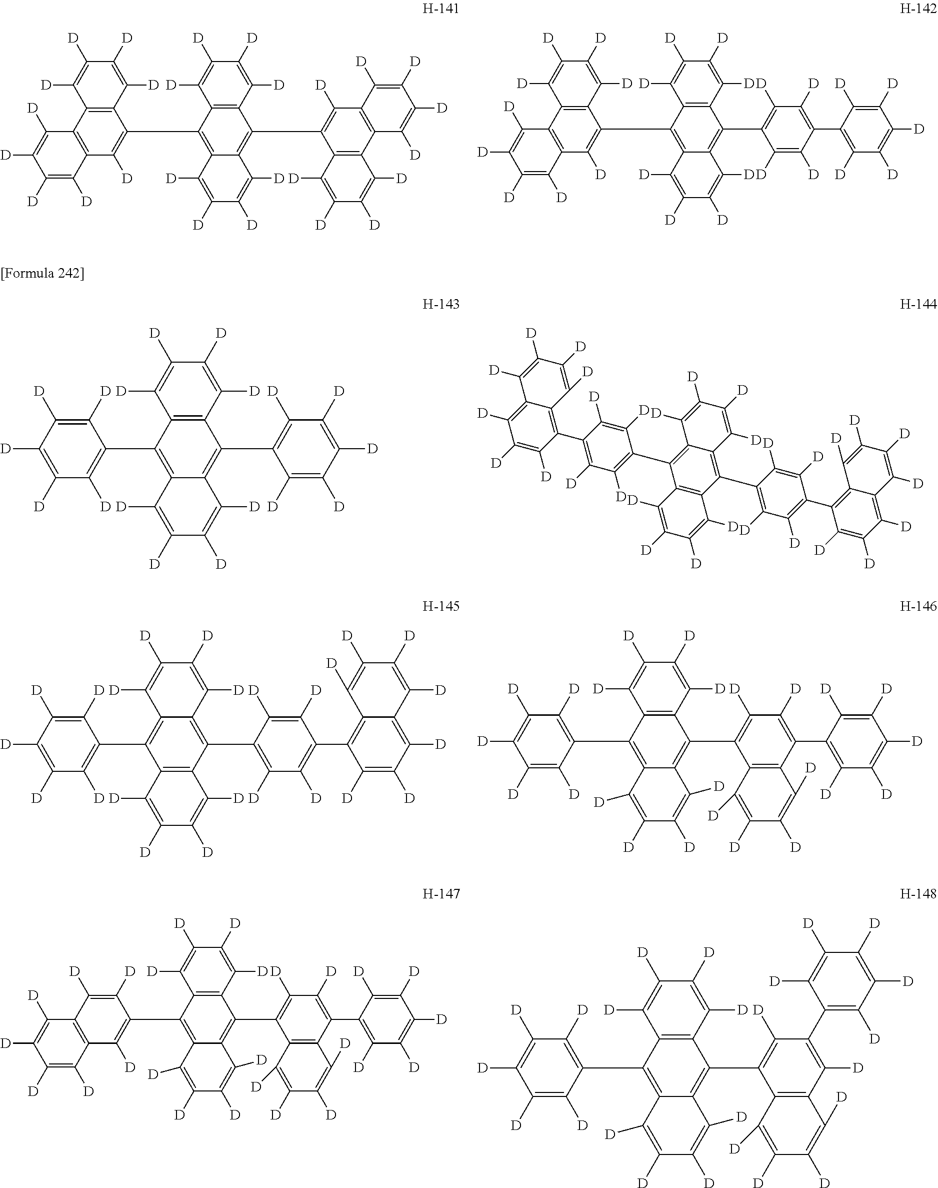

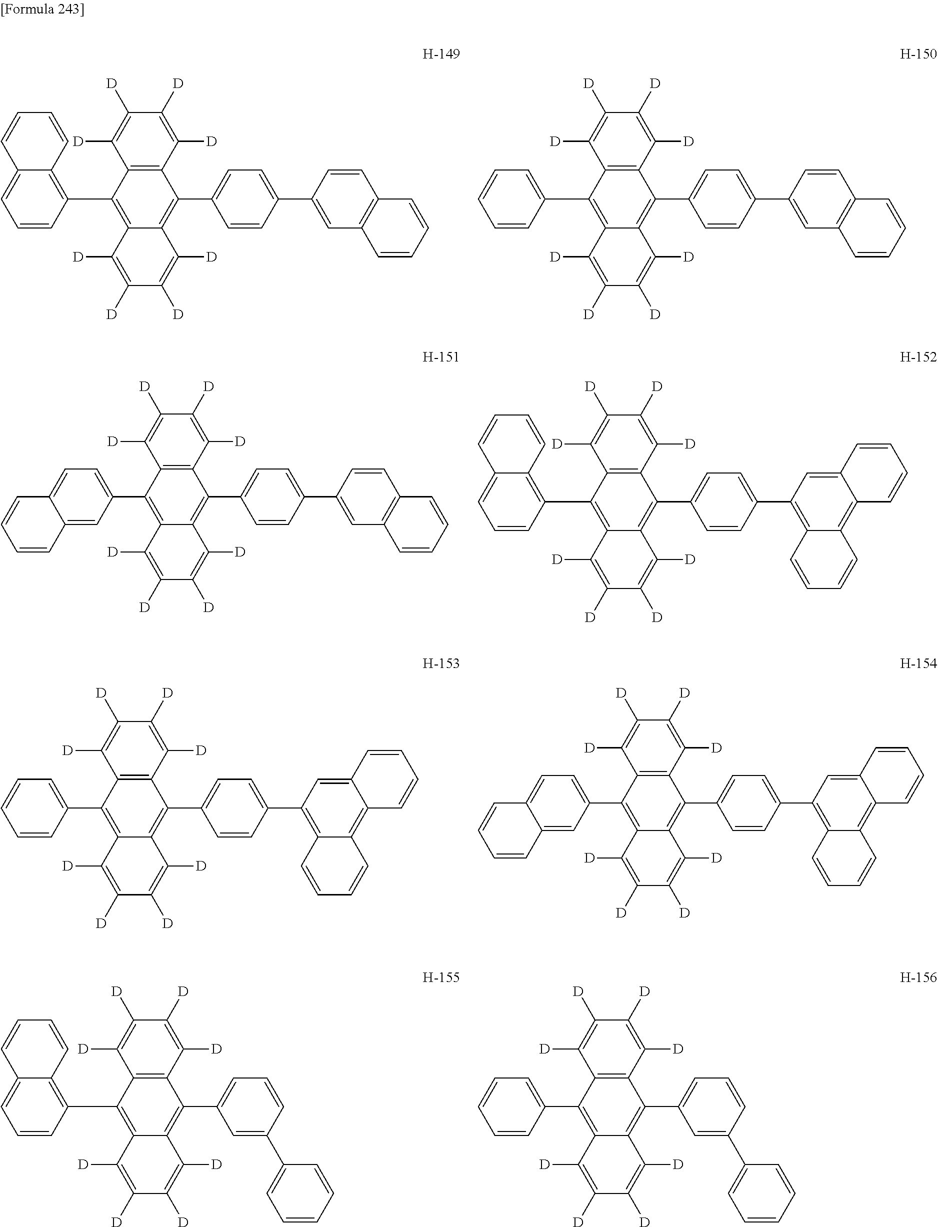

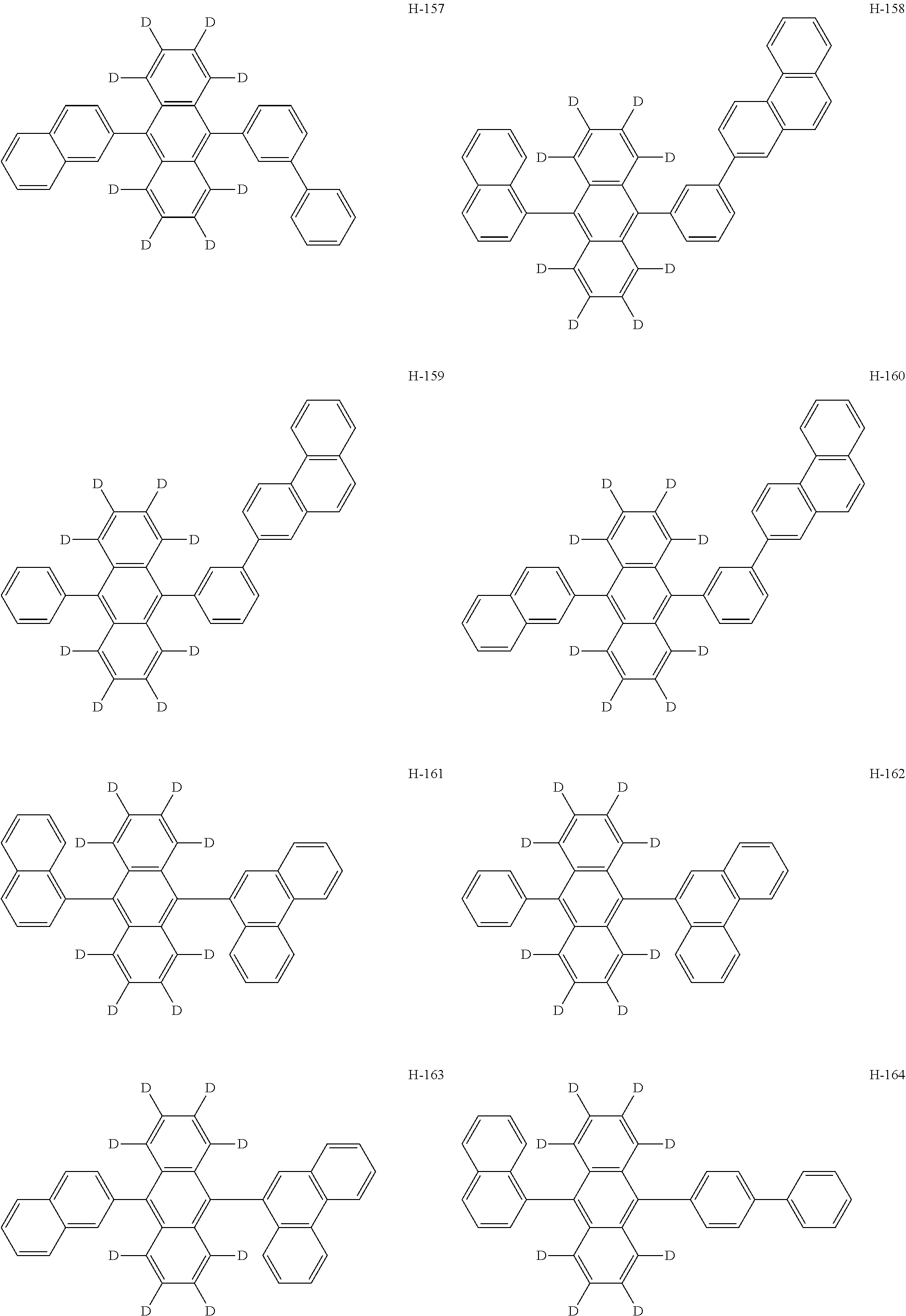

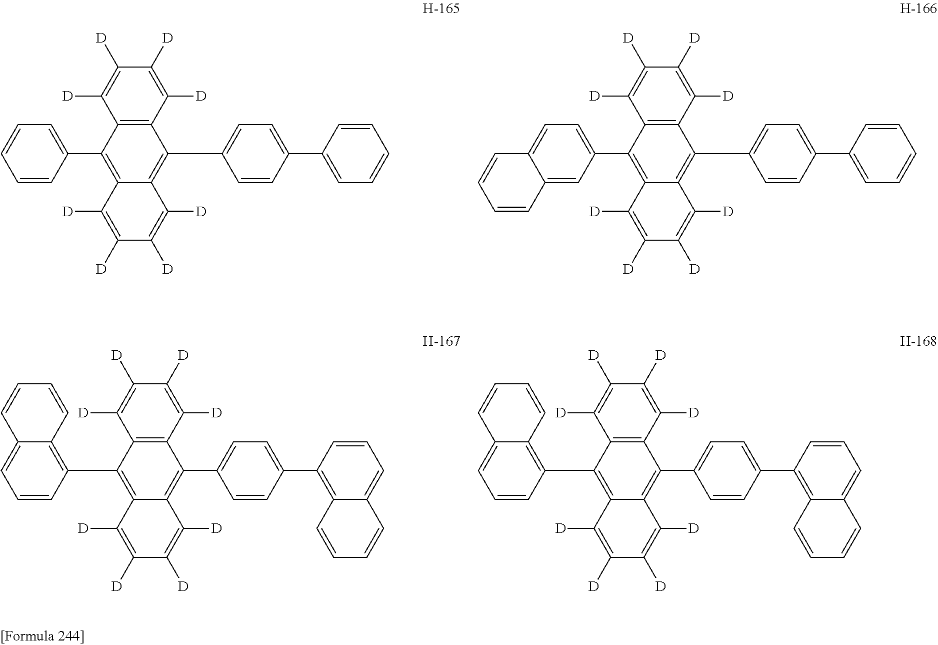 [SMILES: [2H]c1c([2H])c([2H])c2c(-c3ccc(-c4ccccc4)cc3)c3c([2H])c([2H])c([2H])c([2H])c3c(-c3ccccc3)c2c1[2H] ; [2H]c1c([2H])c([2H])c2c(-c3ccc4ccccc4c3)c3c([2H])c([2H])c([2H])c([2H])c3c(-c3ccc(-c4ccccc4)cc3)c2c1[2H] ; [2H]c1c([2H])c([2H])c2c(-c3cccc4ccccc34)c3c([2H])c([2H])c([2H])c([2H])c3c(-c3ccc(-c4cccc5ccccc45)cc3)c2c1[2H] ; [2H]c1c([2H])c([2H])c2c(-c3cccc4ccccc34)c3c([2H])c([2H])c([2H])c([2H])c3c(-c3ccc(-c4cccc5ccccc45)cc3)c2c1[2H]]

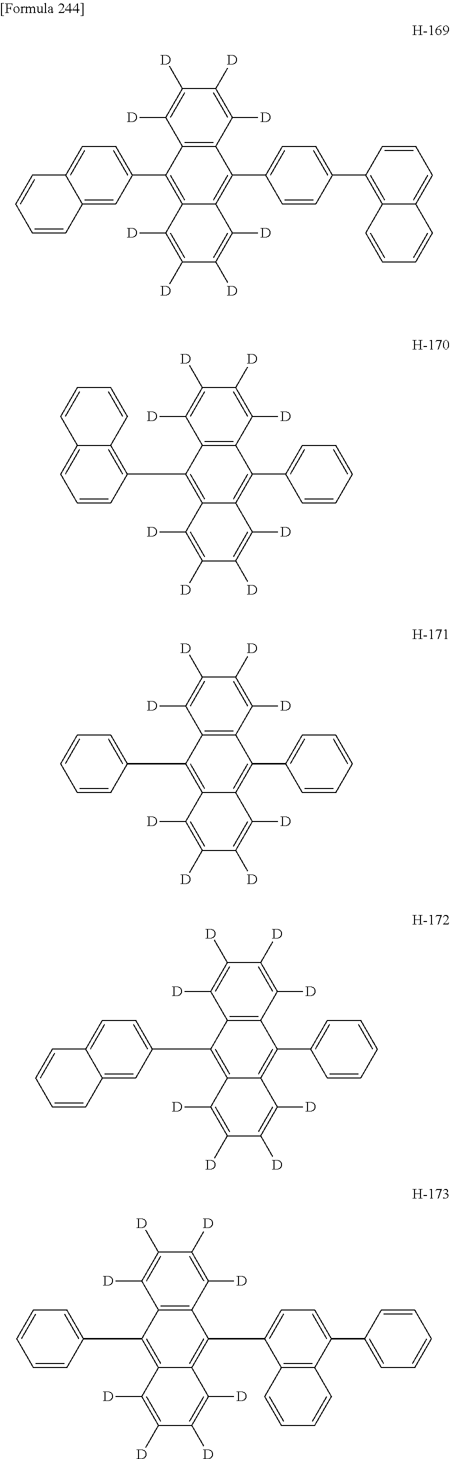 [SMILES: [2H]c1c([2H])c([2H])c2c(-c3ccc4ccccc4c3)c3c([2H])c([2H])c([2H])c([2H])c3c(-c3ccc(-c4cccc5ccccc45)cc3)c2c1[2H] ; [2H]c1c([2H])c([2H])c2c(-c3cccc4ccccc34)c3c([2H])c([2H])c([2H])c([2H])c3c(-c3ccccc3)c2c1[2H] ; [2H]c1c([2H])c([2H])c2c(-c3ccccc3)c3c([2H])c([2H])c([2H])c([2H])c3c(-c3ccccc3)c2c1[2H] ; [2H]c1c([2H])c([2H])c2c(-c3ccc4ccccc4c3)c3c([2H])c([2H])c([2H])c([2H])c3c(-c3ccccc3)c2c1[2H] ; [2H]c1c([2H])c([2H])c2c(-c3ccc(-c4ccccc4)c4ccccc34)c3c([2H])c([2H])c([2H])c([2H])c3c(-c3ccccc3)c2c1[2H]]

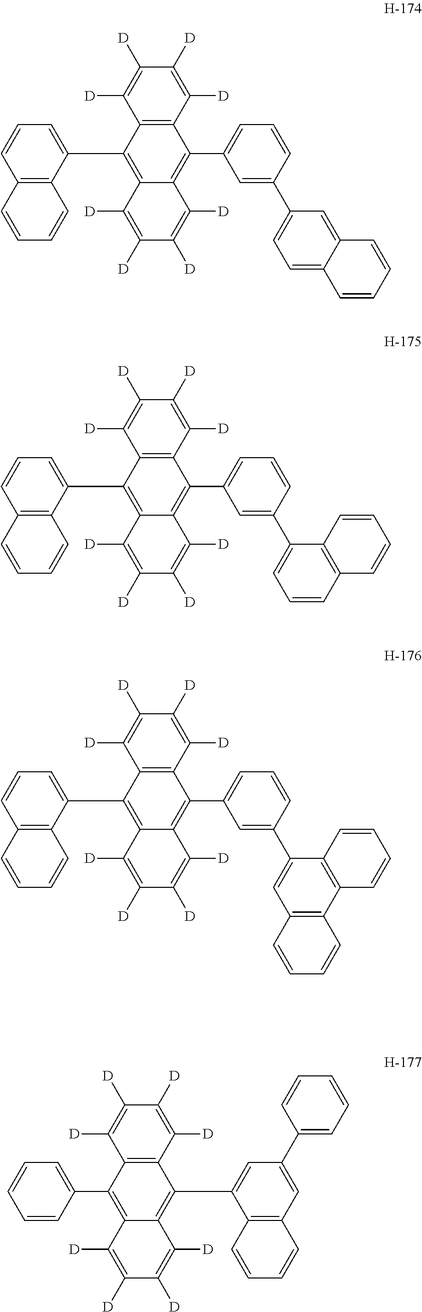 [SMILES: [2H]c1c([2H])c([2H])c2c(-c3cccc4ccccc34)c3c([2H])c([2H])c([2H])c([2H])c3c(-c3cccc(-c4ccc5ccccc5c4)c3)c2c1[2H] ; [2H]c1c([2H])c([2H])c2c(-c3cccc4ccccc34)c3c([2H])c([2H])c([2H])c([2H])c3c(-c3cccc(-c4cccc5ccccc45)c3)c2c1[2H] ; [2H]c1c([2H])c([2H])c2c(-c3cccc4ccccc34)c3c([2H])c([2H])c([2H])c([2H])c3c(-c3cccc(-c4cc5ccccc5c5ccccc45)c3)c2c1[2H] ; [2H]c1c([2H])c([2H])c2c(-c3cc(-c4ccccc4)cc4ccccc34)c3c([2H])c([2H])c([2H])c([2H])c3c(-c3ccccc3)c2c1[2H]]

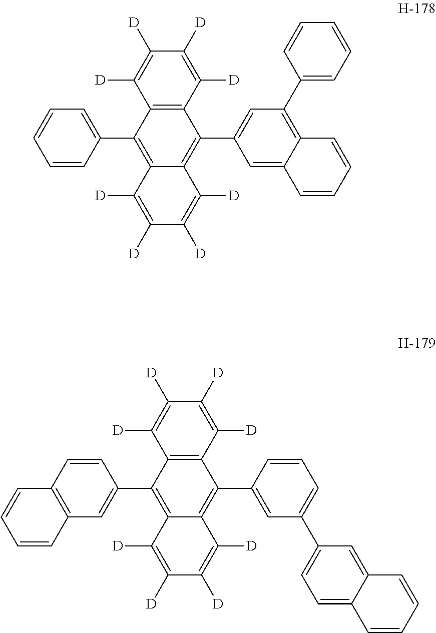 [SMILES: [2H]c1c([2H])c([2H])c2c(-c3cc(-c4ccccc4)c4ccccc4c3)c3c([2H])c([2H])c([2H])c([2H])c3c(-c3ccccc3)c2c1[2H] ; [2H]c1c([2H])c([2H])c2c(-c3ccc4ccccc4c3)c3c([2H])c([2H])c([2H])c([2H])c3c(-c3cccc(-c4ccc5ccccc5c4)c3)c2c1[2H]]

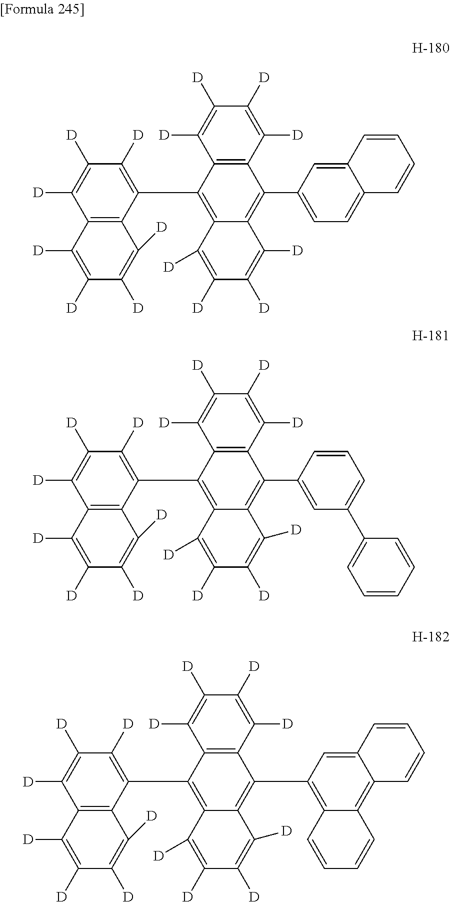 [SMILES: [2H]c1c([2H])c([2H])c2c(-c3c4c([2H])c([2H])c([2H])c([2H])c4c(-c4ccc5ccccc5c4)c4c([2H])c([2H])c([2H])c([2H])c34)c([2H])c([2H])c([2H])c2c1[2H] ; [2H]c1c([2H])c([2H])c2c(-c3c4c([2H])c([2H])c([2H])c([2H])c4c(-c4cccc(-c5ccccc5)c4)c4c([2H])c([2H])c([2H])c([2H])c34)c([2H])c([2H])c([2H])c2c1[2H] ; [2H]c1c([2H])c([2H])c2c(-c3c4c([2H])c([2H])c([2H])c([2H])c4c(-c4cc5ccccc5c5ccccc45)c4c([2H])c([2H])c([2H])c([2H])c34)c([2H])c([2H])c([2H])c2c1[2H]]

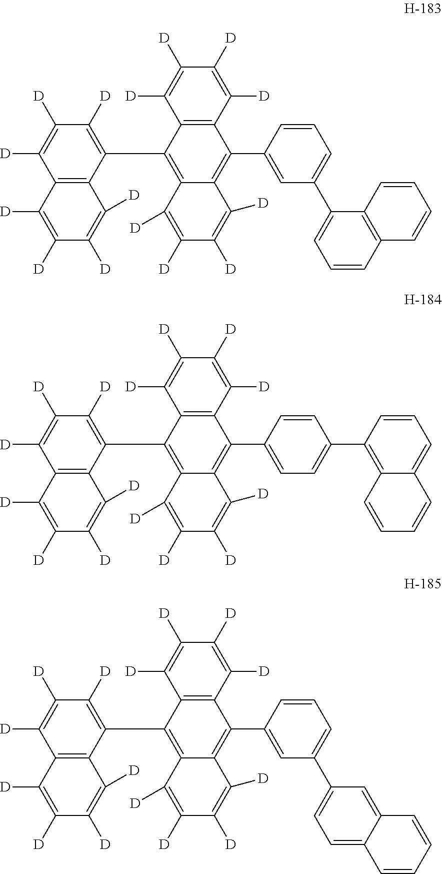 [SMILES: [2H]c1c([2H])c([2H])c2c(-c3c4c([2H])c([2H])c([2H])c([2H])c4c(-c4cccc(-c5cccc6ccccc56)c4)c4c([2H])c([2H])c([2H])c([2H])c34)c([2H])c([2H])c([2H])c2c1[2H] ; [2H]c1c([2H])c([2H])c2c(-c3c4c([2H])c([2H])c([2H])c([2H])c4c(-c4ccc(-c5cccc6ccccc56)cc4)c4c([2H])c([2H])c([2H])c([2H])c34)c([2H])c([2H])c([2H])c2c1[2H] ; [2H]c1c([2H])c([2H])c2c(-c3c4c([2H])c([2H])c([2H])c([2H])c4c(-c4cccc(-c5ccc6ccccc6c5)c4)c4c([2H])c([2H])c([2H])c([2H])c34)c([2H])c([2H])c([2H])c2c1[2H]]

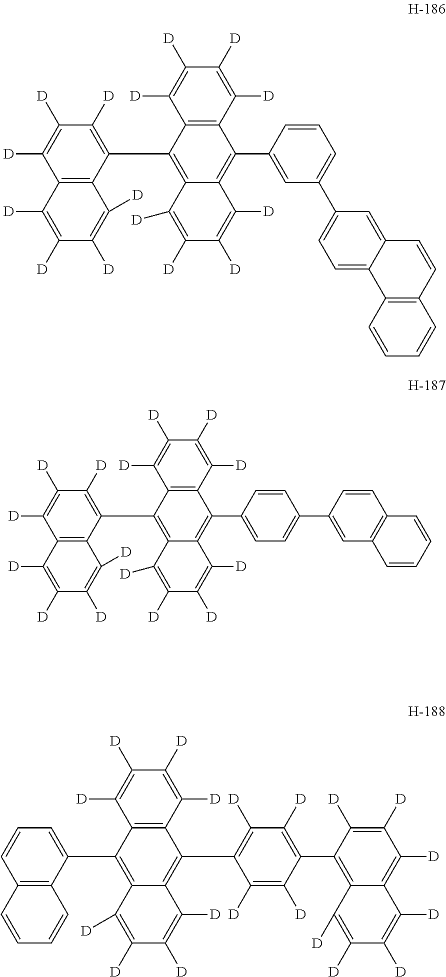 [SMILES: [2H]c1c([2H])c([2H])c2c(-c3c4c([2H])c([2H])c([2H])c([2H])c4c(-c4cccc(-c5ccc6c(ccc7ccccc76)c5)c4)c4c([2H])c([2H])c([2H])c([2H])c34)c([2H])c([2H])c([2H])c2c1[2H] ; [2H]c1c([2H])c([2H])c2c(-c3c4c([2H])c([2H])c([2H])c([2H])c4c(-c4ccc(-c5ccc6ccccc6c5)cc4)c4c([2H])c([2H])c([2H])c([2H])c34)c([2H])c([2H])c([2H])c2c1[2H] ; [2H]c1c([2H])c(-c2c3c([2H])c([2H])c([2H])c([2H])c3c(-c3cccc4ccccc34)c3c([2H])c([2H])c([2H])c([2H])c23)c([2H])c([2H])c1-c1c([2H])c([2H])c([2H])c2c([2H])c([2H])c([2H])c([2H])c12]

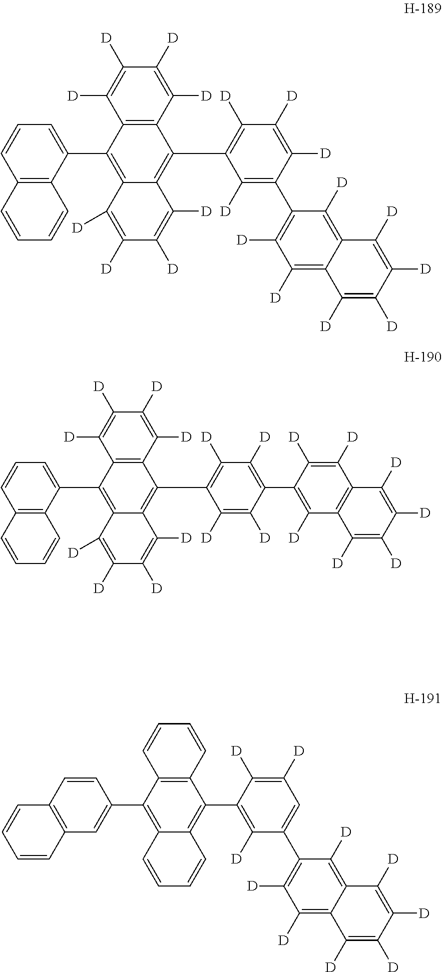 [SMILES: [2H]c1c([2H])c(-c2c([2H])c([2H])c3c([2H])c([2H])c([2H])c([2H])c3c2[2H])c([2H])c(-c2c3c([2H])c([2H])c([2H])c([2H])c3c(-c3cccc4ccccc34)c3c([2H])c([2H])c([2H])c([2H])c23)c1[2H] ; [2H]c1c([2H])c(-c2c3c([2H])c([2H])c([2H])c([2H])c3c(-c3cccc4ccccc34)c3c([2H])c([2H])c([2H])c([2H])c23)c([2H])c([2H])c1-c1c([2H])c([2H])c2c([2H])c([2H])c([2H])c([2H])c2c1[2H] ; [2H]c1cc(-c2c([2H])c([2H])c3c([2H])c([2H])c([2H])c([2H])c3c2[2H])c([2H])c(-c2c3ccccc3c(-c3ccc4ccccc4c3)c3ccccc23)c1[2H]]

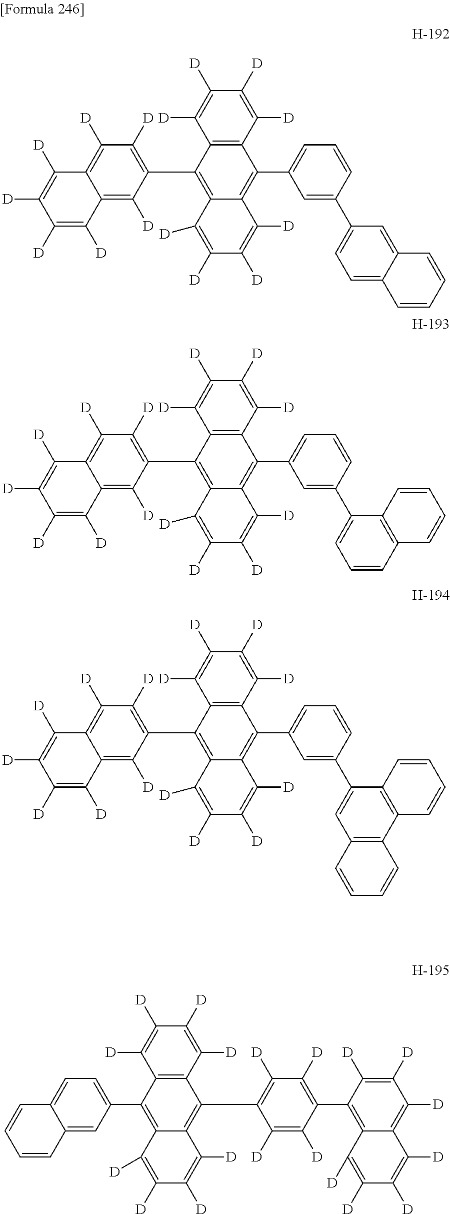

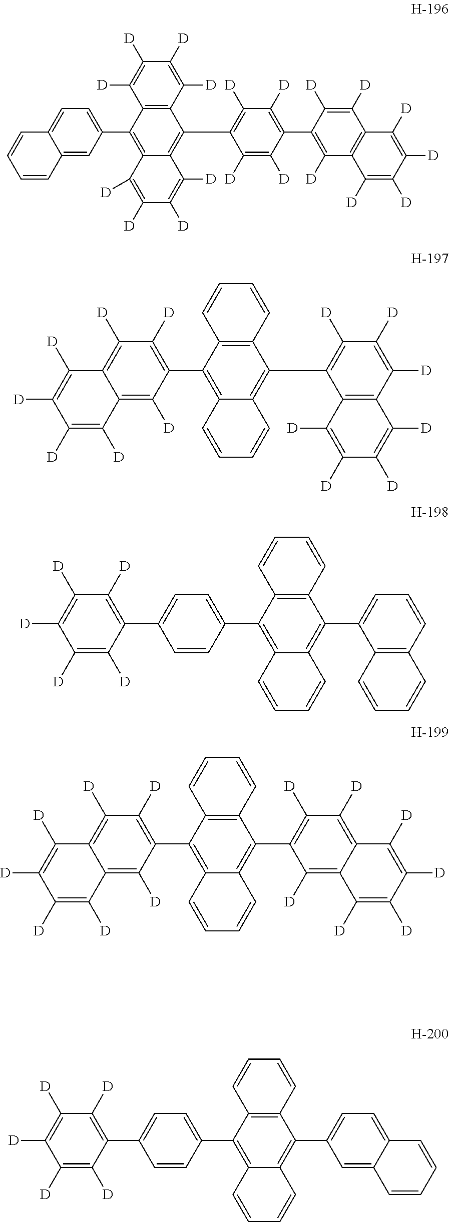

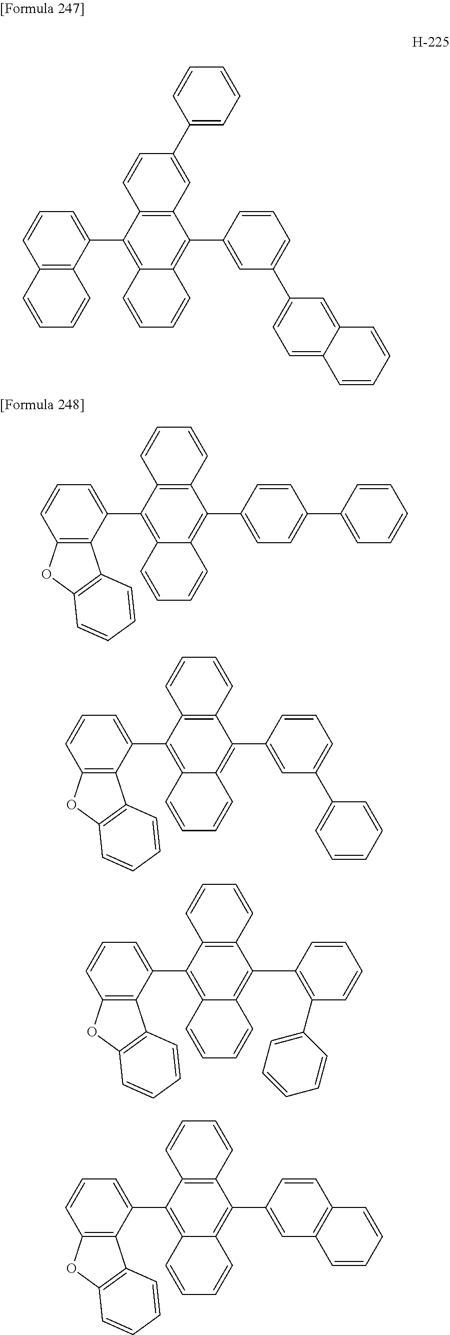

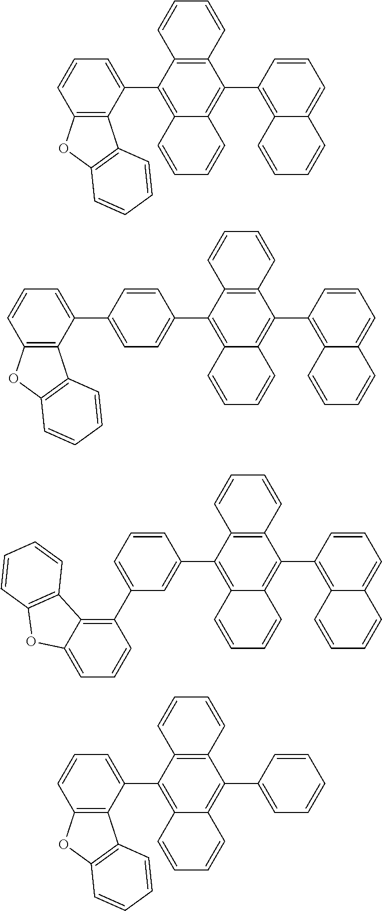 [SMILES: c1ccc2c(-c3c4ccccc4c(-c4cccc5oc6ccccc6c45)c4ccccc34)cccc2c1 ; c1ccc2c(-c3c4ccccc4c(-c4ccc(-c5cccc6oc7ccccc7c56)cc4)c4ccccc34)cccc2c1 ; c1cc(-c2c3ccccc3c(-c3cccc4ccccc34)c3ccccc23)cc(-c2cccc3oc4ccccc4c23)c1 ; c1ccc(-c2c3ccccc3c(-c3cccc4oc5ccccc5c34)c3ccccc23)cc1]

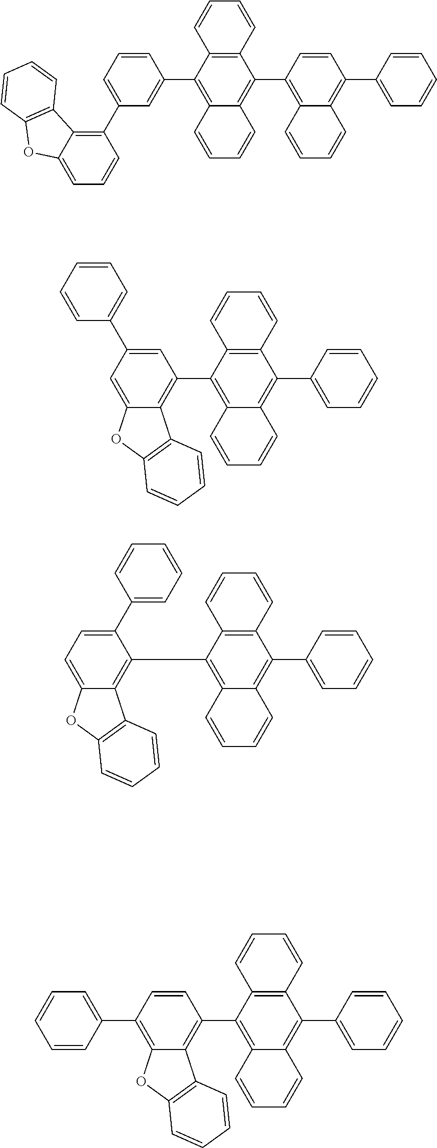

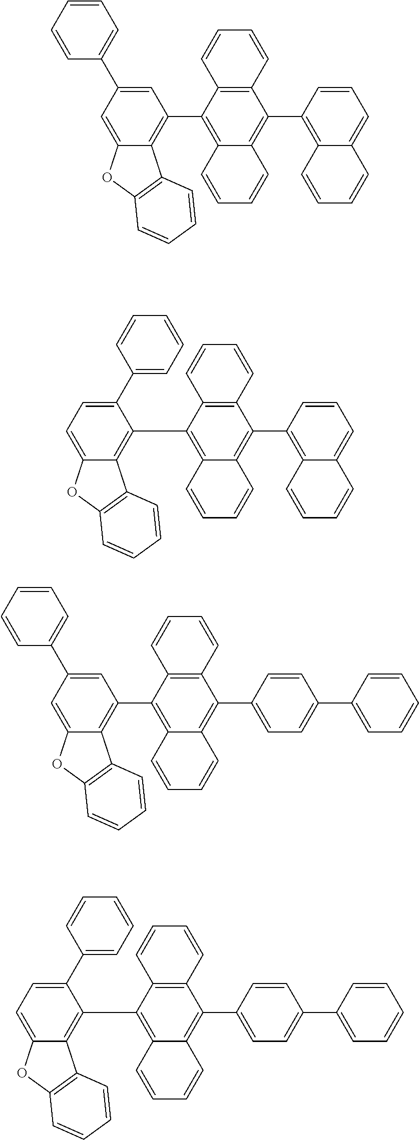 [SMILES: c1ccc(-c2cc(-c3c4ccccc4c(-c4cccc5ccccc45)c4ccccc34)c3c(c2)oc2ccccc23)cc1 ; c1ccc(-c2ccc3oc4ccccc4c3c2-c2c3ccccc3c(-c3cccc4ccccc34)c3ccccc23)cc1 ; c1ccc(-c2ccc(-c3c4ccccc4c(-c4cc(-c5ccccc5)cc5oc6ccccc6c45)c4ccccc34)cc2)cc1 ; c1ccc(-c2ccc(-c3c4ccccc4c(-c4c(-c5ccccc5)ccc5oc6ccccc6c45)c4ccccc34)cc2)cc1]

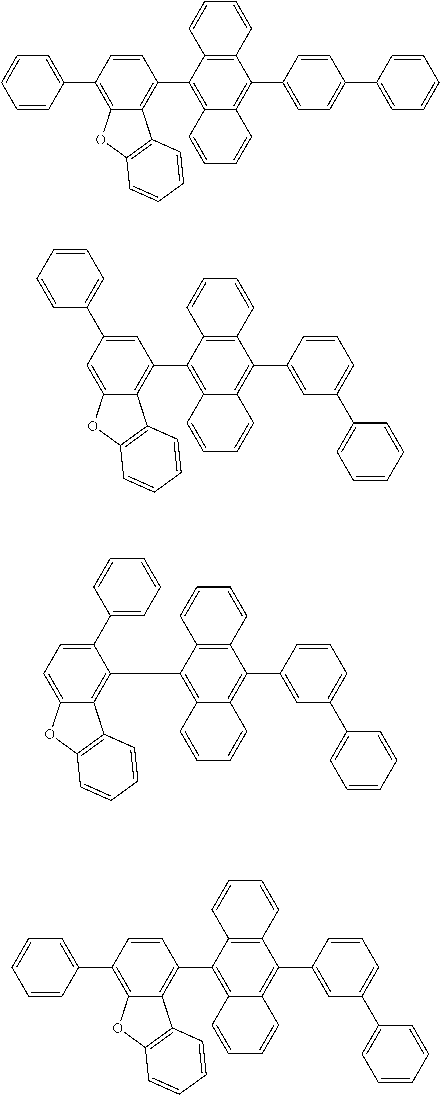 [SMILES: c1ccc(-c2ccc(-c3c4ccccc4c(-c4ccc(-c5ccccc5)c5oc6ccccc6c45)c4ccccc34)cc2)cc1 ; c1ccc(-c2cccc(-c3c4ccccc4c(-c4cc(-c5ccccc5)cc5oc6ccccc6c45)c4ccccc34)c2)cc1 ; c1ccc(-c2cccc(-c3c4ccccc4c(-c4c(-c5ccccc5)ccc5oc6ccccc6c45)c4ccccc34)c2)cc1 ; c1ccc(-c2cccc(-c3c4ccccc4c(-c4ccc(-c5ccccc5)c5oc6ccccc6c45)c4ccccc34)c2)cc1]

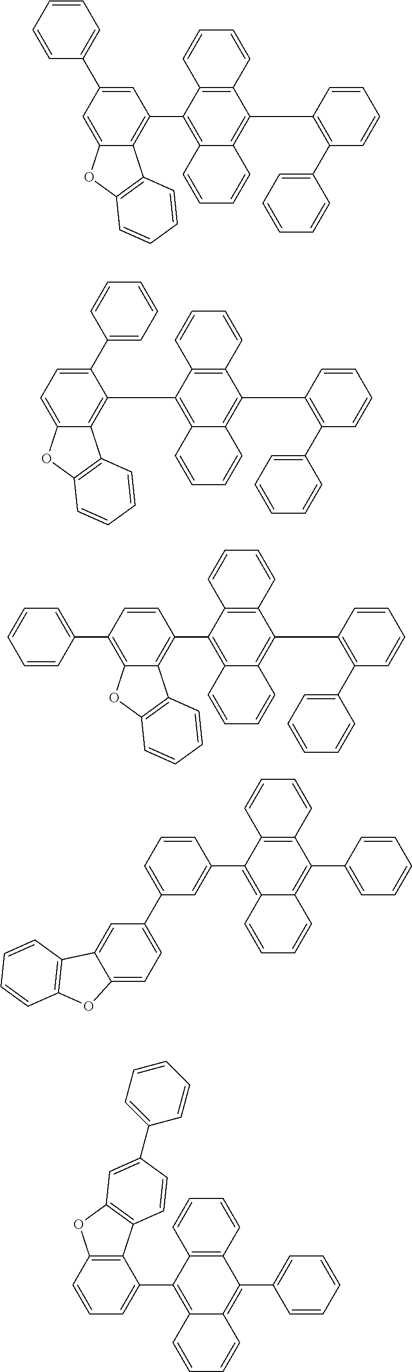

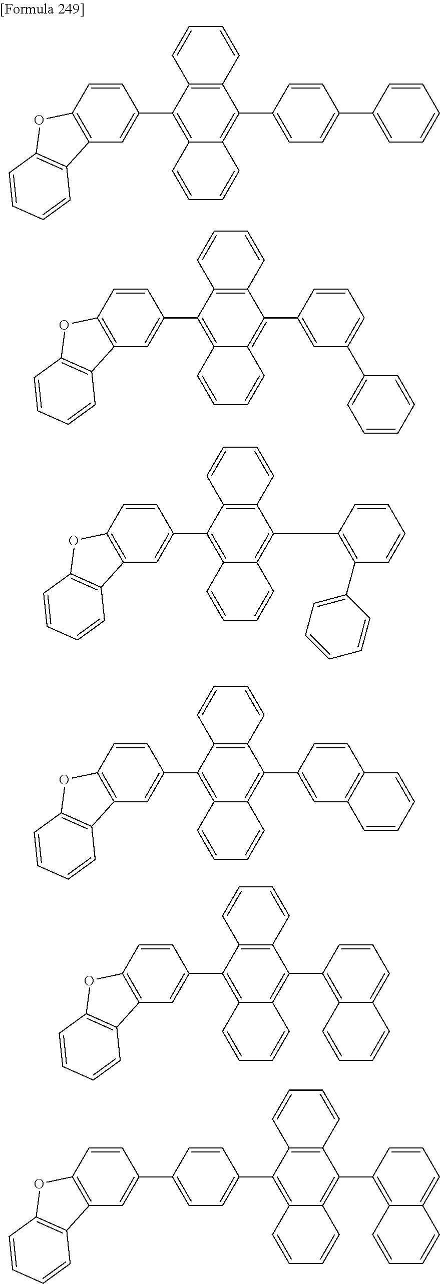

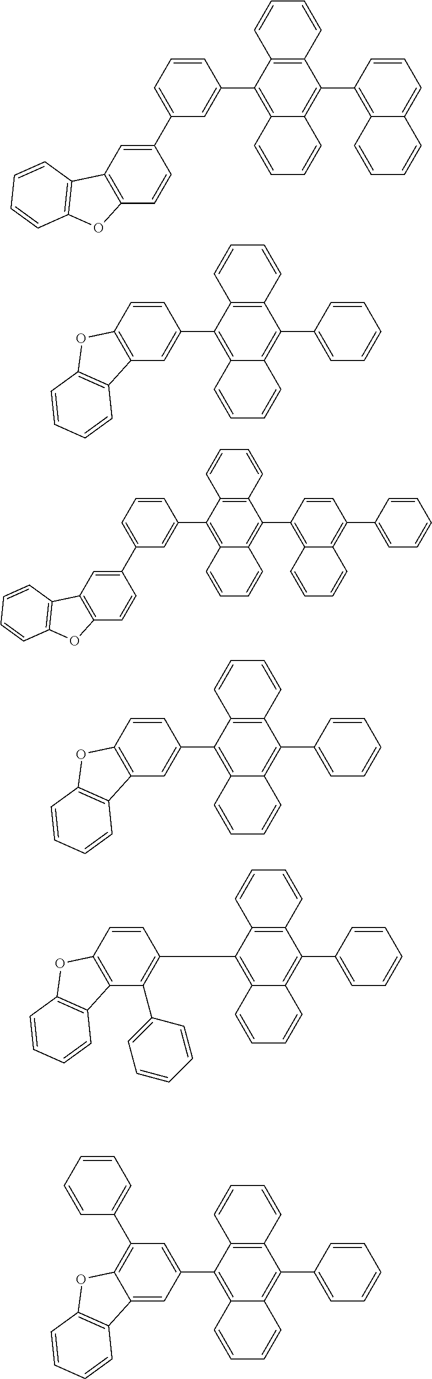

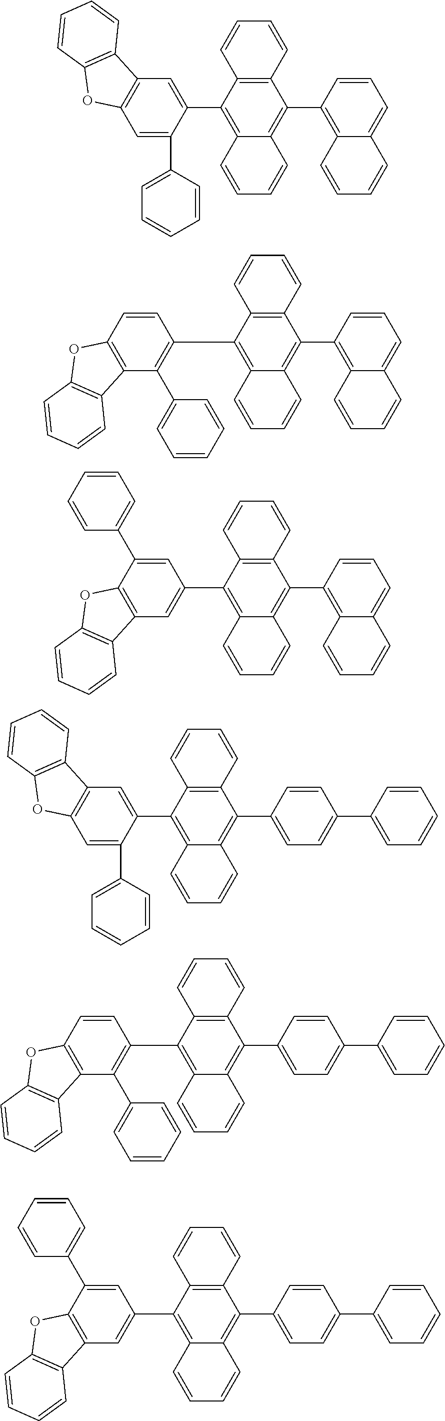

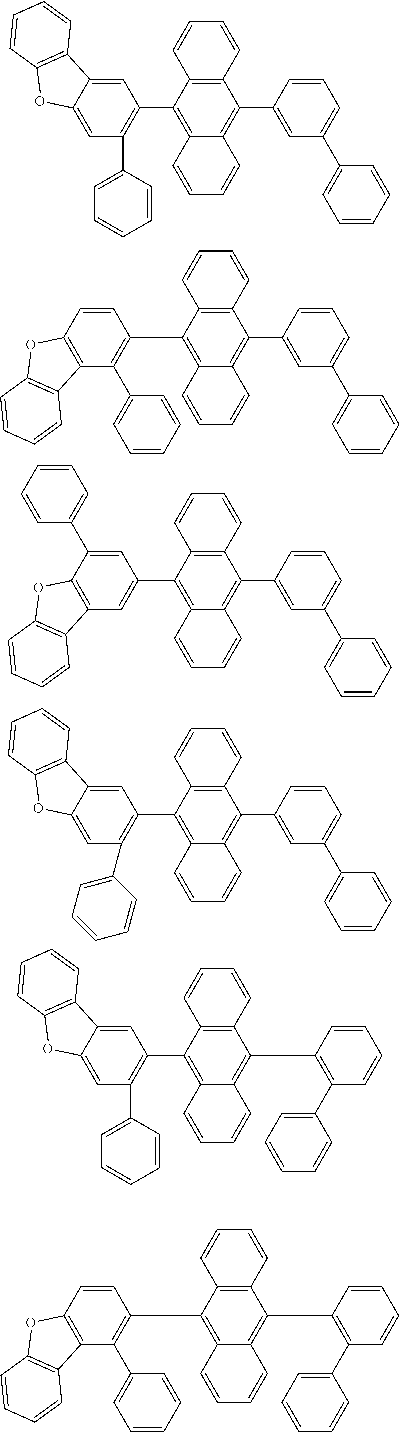

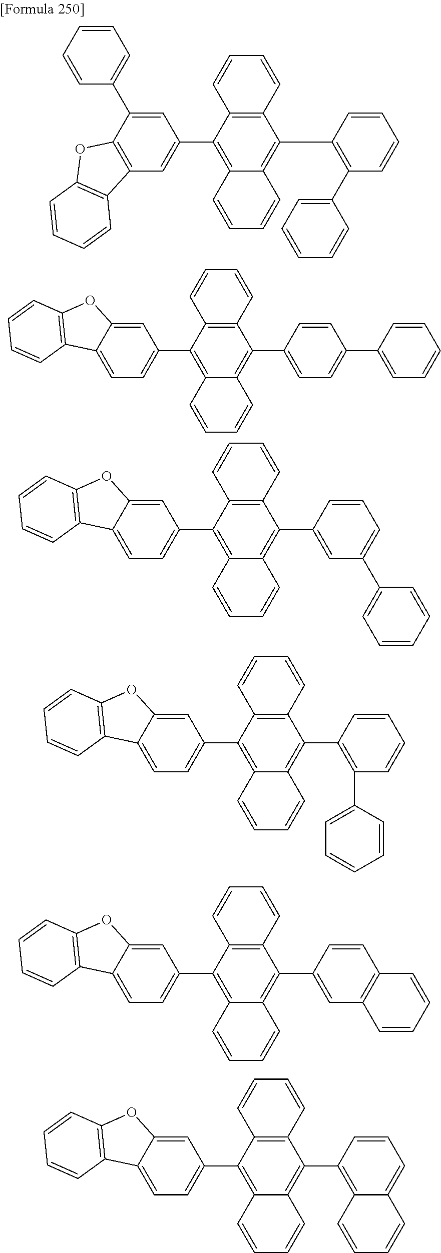

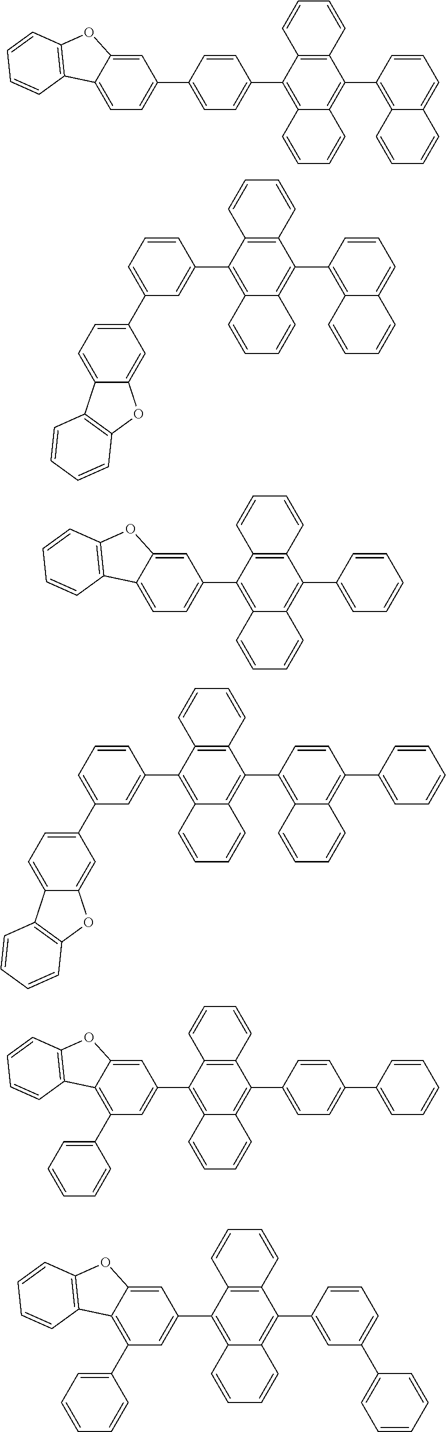

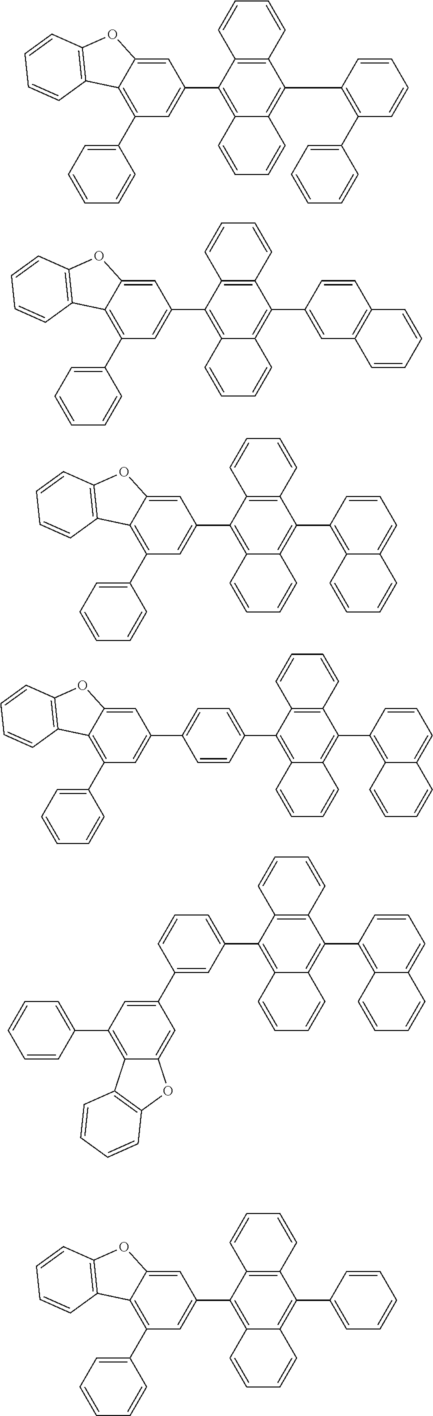

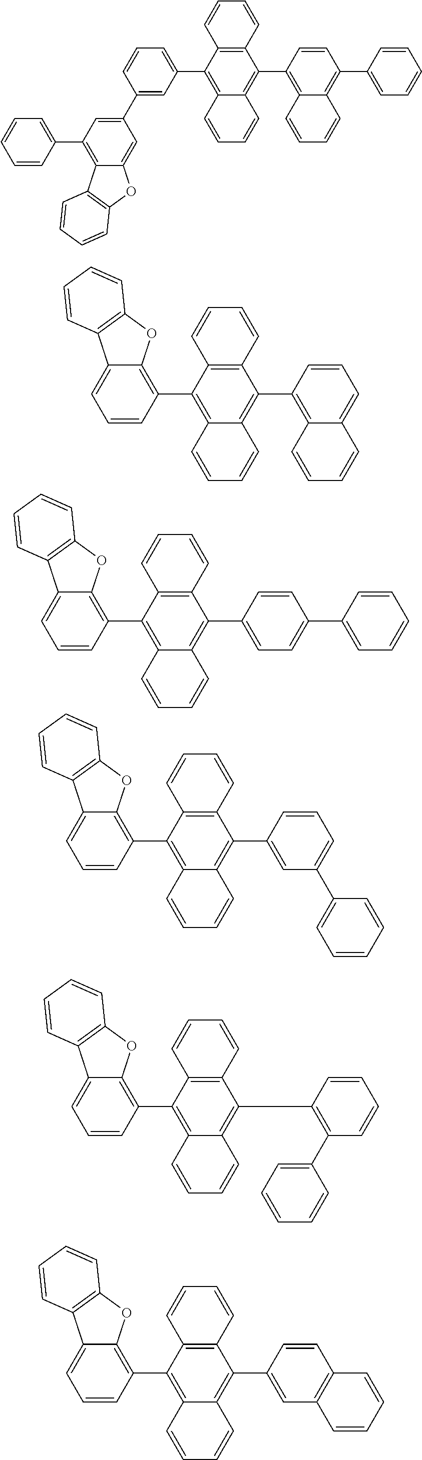

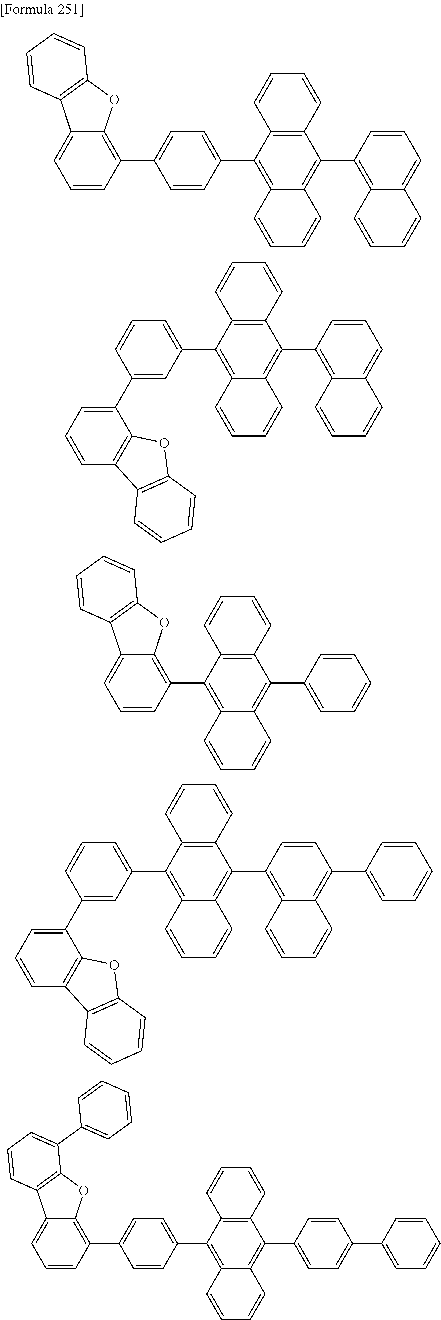

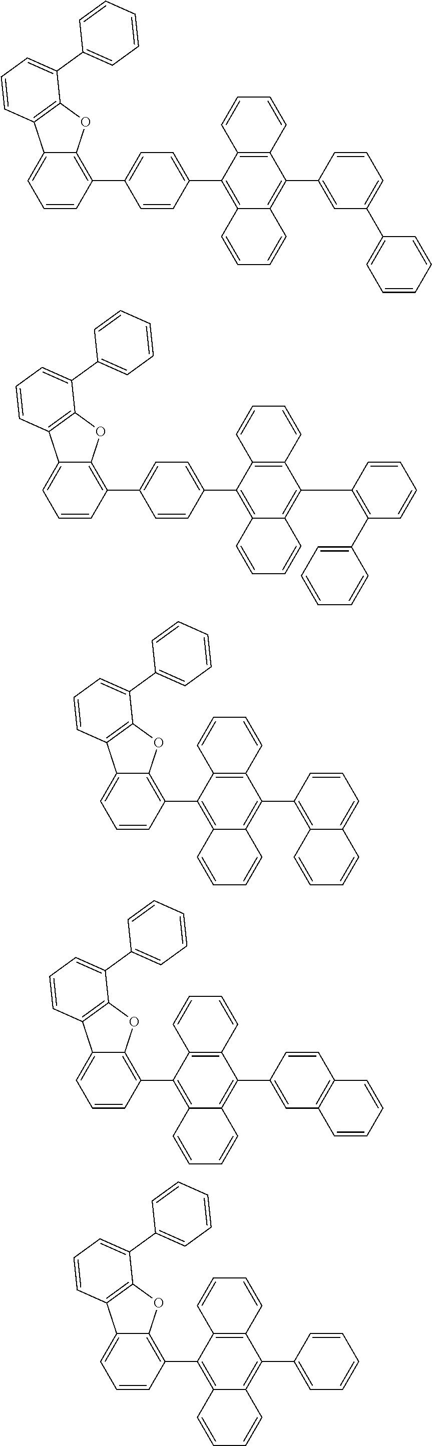

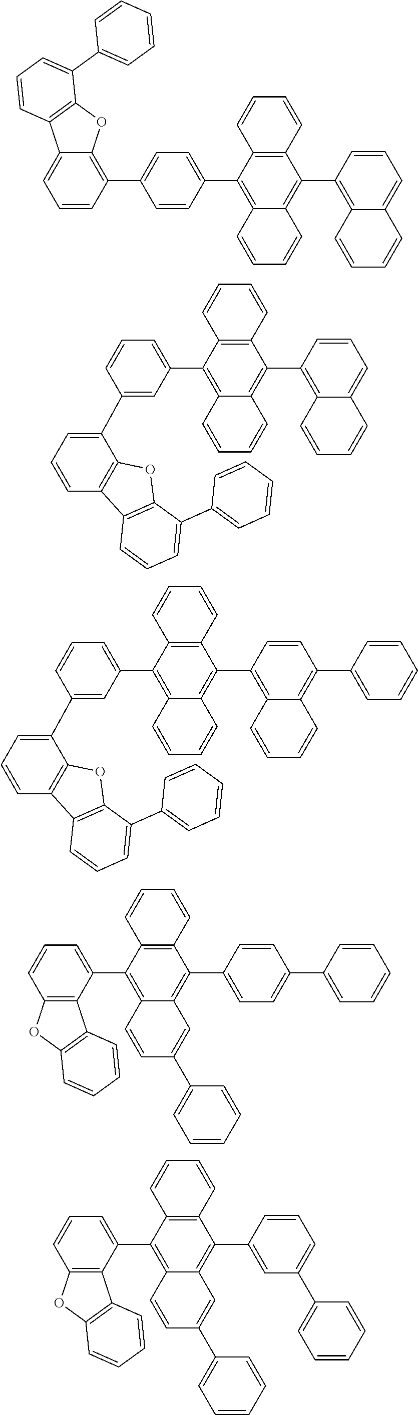

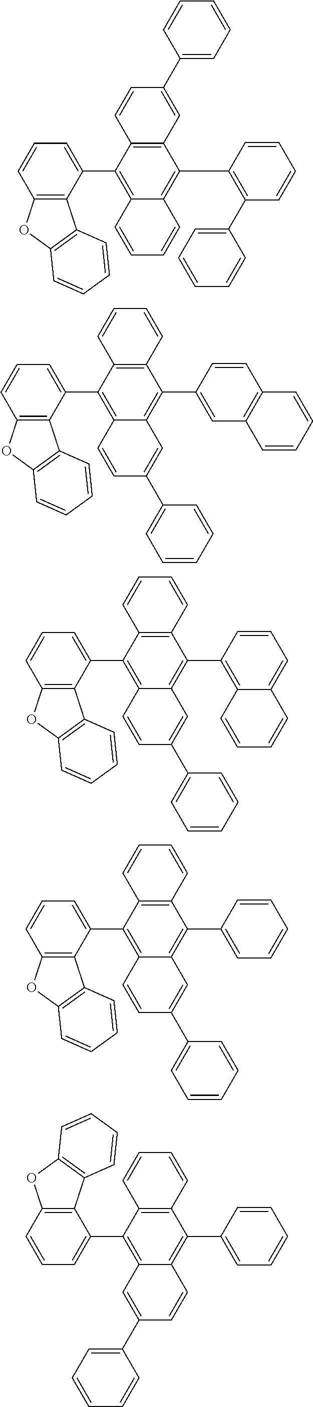

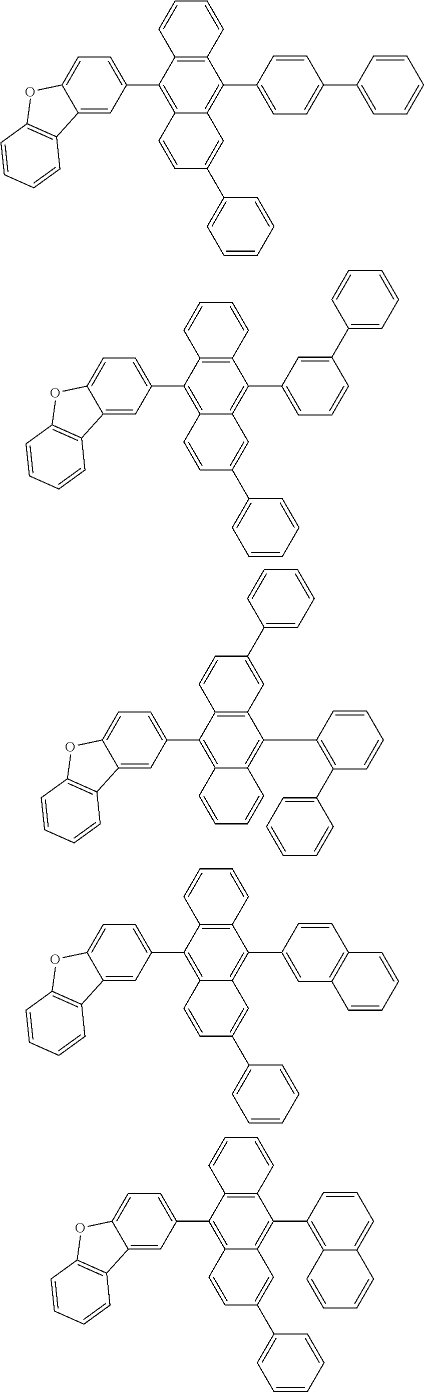

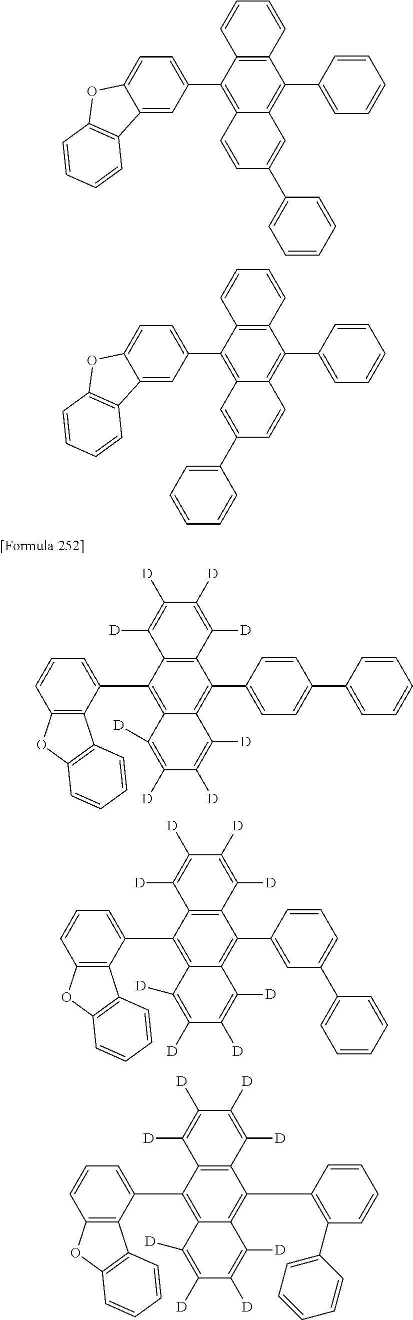

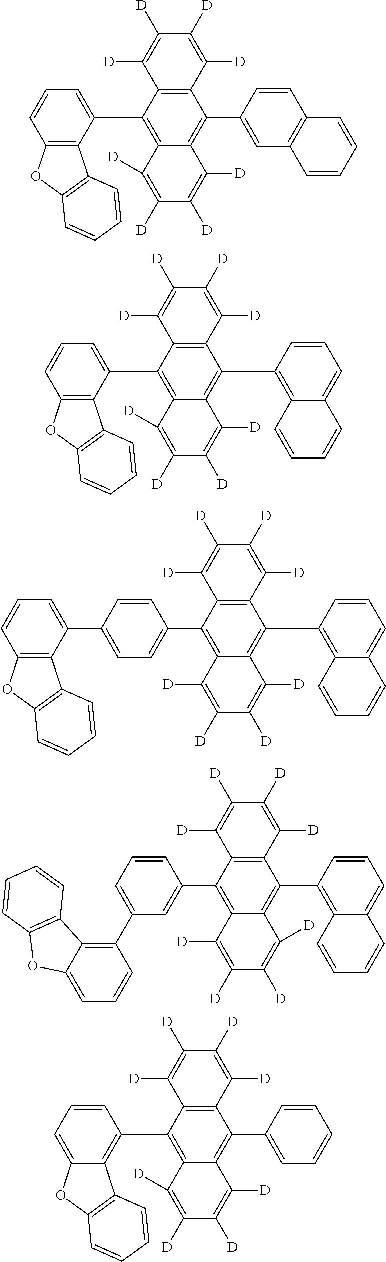

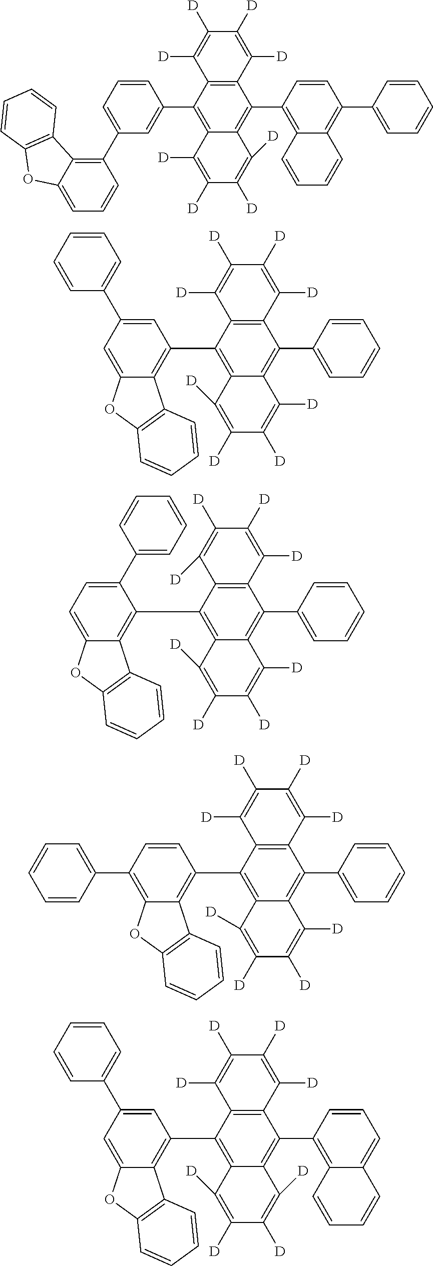

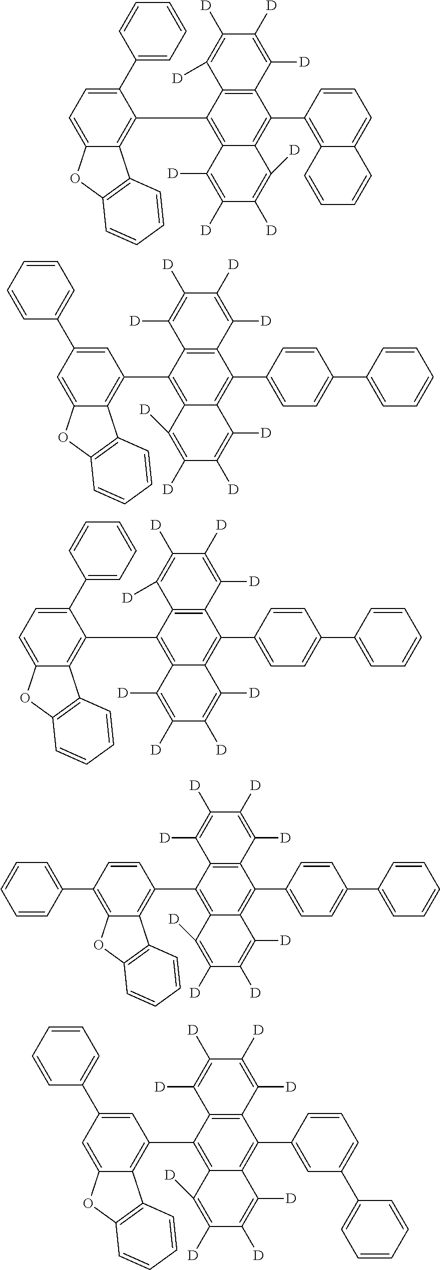

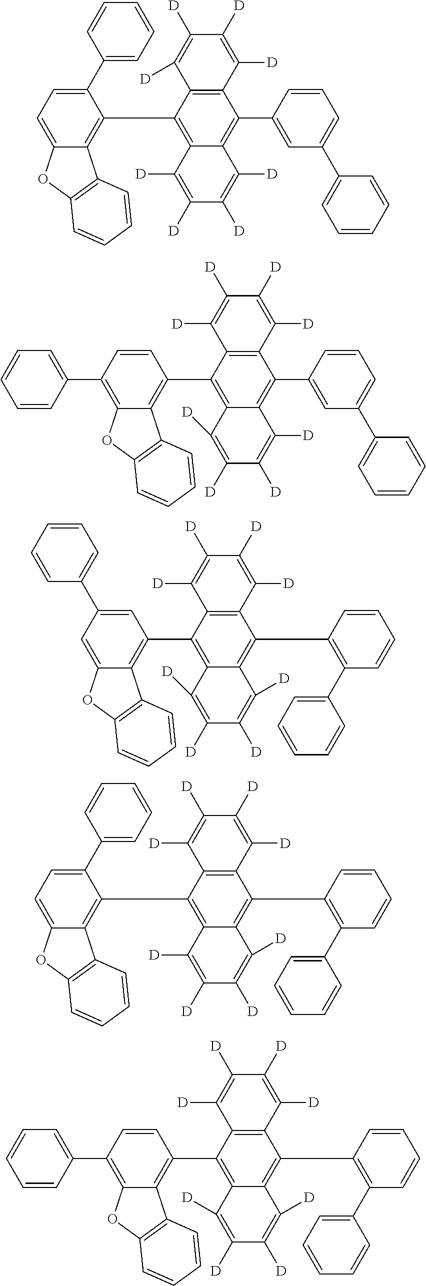

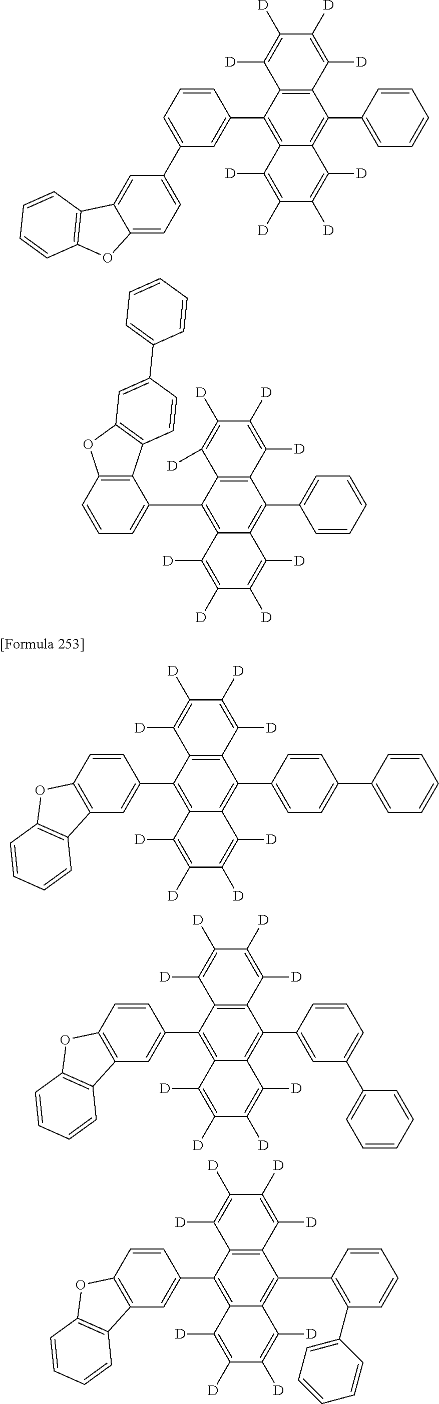

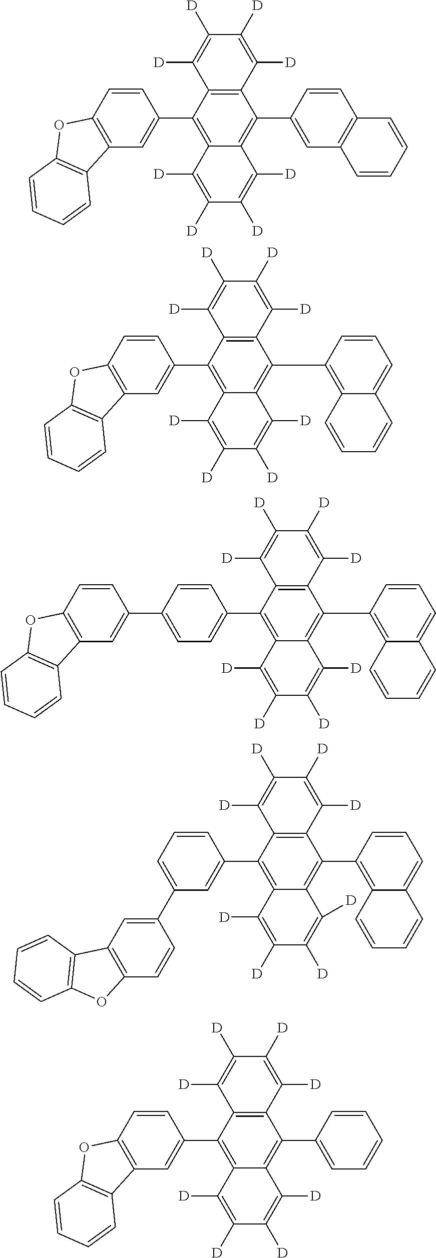

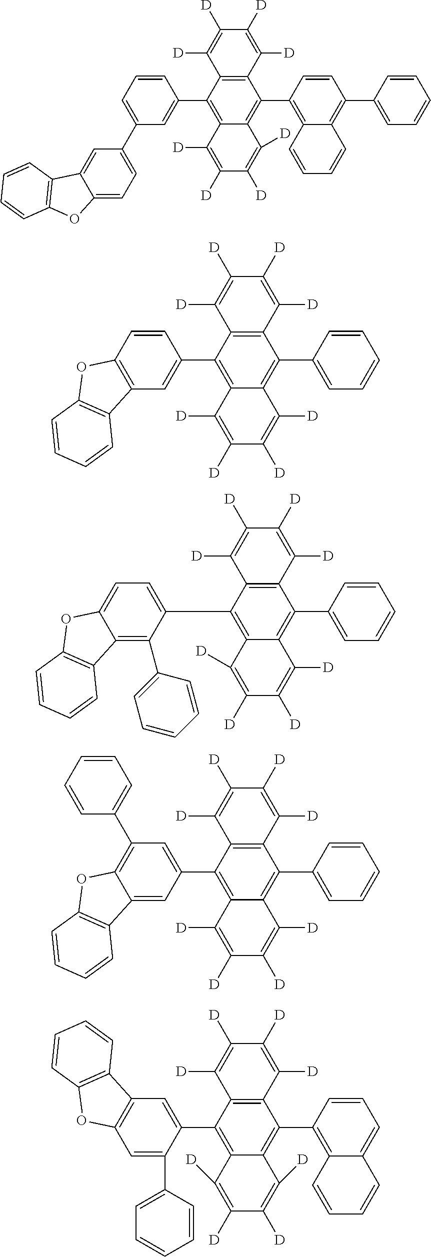

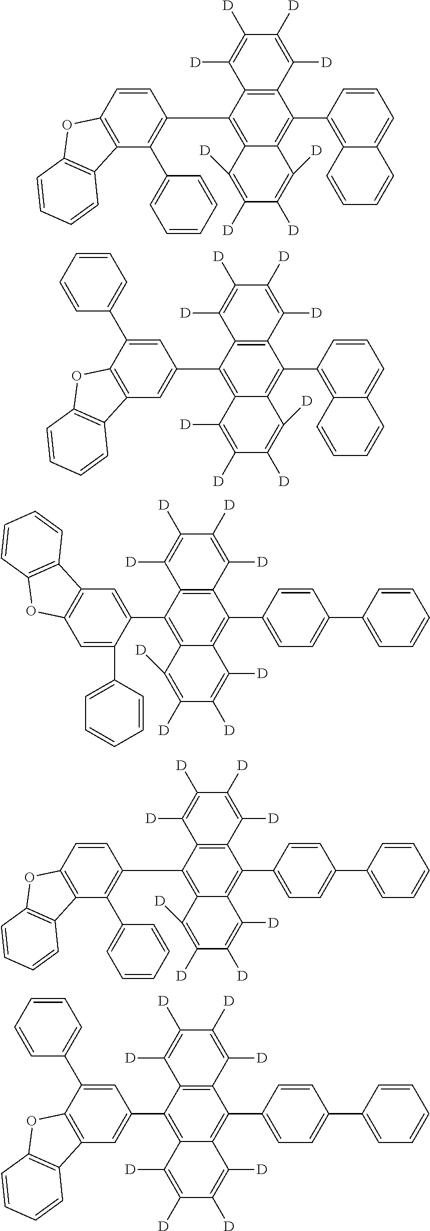

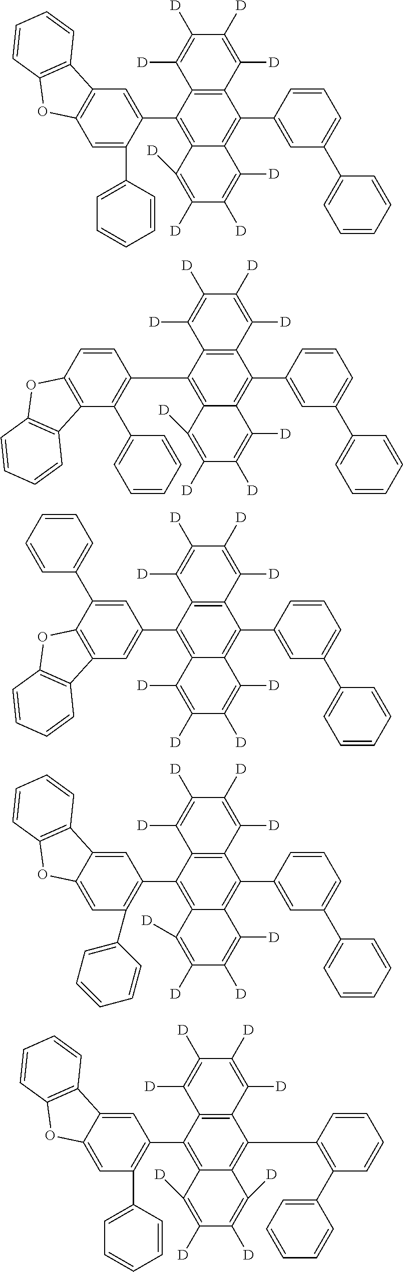

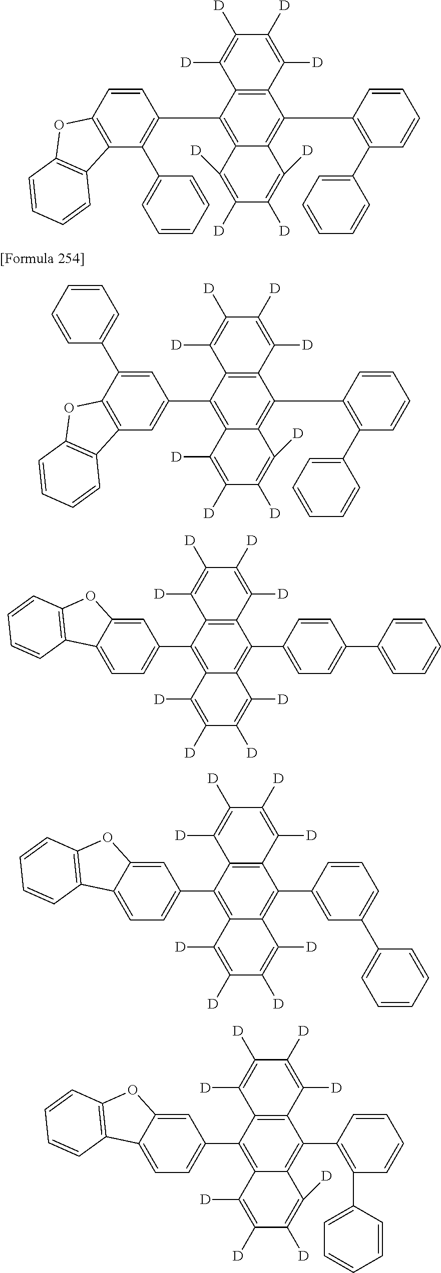

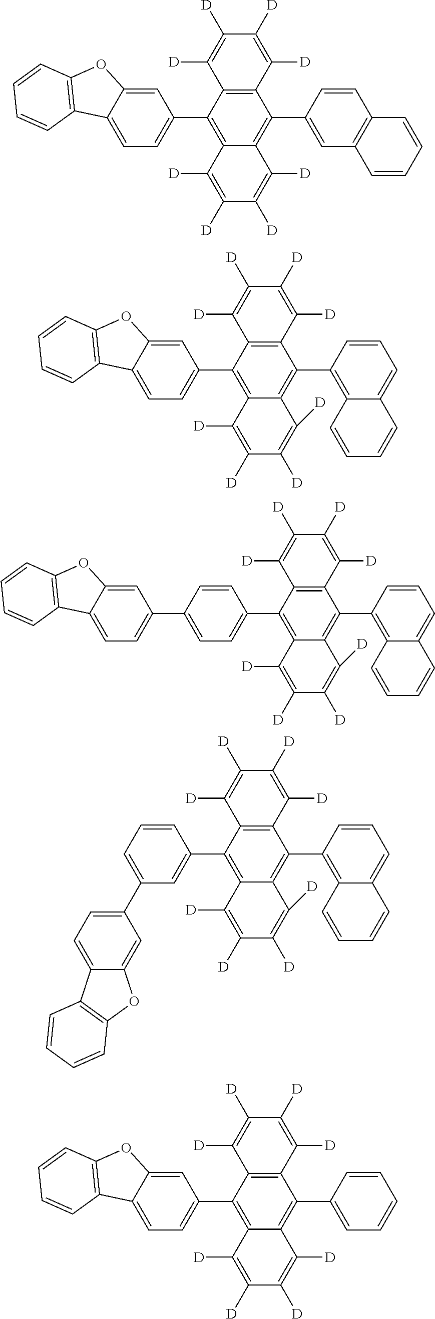

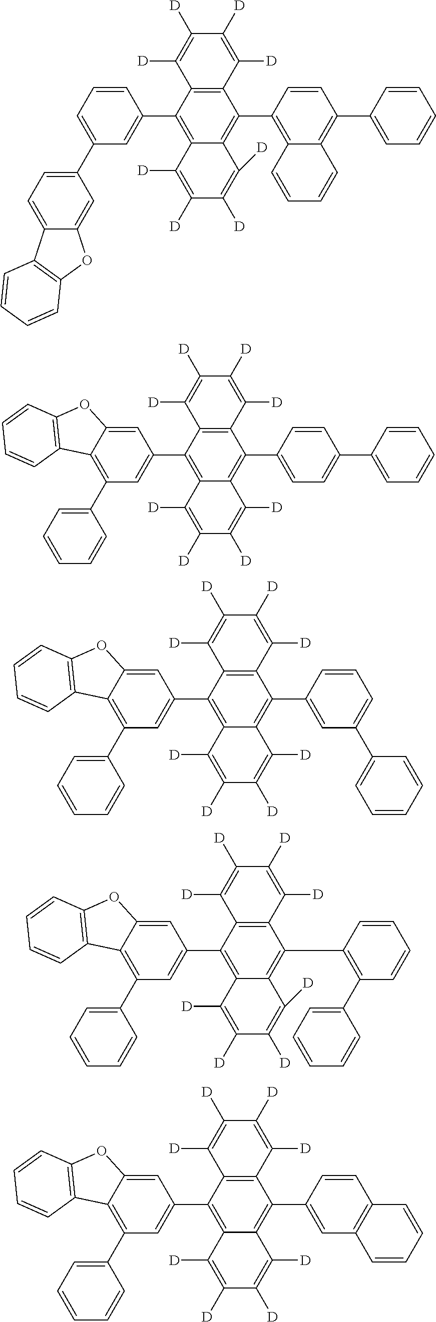

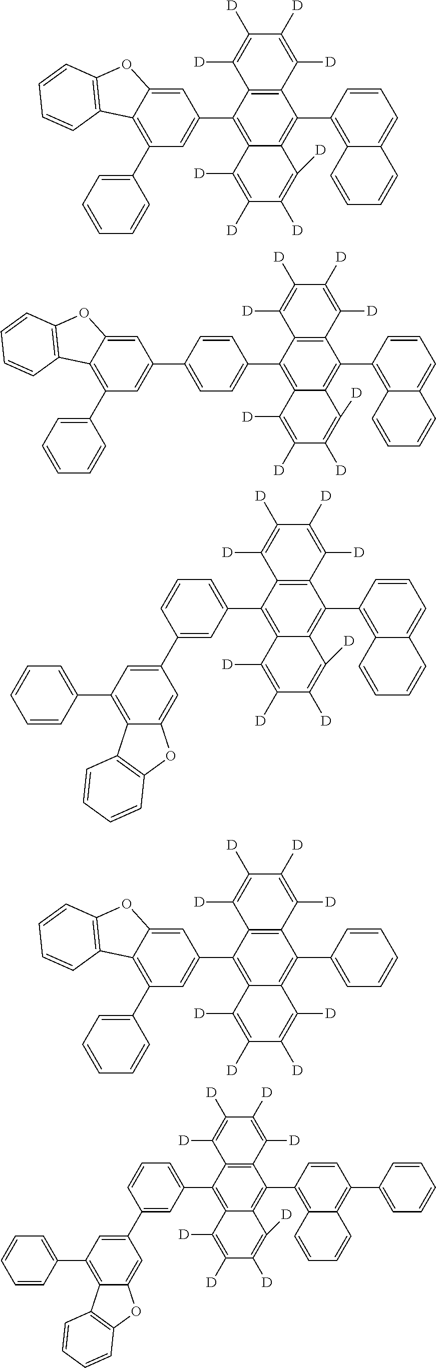

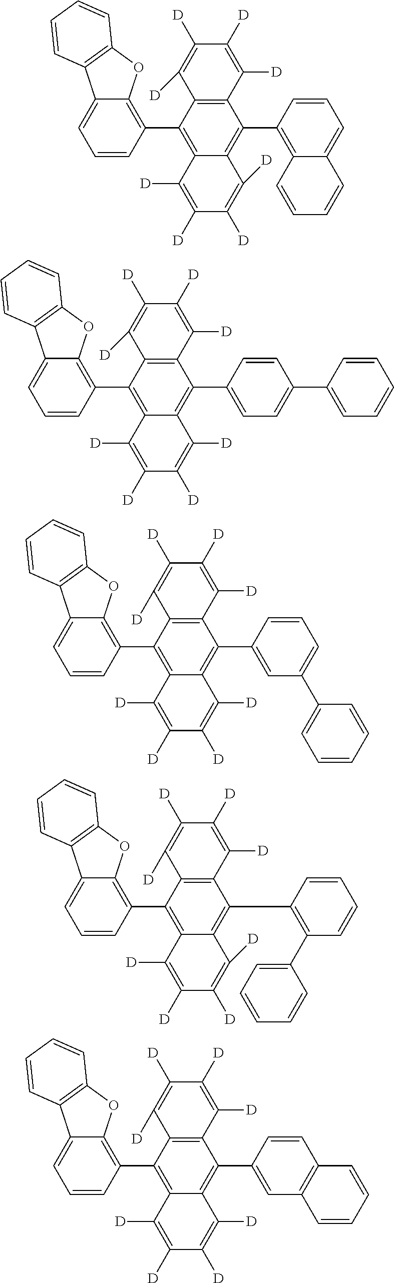

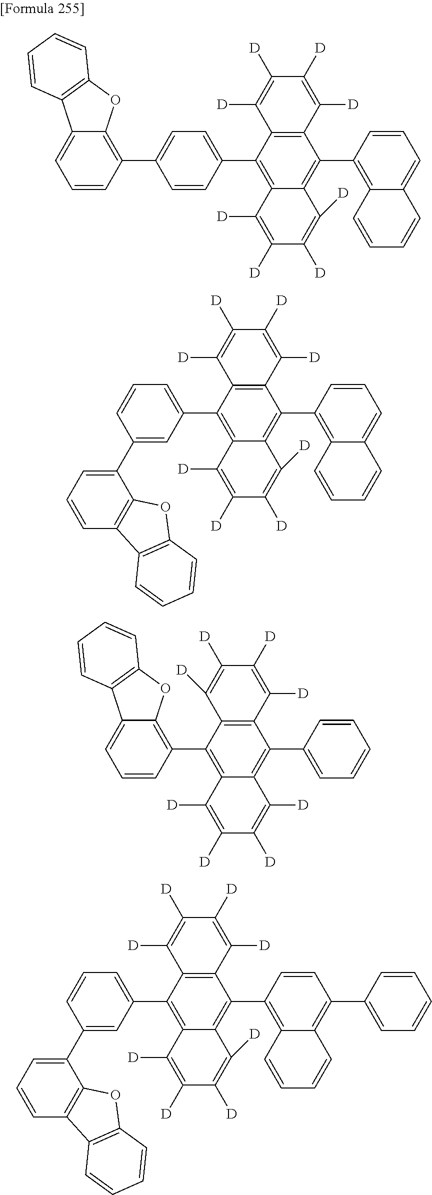

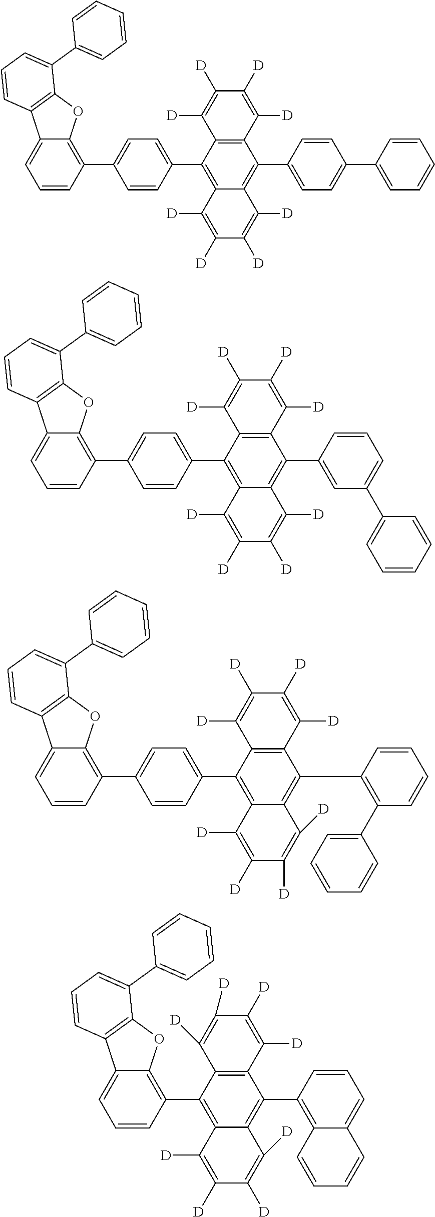

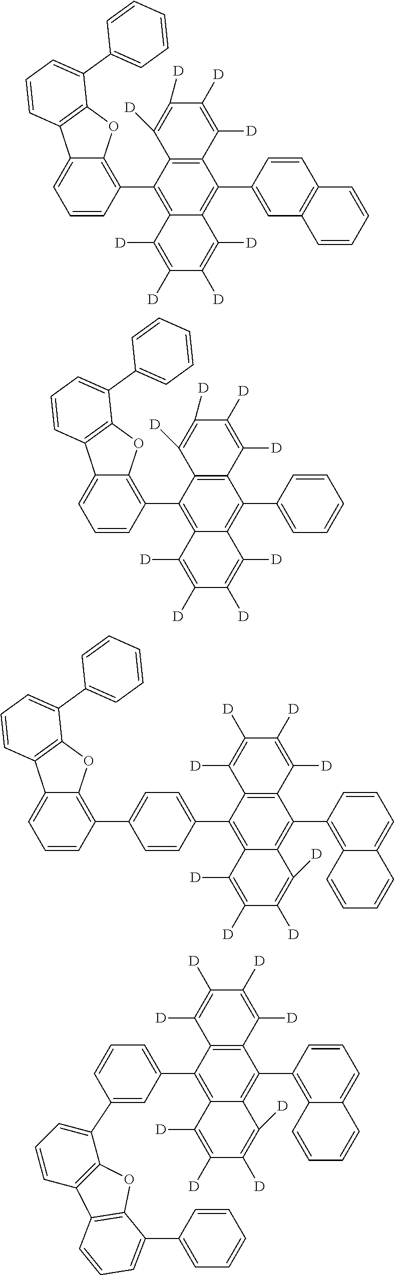

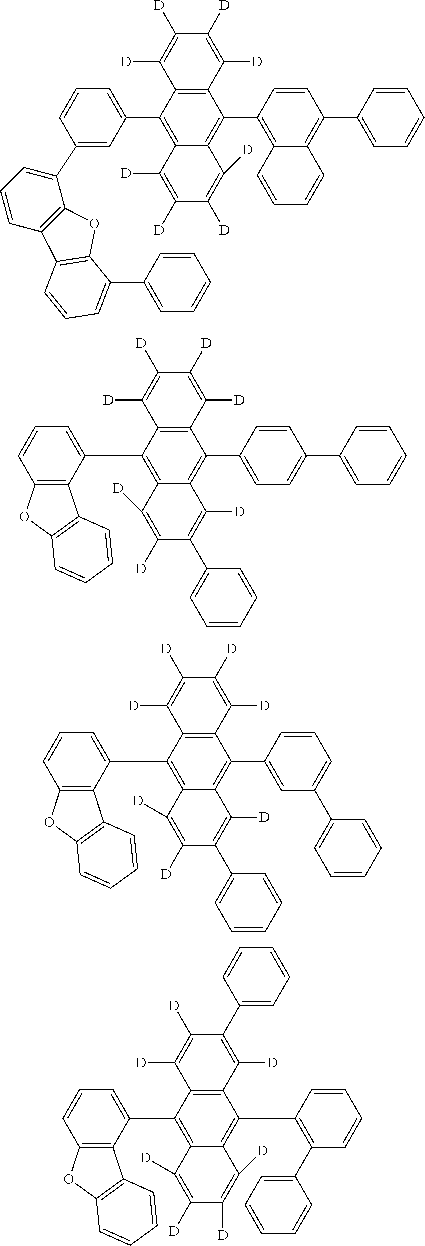

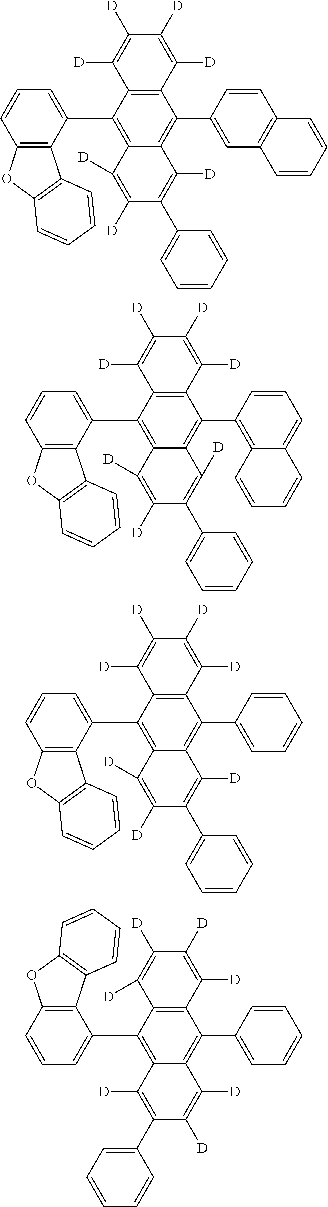

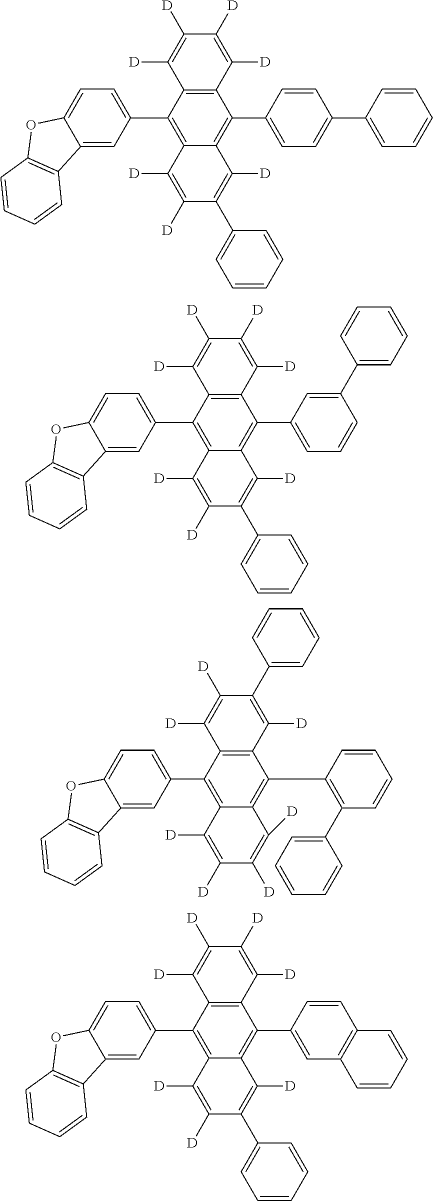

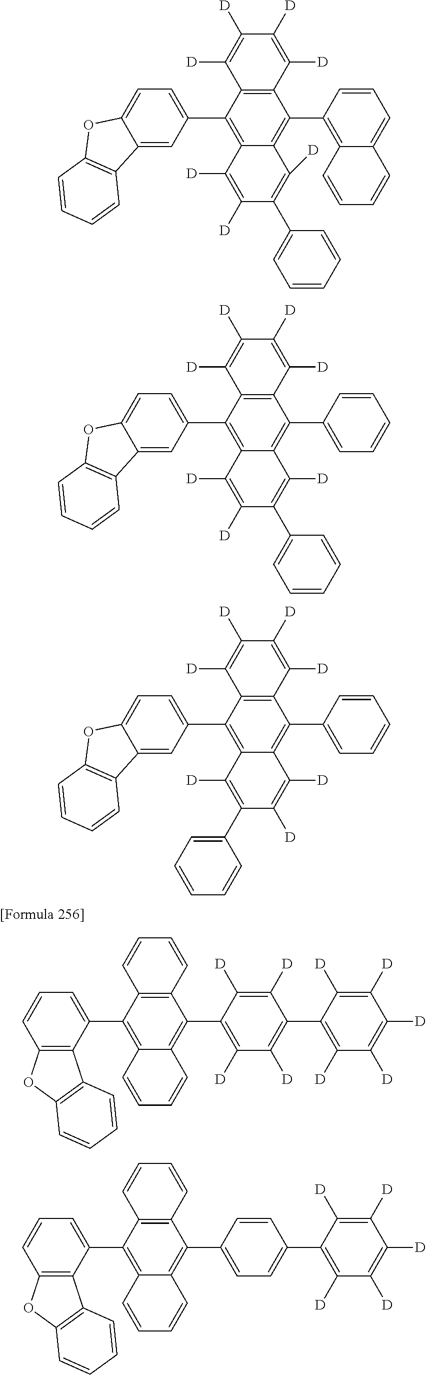

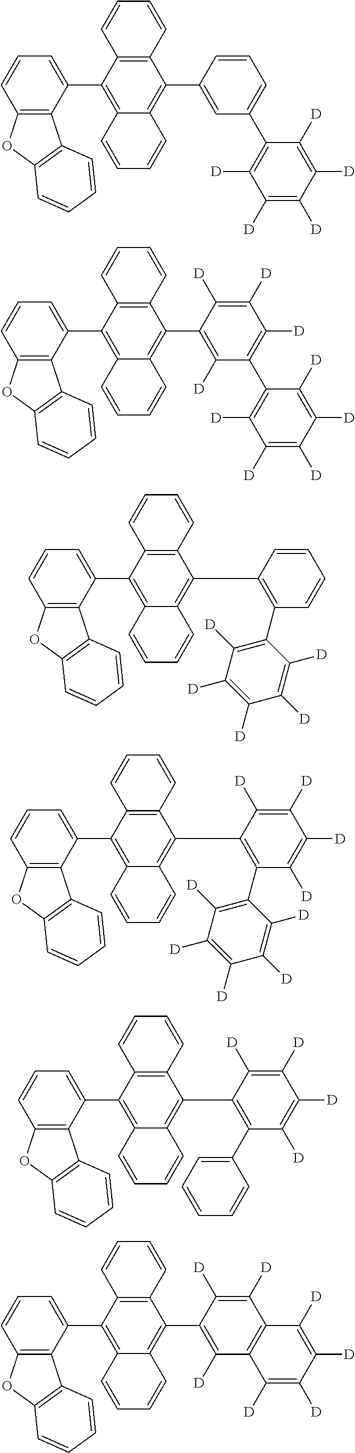

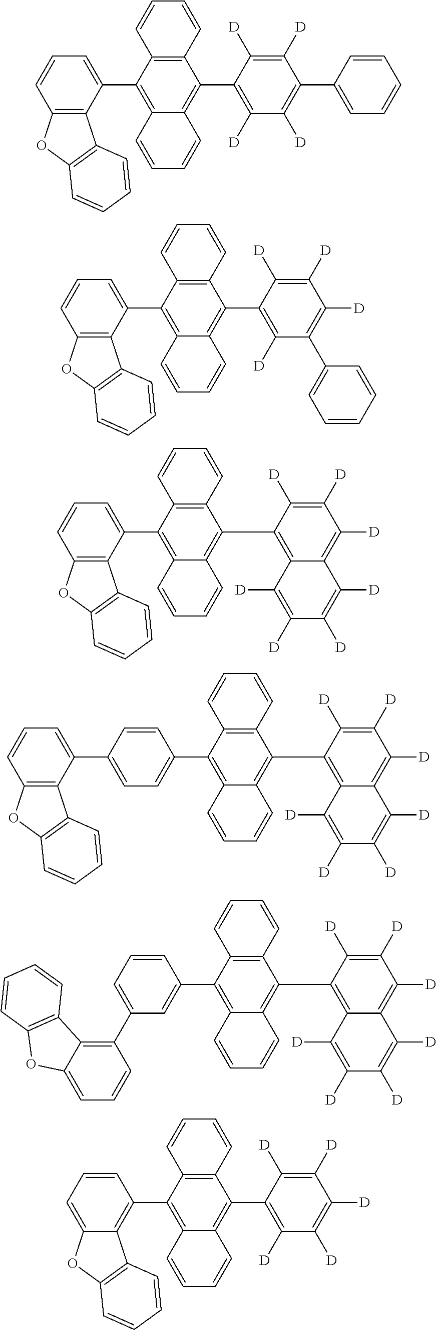

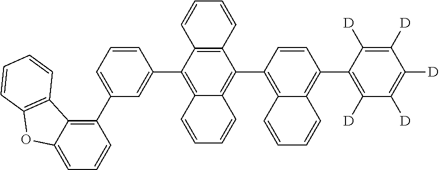 [SMILES: [2H]c1c([2H])c([2H])c(-c2ccc(-c3c4ccccc4c(-c4cccc(-c5cccc6oc7ccccc7c56)c4)c4ccccc34)c3ccccc23)c([2H])c1[2H]]

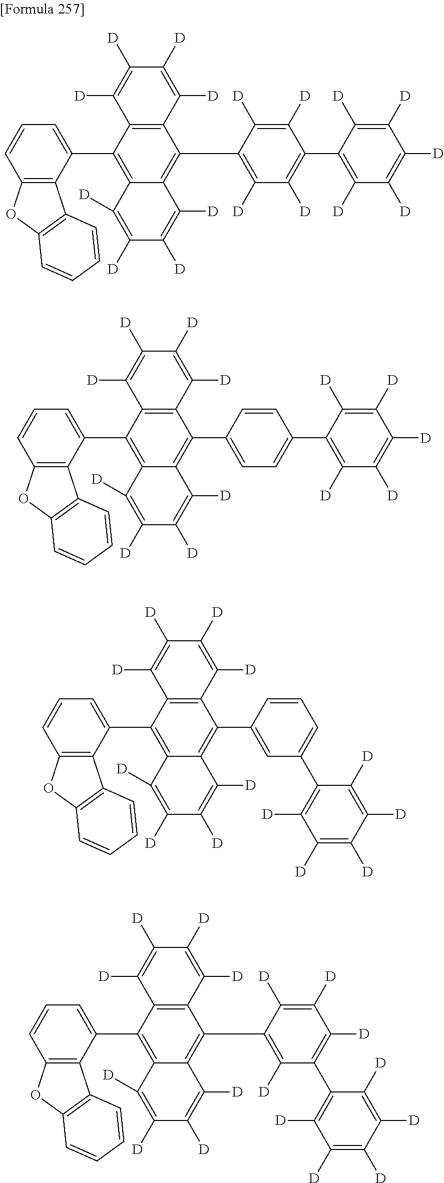

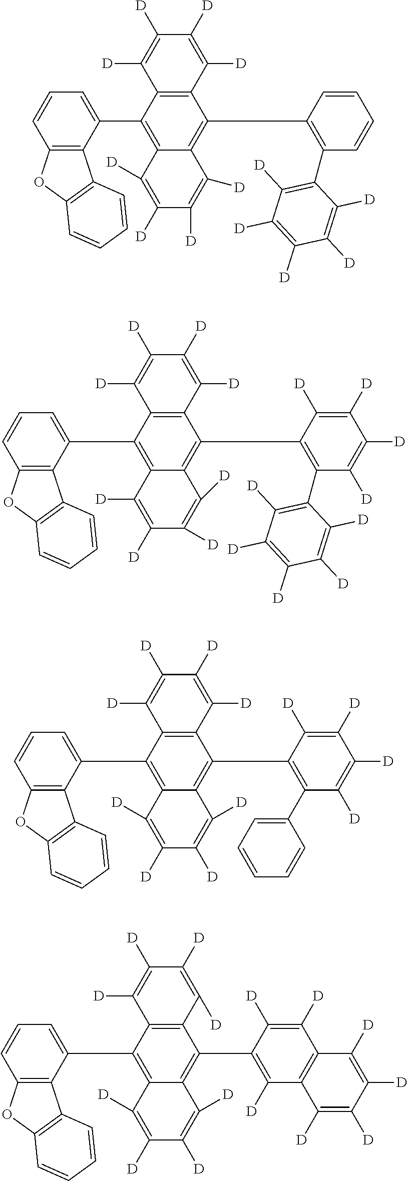

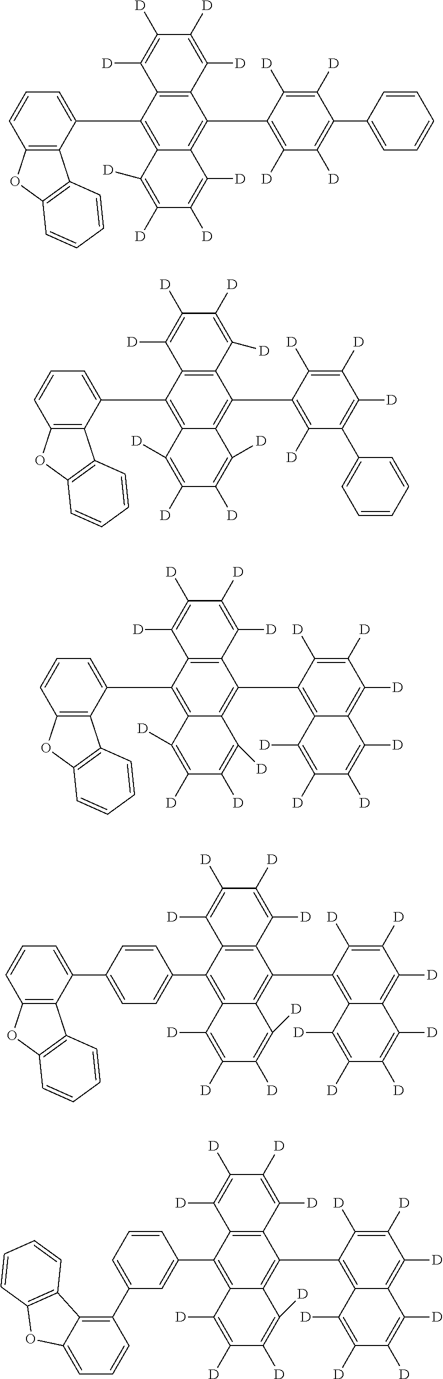

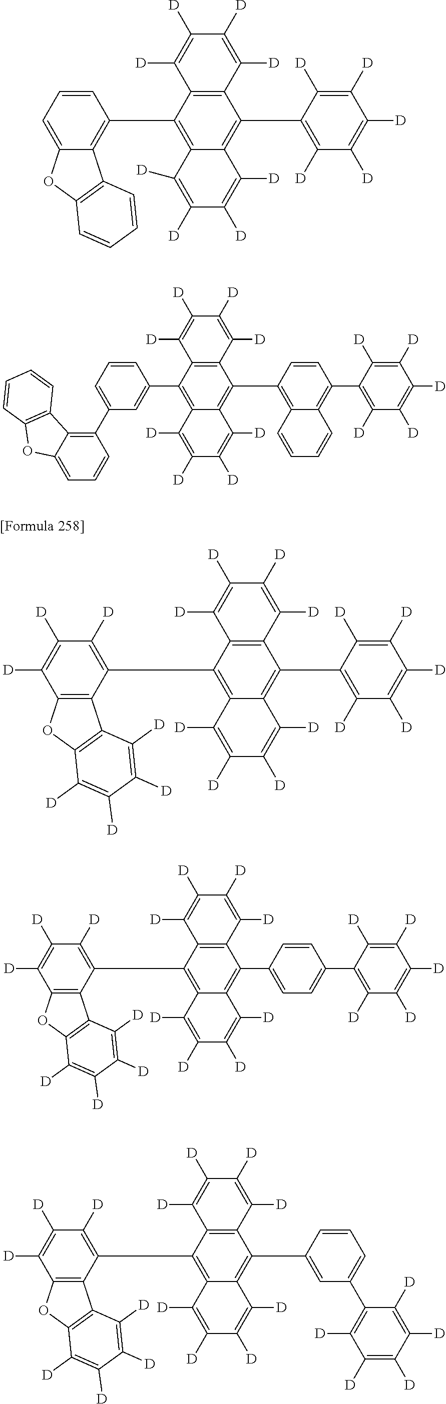

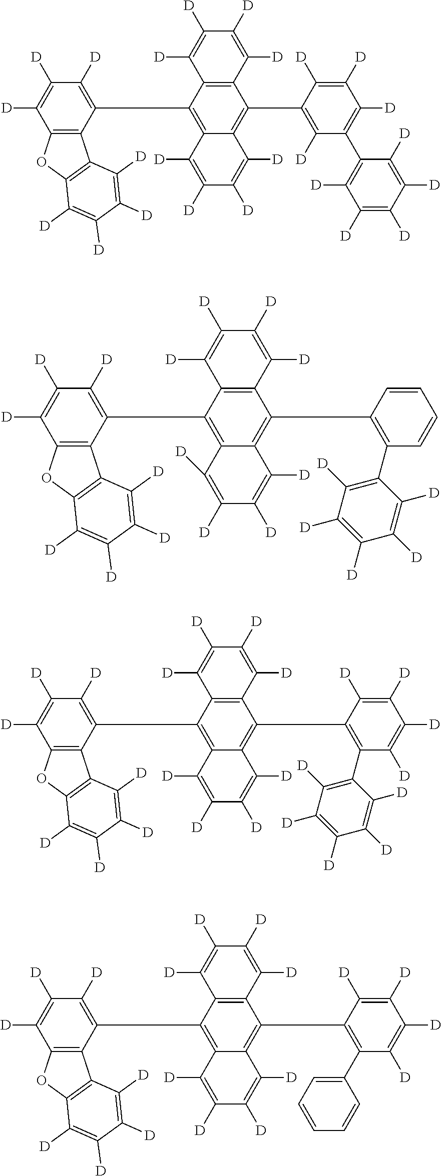

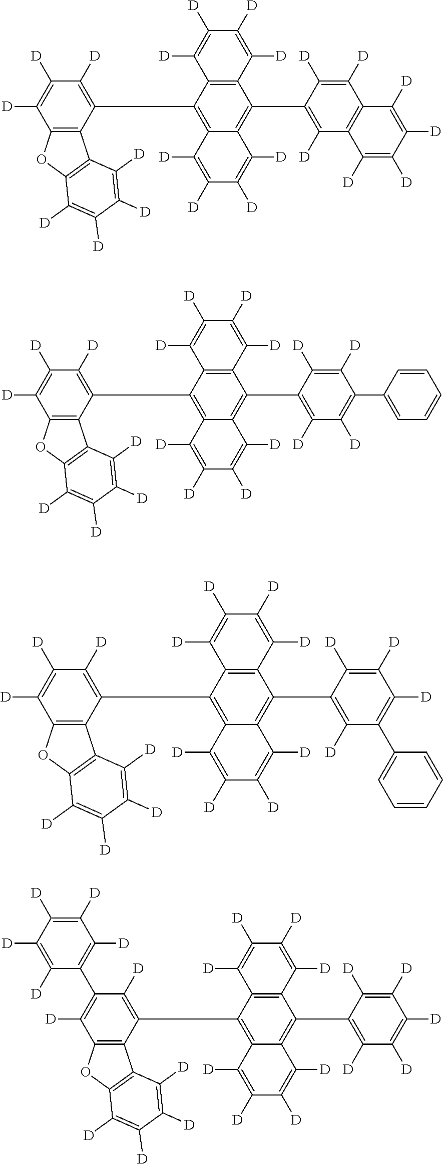

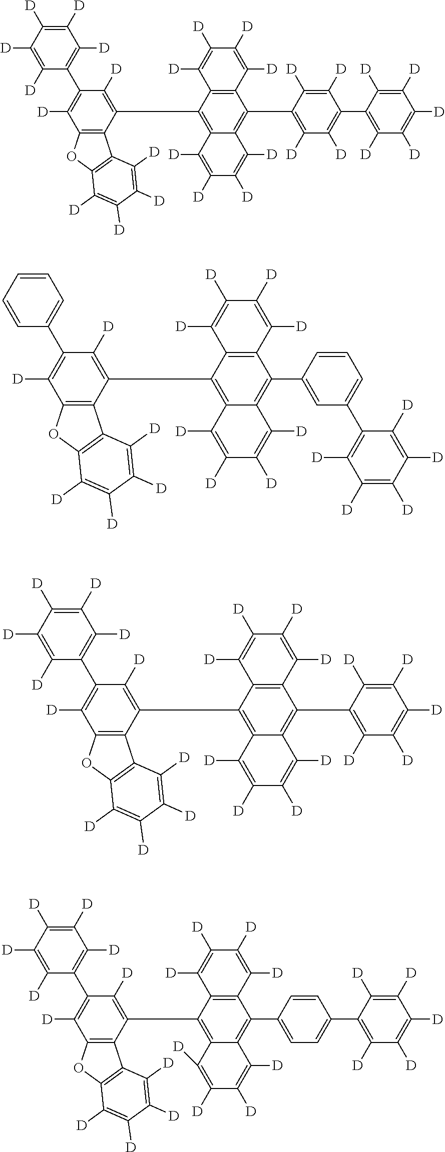

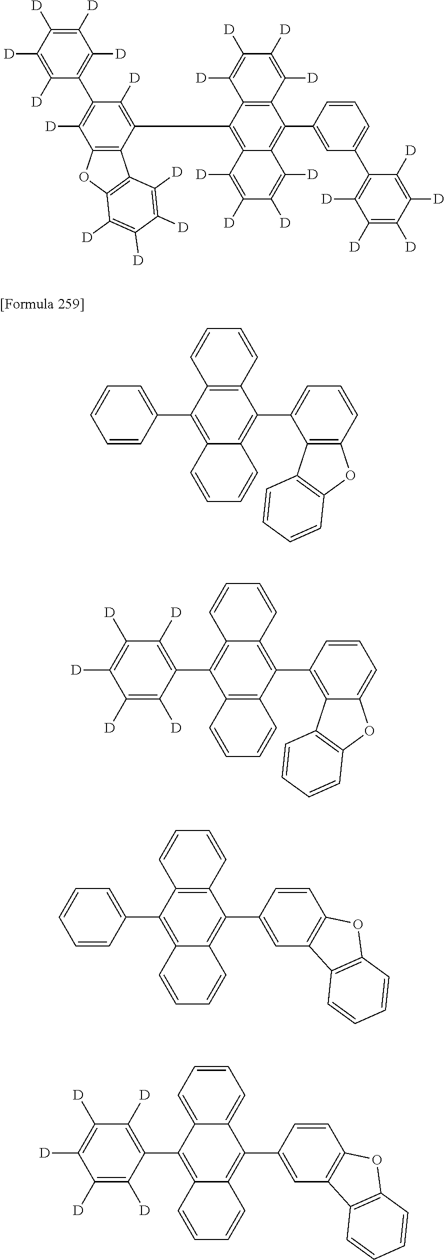 [SMILES: [2H]c1c([2H])c([2H])c(-c2cccc(-c3c4c([2H])c([2H])c([2H])c([2H])c4c(-c4c([2H])c(-c5c([2H])c([2H])c([2H])c([2H])c5[2H])c([2H])c5oc6c([2H])c([2H])c([2H])c([2H])c6c45)c4c([2H])c([2H])c([2H])c([2H])c34)c2)c([2H])c1[2H] ; c1ccc(-c2c3ccccc3c(-c3cccc4oc5ccccc5c34)c3ccccc23)cc1 ; [2H]c1c([2H])c([2H])c(-c2c3ccccc3c(-c3cccc4oc5ccccc5c34)c3ccccc23)c([2H])c1[2H] ; c1ccc(-c2c3ccccc3c(-c3ccc4oc5ccccc5c4c3)c3ccccc23)cc1 ; [2H]c1c([2H])c([2H])c(-c2c3ccccc3c(-c3ccc4oc5ccccc5c4c3)c3ccccc23)c([2H])c1[2H]]

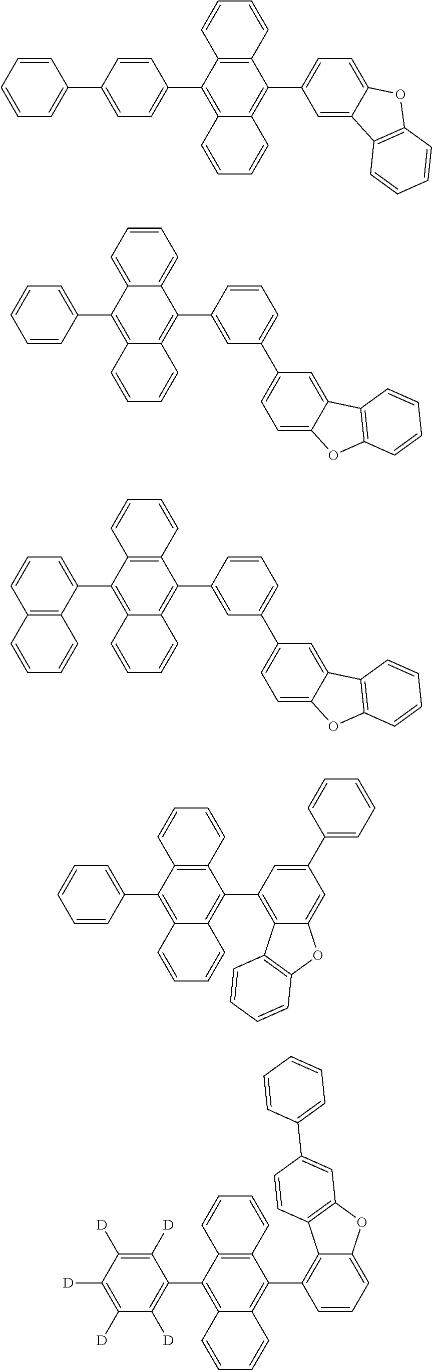 [SMILES: c1ccc(-c2ccc(-c3c4ccccc4c(-c4ccc5oc6ccccc6c5c4)c4ccccc34)cc2)cc1 ; c1ccc(-c2c3ccccc3c(-c3cccc(-c4ccc5oc6ccccc6c5c4)c3)c3ccccc23)cc1 ; c1cc(-c2ccc3oc4ccccc4c3c2)cc(-c2c3ccccc3c(-c3cccc4ccccc34)c3ccccc23)c1 ; c1ccc(-c2cc(-c3c4ccccc4c(-c4ccccc4)c4ccccc34)c3c(c2)oc2ccccc23)cc1 ; [2H]c1c([2H])c([2H])c(-c2c3ccccc3c(-c3cccc4oc5cc(-c6ccccc6)ccc5c34)c3ccccc23)c([2H])c1[2H]]

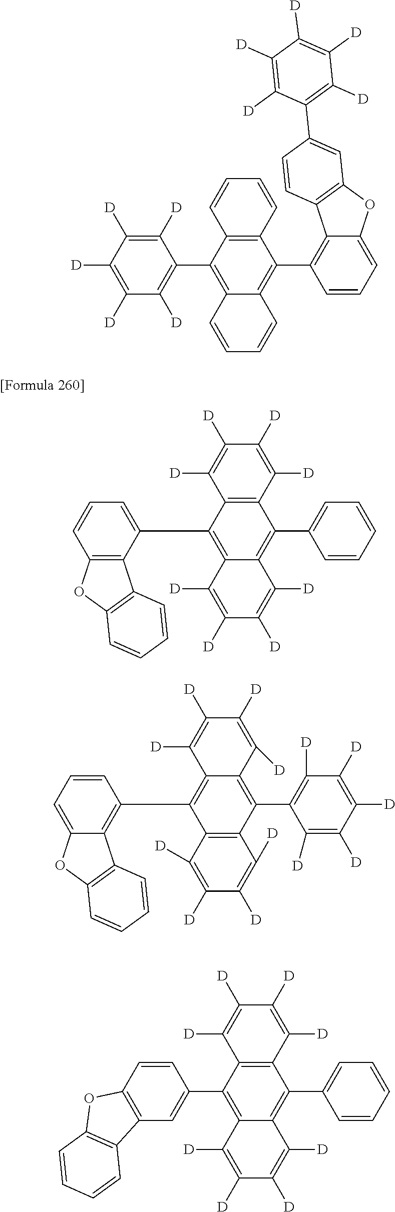

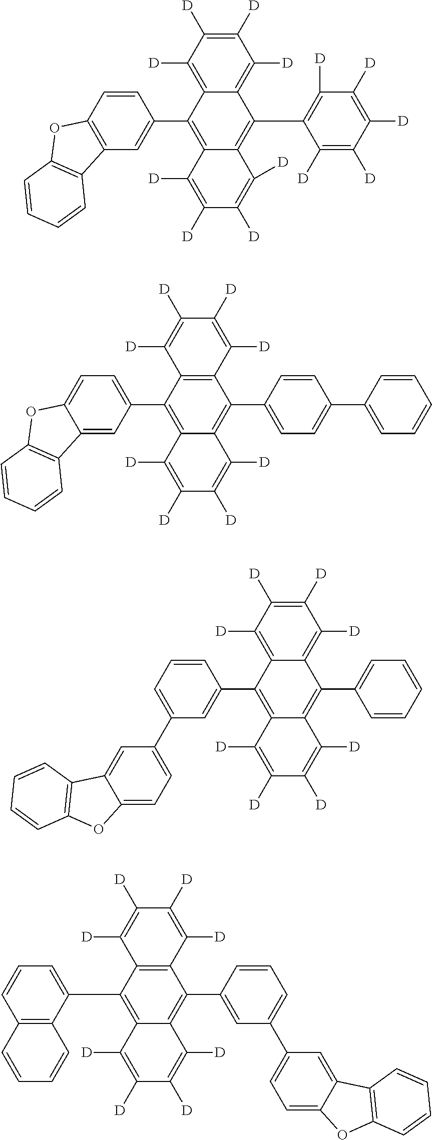 [SMILES: [2H]c1c([2H])c([2H])c(-c2c3c([2H])c([2H])c([2H])c([2H])c3c(-c3ccc4oc5ccccc5c4c3)c3c([2H])c([2H])c([2H])c([2H])c23)c([2H])c1[2H] ; [2H]c1c([2H])c([2H])c2c(-c3ccc4oc5ccccc5c4c3)c3c([2H])c([2H])c([2H])c([2H])c3c(-c3ccc(-c4ccccc4)cc3)c2c1[2H] ; [2H]c1c([2H])c([2H])c2c(-c3cccc(-c4ccc5oc6ccccc6c5c4)c3)c3c([2H])c([2H])c([2H])c([2H])c3c(-c3ccccc3)c2c1[2H] ; [2H]c1c([2H])c([2H])c2c(-c3cccc4ccccc34)c3c([2H])c([2H])c([2H])c([2H])c3c(-c3cccc(-c4ccc5oc6ccccc6c5c4)c3)c2c1[2H]]

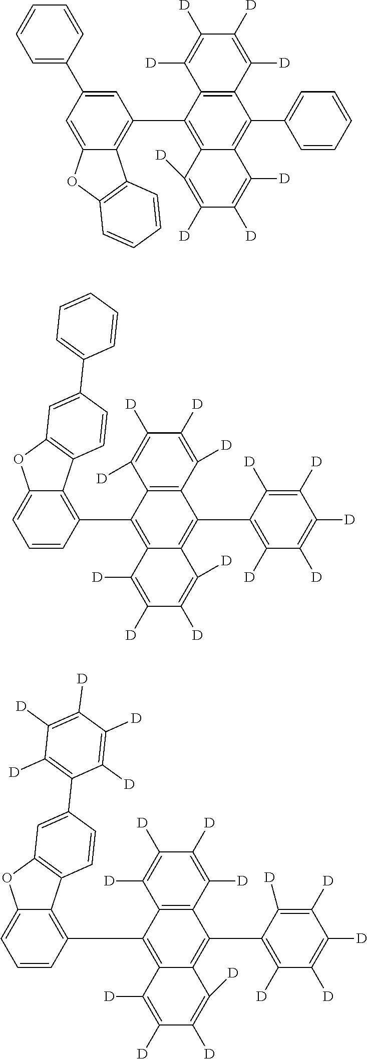 [SMILES: [2H]c1c([2H])c([2H])c2c(-c3cc(-c4ccccc4)cc4oc5ccccc5c34)c3c([2H])c([2H])c([2H])c([2H])c3c(-c3ccccc3)c2c1[2H] ; [2H]c1c([2H])c([2H])c(-c2c3c([2H])c([2H])c([2H])c([2H])c3c(-c3cccc4oc5cc(-c6ccccc6)ccc5c34)c3c([2H])c([2H])c([2H])c([2H])c23)c([2H])c1[2H] ; [2H]c1c([2H])c([2H])c(-c2ccc3c(c2)oc2cccc(-c4c5c([2H])c([2H])c([2H])c([2H])c5c(-c5c([2H])c([2H])c([2H])c([2H])c5[2H])c5c([2H])c([2H])c([2H])c([2H])c45)c23)c([2H])c1[2H]]

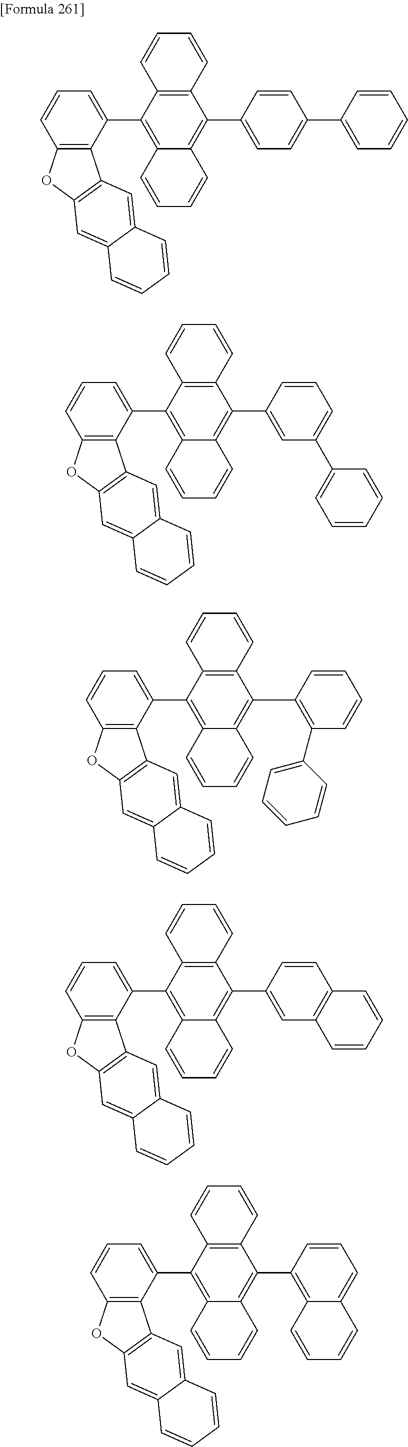

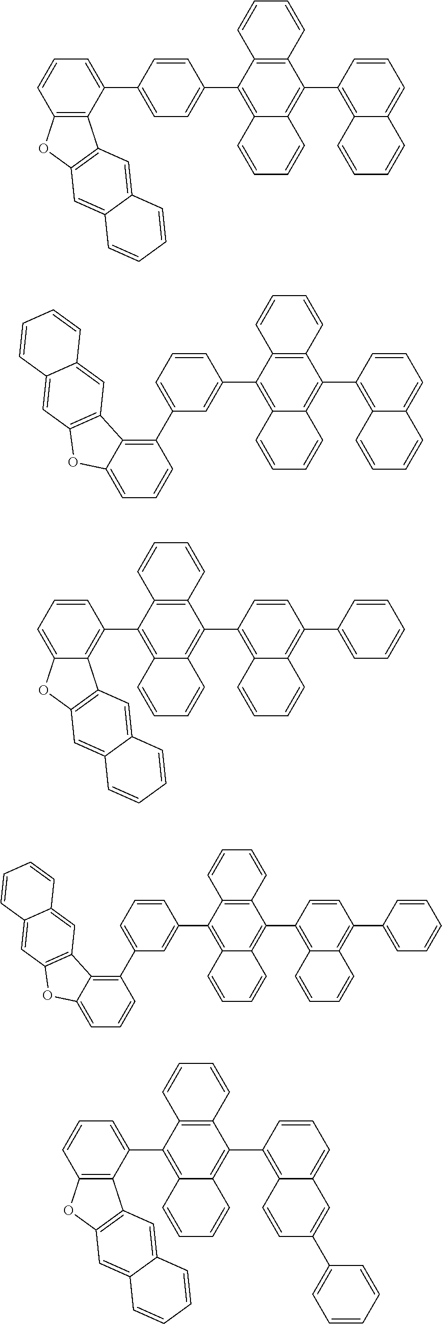

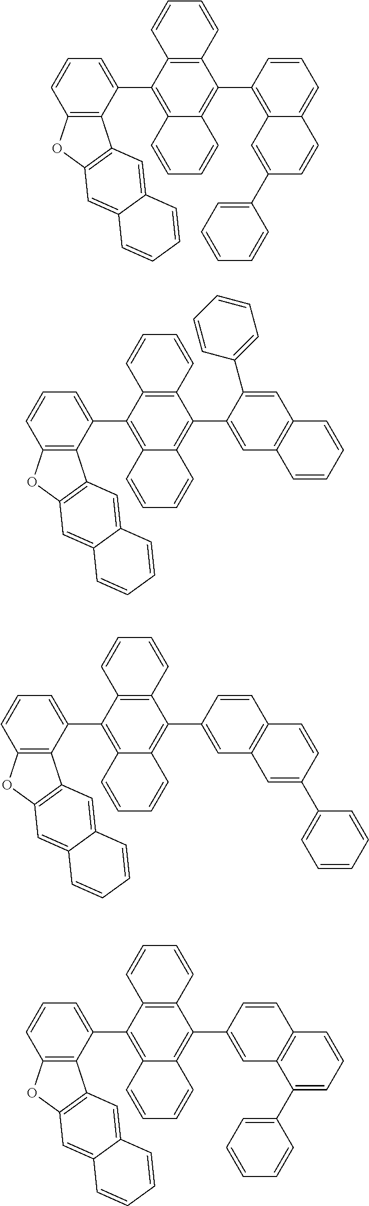

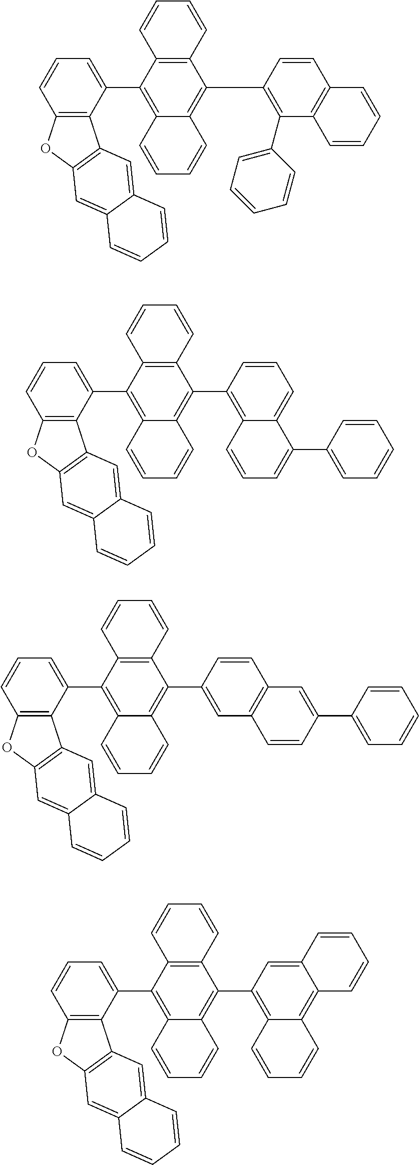

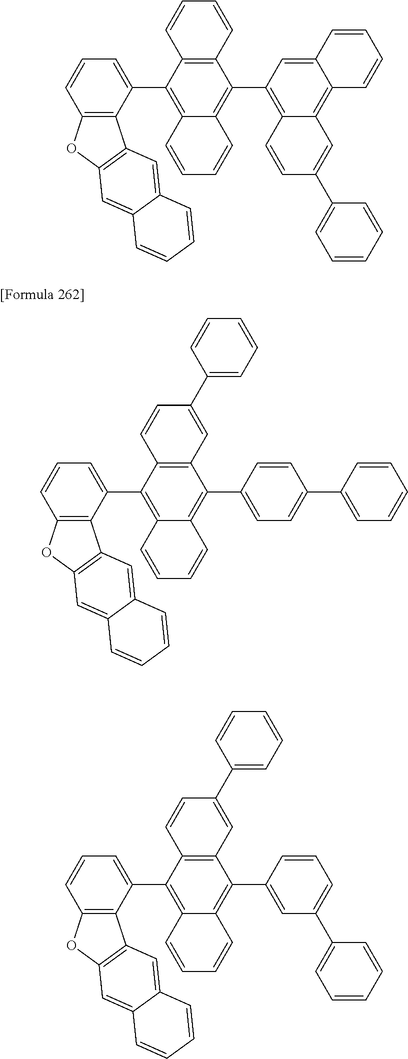 [SMILES: c1ccc(-c2ccc3c(-c4c5ccccc5c(-c5cccc6oc7cc8ccccc8cc7c56)c5ccccc45)cc4ccccc4c3c2)cc1 ; c1ccc(-c2ccc(-c3c4ccccc4c(-c4cccc5oc6cc7ccccc7cc6c45)c4ccc(-c5ccccc5)cc34)cc2)cc1 ; c1ccc(-c2cccc(-c3c4ccccc4c(-c4cccc5oc6cc7ccccc7cc6c45)c4ccc(-c5ccccc5)cc34)c2)cc1]

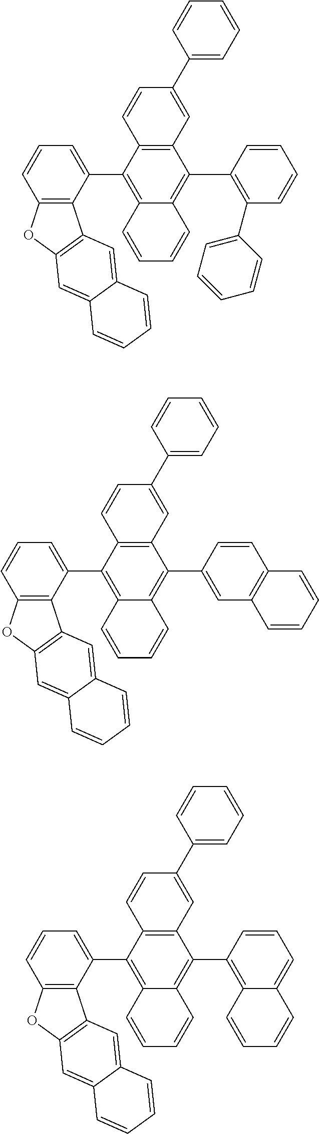 [SMILES: c1ccc(-c2ccc3c(-c4cccc5oc6cc7ccccc7cc6c45)c4ccccc4c(-c4ccccc4-c4ccccc4)c3c2)cc1 ; c1ccc(-c2ccc3c(-c4cccc5oc6cc7ccccc7cc6c45)c4ccccc4c(-c4ccc5ccccc5c4)c3c2)cc1 ; c1ccc(-c2ccc3c(-c4cccc5oc6cc7ccccc7cc6c45)c4ccccc4c(-c4cccc5ccccc45)c3c2)cc1]

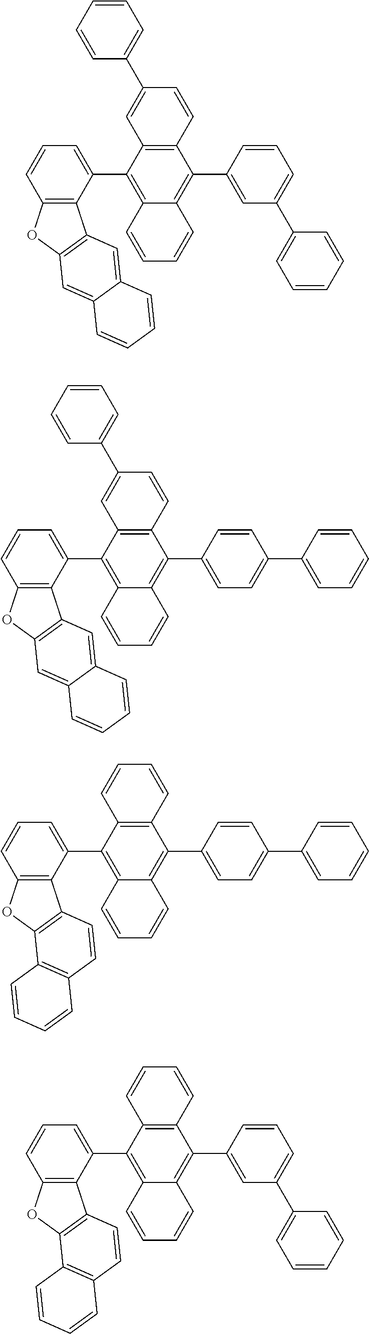

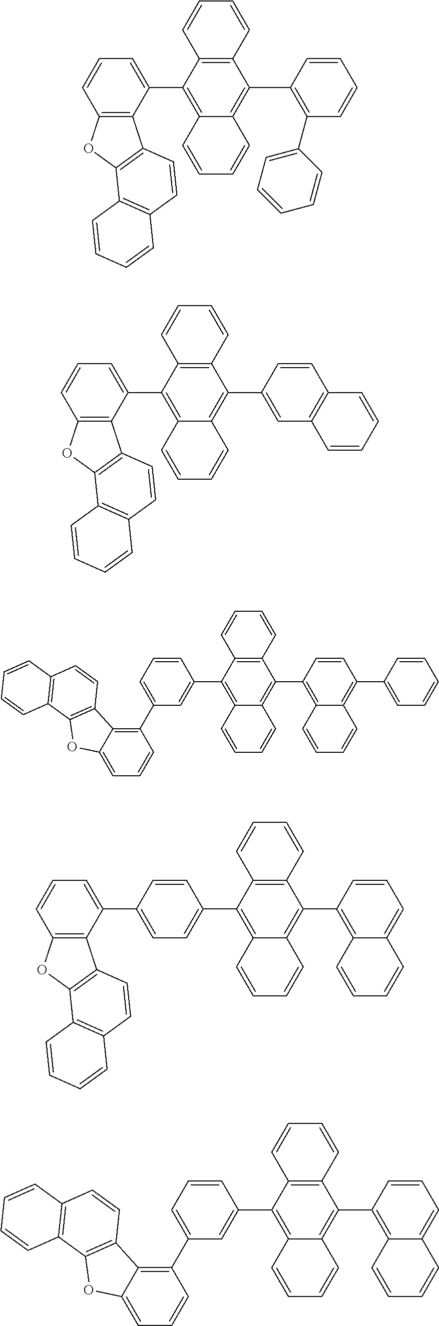 [SMILES: c1ccc(-c2ccccc2-c2c3ccccc3c(-c3cccc4oc5c6ccccc6ccc5c34)c3ccccc23)cc1 ; c1ccc2cc(-c3c4ccccc4c(-c4cccc5oc6c7ccccc7ccc6c45)c4ccccc34)ccc2c1 ; c1ccc(-c2ccc(-c3c4ccccc4c(-c4cccc(-c5cccc6oc7c8ccccc8ccc7c56)c4)c4ccccc34)c3ccccc23)cc1 ; c1ccc2c(-c3c4ccccc4c(-c4ccc(-c5cccc6oc7c8ccccc8ccc7c56)cc4)c4ccccc34)cccc2c1 ; c1cc(-c2c3ccccc3c(-c3cccc4ccccc34)c3ccccc23)cc(-c2cccc3oc4c5ccccc5ccc4c23)c1]

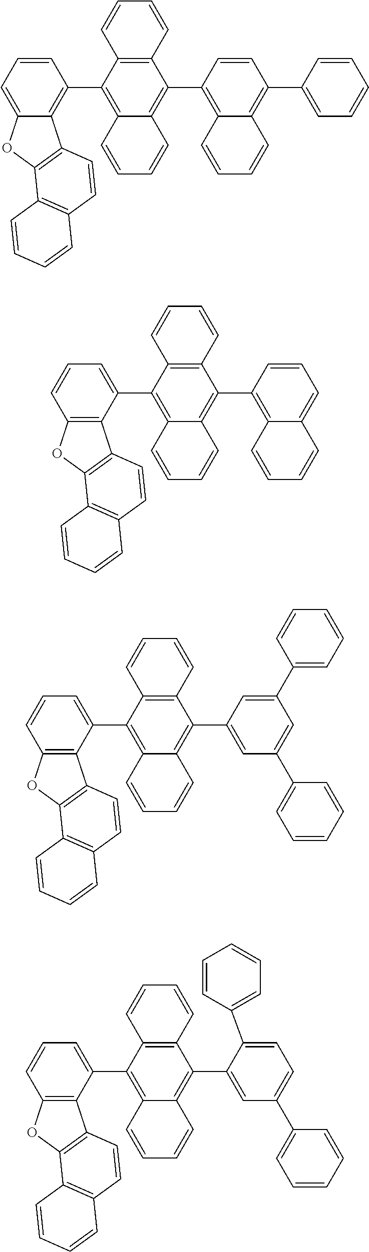 [SMILES: c1ccc(-c2ccc(-c3c4ccccc4c(-c4cccc5oc6c7ccccc7ccc6c45)c4ccccc34)c3ccccc23)cc1 ; c1ccc2c(-c3c4ccccc4c(-c4cccc5oc6c7ccccc7ccc6c45)c4ccccc34)cccc2c1 ; c1ccc(-c2cc(-c3ccccc3)cc(-c3c4ccccc4c(-c4cccc5oc6c7ccccc7ccc6c45)c4ccccc34)c2)cc1 ; c1ccc(-c2ccc(-c3ccccc3)c(-c3c4ccccc4c(-c4cccc5oc6c7ccccc7ccc6c45)c4ccccc34)c2)cc1]

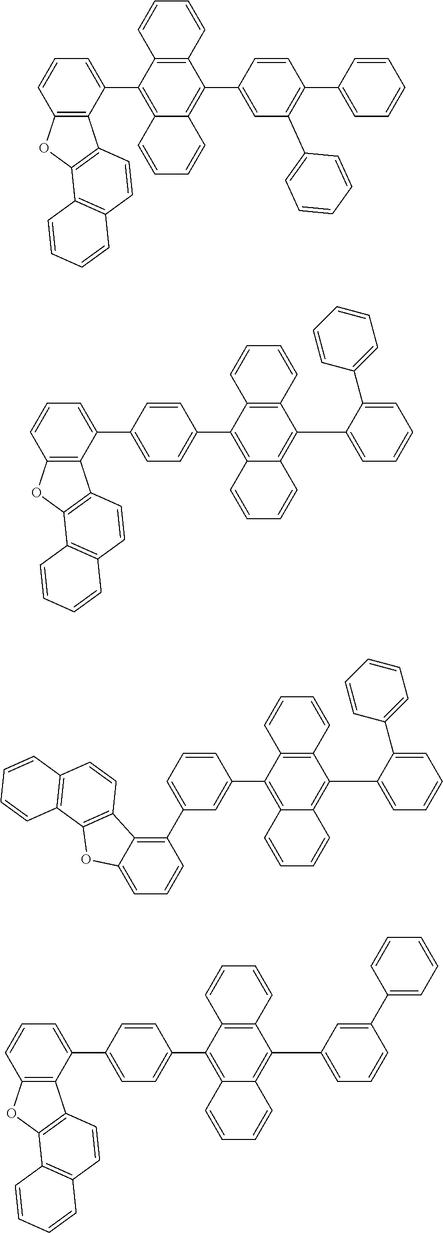 [SMILES: c1ccc(-c2ccc(-c3c4ccccc4c(-c4cccc5oc6c7ccccc7ccc6c45)c4ccccc34)cc2-c2ccccc2)cc1 ; c1ccc(-c2ccccc2-c2c3ccccc3c(-c3ccc(-c4cccc5oc6c7ccccc7ccc6c45)cc3)c3ccccc23)cc1 ; c1ccc(-c2ccccc2-c2c3ccccc3c(-c3cccc(-c4cccc5oc6c7ccccc7ccc6c45)c3)c3ccccc23)cc1 ; c1ccc(-c2cccc(-c3c4ccccc4c(-c4ccc(-c5cccc6oc7c8ccccc8ccc7c56)cc4)c4ccccc34)c2)cc1]

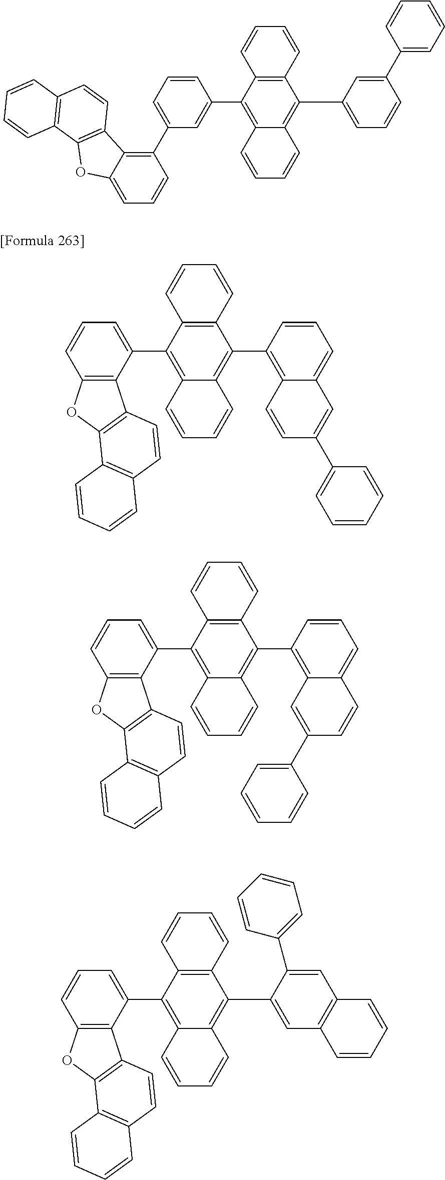 [SMILES: c1ccc(-c2cccc(-c3c4ccccc4c(-c4cccc(-c5cccc6oc7c8ccccc8ccc7c56)c4)c4ccccc34)c2)cc1 ; c1ccc(-c2ccc3c(-c4c5ccccc5c(-c5cccc6oc7c8ccccc8ccc7c56)c5ccccc45)cccc3c2)cc1 ; c1ccc(-c2ccc3cccc(-c4c5ccccc5c(-c5cccc6oc7c8ccccc8ccc7c56)c5ccccc45)c3c2)cc1 ; c1ccc(-c2cc3ccccc3cc2-c2c3ccccc3c(-c3cccc4oc5c6ccccc6ccc5c34)c3ccccc23)cc1]

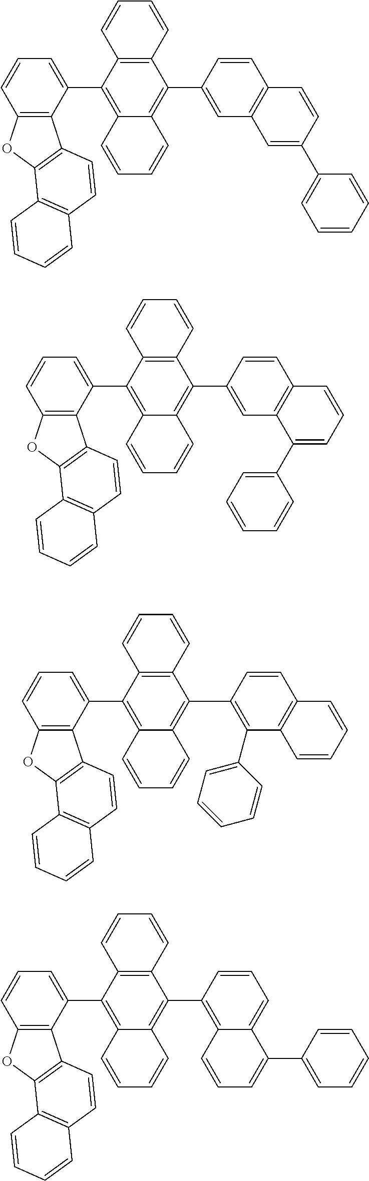 [SMILES: c1ccc(-c2ccc3ccc(-c4c5ccccc5c(-c5cccc6oc7c8ccccc8ccc7c56)c5ccccc45)cc3c2)cc1 ; c1ccc(-c2cccc3ccc(-c4c5ccccc5c(-c5cccc6oc7c8ccccc8ccc7c56)c5ccccc45)cc23)cc1 ; c1ccc(-c2c(-c3c4ccccc4c(-c4cccc5oc6c7ccccc7ccc6c45)c4ccccc34)ccc3ccccc23)cc1 ; c1ccc(-c2cccc3c(-c4c5ccccc5c(-c5cccc6oc7c8ccccc8ccc7c56)c5ccccc45)cccc23)cc1]

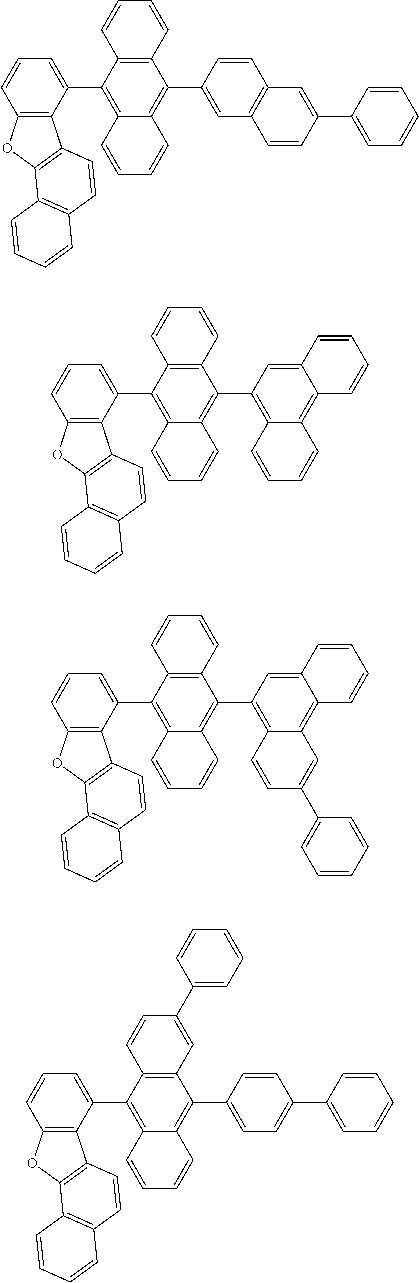 [SMILES: c1ccc(-c2ccc3cc(-c4c5ccccc5c(-c5cccc6oc7c8ccccc8ccc7c56)c5ccccc45)ccc3c2)cc1 ; c1ccc2c(c1)cc(-c1c3ccccc3c(-c3cccc4oc5c6ccccc6ccc5c34)c3ccccc13)c1ccccc12 ; c1ccc(-c2ccc3c(-c4c5ccccc5c(-c5cccc6oc7c8ccccc8ccc7c56)c5ccccc45)cc4ccccc4c3c2)cc1 ; c1ccc(-c2ccc(-c3c4ccccc4c(-c4cccc5oc6c7ccccc7ccc6c45)c4ccc(-c5ccccc5)cc34)cc2)cc1]

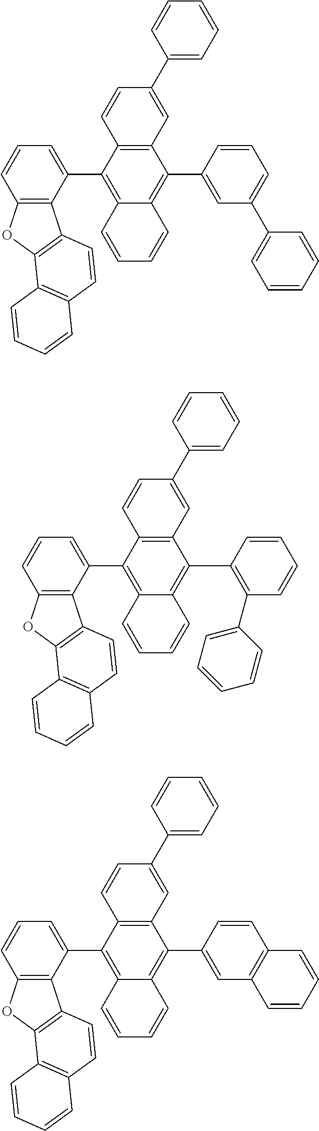 [SMILES: c1ccc(-c2cccc(-c3c4ccccc4c(-c4cccc5oc6c7ccccc7ccc6c45)c4ccc(-c5ccccc5)cc34)c2)cc1 ; c1ccc(-c2ccc3c(-c4cccc5oc6c7ccccc7ccc6c45)c4ccccc4c(-c4ccccc4-c4ccccc4)c3c2)cc1 ; c1ccc(-c2ccc3c(-c4cccc5oc6c7ccccc7ccc6c45)c4ccccc4c(-c4ccc5ccccc5c4)c3c2)cc1]

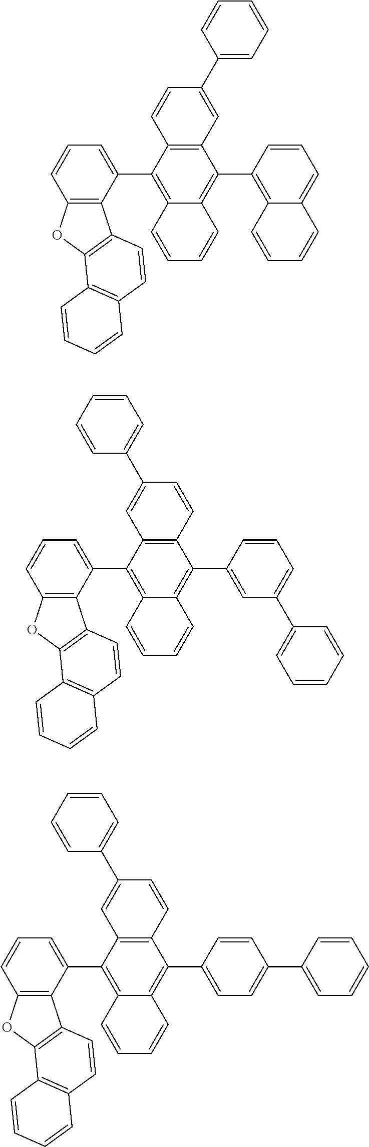

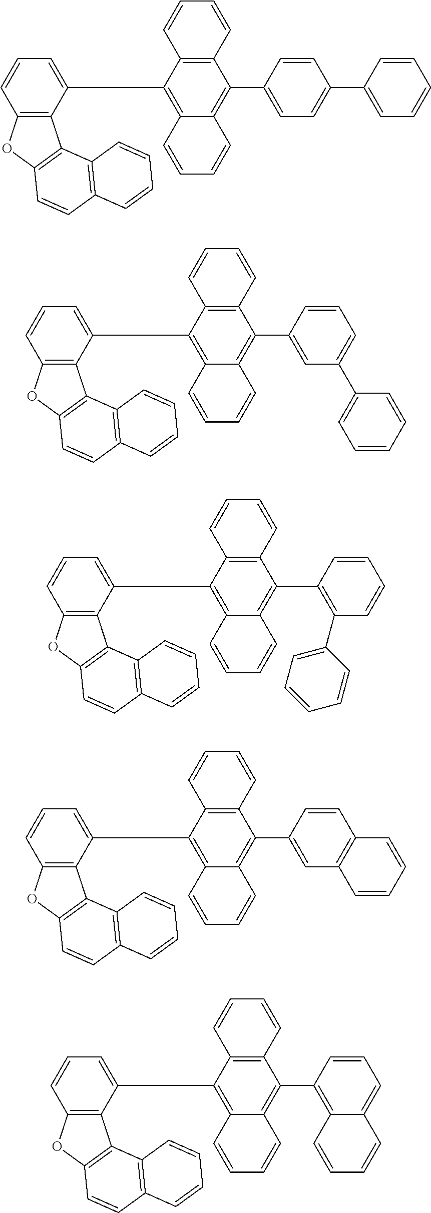 [SMILES: c1ccc(-c2ccc(-c3c4ccccc4c(-c4cccc5oc6ccc7ccccc7c6c45)c4ccccc34)cc2)cc1 ; c1ccc(-c2cccc(-c3c4ccccc4c(-c4cccc5oc6ccc7ccccc7c6c45)c4ccccc34)c2)cc1 ; c1ccc(-c2ccccc2-c2c3ccccc3c(-c3cccc4oc5ccc6ccccc6c5c34)c3ccccc23)cc1 ; c1ccc2cc(-c3c4ccccc4c(-c4cccc5oc6ccc7ccccc7c6c45)c4ccccc34)ccc2c1 ; c1ccc2c(-c3c4ccccc4c(-c4cccc5oc6ccc7ccccc7c6c45)c4ccccc34)cccc2c1]

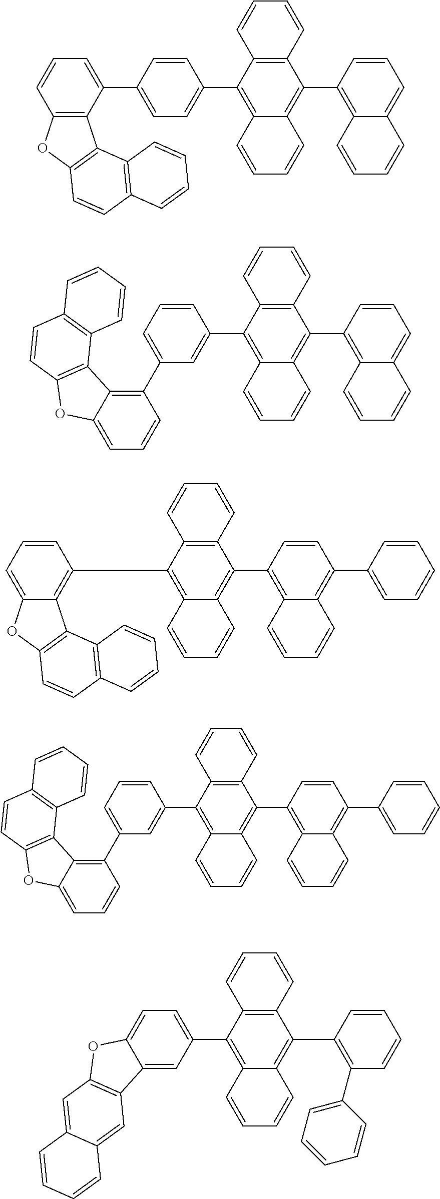 [SMILES: c1ccc2c(-c3c4ccccc4c(-c4ccc(-c5cccc6oc7ccc8ccccc8c7c56)cc4)c4ccccc34)cccc2c1 ; c1cc(-c2c3ccccc3c(-c3cccc4ccccc34)c3ccccc23)cc(-c2cccc3oc4ccc5ccccc5c4c23)c1 ; c1ccc(-c2ccc(-c3c4ccccc4c(-c4cccc5oc6ccc7ccccc7c6c45)c4ccccc34)c3ccccc23)cc1 ; c1ccc(-c2ccc(-c3c4ccccc4c(-c4cccc(-c5cccc6oc7ccc8ccccc8c7c56)c4)c4ccccc34)c3ccccc23)cc1 ; c1ccc(-c2ccccc2-c2c3ccccc3c(-c3ccc4oc5cc6ccccc6cc5c4c3)c3ccccc23)cc1]

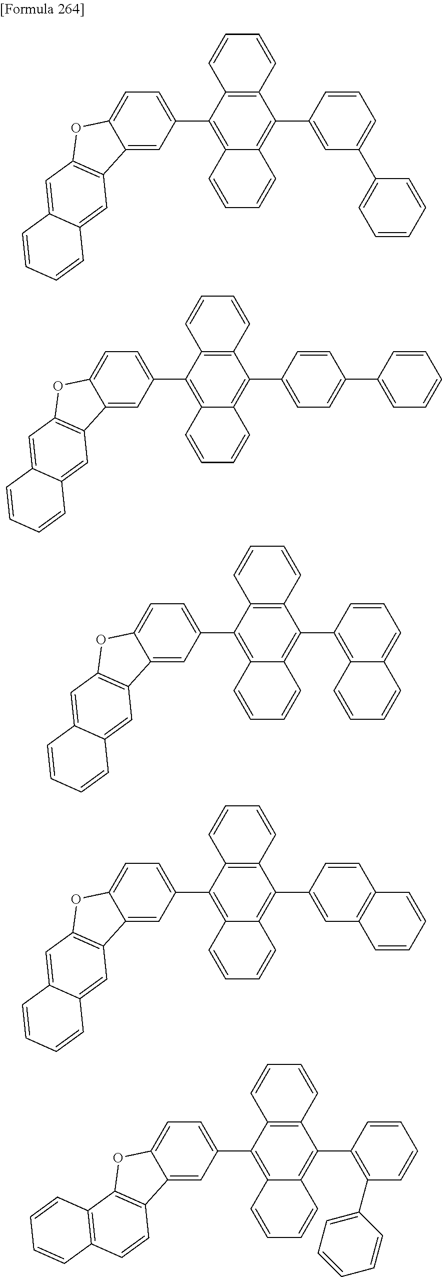 [SMILES: c1ccc(-c2cccc(-c3c4ccccc4c(-c4ccc5oc6cc7ccccc7cc6c5c4)c4ccccc34)c2)cc1 ; c1ccc(-c2ccc(-c3c4ccccc4c(-c4ccc5oc6cc7ccccc7cc6c5c4)c4ccccc34)cc2)cc1 ; c1ccc2cc3c(cc2c1)oc1ccc(-c2c4ccccc4c(-c4cccc5ccccc45)c4ccccc24)cc13 ; c1ccc2cc(-c3c4ccccc4c(-c4ccc5oc6cc7ccccc7cc6c5c4)c4ccccc34)ccc2c1 ; c1ccc(-c2ccccc2-c2c3ccccc3c(-c3ccc4oc5c6ccccc6ccc5c4c3)c3ccccc23)cc1]

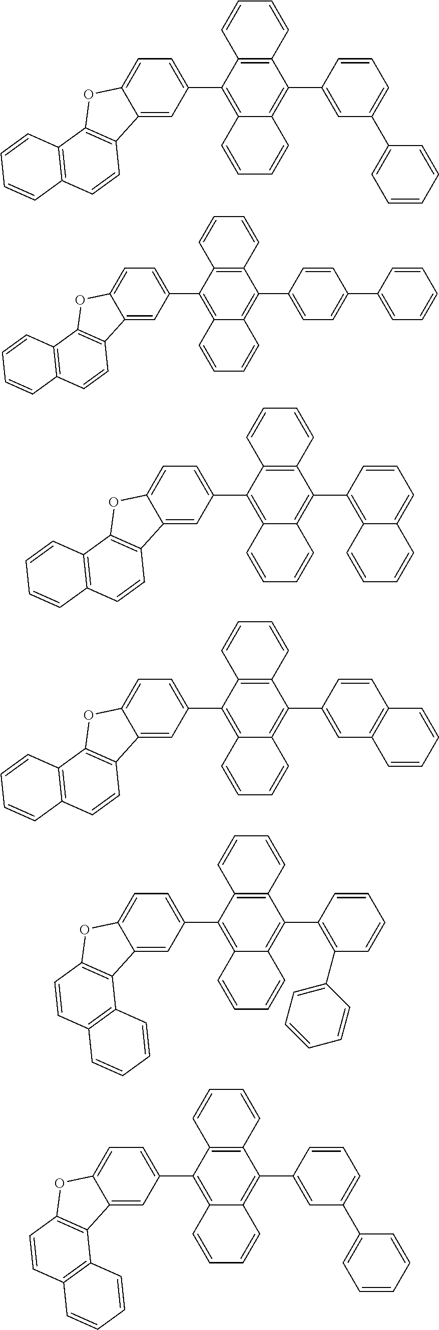

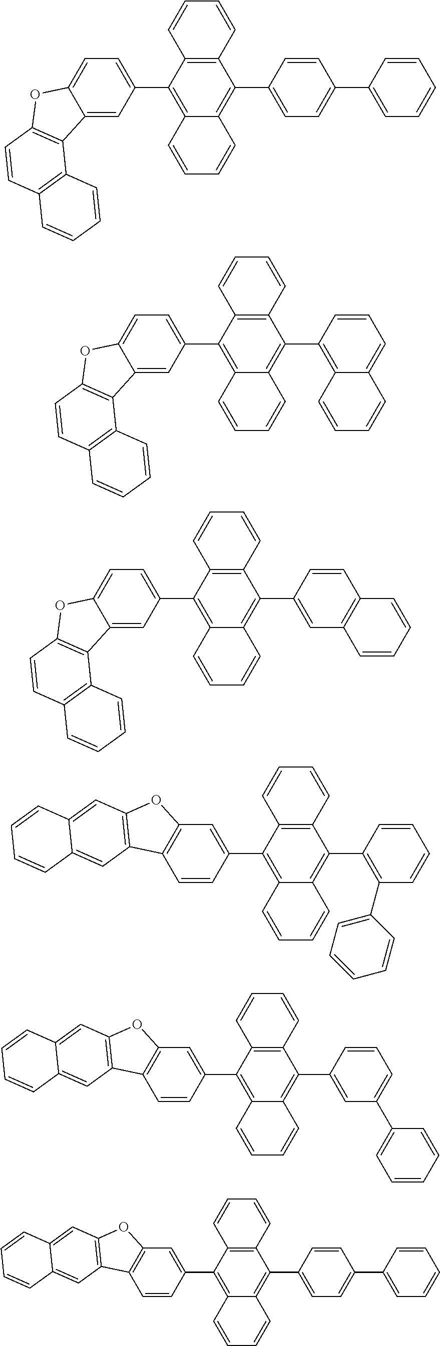

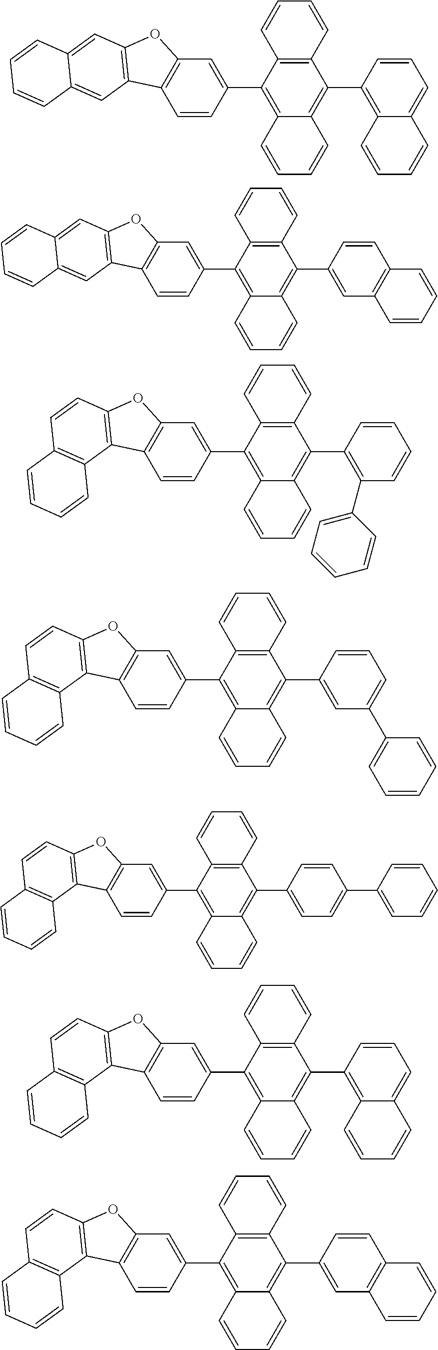

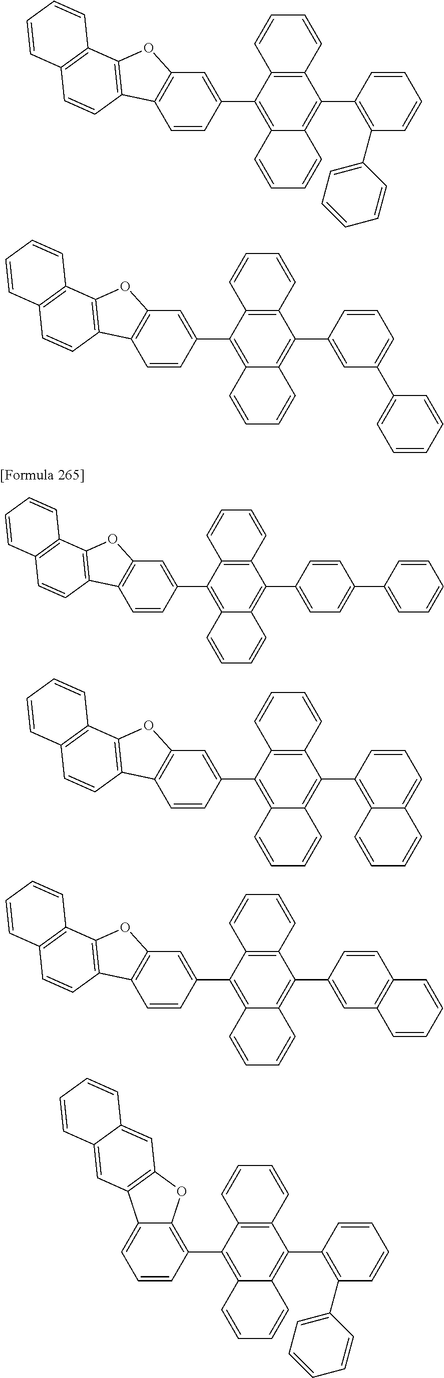

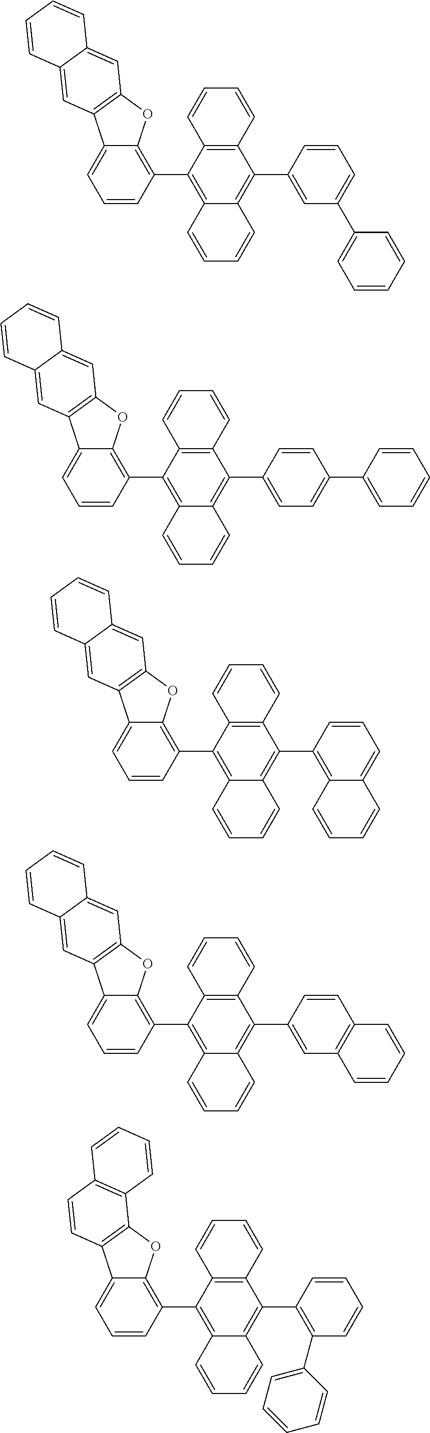 [SMILES: c1ccc(-c2cccc(-c3c4ccccc4c(-c4cccc5c4oc4cc6ccccc6cc45)c4ccccc34)c2)cc1 ; c1ccc(-c2ccc(-c3c4ccccc4c(-c4cccc5c4oc4cc6ccccc6cc45)c4ccccc34)cc2)cc1 ; c1ccc2cc3c(cc2c1)oc1c(-c2c4ccccc4c(-c4cccc5ccccc45)c4ccccc24)cccc13 ; c1ccc2cc(-c3c4ccccc4c(-c4cccc5c4oc4cc6ccccc6cc45)c4ccccc34)ccc2c1 ; c1ccc(-c2ccccc2-c2c3ccccc3c(-c3cccc4c3oc3c5ccccc5ccc43)c3ccccc23)cc1]

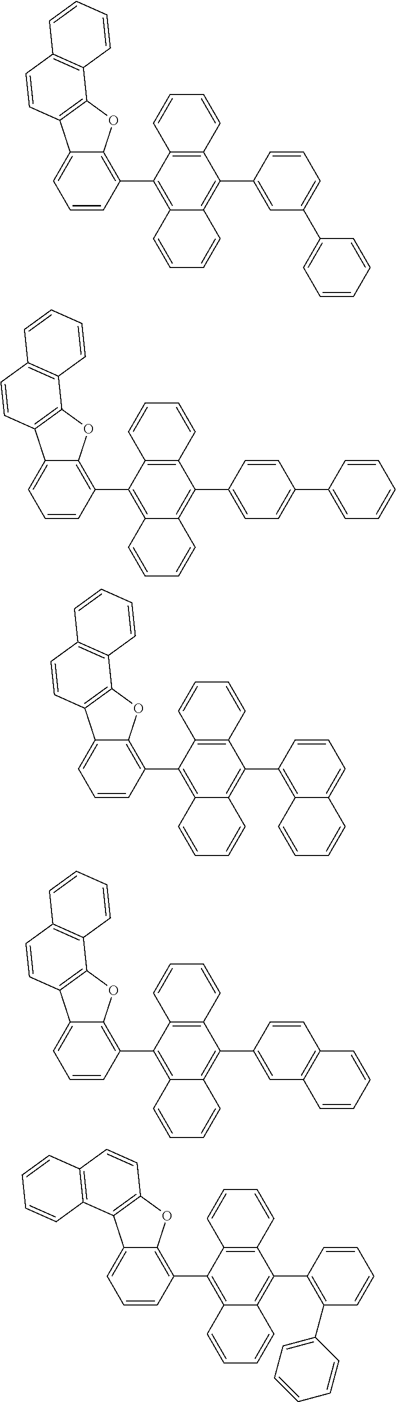

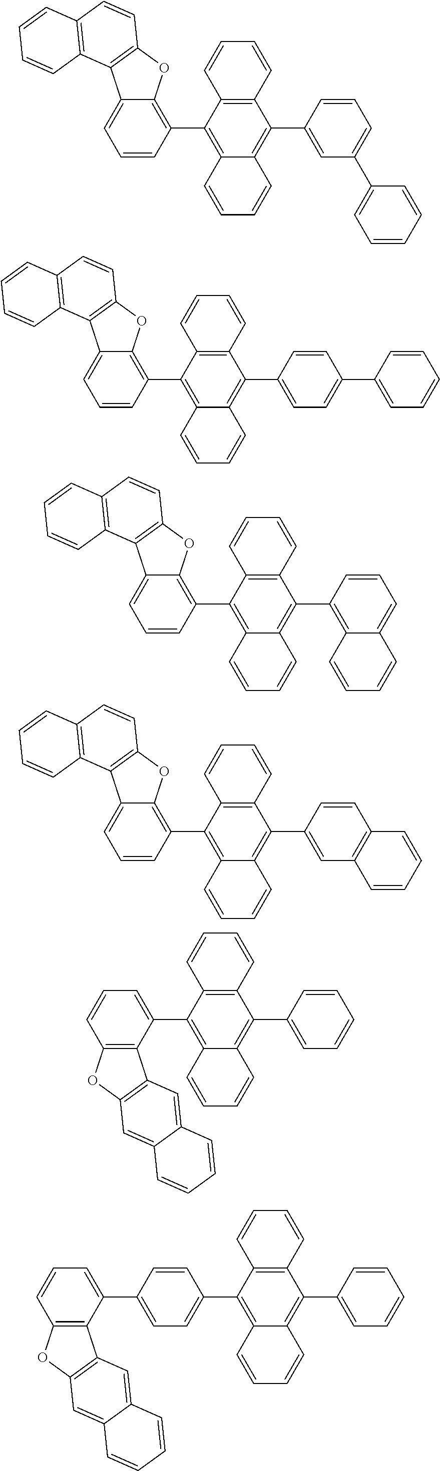

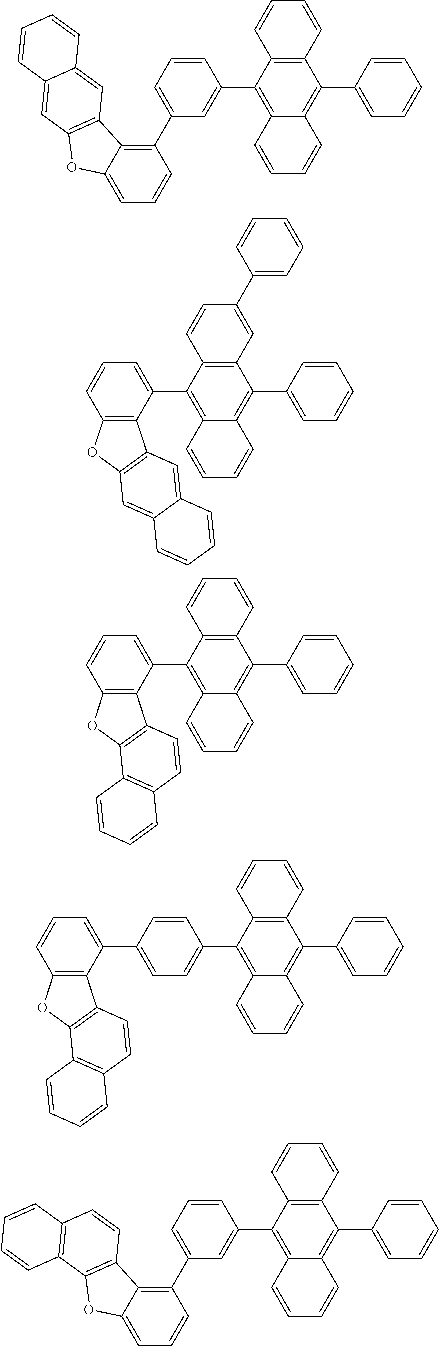

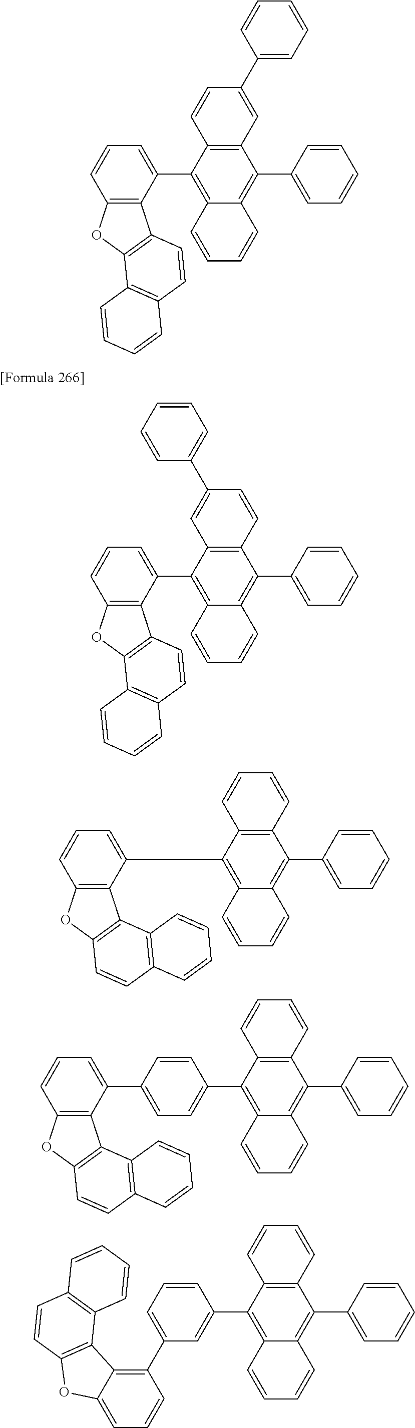

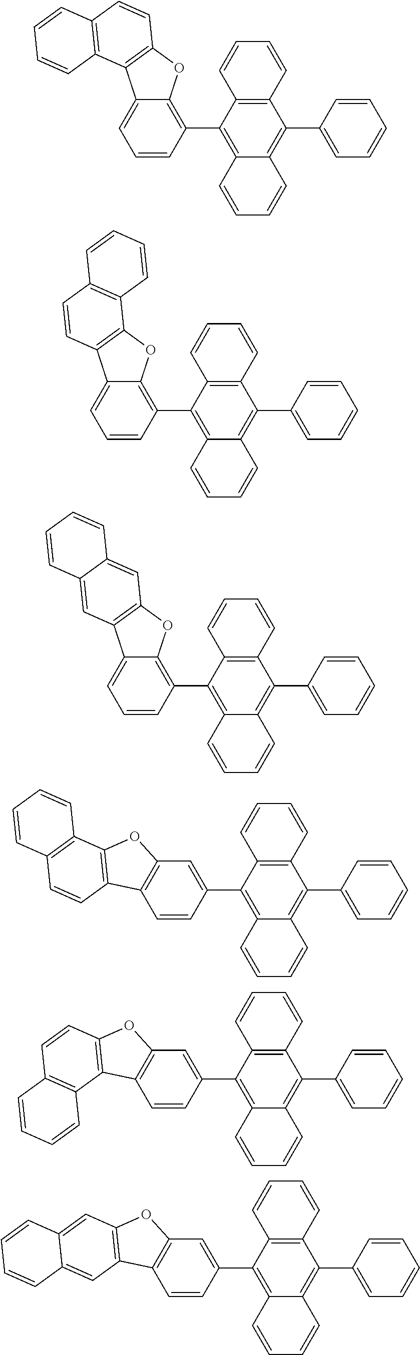

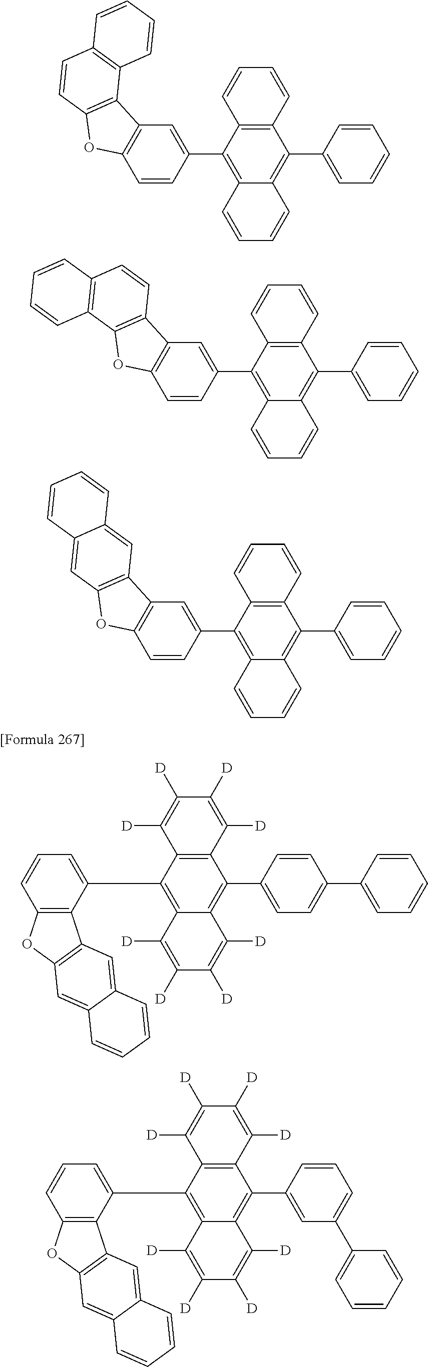

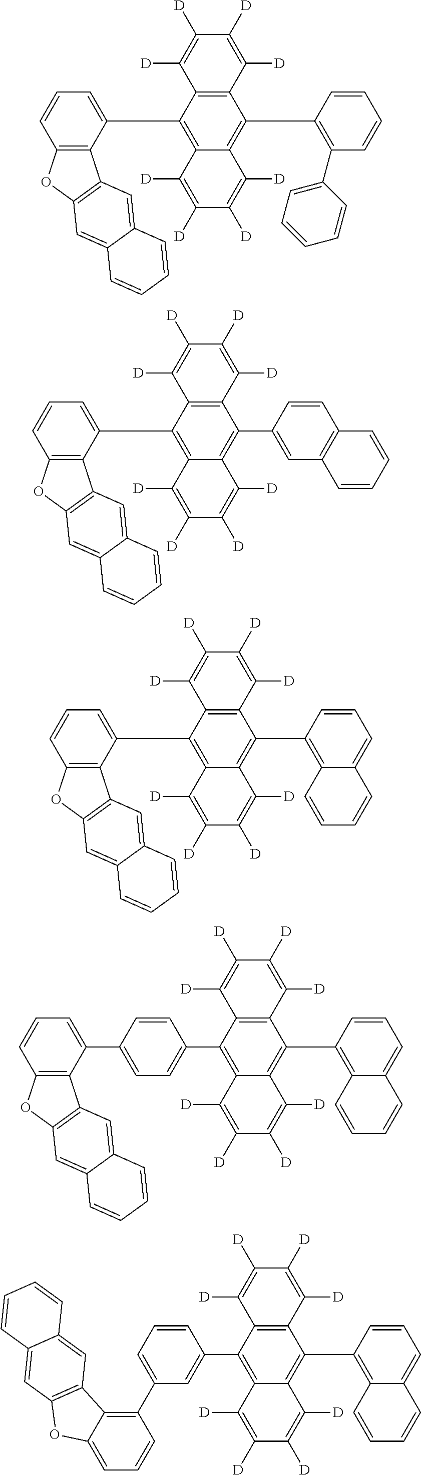

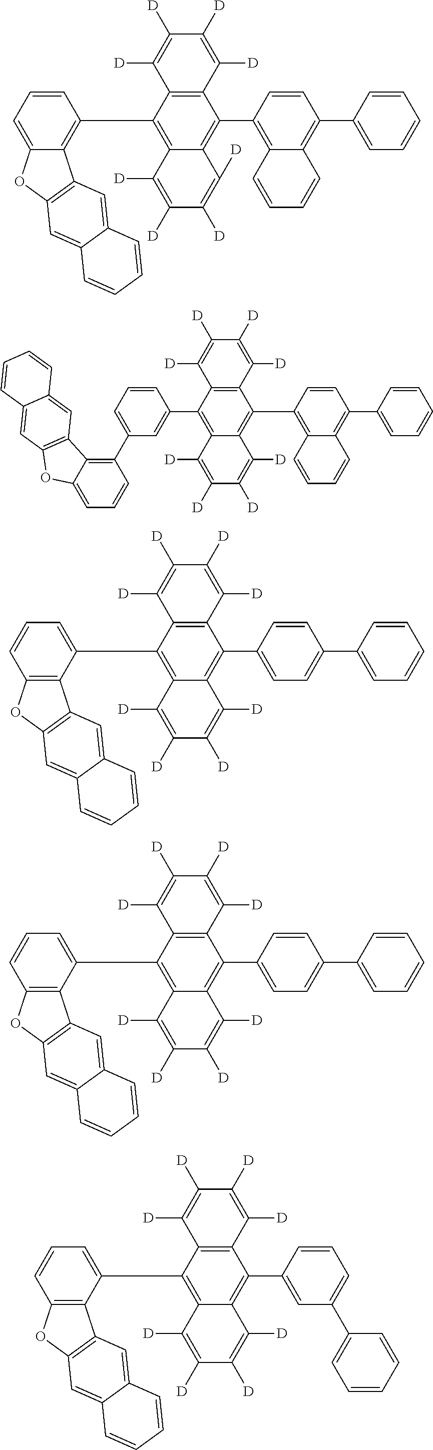

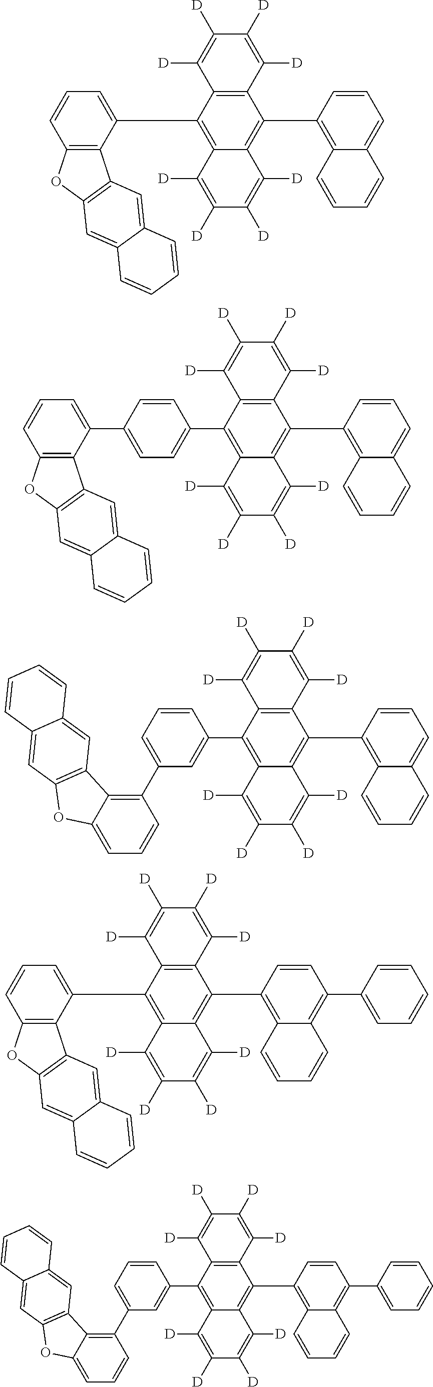

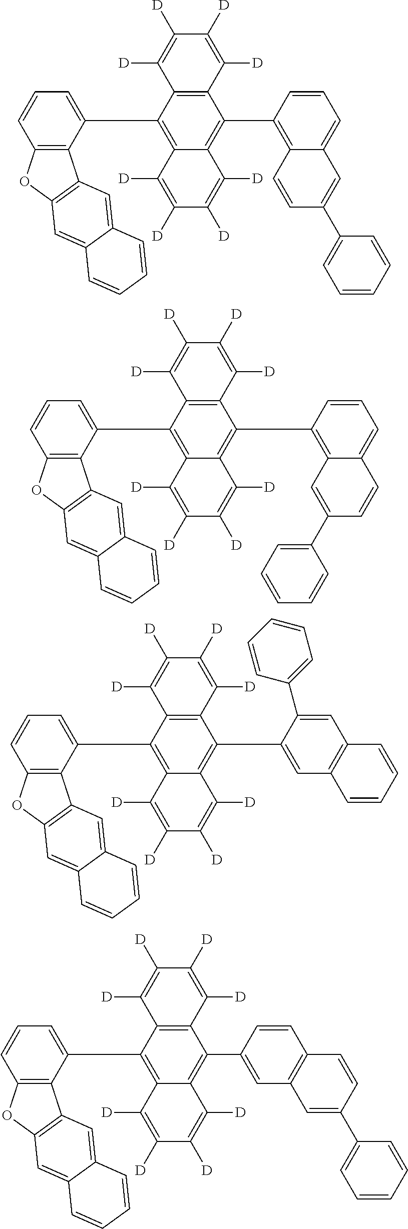

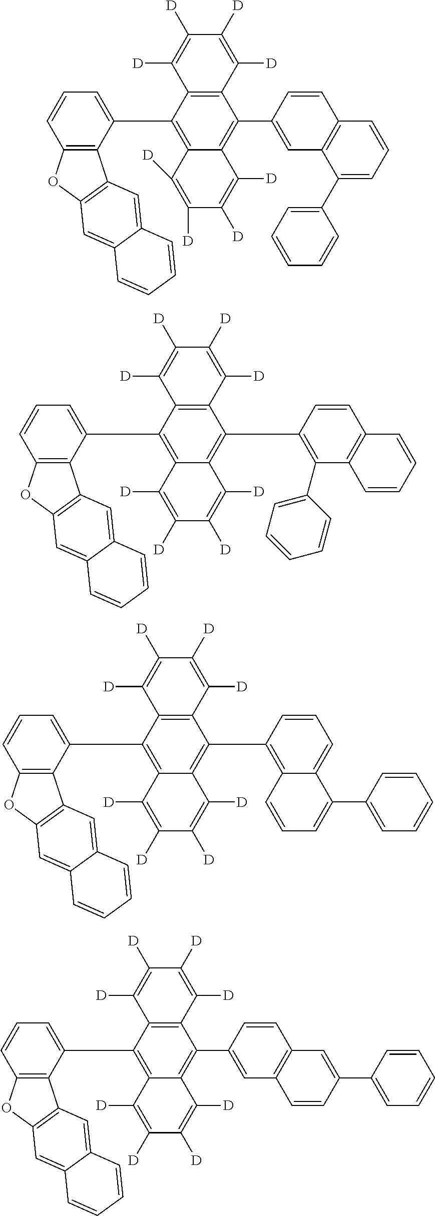

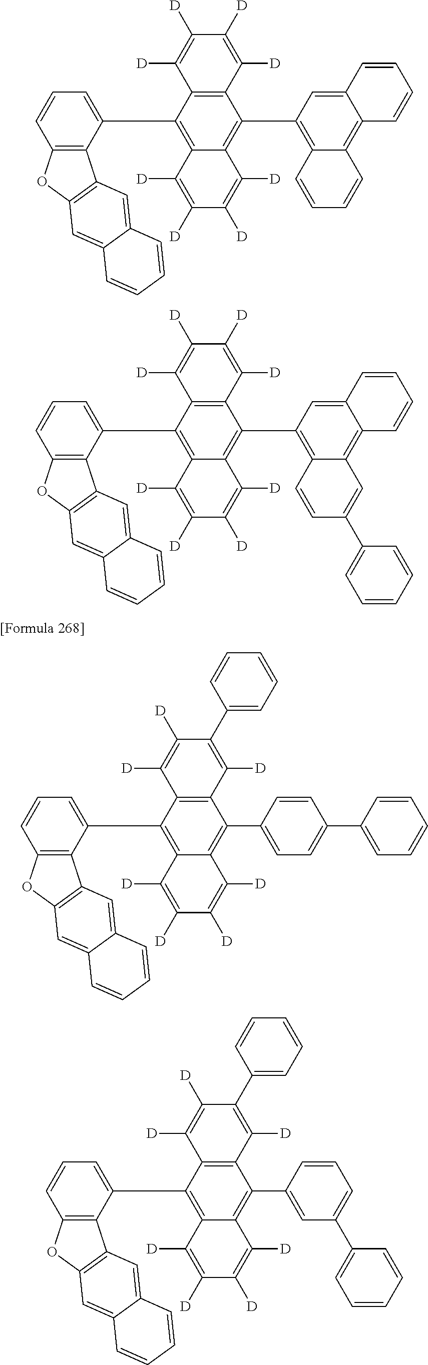

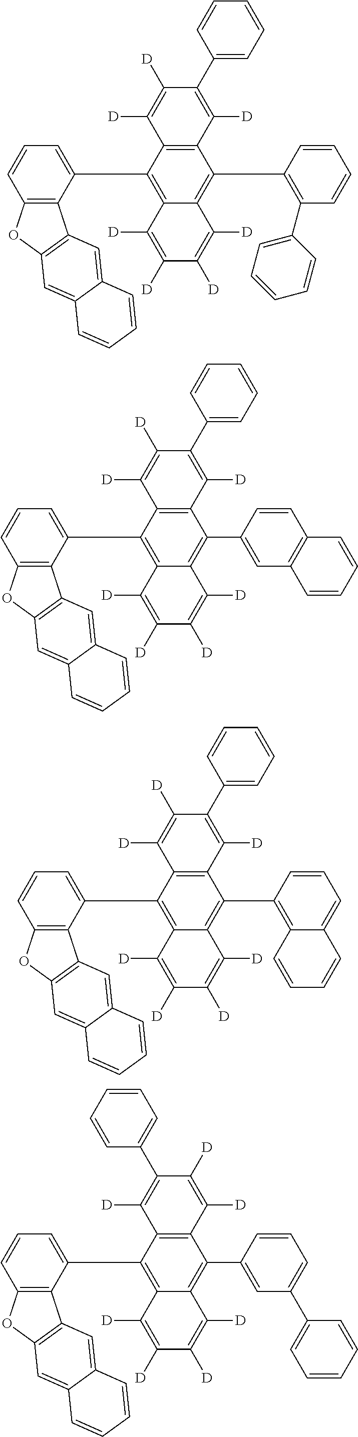

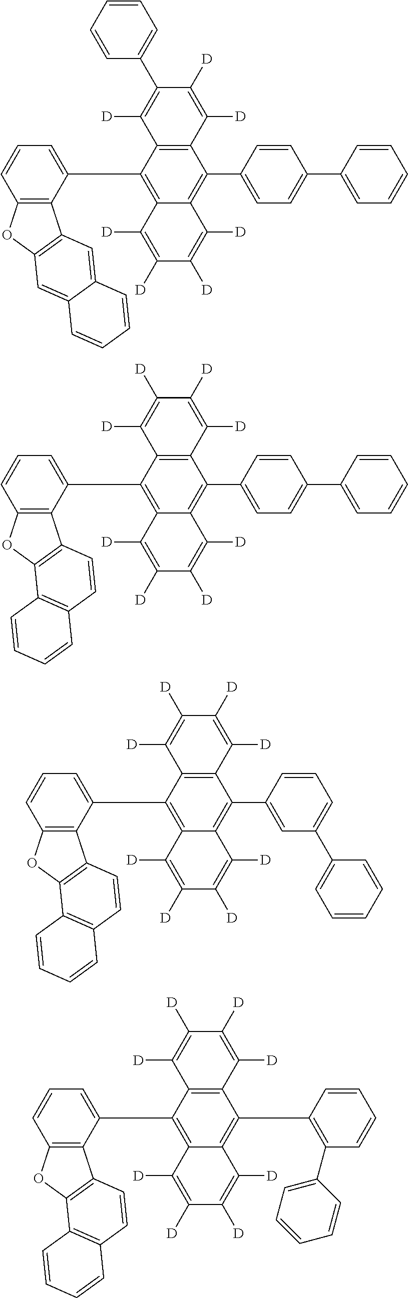

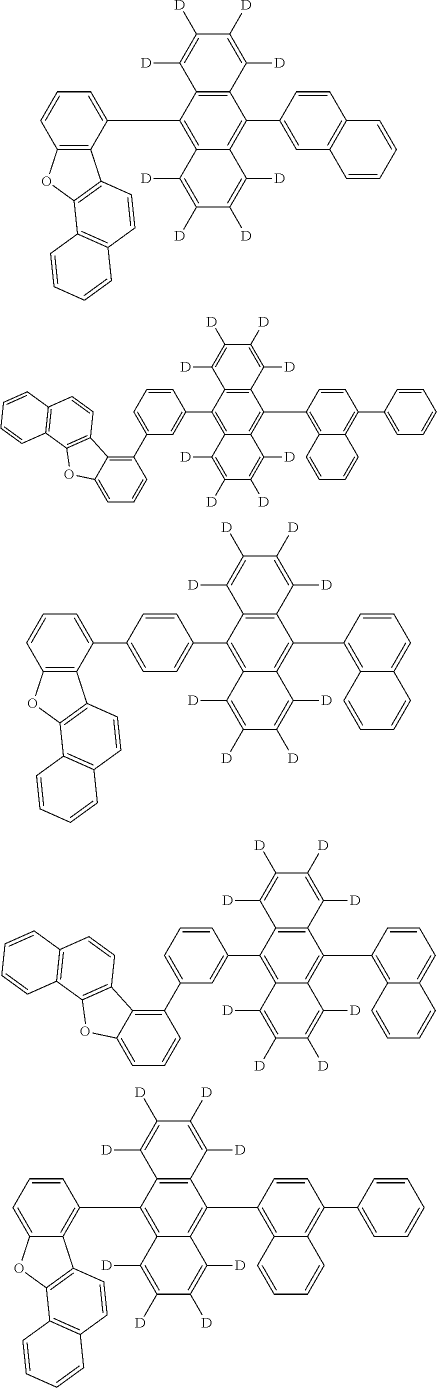

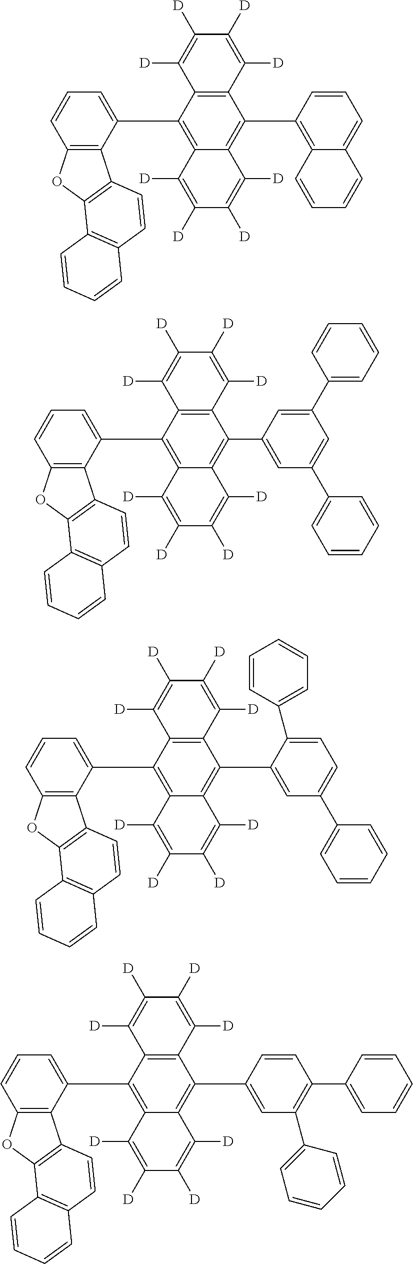 [SMILES: [2H]c1c([2H])c([2H])c2c(-c3cccc4oc5c6ccccc6ccc5c34)c3c([2H])c([2H])c([2H])c([2H])c3c(-c3cccc4ccccc34)c2c1[2H] ; [2H]c1c([2H])c([2H])c2c(-c3cccc4oc5c6ccccc6ccc5c34)c3c([2H])c([2H])c([2H])c([2H])c3c(-c3cc(-c4ccccc4)cc(-c4ccccc4)c3)c2c1[2H] ; [2H]c1c([2H])c([2H])c2c(-c3cccc4oc5c6ccccc6ccc5c34)c3c([2H])c([2H])c([2H])c([2H])c3c(-c3cc(-c4ccccc4)ccc3-c3ccccc3)c2c1[2H] ; [2H]c1c([2H])c([2H])c2c(-c3cccc4oc5c6ccccc6ccc5c34)c3c([2H])c([2H])c([2H])c([2H])c3c(-c3ccc(-c4ccccc4)c(-c4ccccc4)c3)c2c1[2H]]

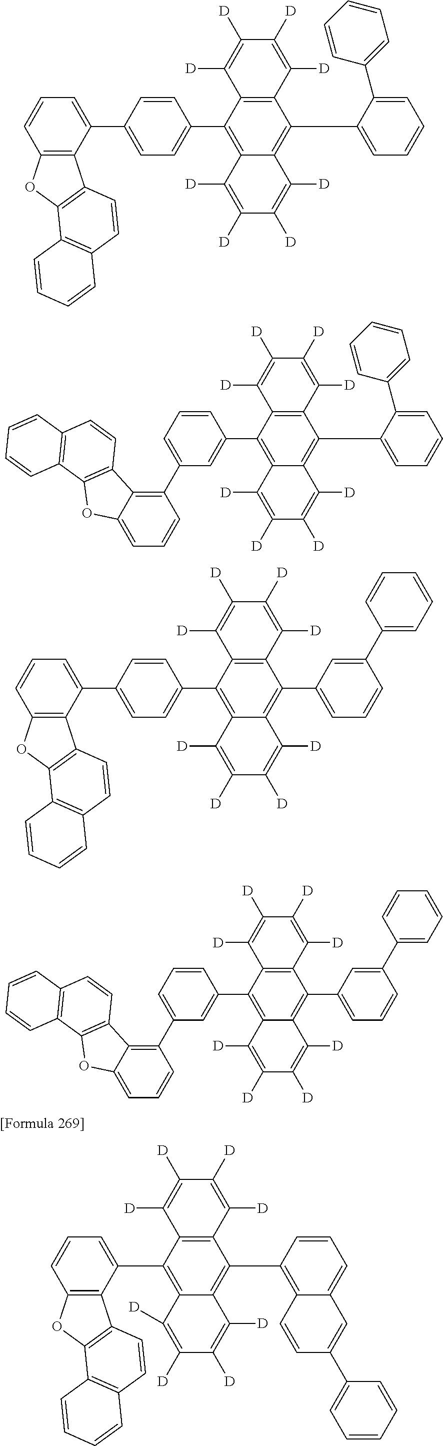

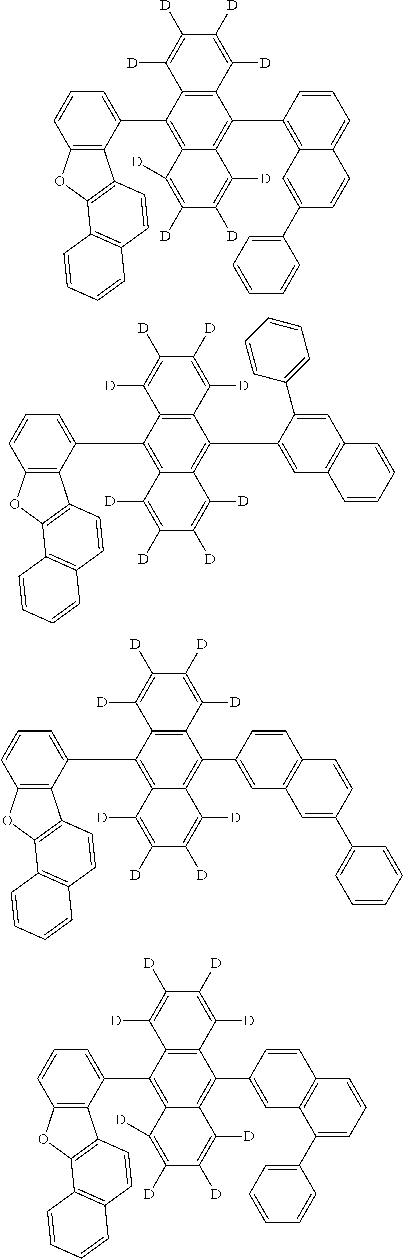

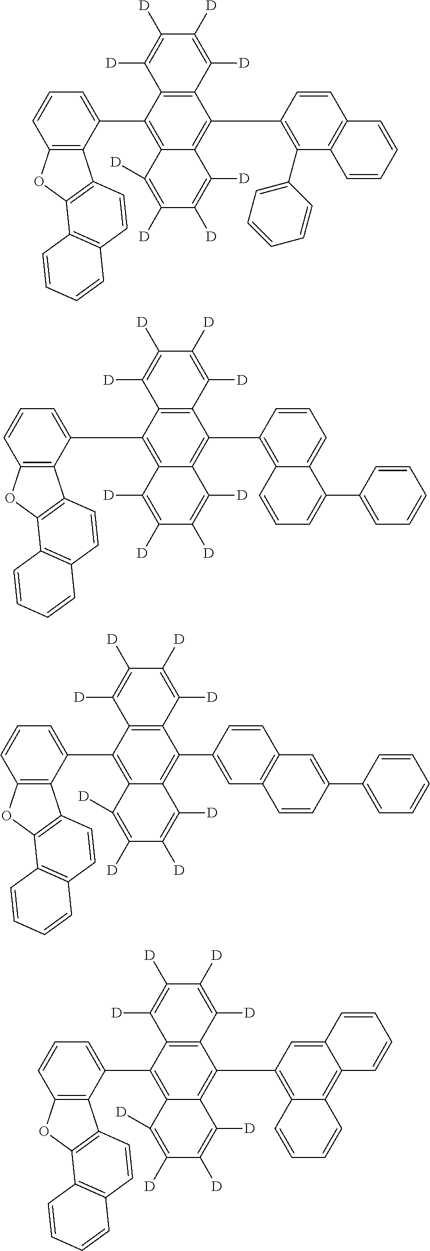 [SMILES: [2H]c1c([2H])c([2H])c2c(-c3cccc4oc5c6ccccc6ccc5c34)c3c([2H])c([2H])c([2H])c([2H])c3c(-c3ccc4ccccc4c3-c3ccccc3)c2c1[2H] ; [2H]c1c([2H])c([2H])c2c(-c3cccc4oc5c6ccccc6ccc5c34)c3c([2H])c([2H])c([2H])c([2H])c3c(-c3cccc4c(-c5ccccc5)cccc34)c2c1[2H] ; [2H]c1c([2H])c([2H])c2c(-c3cccc4oc5c6ccccc6ccc5c34)c3c([2H])c([2H])c([2H])c([2H])c3c(-c3ccc4cc(-c5ccccc5)ccc4c3)c2c1[2H] ; [2H]c1c([2H])c([2H])c2c(-c3cccc4oc5c6ccccc6ccc5c34)c3c([2H])c([2H])c([2H])c([2H])c3c(-c3cc4ccccc4c4ccccc34)c2c1[2H]]

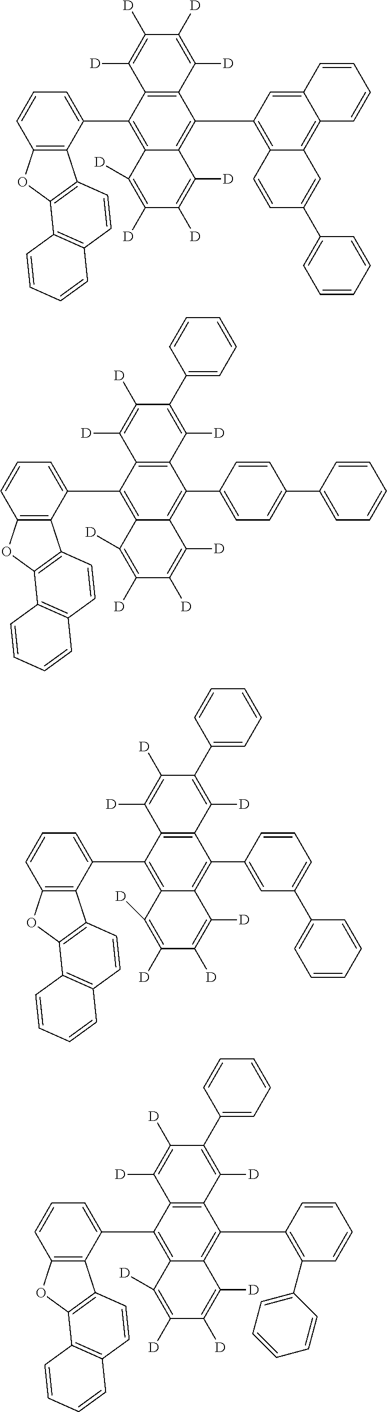 [SMILES: [2H]c1c([2H])c([2H])c2c(-c3cccc4oc5c6ccccc6ccc5c34)c3c([2H])c([2H])c([2H])c([2H])c3c(-c3cc4ccccc4c4cc(-c5ccccc5)ccc34)c2c1[2H] ; [2H]c1c([2H])c([2H])c2c(-c3cccc4oc5c6ccccc6ccc5c34)c3c([2H])c([2H])c(-c4ccccc4)c([2H])c3c(-c3ccc(-c4ccccc4)cc3)c2c1[2H] ; [2H]c1c([2H])c([2H])c2c(-c3cccc4oc5c6ccccc6ccc5c34)c3c([2H])c([2H])c(-c4ccccc4)c([2H])c3c(-c3cccc(-c4ccccc4)c3)c2c1[2H] ; [2H]c1c([2H])c([2H])c2c(-c3cccc4oc5c6ccccc6ccc5c34)c3c([2H])c([2H])c(-c4ccccc4)c([2H])c3c(-c3ccccc3-c3ccccc3)c2c1[2H]]

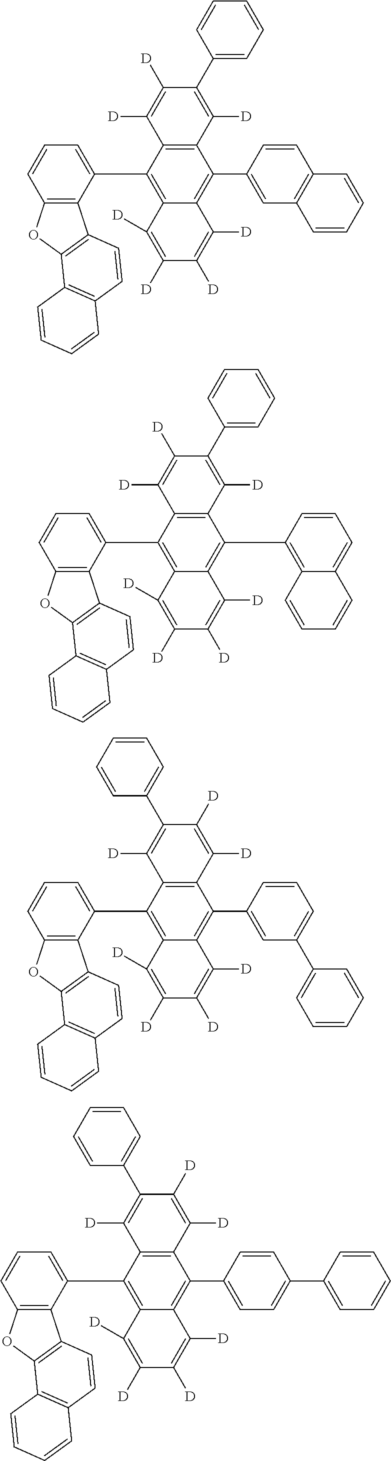

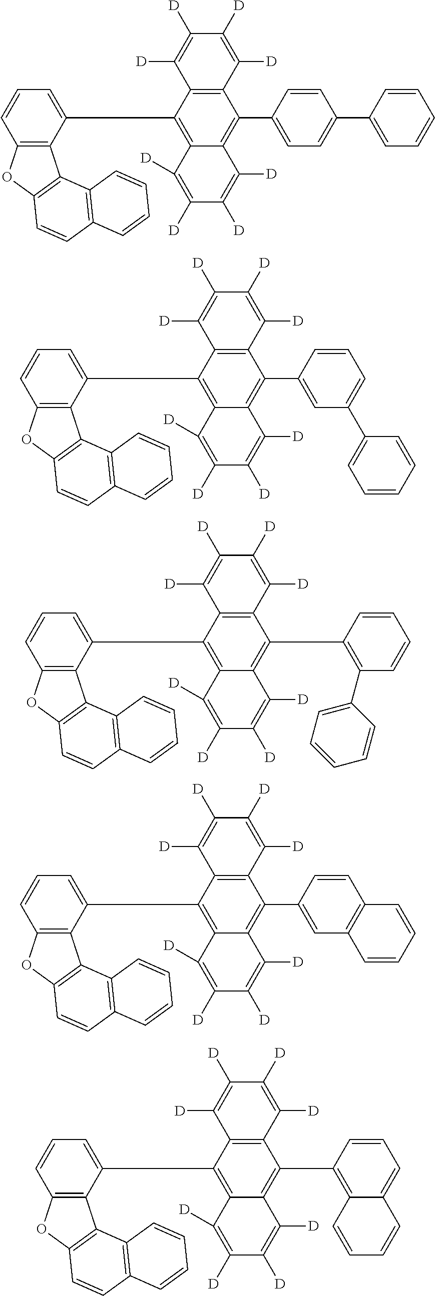

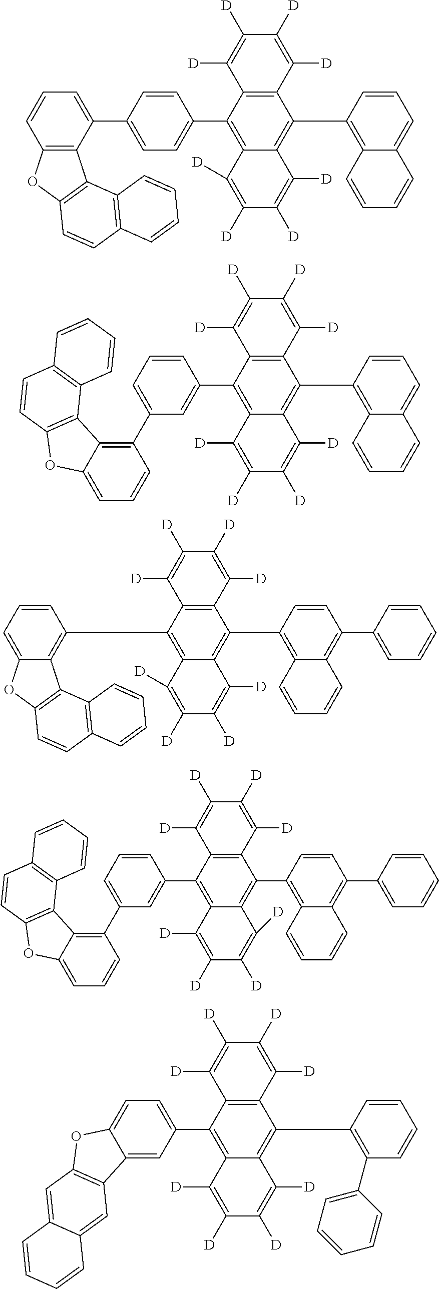

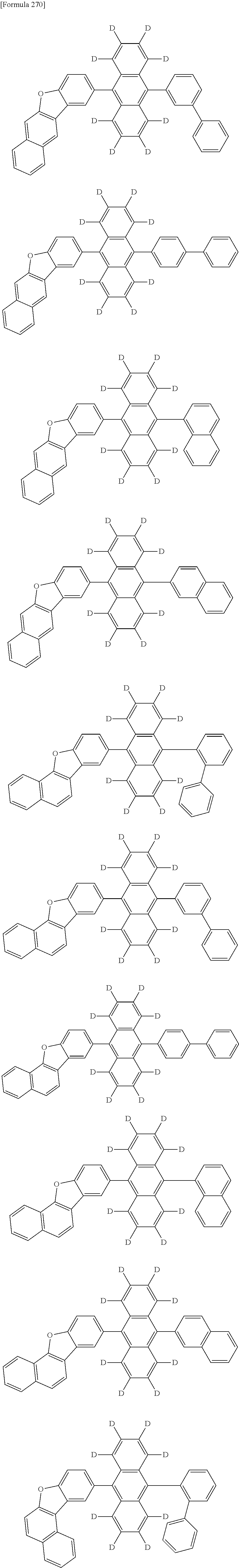

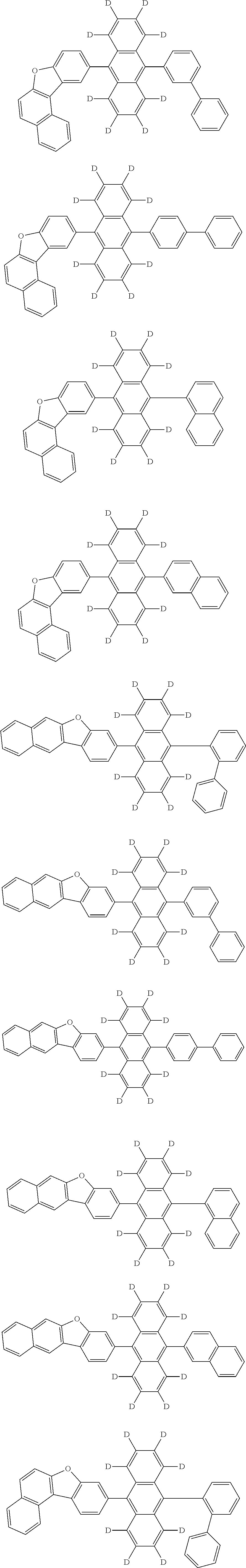

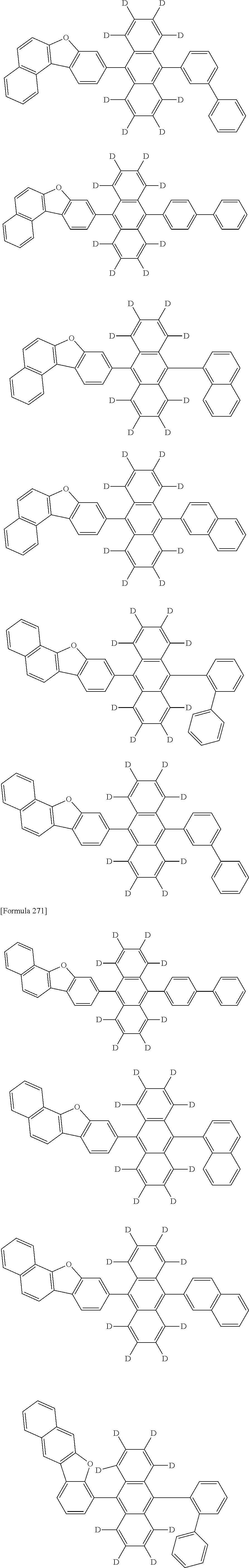

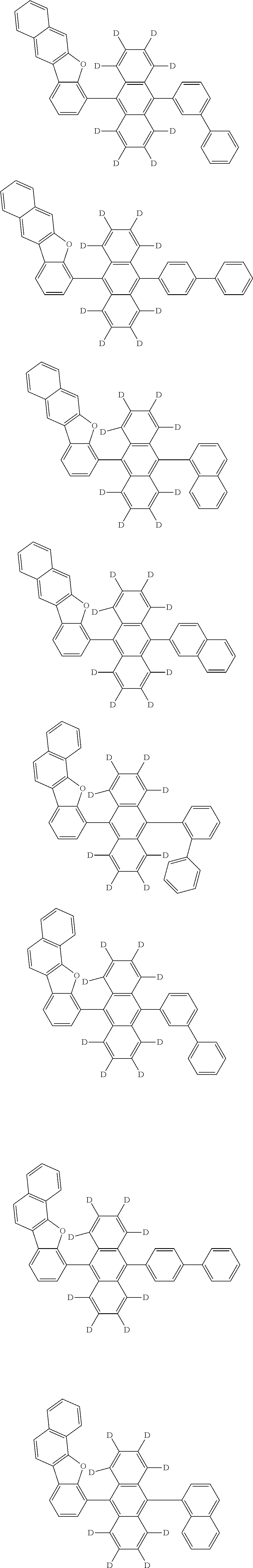

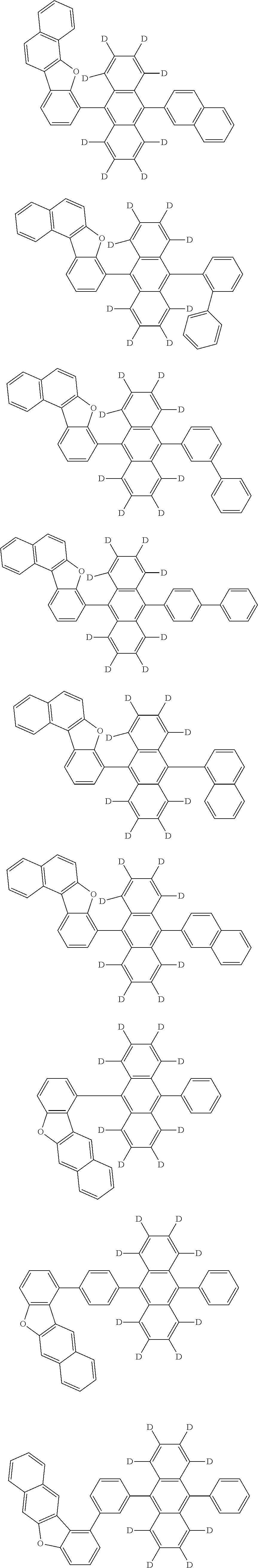

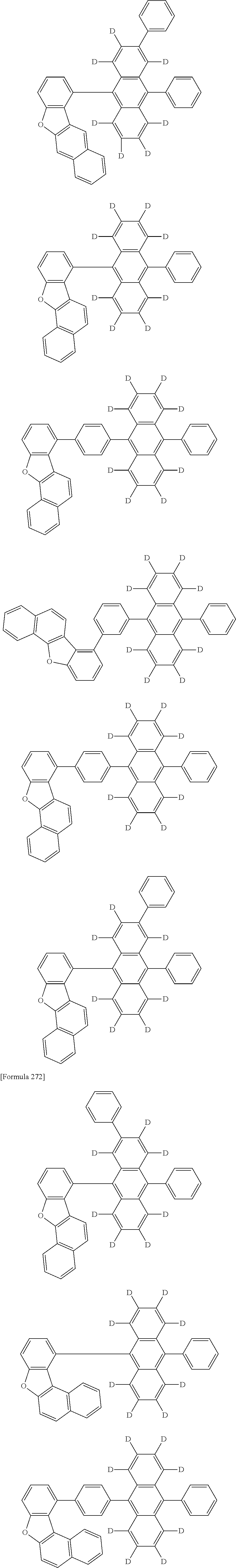

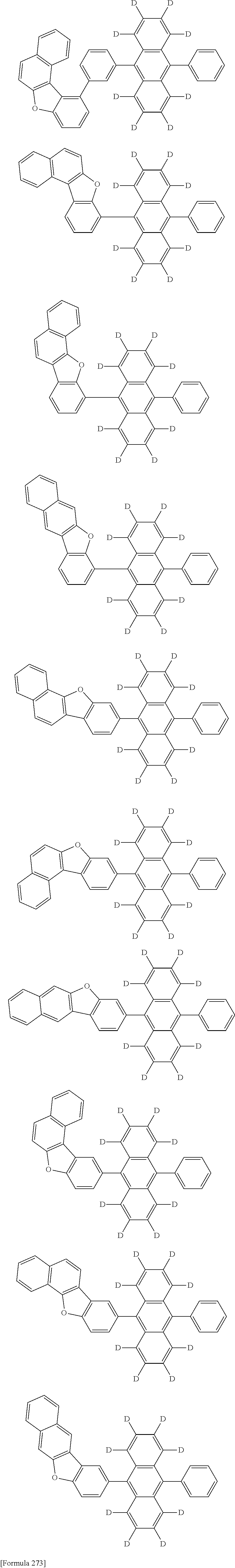

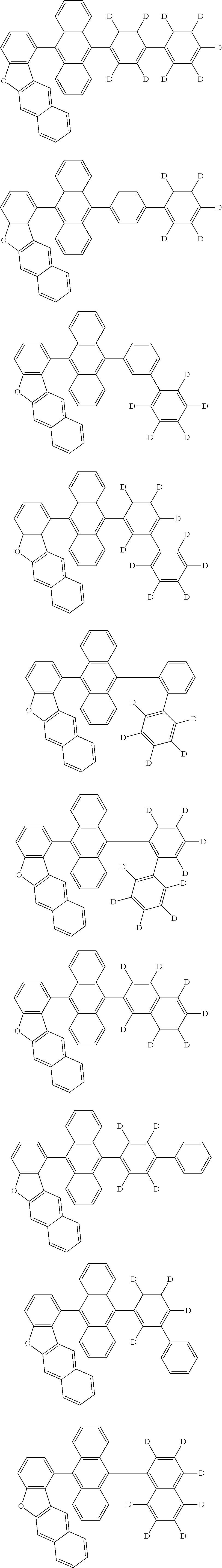

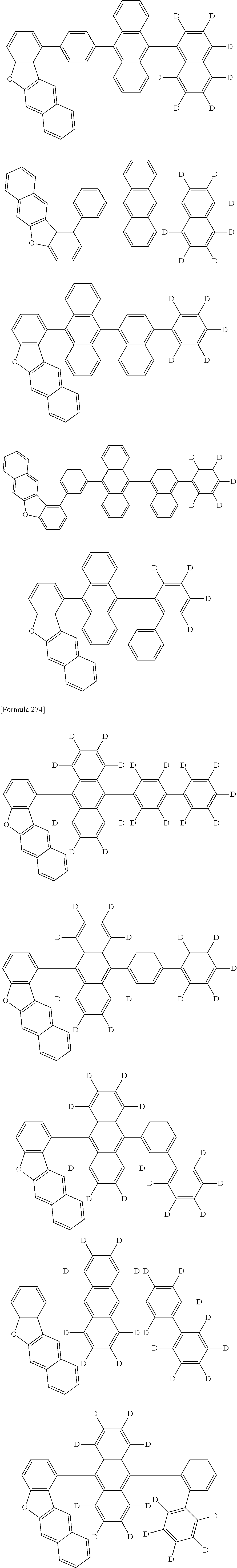

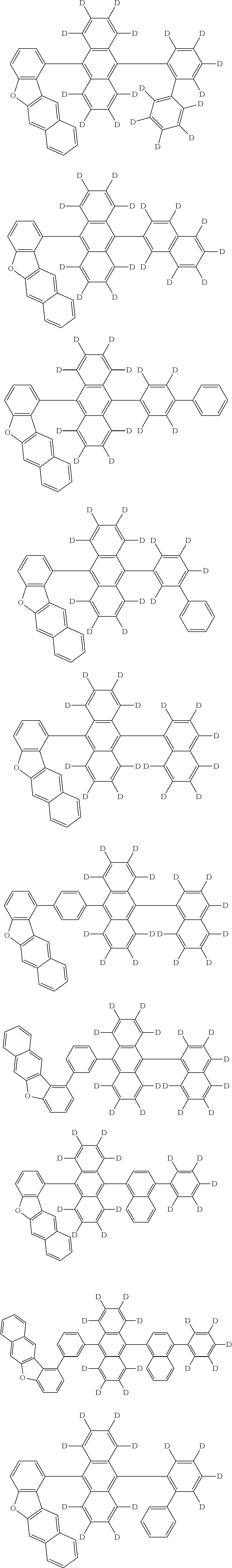

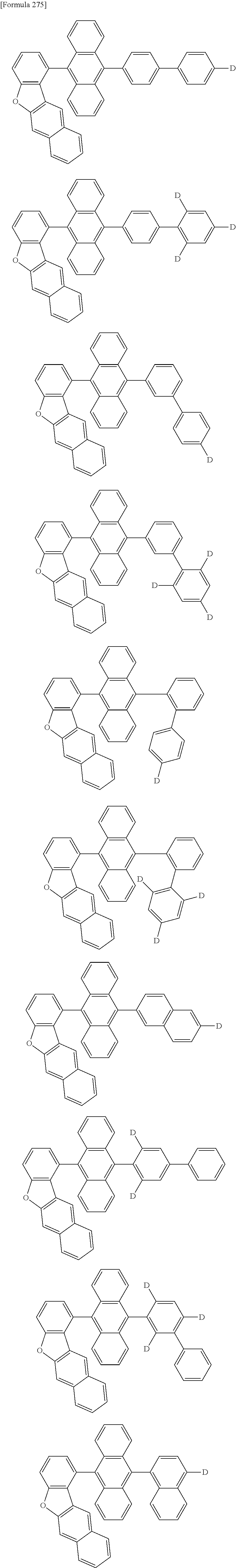

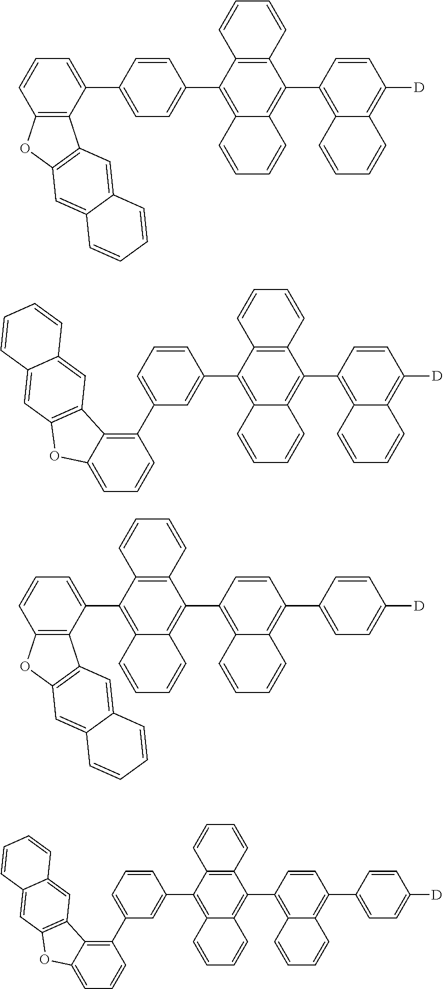

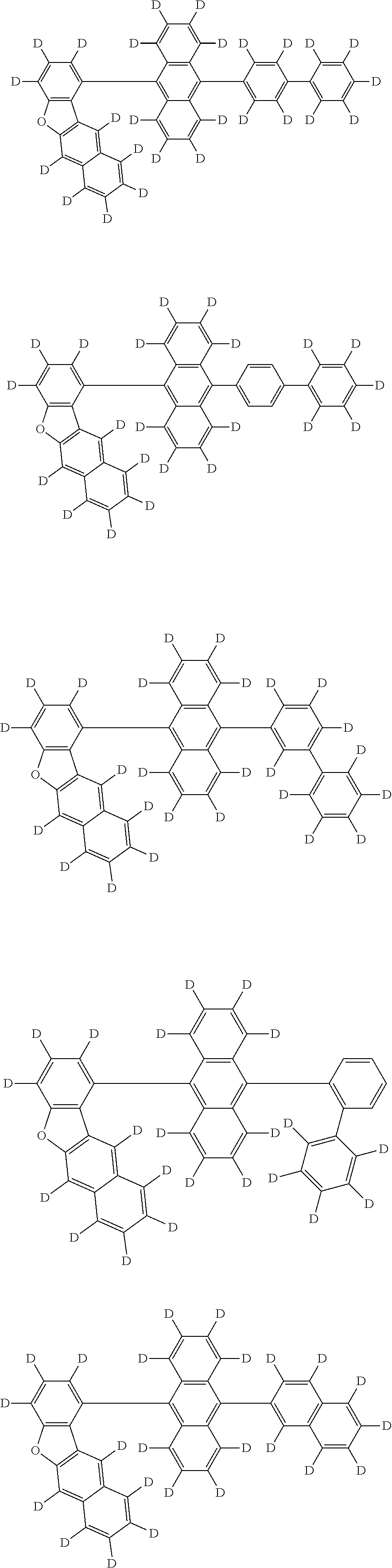

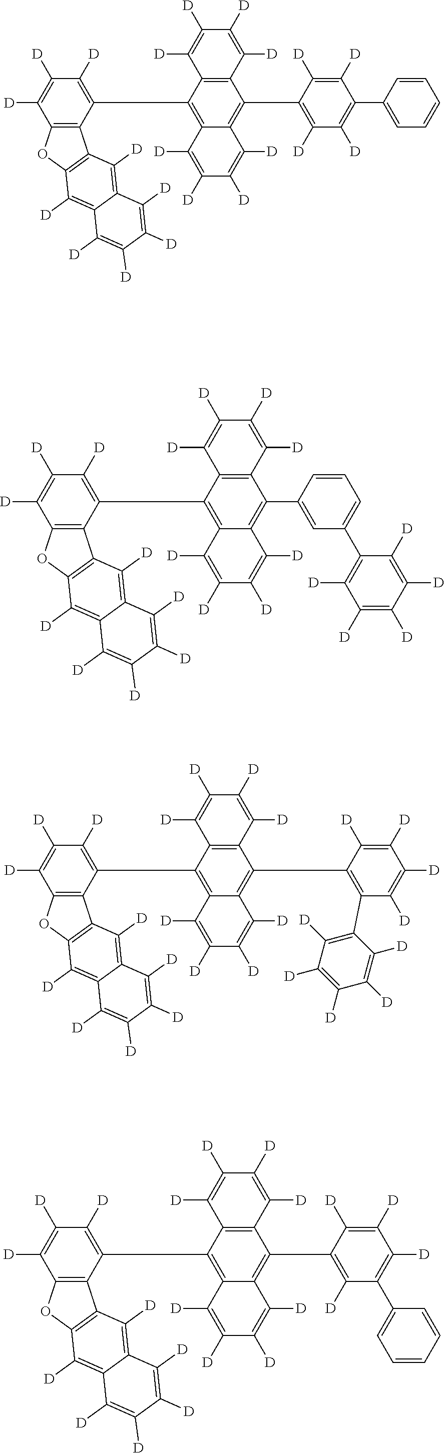

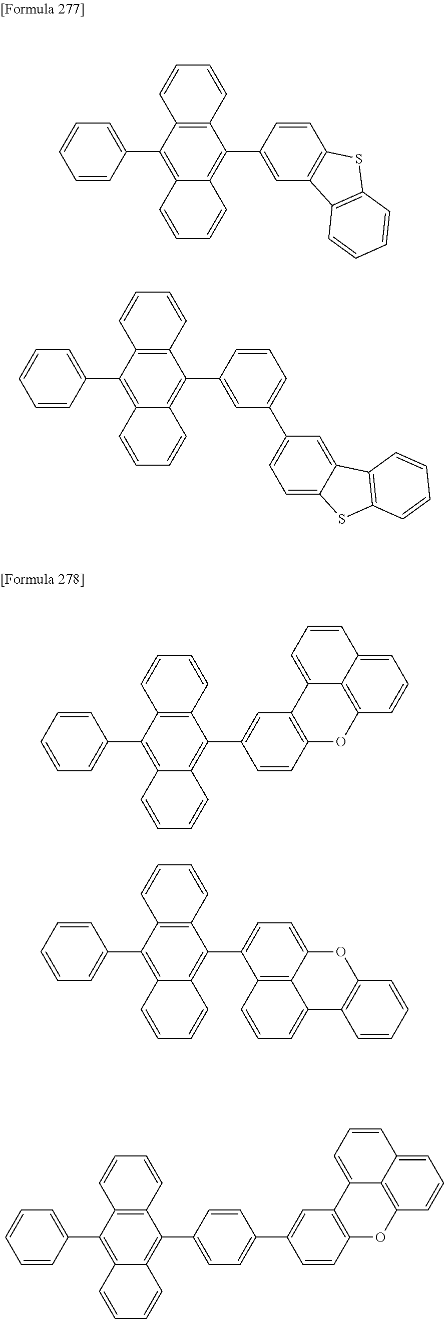

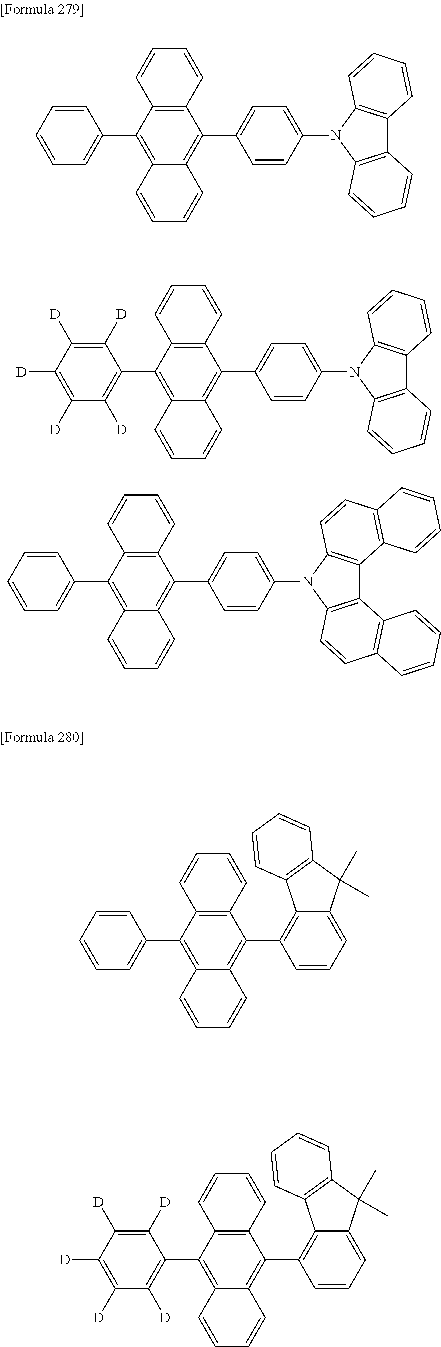

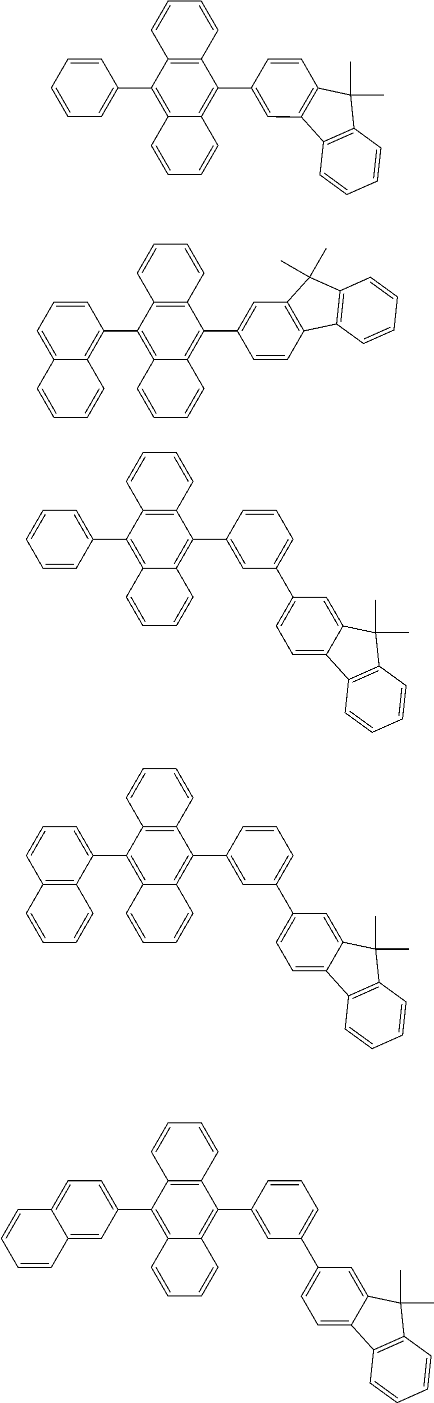

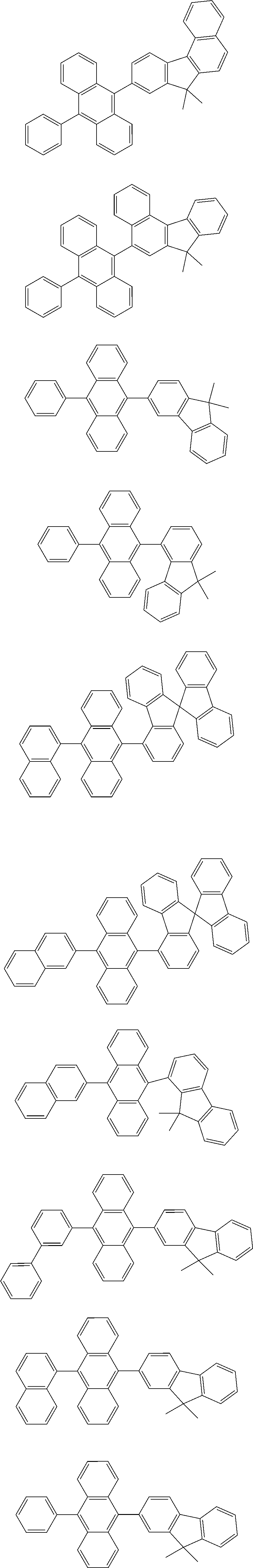

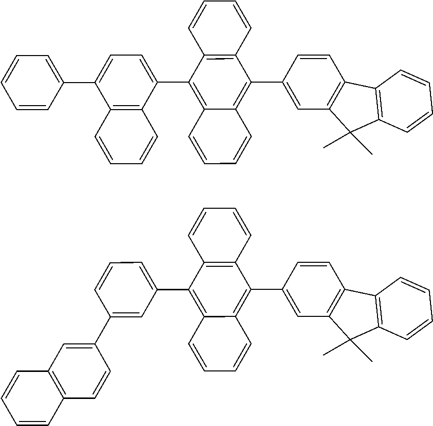

In specific examples of compounds below, D represents a deuterium atom; z, z1, z2, z3, z4, z5, and z6 each represent the number of deuterium atoms bonded to a ring; z is an integer in a range from 1 to 8; z1 is an integer in a range from 1 to 9; z2 to z5 each represent an integer in a range from 1 to 5; and z6 is an integer in a range from 1 to 7.

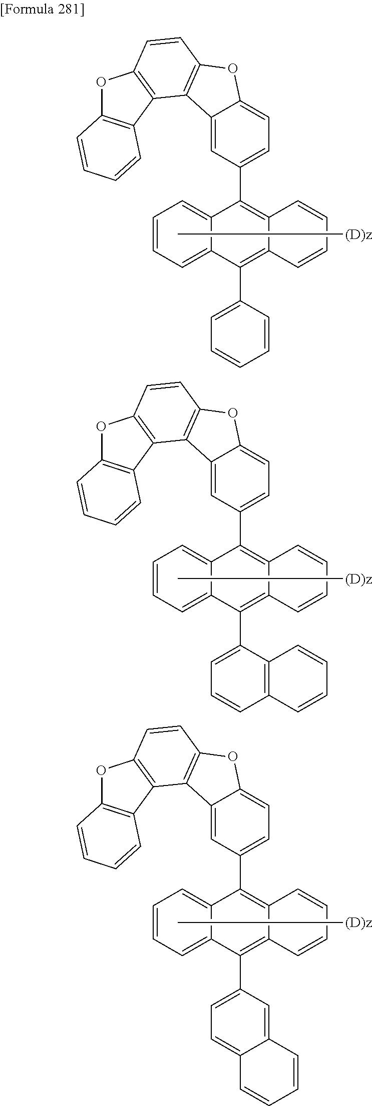

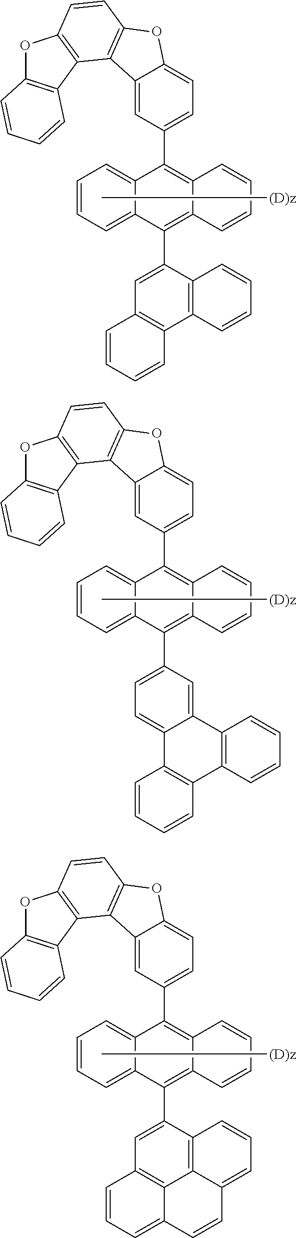

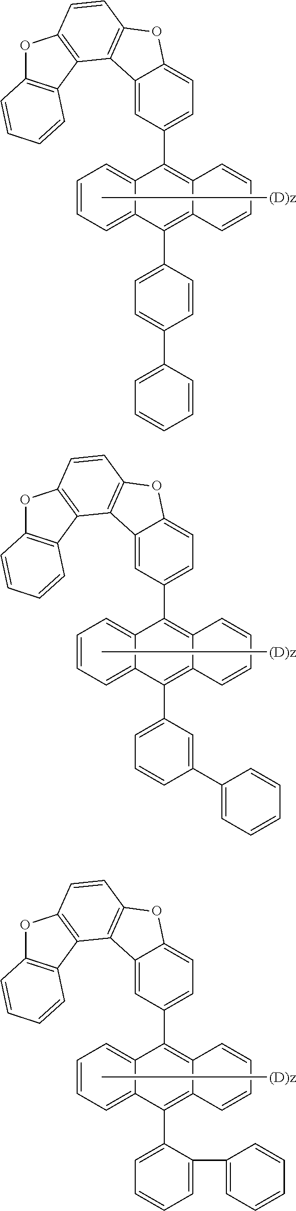

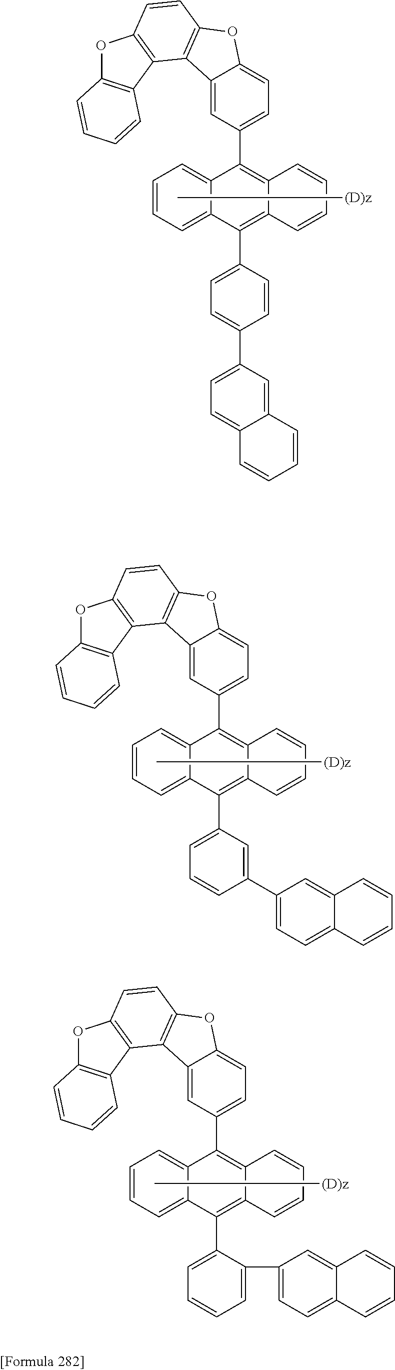

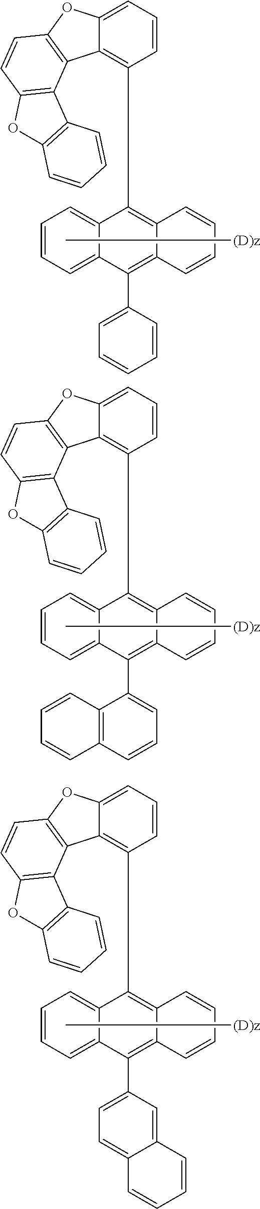

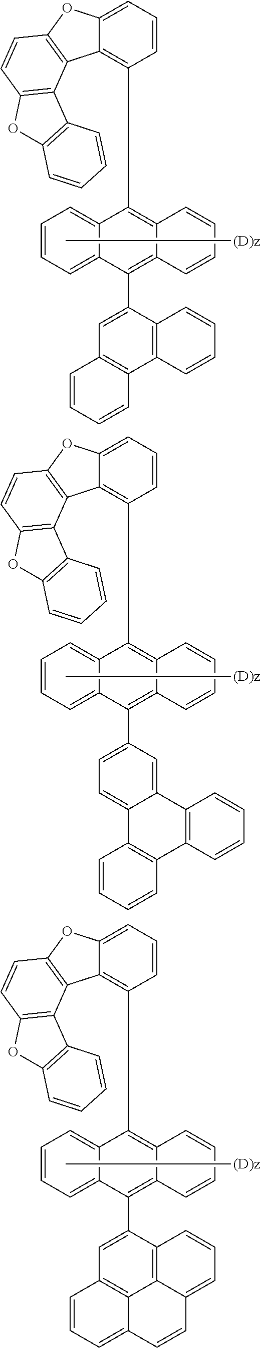

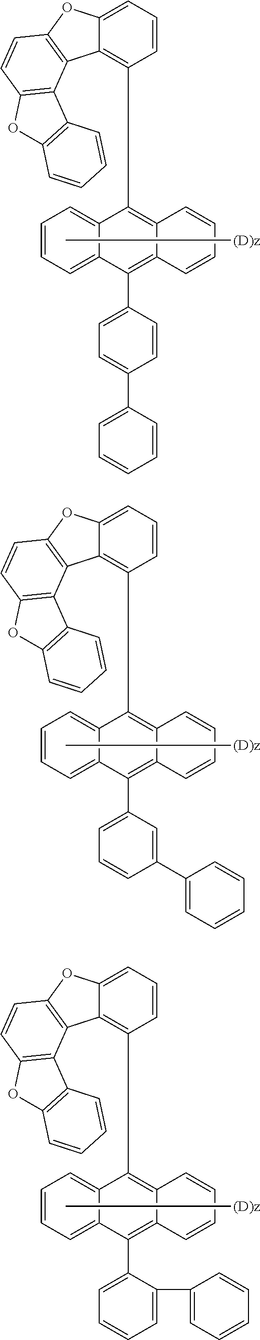

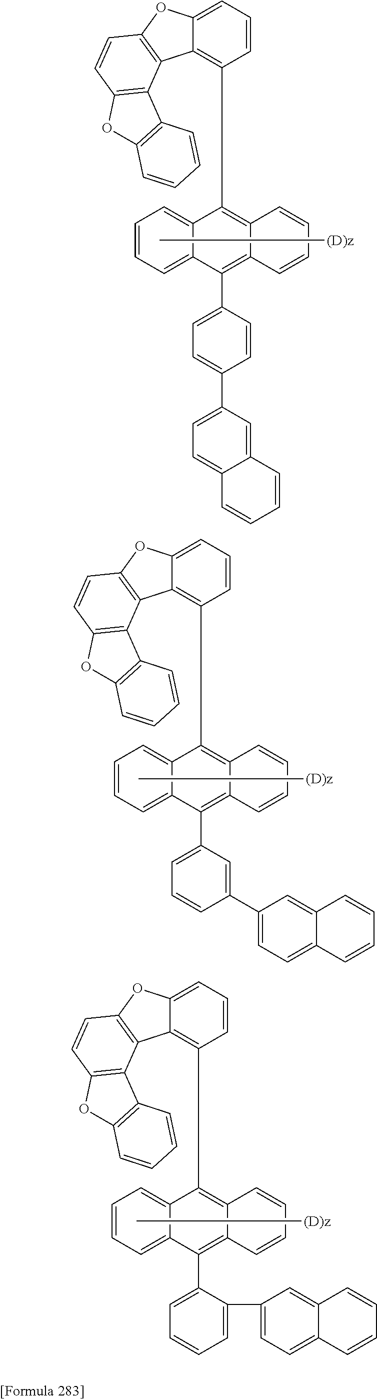

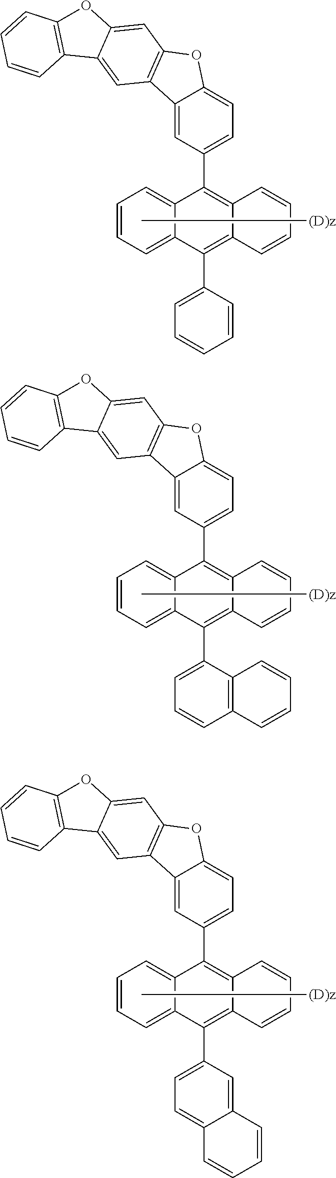

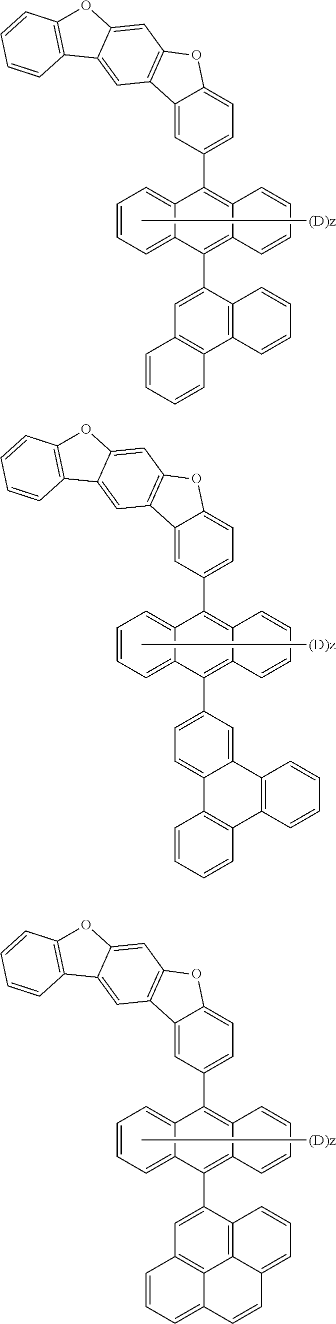

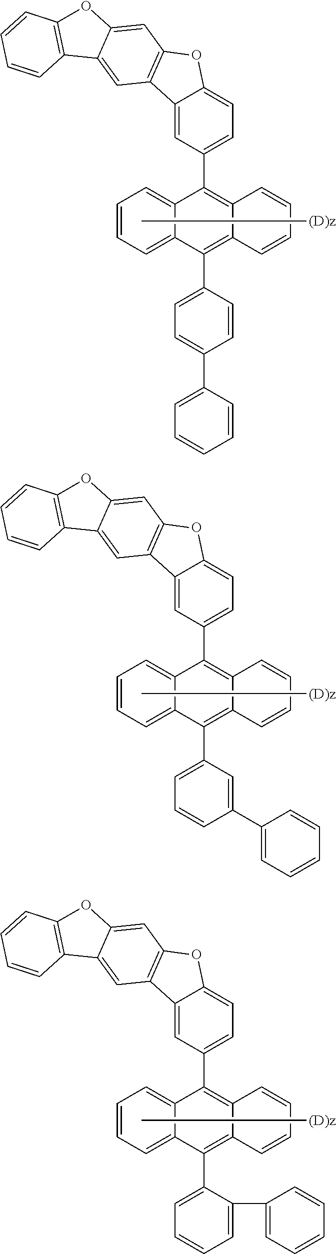

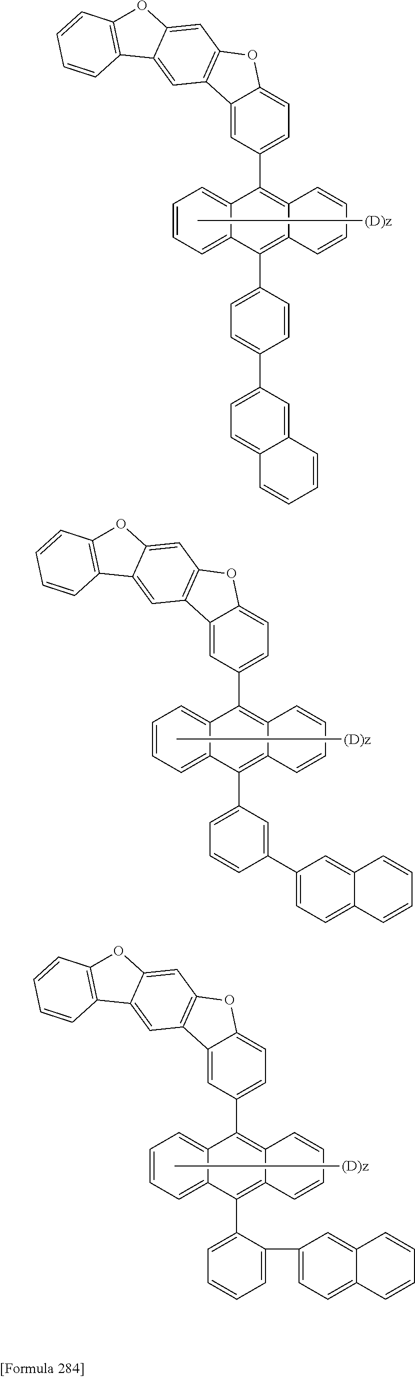

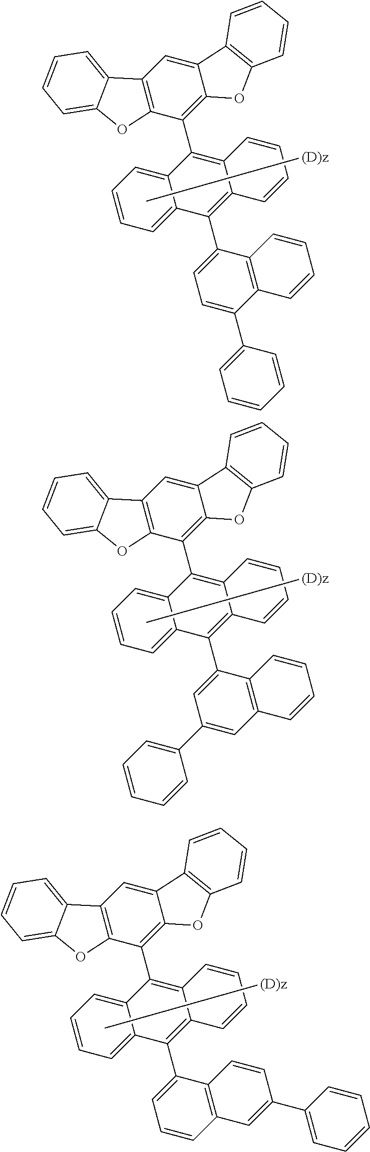

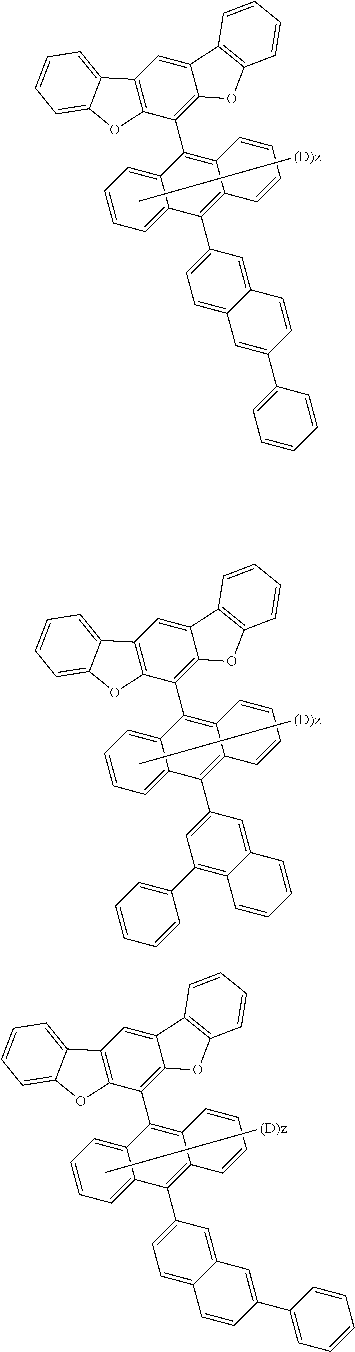

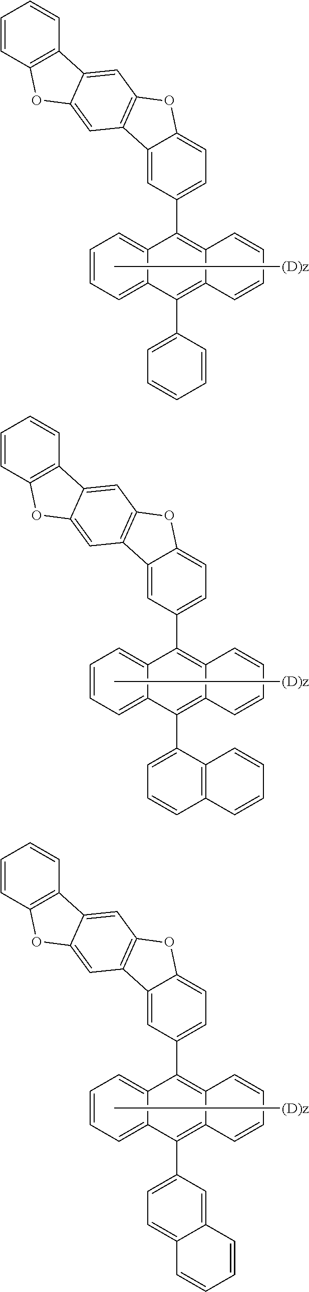

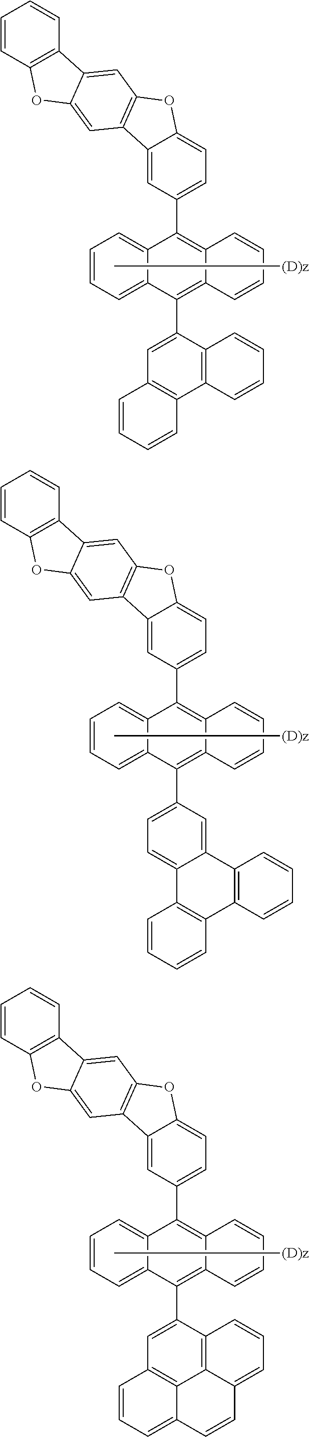

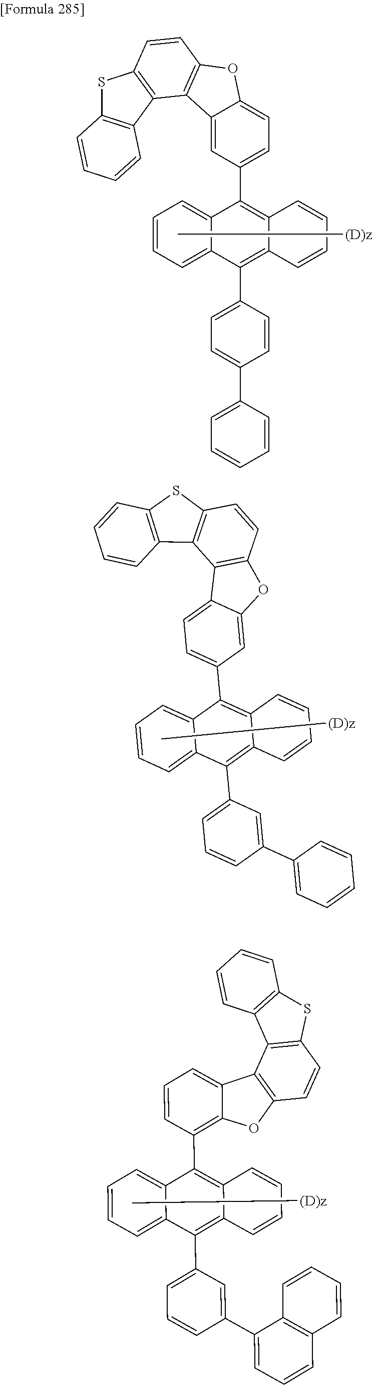

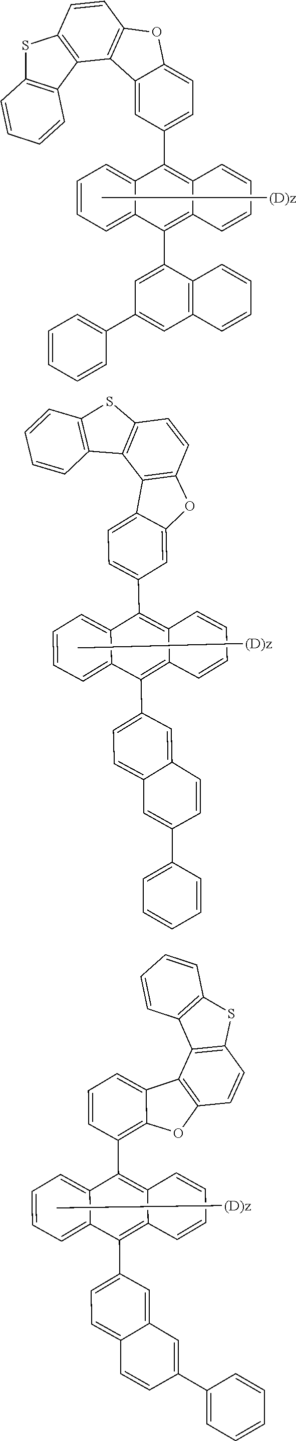

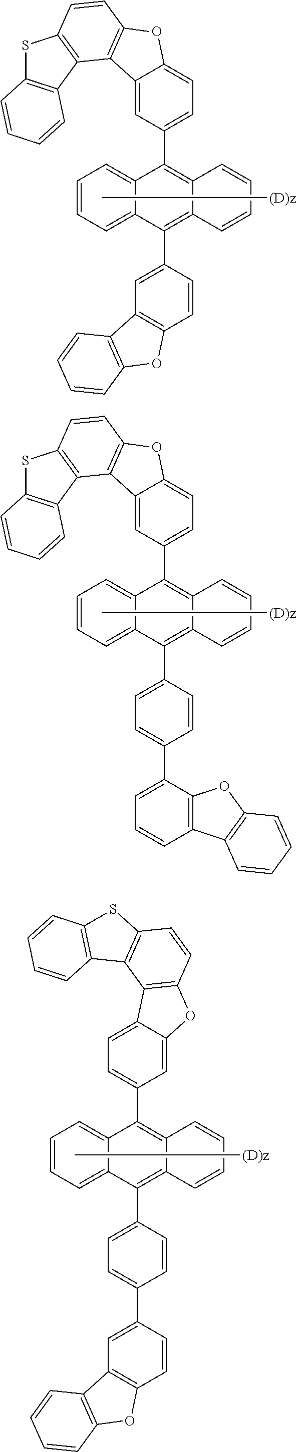

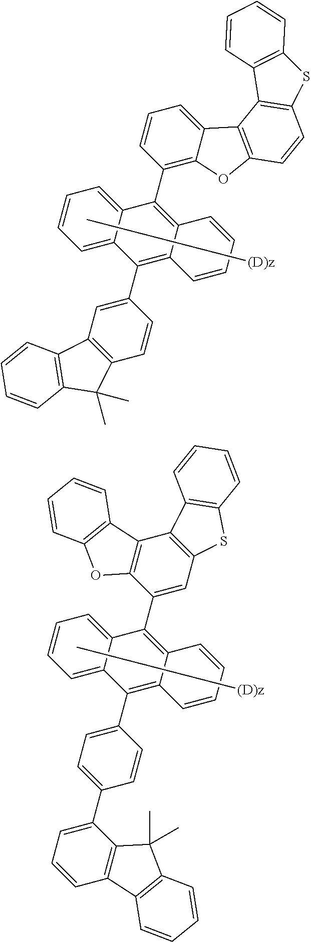

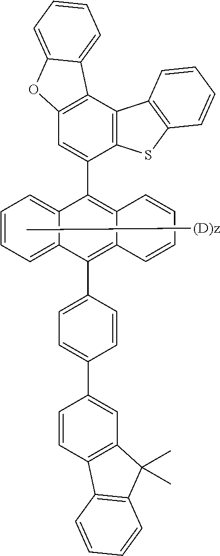

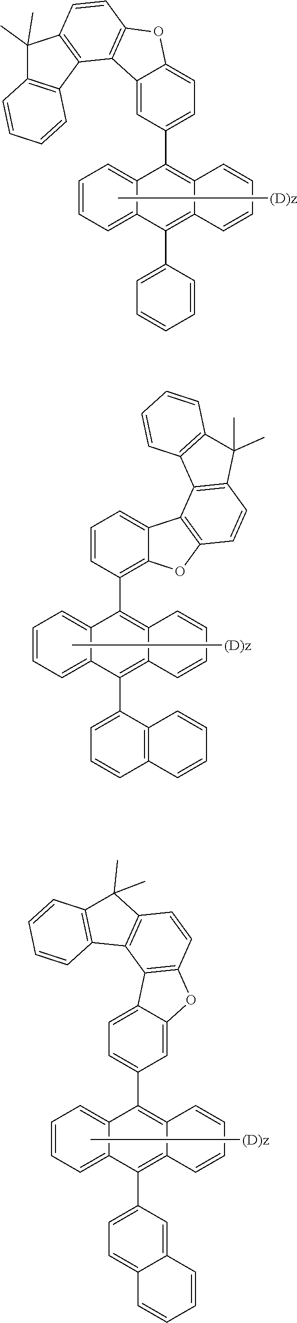

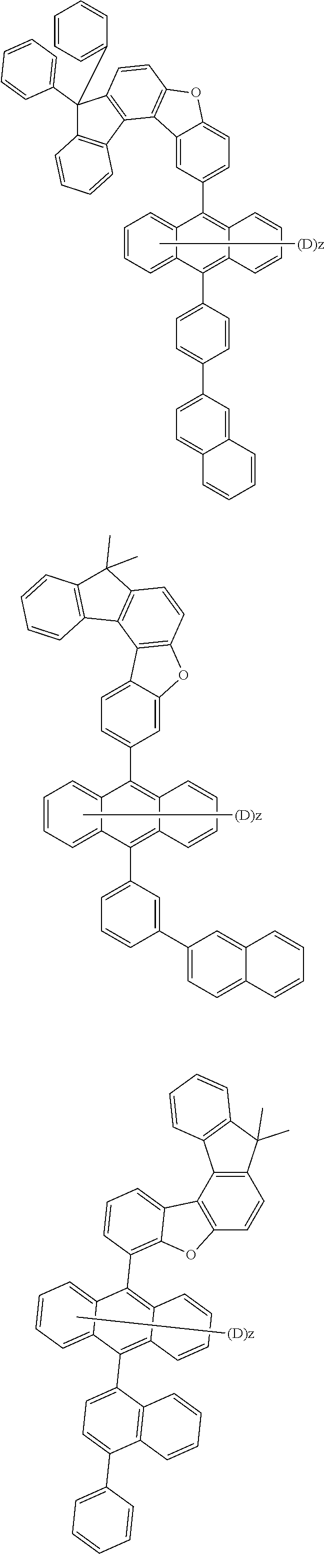

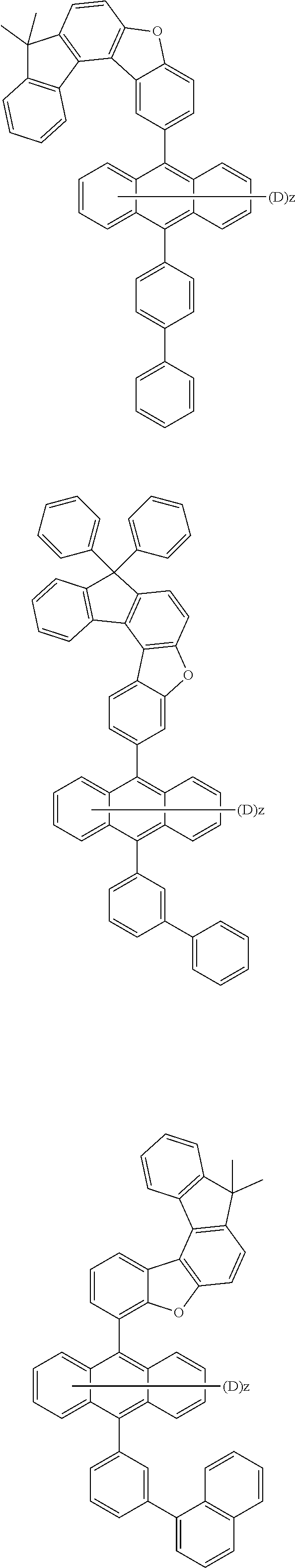

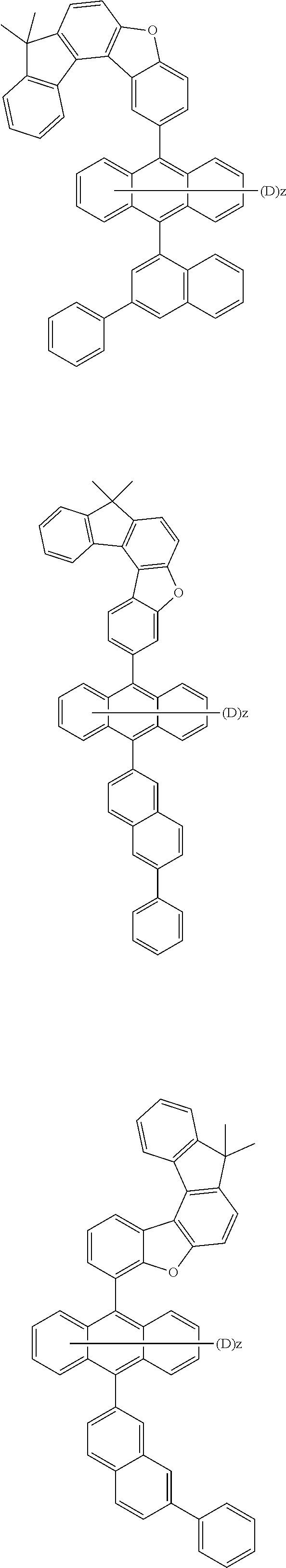

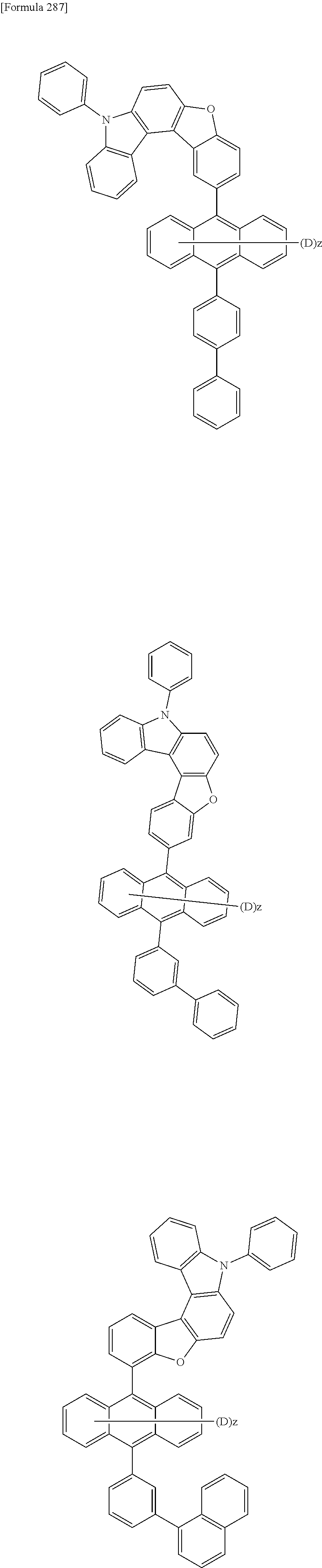

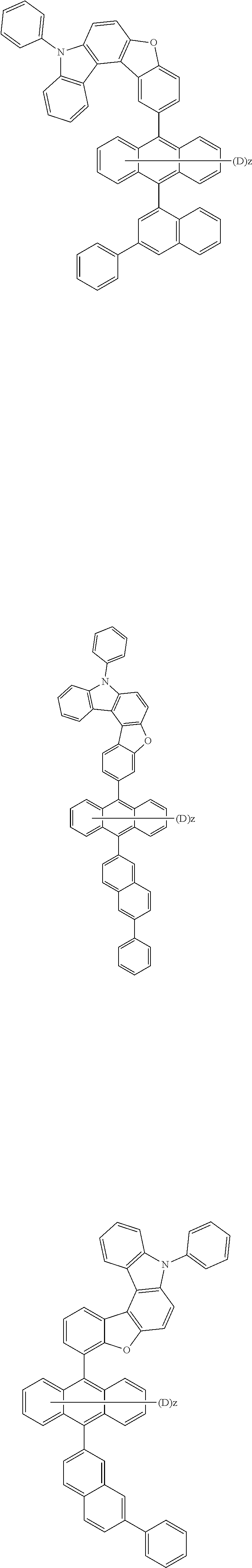

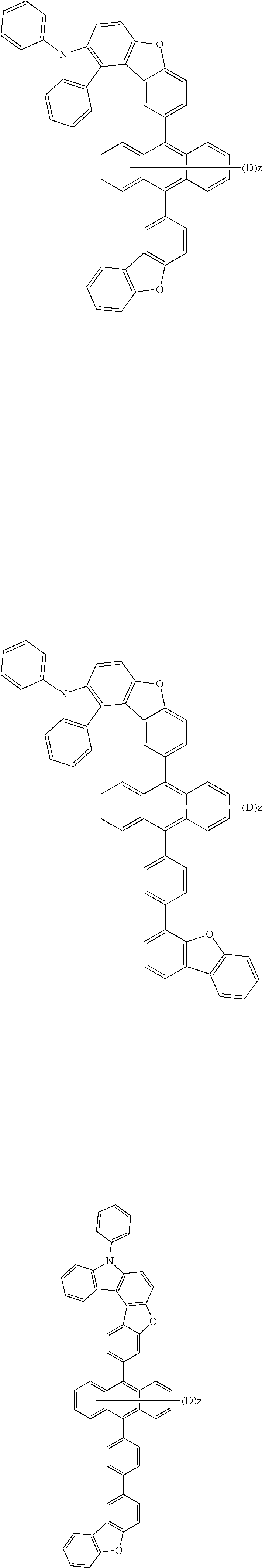

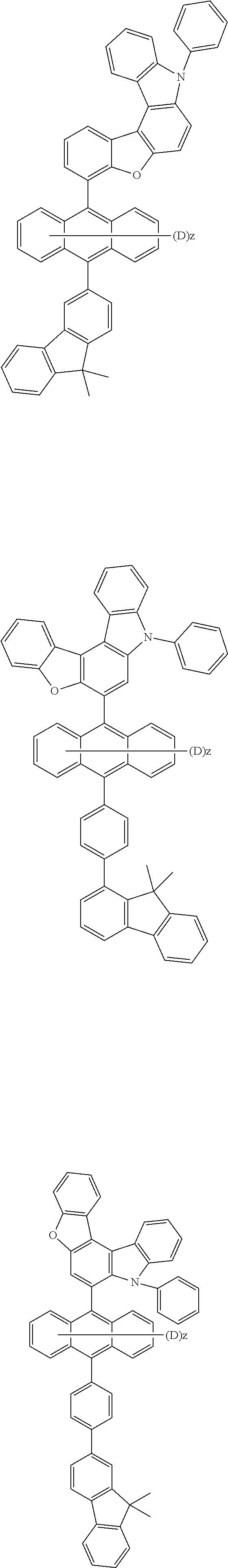

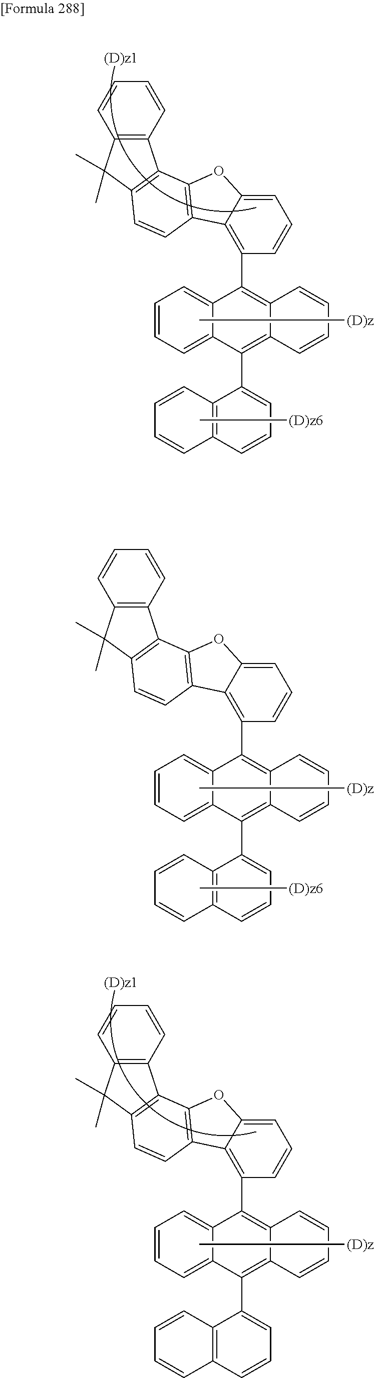

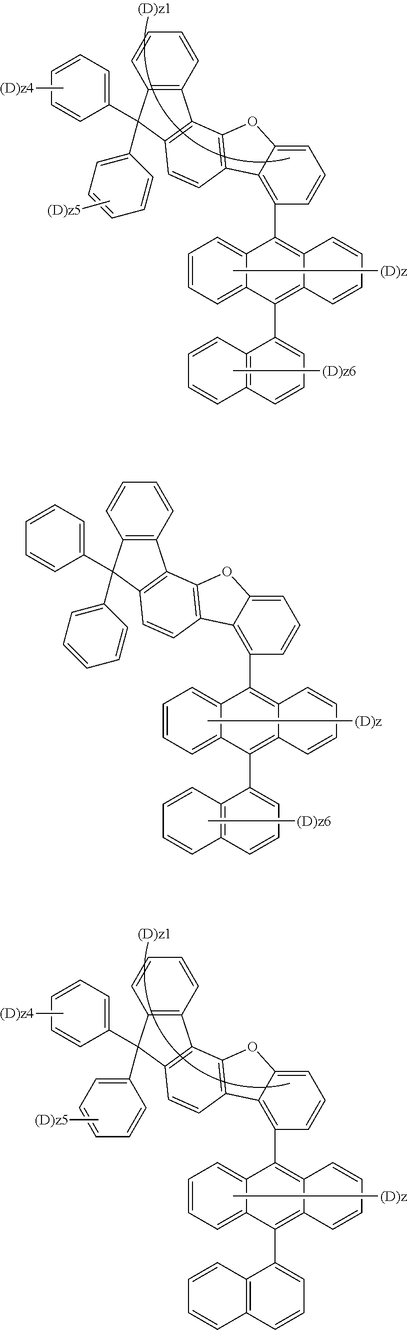

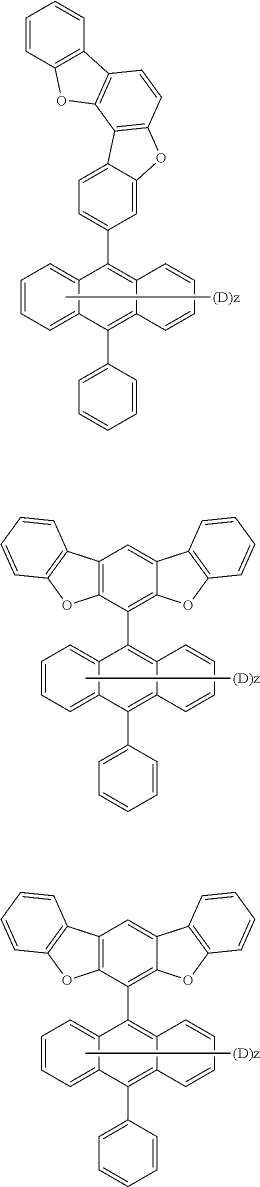

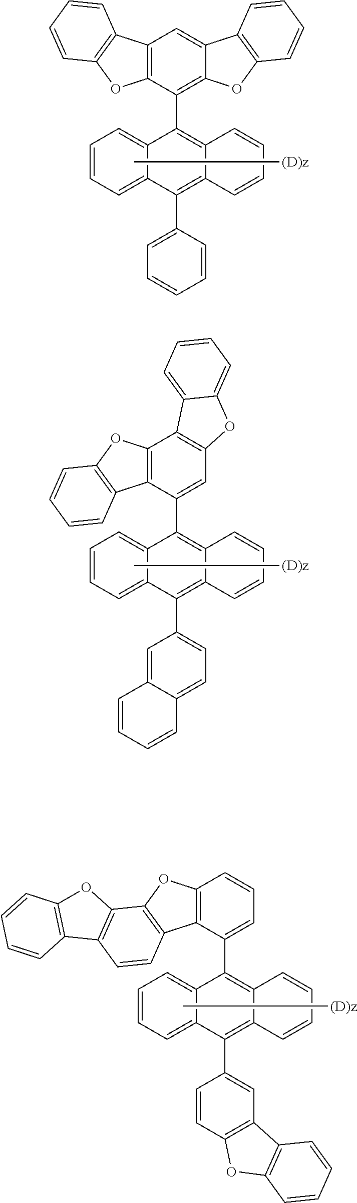

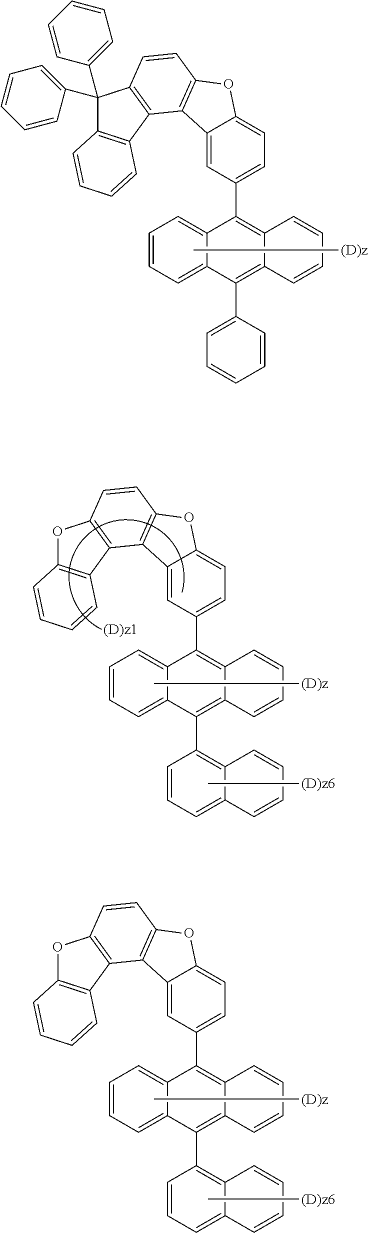

### Luminescent Compound

In an exemplary arrangement of the organic EL device according to the exemplary embodiment, the first emitting layer contains the first luminescent compound and the second emitting layer contains the second luminescent compound.

### Compound Represented by Formula (5)

In an exemplary arrangement of the organic EL device according to the exemplary embodiment, the luminescent compound is a compound represented by a formula (5) below.

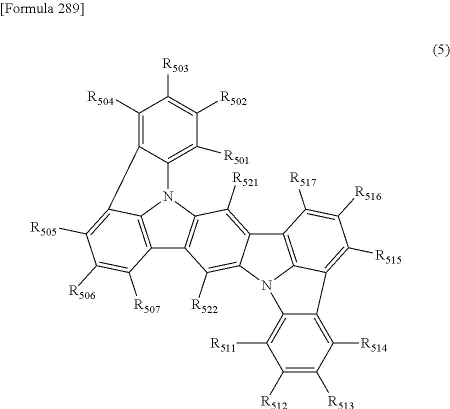

In the formula (5):
at least one combination of adjacent two or more of R₅₀₁ to R₅₀₇ and R₅₁₁ to R₅₁₇ are mutually bonded to form a substituted or unsubstituted monocyclic ring, mutually bonded to form a substituted or unsubstituted fused ring, or not mutually bonded,
R₅₀₁ to R₅₀₇ and R₅₁₁ to R₅₁₇ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₆), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
R₅₂₁ and R₅₂₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₆), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the luminescent compound, R₉₀₁, R₉₀₂, R₉₀₃, R₉₀₄, R₉₀₅, R₉₀₆ and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms, preferably, a substituted or unsubstituted alkyl group having 1 to 50 carbon atoms, or a substituted or unsubstituted aryl group having 6 to 50 ring carbon atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₆ are present, the plurality of R₉₀₆ are mutually the same or different;
when a plurality of R₉₀₆ are present, the plurality of R₉₀₆ are mutually the same or different; and when a plurality of R₉₀₇ are present, the plurality of R₉₀₇ are mutually the same or different.

“A combination of adjacent two or more of R₅₀₁ to R₅₀₇ and R₅₁₁ to R₅₁₇” refers to, for instance, a combination of R₅₀₁ and R₅₀₂, a combination of R₅₀₂ and R₅₀₃, a combination of R₅₀₃ and R₅₀₄, a combination of R₅₀₅ and R₅₀₆, a combination of R₅₀₆ and R₅₀₇, and a combination of R₅₀₁, R₅₀₂, and R₅₀₃.

In an exemplary embodiment, the compound represented by the formula (5) is a compound represented by a formula (52) below.

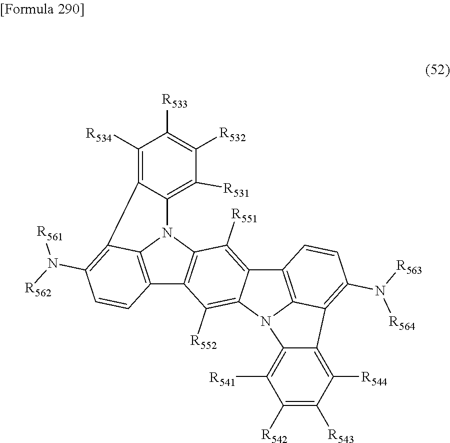

In the formula (52):
at least one combination of adjacent two or more of R₅₃₁ to R₅₃₄ and R₅₄₁ to R₅₄₄ are mutually bonded to form a substituted or unsubstituted monocyclic ring, mutually bonded to form a substituted or unsubstituted fused ring, or not mutually bonded;
R₅₃₁ to R₅₃₄ and R₅₄₁ to R₅₄₄ forming neither the substituted or unsubstituted monocyclic ring nor the substituted or unsubstituted fused ring, R₅₅₁, and R₅₅₂ are each independently a hydrogen atom, a substituted or unsubstituted aryl group having 6 to 50 ring carbon atoms, or a substituted or unsubstituted heterocyclic group having 5 to 50 ring atoms; and
R₅₆₁ to R₅₆₄ are each independently a substituted or unsubstituted aryl group having 6 to 50 ring carbon atoms, or a substituted or unsubstituted heterocyclic group having 5 to 50 ring atoms.

### Compound Represented by Formula (6)

In an exemplary arrangement of the organic EL device according to the exemplary embodiment, the luminescent compound is a compound represented by a formula (6) below.

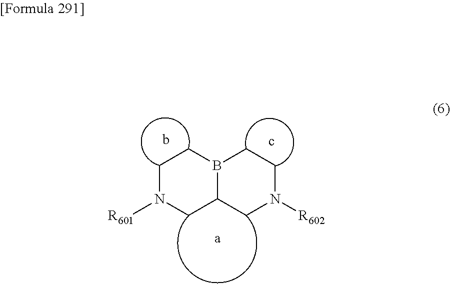

In the formula (6):
a ring a, a ring b, and a ring c are each independently a substituted or unsubstituted aromatic hydrocarbon ring having 6 to 50 ring carbon atoms, or a substituted or unsubstituted heterocycle having 5 to 50 ring atoms;
R₆₀₁ and R₆₀₂ are each independently bonded to the ring a, the ring b or the ring c to form a substituted or unsubstituted heterocycle, or not bonded thereto to form no substituted or unsubstituted heterocycle; and
R₆₀₁ and R₆₀₂ not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In an exemplary arrangement of the organic EL device according to the exemplary embodiment, the ring a, ring b and ring c are each a ring (a substituted or unsubstituted aromatic hydrocarbon ring having 6 to 50 ring carbon atoms, or a substituted or unsubstituted heterocycle having 5 to 50 ring atoms) fused with a fused bicyclic structure formed of a boron atom and two nitrogen atoms at the center of the formula (6).

The “aromatic hydrocarbon ring” for the rings a, b, and c has the same structure as a compound formed by introducing a hydrogen atom to the “aryl group”.

Ring atoms of the “aromatic hydrocarbon ring” for the ring a include three carbon atoms on the fused bicyclic structure at the center of the formula (6).

Ring atoms of the “aromatic hydrocarbon ring” for the rings b and c include two carbon atoms on the fused bicyclic structure at the center of the formula (6).

Specific examples of the “substituted or unsubstituted aromatic hydrocarbon ring having 6 to 50 ring carbon atoms” include a compound formed by introducing a hydrogen atom to the “aryl group” described in the specific example group G1.

The “heterocycle” for the rings a, b, and c has the same structure as a compound formed by introducing a hydrogen atom to the “heterocyclic group” described above.

Ring atoms of the “heterocycle” for the ring a include three carbon atoms on the fused bicyclic structure at the center of the formula (6). Ring atoms of the “heterocycle” for the rings b and c include two carbon atoms on the fused bicyclic structure at the center of the formula (6). Specific examples of the “substituted or unsubstituted heterocycle having 5 to 50 ring atoms” include a compound formed by introducing a hydrogen atom to the “heterocyclic group” described in the specific example group G2.

R₆₀₁ and R₆₀₂ may be each independently bonded to the ring a, the ring b, or the ring c to form a substituted or unsubstituted heterocycle. The “heterocycle” in this arrangement includes a nitrogen atom on the fused bicyclic structure at the center of the formula (6). The heterocycle in the above arrangement optionally includes a hetero atom other than the nitrogen atom. R₆₀₁ and R₆₀₂ being bonded to the ring a, ring b, or ring c specifically means that atoms forming R₆₀₁ and R₆₀₂ are bonded to atoms forming the ring a, ring b, or ring c. For instance, R₆₀₁ may be bonded with the ring a to form a bicyclic (or tri-or-more cyclic) fused nitrogen-containing heterocycle, in which the ring including R₆₀₁ and the ring a are fused. Specific examples of the nitrogen-containing heterocycle include a compound corresponding to the nitrogen-containing bi(or-more)cyclic fused heterocyclic group in the specific example group G2.

The same applies to R₆₀₁ bonded with the ring b, R₆₀₂ bonded with the ring a, and R₆₀₂ bonded with the ring c.

Optionally, R₆₀₁ and R₆₀₂ are each independently not bonded with the ring a, ring b, or ring c.

In an exemplary embodiment, the ring a, ring b and ring c in the formula (6) are each independently a substituted or unsubstituted aromatic hydrocarbon ring having 6 to 50 ring carbon atoms.

In an exemplary embodiment, the ring a, ring b and ring c in the formula (6) are each independently a substituted or unsubstituted benzene ring or a substituted or unsubstituted naphthalene ring.

In an exemplary embodiment, R₆₀₁ and R₆₀₂ in the formula (6) are each independently a substituted or unsubstituted aryl group having 6 to 50 ring carbon atoms, or a substituted or unsubstituted heterocyclic group having 5 to 50 ring atoms, preferably, a substituted or unsubstituted aryl group having 6 to 50 ring carbon atoms.

In an exemplary embodiment, the compound represented by the formula (6) is a compound represented by a formula (62) below.

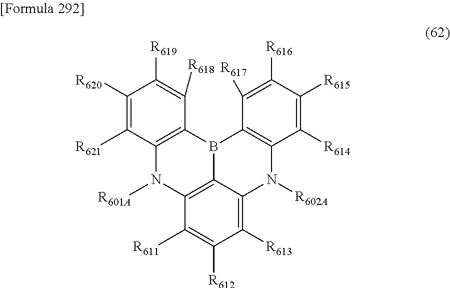

In the formula (62):
R_{601A} is bonded with at least one of R₆₁₁ or R₆₂₁ to form a substituted or unsubstituted heterocycle, or not bonded therewith to form no substituted or unsubstituted heterocycle;
R_{602A} is bonded with at least one of R₆₁₃ or R₆₁₄ to form a substituted or unsubstituted heterocycle, or not bonded therewith to form no substituted or unsubstituted heterocycle;
R_{601A} and R_{602A} not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
at least one combination of adjacent two or more of R₆₁₁ to R₆₂₁ are mutually bonded to form a substituted or unsubstituted monocyclic ring, mutually bonded to form a substituted or unsubstituted fused ring, or not mutually bonded; and
R₆₁₁ to R₆₂₁ forming neither the substituted or unsubstituted heterocycle nor the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (62), R₉₀₁, R₉₀₂, R₉₀₃, R₉₀₄, R₉₀₅, R₉₀₆, and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

R_{601A} and R_{602A} in the formula (62) are groups corresponding to R₆₀₁ and R₆₀₂ in the formula (6), respectively.

For instance, R_{601A} and R₆₁₁ are optionally bonded with each other to form a bicyclic (or tri-or-more cyclic) fused nitrogen-containing heterocycle, in which the ring including R_{601A} and R₆₁₁ and a benzene ring corresponding to the ring a are fused. Specific examples of the nitrogen-containing heterocycle include a compound corresponding to the nitrogen-containing bi(or-more)cyclic fused heterocyclic group in the specific example group G2. The same applies to R_{601A} bonded with R₆₂₁, R_{602A} bonded with R₆₁₃, and R_{602A} bonded with R₆₁₄.

At least one combination of adjacent two or more of R₆₁₁ to R₆₂₁ are mutually bonded to form a substituted or unsubstituted monocyclic ring, or mutually bonded to form a substituted or unsubstituted fused ring.

For instance, R₆₁₁ and R₆₁₂ are optionally mutually bonded to form a structure in which a benzene ring, indole ring, pyrrole ring, benzofuran ring, benzothiophene ring or the like is fused to the six-membered ring bonded with R₆₁₁ and R₆₁₂, the resultant fused ring forming a naphthalene ring, carbazole ring, indole ring, dibenzofuran ring, or dibenzothiophene ring.

In an exemplary embodiment, the compound represented by the formula (6) is a compound represented by a formula (42-2) below.

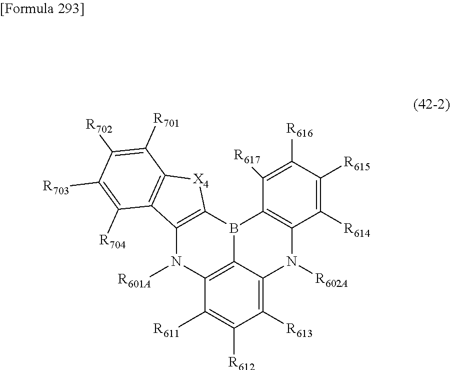

In the formula (42-2), R₆₁₁ to R₆₁₇, R_{601A} and R_{602A} each independently represent the same as R₆₁₁ to R₆₁₇, R_{601A} and R_{602A} in the formula (62);
X₄ is an oxygen atom or a sulfur atom;
at least one combination of adjacent two or more of R₇₀₁ to R₇₀₄ are mutually bonded to form a substituted or unsubstituted monocyclic ring, mutually bonded to form a substituted or unsubstituted fused ring, or not mutually bonded;
R₇₀₁ to R₇₀₄ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms or a substituted or unsubstituted heterocyclic group having 5 to 50 ring atoms; and
in the formula (42-2), R₉₀₁, R₉₀₂, R₉₀₃, R₉₀₄, R₉₀₅, R₉₀₆ and R₉₀₇ each independently represent the same as R₉₀₁, R₉₀₂, R₉₀₃, R₉₀₄, R₉₀₅, R₉₀₆ and R₉₀₇ in the formula (62).

### Compound Represented by Formula (3A)

In an exemplary arrangement of the organic EL device according to the exemplary embodiment, the luminescent compound is a compound represented by a formula (3A) below.

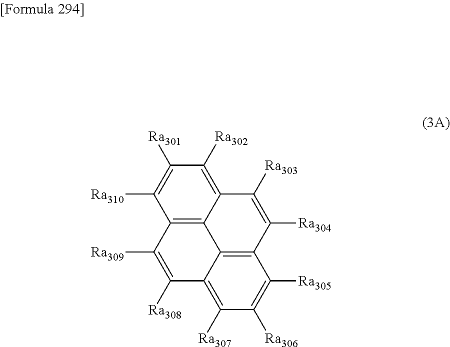

In the formula (3A):
at least one combination of adjacent two or more of Ra₃₀₁, Ra₃₀₂, Ra₃₀₃, Ra₃₀₄, Ra₃₀₅, Ra₃₀₆, Ra₃₀₇, Ra₃₀₈, Ra₃₀₉, and Ra₃₁₀ are mutually bonded to form a substituted or unsubstituted monocyclic ring, mutually bonded to form a substituted or unsubstituted fused ring, or not mutually bonded;
at least one of Ra₃₀₁ to Ra₃₁₀ is a monovalent group represented by a formula (31A) below; and
Ra₃₀₁ to Ra₃₁₀ forming neither the monocyclic ring nor the fused ring and not being the monovalent group represented by the formula (31A) are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

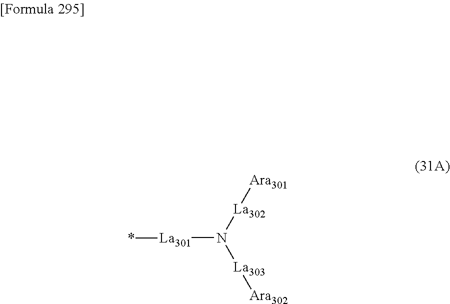

In the formula (31A):
Ara₃₀₁ and Ara₃₀₂ are each independently a substituted or unsubstituted aryl group having 6 to 50 ring carbon atoms, or a substituted or unsubstituted heterocyclic group having 5 to 50 ring atoms;
La₃₀₁, La₃₀₂, and La₃₀₃ are each independently a single bond, a substituted or unsubstituted arylene group having 6 to 30 ring carbon atoms, or a substituted or unsubstituted divalent heterocyclic group having 5 to 30 ring atoms; and
* represents a bonding position to a pyrene ring in the formula (3A).

### Specific Examples of Luminescent Compound

Specific examples of the luminescent compound are given below. It should however be noted that these specific examples are merely exemplary and do not limit the luminescent compound.

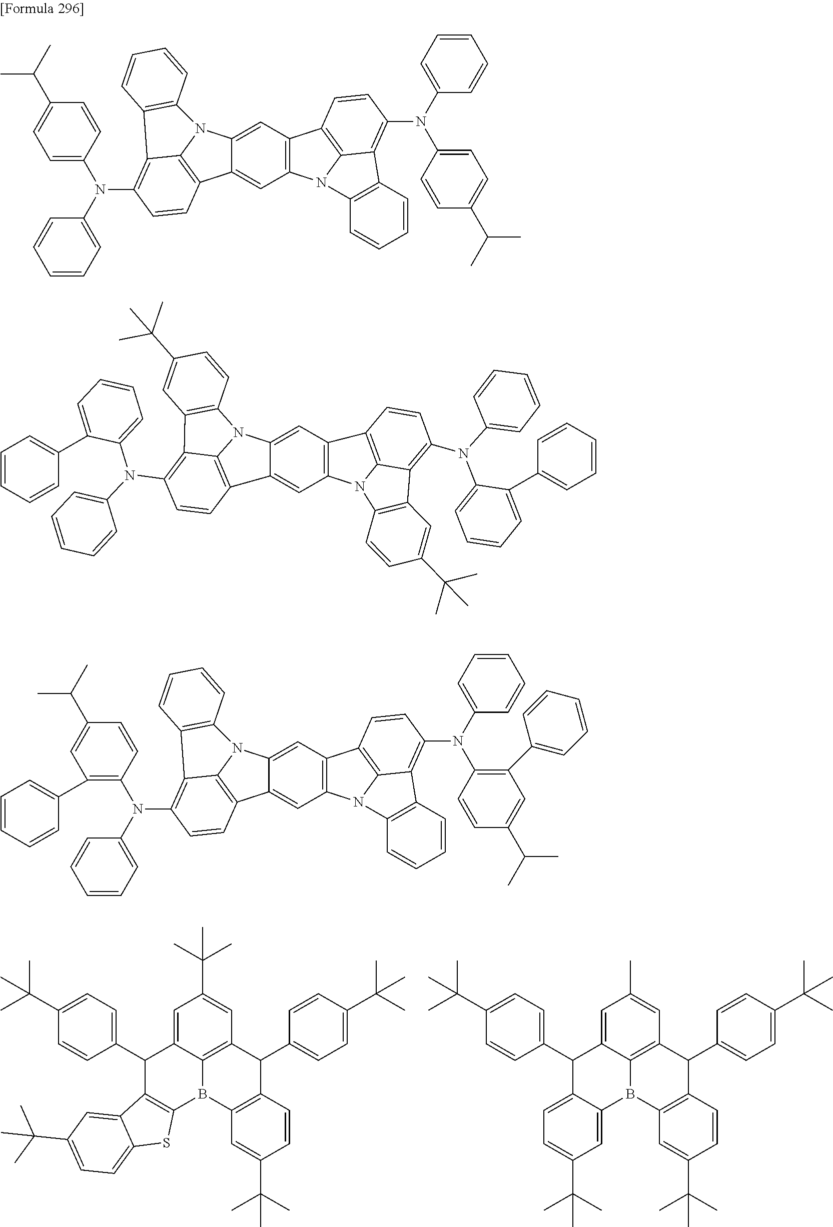

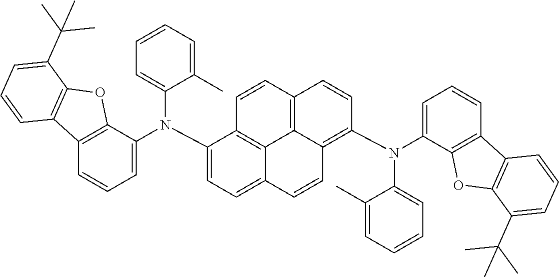 [SMILES: Cc1ccccc1N(c1ccc2ccc3c(N(c4ccccc4C)c4cccc5c4oc4c(C(C)(C)C)cccc45)ccc4ccc1c2c43)c1cccc2c1oc1c(C(C)(C)C)cccc12]

### Additional Layers of Organic EL Device

In addition to the organic layer containing the compound represented by the formula (EB1), the first emitting layer, and the second emitting layer, the organic EL device according to the exemplary embodiment may include one or more organic layers. Examples of the organic layer include at least one layer selected from the group consisting of a hole injecting layer, a hole transporting layer, an electron blocking layer, a hole blocking layer, an electron injecting layer, and an electron transporting layer.

The layers of the organic EL device according to the exemplary embodiment may consist of the organic layer containing the compound represented by the formula (EB1), the first emitting layer, and the second emitting layer, or may further include, for instance, at least one layer selected from the group consisting of a hole injecting layer, a hole transporting layer, an electron blocking layer, a hole blocking layer, an electron injecting layer, and an electron transporting layer.

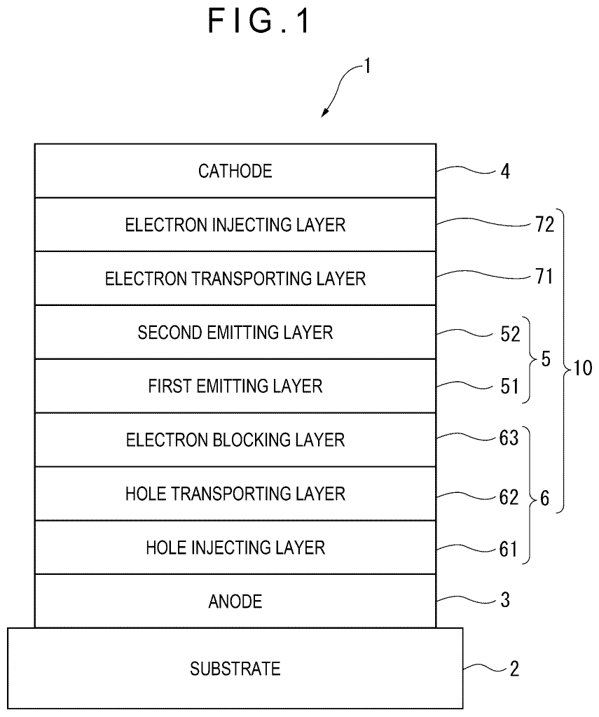

FIG. 1 schematically depicts an exemplary arrangement of the organic EL device according to the exemplary embodiment.

An organic EL device 1 includes a light-transmissive substrate 2, an anode 3, a cathode 4, and organic layers 10 disposed between the anode 3 and the cathode 4. The organic layers 10 of the organic EL device 1 include a hole injecting layer 61, a hole transporting layer 62, an electron blocking layer 63, a first emitting layer 51, a second emitting layer 52, an electron transporting layer 71, and an electron injecting layer 72. In the organic EL device 1, the hole injecting layer 61, the hole transporting layer 62, the electron blocking layer 63, the first emitting layer 51, the second emitting layer 52, the electron transporting layer 71, and the electron injecting layer 72 are layered on the anode 3 in this order. In the organic EL device 1, a hole transporting zone 6 includes the hole injecting layer 61, the hole transporting layer 62, and the electron blocking layer 63, and an emitting zone 5 includes the first emitting layer 51 at a side close to the anode 3 and the second emitting layer 52 at a side close to the cathode 4.

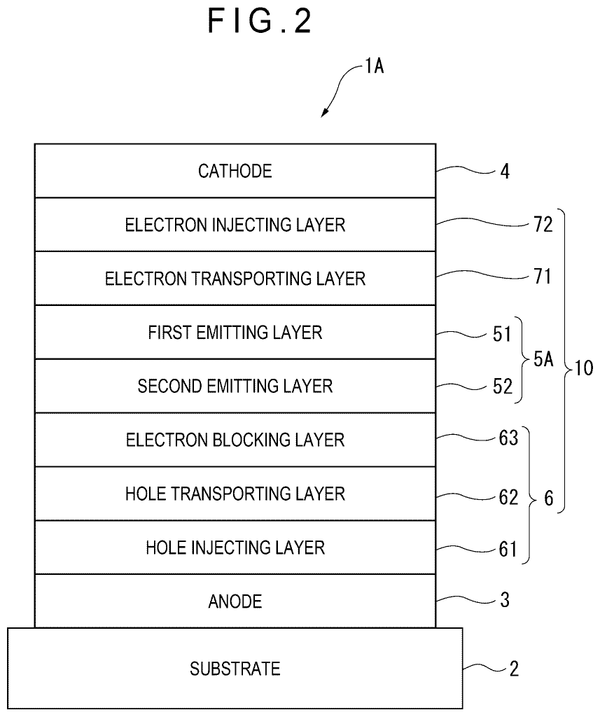

FIG. 2 schematically depicts another exemplary arrangement of the organic EL device according to the exemplary embodiment.

An organic EL device 1A is different from the organic EL device 1 in that an emitting zone 5A includes the second emitting layer 52 at a side close to the anode 3 and the first emitting layer 51 at a side close to the cathode 4, and the rest of components and arrangements of the organic EL device 1A are the same as those of the organic EL device 1. In the organic EL device 1A, the hole injecting layer 61, the hole transporting layer 62, the electron blocking layer 63, the second emitting layer 52, the first emitting layer 51, the electron transporting layer 71, and the electron injecting layer 72 are layered on the anode 3 in this order. In the organic EL device 1A, the hole transporting zone 6 includes the hole injecting layer 61, the hole transporting layer 62, and the electron blocking layer 63.

The invention is not limited to the organic EL device arrangements depicted in FIGS. 1 and 2.

The arrangement of the organic EL device will be further described below. It should be noted that the reference numerals are occasionally omitted below.

### Substrate

The substrate is used as a support for the organic EL device. For instance, glass, quartz, plastics and the like are usable for the substrate. A flexible substrate is also usable. The flexible substrate, which is a bendable substrate, is exemplified by a plastic substrate. Examples of the material for the plastic substrate include polycarbonate, polyarylate, polyethersulfone, polypropylene, polyester, polyvinyl fluoride, polyvinyl chloride, polyimide, and polyethylene naphthalate. Further, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate. Specific examples of the material include indium tin oxide (ITO), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the EL layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to Group 1 or 2 in the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, a mixture thereof, or the like having a small work function (specifically, 3.8 eV or less) for the cathode. Examples of the material for the cathode include elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method, and the like.

### Electron Transporting Layer

In the organic EL device according to the exemplary embodiment, the electron transporting layer is preferably provided between the cathode and the first emitting layer or the second emitting layer disposed close to the cathode.

The electron transporting layer is a layer containing a substance that exhibits a high electron transportability. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinolato)aluminum (abbreviation: Almq₃), bis(10-hydroxybenzo[h]quinolinato)beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the exemplary embodiment, a benzimidazole compound is suitably usable. The above-described substances mostly have an electron mobility of 10⁻⁶ cm²/(V·s) or more. It should be noted that any other substance than the above substances may be used for the electron transporting layer as long as the substance exhibits a higher electron transportability than the hole transportability. The electron transporting layer may be a single layer or a laminate of two or more layers formed of the above substance(s).

Further, a high polymer compound is usable for the electron transporting layer. For instance, poly[(9,9-dihexylfluorene-2,7-diyl)-co-(pyridine-3,5-diyl)](abbreviation: PF-Py), and poly[(9,9-dioctylfluorene-2,7-diyl)-co-(2,2′-bipyridine-6,6′-diyl)](abbreviation: PF-BPy) are usable.

### Specific Examples of Electron Transporting Material

Specific examples of an electron transporting material usable for the electron transporting layer include compounds below. However, the invention is not limited to the specific examples of the electron transporting material.

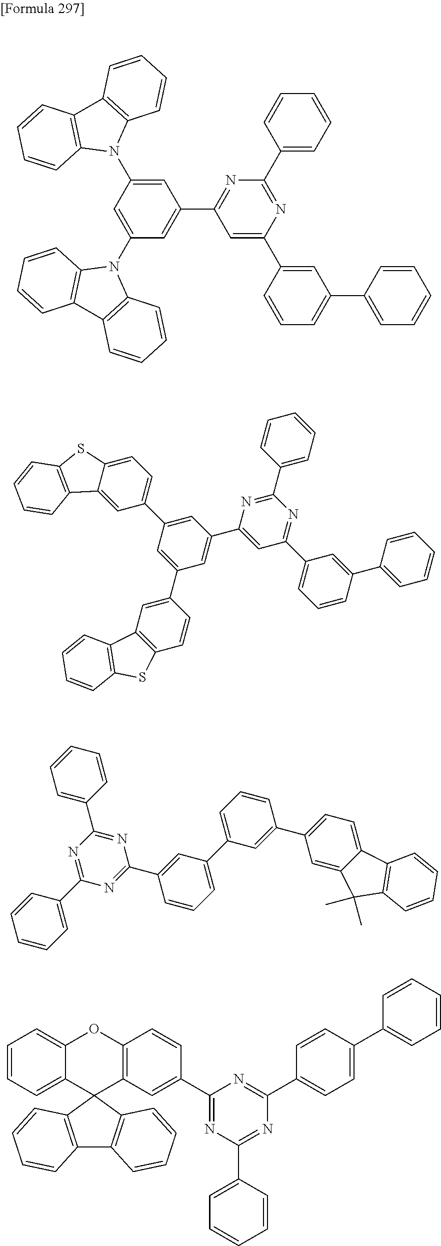 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; c1ccc(-c2cccc(-c3cc(-c4cc(-c5ccc6sc7ccccc7c6c5)cc(-c5ccc6sc7ccccc7c6c5)c4)nc(-c4ccccc4)n3)c2)cc1 ; CC1(C)c2ccccc2-c2ccc(-c3cccc(-c4cccc(-c5nc(-c6ccccc6)nc(-c6ccccc6)n5)c4)c3)cc21 ; c1ccc(-c2ccc(-c3nc(-c4ccccc4)nc(-c4ccc5c(c4)C4(c6ccccc6O5)c5ccccc5-c5ccccc54)n3)cc2)cc1]

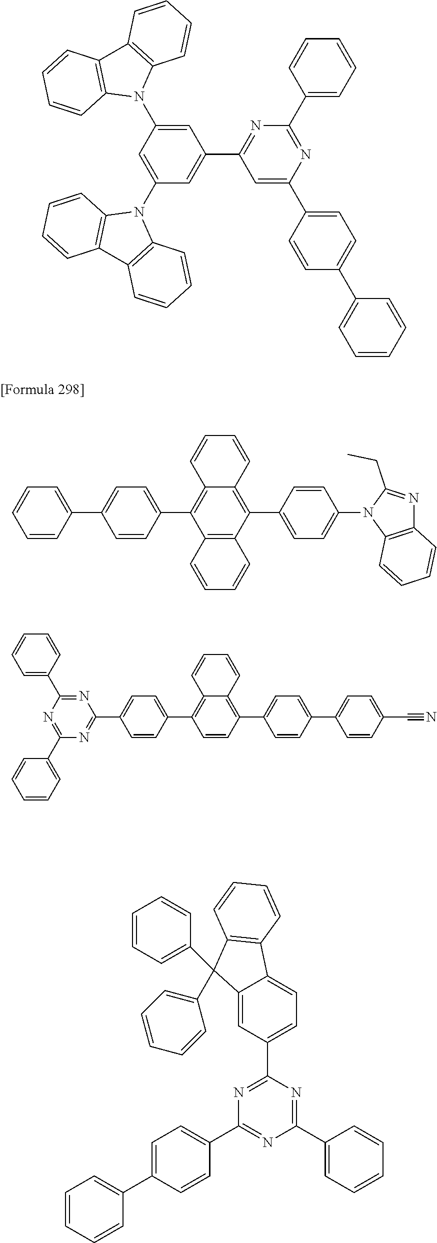 [SMILES: c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; CCc1nc2ccccc2n1-c1ccc(-c2c3ccccc3c(-c3ccc(-c4ccccc4)cc3)c3ccccc23)cc1 ; N#Cc1ccc(-c2ccc(-c3ccc(-c4ccc(-c5nc(-c6ccccc6)nc(-c6ccccc6)n5)cc4)c4ccccc34)cc2)cc1 ; c1ccc(-c2ccc(-c3nc(-c4ccccc4)nc(-c4ccc5c(c4)C(c4ccccc4)(c4ccccc4)c4ccccc4-5)n3)cc2)cc1]

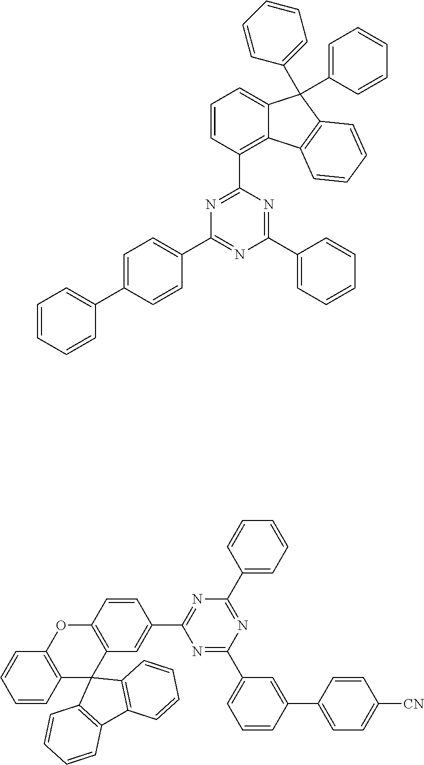 [SMILES: c1ccc(-c2ccc(-c3nc(-c4ccccc4)nc(-c4cccc5c4-c4ccccc4C5(c4ccccc4)c4ccccc4)n3)cc2)cc1 ; N#Cc1ccc(-c2cccc(-c3nc(-c4ccccc4)nc(-c4ccc5c(c4)C4(c6ccccc6O5)c5ccccc5-c5ccccc54)n3)c2)cc1]

### Electron Injecting Layer

The electron injecting layer is a layer that contains a substance exhibiting a high electron injectability. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Layer Formation Method

A method of forming each layer of the organic EL device in the exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

The film thickness of each organic layer of the organic EL device in the exemplary embodiment is not limited unless otherwise specified in the above. In general, the thickness preferably ranges from several nanometers to 1 μm because an excessively small film thickness is likely to cause defects (e.g. pin holes) and an excessively large thickness leads to the necessity of applying high voltage and consequent reduction in efficiency.

### Emission Wavelength of Organic EL Device

The organic electroluminescence device according to the exemplary embodiment preferably emits, when being driven, light whose maximum peak wavelength is 500 nm or less or light whose maximum peak wavelength is in a range from 430 nm to 480 nm.

The maximum peak wavelength of the light emitted from the organic EL device when being driven is measured as follows. Voltage is applied to the organic EL device such that a current density is 10 mA/cm², where spectral radiance spectrum is measured by a spectroradiometer CS-2000 (produced by Konica Minolta Inc.). A peak wavelength of an emission spectrum, at which the luminous intensity of the resultant spectral radiance spectrum is at the maximum, is measured and defined as a maximum peak wavelength (unit: nm).

### Second Exemplary Embodiment

### Electronic Device

An electronic device according to a second exemplary embodiment is installed with the organic EL device according to the above exemplary embodiment. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light. The light-emitting unit can be also used for the display device, for instance, as a backlight of the display device.

### Modifications of Exemplary Embodiments

The scope of the invention is not limited to the above-described exemplary embodiments but includes any modification and improvement as long as such modification and improvement are compatible with the invention.

For instance, the number of emitting layers in the organic EL device is not limited to two, and three or more emitting layers may be provided and layered with each other. When the organic EL device includes two or more emitting layers, it is only necessary that at least two emitting layers (the first emitting layer and second emitting layer) should satisfy the requirements described in the above exemplary embodiment(s). For instance, the rest of the emitting layers may be a fluorescent emitting layer or a phosphorescent emitting layer with use of emission caused by electron transfer from the triplet excited state directly to the ground state.

When the organic EL device includes a plurality of emitting layers, these emitting layers may be mutually adjacently provided, or may form a so-called tandem organic EL device in which a plurality of emitting units are layered via an intermediate layer.

The specific structure, shape, and the like of the components in the invention may be designed in any manner as long as the object of the invention can be achieved.

### EXAMPLES

The invention will be described in further detail with reference to Examples. The scope of the invention is by no means limited to Examples.

### Compounds

Structures of compounds represented by the formula (EB1) and used for producing organic EL devices in Examples 1 to 19 and Comparatives 2 to 4, 7, 8, and 10 are given below.

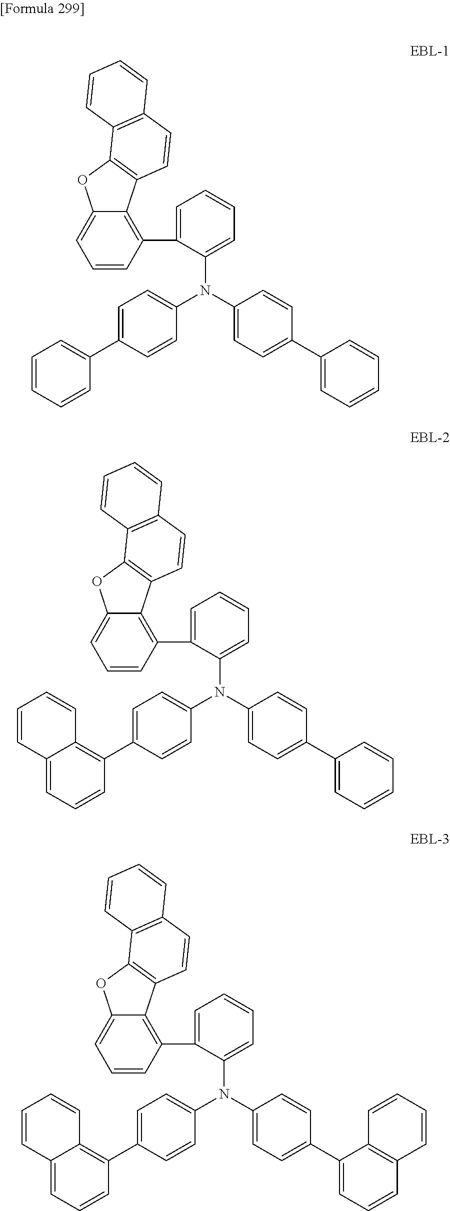 [SMILES: c1ccc(-c2ccc(N(c3ccc(-c4ccccc4)cc3)c3ccccc3-c3cccc4oc5c6ccccc6ccc5c34)cc2)cc1 ; c1ccc(-c2ccc(N(c3ccc(-c4cccc5ccccc45)cc3)c3ccccc3-c3cccc4oc5c6ccccc6ccc5c34)cc2)cc1 ; c1ccc(N(c2ccc(-c3cccc4ccccc34)cc2)c2ccc(-c3cccc4ccccc34)cc2)c(-c2cccc3oc4c5ccccc5ccc4c23)c1]

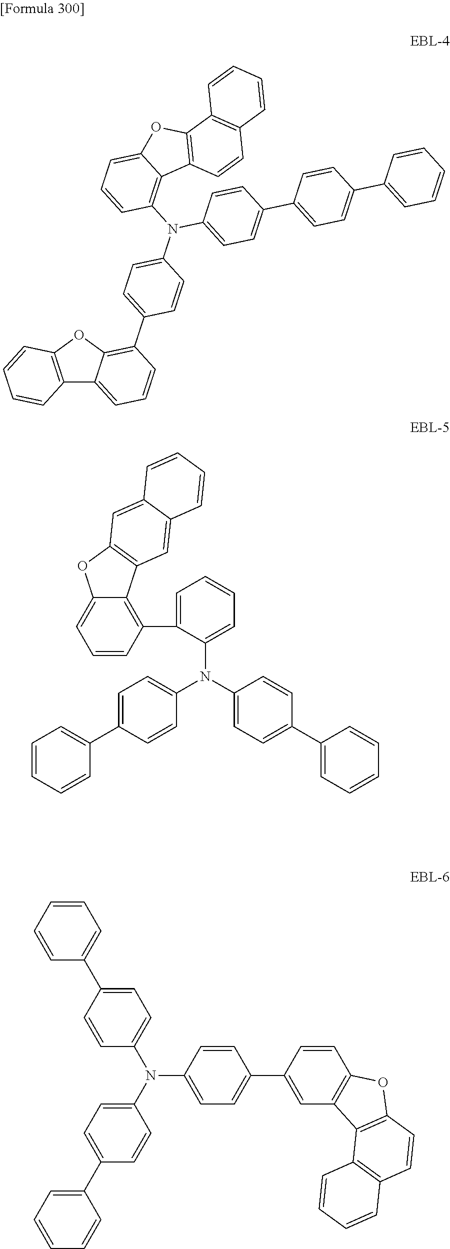 [SMILES: c1ccc(-c2ccc(-c3ccc(N(c4ccc(-c5cccc6c5oc5ccccc56)cc4)c4cccc5oc6c7ccccc7ccc6c45)cc3)cc2)cc1 ; c1ccc(-c2ccc(N(c3ccc(-c4ccccc4)cc3)c3ccccc3-c3cccc4oc5cc6ccccc6cc5c34)cc2)cc1 ; c1ccc(-c2ccc(N(c3ccc(-c4ccccc4)cc3)c3ccc(-c4ccc5oc6ccc7ccccc7c6c5c4)cc3)cc2)cc1]

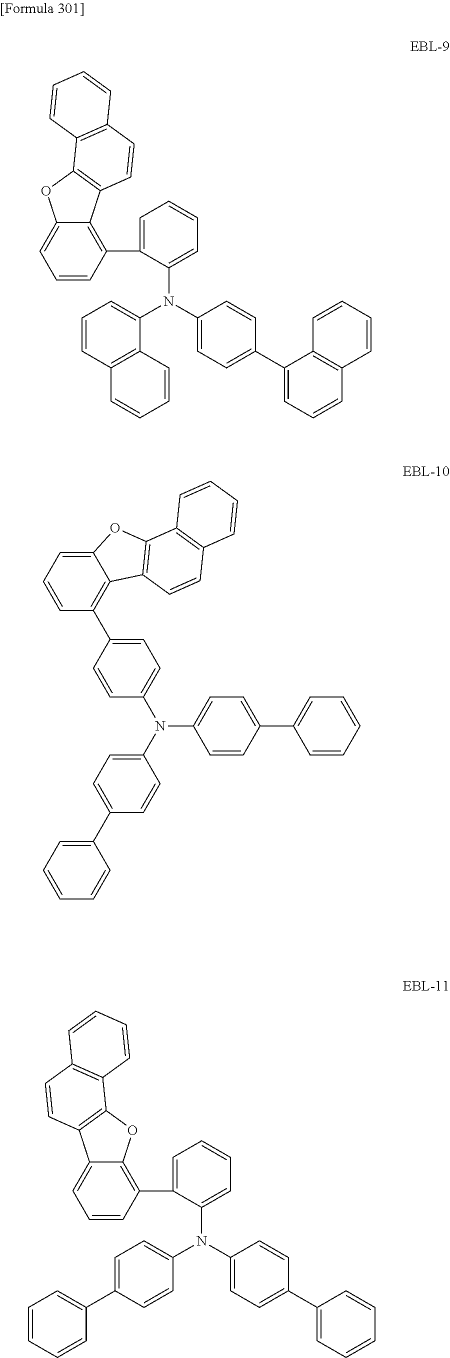 [SMILES: c1ccc(N(c2ccc(-c3cccc4ccccc34)cc2)c2cccc3ccccc23)c(-c2cccc3oc4c5ccccc5ccc4c23)c1 ; c1ccc(-c2ccc(N(c3ccc(-c4ccccc4)cc3)c3ccc(-c4cccc5oc6c7ccccc7ccc6c45)cc3)cc2)cc1 ; c1ccc(-c2ccc(N(c3ccc(-c4ccccc4)cc3)c3ccccc3-c3cccc4c3oc3c5ccccc5ccc43)cc2)cc1]

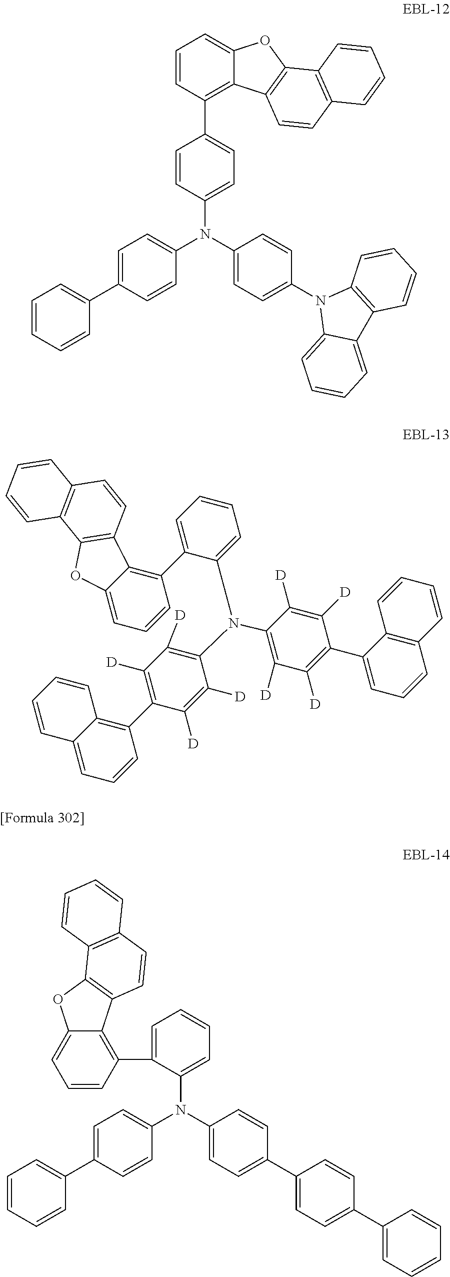 [SMILES: c1ccc(-c2ccc(N(c3ccc(-c4cccc5oc6c7ccccc7ccc6c45)cc3)c3ccc(-n4c5ccccc5c5ccccc54)cc3)cc2)cc1 ; [2H]c1c([2H])c(N(c2ccccc2-c2cccc3oc4c5ccccc5ccc4c23)c2c([2H])c([2H])c(-c3cccc4ccccc34)c([2H])c2[2H])c([2H])c([2H])c1-c1cccc2ccccc12 ; c1ccc(-c2ccc(-c3ccc(N(c4ccc(-c5ccccc5)cc4)c4ccccc4-c4cccc5oc6c7ccccc7ccc6c45)cc3)cc2)cc1]

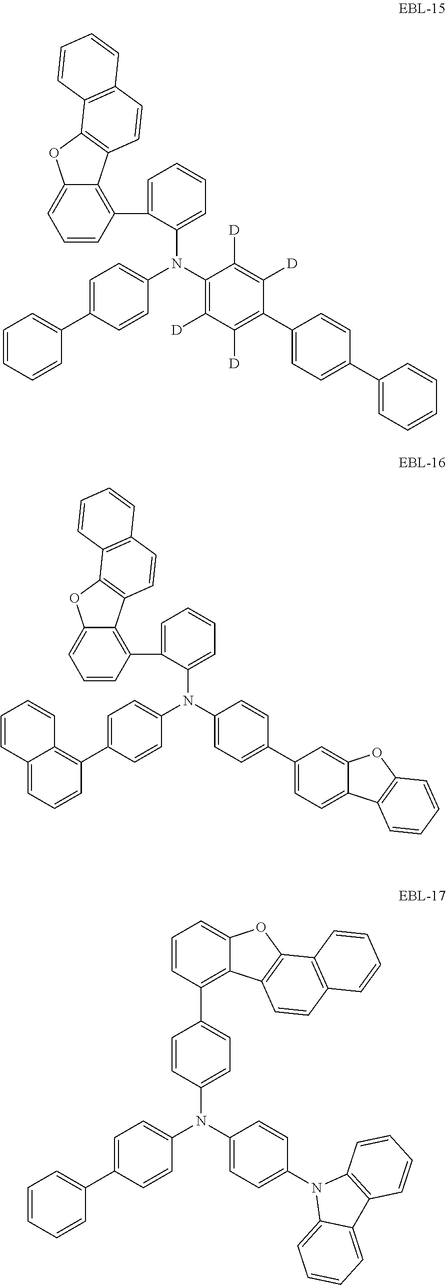 [SMILES: [2H]c1c([2H])c(N(c2ccc(-c3ccccc3)cc2)c2ccccc2-c2cccc3oc4c5ccccc5ccc4c23)c([2H])c([2H])c1-c1ccc(-c2ccccc2)cc1 ; c1ccc(N(c2ccc(-c3ccc4c(c3)oc3ccccc34)cc2)c2ccc(-c3cccc4ccccc34)cc2)c(-c2cccc3oc4c5ccccc5ccc4c23)c1 ; c1ccc(-c2ccc(N(c3ccc(-c4cccc5oc6c7ccccc7ccc6c45)cc3)c3ccc(-n4c5ccccc5c5ccccc54)cc3)cc2)cc1]

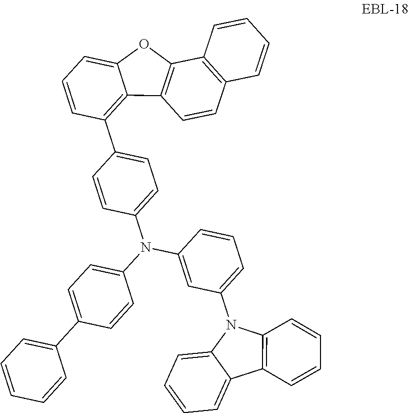 [SMILES: c1ccc(-c2ccc(N(c3ccc(-c4cccc5oc6c7ccccc7ccc6c45)cc3)c3cccc(-n4c5ccccc5c5ccccc54)c3)cc2)cc1]

Structures of comparative compounds used for producing organic EL devices in Comparatives 1, 5, 6, 9, and 11 are given below.

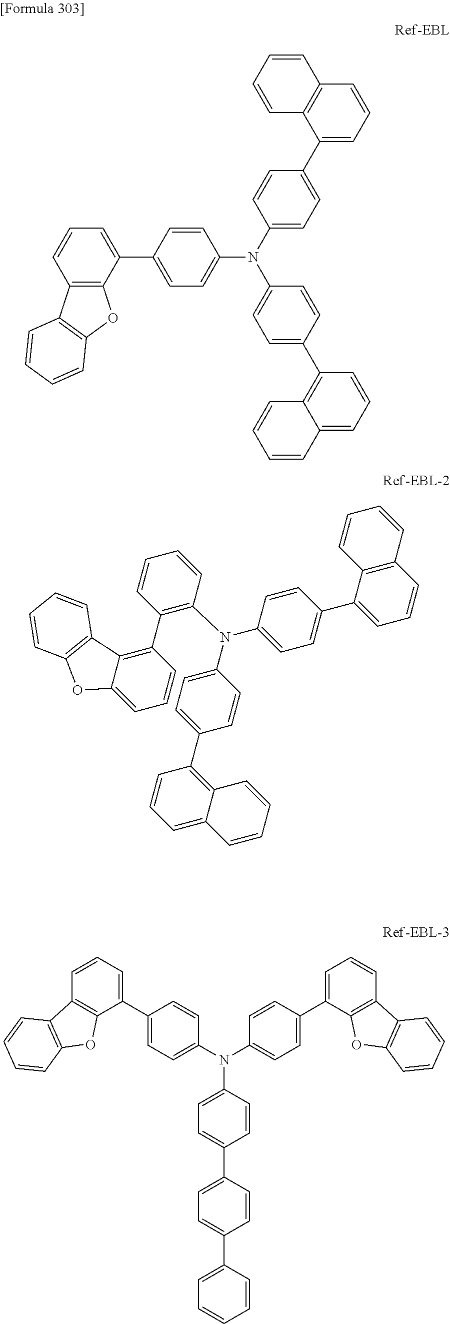 [SMILES: c1ccc2c(-c3ccc(N(c4ccc(-c5cccc6ccccc56)cc4)c4ccc(-c5cccc6c5oc5ccccc56)cc4)cc3)cccc2c1 ; c1ccc(N(c2ccc(-c3cccc4ccccc34)cc2)c2ccc(-c3cccc4ccccc34)cc2)c(-c2cccc3oc4ccccc4c23)c1 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1]

Structures of other compounds used for producing organic EL devices in Examples 1 to 19 and Comparatives 1 to 11 are given below.

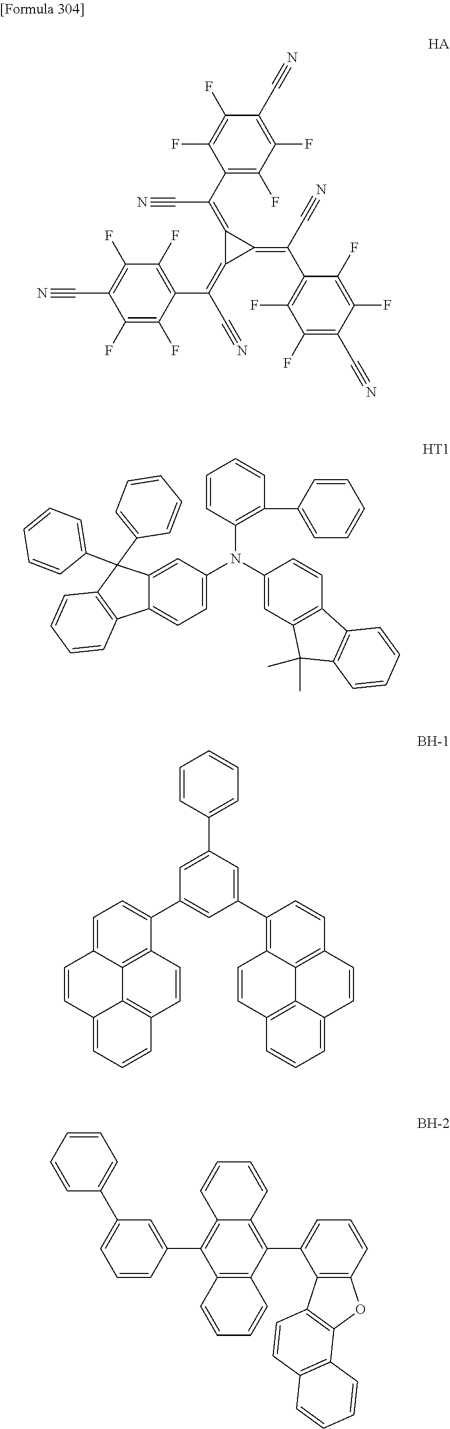

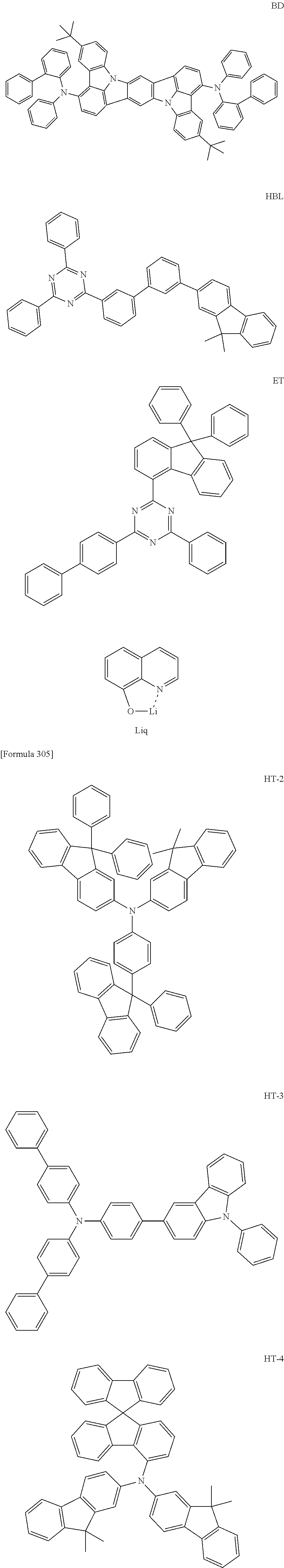

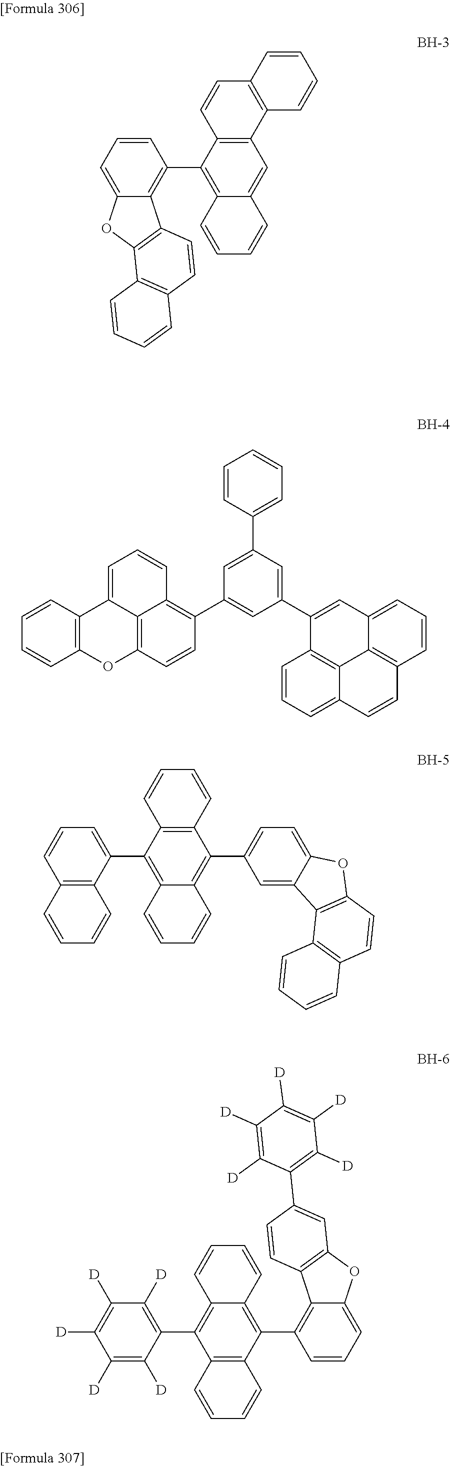 [SMILES: c1ccc2c(-c3cccc4oc5c6ccccc6ccc5c34)c3ccc4ccccc4c3cc2c1 ; c1ccc(-c2cc(-c3ccc4c5c(cccc35)-c3ccccc3O4)cc(-c3cc4cccc5ccc6cccc3c6c54)c2)cc1 ; c1ccc2c(-c3c4ccccc4c(-c4ccc5oc6ccc7ccccc7c6c5c4)c4ccccc34)cccc2c1 ; [2H]c1c([2H])c([2H])c(-c2ccc3c(c2)oc2cccc(-c4c5ccccc5c(-c5c([2H])c([2H])c([2H])c([2H])c5[2H])c5ccccc45)c23)c([2H])c1[2H]]

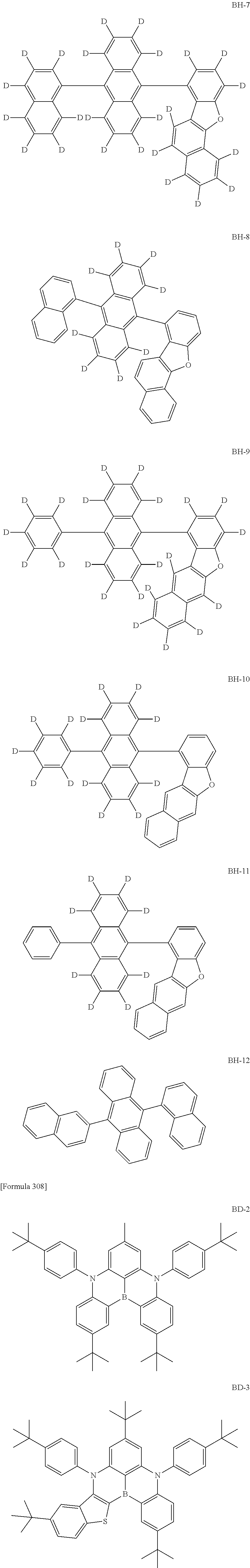

### Production (1) of Organic EL Device

The organic EL devices were produced as follows.

### Example 1

A glass substrate (size: 25 mm×75 mm×1.1 mm thick, produced by Geomatec Co., Ltd.) having an indium tin oxide (ITO) transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum deposition apparatus. First, a compound HT1 and a compound HA were co-deposited on a surface of the glass substrate, where the transparent electrode line was provided, to cover the transparent electrode, thereby forming a 10-nm-thick hole injecting layer. The ratios of the compound HT1 and the compound HA in the hole injecting layer were 97 mass % and 3 mass %, respectively.

The compound HT1 was vapor-deposited on the hole injecting layer to form a 40-nm-thick hole transporting layer.

Subsequently, a compound EBL-1 was vapor-deposited on the hole transporting layer to form a 5-nm-thick electron blocking layer.

A compound BH-1 (first host material) and a compound BD (first luminescent compound) were co-deposited on the electron blocking layer to form a 10-nm-thick first emitting layer. The ratios of the compound BH-1 and the compound BD in the first emitting layer were 99 mass % and 1 mass %, respectively.

A compound BH-2 (second host material) and the compound BD (second luminescent compound) were co-deposited on the first emitting layer, thereby forming a 10-nm-thick second emitting layer. The ratios of the compound BH-2 and the compound BD in the second emitting layer were 99 mass % and 1 mass %, respectively.

A compound HBL was vapor-deposited on the second emitting layer to form a 5-nm-thick first electron transporting layer. The first electron transporting layer is occasionally referred to as a hole blocking layer.

A compound ET and a compound Liq were co-deposited on the first electron transporting layer to form a 25-nm-thick second electron transporting layer. The ratios of the compound ET and the compound Liq in the second electron transporting layer were 50 mass % and 50 mass %, respectively. Liq is an abbreviation of (8-quinolinolato)lithium ((8-Quinolinolato)lithium).

Ytterbium (Yb) was vapor-deposited on the second electron transporting layer to form a 1-nm-thick electron injecting layer.

Metal Al was vapor-deposited on the electron injecting layer to form an 80-nm-thick cathode.

A device arrangement of the organic EL device in Example 1 is roughly shown as follows.

ITO(130)/HT1:HA(10,97%:3%)/HT1(40)/EBL-1(5)/BH-1:BD(10,99%:1%)/BH-2:BD(10,99%:1%)/HBL(5)/ET:Liq(25,50%:50%)/Yb(1)/Al(80)

Numerals in parentheses represent a film thickness (unit: nm).

Regarding the device arrangement of the organic EL device in Example 1, the numerals (97%:3%) represented by percentage in the same parentheses indicate a ratio (mass %) between the compound HT1 and the compound HA in the hole injecting layer, the numerals (99%:1%) represented by percentage in the same parentheses indicate a ratio (mass %) between the host material (compound BH-1 or BH-2) and the luminescent compound (compound BD) in the first emitting layer or the second emitting layer, and the numerals (50%:50%) represented by percentage in the same parentheses indicate a ratio (mass %) between the compound ET and the compound Liq in the second electron transporting layer. Similar notations apply to the description below.

### Examples 2 and 3

The organic EL devices in Examples 2 and 3 were produced as in Example 1 except that the third compound (compound EBL-1) used for forming the electron blocking layer was replaced with the third compound shown in Table 1.

### Comparative 1

The organic EL device in Comparative 1 was produced as in Example 1 except that the third compound (compound EBL-1) used for forming the electron blocking layer was replaced with the third compound shown in Table 1 and that the second emitting layer was formed on the electron blocking layer without forming the first emitting layer.

In Comparative 1, the compound BH-2 (second host material) and the compound BD (second luminescent compound) were co-deposited on the electron blocking layer to form a 20-nm-thick second emitting layer. The ratios of the compound BH-2 and the compound BD in the second emitting layer were 99 mass % and 1 mass %, respectively.

### Comparatives 2 to 4

The organic EL devices in Comparatives 2 to 4 were produced as in Comparative 1 except that the third compound (compound Ref-EBL) used for forming the electron blocking layer was replaced with the third compound shown in Table 1.

### Comparative 5

The organic EL device in Comparative 5 was produced as in Example 1 except that the third compound (compound EBL-1) used for forming the electron blocking layer was replaced with the third compound shown in Table 1.

### Comparative 6

The organic EL device in Comparative 6 was produced as in Example 1 except that the third compound (compound EBL-1) used for forming the electron blocking layer was replaced with the third compound shown in Table 1.

### Production (2) of Organic EL Device

The organic EL devices were produced as follows.

### Example 4

The organic EL device in Example 4 was produced as in Example 1 except that the third compound used for forming the electron blocking layer, the first host material used for forming the first emitting layer, and the second host material used for forming the second emitting layer were respectively replaced with the third compound, the first host material, and the second host material shown in Table 2 and that the film thicknesses of the first and second emitting layers were respectively changed to those shown in Table 2.

A device arrangement of the organic EL device in Example 4 is roughly shown as follows.

ITO(130)/HT1:HA(10,97%:3%)/HT1(40)/EBL-4(5)/BH-3:BD(5,99%:1%)/BH-5:BD(15,99%:1%)/HBL(5)/ET:Liq(25,50%:50%)/Yb(1)/Al(80)

### Comparative 7

The organic EL device in Comparative 7 was produced as in Example 4 except that the second emitting layer was formed on the electron blocking layer without forming the first emitting layer.

In Comparative 7, a compound BH-5 (second host material) and the compound BD (second luminescent compound) were co-deposited on the electron blocking layer to form a 20-nm-thick second emitting layer. The ratios of the compound BH-5 and the compound BD in the second emitting layer were 99 mass % and 1 mass %, respectively.

### Production (3) of Organic EL Device

The organic EL devices were produced as follows.

### Example 5

The organic EL device in Example 5 was produced as in Example 1 except that the third compound used for forming the electron blocking layer, the first host material used for forming the first emitting layer, and the second host material used for forming the second emitting layer were respectively replaced with the third compound, the first host material, and the second host material shown in Table 3 and that the film thicknesses of the first and second emitting layers were respectively changed to those shown in Table 3.

A device arrangement of the organic EL device in Example 5 is roughly shown as follows.

ITO(130)/HT1:HA(10,97%:3%)/HT1(40)/EBL-5(5)/BH-4:BD(5,99%:1%)/BH-5:BD(15,99%:1%)/HBL(5)/ET:Liq(25,50%:50%)/Yb(1)/Al(80)

### Comparative 8

The organic EL device in Comparative 8 was produced as in Example 5 except that the second emitting layer was formed on the electron blocking layer without forming the first emitting layer.

In Comparative 8, the compound BH-5 (second host material) and the compound BD (second luminescent compound) were co-deposited on the electron blocking layer to form a 20-nm-thick second emitting layer. The ratios of the compound BH-5 and the compound BD in the second emitting layer were 99 mass % and 1 mass %, respectively.

### Comparative 9

The organic EL device in Comparative 9 was produced as in Example 5 except that the third compound used for forming the electron blocking layer was replaced with the third compound shown in Table 3.

### Production (4) of Organic EL Device

The organic EL devices were produced as follows.

### Example 6

The organic EL device in Example 6 was produced as in Example 1 except that the third compound used for forming the electron blocking layer, the first host material used for forming the first emitting layer, and the second host material used for forming the second emitting layer were respectively replaced with the third compound, the first host material, and the second host material shown in Table 4 and that the film thicknesses of the first and second emitting layers were respectively changed to those shown in Table 4.

A device arrangement of the organic EL device in Example 6 is roughly shown as follows.

ITO(130)/HT1:HA(10,97%:3%)/HT1(40)/EBL-6(5)/BH-3:BD(5,99%:1%)/BH-6:BD(15,99%:1%)/HBL(5)/ET:Liq(25,50%:50%)/Yb(1)/Al(80)

### Comparative 10

The organic EL device in Comparative 10 was produced as in Example 6 except that the second emitting layer was formed on the electron blocking layer without forming the first emitting layer.

In Comparative 10, a compound BH-6 (second host material) and the compound BD (second luminescent compound) were co-deposited on the electron blocking layer to form a 20-nm-thick second emitting layer. The ratios of the compound BH-6 and the compound BD in the second emitting layer were 99 mass % and 1 mass %, respectively.

### Production (5) of Organic EL Device

The organic EL devices were produced as follows.

### Example 7

A glass substrate (size: 25 mm×75 mm×1.1 mm thick, produced by Geomatec Co., Ltd.) having an indium tin oxide (ITO) transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum deposition apparatus. First, the compound HT1 and the compound HA were co-deposited on a surface of the glass substrate, where the transparent electrode line was provided, to cover the transparent electrode, thereby forming a 10-nm-thick hole injecting layer. The ratios of the compound HT1 and the compound HA in the hole injecting layer were 97 mass % and 3 mass %, respectively.

A compound HT-2 was vapor-deposited on the hole injecting layer to form a 40-nm-thick hole transporting layer.

Subsequently, a compound EBL-9 was vapor-deposited on the hole transporting layer to form a 5-nm-thick electron blocking layer.

The compound BH-1 (first host material) and a compound BD-2 (first luminescent compound) were co-deposited on the electron blocking layer to form a 10-nm-thick first emitting layer. The ratios of the compound BH-1 and the compound BD-2 in the first emitting layer were 99 mass % and 1 mass %, respectively.

The compound BH-2 (second host material) and the compound BD-2 (second luminescent compound) were co-deposited on the first emitting layer, thereby forming a 10-nm-thick second emitting layer. The ratios of the compound BH-2 and the compound BD-2 in the second emitting layer were 99 mass % and 1 mass % respectively.

The compound HBL was vapor-deposited on the second emitting layer to form a 5-nm-thick first electron transporting layer. The first electron transporting layer is occasionally referred to as a hole blocking layer.

The compound ET and the compound Liq were co-deposited on the first electron transporting layer to form a 25-nm-thick second electron transporting layer. The ratios of the compound ET and the compound Liq in the second electron transporting layer were 50 mass % and 50 mass %, respectively.

Ytterbium (Yb) was vapor-deposited on the second electron transporting layer to form a 1-nm-thick electron injecting layer.

Metal Al was vapor-deposited on the electron injecting layer to form an 80-nm-thick cathode.

A device arrangement of the organic EL device in Example 7 is roughly shown as follows.

ITO(130)/HT1:HA(10,97%:3%)/HT-2(40)/EBL-9(5)/BH-1:BD-2(10,99%:1%)/BH-2:BD-2(10,99%:1%)/HBL(5)/ET:Liq(25,50%:50%)/Yb(1)/Al(80)

### Examples 8 to 11

The organic EL devices in Example 8 to 11 were each produced as in Example 1 except that at least one of the hole transporting zone material (compound HT1) used for forming the hole transporting layer, the third compound (compound EBL-1) used for forming the electron blocking layer, the first host material (compound BH-1) used for forming the first emitting layer, the first luminescent compound (BD) used for forming the first emitting layer, the second host material (compound BH-2) used for forming the second emitting layer, or the second luminescent compound (BD) used for forming the second emitting layer was changed to the hole transporting zone material, the third compound, the first host material, the first luminescent compound, the second host material, or the second luminescent compound shown in Table 5.

Comparatives 1 to 6 are shown again in Table 5 for comparison.

### Production (6) of Organic EL Device

The organic EL devices were produced as follows.

### Example 12

A glass substrate (size: 25 mm×75 mm×1.1 mm thick, produced by Geomatec Co., Ltd.) having an indium tin oxide (ITO) transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum deposition apparatus. First, a compound HT-3 and the compound HA were co-deposited on a surface of the glass substrate, where the transparent electrode line was provided, to cover the transparent electrode, thereby forming a 10-nm-thick hole injecting layer. The ratios of the compound HT-3 and the compound HA in the hole injecting layer were 97 mass % and 3 mass %, respectively.

The compound HT-3 was vapor-deposited on the hole injecting layer to form an 80-nm-thick hole transporting layer.

Subsequently, a compound EBL-14 was vapor-deposited on the hole transporting layer to form a 10-nm-thick electron blocking layer.

The compound BH-1 (first host material) and a compound BD-3 (first luminescent compound) were co-deposited on the electron blocking layer to form a 5-nm-thick first emitting layer. The ratios of the compound BH-1 and the compound BD-3 in the first emitting layer were 99 mass % and 1 mass %, respectively.

A compound BH-7 (second host material) and the compound BD-3 (second luminescent compound) were co-deposited on the first emitting layer, thereby forming a 15-nm-thick second emitting layer. The ratios of the compound BH-7 and the compound BD-3 in the second emitting layer were 99 mass % and 1 mass %, respectively.

The compound HBL was vapor-deposited on the second emitting layer to form a 5-nm-thick first electron transporting layer. The first electron transporting layer is occasionally referred to as a hole blocking layer.

The compound ET and the compound Liq were co-deposited on the first electron transporting layer to form a 25-nm-thick second electron transporting layer. The ratios of the compound ET and the compound Liq in the second electron transporting layer were 50 mass % and 50 mass %, respectively.

Ytterbium (Yb) was vapor-deposited on the second electron transporting layer to form a 1-nm-thick electron injecting layer.

Metal Al was vapor-deposited on the electron injecting layer to form an 80-nm-thick cathode.

A device arrangement of the organic EL device in Example 12 is roughly shown as follows.

ITO(130)/HT-3:HA(10,97%:3%)/HT-3(80)/EBL-14(10)/BH-1:BD-3(5,99%:1%)/BH-7:BD-3(15,99%:1%)/HBL(5)/ET:Liq(25,50%:50%)/Yb(1)/Al(80)

### Examples 13 to 15 and Example 18

The organic EL devices in Examples 13 to 15 and Example 18 were each produced as in Example 12 except that at least one of the hole transporting zone material (compound HT-3) used for forming the hole transporting layer, the third compound (compound EBL-14) used for forming the electron blocking layer, the first luminescent compound (BD-3), the second host material (compound BH-2) used for forming the second emitting layer, or the second luminescent compound (BD) used for forming the second emitting layer was changed to the hole transporting zone material, the third compound, the first luminescent compound, the second host material, or the second luminescent compound shown in Table 6.

### Examples 16, 17 and 19

The organic EL devices in Examples 16, 17 and 19 were each produced as in Example 12 except that at least one of the hole transporting zone material (compound HT-3) used for forming the hole transporting layer, the third compound (compound EBL-14) used for forming the electron blocking layer, the first luminescent compound (BD-3), the second host material (compound BH-2) used for forming the second emitting layer, or the second luminescent compound (BD-3) used for forming the second emitting layer was changed to the hole transporting zone material, the third compound, the first luminescent compound, the second host material, or the second luminescent compound shown in Table 6. In the organic EL device in each of Examples 16, 17 and 19, the second emitting layer was formed by co-depositing two types of the second host material and the second luminescent compound.

In Example 16, the mass ratio between a compound BH-9 and a compound BH-12 was 50:50.

In Example 17, the mass ratio between a compound BH-10 and the compound BH-12 was 40:60.

In Example 19, the mass ratio between a compound BH-11 and the compound BH-12 was 50:50.

In the Examples 16, 17 and 19, the ratio of the total of the two types of the second host material in the second emitting layer was 99 mass % and the ratio of the second luminescent compound in the second emitting layer was 1 mass %.

### Comparative 11

The organic EL device in Comparative 11 was produced as in Example 12 except that the second emitting layer was formed on the electron blocking layer without forming the first emitting layer, the third compound (compound EBL-14) used for forming the electron blocking layer and the second luminescent compound (BD-3) used for forming the second emitting layer were respectively replaced with the third compound and the second luminescent compound shown in Table 6.

### Evaluation on Organic EL Devices

The produced organic EL devices were evaluated as follows. Tables 1 to 6 show the evaluation results.

### External Quantum Efficiency EQE

Voltage was applied to the organic EL device produced in each of Examples 1 to 19 and Comparatives 1 to 11 such that a current density was 10 mA/cm², where spectral radiance spectrum was measured by a spectroradiometer CS-2000 (produced by Konica Minolta, Inc.). The external quantum efficiency EQE was calculated based on the obtained spectral radiance spectra, assuming that the spectra was provided under a Lambertian radiation. The unit for EQE is denoted by %.

Table 1 shows the relative value of EQE calculated based on EQE in Examples 1 to 3 and Comparatives 1 to 6 according to a numerical formula (Numerical Formula X1) below. The unit for the relative value of EQE is denoted by %.

$$EQE ( relative value ) = ( EQE of each Example / EQE of Comparative 1 ) \times 100 \qquad ( Numerical Formula X 1 )$$

Table 2 shows the relative value of EQE calculated based on EQE in Example 4 and Comparative 7 according to a numerical formula (Numerical Formula X11) below.

$$EQE ( relative value ) = ( EQE of each Example / EQE of Comparative 7 ) \times 100 \qquad ( Numerical Formula X 11 )$$

Table 3 shows the relative value of EQE calculated based on EQE in Example 5 and Comparatives 8 and 9 according to a numerical formula (Numerical Formula X12) below.

$$EQE ( relative value ) = ( EQE of each Example / EQE of Comparative 8 ) \times 100 \qquad ( Numerical Formula X 12 )$$

Table 4 shows the relative value of EQE calculated based on EQE in Example 6 and Comparative 10 according to a numerical formula (Numerical Formula X13) below.

$$EQE ( relative value ) = ( EQE of each Example / EQE of Comparative 10 ) \times 100 \qquad ( Numerical Formula X 13 )$$

Table 5 shows the relative value of EQE calculated based on EQE in Examples 7 to 11 and Comparatives 1 to 6 according to a numerical formula (Numerical Formula X14) below.

$$EQE ( relative value ) = ( EQE of each Example / EQE of Comparative 1 ) \times 100 \qquad ( Numerical Formula X 14 )$$

Table 6 shows the relative value of EQE calculated based on EQE in Examples 12 to 19 and Comparative 11 according to a numerical formula (Numerical Formula X15) below.

$$EQE ( relative value ) = ( EQE of each Example / EQE of Comparative 11 ) \times 100 \qquad ( Numerical Formula X15 )$$

### Lifetime LT95

Voltage was applied to the organic EL device produced in each of Examples 1 to 19 and Comparatives 1 to 11 so that a current density was 50 mA/cm², where a time (LT95 (unit: hr)) elapsed before a luminance intensity was reduced to 95% of the initial luminance intensity was measured as the lifetime. The luminance intensity was measured by using a spectroradiometer CS-2000 (produced by Konica Minolta, Inc.).

Table 1 shows the relative value of LT95 calculated based on LT95 in Examples 1 to 3 and Comparatives 1 to 6 according to a numerical formula (Numerical Formula X2) below. The unit for the relative value of LT95 is denoted by %.

$$LT 95 ( relative value ) = ( LT 95 of each Example / LT 95 of Comparative 1 ) \times 100 \qquad ( Numerical Formula X2 )$$

Table 2 shows the relative value of LT95 calculated based on LT95 in Example 4 and Comparative 7 according to a numerical formula (Numerical Formula X21) below.

$$LT 95 ( relative value ) = ( LT 95 of each Example / LT 95 of Comparative 7 ) \times 100 \qquad ( Numerical Formula X21 )$$

Table 3 shows the relative value of LT95 calculated based on LT95 in Example 5 and Comparatives 8 and 9 according to a numerical formula (Numerical Formula X22) below.

$$LT 95 ( relative value ) = ( LT 95 of each Example / LT 95 of Comparative 8 ) \times 100 \qquad ( Numerical Formula X22 )$$

Table 4 shows the relative value of LT95 calculated based on LT95 in Example 6 and Comparative 10 according to a numerical formula (Numerical Formula X23) below.

$$LT 95 ( relative value ) = ( LT 95 of each Example / LT 95 of Comparative 10 ) \times 100 \qquad ( Numerical Formula X23 )$$

Table 5 shows the relative value of LT95 calculated based on LT95 in Example 7 to 11 and Comparatives 1 to 6 according to a numerical formula (Numerical Formula X24) below.

$$LT 95 ( relative value ) = ( LT 95 of each Example / LT 95 of Comparative 1 ) \times 100 \qquad ( Numerical Formula X24 )$$

Table 6 shows the relative value of LT95 calculated based on LT95 in Example 12 to 19 and Comparative 11 according to a numerical formula (Numerical Formula X25) below.

$$LT 95 ( relative value ) = ( LT 95 of each Example / LT 95 of Comparative 11 ) \times 100 \qquad ( Numerical Formula X25 )$$

TABLE 1

| | | Emitting zone | | | | | | | | | |
|---|---|---|---|---|---|---|---|---|---|---|---|
| | Electron | First emitting layer | | | | Second emitting layer | | | | | |
| | blocking layer | First host | | First | | Second host | | Second | | Device evaluation | |
| | Third | material | | luminescent | Film | material | | luminescent | Film | EQE | LT95 |
| | compound | | T₁ | compound | thickness | | T₁ | compound | thickness | (Relative | (Relative |
| | Name | Name | [eV] | Name | [nm] | Name | [eV] | Name | [nm] | value) | value) |
| Ex. 1 | EBL-1 | BH-1 | 2.1 | BD | 10 | BH-2 | 1.9 | BD | 10 | 109% | 307% |
| Ex. 2 | EBL-2 | BH-1 | 2.1 | BD | 10 | BH-2 | 1.9 | BD | 10 | 109% | 305% |
| Ex. 3 | EBL-3 | BH-1 | 2.1 | BD | 10 | BH-2 | 1.9 | BD | 10 | 110% | 282% |
| Comp. 1 | Ref-EBL | — | — | — | — | BH-2 | 1.9 | BD | 20 | 100% | 100% |
| Comp. 2 | EBL-1 | — | — | — | — | BH-2 | 1.9 | BD | 20 | 101% | 122% |
| Comp. 3 | EBL-2 | — | — | — | — | BH-2 | 1.9 | BD | 20 | 101% | 120% |
| Comp. 4 | EBL-3 | — | — | — | — | BH-2 | 1.9 | BD | 20 | 102% | 112% |
| Comp. 5 | Ref-EBL | BH-1 | 2.1 | BD | 10 | BH-2 | 1.9 | BD | 10 | 106% | 141% |
| Comp. 6 | Ref-EBL-2 | BH-1 | 2.1 | BD | 10 | BH-2 | 1.9 | BD | 10 | 108% | 98% |

In the organic EL device in each of Examples 1 to 3, the electron blocking layer in the hole transporting zone contained a compound represented by the formula (EB1) and the first emitting layer and the second emitting layer in the emitting zone contained host materials satisfying the numerical formula (Numerical Formula 1A). As a result, the organic EL devices in Examples 1 to 3 emitted light with higher efficiency and had a longer lifetime than the organic EL devices in Comparatives 1 to 6. As for the organic EL device in each of Comparatives 2 to 4, although the electron blocking layer contained a compound represented by the formula (EB1), the emitting zone only included the second emitting layer without including the first emitting layer. The effect of improving the luminous efficiency and extending the lifetime in Comparatives 2 to 4 was thus lower than that in Examples 1 to 3. Accordingly, the organic EL device achieved the improved luminous efficiency and longer lifetime by disposing a layer that contained a compound represented by the formula (EB1) on the anode side with respect to the emitting zone including layered emitting layers.

TABLE 2

| | | Emitting zone | | | | | | | | | |
|---|---|---|---|---|---|---|---|---|---|---|---|
| | Electron | First emitting layer | | | | Second emitting layer | | | | | |
| | blocking layer | First host | | First | | Second host | | Second | | Device evaluation | |
| | Third | material | | luminescent | Film | material | | luminescent | Film | EQE | LT95 |
| | compound | | T₁ | compound | thickness | | T₁ | compound | thickness | (Relative | (Relative |
| | Name | Name | [eV] | Name | [nm] | Name | [eV] | Name | [nm] | value) | value) |
| Ex. 4 | EBL-4 | BH-3 | 2.1 | BD | 5 | BH-5 | 1.8 | BD | 15 | 112% | 227% |
| Comp. 7 | EBL-4 | — | — | — | — | BH-5 | 1.8 | BD | 20 | 100% | 100% |

In the organic EL device in Example 4, the electron blocking layer in the hole transporting zone contained a compound represented by the formula (EB1) and the first emitting layer and the second emitting layer in the emitting zone contained host materials satisfying the numerical formula (Numerical Formula 1A). As a result, the organic EL device in Example 4 emitted light with higher efficiency and had a longer lifetime than the organic EL device in Comparative 7. As for the organic EL device in Comparative 7, although the electron blocking layer contained a compound represented by the formula (EB1), the emitting zone only included the second emitting layer without including the first emitting layer. The effect of improving the luminous efficiency and extending the lifetime in Comparative 7 was thus lower than that in Example 4. Accordingly, the organic EL device achieved the improved luminous efficiency and longer lifetime by disposing a layer that contained a compound represented by the formula (EB1) on the anode side with respect to the emitting zone including layered emitting layers.

TABLE 3

| | | Emitting zone | | | | | | | | | |
|---|---|---|---|---|---|---|---|---|---|---|---|
| | Electron | First emitting layer | | | | Second emitting layer | | | | | |
| | blocking layer | First host | | First | | Second host | | Second | | Device evaluation | |
| | Third | material | | luminescent | Film | material | | luminescent | Film | EQE | LT95 |
| | compound | | T₁ | compound | thickness | | T₁ | compound | thickness | (Relative | (Relative |
| | Name | Name | [eV] | Name | [nm] | Name | [eV] | Name | [nm] | value) | value) |
| Ex. 5 | EBL-5 | BH-4 | 2.1 | BD | 5 | BH-5 | 1.8 | BD | 15 | 110% | 258% |
| Comp. 8 | EBL-5 | — | — | — | — | BH-5 | 1.8 | BD | 20 | 100% | 100% |
| Comp. 9 | Ref-EBL-3 | BH-4 | 2.1 | BD | 5 | BH-5 | 1.8 | BD | 15 | 103% | 100% |

In the organic EL device in Example 5, the electron blocking layer in the hole transporting zone contained a compound represented by the formula (EB1) and the first emitting layer and the second emitting layer in the emitting zone contained host materials satisfying the numerical formula (Numerical Formula 1A). As a result, the organic EL device in Example 5 emitted light with higher efficiency and had a longer lifetime than the organic EL devices in Comparatives 8 and 9. As for the organic EL device in Comparative 8, although the electron blocking layer contained a compound represented by the formula (EB1), the emitting zone only included the second emitting layer without including the first emitting layer. The effect of improving the luminous efficiency and extending the lifetime in Comparative 8 was thus lower than that in Example 5. Accordingly, the organic EL device achieved the improved luminous efficiency and longer lifetime by disposing a layer that contained a compound represented by the formula (EB1) on the anode side with respect to the emitting zone including layered emitting layers.

TABLE 4

| | | Emitting zone | | | | | | | | | |
|---|---|---|---|---|---|---|---|---|---|---|---|
| | Electron | First emitting layer | | | | Second emitting layer | | | | | |
| | blocking layer | First host | | First | | Second host | | Second | | Device evaluation | |
| | Third | material | | luminescent | Film | material | | luminescent | Film | EQE | LT95 |
| | compound | | T₁ | compound | thickness | | T₁ | compound | thickness | (Relative | (Relative |
| | Name | Name | [eV] | Name | [nm] | Name | [eV] | Name | [nm] | value) | value) |
| Ex. 6 | EBL-6 | BH-3 | 2.1 | BD | 5 | BH-6 | 1.8 | BD | 15 | 104% | 243% |
| Comp. 10 | EBL-6 | — | — | — | — | BH-6 | 1.8 | BD | 20 | 100% | 100% |

In the organic EL device in Example 6, the electron blocking layer in the hole transporting zone contained a compound represented by the formula (EB1) and the first emitting layer and the second emitting layer in the emitting zone contained host materials satisfying the numerical formula (Numerical Formula 1A). As a result, the organic EL device in Example 6 emitted light with higher efficiency and had a longer lifetime than the organic EL device in Comparative 10. As for the organic EL device in Comparative 10, although the electron blocking layer contained a compound represented by the formula (EB1), the emitting zone only included the second emitting layer without including the first emitting layer. The effect of improving the luminous efficiency and extending the lifetime in Comparative 10 was thus lower than that in Example 6. Accordingly, the organic EL device achieved the improved luminous efficiency and longer lifetime by disposing a layer that contained a compound represented by the formula (EB1) on the anode side with respect to the emitting zone including layered emitting layers.

TABLE 5

| | Hole | | | | | | | | | | | |
|---|---|---|---|---|---|---|---|---|---|---|---|---|
| | transporting | | | | | | | | | | | |
| | layer | Electron | Emitting zone | | | | | | | | | |
| | Hole | blocking | First emitting layer | | | | Second emitting layer | | | | | |
| | transporting | layer | First host | | First | Film | Second host | | Second | Film | Device evaluation | |
| | zone | Third | material | | luminescent | thick- | material | | luminescent | thick- | EQE | LT95 |
| | material | compound | | T₁ | compound | ness | | T₁ | compound | ness | (Relative | (Relative |
| | Name | Name | Name | [eV] | Name | [nm] | Name | [eV] | Name | [nm] | value) | value) |
| Ex. 7 | HT-2 | EBL-9 | BH-1 | 2.1 | BD-2 | 10 | BH-2 | 1.9 | BD-2 | 10 | 114% | 266% |
| Ex. 8 | HT-3 | EBL-10 | BH-1 | 2.1 | BD-3 | 10 | BH-2 | 1.9 | BD-3 | 10 | 111% | 274% |
| Ex. 9 | HT-1 | EBL-11 | BH-1 | 2.1 | BD-2 | 10 | BH-2 | 1.9 | BD-2 | 10 | 110% | 310% |
| Ex. 10 | HT-4 | EBL-12 | BH-1 | 2.1 | BD-3 | 10 | BH-2 | 1.9 | BD-3 | 10 | 113% | 270% |
| Ex. 11 | HT-1 | EBL-13 | BH-1 | 2.1 | BD-3 | 10 | BH-2 | 1.9 | BD-3 | 10 | 111% | 302% |
| Comp. 1 | HT-1 | Ref-EBL | — | — | — | — | BH-2 | 1.9 | BD | 20 | 100% | 100% |
| (reshown) | | | | | | | | | | | | |
| Comp. 2 | HT-1 | EBL-1 | — | — | — | — | BH-2 | 1.9 | BD | 20 | 101% | 122% |
| (reshown) | | | | | | | | | | | | |
| Comp. 3 | HT-1 | EBL-2 | — | — | — | — | BH-2 | 1.9 | BD | 20 | 101% | 120% |
| (reshown) | | | | | | | | | | | | |
| Comp. 4 | HT-1 | EBL-3 | — | — | — | — | BH-2 | 1.9 | BD | 20 | 102% | 112% |
| (reshown) | | | | | | | | | | | | |
| Comp. 5 | HT-1 | Ref-EBL | BH-1 | 2.1 | BD | 10 | BH-2 | 1.9 | BD | 10 | 106% | 141% |
| (reshown) | | | | | | | | | | | | |
| Comp. 6 | HT-1 | Ref-EBL-2 | BH-1 | 2.1 | BD | 10 | BH-2 | 1.9 | BD | 10 | 108% | 98% |
| (reshown) | | | | | | | | | | | | |

In the organic EL device in each of Examples 7 to 11, the electron blocking layer in the hole transporting zone contained a compound represented by the formula (EB1) and the first emitting layer and the second emitting layer in the emitting zone contained host materials satisfying the numerical formula (Numerical Formula 1A). As a result, the organic EL devices in Examples 7 to 11 emitted light with higher efficiency and had a longer lifetime than the organic EL devices in Comparatives 1 to 6. As for the organic EL device in each of Comparatives 2 to 4, although the electron blocking layer contained a compound represented by the formula (EB1), the emitting zone only included the second emitting layer without including the first emitting layer. The effect of improving the luminous efficiency and extending the lifetime in Comparatives 2 to 4 was thus lower than that in Examples 7 to 11. Accordingly, the organic EL device achieved the improved luminous efficiency and longer lifetime by disposing a layer that contained a compound represented by the formula (EB1) on the anode side with respect to the emitting zone including layered emitting layers.

TABLE 6

| | Hole | | | | | | | | | | | |
|---|---|---|---|---|---|---|---|---|---|---|---|---|
| | transporting | | | | | | | | | | | |
| | layer | Electron | Emitting zone | | | | | | | | | |
| | Hole | blocking | First emitting layer | | | | Second emitting layer | | | | | |
| | transporting | layer | First host | | First | Film | Second host | | Second | Film | Device evaluation | |
| | zone | Third | material | | luminescent | thick- | material | | luminescent | thick- | EQE | LT95 |
| | material | compound | | T₁ | compound | ness | | T₁ | compound | ness | (Relative | (Relative |
| | Name | Name | Name | [eV] | Name | [nm] | Name | [eV] | Name | [nm] | value) | value) |
| Ex. 12 | HT-3 | EBL-14 | BH-1 | 2.1 | BD-3 | 5 | BH-7 | 1.8 | BD-3 | 15 | 106% | 183% |
| Ex. 13 | HT-3 | EBL-15 | BH-1 | 2.1 | BD-3 | 5 | BH-8 | 1.8 | BD-3 | 15 | 105% | 201% |
| Ex. 14 | HT-3 | EBL-16 | BH-1 | 2.1 | BD-3 | 5 | BH-7 | 1.8 | BD-3 | 15 | 107% | 195% |
| Ex. 15 | HT-4 | EBL-17 | BH-1 | 2.1 | BD-2 | 5 | BH-7 | 1.8 | BD-2 | 15 | 108% | 192% |
| Ex. 16 | HT-4 | EBL-17 | BH-1 | 2.1 | BD-2 | 5 | BH-9:BH-12 | 1.9 | BD-2 | 15 | 104% | 221% |
| | | | | | | | (50:50) | | | | | |
| Ex. 17 | HT-4 | EBL-17 | BH-1 | 2.1 | BD-2 | 5 | BH-10:BH-12 | 1.9 | BD-2 | 15 | 105% | 208% |
| | | | | | | | (40:60) | | | | | |
| Ex. 18 | HT-4 | EBL-18 | BH-1 | 2.1 | BD-2 | 5 | BH-8 | 1.8 | BD-2 | 15 | 106% | 178% |
| Ex. 19 | HT-4 | EBL-18 | BH-1 | 2.1 | BD-2 | 5 | BH-11:BH-12 | 1.9 | BD-2 | 15 | 107% | 190% |
| | | | | | | | (50:50) | | | | | |
| Comp. 11 | HT-3 | Ref-EBL-3 | — | — | — | — | BH-7 | 1.8 | BD | 20 | 100% | 100% |

In the organic EL device in each of Examples 12 to 19, the electron blocking layer in the hole transporting zone contained a compound represented by the formula (EB1) and the first emitting layer and the second emitting layer in the emitting zone contained host materials satisfying the numerical formula (Numerical Formula 1A). As a result, the organic EL devices in Examples 12 to 19 emitted light with higher efficiency and had a longer lifetime than the organic EL device in Comparative 11. Accordingly, the organic EL device achieved the improved luminous efficiency and longer lifetime by disposing a layer that contained a compound represented by the formula (EB1) on the anode side with respect to the emitting zone including layered emitting layers.

### Evaluation on Compounds

The following evaluation was conducted on the compounds used for producing the organic EL devices. Table 7 shows the evaluation results.

### Triplet Energy T₁

A measurement target compound was dissolved in EPA (diethylether:isopentane:ethanol=5:5:2 in volume ratio) at a concentration of 10 μmol/L, and the obtained solution was put in a quartz cell to provide a measurement sample. A phosphorescence spectrum (ordinate axis: phosphorescent luminous intensity, abscissa axis: wavelength) of the measurement sample was measured at a low temperature (77K). A tangent was drawn to the rise of the phosphorescence spectrum close to the short-wavelength region. An energy amount was calculated by a conversion equation (F1) below on a basis of a wavelength value λ_{edge} [nm] at an intersection of the tangent and the abscissa axis. The calculated energy amount was defined as triplet energy T₁. It should be noted that the triplet energy T₁ may have an error of about plus or minus 0.02 eV depending on measurement conditions.

$$Conversion Equation ( F 1 ) : {T}_{1} [ eV ] = 1239.85 / {λ}_{edge}$$

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength region of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 produced by Hitachi High-Technologies Corporation was used.

### Singlet Energy S₁

A toluene solution of a measurement target compound at a concentration of 10 μmol/L was prepared and put in a quartz cell. An absorption spectrum (ordinate axis: absorption intensity, abscissa axis: wavelength) of the thus-obtained sample was measured at a normal temperature (300K). A tangent was drawn to the fall of the absorption spectrum close to the long-wavelength region, and a wavelength value λedge [nm] at an intersection of the tangent and the abscissa axis was assigned to a conversion equation (F2) below to calculate a singlet energy.

$$Conversion Equation ( F 2 ) : {S}_{1} [ eV ] = 1239.85 / λ edge$$

A spectrophotometer (U3310 produced by Hitachi, Ltd.) was used for measuring the absorption spectrum.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

### Measurement of Maximum Fluorescence Peak Wavelength (FL-Peak) and Full Width at Half Maximum

A measurement target compound was dissolved in toluene at a concentration of 4.9×10⁻⁶ mol/L to prepare a toluene solution. Using a fluorescence spectrometer (spectrophotofluorometer F-7000 manufactured by Hitachi High-Tech Science Corporation), the toluene solution of the measurement target compound was excited at 390 nm, where a maximum fluorescence peak wavelength A (unit: nm) was measured. The full width at half maximum FWHM (unit: nm) at the maximum peak obtained based on the measured fluorescence spectrum was determined as a full width at half maximum of the measurement target compound. FWHM is an abbreviation of the full width at half maximum.

TABLE 7

| | S₁ | T₁ | λ | FWHM |
|---|---|---|---|---|
| Compound | [eV] | [eV] | [nm] | [nm] |
| BH-1 | 3.3 | 2.1 | — | — |
| BH-2 | 3.0 | 1.9 | — | — |
| BH-3 | 3.1 | 2.1 | — | — |
| BH-4 | 3.0 | 2.1 | — | — |
| BH-5 | 3.0 | 1.8 | — | — |
| BH-6 | 3.0 | 1.8 | — | — |
| BH-7 | 3.0 | 1.8 | — | — |
| BH-8 | 3.0 | 1.8 | — | — |
| BH-9 | 3.0 | 1.8 | — | — |
| BH-10 | 3.0 | 1.8 | — | — |
| BH-11 | 3.0 | 1.8 | — | — |
| BH-12 | 3.0 | 1.9 | — | — |
| BD | 2.8 | 2.3 | 432 | 17 |
| BD-2 | 2.7 | 2.6 | 455 | 23 |
| BD-3 | 2.8 | 2.5 | 457 | 22 |

In the organic EL device in each of Examples 16, 17, and 19, a plurality of types of the second host material were mixed in the second emitting layer, and the value of the triplet energy T₁ of the second host material shown in Table 6 was a value calculated based on the triplet energy T₁ of each second host material contained in the second emitting layer and a mixture ratio thereof. For instance, in Example 16, the second emitting layer contained two types of the second host material, the compounds BH-9 and BH-12, at a mass ratio of 50:50. The value of the triplet energy T₁ of the second host material was calculated as follows.

$$1. 8 \times ( 50 / 100 ) + 1.9 \times ( 50 / 100 ) = 1.85 1.9$$

### SYNTHESIS EXAMPLES

### Synthesis Example 1: Synthesis of Compound EBL-5

### Synthesis of Intermediate M1

An intermediate M1 was synthesized through a synthesis pathway below.

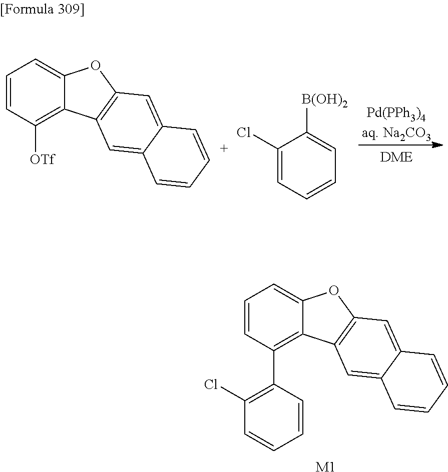

Under argon atmosphere, a mixture of 11.0 g (30.0 mmol) of benzo[b]naphtho[2,3-d]furan-1-yl-trifluoromethanesulfonate, 5.16 g (33.0 mmol) of 2-chlorophenylboronic acid, 1.04 g (0.90 mmol) of tetrakis(triphenylphosphine)palladium(0), 37.5 mL (75.0 mmol) of 2M sodium carbonate aqueous solution, and 100 mL of DME was refluxed at the boiling point for five hours. After being refluxed, the reaction solution was cooled to room temperature, which was then concentrated under reduced pressure. The obtained residue was purified by silica gel column chromatography to obtain 8.15 g of a white solid. The yield was 83%. DME is an abbreviation for 1,2-dimethoxyethane.

Synthesis of Compound EBL-5 The compound EBL-5 was synthesized through a synthesis pathway below.

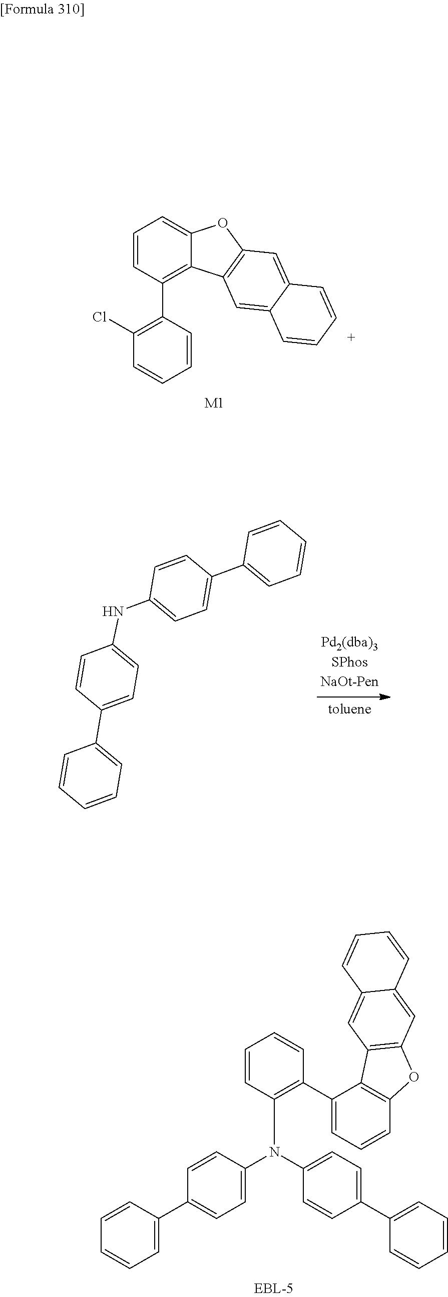

Under argon atmosphere, a mixture of 3.45 g (10.5 mmol) of the intermediate M1, 3.21 g (21.0 mmol) of N-[1,1′-biphenyl]-4-yl[1,1′-biphenyl]-4-amine, 0.366 g (0.400 mmol) of tris(dibenzylideneacetone)dipalladium(0), 0.328 g (0.800 mmol) of 2-dicyclohexylphosphino-2′,6′-dimethoxybiphenyl (SPhos), 4.2 mL of sodium t-pentoxide (40% toluene solution), and 67 mL of toluene was refluxed at the boiling point for seven hours. After being refluxed, the reaction solution was cooled to room temperature, which was then concentrated under reduced pressure. The obtained residue was purified by silica gel column chromatography and recrystallization to obtain 4.16 g of a white solid. The yield was 68%. As a result of mass spectroscopy analysis, the obtained white solid was the compound EBL-5, and had m/e=614 while a molecular weight was 613.76.

### Synthesis Example 2: Synthesis of Compound BH-3

A compound BH-3 was synthesized through a synthesis pathway below.

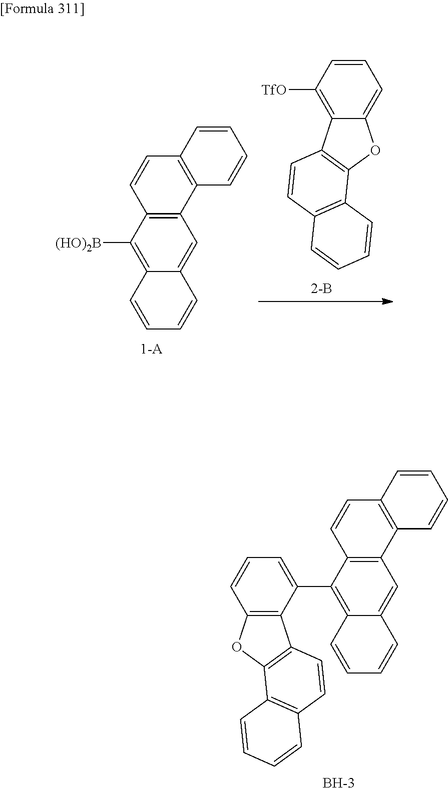

Under argon atmosphere, 17.6 g (64.5 mmol) of an intermediate 1-A, 23.6 g (64.5 mmol) of an intermediate 2-B, 0.91 g (1.29 mmol) of dichlorobisamphospalladium(II), 90.0 ml (180.0 mmol) of 2M sodium carbonate aqueous solution, and 450 ml of 1,2-dimethoxyethane were put into a flask and heated to reflux with stirring at 100 degrees C. for eight hours. After stirring, the reaction solution was cooled to a room temperature (25 degrees C.) and the solvent was distilled off. The obtained solid was purified by silica-gel column chromatography to obtain 24.1 g (a yield of 84%) of a light yellow solid.

The light yellow solid was identified as the compound BH-3 by analysis according to liquid chromatography-mass spectrometry (LC-MS).

EXPLANATION OF CODES

1 . . . organic EL device, 1A . . . organic EL device, 10 . . . organic layer,
2 . . . substrate, 3 . . . anode, 4 . . . cathode, 5 . . . emitting zone, 51 . . . first emitting layer,
52 . . . second emitting layer, 5A . . . emitting zone, 6 . . . hole transporting zone, 61 . . . hole
injecting layer, 62 . . . hole transporting layer, 63 . . . electron blocking layer, 71 . . . electron
transporting layer, 72 . . . electron injecting layer.

1: An organic electroluminescence device, comprising:
an anode;
a cathode;
an emitting zone disposed between the anode and the cathode; and
a hole transporting zone disposed between the anode and the emitting zone, wherein
the emitting zone comprises a first emitting layer and a second emitting layer,
the first emitting layer comprises a first host material and a first luminescent compound that emits light having a maximum peak wavelength of 500 nm or less,
the second emitting layer comprises a second host material and a second luminescent compound that emits light having a maximum peak wavelength of 500 nm or less,
the first host material and the second host material are mutually different,
a triplet energy of the first host material T₁(H1) and a triplet energy of the second host material T₁(H2) satisfy Numerical Formula 1A below:
$${T}_{1} ( H 1 ) > {T}_{1} ( H 2 ) \qquad ( Numerical Formula 1 A )$$
the first luminescent compound and the second luminescent compound are mutually the same or different, and
the hole transporting zone comprises one or more organic layers, and at least one of the organic layers comprises a third compound represented by a formula (EB1) below: 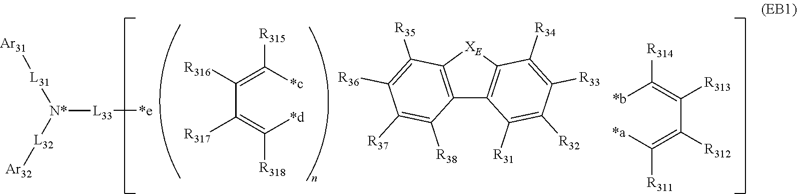
where, in the formula (EB1):
N* is a central nitrogen atom;
R₃₁ to R₃₈ and R₃₁₁ to R₃₁₈ are each independently a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms;
R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different;
when two or more R₉₀₇ are present, the two or more R₉₀₇ are mutually the same or different;
n is 0 or 1;
when n is 0, one of R₃₁ and R₃₂, one of R₃₂ and R₃₃, or one of R₃₃ and R₃₄ is a single bond with *a, and the other of R₃₁ and R₃₂, the other of R₃₂ and R₃₃, or the other of R₃₃ and R₃₄ is a single bond with *b; and one selected from R₃₁ to R₃₄ being neither the single bond with *a nor the single bond with *b, R₃₅ to R₃₈, and R₃₁₁ to R₃₁₄ is a single bond with *e;
when n is 1, one of R₃₁ and R₃₂, one of R₃₂ and R₃₃, or one of R₃₃ and R₃₄ is a single bond with *a, and the other of R₃₁ and R₃₂, the other of R₃₂ and R₃₃, or the other of R₃₃ and R₃₄ is a single bond with *b; one of R₃₅ and R₃₆, one of R₃₆ and R₃₇, or one of R₃₇ and R₃₈ is a single bond with *c, and the other of R₃₅ and R₃₆, the other of R₃₆ and R₃₇, or the other of R₃₇ and R₃₈ is a single bond with *d; and one selected from R₃₁ to R₃₄ being neither the single bond with *a nor the single bond with *b, R₃₅ to R₃₈ being neither the single bond with *c nor the single bond with *d, R₃₁₁ to R₃₁₄, and R₃₁₅ to R₃₁₈ is a single bond with *e;
X_{E} is an oxygen atom or a sulfur atom;
Ar₃₁ and Ar₃₂ are each independently a substituted or unsubstituted aryl group having 6 to 30 ring carbon atoms, or a substituted or unsubstituted heterocyclic group having 5 to 30 ring atoms; and
L₃₁ to L₃₃ are each independently a single bond, a substituted or unsubstituted arylene group having 6 to 30 ring carbon atoms, or a substituted or unsubstituted divalent heterocyclic group having 5 to 30 ring atoms.

2: The organic electroluminescence device according to claim 1, wherein the hole transporting zone comprises two or more organic layers, and at least one of the organic layers comprises the third compound represented by the formula (EB1).

3: The organic electroluminescence device according to claim 1, wherein the hole transporting zone comprises three or more organic layers, and at least one of the organic layers comprises the third compound represented by the formula (EB1).

4: The organic electroluminescence device according to claim 1, wherein one of the organic layers included in the hole transporting zone and disposed closest to the cathode comprises the third compound represented by the formula (EB1).

5: The organic electroluminescence device according to claim 1, wherein R₃₈ is a single bond with *e.

6: The organic electroluminescence device according to claim 1, wherein the third compound represented by the formula (EB1) is a compound represented by any of formulae (EB11) to (EB13) below, 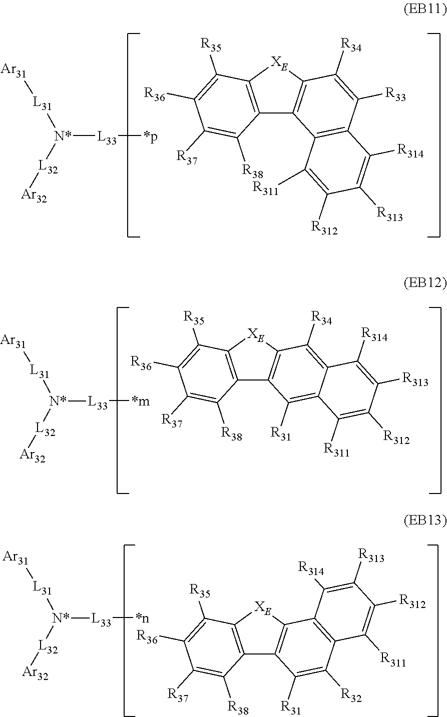
where, in the formulae (EB11) to (EB13):
N*, R₃₁ to R₃₈, R₃₁₁ to R₃₁₄, X_{E}, Ar₃₁, Ar₃₂, and L₃₁ to L₃₃ respectively represent the same as those defined in the formula (EB1);
when the compound represented by the formula (EB1) is a compound represented by the formula (EB11), one selected from R₃₃ to R₃₈ and R₃₁₁ to R₃₁₄ is a single bond with *p;
when the compound represented by the formula (EB1) is a compound represented by the formula (EB12), one selected from R₃₁, R₃₄ to R₃₈, and R₃₁₁ to R₃₁₄ is a single bond with *m; and
when the compound represented by the formula (EB1) is a compound represented by the formula (EB13), one selected from R₃₁, R₃₂, R₃₅ to R₃₈, and R₃₁₁ to R₃₁₄ is a single bond with *n.

7: The organic electroluminescence device according to claim 6, wherein the compound represented by the formula (EB1) is a compound represented by the formula (EB13).

8: The organic electroluminescence device according to claim 6, wherein R₃₈ in the formula (EB11), (EB12), or (EB13) is a single bond with *p, *m, or *n.

9: The organic electroluminescence device according to claim 1, wherein L₃₃ is a substituted or unsubstituted phenylene group.

10: The organic electroluminescence device according to claim 1, wherein L₃₃ is an unsubstituted phenylene group.

11: The organic electroluminescence device according to claim 1, wherein L₃₃ is a substituted or unsubstituted o-phenylene group or a substituted or unsubstituted p-phenylene group.

12: The organic electroluminescence device according to claim 1, wherein the compound represented by the formula (EB1) is a compound represented by a formula (EB131) below, 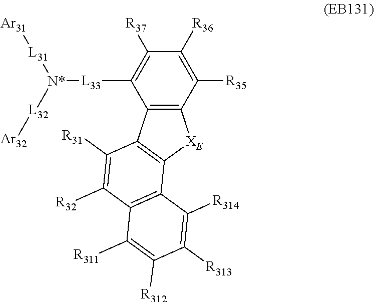
where, in the formula (EB131), N*, R₃₁, R₃₂, R₃₅ to R₃₇, R₃₁₁ to R₃₁₄, X_{E}, Ar₃₁, Ar₃₂, and L₃₁ to L₃₃ respectively represent the same as those defined in the formula (EB1).

13: The organic electroluminescence device according to claim 1, wherein X_{E} is an oxygen atom.

14: The organic electroluminescence device according to claim 1, wherein Ar₃₁ and Ar₃₂ in the formula (EB1) are each independently a group represented by any of formulae (1-a) to (1-f) below;
when Ar₃₁ is a group represented by any of the formulae (1-a) to (1-f), L₃₁ is a single bond or an unsubstituted arylene group having 6 to 30 ring carbon atoms; and
when Ar₃₂ is a group represented by any of the formulae (1-a) to (1-f), L₃₂ is a single bond or an unsubstituted arylene group having 6 to 30 ring carbon atoms, 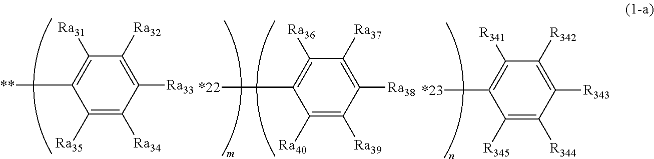
where, in the formula (1-a):
R₃₄₁ to R₃₄₅ are each independently a hydrogen atom, a substituted or unsubstituted alkyl group having 1 to 10 carbon atoms, or a substituted or unsubstituted aryl group having 6 to 12 ring carbon atoms;
adjacent two selected from R₃₄₁ to R₃₄₅ are not mutually bonded and thus form no ring;
one selected from Ra₃₁ to Ra₃₅ is a single bond with *22;
one selected from Ra₃₆ to Ra₄₀ is a single bond with *23;
Ra₃₁ to Ra₄₀ not being the single bond are each independently a hydrogen atom, an unsubstituted alkyl group having 1 to 10 carbon atoms, or an unsubstituted aryl group having 6 to 12 ring carbon atoms;
adjacent two selected from Ra₃₁ to Ra₃₅ not being the single bond are not mutually bonded and thus form no ring;
adjacent two selected from Ra₃₆ to Ra₄₀ not being the single bond are not mutually bonded and thus form no ring;
** represents a bonding position to L₃₁ or L₃₂;
m is 0 or 1, and n is 0 or 1;
when m and n are each 0, *23 represents a bonding position to L₃₁ or L₃₂;
when m is 0 and n is 1, *22 represents a bonding position to L₃₁ or L₃₂;
when m is 1 and n is 0, one selected from Ra₃₁ to Ra₃₅ is a single bond with *23;
when L₃₁ is a single bond, **, *22, or *23 of the group represented by the formula (1-a), which is Ar₃₁, represents a bonding position to the central nitrogen atom N*; and
when L₃₂ is a single bond, **, *22, or *23 of the group represented by the formula (1-a), which is Ar₃₂, represents a bonding position to the central nitrogen atom N*, 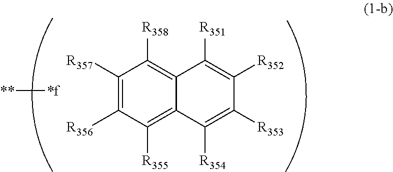
where, in the formula (1-b):
R₃₅₁ to R₃₅₈ are each independently a hydrogen atom, a substituted or unsubstituted alkyl group having 1 to 6 carbon atoms, or a substituted or unsubstituted aryl group having 6 to 12 ring carbon atoms;
one selected from R₃₅₁ to R₃₅₈ is a single bond with *f;
adjacent two selected from R₃₅₁ to R₃₅₈ not being the single bond are not mutually bonded and thus form no cyclic structure;
** represents a bonding position to L₃₁ or L₃₂;
when L₃₁ is a single bond, ** of the group represented by the formula (1-b), which is Ar₃₁, represents a bonding position to the central nitrogen atom N*; and
when L₃₂ is a single bond, ** of the group represented by the formula (1-b), which is Ar₃₂, represents a bonding position to the central nitrogen atom N*, 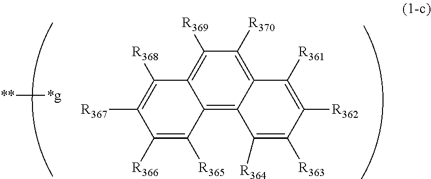
where, in the formula (1-c):
R₃₆₁ to R₃₇₀ are each independently a hydrogen atom, a substituted or unsubstituted alkyl group having 1 to 6 carbon atoms, or a substituted or unsubstituted aryl group having 6 to 12 ring carbon atoms;
one selected from R₃₆₁ to R₃₇₀ is a single bond with *g;
adjacent two selected from R₃₆₁ to R₃₇₀ not being the single bond are not mutually bonded and thus form no cyclic structure;
** represents a bonding position to L₃₁ or L₃₂;
when L₃₁ is a single bond, ** of the group represented by the formula (1-c), which is Ar₃₁, represents a bonding position to the central nitrogen atom N*; and
when L₃₂ is a single bond, ** of the group represented by the formula (1-c), which is Ar₃₂, represents a bonding position to the central nitrogen atom N*, 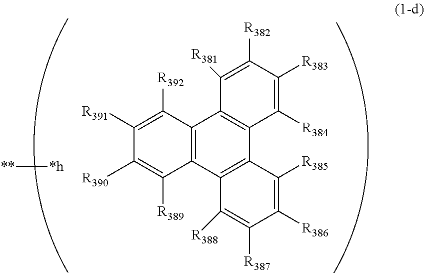
where, in the formula (1-d):
R₃₈₁ to R₃₉₂ are each independently a hydrogen atom, a substituted or unsubstituted alkyl group having 1 to 6 carbon atoms, or a substituted or unsubstituted aryl group having 6 to 12 ring carbon atoms;
one selected from R₃₈₁ to R₃₉₂ is a single bond with *h;
adjacent two selected from R₃₈₁ to R₃₉₂ not being the single bond are not mutually bonded and thus form no cyclic structure;
** represents a bonding position to L₃₁ or L₃₂;
when L₃₁ is a single bond, ** of the group represented by the formula (1-d), which is Ar₃₁, represents a bonding position to the central nitrogen atom N*; and
when L₃₂ is a single bond, ** of the group represented by the formula (1-d), which is Ar₃₂, represents a bonding position to the central nitrogen atom N*, 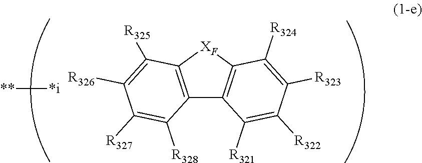
where, in the formula (1-e):
R₃₂₁ to R₃₂₈ are each independently a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
X_{F} is an oxygen atom, a sulfur atom, NR_{F1}, or CR_{F2}R_{F3};
R_{F1} is a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
R_{F2} and R_{F3} are each independently a hydrogen atom, a substituted or unsubstituted alkyl group having 1 to 50 carbon atoms, or a substituted or unsubstituted aryl group having 6 to 50 ring carbon atoms, and R_{F2} and R_{F3} are mutually bonded to form a substituted or unsubstituted monocyclic ring, mutually bonded to form a substituted or unsubstituted fused ring, or not mutually bonded;
one selected from R₃₂₁ to R₃₂₈, R_{F1}, R_{F2}, and R_{F3} is a single bond with *i;
adjacent two selected from R₃₂₁ to R₃₂₈ not being the single bond are mutually bonded to form a substituted or unsubstituted benzene ring, or not mutually bonded;
** represents a bonding position to L₃₁ or L₃₂;
when L₃₁ is a single bond, ** of the group represented by the formula (1-e), which is Ar₃₁, represents a bonding position to the central nitrogen atom N*; and
when L₃₂ is a single bond, ** of the group represented by the formula (1-e), which is Ar₃₂, represents a bonding position to the central nitrogen atom N*, 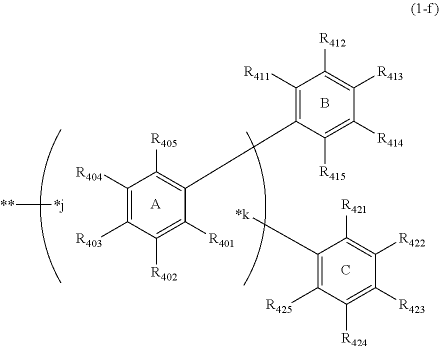
where, in the formula (1-f):
R₄₀₁ to R₄₀₅ are each independently a hydrogen atom, an unsubstituted alkyl group having 1 to 6 carbon atoms, or an unsubstituted phenyl group;
R₄₁₁ to R₄₁₅ and R₄₂₁ to R₄₂₅ are each independently a hydrogen atom or an unsubstituted alkyl group having 1 to 6 carbon atoms;
one selected from R₄₀₁ to R₄₀₅ is a single bond with *j;
another one selected from R₄₀₁ to R₄₀₅ is a single bond with *k;
adjacent two selected from R₄₀₁ to R₄₀₅ not being the single bond are not mutually bonded and thus form no cyclic structure;
adjacent two selected from R₄₁₁ to R₄₁₅ and R₄₂₁ to R₄₂₅ are mutually bonded to form a substituted or unsubstituted benzene ring, or not mutually bonded;
** represents a bonding position to L₃₁ or L₃₂;
when L₃₁ is a single bond, ** of the group represented by the formula (1-f), which is Ar₃₁, represents a bonding position to the central nitrogen atom N*; and
when L₃₂ is a single bond, ** of the group represented by the formula (1-f), which is Ar₃₂, represents a bonding position to the central nitrogen atom N*.

15: The organic electroluminescence device according to claim 1, wherein L₃₁ and L₃₂ are each independently a single bond or a substituted or unsubstituted phenylene group.

16: The organic electroluminescence device according to claim 1, wherein the triplet energy of the first host material T₁(H1) satisfies Numerical Formula 12 below:
$${T}_{1} ( H 1 ) > 2. eV . \qquad ( Numerical Formula 12 )$$

17: The organic electroluminescence device according to claim 1, wherein a triplet energy of the first luminescent compound T₁(D1) satisfies a Numerical Formula 14 below:
$$2.7 eV > {T}_{1} ( D 1 ) . \qquad ( Numerical Formula 14 )$$

18: The organic electroluminescence device according to claim 1, wherein a triplet energy of the second luminescent compound T₁(D2) satisfies Numerical Formula 14C below:
$$2.7 eV > {T}_{1} ( D 2 ) . \qquad ( Numerical Formula 14 C )$$

19: The organic electroluminescence device according to claim 1, wherein the triplet energy of the second host material T₁(H2) satisfies Numerical Formula 13B below:
$$1.9 eV > {T}_{1} ( H 2 ) . \qquad ( Numerical Formula 13 B )$$

20: The organic electroluminescence device according to claim 1, wherein the first host material comprises, in a molecule, at least one of a structure of Condition (i) below or a structure of Condition (ii) below:
Condition (i): a biphenyl structure including a first benzene ring and a second benzene ring linked to each other with a single bond, the first benzene ring and the second benzene ring in the biphenyl structure being further linked to each other by cross-linking at at least one site other than the single bond, and
Condition (ii): a first linking structure including a benzene ring and a naphthalene ring linked to each other with a single bond, the benzene ring and the naphthalene ring in the first linking structure being each independently further fused or not fused with a monocyclic ring or fused ring, the benzene ring and the naphthalene ring in the first linking structure being further linked to each other by cross-linking at at least one site other than the single bond.

21: The organic electroluminescence device according to claim 20, wherein the first host material comprises, in a molecule, the structure of Condition (i).

22: The organic electroluminescence device according to claim 21, wherein the first benzene ring and the second benzene ring in the biphenyl structure are further linked to each other by the cross-linking of Condition (i) at one site other than the single bond.

23: The organic electroluminescence device according to claim 21, wherein the cross-linking of Condition (i) comprises a double bond.

24: The organic electroluminescence device according to claim 21, wherein
the first benzene ring and the second benzene ring in the biphenyl structure are further linked to each other by the cross-linking of Condition (i) at two sites other than the single bond, and
the cross-linking of Condition (i) comprises no double bond.

25: The organic electroluminescence device according to claim 20, wherein the first host material comprises, in a molecule, the structure of Condition (ii).

26: The organic electroluminescence device according to claim 25, wherein the cross-linking of Condition (ii) comprises a double bond.

27: The organic electroluminescence device according to claim 1, wherein the first host material is a compound selected from the group consisting of a compound represented by a formula (H11), a compound represented by a formula (H12), a compound represented by a formula (H13), a compound represented by a formula (H14), a compound represented by a formula (H15), and a compound represented by a formula (H16) below, 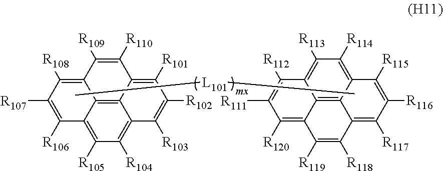
where, in the formula (H11):
R₁₀₁ to R₁₁₀ and R₁₁₁ to R₁₂₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
one of R₁₀₁ to R₁₁₀ represents a bonding position to L₁₀₁, and one of R₁₁₁ to R₁₂₀ represents a bonding position to L₁₀₁;
L₁₀₁ is a single bond, a substituted or unsubstituted arylene group having 6 to 24 ring carbon atoms, or a substituted or unsubstituted divalent heterocyclic group having 5 to 24 ring atoms;
mx is 0, 1, 2, 3, 4, or 5; and
when two or more L₁₀₁ are present, the two or more L₁₀₁ are mutually the same or different, 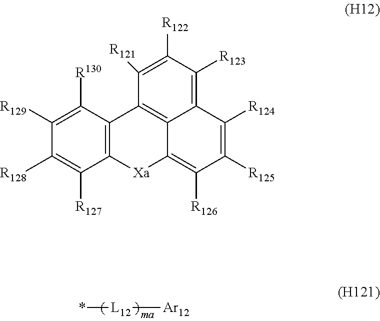
where, in the formula (H12):
Xa is an oxygen atom, a sulfur atom, C(R₁₂₀₁)(R₁₂₀₂), or Si(R₁₂₀₃)(R₁₂₀₄);
R₁₂₀₁ to R₁₂₀₄ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms;
at least one combination of adjacent two or more of R₁₂₁ to R₁₃₀ are mutually bonded to form a substituted or unsubstituted monocyclic ring, mutually bonded to form a substituted or unsubstituted fused ring, or not mutually bonded;
R₁₂₁ to R₁₃₀ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, a substituted or unsubstituted heterocyclic group having 5 to 50 ring atoms, or a group represented by a formula (H121) above;
at least one of R₁₂₁ to R₁₃₀ is a group represented by the formula (H121);
when a plurality of groups represented by the formula (H121) are present, the plurality of groups represented by the formula (H121) are mutually the same or different;
L₁₂ is a single bond, a substituted or unsubstituted arylene group having 6 to 50 ring carbon atoms, or a substituted or unsubstituted divalent heterocyclic group having 5 to 50 ring atoms;
ma is 0, 1, 2 or 3;
when two or more L₁₂ are present, the two or more L₁₂ are mutually the same or different;
Ar₁₂ is a substituted or unsubstituted aryl group having 6 to 50 ring carbon atoms, or a substituted or unsubstituted heterocyclic group having 5 to 50 ring atoms;
when two or more Ar₁₂ are present, the two or more Ar₁₂ are mutually the same or different; and
* in the formula (H121) represents a bonding position, 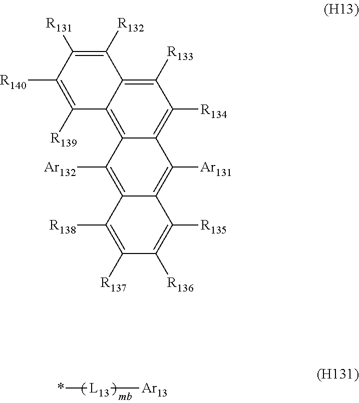
where, in the formula (H13):
at least one combination of adjacent two or more of R₁₃₁ to R₁₃₄ and R₁₃₉ to R₁₄₀ are mutually bonded to form a substituted or unsubstituted monocyclic ring, or not mutually bonded;
at least one combination of adjacent two or more of R₁₃₅ to R₁₃₈ are mutually bonded to form a substituted or unsubstituted monocyclic ring, or not mutually bonded;
Ar₁₃₁, Ar₁₃₂, and R₁₃₁ to R₁₄₀ not forming the substituted or unsubstituted monocyclic ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by a formula (H131) above;
at least one of R₁₃₁ to R₁₄₀, Ar₁₃₁, or Ar₁₃₂ is a group represented by the formula (H131);
when a plurality of groups represented by the formula (H131) are present, the plurality of groups represented by the formula (H131) are mutually the same or different;
L₁₃ is a single bond, a substituted or unsubstituted arylene group having 6 to 50 ring carbon atoms, or a substituted or unsubstituted divalent heterocyclic group having 5 to 50 ring atoms;
Ar₁₃ is a substituted or unsubstituted aryl group having 6 to 50 ring carbon atoms, or a substituted or unsubstituted heterocyclic group having 5 to 50 ring atoms;
mb is 0, 1, 2, 3, 4, or 5;
when two or more L₁₃ are present, the two or more L₁₃ are mutually the same or different;
when two or more Ar₁₃ are present, the two or more Ar₁₃ are mutually the same or different; and
* in the formula (H131) represents a bonding position to a benz[a]anthracene ring in the formula (H13), 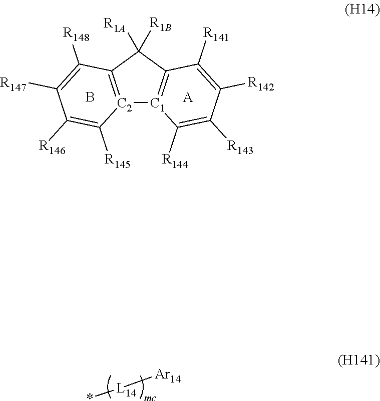
where, in the formula (H14):
R_{1A} and R_{1B} are each independently a substituted or unsubstituted alkyl group having 1 to 15 carbon atoms, a substituted or unsubstituted aryl group having 6 to 17 ring carbon atoms, or a substituted or unsubstituted heterocyclic group having 5 to 17 ring atoms;
at least one of R_{1A} or R_{1B} is a substituted or unsubstituted alkyl group having 1 to 15 carbon atoms;
a combination of adjacent two or more of R₁₄₁ to R₁₄₄ or a combination of adjacent two or more of R₁₄₅ to R₁₄₈ are mutually bonded to form a substituted or unsubstituted monocyclic ring, or mutually bonded to form a substituted or unsubstituted fused ring;
when a substituted or unsubstituted monocyclic ring or a substituted or unsubstituted fused ring is formed with a ring A, a group represented by a formula (H141) above is bonded to a carbon atom bonded to R₁₄₂ or, of carbon atoms forming the monocyclic ring with the ring A and the fused ring with the ring A, a carbon atom farthest from a carbon atom C₁ of the ring A, the carbon atom C₁ being bonded with a single bond to a carbon atom C₂ of a ring B;
when a substituted or unsubstituted monocyclic ring or a substituted or unsubstituted fused ring is formed with not the ring A but the ring B, a group represented by the formula (H141) is bonded to a carbon atom bonded to R₁₄₂; and
R₁₄₂ not being the group represented by the formula (H141), and R₁₄₁, R₁₄₃, R₁₄₄, and R₁₄₅ to R₁₄₈ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 17 ring carbon atoms, or a substituted or unsubstituted heterocyclic group having 5 to 17 ring atoms,
in the formula (H141):
Ar₁₄ is a substituted or unsubstituted aryl group having four or more fused rings or a substituted or unsubstituted heterocyclic group having four or more fused rings;
L₁₄ is a single bond, a substituted or unsubstituted arylene group having 6 to 17 ring carbon atoms, or a substituted or unsubstituted divalent heterocyclic group having 5 to 17 ring atoms;
mc is 0, 1, or 2;
* represents a bonding position to an atom forming a ring of the formula (H14); and
the compound represented by the formula (H14) does not have, in a molecule, three or more groups of a substituted or unsubstituted aryl group having four or more fused rings and a substituted or unsubstituted heterocyclic group having four or more fused rings, 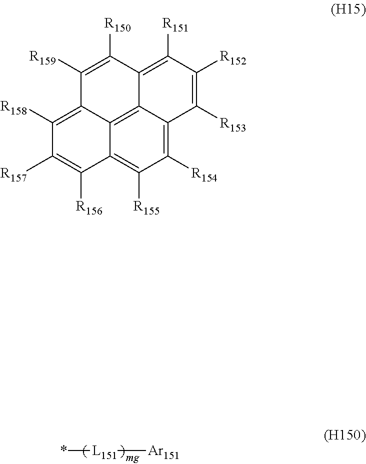
where, in the formula (H15):
R₁₅₀ to R₁₅₉ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by a formula (H150) above;
at least one of R₁₅₀ to R₁₅₉ is a group represented by the formula (H150);
when a plurality of groups represented by the formula (H150) are present, the plurality of groups represented by the formula (H150) are mutually the same or different;
L₁₅₁ is a single bond, a substituted or unsubstituted arylene group having 6 to 50 ring carbon atoms, or a substituted or unsubstituted divalent heterocyclic group having 5 to 50 ring atoms;
Ar₁₅₁ is a substituted or unsubstituted aryl group having 6 to 50 ring carbon atoms, or a substituted or unsubstituted heterocyclic group having 5 to 50 ring atoms;
mg is 0, 1, 2, 3, 4, or 5;
when two or more L₁₅₁ are present, the two or more L₁₅₁ are mutually the same or different;
when two or more Ar₁₅₁ are present, the two or more Ar₁₅₁ are mutually the same or different; and
* in the formula (H150) represents a bonding position to a pyrene ring in the formula (H15), 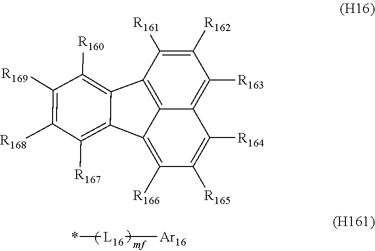
where, in the formula (H16):
at least one combination of adjacent two or more of R₁₆₀ to R₁₆₉ are mutually bonded to form a substituted or unsubstituted monocyclic ring, mutually bonded to form a substituted or unsubstituted fused ring, or not mutually bonded;
R₁₆₀ to R₁₆₉ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, a substituted or unsubstituted heterocyclic group having 5 to 50 ring atoms, or a group represented by a formula (H161) above;
at least one of a substituent, if present, for the substituted or unsubstituted monocyclic ring, a substituent, if present, for the substituted or unsubstituted fused ring, or R₁₆₀ to R₁₆₉ is a group represented by the formula (H161);
when a plurality of groups represented by the formula (H161) are present, the plurality of groups represented by the formula (H161) are mutually the same or different;
L₁₆ is a single bond, a substituted or unsubstituted arylene group having 6 to 50 ring carbon atoms, or a substituted or unsubstituted divalent heterocyclic group having 5 to 50 ring atoms;
Ar₁₆ is a substituted or unsubstituted aryl group having 6 to 50 ring carbon atoms, or a substituted or unsubstituted heterocyclic group having 5 to 50 ring atoms;
mf is 0, 1, 2, 3, 4, or 5;
when two or more L₁₆ are present, the two or more L₁₆ are mutually the same or different;
when two or more Ar₁₆ are present, the two or more Ar₁₆ are mutually the same or different; and
* in the formula (H161) represents a bonding position to a ring represented by the formula (H16);
in the first host material, R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

28: The organic electroluminescence device according to claim 27, wherein the first host material is a compound selected from the group consisting of a compound represented by a formula (H111), a compound represented by a formula (H122), a compound represented by a formula (H132), and a compound represented by a formula (H133) below, 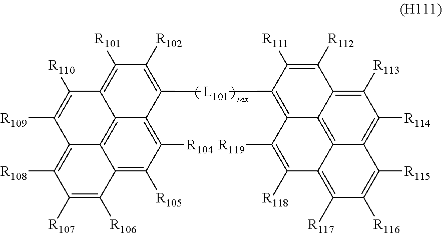
where, in the formula (H111):
R₁₀₁, R₁₀₂, R₁₀₄ to R₁₁₁, and R₁₁₁ to Riis are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
L₁₀₁ and mx respectively represent the same as L₁₀₁ and mx in the formula (H11), 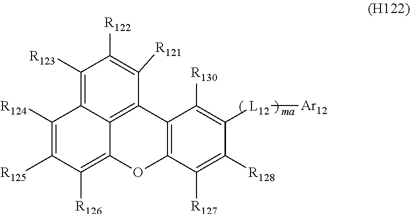
where, in the formula (H122):
R₁₂₁ to R₁₂₈ and R₁₃₀ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a nitro group, a substituted or unsubstituted aryl group having 6 to 50 ring carbon atoms, or a substituted or unsubstituted heterocyclic group having 5 to 50 ring atoms; and
Ar₁₂, L₁₂, and ma respectively represent the same as Ar₁₂, L₁₂, and ma in the formula (H121), 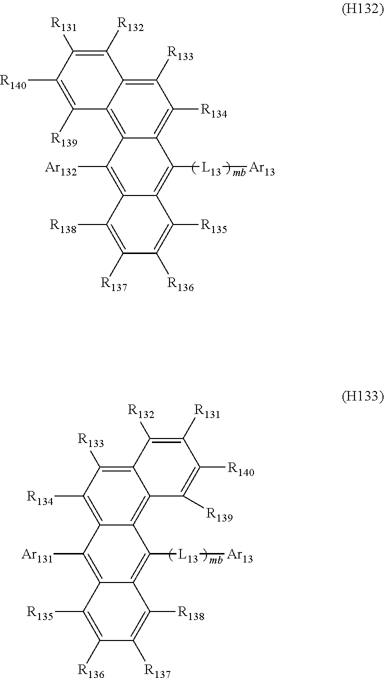
where, in the formulae (H132) and (H133):
R₁₃₁ to R₁₄₀, Ar₁₃₁, and Ar₁₃₂ are each a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms; and
L₁₃, Ar₁₃, and mb respectively represent the same as L₁₃, Ar₁₃, and mb in the formula (H131).

29: The organic electroluminescence device according to claim 1, wherein the first host material is a compound having, in a molecule, at least one group represented by a formula (HX1) below, 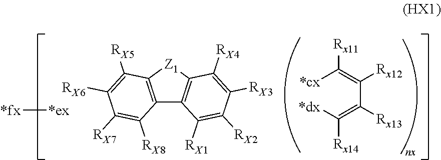
where, in the formula (HX1):
R_{X1} to R_{X8} and R_{X11} to R_{X14} are each independently a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms;
R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different;
when two or more R₉₀₇ are present, the two or more R₉₀₇ are mutually the same or different;
nx is 0 or 1;
when nx is 0, one selected from R_{X1} to R_{X8} is a single bond with *ex;
when nx is 1, one of R_{X1} and R_{X2}, one of R_{X2} and R_{X3}, or one of R_{X3} and R_{X4} is a single bond with *cx; the other of R_{X1} and R_{X2}, the other of R_{X2} and R_{X3}, or the other of R_{X3} and R_{X4} is a single bond with *dx; and one selected from R_{X5} to R_{X8}, R_{X11} to R_{X14}, and R_{X1} to R_{X4} being neither the single bond with *cx nor the single bond with *dx is a single bond with *ex;
Z₁ is an oxygen atom or a sulfur atom; and
*fx represents a bonding position to an atom in the first host material.

30-46. (canceled)

47: An electronic device comprising the organic electroluminescence device according to claim 1.
